(19) United States
(12) Patent Application Publication
(10) Pub. No.: US 2025/0263395 A1
(43) Pub. Date: Aug. 21, 2025

(54) COMPOUNDS THAT MEDIATE PROTEIN DEGRADATION AND METHODS OF USE THEREOF

(71) Applicant: Monte Rosa Therapeutics AG, Basel (CH)

(72) Inventors: Andreas Ritzén, Basel (CH); Thomas Ryckmans, Basel (CH); Laura Ann McAllister, Basel (CH); Vladimiras Oleinikovas, Basel (CH); Beatrice Ranieri, Basel (CH); Bernhard Fasching, Basel (CH); Elisa Liardo, Basel (CH)

(21) Appl. No.: 19/190,194

(22) Filed: Apr. 25, 2025

Related U.S. Application Data
(63) Continuation of application No. PCT/US23/77781, filed on Oct. 25, 2023.
(60) Provisional application No. 63/419,575, filed on Oct. 26, 2022.

(51) Int. Cl. C07D 401/14; C07D 401/10; C07D 413/14; C07F 7/18

(52) U.S. Cl. CPC C07D 401/14; C07D 401/10; C07D 413/14; C07F 7/1804

(57) ABSTRACT

Described herein, in part, are compounds that mediate the degradation of cyclin-dependent kinase 2 (CDK2), and are therefore useful in the treatment of various disorders, such as cancer.

### CROSS-REFERENCE TO RELATED APPLICATIONS

The application is a continuation of International Patent Application No. PCT/US2023/077781, filed Oct. 25, 2023, which claims priority to, U.S. Ser. No. 63/419,575, filed Oct. 26, 2022, the contents of which is incorporated herein by reference.

### BACKGROUND

The ubiquitin proteasome system can be manipulated with different small molecules to trigger targeted degradation of specific proteins of interest. Promoting the targeted degradation of pathogenic proteins using small molecule degraders is emerging as a new modality in the treatment of diseases. One such modality relies on redirecting the activity of E3 ligases such as cereblon (a phenomenon known as E3 reprogramming) using low molecular weight compounds, which have been termed molecular glues to promote the poly-ubiquitination and ultimately proteasomal degradation of new protein substrates involved in the development of diseases. The molecular glues bind to both the E3 ligase and the target protein, thereby mediating an alteration of the ligase surface and enabling an interaction with the target protein.

There exists a need for therapeutics that effectively mediate the degradation of certain proteins for the treatment of diseases.

### SUMMARY

Described herein, in part, are compounds contemplated as modulators of cereblon to mediate the degradation of a protein, and are therefore are useful in the treatment of disorders, such as cancer. In some embodiments, compounds of the present disclosure mediate the targeted degradation of the protein cyclin-dependent kinase 2 (CDK2).

In an aspect, provided herein is a compound of Formula (I):

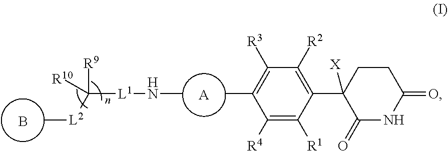

or a pharmaceutically acceptable salt thereof, wherein: X is selected from H and deuterium; L¹ is selected from the group consisting of:

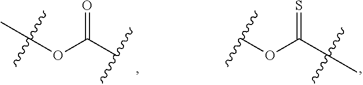 [SMILES: *OC(*)=O ; *OC(*)=S]

and 5-6 membered heteroaryl; L² is selected from a bond and

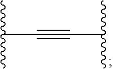 [SMILES: *C#C*]

each of R¹, R², R³, and R⁴ is independently selected from the group consisting of hydrogen, halogen, C₁₋₆alkoxy, cyano, hydroxy, C₃₋₆ cycloalkyl, and C₁₋₆alkyl; ring A is selected from C₃₋₆ cycloalkyl and 3 to 6 membered heterocyclyl, wherein each of C₃₋₆cycloalkyl and 3 to 6 membered heterocyclyl is optionally substituted with one or more occurrences of R⁵; each occurrence of R⁵ is independently selected from the group consisting of hydrogen, C₁₋₆ alkyl, hydroxy, and oxo, wherein C₁₋₆ alkyl is optionally substituted with one or more occurrences of halogen; ring B is selected from the group consisting of C₃₋₁₂ cycloalkyl, 3 to 10 membered heterocyclyl, aryl, and heteroaryl, wherein each of C₃₋₁₂ cycloalkyl, 3 to 10 membered heterocyclyl, aryl, and heteroaryl is optionally substituted with one or more occurrences of R⁶; each occurrence of R⁶ is independently selected from the group consisting of halogen cyano, C₁₋₆alkoxy, C₁₋₆alkyl, —C(O)R⁷, —C(O)NR⁷R⁸, —S(O)₂R⁷, pyridine,

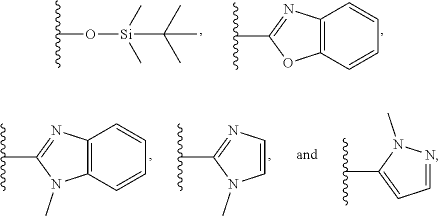

wherein each C₁₋₆alkyl, C₁₋₆alkoxy, and pyridine is optionally substituted with one or more occurrences of a substitutent selected from C₁₋₆alkyl and halogen; each occurrence of R⁷ is independently selected from the group consisting of C₁₋₆alkyl, phenyl, cyclopropane, an N-linked C₃₋₉ heterocycloalkyl, an N-linked heteroaryl,

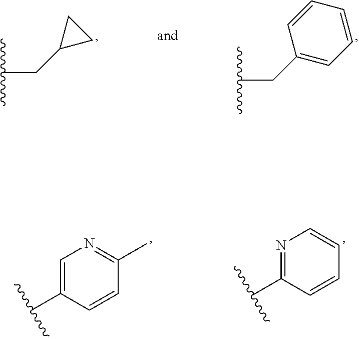

wherein R⁷ is optionally substituted with one or more occurrences of a substituent selected from the group consisting of of C₁₋₆alkyl, halogen, cyano, trifluoro(methoxy)methane, and C₁₋₆alkoxy (e.g., methoxy); each occurrence of R⁸, R⁹, and R¹⁰ is independently selected from hydrogen, deuterium, C₁₋₆alkyl, and deuterated C₁₋₆alkyl (e.g., —CD₃); and n is an integer selected from the group consisting of 0, 1, 2, and 3.

In an aspect, described herein is a pharmaceutical composition comprising a compound described herein, or pharmaceutically acceptable salt thereof, and a pharmaceutically acceptable excipient.

In an aspect, described herein is a method of degrading CDK2 in a subject suffering from cancer, comprising administering to the subject an effective amount of a compound described herein, or pharmaceutically acceptable salt thereof, or a pharmaceutical composition described herein.

In an aspect, described herein is a method of treating cancer in a subject in need thereof, comprising administering to the subject a therapeutically effective amount of a compound described herein, or pharmaceutically acceptable salt thereof, or a pharmaceutical composition described herein.

In an aspect, described herein is a method of treating a solid tumor in a subject in need thereof, comprising administering to the subject a therapeutically effective amount of a compound described herein, or pharmaceutically acceptable salt thereof, or a pharmaceutical composition described herein.

In an aspect, described herein is a method of treating a liquid tumor in a subject in need thereof, comprising administering to the subject a therapeutically effective amount of a compound described herein, or pharmaceutically acceptable salt thereof, or a pharmaceutical composition described herein.

### DETAILED DESCRIPTION

The features and other details of the disclosure will now be more particularly described. Certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

### Compounds

In one aspect, described herein is a compound of Formula (I):

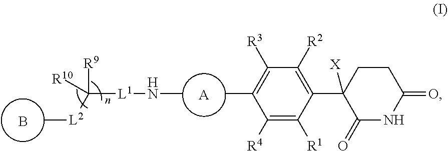

or a pharmaceutically acceptable salt thereof, wherein: X is selected from H and deuterium; L¹ is selected from the group consisting of:

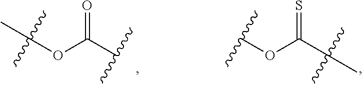 [SMILES: *OC(*)=O ; *OC(*)=S]

and 5-6 membered heteroaryl; L² is selected from a bond and

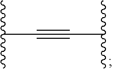 [SMILES: *C#C*]

each of R¹, R², R³, and R⁴ is independently selected from the group consisting of hydrogen, halogen, C₁₋₆alkoxy, cyano, hydroxy, C₃₋₆ cycloalkyl, and C₁₋₆alkyl; ring A is selected from C₃₋₆ cycloalkyl and 3 to 6 membered heterocyclyl, wherein each of C₃₋₆cycloalkyl and 3 to 6 membered heterocyclyl is optionally substituted with one or more occurrences of R⁵; each occurrence of R⁵ is independently selected from the group consisting of hydrogen, C₁₋₆ alkyl, hydroxy, and oxo, wherein C₁₋₆ alkyl is optionally substituted with one or more occurrences of halogen; ring B is selected from the group consisting of C₃₋₁₂ cycloalkyl, 3 to 10 membered heterocyclyl, aryl, and heteroaryl, wherein each of C₃₋₁₂ cycloalkyl, 3 to 10 membered heterocyclyl, aryl, and heteroaryl is optionally substituted with one or more occurrences of R⁶; each occurrence of R⁶ is independently selected from the group consisting of halogen, cyano, C₁₋₆alkoxy, C₁₋₆alkyl, —C(O)R⁷, —C(O)NR⁷R⁸, —S(O)₂R⁷, pyridine,

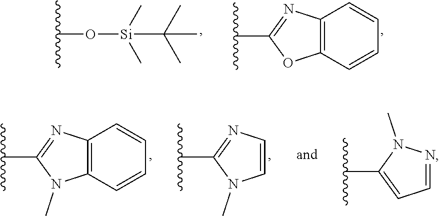

wherein each C₁₋₆alkyl, C₁₋₆alkoxy, and pyridine is optionally substituted with one or more occurrences of a substitutent selected from C₁₋₆alkyl and halogen; each occurrence of R⁷ is independently selected from the group consisting of C₁₋₆alkyl, phenyl, cyclopropane, an N-linked C₃₋₉ heterocycloalkyl, an N-linked heteroaryl,

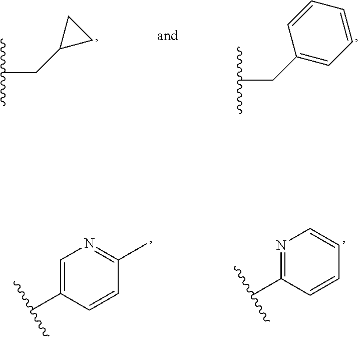

wherein R⁷ is optionally substituted with one or more occurrences of a substituent selected from the group consisting of of C₁₋₆alkyl, halogen, cyano, trifluoro(methoxy)methane, and C₁₋₆alkoxy (e.g., methoxy); each occurrence of R⁸, R⁹, and R¹⁰ is independently selected from hydrogen, deuterium, C₁₋₆alkyl, and deuterated C₁₋₆alkyl (e.g., —CD₃); and n is an integer selected from the group consisting of 0, 1, 2, and 3.

In some embodiments, ring A is selected from the group consisting of:

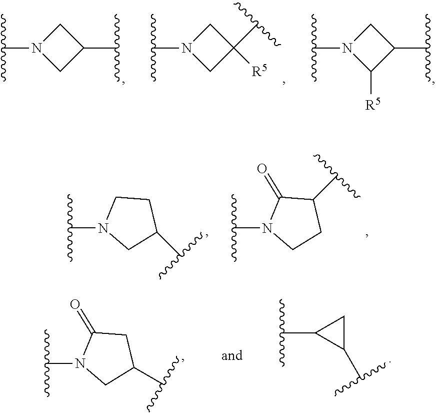

In some embodiments, ring B is selected from the group consisting of C₃₋₁₂ cycloalkyl, 3 to 10 membered heterocyclyl, and aryl, wherein each of C₃₋₁₂ cycloalkyl, 3 to 10 membered heterocyclyl, and aryl is substituted with one or more occurrences of R⁶.

In some embodiments, ring B is selected from the group consisting of:

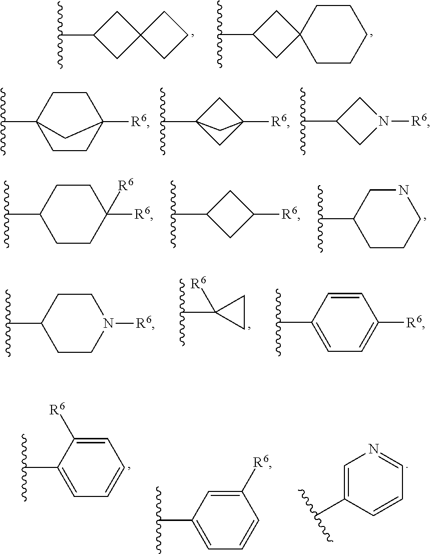

In some embodiments, ring B is

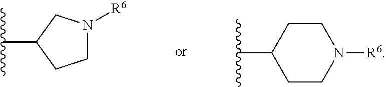

In some embodiments, ring B is

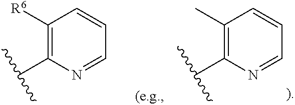

In some embodiments, ring B is

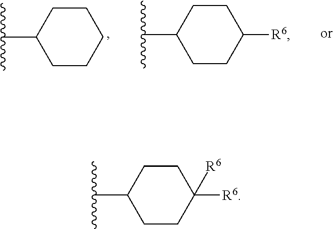

In some embodiments, R⁶ is selected from the group consisting of Cl, F, —CN, —CH₃, —CF₃, —CH(CH₃)₂, —OCH₃, —OC(CH₃)₃, —OCF₃, and —O—Si(CH₃)₂C(CH₃)₃.

In some embodiments, R⁶ is —C(O)R⁷, wherein R⁷ is selected from the group consisting of phenyl,

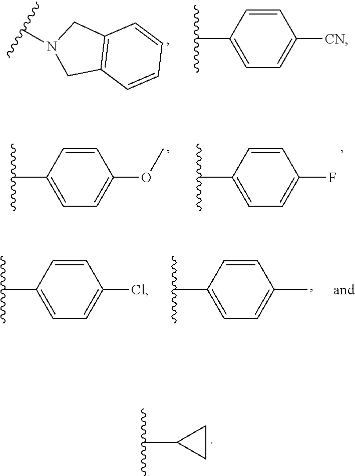

In some embodiments, R⁶ is —C(O)NR⁷R⁸, wherein R⁷ is selected from the group consisting of methyl, phenyl,

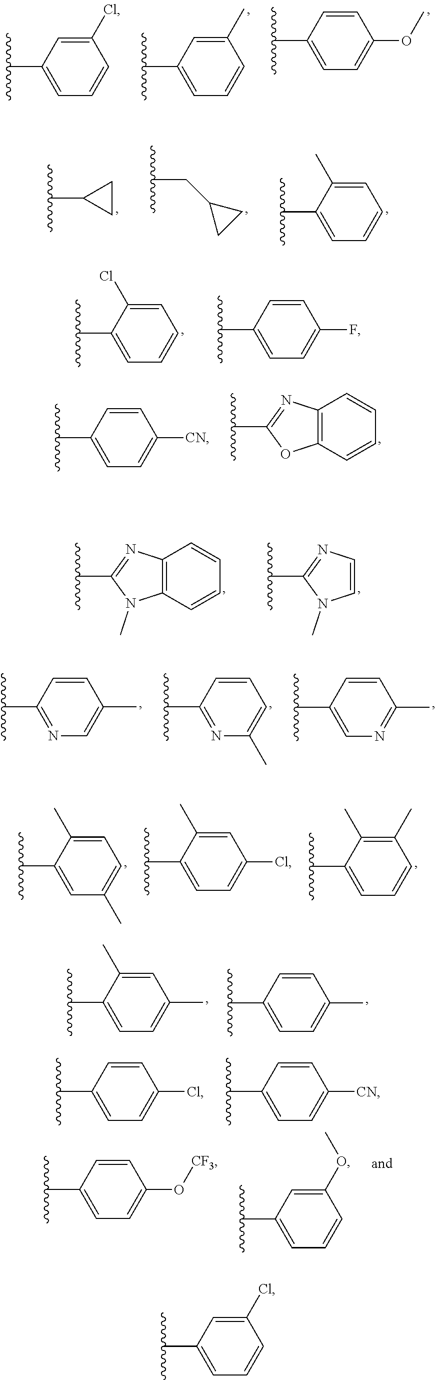

and R⁸ is CH₃ or CD₃.

In some embodiments, R⁶ is —S(O)₂R⁷, wherein R⁷ is

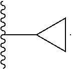 [SMILES: *C1CC1]

In some embodiments, L¹ is selected from the group consisting of:

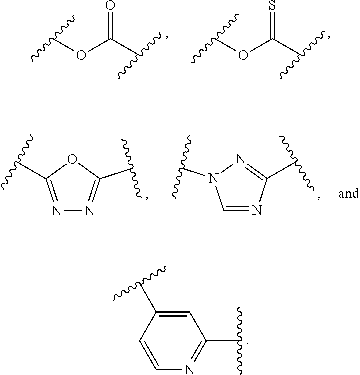 [SMILES: *OC(*)=O ; *OC(*)=S ; *c1nnc(*)o1 ; *c1ncn(*)n1 ; *c1ccnc(*)c1]

In some embodiments, L¹ is

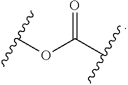 [SMILES: *OC(*)=O]

In some embodiments, the compound is a compound of Formula (I-A):

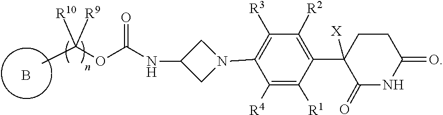

In some embodiments, the compound is a compound of Formula (I-B):

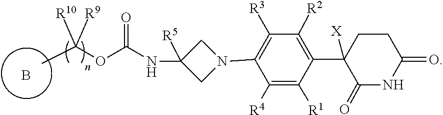

In some embodiments, the compound is a compound of Formula (I-C):

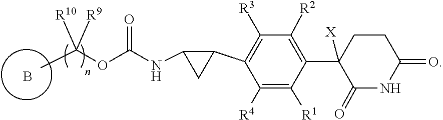

In some embodiments, the compound is a compound of Formula (I-D):

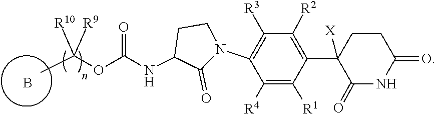

In some embodiments, the compound is a compound of Formula (I-E):

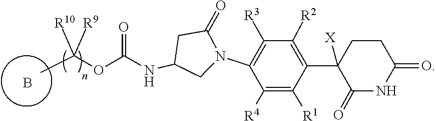

In some embodiments, the compound is a compound of Formula (I-F):

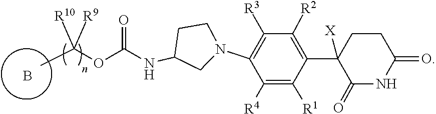

In some embodiments, the compound is a compound of Formula (I-G):

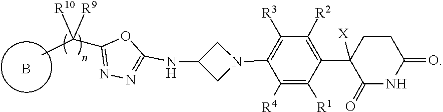

In some embodiments, the compound is a compound of Formula (I-H):

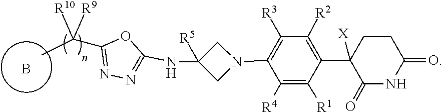

In some embodiments, the compound is a compound of Formula (I-I):

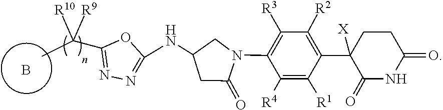

In some embodiments, the compound is a compound of Formula (I-I-0):

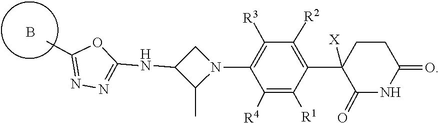

In some embodiments, the compound is a compound of Formula (I-I-1-1):

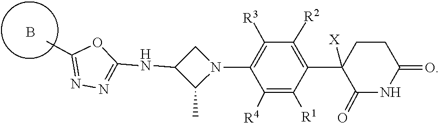

In some embodiments, the compound is a compound of Formula (I-I-1-2):

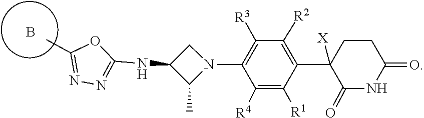

In some embodiments, the compound is a compound of Formula (I-I-2):

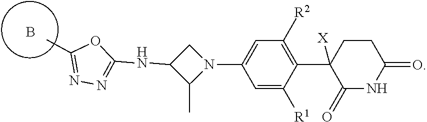

In some embodiments, the compound is a compound of Formula (I-I-2-1):

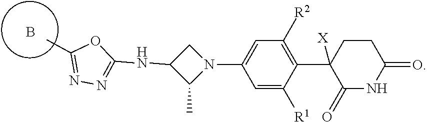

In some embodiments, the compound is a compound of Formula (I-I-2-2):

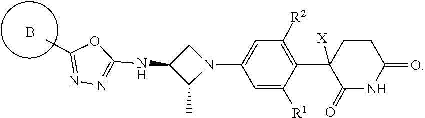

In some embodiments, the compound is a compound of Formula (I-I-3):

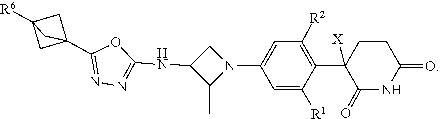

In some embodiments, the compound is a compound of Formula (I-I-3-1):

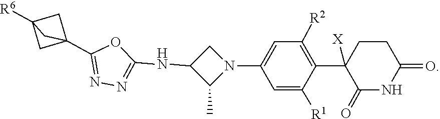

In some embodiments, the compound is a compound of Formula (I-I-3-2):

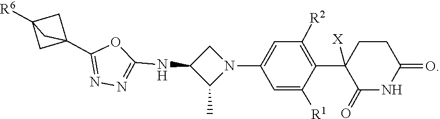

In some embodiments, the compound is a compound of Formula (I-I-4):

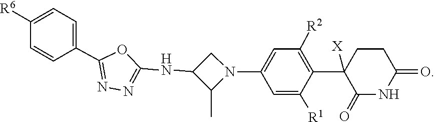

In some embodiments, the compound is a compound of Formula (I-I-4-1):

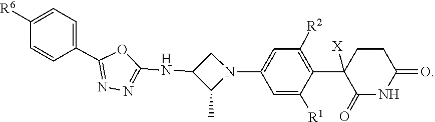

In some embodiments, the compound is a compound of Formula (I-I-4-2):

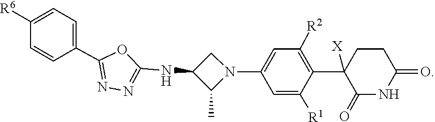

In some embodiments, the compound is a compound of Formula (I-I-5):

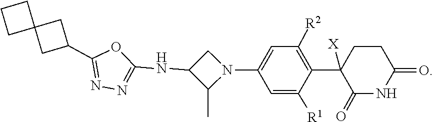

In some embodiments, the compound is a compound of Formula (I-I-5-1):

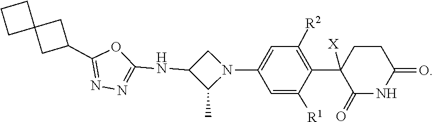

In some embodiments, the compound is a compound of Formula (I-I-5-2):

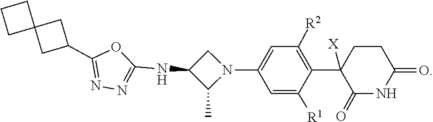

In some embodiments, the compound is a compound of Formula (I-J):

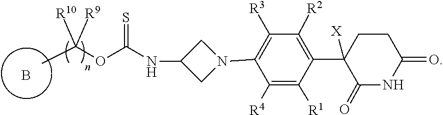

In some embodiments, the compound is a compound of Formula (I-K):

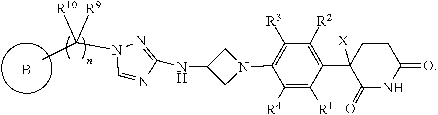

In some embodiments, the compound is a compound of Formula (I-L):

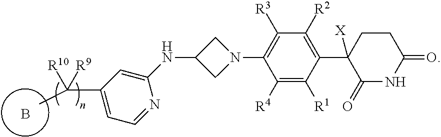

In some embodiments, the compound is a compound of Formula (I-i):

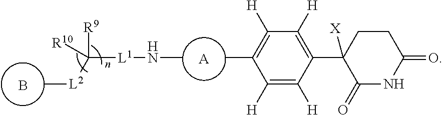

In some embodiments, the compound is a compound of Formula (I-ii):

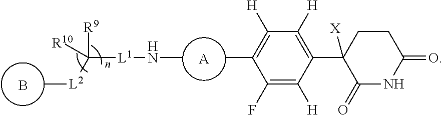

In some embodiments, the compound is a compound of Formula (I-iii):

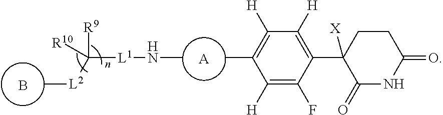

In some embodiments, the compound is a compound of Formula (I-iv):

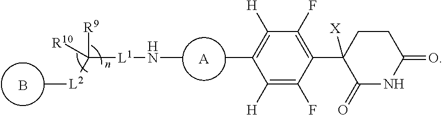

In some embodiments, the compound is a compound of Formula (I-v):

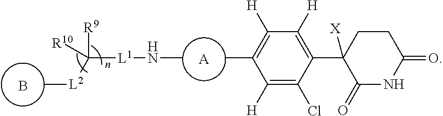

In some embodiments, the compound is a compound of Formula (I-vi):

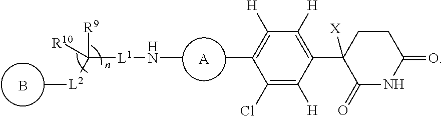

In some embodiments, the compound is a compound of Formula (I-vii):

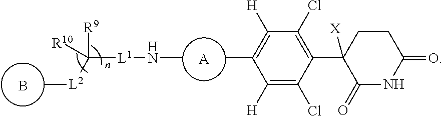

In some embodiments, the compound is a compound of Formula (I-viii):

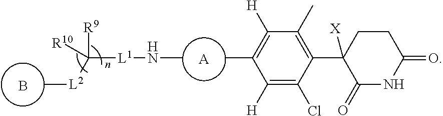

In some embodiments, the compound is a compound of Formula (I-ix):

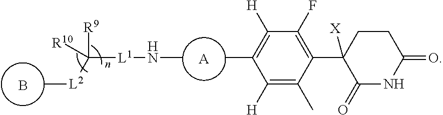

In some embodiments, the compound is a compound of Formula (I-x):

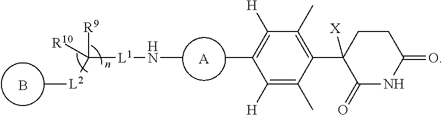

In some embodiments, the compound is a compound of Formula (I-xi):

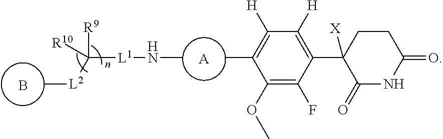

In some embodiments, the compound is a compound of Formula (I-xii):

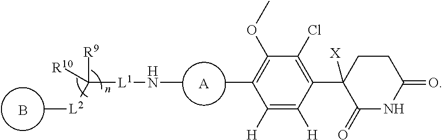

In some embodiments, the compound is a compound of Formula (I-xiii):

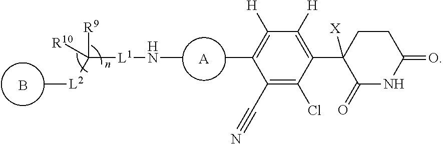

In some embodiments, the compound is a compound of Formula (I-xix):

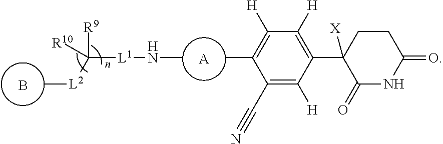

In some embodiments, the compound is a compound of Formula (I-xx):

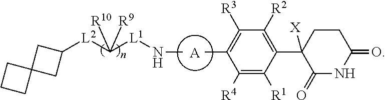

In some embodiments, the compound is a compound of Formula (I-xxi):

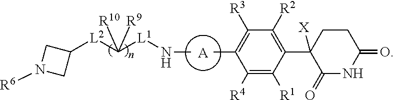

In some embodiments, the compound is a compound of Formula (I-xxii):

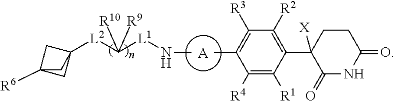

In some embodiments, the compound is a compound of Formula (I-xxiii):

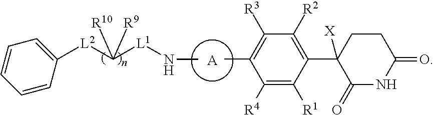

In some embodiments, the compound is a compound of Formula (I-xxiv):

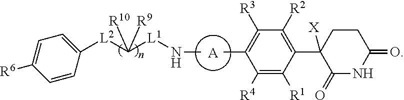

In some embodiments, the compound is a compound of Formula (I-xxv):

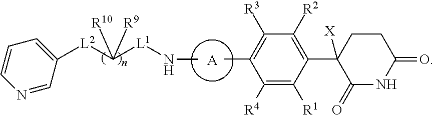

In some embodiments, X is H.

In some embodiments, L² is a bond.

In some embodiments, L² is a

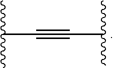 [SMILES: *C#C*]

In some embodiments, R¹, R², R³, and R⁴ are H.

In some embodiments, R¹ is fluoro, R² is fluoro, R³ is H, and R⁴ is H.

In some embodiments, R⁹ and R¹⁰ are H.

In some embodiments, n is 3.

In some embodiments, n is 2.

In some embodiments, n is 1.

In some embodiments, n is 0.

In some embodiments, R⁷ is selected from the group consisting of: methyl, benzene, cyclopropane,

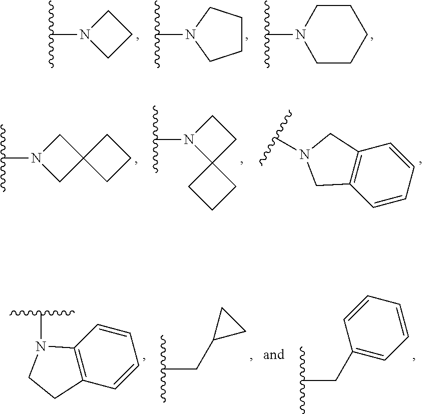

wherein each of

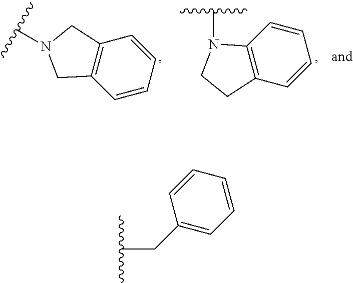 [SMILES: *N1Cc2ccccc2C1 ; *N1CCc2ccccc21 ; *Cc1ccccc1]

is optionally substituted with one or two occurrences selected from the group consisting of methyl, flourine, chlorine, cyano, and methoxy

In some embodiments, R⁷ is selected from the group consisting of methyl, phenyl,

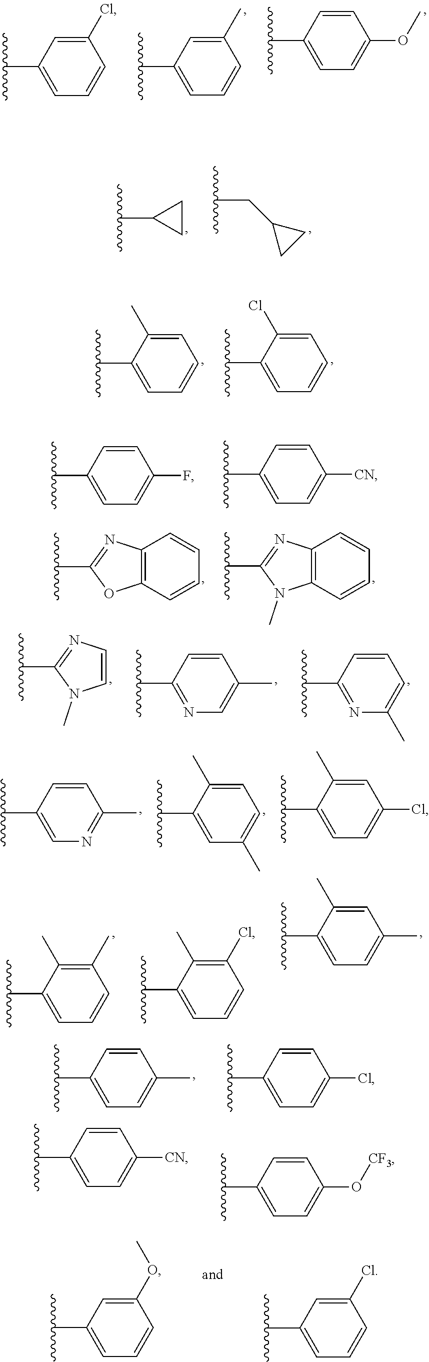

In some embodiments, R⁷ is phenyl optionally substituted by C₁₋₆alkyl (e.g., methyl), halogen, cyano, trifluoro(methoxy)methane, and C₁₋₆alkoxy (e.g., methoxy)

In some embodiments, each occurrence of R⁸, R⁹, and R¹⁰ is independently hydrogen or methyl. In some embodiments, R⁸ is methyl. In some embodiments, R⁸ is —CD₃.

In some embodiments, each occurrence of R⁸, R⁹, and R¹⁰ is independently deuterated C₁₋₆alkyl. In some embodiments, each occurrence of R⁸, R⁹, and R¹⁰ is independently —CD₃.

In some embodiments, the compound is a compound described in Table 1 below. Table 1 also includes the compound number of each compound in accordance with the contents of the present specification.

TABLE 1
Exemplary Compounds

| CmpdNo. | Structure |
|---|---|
| 1 | 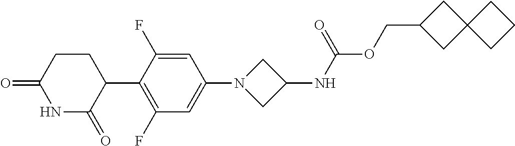 [SMILES: O=C1CCC(c2c(F)cc(N3CC(NC(=O)OCC4CC5(CCC5)C4)C3)cc2F)C(=O)N1] |
| 2 | 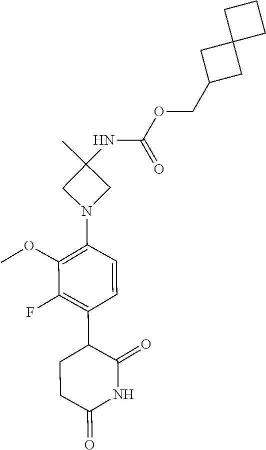 [SMILES: COc1c(N2CC(C)(NC(=O)OCC3CC4(CCC4)C3)C2)ccc(C2CCC(=O)NC2=O)c1F] |
| 3 | 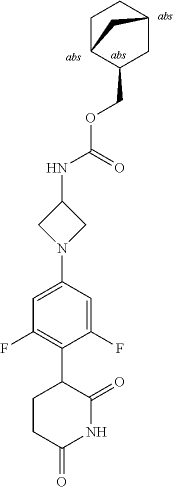 [SMILES: O=C1CCC(c2c(F)cc(N3CC(NC(=O)OC[C@@H]4C[C@H]5CC[C@@H]4C5)C3)cc2F)C(=O)N1] |
| 4 | 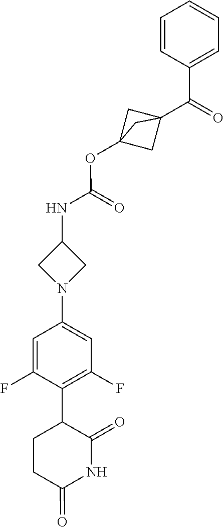 [SMILES: O=C1CCC(c2c(F)cc(N3CC(NC(=O)OC45CC(C(=O)c6ccccc6)(C4)C5)C3)cc2F)C(=O)N1] |
| 5 | 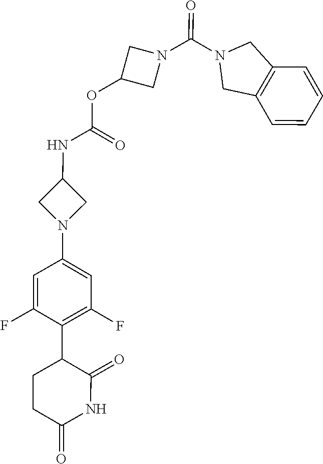 [SMILES: O=C1CCC(c2c(F)cc(N3CC(NC(=O)OC4CN(C(=O)N5Cc6ccccc6C5)C4)C3)cc2F)C(=O)N1] |
| 6 | 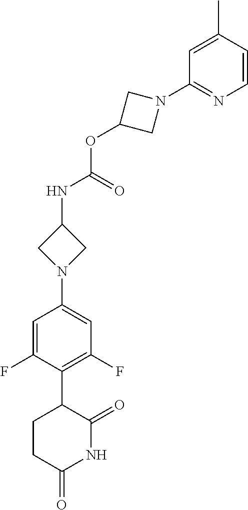 [SMILES: Cc1ccnc(N2CC(OC(=O)NC3CN(c4cc(F)c(C5CCC(=O)NC5=O)c(F)c4)C3)C2)c1] |
| 7 | 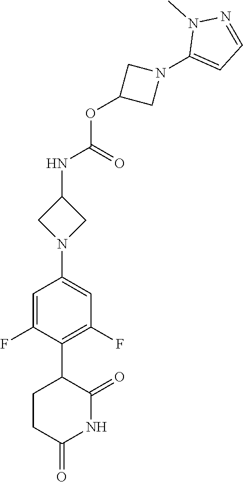 [SMILES: Cn1nccc1N1CC(OC(=O)NC2CN(c3cc(F)c(C4CCC(=O)NC4=O)c(F)c3)C2)C1] |
| 8 | 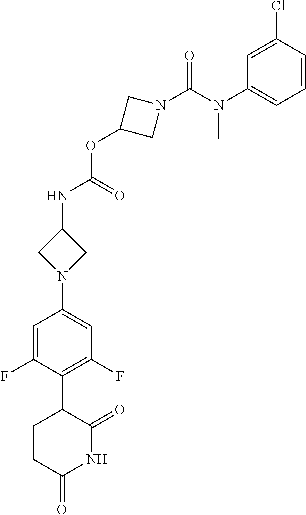 [SMILES: CN(C(=O)N1CC(OC(=O)NC2CN(c3cc(F)c(C4CCC(=O)NC4=O)c(F)c3)C2)C1)c1cccc(Cl)c1] |
| 9 | 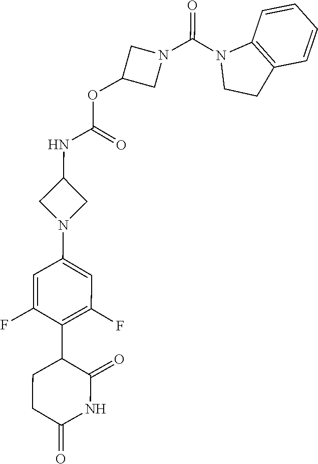 [SMILES: O=C1CCC(c2c(F)cc(N3CC(NC(=O)OC4CN(C(=O)N5CCc6ccccc65)C4)C3)cc2F)C(=O)N1] |
| 10 | 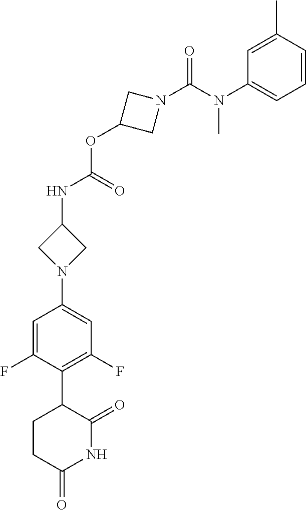 [SMILES: Cc1cccc(N(C)C(=O)N2CC(OC(=O)NC3CN(c4cc(F)c(C5CCC(=O)NC5=O)c(F)c4)C3)C2)c1] |
| 11 | 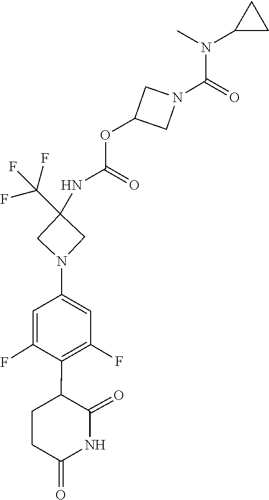 [SMILES: CN(C(=O)N1CC(OC(=O)NC2(C(F)(F)F)CN(c3cc(F)c(C4CCC(=O)NC4=O)c(F)c3)C2)C1)C1CC1] |
| 12 | 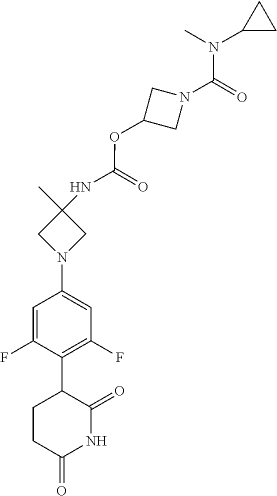 [SMILES: CN(C(=O)N1CC(OC(=O)NC2(C)CN(c3cc(F)c(C4CCC(=O)NC4=O)c(F)c3)C2)C1)C1CC1] |
| 13 | 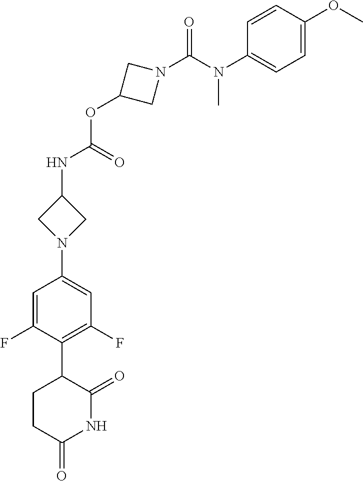 [SMILES: COc1ccc(N(C)C(=O)N2CC(OC(=O)NC3CN(c4cc(F)c(C5CCC(=O)NC5=O)c(F)c4)C3)C2)cc1] |
| 14 | 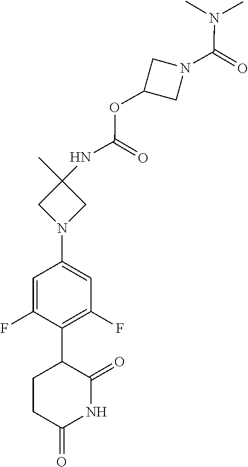 [SMILES: CN(C)C(=O)N1CC(OC(=O)NC2(C)CN(c3cc(F)c(C4CCC(=O)NC4=O)c(F)c3)C2)C1] |
| 15 | 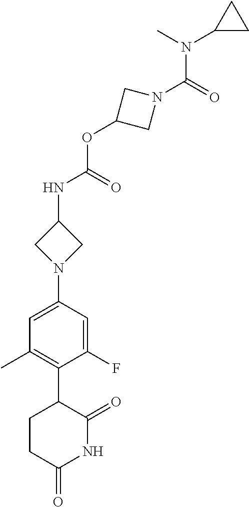 [SMILES: Cc1cc(N2CC(NC(=O)OC3CN(C(=O)N(C)C4CC4)C3)C2)cc(F)c1C1CCC(=O)NC1=O] |
| 16 | 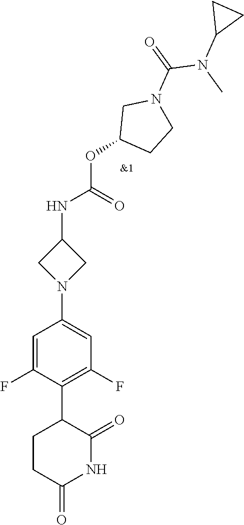 [SMILES: CN(C(=O)N1CC[C@H](OC(=O)NC2CN(c3cc(F)c(C4CCC(=O)NC4=O)c(F)c3)C2)C1)C1CC1] |
| 17 | 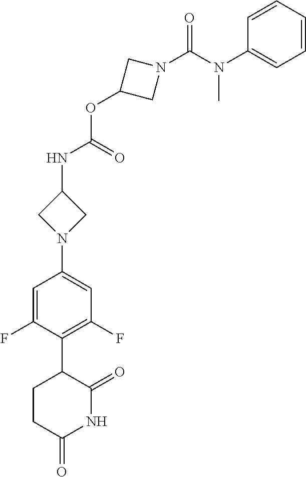 [SMILES: CN(C(=O)N1CC(OC(=O)NC2CN(c3cc(F)c(C4CCC(=O)NC4=O)c(F)c3)C2)C1)c1ccccc1] |
| 18 | 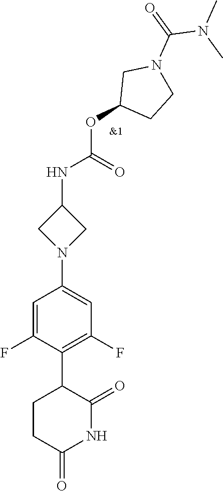 [SMILES: CN(C)C(=O)N1CC[C@@H](OC(=O)NC2CN(c3cc(F)c(C4CCC(=O)NC4=O)c(F)c3)C2)C1] |
| 19 | 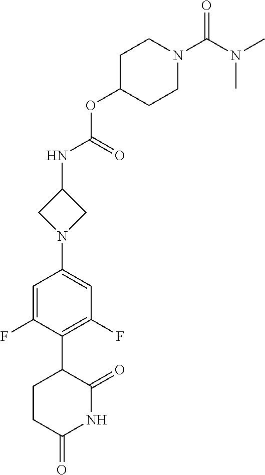 [SMILES: CN(C)C(=O)N1CCC(OC(=O)NC2CN(c3cc(F)c(C4CCC(=O)NC4=O)c(F)c3)C2)CC1] |
| 20 | 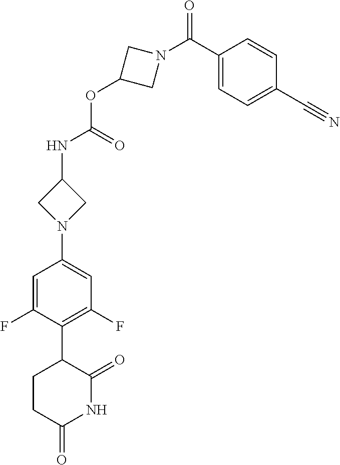 [SMILES: N#Cc1ccc(C(=O)N2CC(OC(=O)NC3CN(c4cc(F)c(C5CCC(=O)NC5=O)c(F)c4)C3)C2)cc1] |
| 21 | 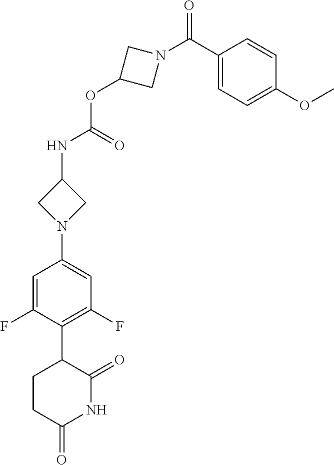 [SMILES: COc1ccc(C(=O)N2CC(OC(=O)NC3CN(c4cc(F)c(C5CCC(=O)NC5=O)c(F)c4)C3)C2)cc1] |
| 22 | 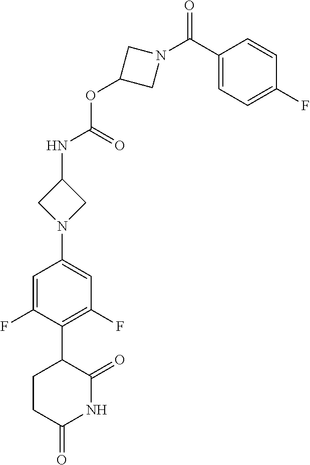 [SMILES: O=C1CCC(c2c(F)cc(N3CC(NC(=O)OC4CN(C(=O)c5ccc(F)cc5)C4)C3)cc2F)C(=O)N1] |
| 23 | 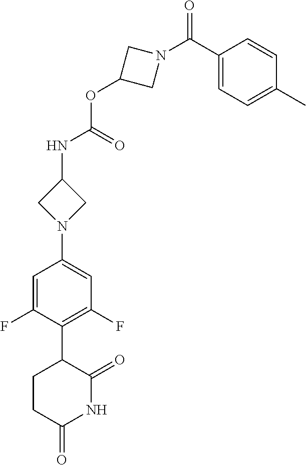 [SMILES: Cc1ccc(C(=O)N2CC(OC(=O)NC3CN(c4cc(F)c(C5CCC(=O)NC5=O)c(F)c4)C3)C2)cc1] |
| 24 | 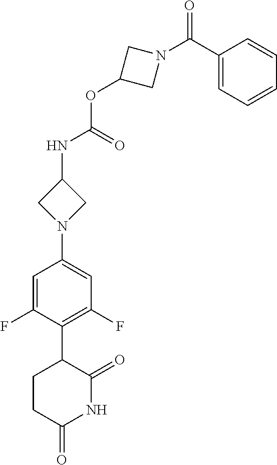 [SMILES: O=C1CCC(c2c(F)cc(N3CC(NC(=O)OC4CN(C(=O)c5ccccc5)C4)C3)cc2F)C(=O)N1] |
| 25 | 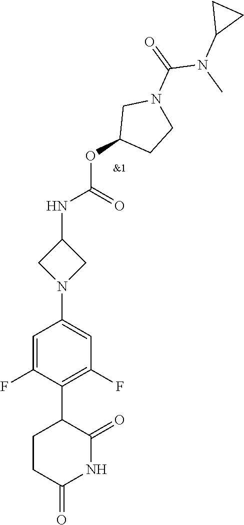 [SMILES: CN(C(=O)N1CC[C@@H](OC(=O)NC2CN(c3cc(F)c(C4CCC(=O)NC4=O)c(F)c3)C2)C1)C1CC1] |
| 26 | 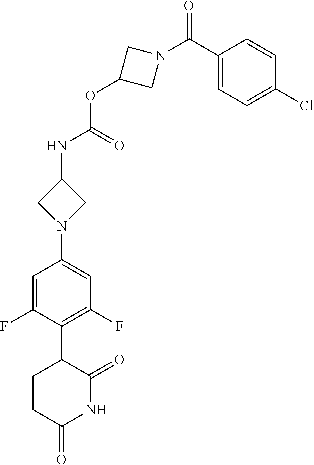 [SMILES: O=C1CCC(c2c(F)cc(N3CC(NC(=O)OC4CN(C(=O)c5ccc(Cl)cc5)C4)C3)cc2F)C(=O)N1] |
| 27 | 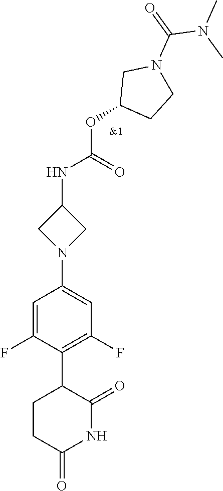 [SMILES: CN(C)C(=O)N1CC[C@H](OC(=O)NC2CN(c3cc(F)c(C4CCC(=O)NC4=O)c(F)c3)C2)C1] |
| 28 | 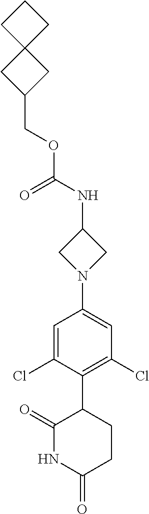 [SMILES: O=C1CCC(c2c(Cl)cc(N3CC(NC(=O)OCC4CC5(CCC5)C4)C3)cc2Cl)C(=O)N1] |
| 29 | 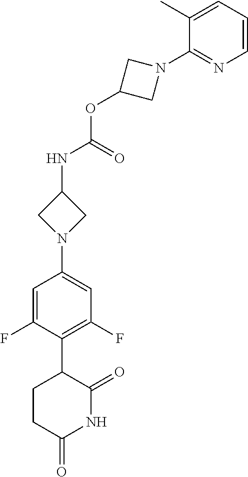 [SMILES: Cc1cccnc1N1CC(OC(=O)NC2CN(c3cc(F)c(C4CCC(=O)NC4=O)c(F)c3)C2)C1] |
| 30 | 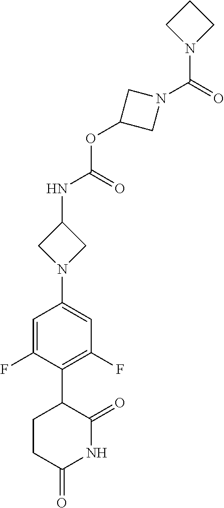 [SMILES: O=C1CCC(c2c(F)cc(N3CC(NC(=O)OC4CN(C(=O)N5CCC5)C4)C3)cc2F)C(=O)N1] |
| 31 | 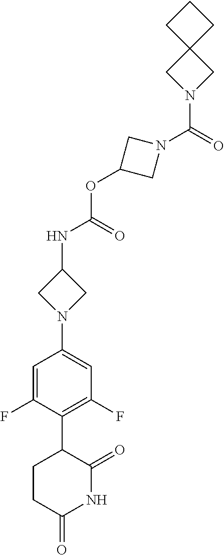 [SMILES: O=C1CCC(c2c(F)cc(N3CC(NC(=O)OC4CN(C(=O)N5CC6(CCC6)C5)C4)C3)cc2F)C(=O)N1] |
| 32 | 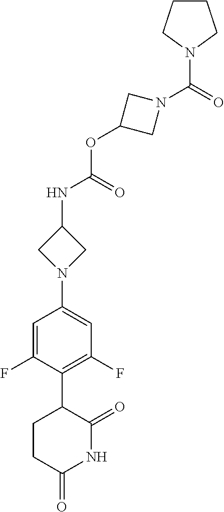 [SMILES: O=C1CCC(c2c(F)cc(N3CC(NC(=O)OC4CN(C(=O)N5CCCC5)C4)C3)cc2F)C(=O)N1] |
| 33 | 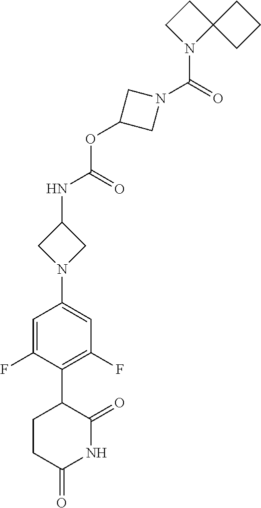 [SMILES: O=C1CCC(c2c(F)cc(N3CC(NC(=O)OC4CN(C(=O)N5CCC56CCC6)C4)C3)cc2F)C(=O)N1] |
| 34 | 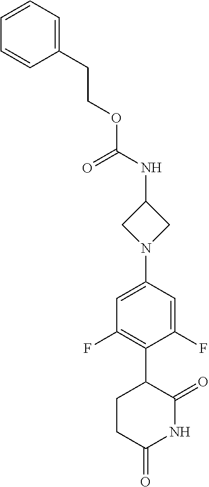 [SMILES: O=C1CCC(c2c(F)cc(N3CC(NC(=O)OCCc4ccccc4)C3)cc2F)C(=O)N1] |
| 35 | 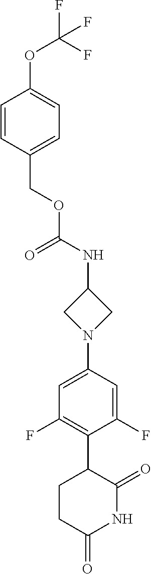 [SMILES: O=C1CCC(c2c(F)cc(N3CC(NC(=O)OCc4ccc(OC(F)(F)F)cc4)C3)cc2F)C(=O)N1] |
| 36 | 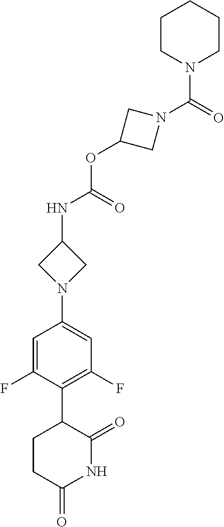 [SMILES: O=C1CCC(c2c(F)cc(N3CC(NC(=O)OC4CN(C(=O)N5CCCCC5)C4)C3)cc2F)C(=O)N1] |
| 37 | 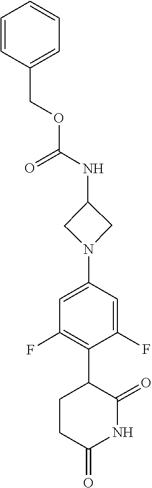 [SMILES: O=C1CCC(c2c(F)cc(N3CC(NC(=O)OCc4ccccc4)C3)cc2F)C(=O)N1] |
| 38 | 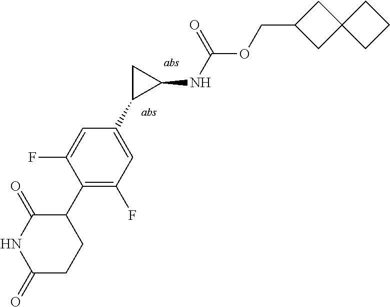 [SMILES: O=C1CCC(c2c(F)cc([C@@H]3C[C@H]3NC(=O)OCC3CC4(CCC4)C3)cc2F)C(=O)N1] |
| 39 | 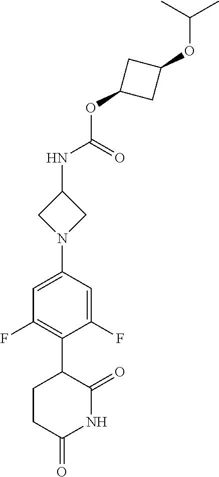 [SMILES: CC(C)O[C@H]1C[C@@H](OC(=O)NC2CN(c3cc(F)c(C4CCC(=O)NC4=O)c(F)c3)C2)C1] |
| 40 | 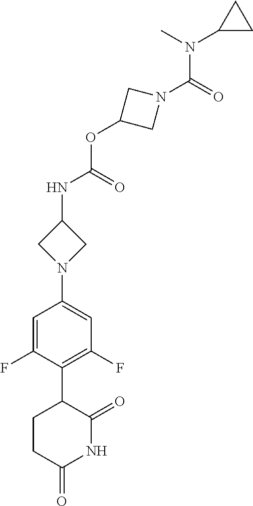 [SMILES: CN(C(=O)N1CC(OC(=O)NC2CN(c3cc(F)c(C4CCC(=O)NC4=O)c(F)c3)C2)C1)C1CC1] |
| 41 | 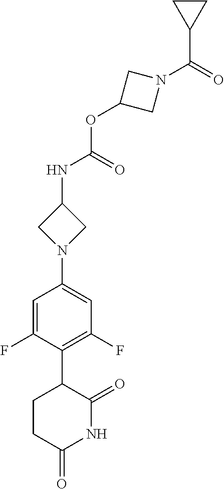 [SMILES: O=C1CCC(c2c(F)cc(N3CC(NC(=O)OC4CN(C(=O)C5CC5)C4)C3)cc2F)C(=O)N1] |
| 42 | 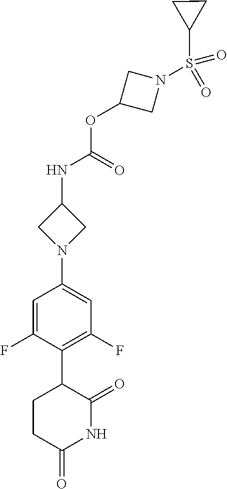 [SMILES: O=C1CCC(c2c(F)cc(N3CC(NC(=O)OC4CN(S(=O)(=O)C5CC5)C4)C3)cc2F)C(=O)N1] |
| 43 | 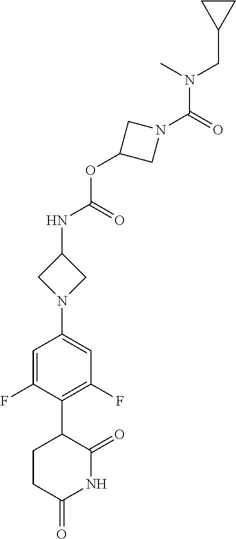 [SMILES: CN(CC1CC1)C(=O)N1CC(OC(=O)NC2CN(c3cc(F)c(C4CCC(=O)NC4=O)c(F)c3)C2)C1] |
| 44 | 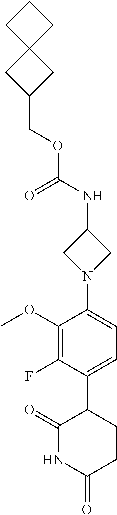 [SMILES: COc1c(N2CC(NC(=O)OCC3CC4(CCC4)C3)C2)ccc(C2CCC(=O)NC2=O)c1F] |
| 45 | 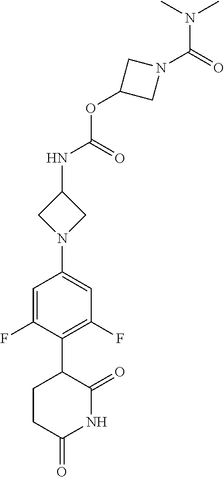 [SMILES: CN(C)C(=O)N1CC(OC(=O)NC2CN(c3cc(F)c(C4CCC(=O)NC4=O)c(F)c3)C2)C1] |
| 46 | 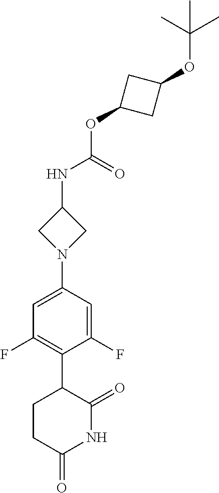 [SMILES: CC(C)(C)O[C@H]1C[C@@H](OC(=O)NC2CN(c3cc(F)c(C4CCC(=O)NC4=O)c(F)c3)C2)C1] |
| 47 | 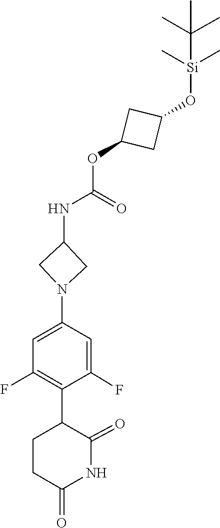 [SMILES: CC(C)(C)[Si](C)(C)O[C@H]1C[C@H](OC(=O)NC2CN(c3cc(F)c(C4CCC(=O)NC4=O)c(F)c3)C2)C1] |
| 48 | 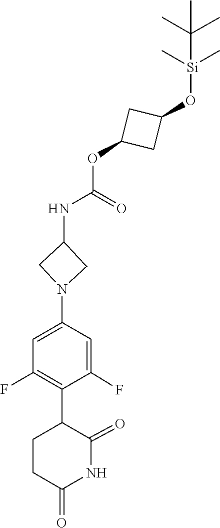 [SMILES: CC(C)(C)[Si](C)(C)O[C@H]1C[C@@H](OC(=O)NC2CN(c3cc(F)c(C4CCC(=O)NC4=O)c(F)c3)C2)C1] |
| 49 | 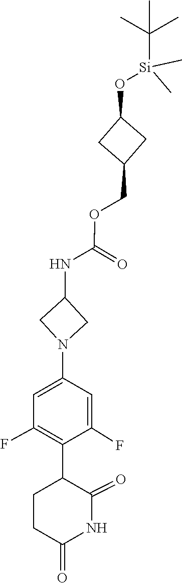 [SMILES: CC(C)(C)[Si](C)(C)O[C@H]1C[C@@H](COC(=O)NC2CN(c3cc(F)c(C4CCC(=O)NC4=O)c(F)c3)C2)C1] |
| 50 | 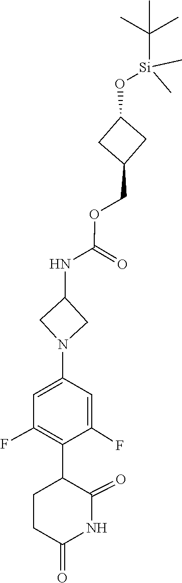 [SMILES: CC(C)(C)[Si](C)(C)O[C@H]1C[C@H](COC(=O)NC2CN(c3cc(F)c(C4CCC(=O)NC4=O)c(F)c3)C2)C1] |
| 51 | 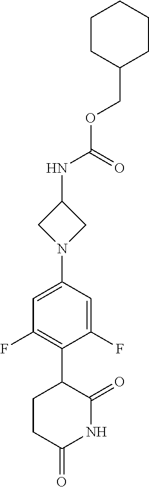 [SMILES: O=C1CCC(c2c(F)cc(N3CC(NC(=O)OCC4CCCCC4)C3)cc2F)C(=O)N1] |
| 52 | 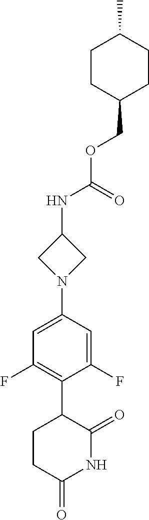 [SMILES: C[C@H]1CC[C@H](COC(=O)NC2CN(c3cc(F)c(C4CCC(=O)NC4=O)c(F)c3)C2)CC1] |
| 53 | 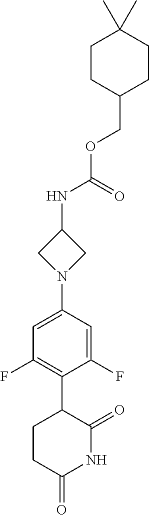 [SMILES: CC1(C)CCC(COC(=O)NC2CN(c3cc(F)c(C4CCC(=O)NC4=O)c(F)c3)C2)CC1] |
| 54 | 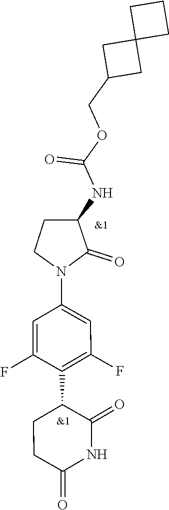 [SMILES: O=C1CC[C@H](c2c(F)cc(N3CC[C@@H](NC(=O)OCC4CC5(CCC5)C4)C3=O)cc2F)C(=O)N1] |
| 55 | 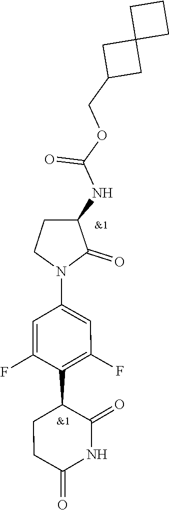 [SMILES: O=C1CC[C@@H](c2c(F)cc(N3CC[C@@H](NC(=O)OCC4CC5(CCC5)C4)C3=O)cc2F)C(=O)N1] |
| 56 | 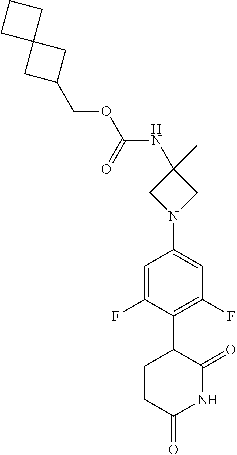 [SMILES: CC1(NC(=O)OCC2CC3(CCC3)C2)CN(c2cc(F)c(C3CCC(=O)NC3=O)c(F)c2)C1] |
| 57 | 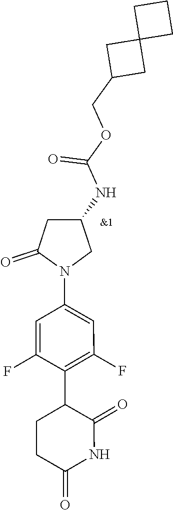 [SMILES: O=C1CCC(c2c(F)cc(N3C[C@@H](NC(=O)OCC4CC5(CCC5)C4)CC3=O)cc2F)C(=O)N1] |
| 58 | 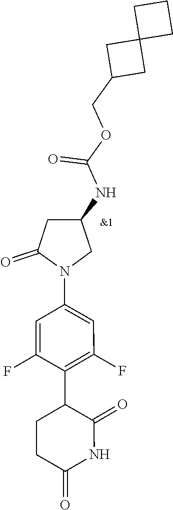 [SMILES: O=C1CCC(c2c(F)cc(N3C[C@H](NC(=O)OCC4CC5(CCC5)C4)CC3=O)cc2F)C(=O)N1] |
| 59 | 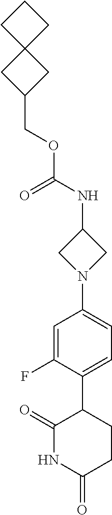 [SMILES: O=C1CCC(c2ccc(N3CC(NC(=O)OCC4CC5(CCC5)C4)C3)cc2F)C(=O)N1] |
| 60 | 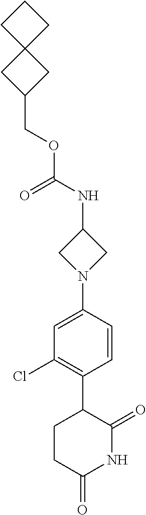 [SMILES: O=C1CCC(c2ccc(N3CC(NC(=O)OCC4CC5(CCC5)C4)C3)cc2Cl)C(=O)N1] |
| 61 | 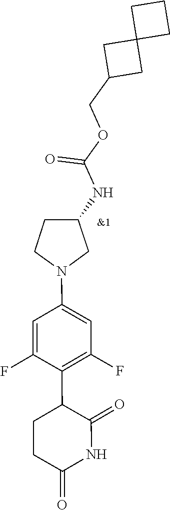 [SMILES: O=C1CCC(c2c(F)cc(N3CC[C@H](NC(=O)OCC4CC5(CCC5)C4)C3)cc2F)C(=O)N1] |
| 62 | 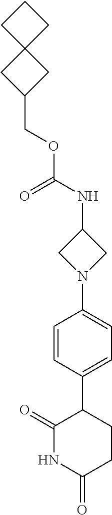 [SMILES: O=C1CCC(c2ccc(N3CC(NC(=O)OCC4CC5(CCC5)C4)C3)cc2)C(=O)N1] |
| 63 | 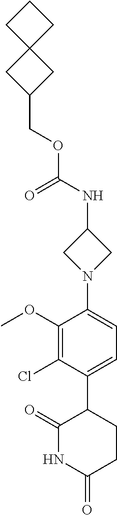 [SMILES: COc1c(N2CC(NC(=O)OCC3CC4(CCC4)C3)C2)ccc(C2CCC(=O)NC2=O)c1Cl] |
| 64 | 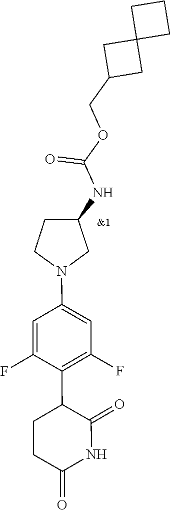 [SMILES: O=C1CCC(c2c(F)cc(N3CC[C@@H](NC(=O)OCC4CC5(CCC5)C4)C3)cc2F)C(=O)N1] |
| 65 | 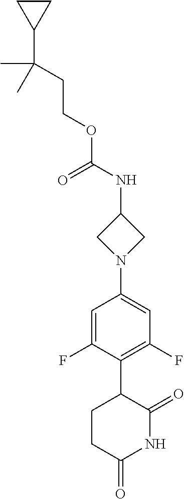 [SMILES: CC(C)(CCOC(=O)NC1CN(c2cc(F)c(C3CCC(=O)NC3=O)c(F)c2)C1)C1CC1] |
| 66 | 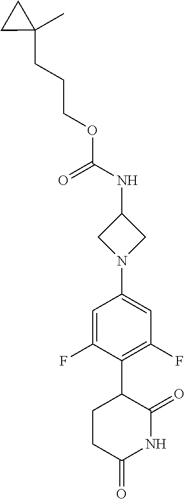 [SMILES: CC1(CCCOC(=O)NC2CN(c3cc(F)c(C4CCC(=O)NC4=O)c(F)c3)C2)CC1] |
| 67 | 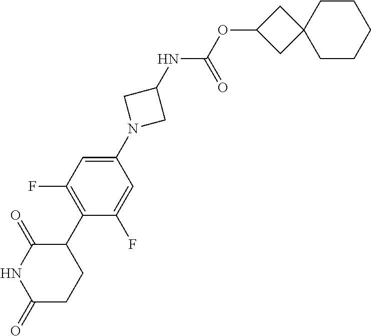 [SMILES: O=C1CCC(c2c(F)cc(N3CC(NC(=O)OC4CC5(CCCCC5)C4)C3)cc2F)C(=O)N1] |
| 68 | 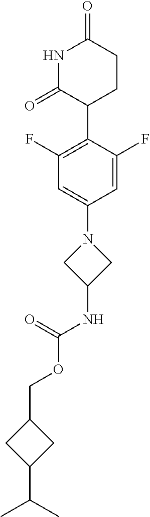 [SMILES: CC(C)C1CC(COC(=O)NC2CN(c3cc(F)c(C4CCC(=O)NC4=O)c(F)c3)C2)C1] |
| 69 | 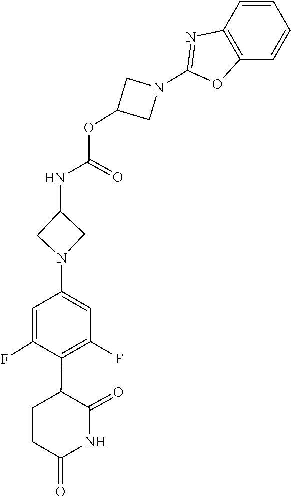 [SMILES: O=C1CCC(c2c(F)cc(N3CC(NC(=O)OC4CN(c5nc6ccccc6o5)C4)C3)cc2F)C(=O)N1] |
| 70 | 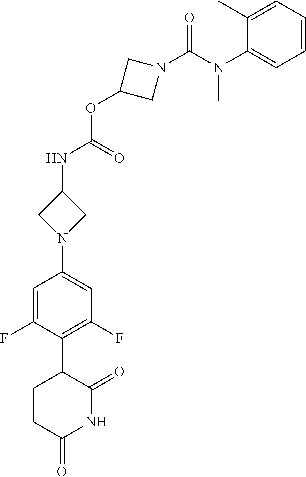 [SMILES: Cc1ccccc1N(C)C(=O)N1CC(OC(=O)NC2CN(c3cc(F)c(C4CCC(=O)NC4=O)c(F)c3)C2)C1] |
| 71 | 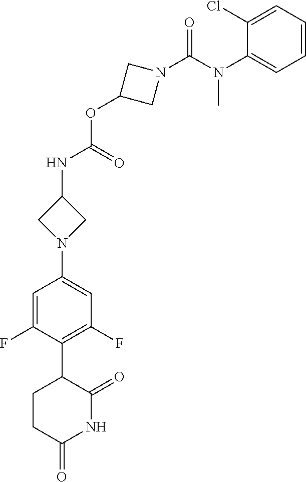 [SMILES: CN(C(=O)N1CC(OC(=O)NC2CN(c3cc(F)c(C4CCC(=O)NC4=O)c(F)c3)C2)C1)c1ccccc1Cl] |
| 72 | 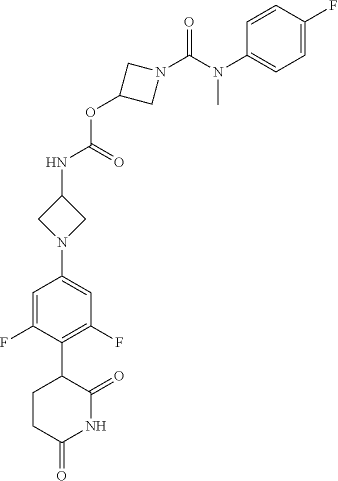 [SMILES: CN(C(=O)N1CC(OC(=O)NC2CN(c3cc(F)c(C4CCC(=O)NC4=O)c(F)c3)C2)C1)c1ccc(F)cc1] |
| 73 | 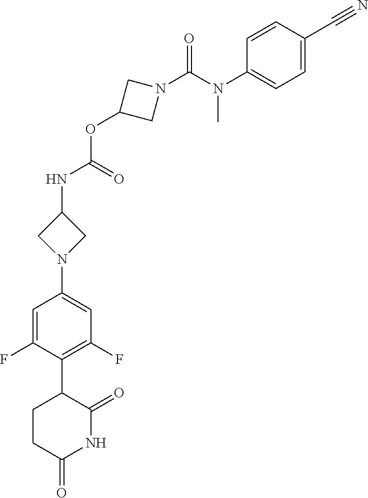 [SMILES: CN(C(=O)N1CC(OC(=O)NC2CN(c3cc(F)c(C4CCC(=O)NC4=O)c(F)c3)C2)C1)c1ccc(C#N)cc1] |
| 74 | 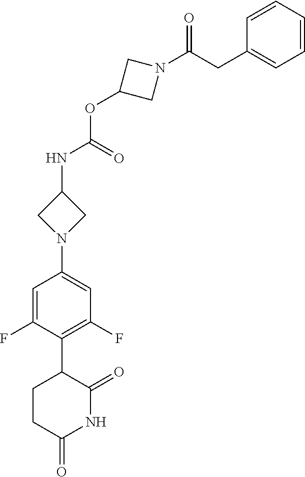 [SMILES: O=C1CCC(c2c(F)cc(N3CC(NC(=O)OC4CN(C(=O)Cc5ccccc5)C4)C3)cc2F)C(=O)N1] |
| 75 | 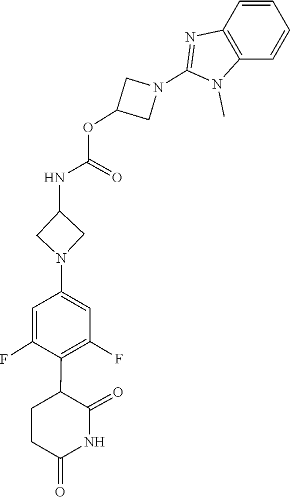 [SMILES: Cn1c(N2CC(OC(=O)NC3CN(c4cc(F)c(C5CCC(=O)NC5=O)c(F)c4)C3)C2)nc2ccccc21] |
| 76 | 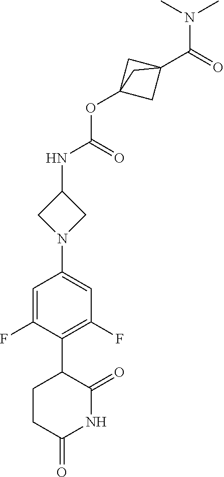 [SMILES: CN(C)C(=O)C12CC(OC(=O)NC3CN(c4cc(F)c(C5CCC(=O)NC5=O)c(F)c4)C3)(C1)C2] |
| 77 | 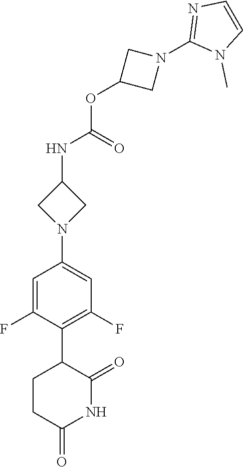 [SMILES: Cn1ccnc1N1CC(OC(=O)NC2CN(c3cc(F)c(C4CCC(=O)NC4=O)c(F)c3)C2)C1] |
| 78 | 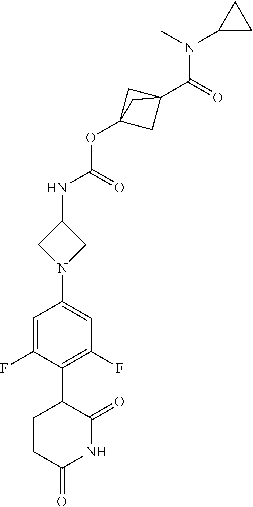 [SMILES: CN(C(=O)C12CC(OC(=O)NC3CN(c4cc(F)c(C5CCC(=O)NC5=O)c(F)c4)C3)(C1)C2)C1CC1] |
| 79 | 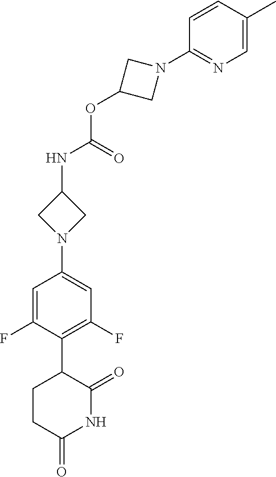 [SMILES: Cc1ccc(N2CC(OC(=O)NC3CN(c4cc(F)c(C5CCC(=O)NC5=O)c(F)c4)C3)C2)nc1] |
| 80 | 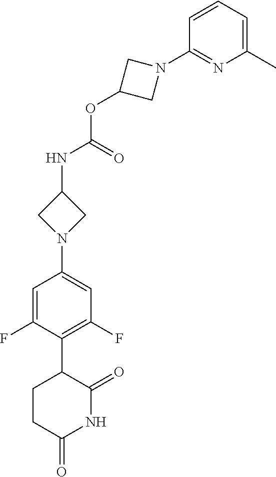 [SMILES: Cc1cccc(N2CC(OC(=O)NC3CN(c4cc(F)c(C5CCC(=O)NC5=O)c(F)c4)C3)C2)n1] |
| 81 | 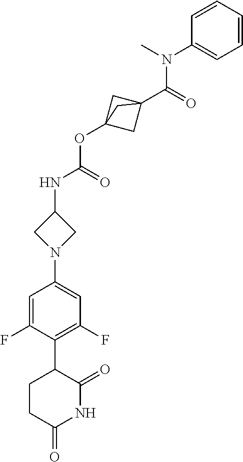 [SMILES: CN(C(=O)C12CC(OC(=O)NC3CN(c4cc(F)c(C5CCC(=O)NC5=O)c(F)c4)C3)(C1)C2)c1ccccc1] |
| 82 | 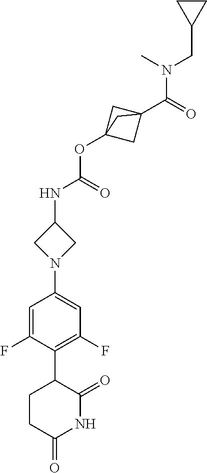 [SMILES: CN(CC1CC1)C(=O)C12CC(OC(=O)NC3CN(c4cc(F)c(C5CCC(=O)NC5=O)c(F)c4)C3)(C1)C2] |
| 83 | 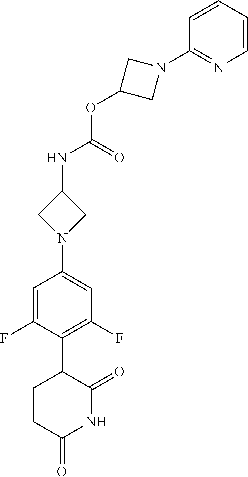 [SMILES: O=C1CCC(c2c(F)cc(N3CC(NC(=O)OC4CN(c5ccccn5)C4)C3)cc2F)C(=O)N1] |
| 84 | 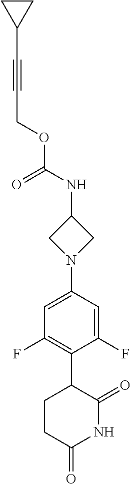 [SMILES: O=C1CCC(c2c(F)cc(N3CC(NC(=O)OCC#CC4CC4)C3)cc2F)C(=O)N1] |
| 85 | 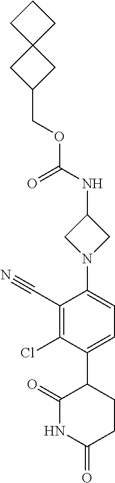 [SMILES: N#Cc1c(N2CC(NC(=O)OCC3CC4(CCC4)C3)C2)ccc(C2CCC(=O)NC2=O)c1Cl] |
| 86 | 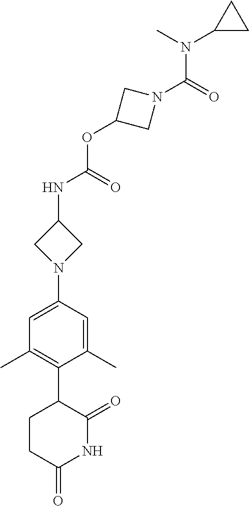 [SMILES: Cc1cc(N2CC(NC(=O)OC3CN(C(=O)N(C)C4CC4)C3)C2)cc(C)c1C1CCC(=O)NC1=O] |
| 87 | 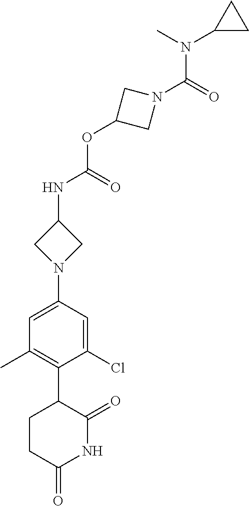 [SMILES: Cc1cc(N2CC(NC(=O)OC3CN(C(=O)N(C)C4CC4)C3)C2)cc(Cl)c1C1CCC(=O)NC1=O] |
| 88 | 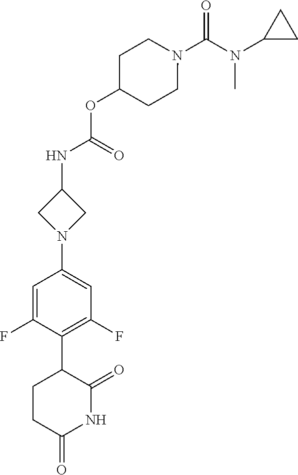 [SMILES: CN(C(=O)N1CCC(OC(=O)NC2CN(c3cc(F)c(C4CCC(=O)NC4=O)c(F)c3)C2)CC1)C1CC1] |
| 89 | 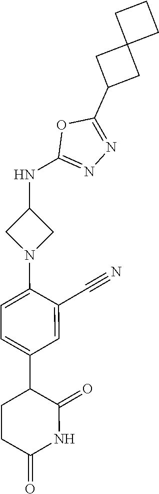 [SMILES: N#Cc1cc(C2CCC(=O)NC2=O)ccc1N1CC(Nc2nnc(C3CC4(CCC4)C3)o2)C1] |
| 90 | 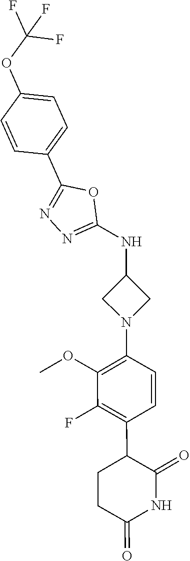 [SMILES: COc1c(N2CC(Nc3nnc(-c4ccc(OC(F)(F)F)cc4)o3)C2)ccc(C2CCC(=O)NC2=O)c1F] |
| 91 | 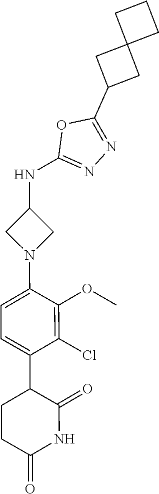 [SMILES: COc1c(N2CC(Nc3nnc(C4CC5(CCC5)C4)o3)C2)ccc(C2CCC(=O)NC2=O)c1Cl] |
| 92 | 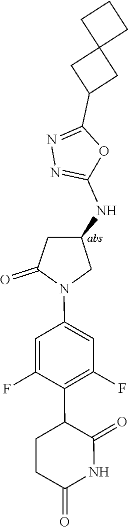 [SMILES: O=C1CCC(c2c(F)cc(N3C[C@H](Nc4nnc(C5CC6(CCC6)C5)o4)CC3=O)cc2F)C(=O)N1] |
| 93 | 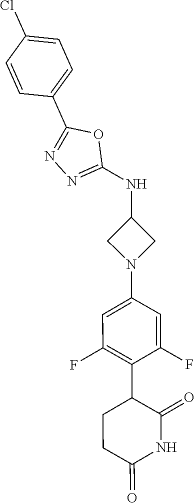 [SMILES: O=C1CCC(c2c(F)cc(N3CC(Nc4nnc(-c5ccc(Cl)cc5)o4)C3)cc2F)C(=O)N1] |
| 94 | 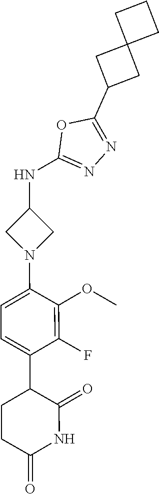 [SMILES: COc1c(N2CC(Nc3nnc(C4CC5(CCC5)C4)o3)C2)ccc(C2CCC(=O)NC2=O)c1F] |
| 95 | 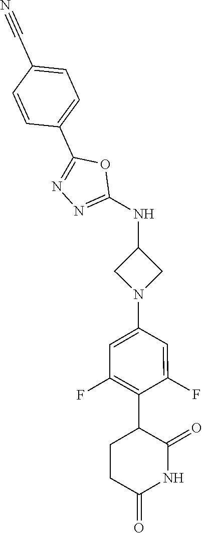 [SMILES: N#Cc1ccc(-c2nnc(NC3CN(c4cc(F)c(C5CCC(=O)NC5=O)c(F)c4)C3)o2)cc1] |
| 96 | 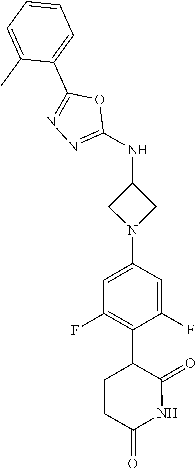 [SMILES: Cc1ccccc1-c1nnc(NC2CN(c3cc(F)c(C4CCC(=O)NC4=O)c(F)c3)C2)o1] |
| 97 | 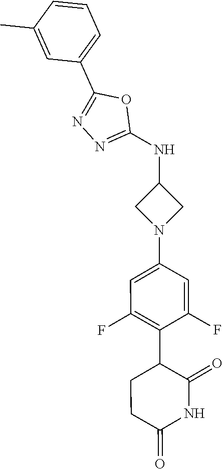 [SMILES: Cc1cccc(-c2nnc(NC3CN(c4cc(F)c(C5CCC(=O)NC5=O)c(F)c4)C3)o2)c1] |
| 98 | 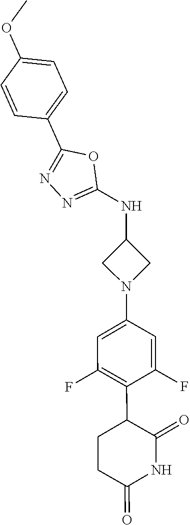 [SMILES: COc1ccc(-c2nnc(NC3CN(c4cc(F)c(C5CCC(=O)NC5=O)c(F)c4)C3)o2)cc1] |
| 99 | 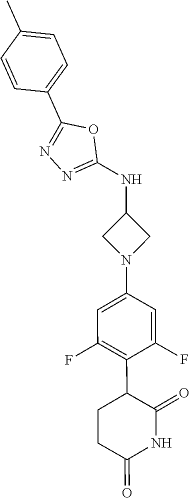 [SMILES: Cc1ccc(-c2nnc(NC3CN(c4cc(F)c(C5CCC(=O)NC5=O)c(F)c4)C3)o2)cc1] |
| 100 | 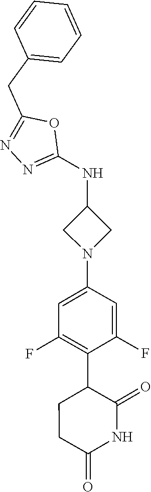 [SMILES: O=C1CCC(c2c(F)cc(N3CC(Nc4nnc(Cc5ccccc5)o4)C3)cc2F)C(=O)N1] |
| 101 | 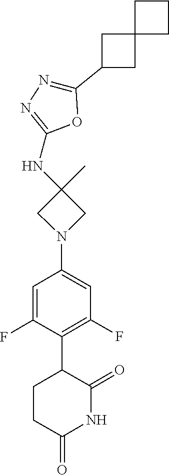 [SMILES: CC1(Nc2nnc(C3CC4(CCC4)C3)o2)CN(c2cc(F)c(C3CCC(=O)NC3=O)c(F)c2)C1] |
| 102 | 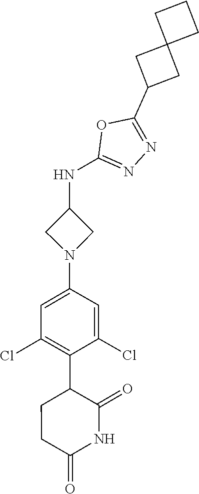 [SMILES: O=C1CCC(c2c(Cl)cc(N3CC(Nc4nnc(C5CC6(CCC6)C5)o4)C3)cc2Cl)C(=O)N1] |
| 103 | 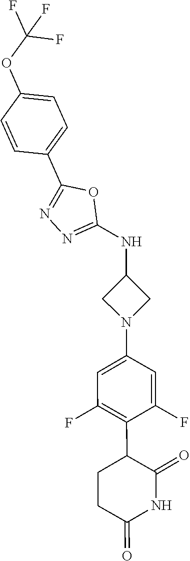 [SMILES: O=C1CCC(c2c(F)cc(N3CC(Nc4nnc(-c5ccc(OC(F)(F)F)cc5)o4)C3)cc2F)C(=O)N1] |
| 104 | 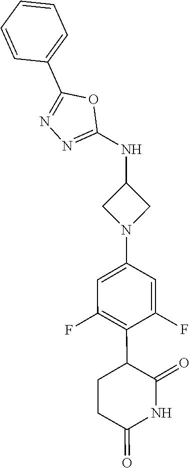 [SMILES: O=C1CCC(c2c(F)cc(N3CC(Nc4nnc(-c5ccccc5)o4)C3)cc2F)C(=O)N1] |
| 105 | 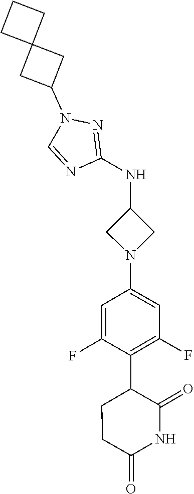 [SMILES: O=C1CCC(c2c(F)cc(N3CC(Nc4ncn(C5CC6(CCC6)C5)n4)C3)cc2F)C(=O)N1] |
| 106 | 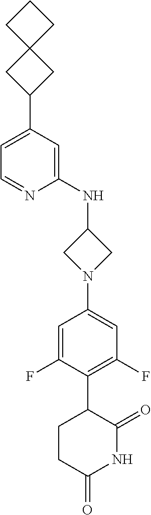 [SMILES: O=C1CCC(c2c(F)cc(N3CC(Nc4cc(C5CC6(CCC6)C5)ccn4)C3)cc2F)C(=O)N1] |
| 107 | 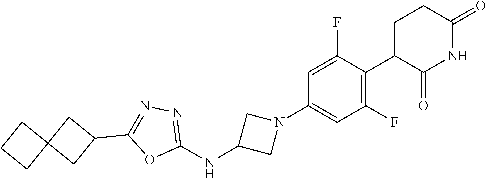 [SMILES: O=C1CCC(c2c(F)cc(N3CC(Nc4nnc(C5CC6(CCC6)C5)o4)C3)cc2F)C(=O)N1] |
| 108 | 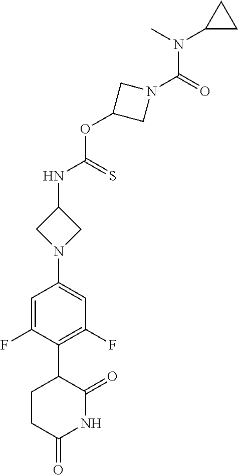 [SMILES: CN(C(=O)N1CC(OC(=S)NC2CN(c3cc(F)c(C4CCC(=O)NC4=O)c(F)c3)C2)C1)C1CC1] |
| 109 | 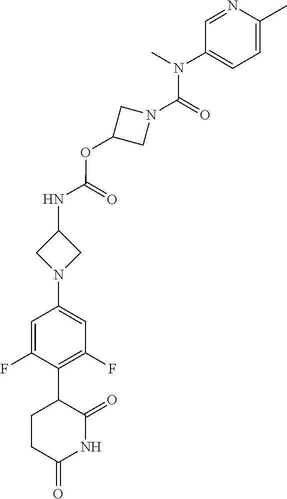 [SMILES: Cc1ccc(N(C)C(=O)N2CC(OC(=O)NC3CN(c4cc(F)c(C5CCC(=O)NC5=O)c(F)c4)C3)C2)cn1] |
| 110 | 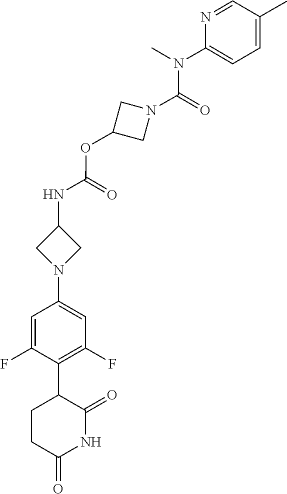 [SMILES: Cc1ccc(N(C)C(=O)N2CC(OC(=O)NC3CN(c4cc(F)c(C5CCC(=O)NC5=O)c(F)c4)C3)C2)nc1] |
| 111 | 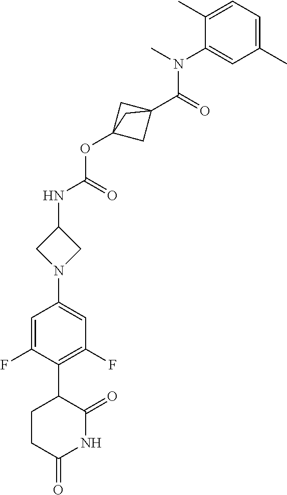 [SMILES: Cc1ccc(C)c(N(C)C(=O)C23CC(OC(=O)NC4CN(c5cc(F)c(C6CCC(=O)NC6=O)c(F)c5)C4)(C2)C3)c1] |
| 112 | 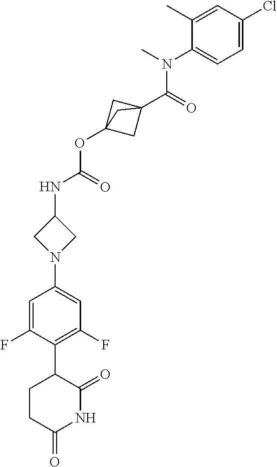 [SMILES: Cc1cc(Cl)ccc1N(C)C(=O)C12CC(OC(=O)NC3CN(c4cc(F)c(C5CCC(=O)NC5=O)c(F)c4)C3)(C1)C2] |
| 113 | 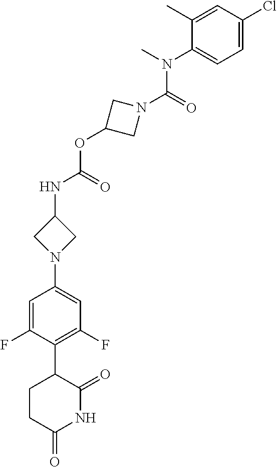 [SMILES: Cc1cc(Cl)ccc1N(C)C(=O)N1CC(OC(=O)NC2CN(c3cc(F)c(C4CCC(=O)NC4=O)c(F)c3)C2)C1] |
| 114 | 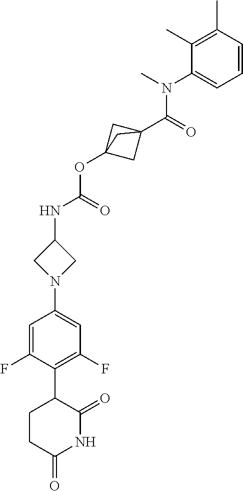 [SMILES: Cc1cccc(N(C)C(=O)C23CC(OC(=O)NC4CN(c5cc(F)c(C6CCC(=O)NC6=O)c(F)c5)C4)(C2)C3)c1C] |
| 115 | 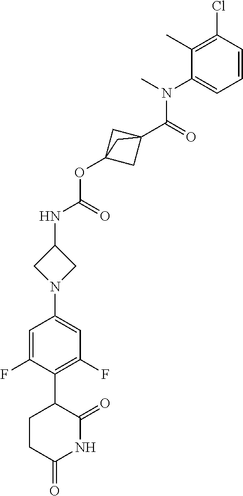 [SMILES: Cc1c(Cl)cccc1N(C)C(=O)C12CC(OC(=O)NC3CN(c4cc(F)c(C5CCC(=O)NC5=O)c(F)c4)C3)(C1)C2] |
| 116 | 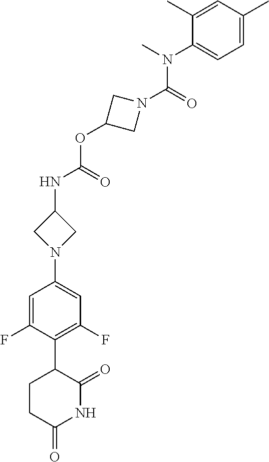 [SMILES: Cc1ccc(N(C)C(=O)N2CC(OC(=O)NC3CN(c4cc(F)c(C5CCC(=O)NC5=O)c(F)c4)C3)C2)c(C)c1] |
| 117 | 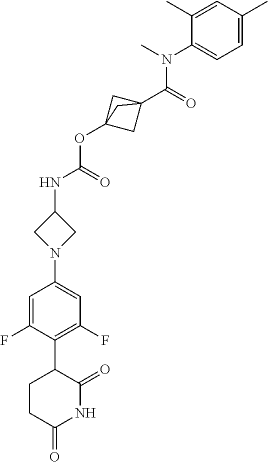 [SMILES: Cc1ccc(N(C)C(=O)C23CC(OC(=O)NC4CN(c5cc(F)c(C6CCC(=O)NC6=O)c(F)c5)C4)(C2)C3)c(C)c1] |
| 118 | 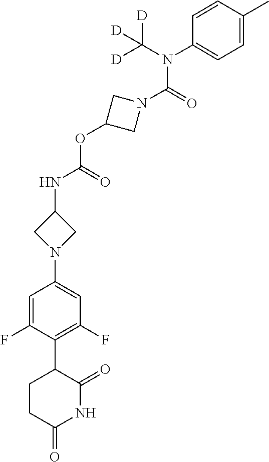 [SMILES: [2H]C([2H])([2H])N(C(=O)N1CC(OC(=O)NC2CN(c3cc(F)c(C4CCC(=O)NC4=O)c(F)c3)C2)C1)c1ccc(C)cc1] |
| 119 | 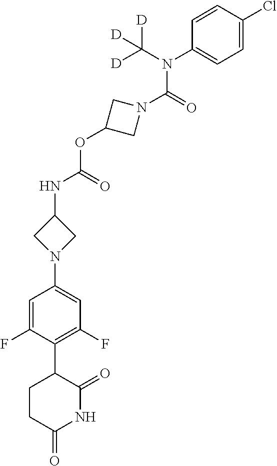 [SMILES: [2H]C([2H])([2H])N(C(=O)N1CC(OC(=O)NC2CN(c3cc(F)c(C4CCC(=O)NC4=O)c(F)c3)C2)C1)c1ccc(Cl)cc1] |
| 120 | 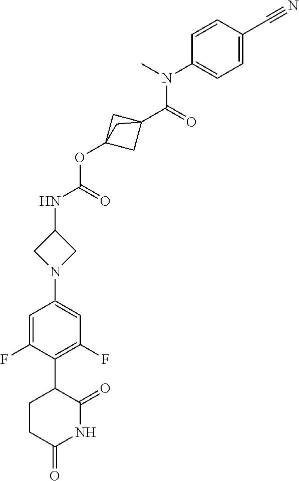 [SMILES: CN(C(=O)C12CC(OC(=O)NC3CN(c4cc(F)c(C5CCC(=O)NC5=O)c(F)c4)C3)(C1)C2)c1ccc(C#N)cc1] |
| 121 | 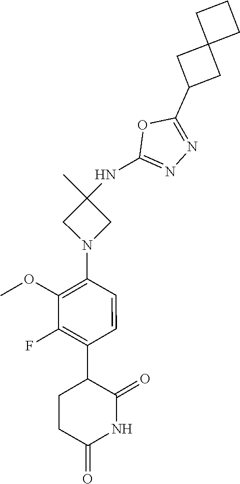 [SMILES: COc1c(N2CC(C)(Nc3nnc(C4CC5(CCC5)C4)o3)C2)ccc(C2CCC(=O)NC2=O)c1F] |
| 122 | 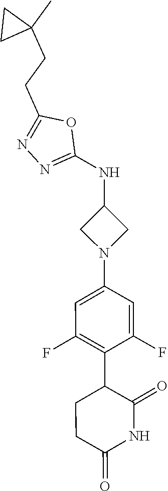 [SMILES: CC1(CCc2nnc(NC3CN(c4cc(F)c(C5CCC(=O)NC5=O)c(F)c4)C3)o2)CC1] |
| 123 | 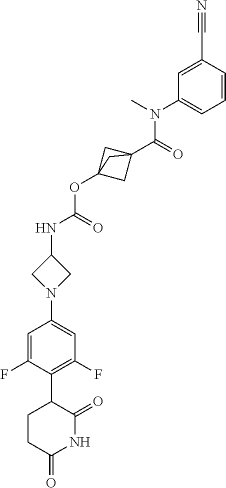 [SMILES: CN(C(=O)C12CC(OC(=O)NC3CN(c4cc(F)c(C5CCC(=O)NC5=O)c(F)c4)C3)(C1)C2)c1cccc(C#N)c1] |
| 124 | 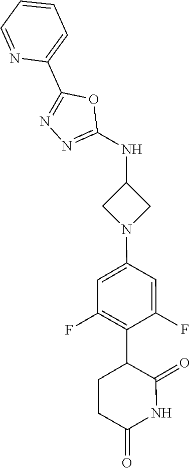 [SMILES: O=C1CCC(c2c(F)cc(N3CC(Nc4nnc(-c5ccccn5)o4)C3)cc2F)C(=O)N1] |
| 125 | 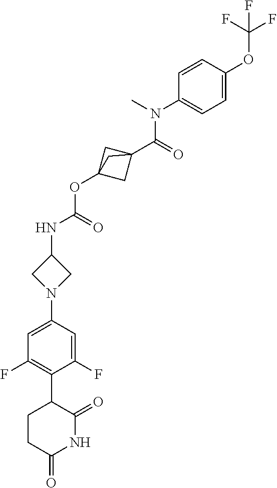 [SMILES: CN(C(=O)C12CC(OC(=O)NC3CN(c4cc(F)c(C5CCC(=O)NC5=O)c(F)c4)C3)(C1)C2)c1ccc(OC(F)(F)F)cc1] |
| 126 | 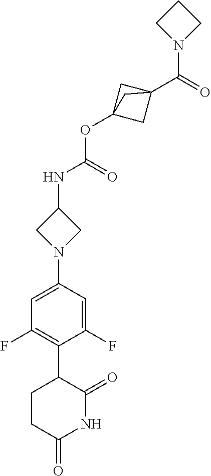 [SMILES: O=C1CCC(c2c(F)cc(N3CC(NC(=O)OC45CC(C(=O)N6CCC6)(C4)C5)C3)cc2F)C(=O)N1] |
| 127 | 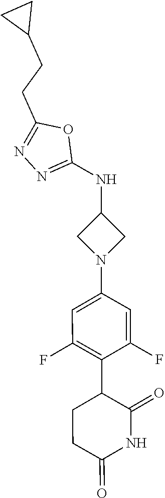 [SMILES: O=C1CCC(c2c(F)cc(N3CC(Nc4nnc(CCC5CC5)o4)C3)cc2F)C(=O)N1] |
| 128 | 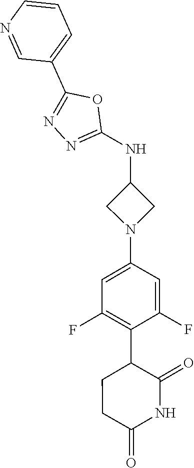 [SMILES: O=C1CCC(c2c(F)cc(N3CC(Nc4nnc(-c5cccnc5)o4)C3)cc2F)C(=O)N1] |
| 129 | 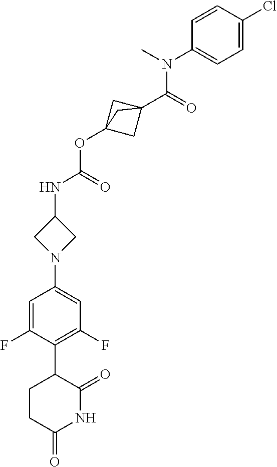 [SMILES: CN(C(=O)C12CC(OC(=O)NC3CN(c4cc(F)c(C5CCC(=O)NC5=O)c(F)c4)C3)(C1)C2)c1ccc(Cl)cc1] |
| 130 | 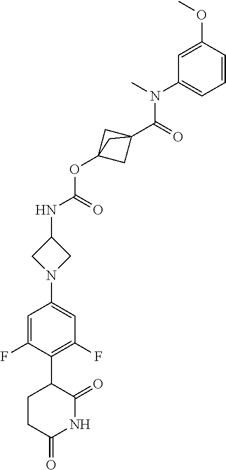 [SMILES: COc1cccc(N(C)C(=O)C23CC(OC(=O)NC4CN(c5cc(F)c(C6CCC(=O)NC6=O)c(F)c5)C4)(C2)C3)c1] |
| 131 | 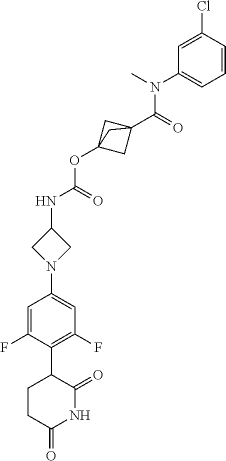 [SMILES: CN(C(=O)C12CC(OC(=O)NC3CN(c4cc(F)c(C5CCC(=O)NC5=O)c(F)c4)C3)(C1)C2)c1cccc(Cl)c1] |
| 132 | 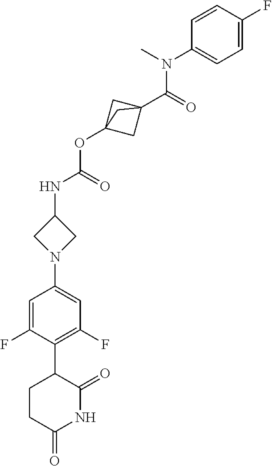 [SMILES: CN(C(=O)C12CC(OC(=O)NC3CN(c4cc(F)c(C5CCC(=O)NC5=O)c(F)c4)C3)(C1)C2)c1ccc(F)cc1] |
| 133 | 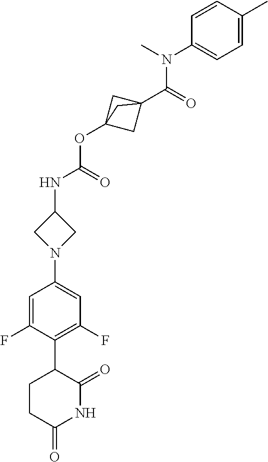 [SMILES: Cc1ccc(N(C)C(=O)C23CC(OC(=O)NC4CN(c5cc(F)c(C6CCC(=O)NC6=O)c(F)c5)C4)(C2)C3)cc1] |
| 134 | 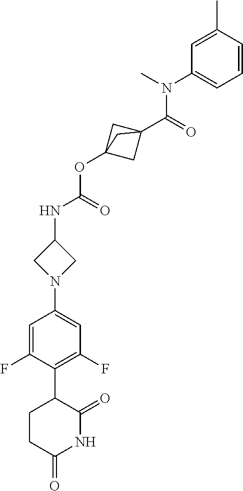 [SMILES: Cc1cccc(N(C)C(=O)C23CC(OC(=O)NC4CN(c5cc(F)c(C6CCC(=O)NC6=O)c(F)c5)C4)(C2)C3)c1] |
| 135 | 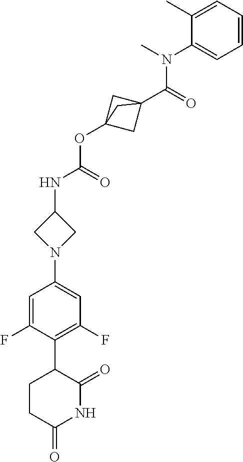 [SMILES: Cc1ccccc1N(C)C(=O)C12CC(OC(=O)NC3CN(c4cc(F)c(C5CCC(=O)NC5=O)c(F)c4)C3)(C1)C2] |
| 136 | 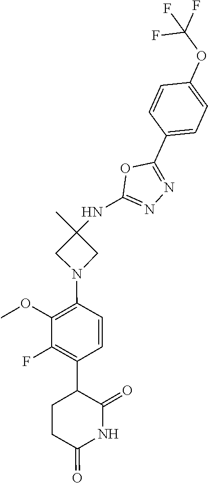 [SMILES: COc1c(N2CC(C)(Nc3nnc(-c4ccc(OC(F)(F)F)cc4)o3)C2)ccc(C2CCC(=O)NC2=O)c1F] |
| 137 | 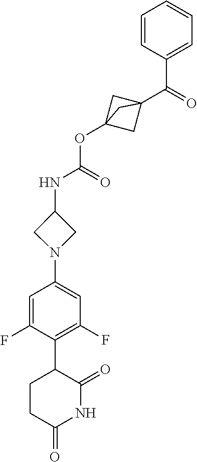 [SMILES: O=C1CCC(c2c(F)cc(N3CC(NC(=O)OC45CC(C(=O)c6ccccc6)(C4)C5)C3)cc2F)C(=O)N1] |
| 138 | 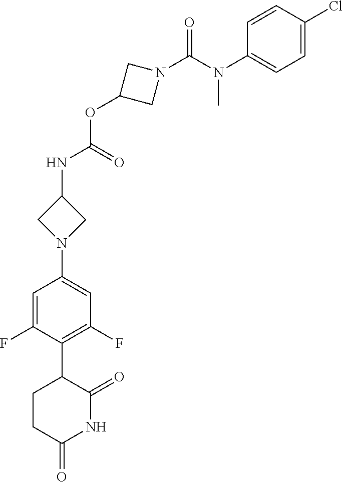 [SMILES: CN(C(=O)N1CC(OC(=O)NC2CN(c3cc(F)c(C4CCC(=O)NC4=O)c(F)c3)C2)C1)c1ccc(Cl)cc1] |
| 139 | 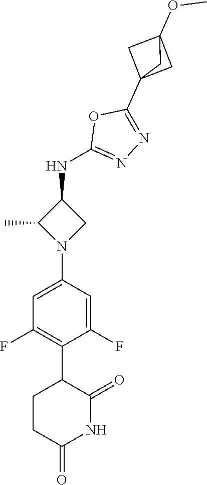 [SMILES: COC12CC(c3nnc(N[C@H]4CN(c5cc(F)c(C6CCC(=O)NC6=O)c(F)c5)[C@@H]4C)o3)(C1)C2] |
| 140 | 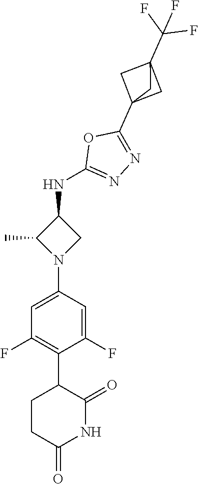 [SMILES: C[C@@H]1[C@@H](Nc2nnc(C34CC(C(F)(F)F)(C3)C4)o2)CN1c1cc(F)c(C2CCC(=O)NC2=O)c(F)c1] |
| 141 | 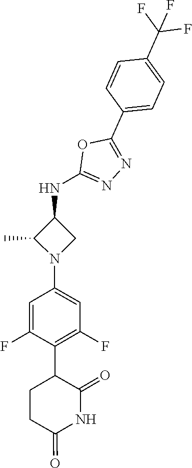 [SMILES: C[C@@H]1[C@@H](Nc2nnc(-c3ccc(C(F)(F)F)cc3)o2)CN1c1cc(F)c(C2CCC(=O)NC2=O)c(F)c1] |
| 142 | 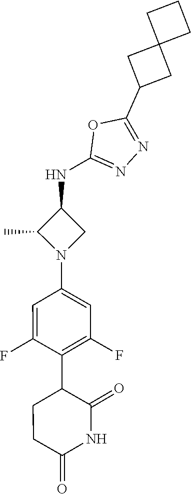 [SMILES: C[C@@H]1[C@@H](Nc2nnc(C3CC4(CCC4)C3)o2)CN1c1cc(F)c(C2CCC(=O)NC2=O)c(F)c1] |
| 143 | 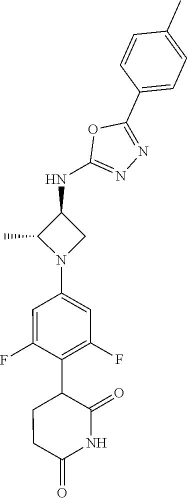 [SMILES: Cc1ccc(-c2nnc(N[C@H]3CN(c4cc(F)c(C5CCC(=O)NC5=O)c(F)c4)[C@@H]3C)o2)cc1] |
| 144 | 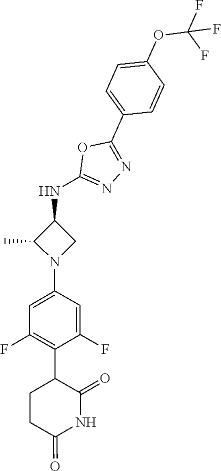 [SMILES: C[C@@H]1[C@@H](Nc2nnc(-c3ccc(OC(F)(F)F)cc3)o2)CN1c1cc(F)c(C2CCC(=O)NC2=O)c(F)c1] |

### Pharmaceutical Compositions

In another embodiment, the present disclosure provides a pharmaceutical composition comprising a compound described herein, or pharmaceutically acceptable salt thereof, and a pharmaceutically acceptable excipient. In certain embodiments, the pharmaceutical composition comprises an effective amount of the compound. In certain embodiments, the pharmaceutical composition comprises a therapeutically effective amount of the compound.

The pharmaceutical compositions provided herein can be administered by a variety of routes including, but not limited to, oral (enteral) administration, parenteral (by injection) administration, rectal administration, transdermal administration, intradermal administration, intrathecal administration, subcutaneous (SC) administration, intravenous (IV) administration, intramuscular (IM) administration, and intranasal administration.

Compositions for oral administration can take the form of bulk liquid solutions or suspensions, or bulk powders. In some embodiments, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the compound is usually a minor component with the remainder being various vehicles or excipients and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable excipients known in the art. As before, the active compound in such compositions is typically a minor component with the remainder being the injectable excipient and the like.

Transdermal compositions are typically formulated as a topical ointment or cream containing the active ingredient(s). When formulated as a ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or Formulation. All such known transdermal formulations and ingredients are included within the scope of the disclosure provided herein.

The compounds provided herein can also be administered by a transdermal device. Accordingly, transdermal administration can be accomplished using a patch either of the reservoir or porous membrane type, or of a solid matrix variety.

The above-described components for orally administrable, injectable or topically administrable compositions are merely representative. Other materials as well as processing techniques and the like are set forth in Part 8 of Remington's Pharmaceutical Sciences, 17th edition, 1985, Mack Publishing Company, Easton, Pennsylvania, which is incorporated herein by reference.

### Methods of Treatment and Uses

Furthermore, the compounds and pharmaceutical compositions described herein are contemplated as useful in the treatment or prevention of disorders in subjects in need thereof. Compounds described herein, in one embodiment, are used to degrade CDK2 for the treatment of prevention of a disorder.

Cyclin dependent kinases, or CDKs, are a family of closely related kinases that regulate progression through the cell cycle. CDK activity is further modulated by levels of specific cyclins, for example, cyclin E1 activates cyclin dependent kinase 2, or CDK2. Elimination of CDK2 is contemplated to treat patients bearing tumors with activated CDK2. Mechanisms activating CDK2 in tumors include, but are not limited to, amplification or high expression of Cyclin E1 or Cyclin E2.

Accordingly, in one embodiment of the disclosure, a compound, or pharmaceutically acceptable salt thereof, or pharmaceutical composition described herein is administered to a subject to degrade CDK2 in the subject.

In one aspect of the disclosure, described herein is a method of treating or preventing a disorder in a subject in need thereof, comprising administering to the subject an effective amount of a compound, or pharmaceutically acceptable salt thereof, or pharmaceutical composition described herein.

In another aspect, described herein is a method of degrading CDK2 in a subject suffering from a disorder, comprising administering to the subject a therapeutically effective amount of a compound described herein, or pharmaceutically acceptable salt thereof, or pharmaceutical composition described herein. In some embodiments, the compound binds to cereblon and a CDK2 protein to induce ubiquitination and subsequent proteasomal degradation of the CDK2.

Exemplary disorders that can be treated or prevented by the methods of the present disclosure include but are not limited to, cancer of the bladder, bone, brain, breast, cervix, chest, colon, endometrium, esophagus, eye, head, kidney, liver, lymph nodes, lung, upper aerodigestive tract (including nasal cavity and paranasal sinuses, nasopharynx or cavum, oral cavity, oropharynx, larynx, hypopharynx and salivary glands, neck, ovaries, pancreas, prostate, rectum, skin, stomach, testis, throat, or uterus. Other exemplary disorders include, but are not limited to, amyloidosis, neuroblastoma, meningioma, hemangiopericytoma, multiple brain metastase, glioblastoma multiforms, glioblastoma, brain stem glioma, poor prognosis malignant brain tumor, malignant glioma, recurrent malignant glioma, anaplastic astrocytoma, anaplastic oligodendroglioma, neuroendocrine tumor, e.g., neuroendocrine prostate cancer such as castration-resistant neuroendocrine prostate cancer (NEPC) and lung neuroendocrine tumors (Lu-NETs), rectal adenocarcinoma, colorectal cancer, including stage 3 and stage 4 colorectal cancer, unresectable colorectal carcinoma, metastatic hepatocellular carcinoma, Kaposi's sarcoma, malignant melanoma, malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, leiomyosarcoma, fibrodysplasia ossificans progressive, hormone refractory prostate cancer, resected high-risk soft tissue sarcoma, unresectable hepatocellular carcinoma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, and leiomyoma; and blood bourne (liquid) or hematological cancers, including but not limited to leukemias, lymphomas, and myelomas, such as diffuse large B-cell lymphoma (DLBCL), B-cell immunoblastic lymphoma, small non-cleaved cell lymphoma, human lymphotropic virus-type 1 (HTLV-1) leukemia/lymphoma, adult T-cell lymphoma, peripheral T-cell lymphoma (PTCL), cutaneous T-cell lymphoma (CTCL), mantle cell lymphoma (MCL), Hodgkin's lymphoma (HL), non-Hodgkin's lymphoma (NHL), AIDS-related lymphoma, follicular lymphoma, small lymphocytic lymphoma, T-cell/histiocyte rich large B-cell lymphoma, transformed lymphoma, primary mediastinal (thymic) large B-cell lymphoma, splenic marginal zone lymphoma, Richter's transformation, nodal marginal zone lymphoma, ALK-positive large B-cell lymphoma, indolent lymphoma (for example, DLBCL, follicular lymphoma, or marginal zone lymphoma), acute myelogenous leukemia (AML), acute lymphocytic leukemia (ALL), adult T-cell leukemia, chronic lymphocytic leukemia (CLL), small lymphocytic lymphoma (SLL), hairy cell leukemia, myelodysplasia, myeloproliferative disorders, chronic myelogenous leukemia (CML), acute monocytic leukemia (AMoL), myelodysplastic syndrome (MDS), human lymphotropic virus-type 1 (HTLV-1) leukemia, mastocytosis, B-cell acute lymphoblastic leukemia, Non-Hodgkin's Lymphoma, Hodgkin's Lymphoma, and multiple myeloma (MM).

In some embodiments, the disorder is breast cancer or ovarian cancer. In some embodiments, the breast cancer is estrogen receptor positive breast cancer or triple negative breast cancer.

In another aspect of the disclosure, described herein is a method of treating cancer (e.g., a cancer described herein) in a subject in need thereof, comprising administering to the subject a therapeutically effective amount of a compound described herein, or pharmaceutically acceptable salt thereof, or pharmaceutical composition described herein.

In another aspect, described herein is a method of degrading CDK2 in a subject suffering from cancer (e.g., a cancer described herein), comprising administering to the subject a therapeutically effective amount of a compound described herein, or pharmaceutically acceptable salt thereof, or pharmaceutical composition described herein.

In another aspect, described herein is a method of treating a solid tumor (e.g., a solid tumor described herein) in a subject in need thereof, comprising administering to the subject a therapeutically effective amount of a compound described herein, or pharmaceutically acceptable salt thereof, or pharmaceutical composition described herein.

In another aspect, described herein is a method of treating a liquid tumor (e.g., a liquid tumor described herein) in a subject in need thereof, comprising administering to the subject a therapeutically effective amount of a compound described herein, or pharmaceutically acceptable salt thereof, or pharmaceutical composition described herein. In some embodiments, the liquid tumor is that of a haematological cancer (e.g., a haematological cancer described herein).

### Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “alkyl” as used herein refers to a radical of a straight-chain or branched saturated hydrocarbon group. In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”, also referred to herein as “lower alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Common alkyl abbreviations include Me (—CH₃), Et (—CH₂CH₃), iPr (—CH(CH₃)₂), nPr (—CH₂CH₂CH₃), n-Bu (—CH₂CH₂CH₂CH₃), or i-Bu (—CH₂CH(CH₃)₂).

The term “alkenyl” as used herein refers to a radical of a straight-chain or branched hydrocarbon group having, one or more carbon-carbon double bonds. In some embodiments, an alkenyl group has 2 to 10 carbon atoms (“C2-10 alkenyl”). In some embodiments, an alkenyl group has 2 to 9 carbon atoms (“C2-9 alkenyl”). In some embodiments, an alkenyl group has 2 to 8 carbon atoms (“C2-8 alkenyl”). In some embodiments, an alkenyl group has 2 to 7 carbon atoms (“C2-7 alkenyl”). In some embodiments, an alkenyl group has 2 to 6 carbon atoms (“C2-6 alkenyl”). In some embodiments, an alkenyl group has 2 to 5 carbon atoms (“C2-5 alkenyl”). In some embodiments, an alkenyl group has 2 to 4 carbon atoms (“C2-4 alkenyl”). In some embodiments, an alkenyl group has 2 to 3 carbon atoms (“C2-3 alkenyl”). In some embodiments, an alkenyl group has 2 carbon atoms (“C2 alkenyl”). The one or more carbon-carbon double bonds can be internal (such as in 2-butenyl) or terminal (such as in 1-butenyl). Examples of C2-4 alkenyl groups include ethenyl (C2), 1-propenyl (C3), 2-propenyl (C3), 1-butenyl (C4), 2-butenyl (C4), butadienyl (C4), and the like. Examples of C2-6 alkenyl groups include the aforementioned C2-4 alkenyl groups as well as pentenyl (C5), pentadienyl (C5), hexenyl (C6), and the like. Additional examples of alkenyl include heptenyl (C7), octenyl (C8), octatrienyl (C8), and the like.

The term “alkynyl” as used herein refers to a radical of a straight-chain or branched hydrocarbon group having one or more carbon-carbon triple bonds (e.g., 1, 2, 3, or 4 carbon-carbon triple bonds). In some embodiments, an alkynyl group has 2 to 10 carbon atoms (“C2-10 alkynyl”). In some embodiments, an alkynyl group has 2 to 9 carbon atoms (“C2-9 alkynyl”). In some embodiments, an alkynyl group has 2 to 8 carbon atoms (“C2-8 alkynyl”). In some embodiments, an alkynyl group has 2 to 7 carbon atoms (“C2-7 alkynyl”). In some embodiments, an alkynyl group has 2 to 6 carbon atoms (“C2-6 alkynyl”). In some embodiments, an alkynyl group has 2 to 5 carbon atoms (“C2-5 alkynyl”). In some embodiments, an alkynyl group has 2 to 4 carbon atoms (“C2-4 alkynyl”). In some embodiments, an alkynyl group has 2 to 3 carbon atoms (“C2-3 alkynyl”). In some embodiments, an alkynyl group has 2 carbon atoms (“C2 alkynyl”). The one or more carbon-carbon triple bonds can be internal (such as in 2-butynyl) or terminal (such as in 1-butynyl). Examples of C2-4 alkynyl groups include, without limitation, ethynyl (C2), 1-propynyl (C3), 2-propynyl (C3), 1-butynyl (C4), 2-butynyl (C4), and the like. Examples of C2-6 alkenyl groups include the aforementioned C2-4 alkynyl groups as well as pentynyl (C5), hexynyl (C6), and the like. Additional examples of alkynyl include heptynyl (C7), octynyl (C8), and the like.

The term “cycloalkyl” as used herein refers to a radical of a saturated or partially unsaturated cyclic hydrocarbon group having from 3 to 12 ring carbon atoms (“C3-12 cycloalkyl”) and zero heteroatoms in the ring system. In some embodiments, a cycloalkyl group has 3 to 10 ring carbon atoms (“C3-10 cycloalkyl”). In some embodiments, a cycloalkyl group has 3 to 8 ring carbon atoms (“C3-8 cycloalkyl”). In some embodiments, a cycloalkyl group has 3 to 6 ring carbon atoms (“C3-6 cycloalkyl”). In some embodiments, a cycloalkyl group has 3 to 6 ring carbon atoms (“C3-6 cycloalkyl”). In some embodiments, a cycloalkyl group has 5 to 10 ring carbon atoms (“C5-10 cycloalkyl”). Exemplary C3-6 cycloalkyl groups include, without limitation, cyclopropyl (C3), cyclopropenyl (C3), cyclobutyl (C₄), cyclobutenyl (C4), cyclopentyl (C5), cyclopentenyl (C5), bicyclo[1.1.1]pentyl (C5), cyclohexyl (C6), cyclohexenyl (C6), cyclohexadienyl (C6), and the like. Exemplary C3-8 cycloalkyl groups include, without limitation, the aforementioned C3-6 cycloalkyl groups as well as cycloheptyl (C7), cycloheptenyl (C7), cycloheptadienyl (C7), cycloheptatrienyl (C7), cyclooctyl (C8), cyclooctenyl (C8), bicyclo[2.2.1]heptanyl (C7), bicyclo[2.2.2]octanyl (C8), and the like. Exemplary C3-10 cycloalkyl groups include, without limitation, the aforementioned C3-8 cycloalkyl groups as well as cyclononyl (C9), cyclononenyl (C9), cyclodecyl (C10), cyclodecenyl (C10), octahydro-1H-indenyl (C9), decahydronaphthalenyl (C10), spiro[4.5]decanyl (C10), and the like. As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”). “Cycloalkyl” also includes ring systems wherein the cycloalkyl ring, as defined above, is fused with one or more aryl or heteroaryl groups wherein the point of attachment is on the cycloalkyl ring or the one or more aryl or heteroaryl groups, and in such instances, the number of carbons continue to designate the number of carbons in the cycloalkyl ring system.

The term “heterocyclyl” or “heterocycloalkyl” as used herein refers to a radical of a saturated or partially unsaturated 3 to 10-membered ring system having ring carbon atoms and 1 to 4 ring heteroatoms, wherein each heteroatom is independently selected from nitrogen, oxygen, sulfur, boron, phosphorus, and silicon (“3 to 10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”). Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring or the one or more aryl or heteroaryl groups, and in such instances, the number of ring members continue to designate the number of ring members in the heterocyclyl ring system.

In some embodiments, a heterocyclyl group is a 5 to 10 membered saturated or partially unsaturated ring system having ring carbon atoms and 1 to 4 ring heteroatoms, wherein each heteroatom is independently selected from nitrogen, oxygen, sulfur, boron, phosphorus, and silicon (“5 to 10 membered heterocyclyl”). In some embodiments, a heterocycloalkyl group is a 5 to 10 membered saturated or partially unsaturated ring system having ring carbon atoms and 1 to 4 ring heteroatoms, wherein each heteroatom is independently selected from nitrogen, oxygen, sulfur, boron, phosphorus, and silicon (“5 to 10 membered heterocyclyl”). In some embodiments, a heterocyclyl group is a 5 to 8 membered saturated or partially unsaturated ring system having ring carbon atoms and 1 to 4 ring heteroatoms, wherein each heteroatom is independently selected from nitrogen, oxygen, and sulfur (“5 to 8 membered heterocyclyl”). In some embodiments, a heterocyclyl group is a 5 to 6 membered saturated or partially unsaturated ring system having ring carbon atoms and 1 to 4 ring heteroatoms, wherein each heteroatom is independently selected from nitrogen, oxygen, and sulfur (“5 to 6 membered heterocyclyl”). In some embodiments, the 5 to 6 membered heterocyclyl has 1 to 3 ring heteroatoms selected from nitrogen, oxygen, and sulfur. In some embodiments, the 5 to 6 membered heterocyclyl has 1 to 2 ring heteroatoms selected from nitrogen, oxygen, and sulfur. In some embodiments, the 5 to 6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiiranyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C6 aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

The term “aryl” as used herein refers to a radical of a monocyclic or polycyclic (e.g., bicyclic or tricyclic) 4n+2 aromatic ring system (e.g., having 6, 10, or 14 π electrons shared in a cyclic array) having 6 to 14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, and trinaphthalene. Particularly aryl groups include phenyl, naphthyl, indenyl, and tetrahydronaphthyl.

The term “heteroaryl” as used herein refers to a radical of a 5 to 10 membered monocyclic or bicyclic 4n+2 aromatic ring system (e.g., having 6 or 10 π electrons shared in a cyclic array) having ring carbon atoms and 1 to 4 ring heteroatoms provided in the aromatic ring system, wherein each heteroatom is independently selected from nitrogen, oxygen and sulfur (“5 to 10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5 to 10 membered aromatic ring system having ring carbon atoms and 1 to 4 ring heteroatoms provided in the aromatic ring system, wherein each heteroatom is independently selected from nitrogen, oxygen, and sulfur (“5 to 10 membered heteroaryl”). In some embodiments, a heteroaryl group is a 5 to 8 membered aromatic ring system having ring carbon atoms and 1 to 4 ring heteroatoms provided in the aromatic ring system, wherein each heteroatom is independently selected from nitrogen, oxygen, and sulfur (“5 to 8 membered heteroaryl”). In some embodiments, a heteroaryl group is a monocyclic 5 to 6 membered aromatic ring system having ring carbon atoms and 1 to 4 ring heteroatoms provided in the aromatic ring system, wherein each heteroatom is independently selected from nitrogen, oxygen, and sulfur (“5 to 6 membered heteroaryl”). In some embodiments, the 5 to 6 membered heteroaryl has 1 to 3 ring heteroatoms selected from nitrogen, oxygen, and sulfur. In some embodiments, the 5 to 6 membered heteroaryl has 1 to 2 ring heteroatoms selected from nitrogen, oxygen, and sulfur. In some embodiments, the 5 to 6 membered heteroaryl has 1 ring heteroatom selected from nitrogen, oxygen, and sulfur. In some embodiments, a heteroaryl group is a monocyclic 5 membered aromatic ring system having ring carbon atoms and 1 to 4 ring heteroatoms provided in the aromatic ring system, wherein each heteroatom is independently selected from nitrogen, oxygen, and sulfur (“5-membered heteroaryl”). In some embodiments, a heteroaryl group is a monocyclic 6 membered aromatic ring system having ring carbon atoms and 1 to 4 ring heteroatoms provided in the aromatic ring system, wherein each heteroatom is independently selected from nitrogen, oxygen, and sulfur (“6-membered heteroaryl”).

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

The term “alkoxy” as used herein refers to the group —OR¹⁰⁰ where R¹⁰⁰ is alkyl, alkenyl, alkynyl, cycloalkyl, heterocyclyl, aryl, or heteroaryl. Exemplary alkoxy groups include methoxy, ethoxy, n-propoxy, isopropoxy, n-butoxy, tert-butoxy, sec-butoxy, n-pentoxy, n-hexoxy, and 1,2-dimethylbutoxy. Other exemplary alkoxy groups are lower alkoxy, i.e. with between 1 and 6 carbon atoms. In other examples, alkoxy groups have between 1 and 4 carbon atoms.

The term “thioalkoxy” as used herein refers to the group —SR¹⁰¹ where R¹⁰¹ is alkyl, alkenyl, alkynyl, cycloalkyl, heterocyclyl, aryl, or heteroaryl. Exemplary alkoxy groups include methoxy, ethoxy, n-propoxy, isopropoxy, n-butoxy, tert-butoxy, sec-butoxy, n-pentoxy, n-hexoxy, and 1,2-dimethylbutoxy. Other exemplary alkoxy groups are lower alkoxy, i.e. with between 1 and 6 carbon atoms. In other examples, alkoxy groups have between 1 and 4 carbon atoms.

The term “cyano” as used herein refers to the radical —CN.

The term “halogen” as used herein refers to F, Cl, Br, or I.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compounds of the present disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

A “subject” to which administration is contemplated includes, but is not limited to, humans (i.e., a male or female of any age group, e.g., a pediatric subject (e.g, infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “human,” “patient,” and “subject” are used interchangeably herein.

The terms “disease,” “disorder,” and “condition” are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition. In an alternative embodiment, the present disclosure contemplates administration of the compounds described herein as a prophylactic before a subject begins to suffer from the specified disease, disorder or condition.

In general, the “effective amount” of a compound as used herein refers to an amount sufficient to elicit the desired biological response. As will be appreciated by those of ordinary skill in this art, the effective amount of a compound of the present disclosure may vary depending on such factors as the desired biological endpoint, the pharmacokinetics of the compound, the disease being treated, the mode of administration, and the age, health, and condition of the subject.

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.” Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

Isomers, e.g., stereoisomers, can be isolated from mixtures by methods known to those skilled in the art, including chiral high pressure liquid chromatography (HPLC) and the formation of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The present disclosure additionally encompasses compounds described herein as individual isomers substantially free of other isomers, and alternatively, as mixtures of various isomers.

The compounds provided herein can be administered as the sole active agent, or they can be administered in combination with other active agents. In some embodiments, the present invention provides a combination of a compound of the present invention and another pharmacologically active agent. Administration in combination can proceed by any technique apparent to those of skill in the art including, for example, separate, sequential, concurrent, and alternating administration.

The present disclosure, in an alternative embodiment, also embraces isotopically labeled compounds which are identical to those recited herein, except that one or more atoms are replaced by an atom having an atomic mass or mass number different from the atomic mass or mass number usually found in nature. Examples of isotopes that can be incorporated into compounds described herein include isotopes of hydrogen, carbon, nitrogen, oxygen, phosphorus, sulfur, fluorine and chlorine, such as ²H, ³H, ¹³C, ¹⁴C, ¹⁵N, ¹⁸O, ¹⁷O, ³¹P, ³²P ³⁵S ¹⁸F, and ³⁶Cl, respectively. For example, a compound of the disclosure may have one or more H atom replaced with deuterium.

### EXAMPLES

The compounds provided herein can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization.

Abbreviations: eq: equivalents; ESI: electrospray ionization; h: hours; HPLC: high-performance liquid chromatography; MS: mass spectrometry; NMR: nuclear magnetic resonance; AcOH or HOAc: acetic acid; Boc: tert-butyloxycarbonyl; BOP: basic oxygen process; BrettPhos: dialkylbiaryl phosphine ligand; CDI: carbonyldiimidazole; DBU: 1,8-diazabicyclo [5.4.0]undec-7-ene; DCM: dichloromethane; DIEA: N,N-diisopropylethylamine; DMAP: 4-dimethylaminopyridine; DMF: dimethylformamide; DMSO: dimethyl sulfoxide; EDCI: ethylene dichloride; HOBt: hydroxybenzotriazole; LAH: lithium aluminium hydride; MeCN: acetonitrile; MeOH: methanol; Pd PEPPSI-IHetp Cl: dichloro[1,3-bis(2,6-di-4-heptylphenyl)imidazol-2-ylidene](3-chloropyridyl)palladium(II); Pd(PPh)4: palladium-tetrakis (triphenylphosphine); Py: pyridine; t-BuONa: sodium tert-butoxide; TBuONO: tert-butyl nitrite; TEA: triethylamine; TFA: trifluoroacetic acid; THF: tetrahydrofuran; triphosgene: bis(trichloromethyl) carbonate; ZnEt2: diethylzinc.

### Example 1. Synthesis of Compound 1

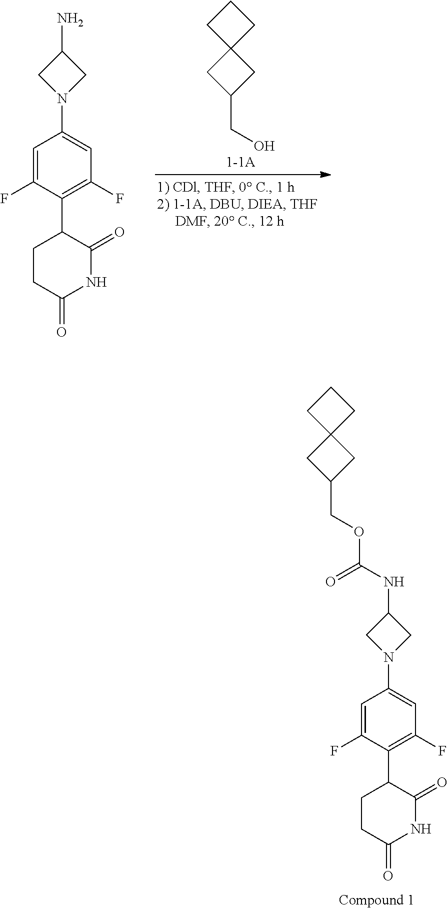 [SMILES: NC1CN(c2cc(F)c(C3CCC(=O)NC3=O)c(F)c2)C1 ; OCC1CC2(CCC2)C1 ; O=C1CCC(c2c(F)cc(N3CC(NC(=O)OCC4CC5(CCC5)C4)C3)cc2F)C(=O)N1]

### Step 1. Procedure for Compound 1—spiro[3.3]heptan-2-ylmethyl (1-(4-(2,6-dioxopiperidin-3-yl)-3,5-difluorophenyl)azetidin-3-yl)carbamate

To a solution of spiro[3.3]heptan-2-ylmethanol (5.98 mg, 47.4 umol, 1.00 eq) in tetrahydrofuran (0.200 mL) was added di(1H-imidazol-1-yl)methanone (15.4 mg, 94.8 umol, 2.00 eq) at 0° C. The mixture was stirred at 0° C. for 1 h. The resulting solution was added to a mixture of 3-(4-(3-aminoazetidin-1-yl)-2,6-difluorophenyl)piperidine-2,6-dione (20.0 mg, 47.4 umol, 70% purity, 1.00 eq), 2,3,4,6,7,8,9,10-octahydropyrimido[1,2-a]azepine (7.22 mg, 47.4 umol, 7.15 uL, 1.00 eq) and N,N-diisopropylethyl amine (6.13 mg, 47.4 umol, 8.26 uL, 1.00 eq) in tetrahydrofuran (0.200 mL) and dimethylformamide (0.200 mL). The reaction mixture was stirred at 20° C. for 12 h. The reaction mixture was concentrated under reduced pressure to give a residue. The residue was diluted with dimethylformamide (1.00 mL) and then filtered. The filtrate was purified by Prep-HPLC (column: Phenomenex luna C18 150*25 mm*10 um; mobile phase: [water(formic acid)-acetonitrile]; B %: 46%-76%, 9 min) and lyophilized to afford spiro[3.3]heptan-2-ylmethyl (1-(4-(2,6-dioxopiperidin-3-yl)-3,5-difluorophenyl)azetidin-3-yl)carbamate (3.93 mg, 8.69 umol, 18% yield, 99% purity) as an off-white solid.

### Example 2. Synthesis of Compound 2

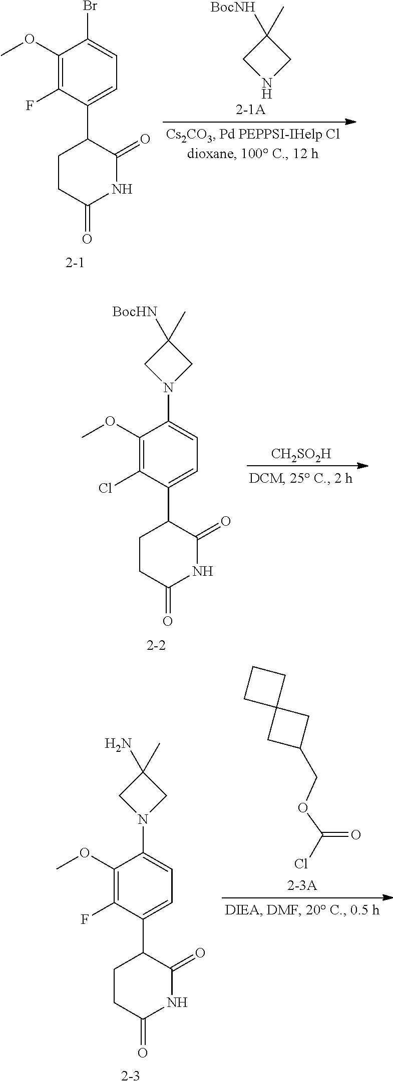 [SMILES: COc1c(Br)ccc(C2CCC(=O)NC2=O)c1F ; CC1(NC(=O)OC(C)(C)C)CNC1 ; COc1c(N2CC(C)(NC(=O)OC(C)(C)C)C2)ccc(C2CCC(=O)NC2=O)c1Cl ; "CH2SO2H" ; COc1c(N2CC(C)(N)C2)ccc(C2CCC(=O)NC2=O)c1F ; O=C(Cl)OCC1CC2(CCC2)C1]

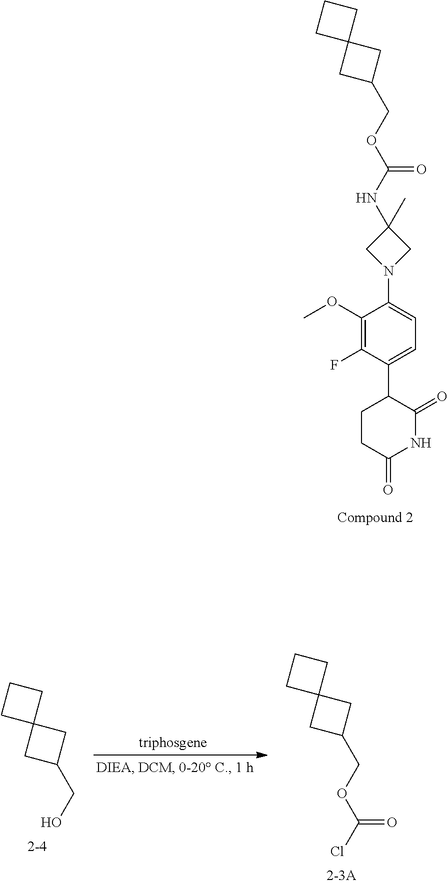 [SMILES: COc1c(N2CC(C)(NC(=O)OCC3CC4(CCC4)C3)C2)ccc(C2CCC(=O)NC2=O)c1F ; OCC1CC2(CCC2)C1 ; O=C(Cl)OCC1CC2(CCC2)C1]

### Step 1. Procedure for Compound 2—tert-butyl (1-(4-(2,6-dioxopiperidin-3-yl)-3-fluoro-2-methoxyphenyl)-3-methylazetidin-3-yl)carbamate

To a solution of 3-(4-bromo-2-fluoro-3-methoxyphenyl)piperidine-2,6-dione (100 mg, 316 umol, 1.00 eq), tert-butyl (3-methylazetidin-3-yl)carbamate (70.5 mg, 316 umol, 1.0 eq, hydrochloric acid) in dioxane (2.00 mL) were added cesium carbonate (309 mg, 949 umol, 3.00 eq) and 1,3-bis[2,6-bis(1-propylbutyl)phenyl]-4,5-dichloro-2H-imidazol-1-ium-2-ide;3-chloropyridine;dichloropalladium (30.8 mg, 31.6 umol, 0.100 eq). The mixture was stirred at 100° C. for 12 h under nitrogen atmosphere. The mixture was concentrated under reduced pressure to give a residue. The residue was purified by column chromatography (SiO₂, petroleum ether/ethyl acetate=1/0 to 5/1) to afford tert-butyl N-[1-[4-(2,6-dioxo-3-piperidyl)-3-fluoro-2-methoxy-phenyl]-3-methyl-azetidin-3-yl]carbamate (56.0 mg, 133 umol, 42% yield) as a white solid.

¹H NMR (400 MHz, DMSO-d₆) δ=10.80 (s, 1H), 7.42-7.28 (m, 1H), 6.79 (t, J=8.1 Hz, 1H), 6.21 (d, J=8.1 Hz, 1H), 3.93-3.83 (m, 3H), 3.73 (d, J=7.9 Hz, 2H), 3.67 (s, 3H), 2.76-2.67 (m, 1H), 2.58-2.53 (m, 1H), 2.17-2.06 (m, 1H), 1.98-1.89 (m, 1H), 1.51 (s, 3H), 1.38 (s, 9H).

### Step 2. Procedure for Compound 3—3-(4-(3-amino-3-methylazetidin-1-yl)-2-fluoro-3-methoxyphenyl)piperidine-2,6-dione

A solution of tert-butyl (1-(4-(2,6-dioxopiperidin-3-yl)-3-fluoro-2-methoxyphenyl)-3-methylazetidin-3-yl)carbamate (48.0 mg, 114 umol, 1.00 eq) in dichloromethane (2.00 mL) was added methanesulfonic acid (32.8 mg, 341 umol, 24.3 uL, 3.00 eq). The mixture was stirred at 25° C. for 2 h. The mixture was concentrated under reduced pressure to give 3-[4-(3-amino-3-methyl-azetidin-1-yl)-2-fluoro-3-methoxy-phenyl]piperidine-2,6-dione (40.0 mg, crude) as yellow oil. MS (ESI) m/z. 322.1 [M+H]⁺

### Step 3. Procedure for Compound 3A—spiro[3.3]heptan-2-ylmethyl carbonochloridate

A solution of spiro[3.3]heptan-2-ylmethanol (30.0 mg, 238 umol, 1.00 eq) in dichloromethane (1.00 mL) was added N,N-diisopropylethylamine (61.5 mg, 475 umol, 82.8 uL, 2.00 eq) and triphosgene (106 mg, 357 umol, 1.5 eq) at 0° C. The mixture was stirred at 20° C. for 1 h. The mixture was concentrated under reduced pressure to give spiro[3.3]heptan-2-ylmethyl carbonochloridate (45 mg, crude) as a white solid.

### Step 4. Procedure for spiro[3.3]heptan-2-ylmethyl (1-(4-(2,6-dioxopiperidin-3-yl)-3-fluoro-2-methoxyphenyl)-3-methylazetidin-3-yl)carbamate

To a solution of 3-[4-(3-amino-3-methyl-azetidin-1-yl)-2-fluoro-3-methoxy-phenyl]piperidine-2,6-dione (40.0 mg, 124 umol, 1.00 eq) in dimethyl formamide (1.00 mL) were added N,N-diisopropylethylamine (16.1 mg, 124 umol, 21.7 uL, 1.00 eq) and spiro[3.3]heptan-2-ylmethyl carbonochloridate (45.0 mg, 238.54 umol, 1.92 eq). The reaction mixture was stirred at 20° C. for 0.5 h. The reaction mixture was concentrated under reduced pressure to give a residue. The residue was purified by prep-HPLC (C18, 120 g; condition:water/acetonitrile=100:0 to 60:40, 0.1% formic acid) and lyophilized to afford spiro[3.3]heptan-2-ylmethyl (1-(4-(2,6-dioxopiperidin-3-yl)-3-fluoro-2-methoxyphenyl)-3-methylazetidin-3-yl)carbamate (10.44 mg, 21.8 umol, 17% yield, 99% purity) as a white solid.

¹H NMR (400 MHz, DMSO-d₆) δ=10.80 (br s, 1H), 7.76-7.50 (m, 1H), 6.79 (t, J=8.1 Hz, 1H), 6.20 (d, J=8.4 Hz, 1H), 3.88 (br d, J=6.8 Hz, 5H), 3.75 (d, J=7.8 Hz, 2H), 3.67 (s, 3H), 2.75-2.66 (m, 1H), 2.54-2.51 (m, 1H), 2.34 (td, J=7.5, 14.8 Hz, 1H), 2.20-2.10 (m, 1H), 2.04-1.93 (m, 5H), 1.89-1.84 (m, 2H), 1.79-1.67 (m, 4H), 1.51 (s, 3H). MS (ESI) m/z. 474.3 [M+H]⁺

### Example 3. Synthesis of Compound 3

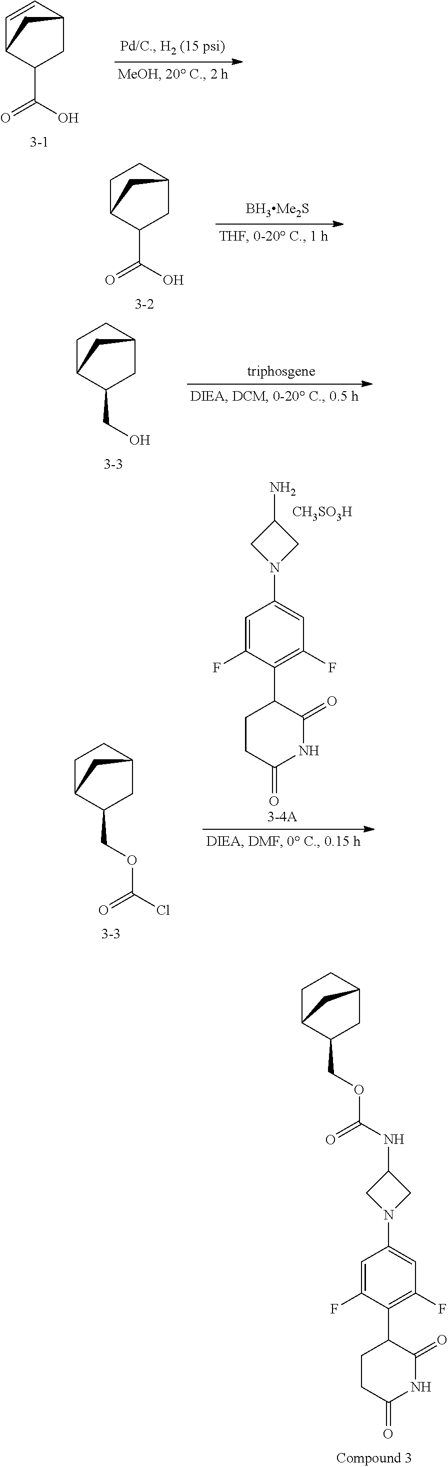

### Step 1. Procedure for Preparation of Compound 2—(1R,2R,4S)-bicyclo[2.2.1]heptane-2-carboxylic acid

To a solution of (1S,2R,4S)-bicyclo[2.2.1]hept-5-ene-2-carboxylic acid (50.0 mg, 362 umol, 1.00 eq) in methanol (1.00 mL) was added palladium on carbon (50.0 mg, 10% purity) under nitrogen atmosphere. The reaction mixture was stirred at 20° C. for 1 h under hydrogen atmosphere (15 psi). The reaction mixture was filtered and the filtrate was concentrated under reduced pressure to afford (1R,2R,4S)-bicyclo[2.2.1]heptane-2-carboxylic acid (50.0 mg, 357 umol, 99% yield) as colorless oil.

¹H NMR (400 MHz, CHCl₃-d) δ=2.58 (br s, 1H), 2.39 (br dd, J=5.4, 8.6 Hz, 1H), 2.33 (br s, 1H), 1.92-1.81 (m, 1H), 1.66-1.54 (m, 2H), 1.54-1.47 (m, 2H), 1.32-1.26 (m, 1H), 1.21 (br d, J=8.8 Hz, 2H).

### Step 2. Procedure for Preparation of Compound 3—(1R,2R,4S)-bicyclo[2.2.1]heptan-2-ylmethanol

To a solution of (1R,2R,4S)-bicyclo[2.2.1]heptane-2-carboxylic acid (50.0 mg, 357 umol, 1.00 eq) in tetrahydrofuran (1.00 mL) was added borane dimethyl sulfide complex (10.0 M, 71.3 uL, 2.00 eq) at 0° C. The reaction mixture was stirred at 20° C. for 1 h. The reaction mixture was concentrated under reduced pressure to afford (1R,2R,4S)-bicyclo[2.2.1]heptan-2-ylmethanol (45.0 mg, 357 umol, 99% yield) as colorless oil.

¹H NMR (400 MHz, CHCl₃-d) δ=3.42-3.29 (m, 2H), 2.22 (br s, 1H), 2.17 (br s, 1H), 1.69-1.65 (m, 1H), 1.55-1.49 (m, 2H), 1.40-1.35 (m, 1H), 1.28-1.10 (m, 4H), 1.00 (ddd, J=4.6, 7.2, 12.0 Hz, 1H).

### Step 3. Procedure for Preparation of Compound 4—(1R,2R,4S)-bicyclo[2.2.1]heptan-2-ylmethyl carbonochloridate

To a solution of (1R,2R,4S)-bicyclo[2.2.1]heptan-2-ylmethanol (45.0 mg, 357 umol, 1.00 eq) in dichloromethane (1.00 mL) were added bis(trichloromethyl) carbonate (106 mg, 357 umol, 1.00 eq) and N,N-diisopropylethylamine (184 mg, 1.43 mmol, 248 uL, 4.00 eq) at 0° C. Then the reaction was stirred at 20° C. for 0.5 h. The reaction mixture was concentrated under reduced pressure to afford (1R,2R,4S)-bicyclo[2.2.1]heptan-2-ylmethyl carbonochloridate (65 mg, crude) as a yellow solid.

### Step 4. Procedure for Preparation of (1R,2R,4S)-bicyclo[2.2.1]heptan-2-ylmethyl (1-(4-(2,6-dioxopiperidin-3-yl)-3,5-difluorophenyl)azetidin-3-yl)carbamate

To the solution of 3-(4-(3-aminoazetidin-1-yl)-2,6-difluorophenyl)piperidine-2,6-dione (111 mg, 284 umol, 1.00 eq, mesylate) in dimethylformamide (1.00 mL) were added N,N-diisopropylethylamine (134 mg, 1.03 mmol, 180 uL, 3.00 eq) and (1R,2R,4S)-bicyclo[2.2.1]heptan-2-ylmethyl carbonochloridate (65.0 mg, crude). Then the reaction was stirred at 0° C. for 0.15 h. The reaction mixture was diluted with N,N-dimethylformamide (1.00 mL) and filtered. The filtrate was purified by reverse phase chromatography (C18, 40 g; condition: water/acetonitrile=100:0 to 0:100, 0.1% formic) and lyophilized to afford (1R,2R,4S)-bicyclo[2.2.1]heptan-2-ylmethyl (1-(4-(2,6-dioxopiperidin-3-yl)-3,5-difluorophenyl)azetidin-3-yl)carbamate (50.00 mg, 110.62 umol, 32.11% yield, 99% purity) as a white solid.

¹H NMR (400 MHz, DMSO-d₆) δ=10.85 (s, 1H), 7.83 (br d, J=7.1 Hz, 1H), 6.14 (d, J=11.1 Hz, 2H), 4.50-4.30 (m, 1H), 4.08 (br t, J=7.7 Hz, 2H), 4.03 (br dd, J=5.1, 12.8 Hz, 1H), 3.77-3.66 (m, 2H), 3.63 (br t, J=6.8 Hz, 2H), 2.85-2.70 (m, 1H), 2.48 (br s, 1H), 2.18 (br s, 1H), 2.12-2.02 (m, 2H), 1.99-1.89 (m, 1H), 1.76-1.64 (m, 1H), 1.53-1.40 (m, 2H), 1.36-1.25 (m, 2H), 1.19-0.96 (m, 4H). MS (ESI) m/z 448.2 [M+H]⁺

### Example 4. Synthesis of Compound 4

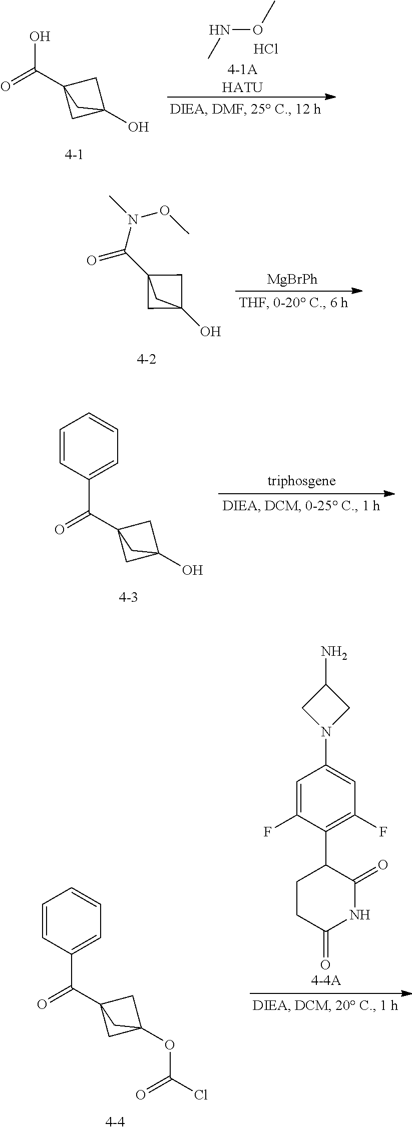

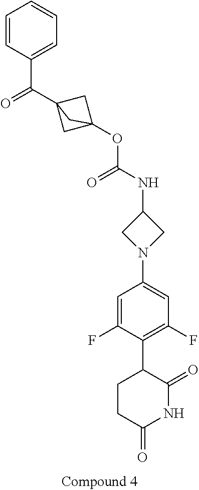 [SMILES: O=C1CCC(c2c(F)cc(N3CC(NC(=O)OC45CC(C(=O)c6ccccc6)(C4)C5)C3)cc2F)C(=O)N1]

### Step 1. Procedure for Preparation of Compound 2—3-hydroxy-N-methoxy-N-methylbicyclo[1.1.1]pentane-1-carboxamide

To a solution of 3-hydroxybicyclo[1.1.1]pentane-1-carboxylic acid (100 mg, 780 umol, 1.00 eq) in dimethyl formamide (1.00 mL) was added o-(7-azabenzotriazol-1-yl)-N,N,N′,N′-tetramethyluronium hexafluorophosphate (356.12 mg, 936.58 umol, 1.2 eq), N,N-diisopropylethylamine (403 mg, 3.12 mmol, 544 uL, 4.00 eq) at 25° C. to afford mixture A. To a solution of N, O-dimethylhydroxylamine (91.4 mg, 937 umol, 1.20 eq, hydrochloride) in dimethyl formamide (1.00 mL) was added N,N-diisopropylethylamine (202 mg, 1.56 mmol, 272 uL, 2.00 eq) to give mixture B. The mixture B was added into the mixture A at 25° C. The mixture was stirred at 25° C. for 12 h. The reaction mixture was concentrated under reduced pressure to give a residue. The residue was purified by column chromatography (SiO₂, petroleum ether/ethyl acetate=3/1 to 1/1) to afford 3-hydroxy-N-methoxy-N-methylbicyclo[1.1.1]pentane-1-carboxamide (50.0 mg, 292 umol, 37.42% yield) as colorless oil.

¹H NMR (400 MHz, CDCl₃-d) δ=3.67 (s, 3H), 3.20 (s, 3H), 2.30-2.23 (m, 6H).

### Step 2. Procedure for Preparation of Compound 3—(3-hydroxybicyclo[1.1.1]pentan-1-yl)(phenyl)methanone

A solution of 3-hydroxy-N-methoxy-N-methylbicyclo[1.1.1]pentane-1-carboxamide (210 mg, 1.23 mmol, 1.00 eq) in tetrahydrofuran (10.0 mL) was degassed and purged with nitrogen atmosphere. The resulting clear solution was cooled to 0° C. And then a solution of phenylmagnesium bromide (3 M, 1.23 mL, 3.00 eq) was added dropwise to the mixture via syringe. After 20 min, the mixture was allowed to warm to 25° C. for 6 h. The reaction mixture was quenched by addition ammonium chloride (10.0 mL) at 0° C. and extracted with ethyl acetate 20 mL (10 mL*2). The combined organic layers were dried over sodium sulfate, filtered and concentrated under reduced pressure to give a residue. The residue was purified by column chromatography (SiO₂, petroleum ether/ethyl acetate=3/1) to afford (3-hydroxybicyclo[1.1.1]pentan-1-yl)(phenyl)methanone (160 mg, 850 umol, 69.0% yield) as light yellow oil.

¹H NMR (400 MHz, CDCl₃-d) δ=7.88 (d, J=7.6 Hz, 2H), 7.51-7.46 (m, 1H), 7.42-7.35 (m, 2H), 2.38 (s, 6H)

### Step 3. Procedure for Preparation of Compound 4—3-benzoylbicyclo[1.1.1]pentan-1-yl carbonochloridate

To a solution of (3-hydroxybicyclo[1.1.1]pentan-1-yl)(phenyl)methanone (60.0 mg, 319 umol, 1.00 eq) in dichloromethane (2.00 mL) was added triphosgene (151 mg, 510 umol, 1.60 eq) and N,N-diisopropylethylamine (82.4 mg, 638 umol, 111 uL, 2.00 eq) at 0° C. The mixture was stirred at 25° C. for 1 h. The reaction mixture was filtered and concentrated under reduced pressure to afford 3-benzoylbicyclo[1.1.1]pentan-1-yl carbonochloridate (70.0 mg, 279 umol, 88% yield) as a yellow solid.

### Step 4. Procedure for Preparation of Compound 4—1-(methyl(p-tolyl)carbamoyl)azetidin-3-yl (1-(4-(2,6-dioxopiperidin-3-yl)-3,5-difluorophenyl)azetidin-3-yl)carbamate

To a solution of 3-(4-(3-aminoazetidin-1-yl)-2,6-difluorophenyl)piperidine-2,6-dione (100 mg, 256 umol, 1.00 eq, methanesulfonic acid) in dichloromethane (3.00 mL) was added N,N-diisopropylethylamine (33.0 mg, 256 umol, 44.5 uL, 1.00 eq) and 3-benzoylbicyclo[1.1.1]pentan-1-yl carbonochloridate (70 mg, 279 umol, 1.09 eq). The mixture was stirred at 20° C. for 1 h. The reaction mixture was concentrated under reduced pressure to give a residue. The residue was purified by purified by reverse phase chromatography (C18, 40 g; condition: water/acetonitrile=100:0 to 0:100, 0.1% formic) and lyophilized to afford 1-(methyl(p-tolyl)carbamoyl)azetidin-3-yl (1-(4-(2,6-dioxopiperidin-3-yl)-3,5-difluorophenyl)azetidin-3-yl)carbamate (11.29 mg, 21.72 umol, 8.50% yield, 98% purity) as a white solid.

¹H NMR (400 MHz, DMSO-d₆) δ=10.86 (s, 1H), 8.03 (d, J=7.4 Hz, 1H), 7.96 (d, J=7.4 Hz, 2H), 7.69-7.62 (m, 1H), 7.58-7.51 (m, 2H), 6.16 (d, J=11.0 Hz, 2H), 4.50-4.36 (m, 1H), 4.11 (t, J=7.7 Hz, 2H), 4.04 (br dd, J=5.2, 12.8 Hz, 1H), 3.70-3.60 (m, 2H), 2.84-2.73 (m, 1H), 2.59 (s, 6H), 2.55-2.54 (m, 1H), 2.15-2.03 (m, 1H), 2.01-1.90 (m, 1H). MS (ESI) m/z 510.3 [M+H]⁺

### Example 5. Synthesis of Compound 5

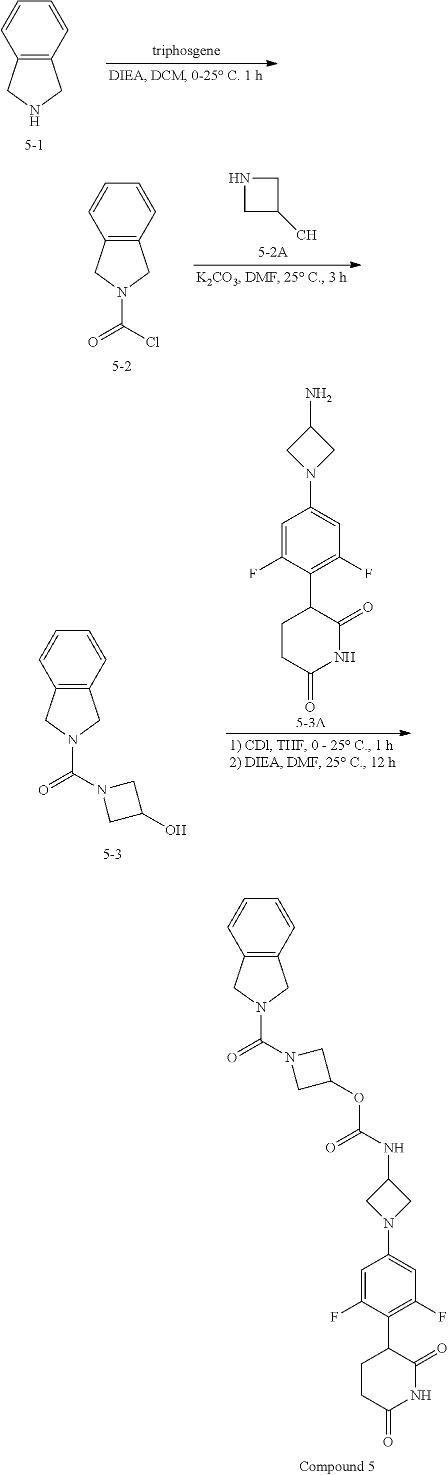

### Step 1. Procedure for Preparation of Compound 2—isoindoline-2-carbonyl chloride

To a solution of isoindoline (950 mg, 7.97 mmol, 904 uL, 1.00 eq) in dichloromethane (10.0 mL) was added triphosgene (2.84 g, 9.57 mmol, 1.20 eq) and N,N-diisopropylethylamine (1.55 g, 11.9 mmol, 2.08 mL, 1.50 eq) at 0° C. The mixture was stirred at 25° C. for 1 h The reaction mixture was concentrated under reduce pressure to afford isoindoline-2-carbonyl chloride (1.50 g, crude) as a yellow solid.

### Step 2. Procedure for Preparation of Compound 3—(3-hydroxyazetidin-1-yl)(isoindolin-2-yl)methanone

To a solution of isoindoline-2-carbonyl chloride (1.45 g, 7.98 mmol, 1.00 eq) in dimethylformamide (10.0 mL) was added potassium carbonate (8.83 g, 64.0 mmol, 8.00 eq) and azetidin-3-ol (1.75 g, 15.97 mmol, 2.00 eq, hydrochloride). The mixture was stirred at 25° C. for 3 h. The reaction mixture was concentrated under reduced pressure to give a residue. The residue was purified by column chromatography (SiO₂, petroleum ether/ethyl acetate=1/3 to 0/1) to afford (3-hydroxyazetidin-1-yl)(isoindolin-2-yl)methanone (700 mg, 3.21 mmol, 40% yield) as an off-white solid.

¹H NMR (400 MHz, DMSO-d₆) δ=7.33-7.26 (m, 4H), 4.61 (s, 4H), 4.45-4.39 (m, 1H), 4.15 (d, J=7.9 Hz, 2H), 3.74 (dd, J=4.9, 8.8 Hz, 2H), 3.60 (dt, J=3.8, 6.4 Hz, 1H)

### Step 3. Procedure for Preparation of 1-(isoindoline-2-carbonyl)azetidin-3-yl (1-(4-(2,6-dioxopiperidin-3-yl)-3,5-difluorophenyl)azetidin-3-yl)carbamate

To a solution of (3-hydroxyazetidin-1-yl)(isoindolin-2-yl)methanone (100 mg, 275 umol, 60% purity, 1.00 eq) in tetrahydrofuran (2.00 mL) was added 1,1′-carbonyldiimidazole (66.9 mg, 412 umol, 1.50 eq). The mixture was stirred at 25° C. for 1 h to give a resulting solution. A solution of 3-(4-(3-aminoazetidin-1-yl)-2,6-difluorophenyl)piperidine-2,6-dione (107 mg, 273 umol, 1.00 eq, mesylate) and N,N-diisopropylethylamine (53.0 mg, 410 umol, 71.4 uL, 1.50 eq) in dimethylformamide (1.00 mL) was added into the resulting solution. The mixture was stirred at 25° C. for 12 h. The mixture was purified by Prep-HPLC (Phenomenex luna C18 150*25 mm*10 um; mobile phase: [water(formic acid)-acetonitrile]; B %: 31%-61%, 9 min) and lyophilized to afford 1-(isoindoline-2-carbonyl)azetidin-3-yl (1-(4-(2,6-dioxopiperidin-3-yl)-3,5-difluorophenyl)azetidin-3-yl)carbamate (17.53 mg, 32.17 umol, 11% yield, 99% purity) as a white solid.

¹H NMR (400 MHz, DMSO-d₆) δ=10.87-10.81 (m, 1H), 8.13 (br d, J=7.1 Hz, 1H), 7.34-7.23 (m, 4H), 6.16 (br d, J=11.1 Hz, 2H), 5.09-5.00 (m, 1H), 4.62 (s, 4H), 4.49-4.39 (m, 1H), 4.29 (dd, J=6.9, 9.3 Hz, 2H), 4.11 (br t, J=7.6 Hz, 2H), 4.03 (br dd, J=4.9, 12.4 Hz, 1H), 3.89 (br dd, J=3.8, 9.3 Hz, 2H), 3.66 (br t, J=6.6 Hz, 2H), 2.83-2.71 (m, 1H), 2.47-2.41 (m, 1H), 2.13-2.03 (m, 1H), 1.98-1.89 (m, 1H). MS (ESI) m/z 539.9 [M+H]⁺

### Example 6. Synthesis of Compound 6

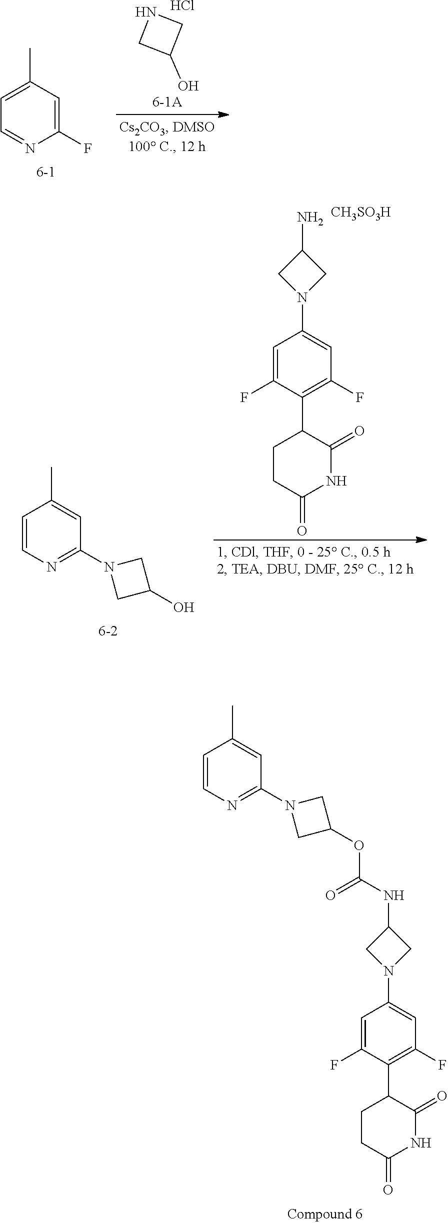

### Step 1. Procedure for Preparation of Compound 2—1-(4-methylpyridin-2-yl)azetidin-3-ol

To a solution of 2-fluoro-4-methylpyridine (500 mg, 4.50 mmol, 1.00 eq) in dimethylsulfoxide (5.00 mL) was added azetidin-3-ol (592 mg, 5.40 mmol, 1.20 eq, hydrochloride) and cesium carbonate (2.93 g, 9.00 mmol, 2.00 eq). The mixture was stirred at 100° C. for 12 h. The reaction mixture was concentrated under reduced pressure to give a residue. The residue was purified by reverse phase chromatography (C18, 40 g; condition: water/acetonitrile=100:0 to 0:100, 0.1% formic acid) and lyophilized to afford 1-(4-methylpyridin-2-yl)azetidin-3-ol (730 mg, 4.45 mmol, 98% yield) as colorless oil.

¹H NMR (400 MHz, DMSO-d₆) δ=8.14 (s, 1H), 7.89 (d, J=5.1 Hz, 1H), 6.45 (d, J=4.9 Hz, 1H), 6.19 (s, 1H), 4.58-4.51 (m, 1H), 4.10 (s, 2H), 3.63-3.60 (m, 2H), 2.19 (s, 3H). MS (ESI) m/z 165.4 [M+H]⁺

### Step 2. Procedure for Preparation of 1-(4-methylpyridin-2-yl)azetidin-3-yl (1-(4-(2,6-dioxopiperidin-3-yl)-3,5-difluorophenyl)azetidin-3-yl)carbamate

To a solution of 1-(4-methylpyridin-2-yl)azetidin-3-ol (300 mg, 1.83 mmol, 1.00 eq) in tetrahydrofuran (4.00 mL) was added 1,1′-carbonyldiimidazole (311 mg, 1.92 mmol, 1.05 eq). The mixture was stirred at 25° C. for 0.5 h. The resulting solution was added to a mixture of 3-(4-(3-aminoazetidin-1-yl)-2,6-difluorophenyl)piperidine-2,6-dione (474 mg, 1.21 mmol, 1.00 eq, methanesulfonic acid), triethylamine (123 mg, 1.21 mmol, 168 uL, 1.00 eq) and 1,8-diazabicyclo[5.4.0]undec-7-ene (184 mg, 1.21 mmol, 182 uL, 1.00 eq) in dimethylformamide (2.00 mL). The mixture was stirred at 25° C. for 12 h. The reaction mixture was concentrated under reduced pressure to give a residue. The residue was purified by column chromatography (SiO₂, petroleum ether/ethyl acetate=5/1 to 0/1) to afford a crude product. The crude product was triturated with water (10.0 ml) and filtered. The filter cake was washed with petroleum ether (3×5.00 ml) and lyophilized to afford 1-(4-methylpyridin-2-yl)azetidin-3-yl (1-(4-(2,6-dioxopiperidin-3-yl)-3,5-difluorophenyl)azetidin-3-yl)carbamate (19.18 mg, 39.1 umol, 3.22% yield, 99.0% purity) as a white solid.

¹H NMR (400 MHz, DMSO-d₆) δ=10.87 (s, 1H), 8.23 (br d, J=7.3 Hz, 1H), 7.90 (d, J=6.4 Hz, 1H), 6.87-6.65 (m, 2H), 6.17 (br d, J=11.1 Hz, 2H), 5.29-5.15 (m, 1H), 4.59-4.50 (m, 2H), 4.49-4.40 (m, 1H), 4.20-4.14 (m, 2H), 4.14-4.08 (m, 2H), 4.04 (br dd, J=4.9, 12.5 Hz, 1H), 3.67 (br t, J=6.8 Hz, 2H), 2.85-2.73 (m, 1H), 2.50-2.47 (m, 1H), 2.36 (s, 3H), 2.15-2.02 (m, 1H), 2.00-1.90 (m, 1H). MS (ESI) m/z 486.4 [M+H]⁺

### Example 7. Synthesis of Compound 7

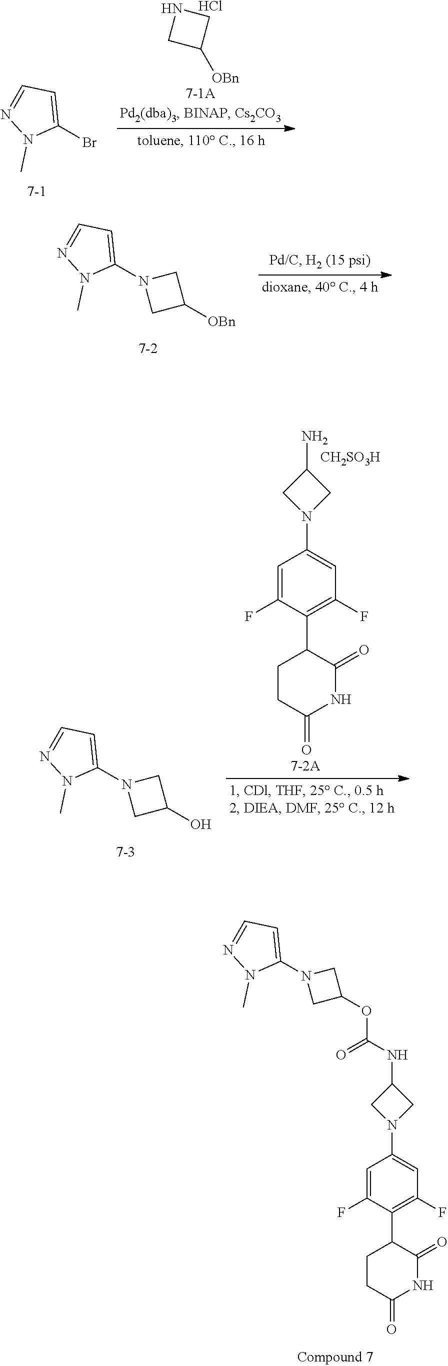

### Step 1. Procedure for Preparation of Compound 2—5-(3-(benzyloxy)azetidin-1-yl)-1-methyl-1H-pyrazole

To a solution of 5-bromo-1-methyl-1H-pyrazole (100 mg, 621 umol, 1.00 eq) in toluene (4.00 mL) was added 3-(benzyloxy)azetidine hydrochloride (62.0 mg, 311 umol, 0.500 eq, hydrochloric acid), cesium carbonate (809 mg, 2.48 mmol, 4.00 eq) and tris(dibenzylideneacetone)dipalladium(0) (56.9 mg, 62.1 umol, 0.100 eq) and 2,2′-bis-(diphenylphosphino)-1,1′-binaphthyl (77.4 mg, 124 umol, 0.200 eq). The mixture was stirred at 110° C. for 16 h. The reaction mixture was quenched by addition water (10.0 mL) at 20° C. and extracted with ethyl acetate (20.0 mL). The combined organic layers were washed with water (10.0 mL) and dried over sodium sulfate, filtered and concentrated under reduced pressure to give a residue. The residue was purified by reverse phase chromatography (C18, 40 g; condition: water/acetonitrile=100:0 to 0:100, 0.1% formic acid) to afford 5-(3-(benzyloxy)azetidin-1-yl)-1-methyl-1H-pyrazole (90.0 mg, 370 umol, 60% yield) as a yellow oil.

¹H NMR (400 MHz, CDCl₃) δ=7.41-7.30 (m, 5H), 7.28 (d, J=1.9 Hz, 1H), 5.54 (d, J=2.0 Hz, 1H), 4.51 (s, 2H), 4.49-4.41 (m, 1H), 4.08-3.99 (m, 2H), 3.76-3.69 (m, 2H), 3.63 (s, 3H)

### Step 2. Procedure for Preparation of Compound 3—1-(1-methyl-1H-pyrazol-5-yl)azetidin-3-ol

To a solution of 5-(3-(benzyloxy)azetidin-1-yl)-1-methyl-1H-pyrazole (50.0 mg, 206 umol, 1.00 eq) in dioxane (1.00 mL) was added palladium on activated carbon (50.0 mg, 60% purity, 1.00 eq). The mixture was stirred at 40° C. for 4 h under hydrogen atmosphere. The reaction mixture was concentrated under reduce pressure to afford 1-(1-methyl-1H-pyrazol-5-yl)azetidin-3-ol (100 mg, crude) as yellow oil, which was used for next step without further purification.

### Step 3. Procedure for Preparation of 1-(1-methyl-1H-pyrazol-5-yl)azetidin-3-yl (1-(4-(2,6-dioxopiperidin-3-yl)-3,5-difluorophenyl)azetidin-3-yl)carbamate

To a solution of 1-(1-methyl-1H-pyrazol-5-yl)azetidin-3-ol (30 mg, 196 umol, 1.00 eq) in tetrahydrofuran (0.50 mL) was added di(1H-imidazol-1-yl)methanone (38.1 mg, 235 umol, 1.20 eq). The mixture was stirred at 25° C. for 0.5 h. The resulting solution was added to a mixture of 3-(4-(3-aminoazetidin-1-yl)-2,6-difluorophenyl)piperidine-2,6-dione (76.7 mg, 196 umol, 1.00 eq, methanesulfonic acid) and N,N-diisopropylethylamine (75.9 mg, 588 umol, 102 μL, 3.00 eq) in dimethylformamide (1.00 mL). The reaction mixture was stirred at 25° C. for 12 h. The reaction mixture was concentrated under reduced pressure to give a residue. The residue was purified by reverse phase chromatography (C18, 40 g, condition: water/acetonitrile=100:0 to 0:1, 0.1% formic acid) and lyophilized to afford 1-(1-methyl-1H-pyrazol-5-yl)azetidin-3-yl (1-(4-(2,6-dioxopiperidin-3-yl)-3,5-difluorophenyl)azetidin-3-yl)carbamate (10.29 mg, 21.69 umol, 11.07% yield) as a white solid.

¹H NMR (400 MHz, DMSO-d₆) δ=10.86 (s, 1H), 8.13 (br d, J=7.1 Hz, 1H), 7.14 (d, J=1.8 Hz, 1H), 6.15 (d, J=11.1 Hz, 2H), 5.59 (d, J=1.3 Hz, 1H), 5.19-5.07 (m, 1H), 4.43 (br d, J=7.3 Hz, 1H), 4.18-4.07 (m, 4H), 4.03 (br dd, J=5.1, 12.4 Hz, 1H), 3.75-3.68 (m, 2H), 3.65 (br t, J=6.9 Hz, 2H), 3.52 (s, 3H), 2.83-2.72 (m, 1H), 2.62-2.57 (m, 1H), 2.13-2.01 (m, 1H), 1.99-1.88 (m, 1H). MS (ESI) m/z. 475.2 [M+H]⁺

### Example 8. Synthesis of Compound 8

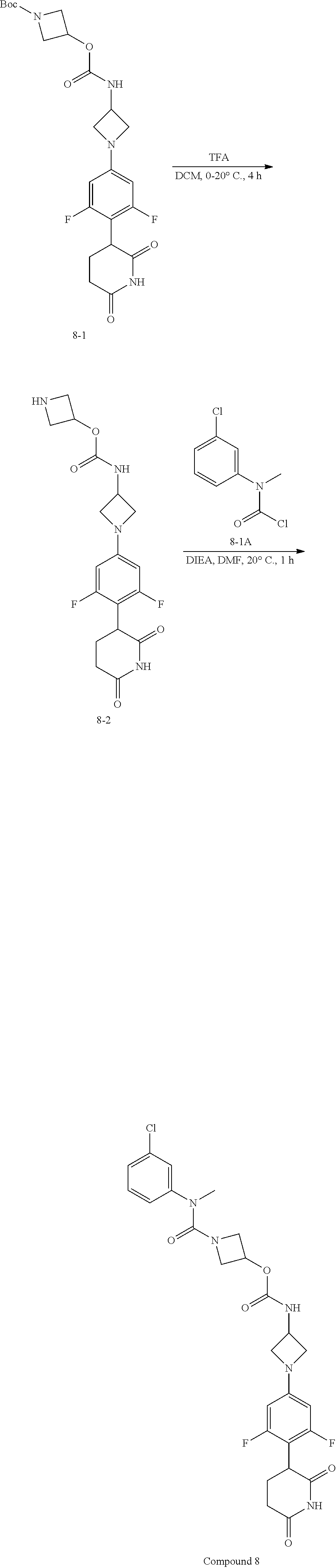 [SMILES: CC(C)(C)OC(=O)N1CC(OC(=O)NC2CN(c3cc(F)c(C4CCC(=O)NC4=O)c(F)c3)C2)C1 ; O=C1CCC(c2c(F)cc(N3CC(NC(=O)OC4CNC4)C3)cc2F)C(=O)N1 ; CN(C(=O)Cl)c1cccc(Cl)c1 ; CN(C(=O)N1CC(OC(=O)NC2CN(c3cc(F)c(C4CCC(=O)NC4=O)c(F)c3)C2)C1)c1cccc(Cl)c1]

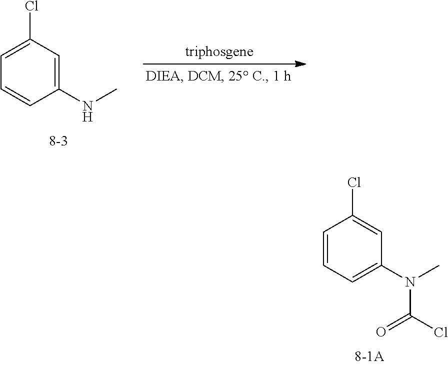

### Step 1. Procedure for compound 1A—(3-chlorophenyl)(methyl)carbamic chloride

To a solution of 3-chloro-N-methylaniline (45.0 mg, 318 umol, 38.8 uL, 1.00 eq) in dichloromethane (5.00 mL) was added N,N-diisopropylethylamine (82.2 mg, 636 umol, 111 uL, 2.00 eq) and bis(trichloromethyl) carbonate (151 mg, 508 umol, 1.60 eq) at 25° C. The mixture was stirred at 25° C. for 1 h. The reaction mixture was filtered and concentrated under reduced pressure to afford (3-chlorophenyl)(methyl)carbamic chloride (64.0 mg, 314 umol, 99% yield) as a brown solid.

### Step 2. Procedure for Compound 2—azetidin-3-yl (1-(4-(2,6-dioxopiperidin-3-yl)-3,5-difluorophenyl)azetidin-3-yl)carbamate

To a solution of tert-butyl 3-(((1-(4-(2,6-dioxopiperidin-3-yl)-3,5-difluorophenyl)azetidin-3-yl)carbamoyl)oxy)azetidine-1-carboxylate (100 mg, 202 umol, 1.00 eq) in dichloromethane (5.00 mL) was added trifluoroacetic acid (1.54 g, 13.5 mmol, 1.00 mL, 66.8 eq) at 0° C. The mixture was stirred at 20° C. for 4 h. The reaction mixture was concentrated under reduced pressure to afford azetidin-3-yl (1-(4-(2,6-dioxopiperidin-3-yl)-3,5-difluorophenyl)azetidin-3-yl)carbamate (102 mg, crude, trifluoroacetic acid) as colorless oil.

### Step 3. Procedure for 1-((3-chlorophenyl)(methyl)carbamoyl)azetidin-3-yl (1-(4-(2,6-dioxopiperidin-3-yl)-3,5-difluorophenyl)azetidin-3-yl)carbamate

To a solution of azetidin-3-yl (1-(4-(2,6-dioxopiperidin-3-yl)-3,5-difluorophenyl)azetidin-3-yl)carbamate (102 mg, 201 umol, 1.00 eq, trifluoroacetic acid) in dichloromethane (3.00 mL) was added N,N-diisopropylethylamine (77.8 mg, 602 umol, 105 uL, 3.00 eq) and (3-chlorophenyl)(methyl)carbamic chloride (49.1 mg, 241 umol, 1.20 eq). The mixture was stirred at 20° C. for 1 h. The reaction mixture was concentrated under reduced pressure to give a residue. The residue was purified by prep-HPLC (column: Phenomenex luna C18 150*25 mm*10 um; mobile phase: [water(formid acid)-acetonitrile]; B %: 32%-62%, 10 min) and lyophilized to afford 1-((3-chlorophenyl)(methyl)carbamoyl)azetidin-3-yl (1-(4-(2,6-dioxopiperidin-3-yl)-3,5-difluorophenyl)azetidin-3-yl)carbamate (27.53 mg, 51.6 umol, 25.7% yield) as an off-white solid.

¹H NMR (400 MHz, DMSO-d₆) δ=10.85 (s, 1H), 8.06 (br d, J=7.3 Hz, 1H), 7.45-7.36 (m, 2H), 7.26 (br dd, J=8.3, 15.8 Hz, 2H), 6.13 (br d, J=11.1 Hz, 2H), 4.89-4.82 (m, 1H), 4.43-4.31 (m, 1H), 4.11-4.01 (m, 3H), 3.87-3.79 (m, 2H), 3.60 (br t, J=6.7 Hz, 2H), 3.43 (br dd, J=3.3, 9.3 Hz, 2H), 3.14 (s, 3H), 2.82-2.72 (m, 1H), 2.56-2.53 (m, 1H), 2.10-2.01 (m, 1H), 1.98-1.89 (m, 1H). MS (ESI) m/z 562.4 [M+H]⁺

### Example 9. Synthesis of Compound 9

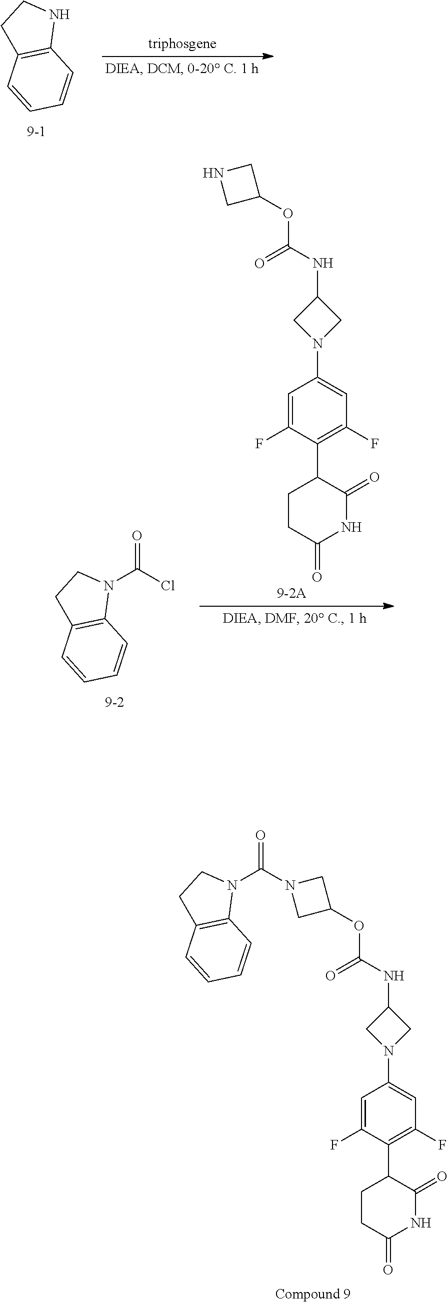 [SMILES: c1ccc2c(c1)CCN2 ; O=C(Cl)N1CCc2ccccc21 ; O=C1CCC(c2c(F)cc(N3CC(NC(=O)OC4CNC4)C3)cc2F)C(=O)N1 ; O=C1CCC(c2c(F)cc(N3CC(NC(=O)OC4CN(C(=O)N5CCc6ccccc65)C4)C3)cc2F)C(=O)N1]

### Step 1. Procedure for Compound 2—indoline-1-carbonyl chloride

To a solution of indoline (30.0 mg, 252 umol, 28.3 uL, 1.00 eq) in dichloromethane (1.00 mL) was added N,N-diisopropylethylamine (48.8 mg, 378 umol, 65.8 uL, 1.50 eq) and triphosgene (112 mg, 378 umol, 1.50 eq) at 0° C. The reaction mixture was stirred at 20° C. for 1 h. The reaction mixture was concentrated under reduced pressure to afford indoline-1-carbonyl chloride (45.0 mg, 248 umol, 98% yield) as a yellow oil.

### Step 2. Procedure for 1-(indoline-1-carbonyl)azetidin-3-yl (1-(4-(2,6-dioxopiperidin-3-yl)-3,5-difluorophenyl)azetidin-3-yl)carbamate

To a solution of azetidin-3-yl (1-(4-(2,6-dioxopiperidin-3-yl)-3,5-difluorophenyl)azetidin-3-yl)carbamate (102 mg, 201 umol, 1.00 eq, trifluoroacetic acid) in dimethyformamide (1.00 mL) was added N,N-diisopropylethylamine (51.9 mg, 401 umol, 69.9 uL, 2.00 eq) and indoline-1-carbonyl chloride (40.1 mg, 221 umol, 1.10 eq). The reaction mixture was stirred at 20° C. for 1 h. The reaction mixture was concentrated under reduced pressure. The residue was purified by Prep-HPLC (column: Phenomenex luna C18 150*25 mm*10 um; mobile phase: [water(formic acid)-acetonitrile]; B %: 33%-63%, 10 min) and lyophilized to afford 1-(indoline-1-carbonyl)azetidin-3-yl-(1-(4-(2,6-dioxopiperidin-3-yl)-3,5-difluorophenyl)azetidin-3-yl)carbamate (36.23 mg, 66.48 umol, 33 yield, 99% purity) as a white solid

¹H NMR (400 MHz, DMSO-d₆) δ=10.86 (s, 1H), 8.14 (br d, J=7.4 Hz, 1H), 7.62 (br d, J=7.9 Hz, 1H), 7.17 (br d, J=7.4 Hz, 1H), 7.10 (t, J=7.6 Hz, 1H), 6.88 (t, J=7.4 Hz, 1H), 6.15 (br d, J=11.3 Hz, 2H), 5.18-4.98 (m, 1H), 4.49-4.36 (m, 1H), 4.35-4.25 (m, 2H), 4.10 (br t, J=7.6 Hz, 2H), 4.03 (br dd, J=5.1, 12.4 Hz, 1H), 3.95-3.84 (m, 4H), 3.66 (br t, J=6.6 Hz, 2H), 3.08 (br t, J=8.4 Hz, 2H), 2.82-2.72 (m, 1H), 2.48-2.46 (m, 1H), 2.11-2.00 (m, 1H), 1.99-1.88 (m, 1H). MS (ESI) m/z. 540.4 [M+H]⁺

### Example 10. Synthesis of Compound 10

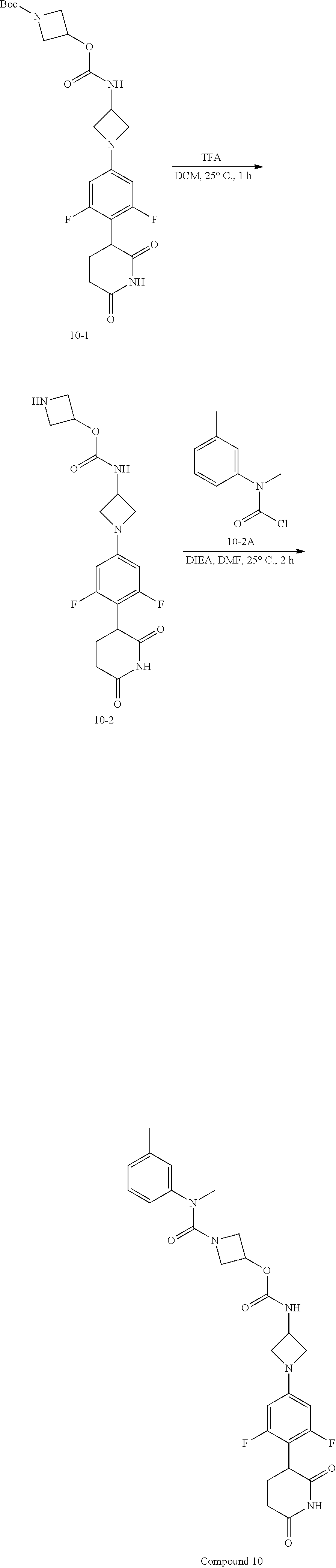 [SMILES: CC(C)(C)OC(=O)N1CC(OC(=O)NC2CN(c3cc(F)c(C4CCC(=O)NC4=O)c(F)c3)C2)C1 ; O=C1CCC(c2c(F)cc(N3CC(NC(=O)OC4CNC4)C3)cc2F)C(=O)N1 ; Cc1cccc(N(C)C(=O)Cl)c1 ; Cc1cccc(N(C)C(=O)N2CC(OC(=O)NC3CN(c4cc(F)c(C5CCC(=O)NC5=O)c(F)c4)C3)C2)c1]

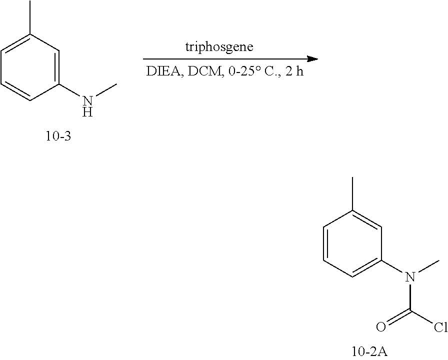

### Step 1. Procedure for Preparation of Compound 2A—methyl(m-tolyl)carbamic chloride

To a solution of N, 3-dimethylaniline (30.0 mg, 247 umol, 30.9 uL, 1.00 eq) in dichloromethane (2.00 mL) was added N,N-diisopropylethylamine (96.0 mg, 743 umol, 129 uL, 3.00 eq) and bis(trichloromethyl) carbonate (95.5 mg, 321 umol, 1.30 eq) at 0° C. The mixture was stirred at 25° C. for 2 h. The mixture was concentrated under reduced pressure to afford methyl(m-tolyl)carbamic chloride (40.0 mg, crude) as colorless oil.

### Step 2. Procedure for Preparation of Compound 2—azetidin-3-yl (1-(4-(2,6-dioxopiperidin-3-yl)-3,5-difluorophenyl)azetidin-3-yl)carbamate

To a solution of tert-butyl 3-(((1-(4-(2,6-dioxopiperidin-3-yl)-3,5-difluorophenyl)azetidin-3-yl)carbamoyl)oxy)azetidine-1-carboxylate (100 mg, 202 umol, 1.00 eq) in dichloromethane (5.00 mL) was added trifluoroacetic acid (1.54 g, 13.5 mmol, 1.00 mL, 66.8 eq). The mixture was stirred at 25° C. for 1 h. The reaction mixture was concentrated under reduced pressure to afford azetidin-3-yl (1-(4-(2,6-dioxopiperidin-3-yl)-3,5-difluorophenyl)azetidin-3-yl)carbamate (70.0 mg, 177 umol, 87% yield) as yellow oil.

### Step 3. Procedure for Preparation of 1-(methyl(m-tolyl)carbamoyl)azetidin-3-yl (1-(4-(2,6-dioxopiperidin-3-yl)-3,5-difluorophenyl)azetidin-3-yl)carbamate

To a solution of azetidin-3-yl (1-(4-(2,6-dioxopiperidin-3-yl)-3,5-difluorophenyl)azetidin-3-yl)carbamate (70.0 mg, 177 umol, 1.00 eq) in dichloromethane (1.00 mL) was added N,N-diisopropylethylamine (45.9 mg, 355 umol, 61.8 uL, 2.00 eq) and methyl(m-tolyl)carbamic chloride (39.1 mg, 213 umol, 1.20 eq). The mixture was stirred at 25° C. for 1 h. The reaction mixture was concentrated under reduced pressure to give a residue. The residue was purified by prep-HPLC (column: Phenomenex luna C18 150*25 mm*10 um; mobile phase: [water (formic acid)-acetonitrile]; B %: 35%-65%, 9 min) to afford 1-(methyl(m-tolyl)carbamoyl)azetidin-3-yl (1-(4-(2,6-dioxopiperidin-3-yl)-3,5-difluorophenyl)azetidin-3-yl) carbamate (20.59 mg, 35.9 umol, 20.2% yield, 94% purity) as a yellow solid.

¹H NMR (400 MHz, DMSO-d₆) δ=10.85 (s, 1H), 8.03 (br d, J=7.5 Hz, 1H), 7.31-7.23 (m, 1H), 7.13-6.99 (m, 3H), 6.13 (br d, J=11.1 Hz, 2H), 4.85-4.74 (m, 1H), 4.43-4.27 (m, 1H), 4.11-4.04 (m, 2H), 4.04-3.98 (m, 1H), 3.80-3.69 (m, 2H), 3.59 (br t, J=6.7 Hz, 2H), 3.35 (br d, J=3.5 Hz, 2H), 3.11 (s, 3H), 2.83-2.72 (m, 1H), 2.47 (br s, 1H), 2.31 (s, 3H), 2.13-2.02 (m, 1H), 1.98-1.88 (m, 1H). MS (ESI) m/z 542.1 [M+H]⁺

### Example 11. Synthesis of Compound 11

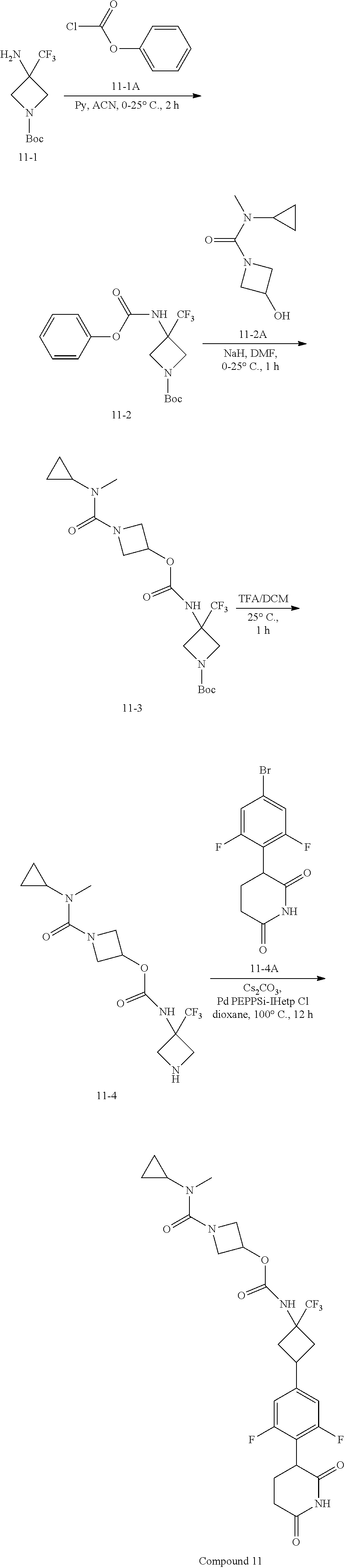

### Step 1. Procedure for Preparation of Compound 2—tert-butyl 3-((phenoxycarbonyl)amino)-3-(trifluoromethyl)azetidine-1-carboxylate

To a solution of tert-butyl 3-amino-3-(trifluoromethyl)azetidine-1-carboxylate (500 mg, 2.08 mmol, 1.00 eq) in acetonitrile (3.00 mL) were added pyridine (494 mg, 6.24 mmol, 504 uL, 3.00 eq) and phenyl carbonochloridate (391 mg, 2.50 mmol, 313 uL, 1.20 eq) at 0° C. The reaction mixture was stirred at 25° C. for 2 h. The reaction mixture was concentrated under reduced pressure to give a residue. The residue was triturated with petroleum ether (20.0 mL) at 25° C. for 30 min to afford tert-butyl 3-((phenoxycarbonyl)amino)-3-(trifluoromethyl)azetidine-1-carboxylate (600 mg, crude) as a white solid.

¹H NMR (400 MHz, DMSO-d₆) δ=9.09 (br s, 1H), 7.44-7.35 (m, 2H), 7.28-7.20 (m, 1H), 7.17 (br d, J=7.9 Hz, 2H), 4.19 (br s, 2H), 4.06 (br d, J=9.6 Hz, 2H), 1.40 (s, 9H).

### Step 2. Procedure for Preparation of Compound 3—tert-butyl 3-((((1-(cyclopropyl(methyl)carbamoyl)azetidin-3-yl)oxy)carbonyl)amino)-3-(trifluoromethyl)azetidine-1-carboxylate

To a solution of tert-butyl 3-(phenoxycarbonylamino)-3-(trifluoromethyl)azetidine-1-carboxylate (500 mg, 1.39 mmol, 1.00 eq) in N,N-dimethylformamide (5.00 mL) were added N-cyclopropyl-3-hydroxy-N-methylazetidine-1-carboxamide (283 mg, 1.67 mmol, 1.20 eq) and sodium hydride (111 mg, 2.78 mmol, 60% purity, 2.00 eq) at 0° C. The reaction was stirred at 25° C. for 1 h. The reaction mixture was quenched with saturated ammonium chloride (5 mL) and extracted with ethyl acetate (2×5 mL). The combined organic layers were washed with brine (10.00 mL), dried over anhydrous sodium sulfate and concentrated under reduce pressure to give a residue. The residue was purified by reverse phase HPLC (C18, 40 g, condition: water/acetonitrile=7/3 to 1/1, 0.1% formic) and lyophilized to afford tert-butyl 3-((((1-(cyclopropyl(methyl)carbamoyl)azetidin-3-yl)oxy)carbonyl)amino)-3-(trifluoromethyl)azetidine-1-carboxylate (277 mg, 635 umol, 46% yield) as a white solid.

¹H NMR (400 MHz, DMSO-d6) δ=8.69 (br s, 1H), 5.08-4.99 (m, 1H), 4.23 (br dd, J=6.8, 9.5 Hz, 2H), 4.14-4.07 (m, 2H), 4.01 (br d, J=9.6 Hz, 2H), 3.85 (br dd, J=3.6, 9.6 Hz, 2H), 2.73 (s, 3H), 2.56 (br dd, J=3.4, 6.8 Hz, 1H), 1.39 (s, 9H), 0.75-0.69 (m, 2H), 0.64-0.59 (m, 2H).

### Step 3. Procedure for Preparation of Compound 4—1-(cyclopropyl(methyl)carbamoyl)azetidin-3-yl (3-(trifluoromethyl)azetidin-3-yl)carbamate

To a solution of tert-butyl 3-((((1-(cyclopropyl(methyl)carbamoyl)azetidin-3-yl)oxy)carbonyl)amino)-3-(trifluoromethyl)azetidine-1-carboxylate (277 mg, 635 umol, 1.00 eq) in dichloromethane (3.00 mL) were added trifluoroacetic acid (924 mg, 8.10 mmol, 0.600 mL, 12.8 eq). The reaction mixture was stirred at 25° C. for 1 h. The reaction mixture was concentrated under reduced pressure to afford 1-(cyclopropyl(methyl)carbamoyl)azetidin-3-yl (3-(trifluoromethyl)azetidin-3-yl)carbamate (213 mg, crude) as yellow oil, which was used to next step directly. MS (ESI) m/z. 337.1 [M+H]⁺

### Step 4. Procedure for Preparation of 1-(cyclopropyl(methyl)carbamoyl)azetidin-3-yl (1-(4-(2,6-dioxopiperidin-3-yl)-3,5-difluorophenyl)-3-(trifluoromethyl)azetidin-3-yl)carbamate

To a solution of 1-(cyclopropyl(methyl)carbamoyl)azetidin-3-yl (3-(trifluoromethyl)azetidin-3-yl)carbamate (213 mg, 633 umol, 1.00 eq) in dioxane (5.00 mL) was added 3-(4-bromo-2,6-difluoro-phenyl)piperidine-2,6-dione (289 mg, 950 umol, 1.50 eq), cesium carbonate (619 mg, 1.90 mmol, 3.00 eq) and [1,3-bis[2,6-bis(1-propylbutyl)phenyl]-4,5-dichloro-imidazol-2-ylidene]-dichloro-(3-chloropyridin-1-ium-1-yl)palladium (61.6 mg, 63.3 umol, 0.100 eq). The reaction mixture was stirred at 100° C. for 12 h under nitrogen atmosphere. The reaction mixture was filtered and the filtrate was concentrated under reduced pressure to give a residue. The residue was purified by column chromatography (SiO₂, petroleum ether/ethyl acetate=1/0 to 0/1) and concentrated under reduced pressure to give a yellow solid. The yellow solid was purified by Prep-HPLC (column: Phenomenex C18 150*25 mm*10 um; mobile phase: [water (ammonium bicarbonate)-acetonitrile]; B %: 48%-78%, 8 min) and lyophilized to afford a crude product. The crude product was triturated with acetonitrile (10.0 ml) at 25° C. for 1 h to afford 1-(cyclopropyl(methyl)carbamoyl)azetidin-3-yl (1-(4-(2,6-dioxopiperidin-3-yl)-3,5-difluorophenyl)-3-(trifluoromethyl)azetidin-3-yl)carbamate (33.53 mg, 59.33 umol, 9.37% yield, 99% purity) as a white solid.

¹H NMR (400 MHz, DMSO-d₆) δ=10.87 (s, 1H), 8.77 (br s, 1H), 6.31 (d, J=10.9 Hz, 2H), 5.12-4.89 (m, 1H), 4.25-4.15 (m, 4H), 4.05 (br d, J=8.6 Hz, 3H), 3.85 (dd, J=3.9, 9.9 Hz, 2H), 2.82-2.76 (m, 1H), 2.72 (s, 3H), 2.58-2.53 (m, 2H), 2.10-2.03 (m, 1H), 1.98-1.90 (m, 1H), 0.75-0.68 (m, 2H), 0.64-0.57 (m, 2H). MS (ESI) m/z. 559.9 [M+H]⁺

### Example 12. Synthesis of Compound 12

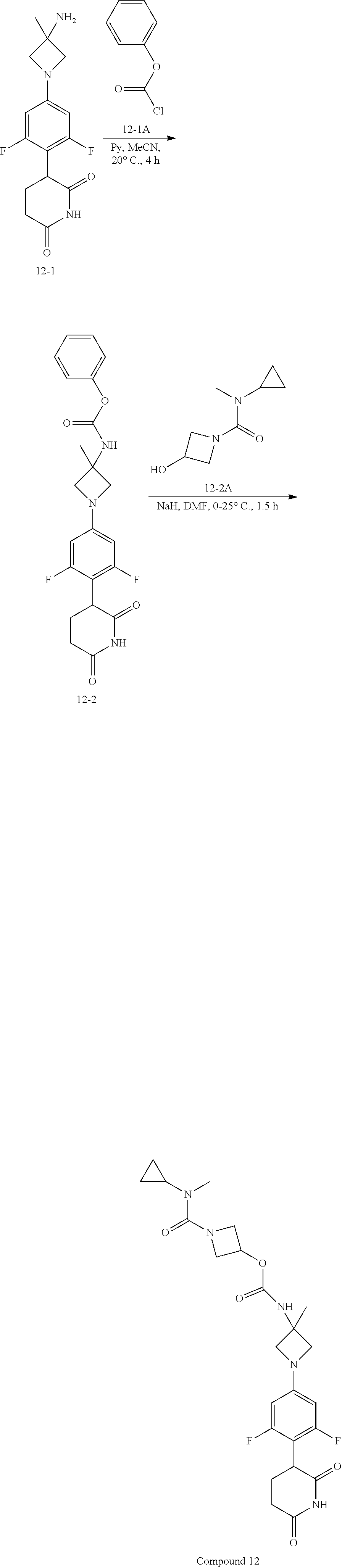 [SMILES: CC1(N)CN(c2cc(F)c(C3CCC(=O)NC3=O)c(F)c2)C1 ; O=C(Cl)Oc1ccccc1 ; CC1(NC(=O)Oc2ccccc2)CN(c2cc(F)c(C3CCC(=O)NC3=O)c(F)c2)C1 ; CN(C(=O)N1CC(O)C1)C1CC1 ; CN(C(=O)N1CC(OC(=O)NC2(C)CN(c3cc(F)c(C4CCC(=O)NC4=O)c(F)c3)C2)C1)C1CC1]

### Step 1. Procedure for Preparation of Compound 2—phenyl (1-(4-(2,6-dioxopiperidin-3-yl)-3,5-difluorophenyl)-3-methylazetidin-3-yl)carbamate

To a solution of 3-(4-(3-amino-3-methylazetidin-1-yl)-2,6-difluorophenyl)piperidine-2,6-dione (400 mg, 1.29 mmol, 1.00 eq) in acetonitrile (2.00 mL) was added 4-dimethylaminopyridine (15.8 mg, 129 umol, 0.100 eq) and triethylamine (393 mg, 3.88 mmol, 540 uL, 3.00 eq) and phenyl carbonochloridate (243 mg, 1.55 mmol, 194 uL, 1.20 eq). The mixture was stirred at 20° C. for 4 h. The reaction mixture was concentrated under reduced pressure to give a residue. The residue was purified by reverse phase chromatography (C18, 40 g; condition: water/acetonitrile=100:0 to 0:100, 0.1% formic acid) to afford phenyl (1-(4-(2,6-dioxopiperidin-3-yl)-3,5-difluorophenyl)-3-methylazetidin-3-yl)carbamate (62.0 mg, 144 umol, 11% yield) as a white solid.

¹H NMR (400 MHz, DMSO-d₆) δ=10.86 (s, 1H), 8.39 (s, 1H), 7.37 (br d, J=7.9 Hz, 2H), 7.26-7.18 (m, 2H), 7.13 (br s, 1H), 6.18 (br d, J=11.1 Hz, 2H), 4.03 (br dd, J=5.1, 12.9 Hz, 1H), 3.97 (br d, J=7.9 Hz, 2H), 3.76 (br d, J=7.6 Hz, 2H), 2.81-2.74 (m, 1H), 2.63-2.54 (m, 1H), 2.10-2.04 (m, 1H), 1.96-1.90 (m, 1H), 1.57 (s, 3H)

### Step 2. Procedure for Preparation of 1-(cyclopropyl(methyl)carbamoyl)azetidin-3-yl(1-(4-(2,6-dioxopiperidin-3-yl)-3,5-difluorophenyl)-3-methylazetidin-3-yl)carbamate

To a solution of N-cyclopropyl-3-hydroxy-N-methylazetidine-1-carboxamide (28.5 mg, 168. umol, 1.20 eq) in dimethylformamide (1.00 mL) was added sodium hydride (67.1 mg, 279 umol, 60% purity, 2.00 eq) at 0° C. The mixture was stirred at 25° C. for 0.5 h. To a result solution was added phenyl (1-(4-(2,6-dioxopiperidin-3-yl)-3,5-difluorophenyl)-3-methylazetidin-3-yl)carbamate (60.0 mg, 140 umol, 1 eq). The mixture was stirred at 25° C. for 1 h. The reaction mixture was quench with water (1.00 mL) and concentrated under reduce pressure to give a residue. The residue was purified by Prep-HPLC (Phenomenex luna C18 150*25 mm*10 um; mobile phase: [water(formic acid)-acetonitrile]; B %: 25%-55%, 10 min) and lyophilized to afford 1-(cyclopropyl(methyl)carbamoyl)azetidin-3-yl (1-(4-(2,6-dioxopiperidin-3-yl)-3,5-difluorophenyl)-3-methylazetidin-3-yl)carbamate (13.65 mg, 24.50 umol, 17.54% yield, 96% purity, formic acid) as a white solid.

¹H NMR (400 MHz, DMSO-d₆) δ=10.85 (s, 1H), 8.52-8.43 (m, 1H), 8.17-7.81 (m, 1H), 6.14 (d, J=11.1 Hz, 2H), 5.16-4.85 (m, 1H), 4.22 (dd, J=6.8, 9.6 Hz, 2H), 4.03 (br dd, J=4.9, 12.2 Hz, 1H), 3.88 (br d, J=7.5 Hz, 2H), 3.81 (dd, J=3.9, 9.6 Hz, 2H), 3.71 (br d, J=7.4 Hz, 2H), 2.83-2.74 (m, 1H), 2.72 (s, 3H), 2.60-2.55 (m, 1H), 2.55-2.53 (m, 1H), 2.13-2.01 (m, 1H), 1.97-1.85 (m, 1H), 1.50 (s, 3H), 0.78-0.66 (m, 2H), 0.65-0.55 (m, 2H). MS (ESI) m/z. 506.3 [M+H]⁺

### Example 13. Synthesis of Compound 13

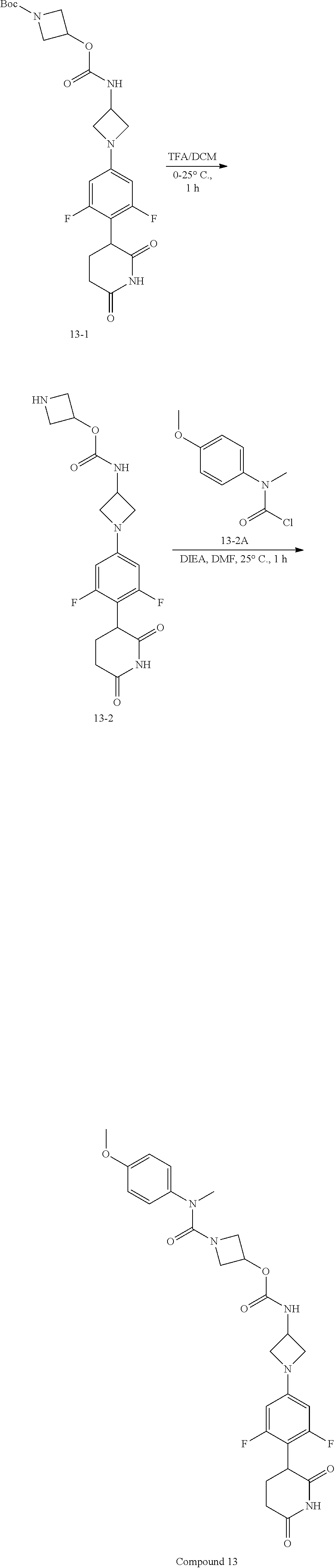 [SMILES: CC(C)(C)OC(=O)N1CC(OC(=O)NC2CN(c3cc(F)c(C4CCC(=O)NC4=O)c(F)c3)C2)C1 ; O=C1CCC(c2c(F)cc(N3CC(NC(=O)OC4CNC4)C3)cc2F)C(=O)N1 ; COc1ccc(N(C)C(=O)Cl)cc1 ; COc1ccc(N(C)C(=O)N2CC(OC(=O)NC3CN(c4cc(F)c(C5CCC(=O)NC5=O)c(F)c4)C3)C2)cc1]

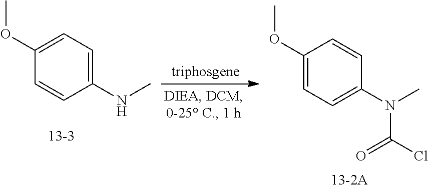

### Step 1. Procedure for Compound 2A—(4-methoxyphenyl)(methyl)carbamic chloride

To mixture of 4-methoxy-N-methyl aniline (45.0 mg, 328 umol, 1.00 eq) and N,N-diisopropylethyl amine (84.8 mg, 656 umol, 114 uL, 2.00 eq) in dichloromethane (2.00 mL) was added triphosgene (195 mg, 656 umol, 2.00 eq) at 0° C. The mixture was stirred at 25° C. for 1 h. The mixture was concentrated under reduced pressure to give a (4-methoxyphenyl)(methyl)carbamic chloride (65.0 mg, crude) as yellow oil.

### Step 2. Procedure for Compound 2—azetidin-3-yl (1-(4-(2,6-dioxopiperidin-3-yl)-3,5-difluorophenyl)azetidin-3-yl)carbamate

To solution of tert-butyl 3-(((1-(4-(2,6-dioxopiperidin-3-yl)-3,5-difluorophenyl)azetidin-3-yl) carbamoyl)oxy)azetidine-1-carboxylate (126 mg, 255 umol, 1.00 eq) in trifluoroacetic acid (1.00 mL) and dichloromethane (5.00 mL) at 0° C. The mixture was stirred at 25° C. for 1 h. The mixture was concentrated under reduced pressure to afford azetidin-3-yl (1-(4-(2,6-dioxopiperidin-3-yl)-3,5-difluorophenyl)azetidin-3-yl)carbamate (100 mg, crude) as yellow oil. MS (ESI) m/z. 395.2 [M+H]⁺

### Step 3. Procedure for 3-(2,6-dichloro-4-(3-((5-(spiro[3.3]heptan-2-yl)-1,3,4-oxadiazol-2-yl)amino)azetidin-1-yl)phenyl)piperidine-2,6-dione

To a solution of azetidin-3-yl (1-(4-(2,6-dioxopiperidin-3-yl)-3,5-difluorophenyl)azetidin-3-yl)carbamate (100 mg, 254 umol, 1.00 eq) in dimethyl formamide (2.00 mL) was added N,N-diisopropylethylamine (65.5 mg, 507 umol, 88.3 uL, 2.00 eq) and (4-methoxyphenyl)(methyl)carbamic chloride (60.7 mg, 304 umol, 1.20 eq) at 25° C. The mixture was stirred at 25° C. for 1 h. The mixture was concentrated under reduced pressure to give a residue. The residue was purified by column chromatography (SiO₂, petroleum ether/ethyl acetate=1/1) to afford 1-((4-methoxyphenyl)(methyl)carbamoyl)azetidin-3-yl(1-(4-(2,6-dioxopiperidin-3-yl)-3,5-difluorophenyl) azetidin-3-yl)carbamate (35.23 mg, 60.0 umol, 23% yield, 95% purity) as a white solid

¹H NMR (400 MHz, DMSO-d₆) δ=10.85 (s, 1H), 8.03 (br d, J=7.4 Hz, 1H), 7.18 (br d, J=8.8 Hz, 2H), 6.94 (d, J=8.8 Hz, 2H), 6.13 (br d, J=11.0 Hz, 2H), 4.85-4.72 (m, 1H), 4.43-4.25 (m, 1H), 4.08-4.03 (m, 2H), 4.01 (br d, J=5.0 Hz, 1H), 3.76 (s, 3H), 3.72-3.65 (m, 2H), 3.59 (br t, J=6.6 Hz, 2H), 3.31-3.29 (m, 2H), 3.07 (s, 3H), 2.81-2.72 (m, 1H), 2.40 (br s, 1H), 2.10-2.01 (m, 1H), 1.96-1.91 (m, 1H).

¹H NMR (400 MHz, DMSO-d₆) δ=7.16 (d, J=9.0 Hz, 2H), 6.93 (d, J=9.0 Hz, 2H), 6.11 (br d, J=11.0 Hz, 2H), 4.79-4.72 (m, 1H), 4.39-4.29 (m, 1H), 4.04 (br t, J=8.0 Hz, 2H), 3.99 (br d, J=4.6 Hz, 1H), 3.74 (s, 3H), 3.71-3.66 (m, 2H), 3.58 (br s, 2H), 3.30 (br dd, J=3.4, 9.7 Hz, 2H), 3.05 (s, 3H), 2.80-2.70 (m, 1H), 2.60-2.53 (m, 1H), 2.10-1.99 (m, 1H), 1.97-1.89 (m, 1H). MS (ESI) m/z. 558.4 [M+H]⁺

### Example 14. Synthesis of Compound 14

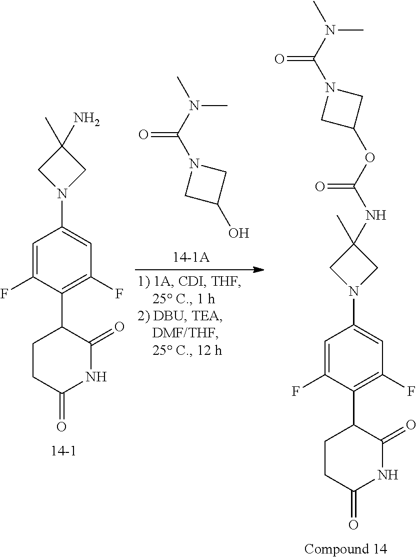

### Step 1. Procedure or Preparation of 1-(dimethylcarbamoyl)azetidin-3-yl (1-(4-(2,6-dioxopiperidin-3-yl)-3,5-difluorophenyl)-3-methylazetidin-3-yl)carbamate

To a solution of 3-hydroxy-N,N-dimethylazetidine-1-carboxamide (53.0 mg, 368 umol, 1.00 eq) in tetrahydrofuran (1.00 mL) was added di(1H-imidazol-1-yl)methanone (71.5 mg, 441 umol, 1.20 eq). The mixture was stirred at 25° C. for 1 h. The resulting solution was added to a solution of 3-(4-(3-amino-3-methylazetidin-1-yl)-2,6-difluorophenyl)piperidine-2,6-dione (148 mg, 366 umol, 1.00 eq, mesylate), triethylamine (56.0 mg, 549 umol, 76.5 uL, 1.50 eq) and 2,3,4,6,7,8,9,10-octahydropyrimido[1,2-a]azepine (83.6 mg, 549 umol, 82.8 uL, 1.50 eq) in tetrahydrofuran (1.00 mL) and dimethylformamide (1.00 mL). The reaction mixture was stirred at 25° C. for 12 h. The reaction mixture was concentrated under reduced pressure to give a residue. The residue was purified by reverse phase chromatography (C18, 40 g; condition: water/acetonitrile=100:0 to 0:100, 0.1% formic acid) to afford 1-(dimethylcarbamoyl)azetidin-3-yl (1-(4-(2,6-dioxopiperidin-3-yl)-3,5-difluorophenyl)-3-methylazetidin-3-yl)carbamate (6.13 mg, 11.76 umol, 3% yield, 92% purity) as an off-white solid.

¹H NMR (400 MHz, DMSO-d₆) δ=10.85 (s, 1H), 8.41-8.36 (m, 1H), 8.04-7.96 (m, 1H), 6.19-6.10 (m, 2H), 5.04-4.92 (m, 1H), 4.20-4.10 (m, 2H), 4.06-4.00 (m, 1H), 3.88 (br d, J=7.5 Hz, 2H), 3.78-3.69 (m, 4H), 2.85-2.77 (m, 1H), 2.74 (s, 6H), 2.59-2.56 (m, 1H), 2.09-2.05 (m, 1H), 1.96-1.91 (m, 1H), 1.50 (s, 3H). MS (ESI) m/z. 480.4 [M+H]⁺

### Example 15. Synthesis of Compound 15

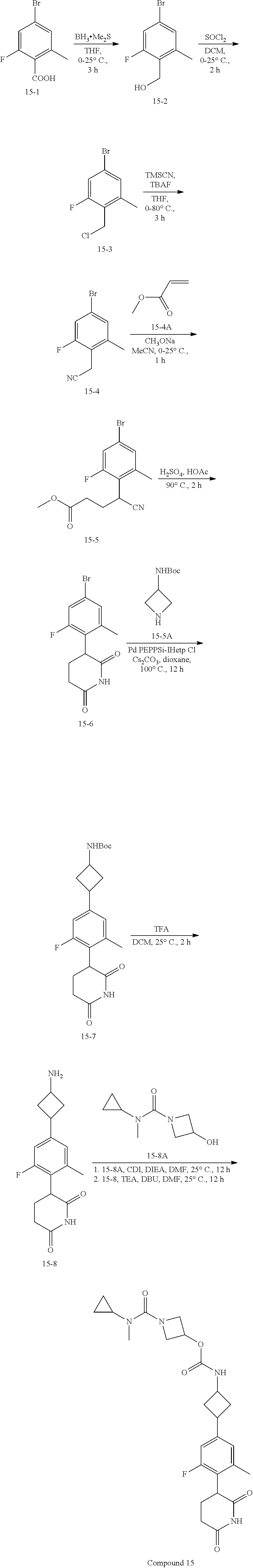

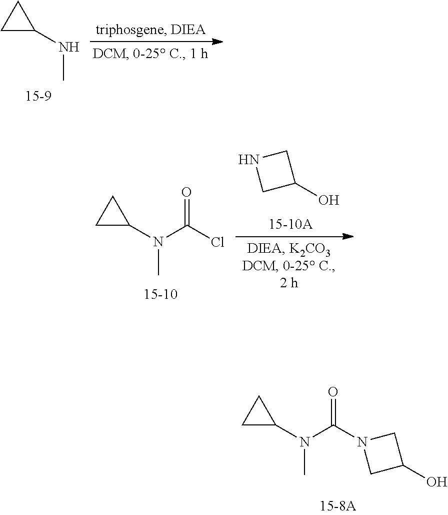

### Step 1. Procedure for Preparation of Compound 10—cyclopropyl(methyl)carbamic chloride

To a solution of N-methylcyclopropanamine (3.00 g, 42.2 mmol, 1.00 eq) in dichloromethane (20.0 mL) were added bis(trichloromethyl) carbonate (20.0 g, 67.6 mmol, 1.60 eq) and N,N-diisopropylethylamine (10.9 g, 84.4 mmol, 14.7 mL, 2.00 eq) at 0° C. The mixture was stirred at 25° C. for 1 h. The reaction mixture was concentrated under reduced pressure to afford cyclopropyl(methyl)carbamic chloride (5.00 g, crude) as a brown solid.

### Step 2. Procedure for Preparation of Compound 8A—N-cyclopropyl-3-hydroxy-N-methylazetidine-1-carboxamide

To a solution of azetidin-3-ol (4.51 g, 41.1 mmol, 1.10 eq, hydrochloride) in dichloromethane (50.0 mL) were added N,N-diisopropylethylamine (9.68 g, 74.8 mmol, 13.0 mL, 2.00 eq), potassium carbonate (5.17 g, 37.4 mmol, 1.00 eq) at 0° C., then cyclopropyl(methyl)carbamic chloride (5.00 g, 37.4 mmol, 1.00 eq) was added into the mixture. The mixture was stirred at 25° C. for 2 h. The mixture was filtered and the filtrate was concentrated under reduced pressure to give a residue. The residue was purified by reverse phase chromatography (C18, 40 g; condition: water/acetonitrile=100:0 to 0:100, 0.1% formic acid) and lyophilized to afford N-cyclopropyl-3-hydroxy-N-methylazetidine-1-carboxamide (1.00 g, 5.88 mmol, 16% yield) as yellow oil.

¹H NMR (400 MHz, DMSO-d₆) δ=5.51 (d, J=6.1 Hz, 1H), 4.40-4.30 (m, 1H), 4.11-4.03 (m, 2H), 3.66 (dd, J=4.8, 8.9 Hz, 2H), 2.71 (s, 3H), 2.54 (d, J=3.5 Hz, 1H), 0.74-0.67 (m, 2H), 0.66-0.56 (m, 2H).

### Step 3. Procedure for Preparation of Compound 2—(4-bromo-2-fluoro-6-methylphenyl)methanol

To a solution of 4-bromo-2-fluoro-6-methylbenzoic acid (2.00 g, 8.58 mmol, 1.00 eq) in tetrahydrofuran (20.0 mL) was added borane dimethyl sulfide complex (10.0 M, 1.72 mL, 2.00 eq) at 0° C. The mixture was stirred at 25° C. for 3 h. The reaction mixture was quenched by addition methanol (20 mL) at 0° C. and concentrated under reduced pressure to afford (4-bromo-2-fluoro-6-methylphenyl)methanol (1.81 g, 8.26 mmol, 96% yield) as a white solid.

### Step 4. Procedure for Preparation of Compound 3—5-bromo-2-(chloromethyl)-1-fluoro-3-methylbenzene

To a solution of (4-bromo-2-fluoro-6-methylphenyl)methanol (1.80 g, 8.22 mmol, 1.00 eq) in dichloromethane (20.0 mL) was added sulfurous dichloride (1.96 g, 16.4 mmol, 1.19 mL, 2.00 eq) at 0° C. The mixture was stirred at 25° C. for 2 h. The reaction mixture was concentrated under reduced pressure to give a residue. The residue was purified by column chromatography (SiO₂, petroleum ether/ethyl acetate=80/1) to afford 5-bromo-2-(chloromethyl)-1-fluoro-3-methylbenzene (1.53 g, 6.45 mmol, 78% yield) as light yellow oil.

¹H NMR (400 MHz, CDCl₃) δ=7.19 (s, 1H), 7.14 (d, J=9.1 Hz, 1H), 4.62 (s, 2H), 2.43 (s, 3H).

### Step 5. Procedure for Preparation of Compound 4—2-(4-bromo-2-fluoro-6-methylphenyl)acetonitrile

To a solution of 5-bromo-2-(chloromethyl)-1-fluoro-3-methylbenzene (1.53 g, 6.45 mmol, 1.00 eq) in acetonitrile (15.0 mL) were added tetrabutylammonium fluoride (1.00 M, 19.3 mL, 3.00 eq) and trimethylsilanecarbonitrile (1.92 g, 19.3 mmol, 2.42 mL, 3.00 eq) at 0° C. The mixture was stirred at 80° C. for 3 h. The reaction mixture was purified by column chromatography (SiO₂, petroleum ether/ethyl acetate=80/1) to afford 2-(4-bromo-2-fluoro-6-methylphenyl) acetonitrile (1.50 g, crude) as colorless oil.

### Step 6. Procedure for Preparation of Compound 5—methyl 4-(4-bromo-2-fluoro-6-methylphenyl)-4-cyanobutanoate

To a solution of 2-(4-bromo-2-fluoro-6-methylphenyl) acetonitrile (1.20 g, 5.26 mmol, 1.00 eq) in tetrahydrofuran (10.0 mL) were added sodium methoxide (56.8 mg, 1.05 mmol, 0.200 eq) and methyl acrylate (498 mg, 5.79 mmol, 521 uL, 1.10 eq) at 0° C. The mixture was stirred at 25° C. for 1 h. The mixture was quenched with saturated ammonium chloride aqueous solution (10 mL) and extracted with ethyl acetate (3×50 mL). The combined organic layers were dried over anhydrous sodium sulfate, filtered and concentrated under reduced pressure. The residue was purified by column chromatography (SiO₂, petroleum ether/ethyl acetate=10/1 to 1/1) to afford methyl 4-(4-bromo-2-fluoro-6-methylphenyl)-4-cyanobutanoate (1.30 g, 4.15 mmol, 87% yield) as colorless oil.

### Step 7. Procedure for Preparation of Compound 6—3-(4-bromo-2-fluoro-6-methylphenyl)piperidine-2,6-dione

To a solution of methyl 4-(4-bromo-2-fluoro-6-methylphenyl)-4-cyanobutanoate (1.30 g, 4.15 mmol, 1.00 eq) in acetic acid (10 mL) was added sulfuric acid (1.84 g, 18.7 mmol, 1.00 mL, 4.52 eq). The mixture was stirred at 90° C. for 2 h. The reaction mixture was concentrated under reduced pressure to give a residue. The residue was triturated with water and filtered. The filter cake was washed with petroleum ether and concentrated under reduced pressure to afford 3-(4-bromo-2-fluoro-6-methylphenyl)piperidine-2,6-dione (1.06 g, 3.55 mmol, 85% yield) as a white solid.

¹H NMR (400 MHz, DMSO-d₆) δ=10.90 (s, 1H), 7.45-7.27 (m, 2H), 4.13 (br dd, J=5.2, 11.9 Hz, 1H), 2.88-2.71 (m, 1H), 2.54 (br d, J=3.1 Hz, 1H), 2.33 (s, 3H), 2.13-1.93 (m, 2H).

### Step 8. Procedure for Preparation of Compound 7—tert-butyl (1-(4-(2,6-dioxopiperidin-3-yl)-3-fluoro-5-methylphenyl)azetidin-3-yl)carbamate

A mixture of tert-butyl azetidin-3-ylcarbamate (275 mg, 1.60 mmol, 1.20 eq), 3-(4-bromo-2-fluoro-6-methylphenyl)piperidine-2,6-dione (400 mg, 1.33 mmol, 1.00 eq), [1,3-bis[2,6-bis(1-propylbutyl)phenyl]-4,5-dichloro-imidazol-2-ylidene]-dichloro-(3-chloropyridin-1-ium-1-yl)palladium (129 mg, 133 umol, 0.100 eq), cesium carbonate (1.30 g, 4.00 mmol, 3.00 eq) in dioxane (10.0 mL) was degassed and purged with nitrogen for 3 times. The mixture was stirred at 100° C. for 12 h under nitrogen atmosphere. The reaction mixture was concentrated under reduced pressure to give a residue. The residue was purified by reverse phase chromatography (C18, 40 g; condition: water/acetonitrile=100:0 to 0:100, 0.1% formic acid) and lyophilized to afford tert-butyl (1-(4-(2,6-dioxopiperidin-3-yl)-3-fluoro-5-methylphenyl)azetidin-3-yl)carbamate (251 mg, 641 umol, 48% yield) as a white solid.

¹H NMR (400 MHz, DMSO-d₆) δ=10.76 (s, 1H), 7.53 (br d, J=7.4 Hz, 1H), 6.17-6.01 (m, 2H), 4.45-4.33 (m, 1H), 4.10-4.02 (m, 2H), 3.99-3.89 (m, 1H), 3.54 (br t, J=6.8 Hz, 2H), 2.81-2.71 (m, 1H), 2.59-2.53 (m, 1H), 2.22 (s, 3H), 2.09-2.00 (m, 1H), 1.95-1.87 (m, 1H), 1.42-1.37 (m, 9H).

### Step 9. Procedure for Preparation of Compound 8—3-(4-(3-aminoazetidin-1-yl)-2-fluoro-6-methylphenyl)piperidine-2,6-dione

To a solution of tert-butyl (1-(4-(2,6-dioxopiperidin-3-yl)-3-fluoro-5-methylphenyl)azetidin-3-yl)carbamate (130 mg, 332 umol, 1.00 eq) in dichloromethane (2.50 mL) was added trifluoroacetic acid (1.25 g, 10.9 mmol, 812 uL, 33.0 eq). The mixture was stirred at 25° C. for 2 h. The reaction mixture was concentrated under reduced pressure to afford 3-(4-(3-aminoazetidin-1-yl)-2-fluoro-6-methylphenyl)piperidine-2,6-dione (90.0 mg, crude) as a white solid. MS (ESI) m/z 292.2 [M+H]⁺

### Step 10. Procedure for Preparation of 1-(cyclopropyl(methyl)carbamoyl)azetidin-3-yl (1-(4-(2,6-dioxopiperidin-3-yl)-3-fluoro-5-methylphenyl)azetidin-3-yl)carbamate

To a solution of N-cyclopropyl-3-hydroxy-N-methylazetidine-1-carboxamide (100 mg, 587 umol, 1.00 eq) in tetrahydrofuran (5.00 mL) was added di(1H-imidazol-1-yl)methanone (100 mg, 616 umol, 1.05 eq) at 25° C. The mixture was stirred at 25° C. for 12 h. The resulting solution was added to a mixture of 3-(4-(3-aminoazetidin-1-yl)-2-fluoro-6-methylphenyl)piperidine-2,6-dione (89.0 mg, 305 umol, 1.00 eq), triethylamine (30.9 mg, 305 umol, 42.5 uL, 1.00 eq) and 2,3,4,6,7,8,9,10-octahydropyrimido[1,2-a]azepine (46.5 mg, 305 umol, 46.0 uL, 1.00 eq) in dimethylformamide (5.00 mL). The mixture was stirred at 25° C. for 12 h. The mixture was concentrated under reduced pressure to give a residue. The residue was purified by reverse phase chromatography (C18, 40 g; condition: water/acetonitrile=100:0 to 0:100, 0.1% formic acid) and lyophilized to afford 1-(cyclopropyl(methyl)carbamoyl)azetidin-3-yl (1-(4-(2,6-dioxopiperidin-3-yl)-3-fluoro-5-methylphenyl)azetidin-3-yl)carbamate (24.67 mg, 47.5 umol, 16% yield, 94% purity) as a white solid.

¹H NMR (400 MHz, DMSO-d₆) δ=10.92-10.64 (m, 1H), 8.08 (br d, J=7.4 Hz, 1H), 6.21-5.97 (m, 2H), 5.01 (br s, 1H), 4.41 (br d, J=6.9 Hz, 1H), 4.22 (dd, J=6.8, 9.6 Hz, 2H), 4.11-4.03 (m, 2H), 3.97-3.89 (m, 1H), 3.81 (br dd, J=3.9, 9.8 Hz, 2H), 3.58 (br t, J=6.7 Hz, 2H), 2.80-2.74 (m, 1H), 2.72 (s, 3H), 2.58 (br d, J=3.9 Hz, 2H), 2.21 (s, 3H), 2.01 (dt, J=10.0, 12.4 Hz, 1H), 1.90 (br dd, J=4.4, 6.4 Hz, 1H), 0.72 (br d, J=5.0 Hz, 2H), 0.66-0.56 (m, 2H). MS (ESI) m/z 488.4 [M+H]⁺

### Example 16. Synthesis of Compound 16

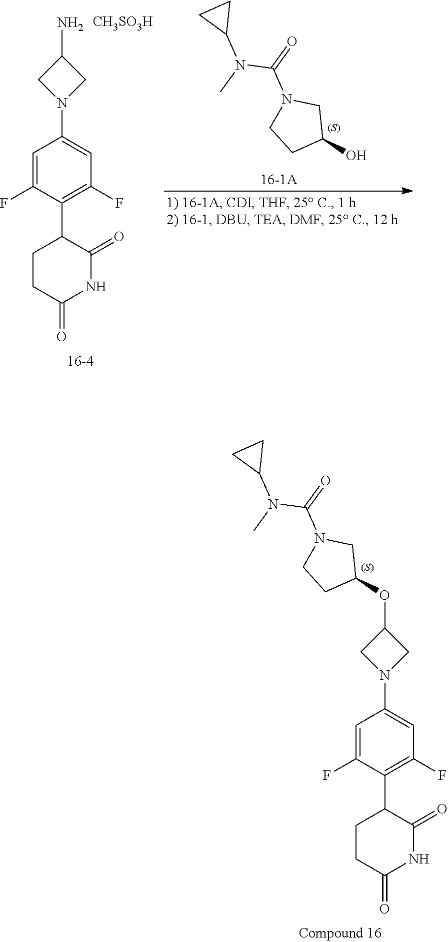

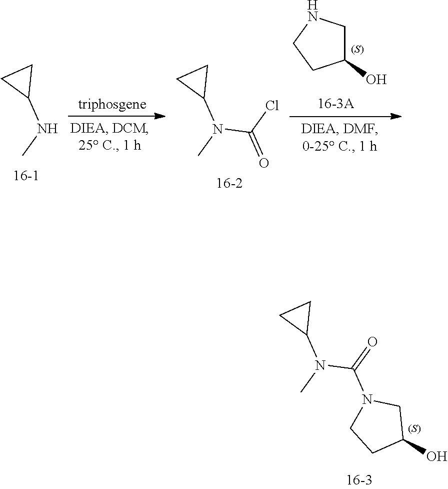

### Step 1. Procedure for Preparation of Compound 2—cyclopropyl(methyl)carbamic chloride

To a solution of N-methylcyclopropanamine (400 mg, 5.62 mmol, 1.00 eq) in dichloromethane (10.0 mL) were added bis(trichloromethyl) carbonate (2.67 g, 9.00 mmol, 1.60 eq) and N,N-diisopropylethylamine (1.45 g, 11.25 mmol, 1.96 mL, 2 eq) at 25° C. The mixture was stirred at 25° C. for 1 h. The mixture was concentrated under reduced pressure to afford cyclopropyl(methyl)carbamic chloride (750 mg, 5.61 mmol, 99% yield) as a red brown solid.

### Step 2. Procedure for Preparation of Compound 3—(S)—N-cyclopropyl-3-hydroxy-N-methylpyrrolidine-1-carboxamide

To a solution of (S)-pyrrolidin-3-ol 1 (300 mg, 3.44 mmol, 278 uL, 1.00 eq) in dichloromethane (9.00 mL) were added N,N-diisopropylethylamine (890 mg, 6.89 mmol, 1.20 mL, 2.00 eq) and cyclopropyl(methyl)carbamic chloride (736 mg, 5.51 mmol, 1.60 eq) at 0° C. The mixture was stirred at 25° C. for 1 h. The reaction mixture was concentrated under reduced pressure to give a residue. The residue was purified by reverse phase chromatography (C18, 40 g; condition: water/acetonitrile=100:0 to 0:100, 0.1% formic acid) to afford (S)—N-cyclopropyl-3-hydroxy-N-methylpyrrolidine-1-carboxamide (200 mg, 1.09 mmol, 32% yield) as colorless oil.

¹H NMR (400 MHz, DMSO-d₆) δ=4.84 (d, J=3.4 Hz, 1H), 4.26-4.13 (m, 1H), 3.49-3.41 (m, 2H), 3.26 (br s, 1H), 3.15-3.08 (m, 1H), 2.72-2.65 (m, 3H), 2.59 (tt, J=3.6, 6.8 Hz, 1H), 1.85-1.75 (m, 1H), 1.74-1.63 (m, 1H), 0.70-0.59 (m, 2H), 0.55-0.46 (m, 2H).

### Step 3. Procedure for Preparation of (S)-1-(cyclopropyl(methyl)carbamoyl)pyrrolidin-3-yl (1-(4-(2,6-dioxopiperidin-3-yl)-3,5-difluorophenyl)azetidin-3-yl)carbamate

To a solution of (S)—N-cyclopropyl-3-hydroxy-N-methylpyrrolidine-1-carboxamide (120 mg, 651 umol, 1.00 eq) in tetrahydrofuran (5.00 mL) was added di(1H-imidazol-1-yl)methanone (127 mg, 782 umol, 1.20 eq). The mixture was stirred at 25° C. for 1 h. The resulting solution was added to a solution of 3-(4-(3-aminoazetidin-1-yl)-2,6-difluorophenyl)piperidine-2,6-dione (150 mg, 383 umol, 1.00 eq, mesylate), triethylamine (38.8 mg, 383 umol, 53.3 uL, 1.00 eq) and 2,3,4,6,7,8,9,10-octahydropyrimido[1,2-a]azepine (58.3 mg, 383 umol, 57.8 uL, 1.00 eq) in dimethylformamide (1.00 mL). The mixture was stirred at 25° C. for 12 h. The reaction mixture was concentrated under reduced pressure to give a residue. The residue was reverse phase chromatography (C18, 40 g; condition: water/acetonitrile=100:0 to 0:100, 0.1% formic acid condition) to afford to (S)-1-(cyclopropyl(methyl)carbamoyl)pyrrolidin-3-yl (1-(4-(2,6-dioxopiperidin-3-yl)-3,5-difluorophenyl)azetidin-3-yl)carbamate (35.54 mg, 68.90 umol, 18% yield, 98% purity) as a white solid.

¹H NMR (400 MHz, DMSO-d₆) δ=10.85 (s, 1H), 7.95 (br d, J=7.3 Hz, 1H), 6.13 (d, J=11.0 Hz, 2H), 5.08 (br s, 1H), 4.47-4.38 (m, 1H), 4.08 (br t, J=7.6 Hz, 2H), 4.05-3.98 (m, 1H), 3.67-3.60 (m, 3H), 3.47-3.38 (m, 2H), 3.29 (br s, 1H), 2.84-2.75 (m, 1H), 2.70 (s, 3H), 2.61 (td, J=3.2, 6.8 Hz, 1H), 2.18-1.74 (m, 5H), 0.75-0.68 (m, 1H), 0.67-0.60 (m, 1H), 0.56-0.50 (m, 2H). MS (ESI) m/z 506.1 [M+H]⁺

### Example 17. Synthesis of Compound 17

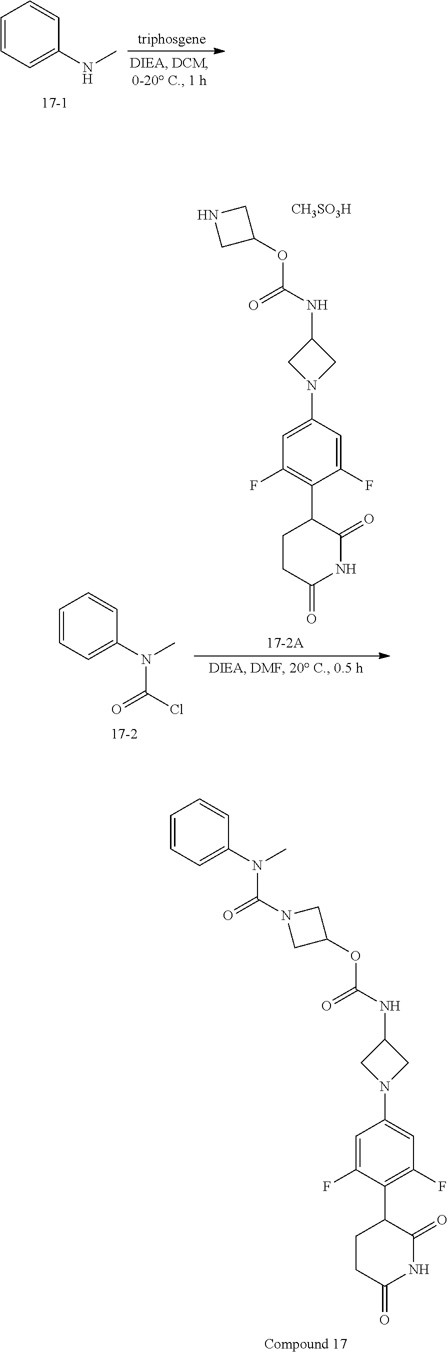

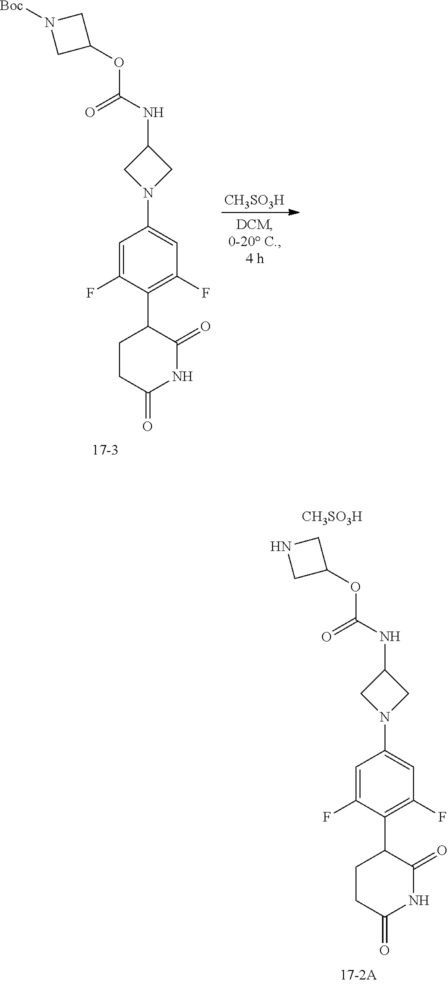 [SMILES: CC(C)(C)OC(=O)N1CC(OC(=O)NC2CN(c3cc(F)c(C4CCC(=O)NC4=O)c(F)c3)C2)C1 ; "CH3SO3H" ; O=C1CCC(c2c(F)cc(N3CC(NC(=O)OC4CNC4)C3)cc2F)C(=O)N1 ; "CH3SO3H"]

### Step 1. Procedure for Preparation of Compound 2A—azetidin-3-yl (1-(4-(2,6-dioxopiperidin-3-yl)-3,5-difluorophenyl)azetidin-3-yl)carbamate

To a solution of tert-butyl 3-(((1-(4-(2,6-dioxopiperidin-3-yl)-3,5-difluorophenyl)azetidin-3-yl)carbamoyl) oxy)azetidine-1-carboxylate (80.0 mg, 162 umol, 1.00 eq) in dichloromethane (1.00 mL) was added methanesulfonic acid (46.6 mg, 485 umol, 34.6 uL, 3.00 eq) at 0° C. The reaction mixture was stirred at 20° C. for 4 h. The reaction mixture was concentrated under reduced pressure to afford azetidin-3-yl (1-(4-(2,6-dioxopiperidin-3-yl)-3,5-difluorophenyl)azetidin-3-yl)carbamate (79.0 mg, crude, mesylate) as yellow oil. MS (ESI) m/z 395.2 [M+H]⁺

### Step 2. Procedure for Preparation of Compound 2—methyl(phenyl)carbamic chloride

To a solution of N-methylaniline (25.0 mg, 233 umol, 25.3 uL, 1.00 eq) in dichloromethane (0.300 mL) were added bis(trichloromethyl) carbonate (69.2 mg, 233 umol, 1.0 eq) and N,N-diisopropylethylamine (60.3 mg, 467 umol, 81.3 uL, 2.00 eq) at 0° C. The reaction mixture was stirred at 20° C. for 1 h. The reaction mixture was concentrated under reduced pressure to afford methyl(phenyl)carbamic chloride (39.0 mg, crude) as yellow oil.

### Step 3. Procedure for Preparation of 1-(methyl(phenyl)carbamoyl)azetidin-3-yl (1-(4-(2,6-dioxopiperidin-3-yl)-3,5-difluorophenyl)azetidin-3-yl)carbamate

To a solution of azetidin-3-yl (1-(4-(2,6-dioxopiperidin-3-yl)-3,5-difluorophenyl)azetidin-3-yl)carbamate (79.0 mg, 161 umol, 1.00 eq, mesylate) in dimethylformamide (1.00 mL) were added N,N-diisopropylethylamine (62.5 mg, 483 umol, 84.2 uL, 3.00 eq) and methyl(phenyl)carbamic chloride (32.8 mg, 193 umol, 1.20 eq). The reaction mixture was stirred at 20° C. for 0.5 h. The reaction mixture was filtered. The filtrate was purified by Prep-HPLC (column: Phenomenex luna C18 150*25 mm*10 um; mobile phase: [water (formic acid)-acetonitrile]; B %: 32%-62%, 9 min) and lyophilized to afford 1-(methyl(phenyl)carbamoyl)azetidin-3-yl (1-(4-(2,6-dioxopiperidin-3-yl)-3,5-difluorophenyl)azetidin-3-yl) carbamate (36.34 mg, 65.44 umol, 41% yield, 95% purity) as an off-white solid.

¹H NMR (400 MHz, DMSO-d₆) δ=10.85 (s, 1H), 8.03 (br d, J=7.3 Hz, 1H), 7.44-7.34 (m, 2H), 7.29-7.20 (m, 3H), 6.13 (br d, J=11.0 Hz, 2H), 4.84-4.72 (m, 1H), 4.41-4.29 (m, 1H), 4.09-3.98 (m, 3H), 3.77-3.68 (m, 2H), 3.59 (br t, J=6.8 Hz, 2H), 3.31-3.30 (m, 2H), 3.13 (s, 3H), 2.83-2.72 (m, 1H), 2.42 (br s, 1H), 2.12-2.03 (m, 1H), 1.97-1.90 (m, 1H).

¹H NMR (400 MHz, DMSO-d₆+D₂O) δ=10.86 (s, 1H), 8.04 (br d, J=7.4 Hz, 1H), 7.44-7.36 (m, 2H), 7.31-7.20 (m, 3H), 6.19-6.08 (m, 2H), 4.83-4.74 (m, 1H), 4.40-4.29 (m, 1H), 4.09-3.99 (m, 3H), 3.79-3.70 (m, 2H), 3.62-3.57 (m, 2H), 3.33 (br dd, J=3.4, 9.8 Hz, 2H), 3.13 (s, 3H), 2.81-2.72 (m, 1H), 2.45 (br s, 1H), 2.10-2.01 (m, 1H), 1.98-1.89 (m, 1H). MS (ESI) m/z 528.5 [M+H]⁺

### Example 18. Synthesis of Compound 18

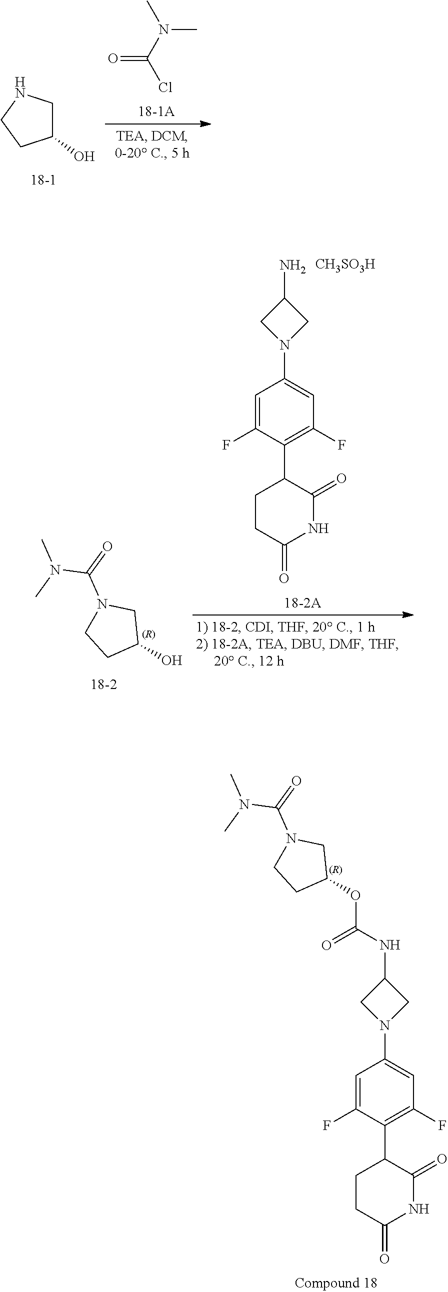

### Step 1. Procedure for Compound 2—(R)-3-hydroxy-N,N-dimethylpyrrolidine-1-carboxamide

To a solution of (R)-pyrrolidin-3-ol (500 mg, 5.74 mmol, 476 uL, 1.00 eq) in dichloromethane (5.00 mL) were added triethylamine (1.22 g, 12.1 mmol, 1.68 mL, 2.10 eq) and dimethylcarbamic chloride (679 mg, 6.31 mmol, 580 uL, 1.10 eq) at 0° C. The reaction mixture was stirred at 20° C. for 5 h. The reaction mixture was filtered and the filtrate was concentrated under reduced pressure to afford (R)-3-hydroxy-N,N-dimethylpyrrolidine-1-carboxamide (900 mg, 5.69 mmol, 99% yield) as a white solid.

¹H NMR (400 MHz, CDCl3) δ=4.40-4.35 (m, 1H), 3.60-3.47 (m, 3H), 3.36-3.31 (m, 1H), 3.28-325 (dd, J=1.9, 11.4 Hz, 1H), 2.79 (s, 6H), 1.90-1.85 (m, 2H).

### Step 2. Procedure for (R)-1-(dimethylcarbamoyl)pyrrolidin-3-yl (1-(4-(2,6-dioxopiperidin-3-yl)-3,5-difluorophenyl)azetidin-3-yl)carbamate

To a solution of (R)-3-hydroxy-N,N-dimethylpyrrolidine-1-carboxamide (60.0 mg, 379 umol, 476 uL, 1.00 eq) in tetrahydrofuran (1.00 mL) was added di(1H-imidazol-1-yl)methanone (123 mg, 759 umol, 2.00 eq). The reaction mixture was stirred at 20° C. for 1 h. The resulting solution was added to a solution of 3-(4-(3-aminoazetidin-1-yl)-2,6-difluorophenyl)piperidine-2,6-dione (147 mg, 377 umol, 1.00 eq, mesylate), triethylamine (38.1 mg, 377 umol, 52.4 uL, 1.00 eq) and 2,3,4,6,7,8,9,10-octahydropyrimido[1,2-a]azepine (57.3 mg, 377 umol, 56.8 uL, 1.00 eq) in tetrahydrofuran (1.00 mL) and dimethyformamide (1.00 mL). The reaction mixture was stirred at 20° C. for 12 h. The reaction mixture was filtered. The filtrate was purified by Prep-HPLC (column: Welch Ultimate C18 150*25 mm*5 um; mobile phase: [water (formic acid)-acetonitrile]; B %: 20%-50%, 10 min) and lyophilized to afford (R)-1-(dimethylcarbamoyl)pyrrolidin-3-yl-(1-(4-(2,6-dioxopiperidin-3-yl)-3,5-difluorophenyl)azetidin-3-yl)carbamate (17.97 mg, 37.10 umol, 10% yield, 99% purity) as a white solid.

¹H NMR (400 MHz, DMSO-d₆) δ=10.86 (s, 1H), 7.97 (br d, J=7.3 Hz, 1H), 6.14 (br d, J=11.0 Hz, 2H), 5.08 (br s, 1H), 4.51-4.34 (m, 1H), 4.13-4.00 (m, 3H), 3.69-3.58 (m, 3H), 3.49-3.37 (m, 2H), 3.22 (br d, J=11.9 Hz, 1H), 2.82-2.76 (m, 1H), 2.73 (s, 6H), 2.18-1.85 (m, 5H). MS (ESI) m/z. 480.3 [M+H]⁺

### Example 19. Synthesis of Compound 19

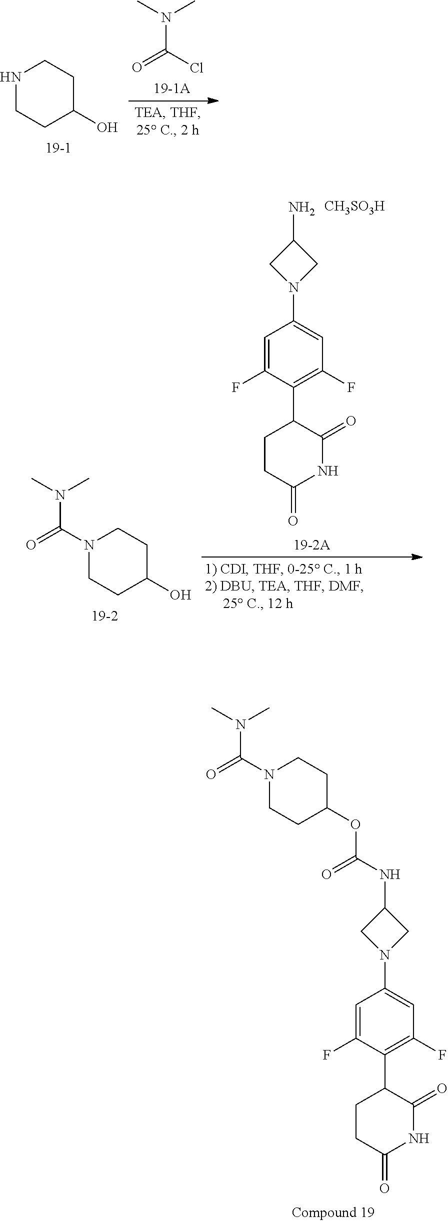

### Step 1. Procedure for Compound 2—4-hydroxy-N,N-dimethylpiperidine-1-carboxamide

To a solution of piperidin-4-ol (200 mg, 1.98 mmol, 1.00 eq) in tetrahydrofuran (4.00 mL) were added dimethylcarbamic chloride (425 mg, 3.95 mmol, 363 uL, 2.00 eq) and triethylamine (500 mg, 4.94 mmol, 688 uL, 2.50 eq). The mixture was stirred at 25° C. for 2 h. The mixture was concentrated under reduced pressure to give a residue. The residue was purified by column chromatography (SiO₂, petroleum ether/ethyl acetate=10/1 to 0/1) to afford 4-hydroxy-N,N-dimethylpiperidine-1-carboxamide (250 mg, 1.45 mmol, 73% yield) as colorless oil. ¹H NMR (400 MHz, CDCl₃-d) δ=3.89-3.79 (m, 1H), 3.60-3.51 (m, 2H), 2.96-2.89 (m, 2H), 2.82 (s, 6H), 1.94-1.86 (m, 2H), 1.58-1.47 (m, 2H).

### Step 2. Procedure for 1-(dimethylcarbamoyl)piperidin-4-yl (1-(4-(2,6-dioxopiperidin-3-yl)-3,5-difluorophenyl)azetidin-3-yl)carbamate

To a solution of 4-hydroxy-N,N-dimethylpiperidine-1-carboxamide (100 mg, 581 umol, 1.00 eq) in tetrahydrofuran (1.00 mL) was added di(1H-imidazol-1-yl)methanone (113 mg, 697 umol, 1.20 eq) at 0° C. The mixture was stirred at 25° C. for 2 h. The resulting solution was added to a mixture of 3-(4-(3-aminoazetidin-1-yl)-2,6-difluorophenyl)piperidine-2,6-dione (166 mg, 425 umol, 0.754 eq, mesylate), 2,3,4,6,7,8,9,10-octahydropyrimido[1,2-a]azepine (85.8 mg, 563 umol, 84.9 uL, 1.00 eq), triethylamine (57.0 mg, 563 umol, 78.4 uL, 1.00 eq) in tetrahydrofuran (1.00 mL) and dimethylformamide (1.00 mL). The mixture was stirred at 25° C. for 12 h. The mixture was concentrated under reduced pressure to give a residue. The residue was purified by reverse phase chromatography (C18, 80 g; condition: water/acetonitrile=100:0 to 0:100, 0.1% formic acid) and lyophilized to afford 1-(dimethylcarbamoyl)piperidin-4-yl (1-(4-(2,6-dioxopiperidin-3-yl)-3,5-difluorophenyl)azetidin-3-yl)carbamate (38.47 mg, 77.17 umol, 13% yield, 99% purity) as an white solid.

¹H NMR (400 MHz, DMSO-d₆) δ=10.85 (s, 1H), 7.86 (br d, J=7.5 Hz, 1H), 6.14 (d, J=11.0 Hz, 2H), 4.75-4.61 (m, 1H), 4.49-4.36 (m, 1H), 4.09 (t, J=7.7 Hz, 2H), 4.06-4.00 (m, 1H), 3.63 (t, J=6.8 Hz, 2H), 3.41-3.33 (m, 2H), 2.98-2.87 (m, 2H), 2.83-2.76 (m, 1H), 2.72 (s, 6H), 2.54 (br s, 1H), 2.13-2.01 (m, 1H), 1.98-1.89 (m, 1H), 1.83 (br d, J=9.0 Hz, 2H), 1.54-1.42 (m, 2H). MS (ESI) m/z 494.4 [M+H]⁺

### Example 20. Synthesis of Compound 20

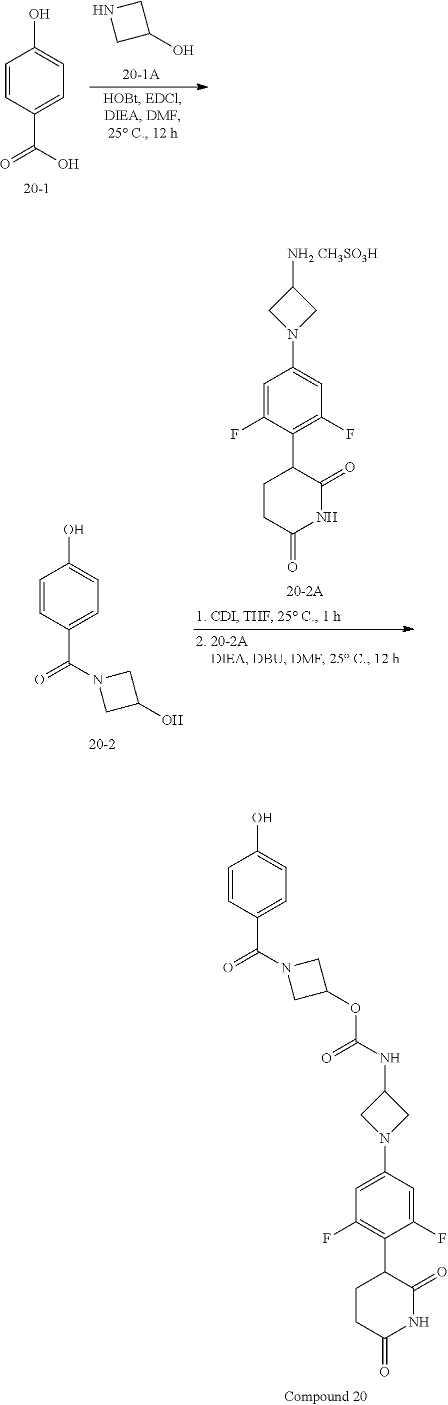

### Step 1. Procedure for Preparation of Compound 2—4-(3-hydroxyazetidine-1-carbonyl)benzonitrile

To a solution of 4-cyanobenzoic acid (100 mg, 679 umol, 1.00 eq) and azetidin-3-ol (89.3 mg, 815 umol, 1.20 eq, hydrochloride) in dimethylformamide (5.00 mL) was added 1H-benzo[d][1,2,3]triazol-1-ol (64.2 mg, 475 umol, 0.700 eq), N,N-diisopropylethylamine (87.8 mg, 679 umol, 118 uL, 1.00 eq) and 1-(3-dimethylaminopropyl)-3-ethylcarbodiimide hydrochloride (143 mg, 747 umol, 1.10 eq). The reaction was stirred at 25° C. for 12 h. The reaction was diluted with water (100 mL) and extracted with ethyl acetate (3×100 mL). The combined organic layers were washed with brine (100 mL), dried over sodium sulfate, filtered and concentrated under reduced pressure to give a residue. The residue was purified by column chromatography (SiO₂, petroleum ether/ethyl acetate=1/1 to 0/1) to afford 4-(3-hydroxyazetidine-1-carbonyl)benzonitrile (110 mg, 543 umol, 80% yield) as a white solid.

¹H NMR (400 MHz, DMSO-d₆) δ=7.93 (br d, J=8.4 Hz, 2H), 7.78 (br d, J=8.4 Hz, 2H), 5.80 (br d, J=6.0 Hz, 1H), 4.55-4.47 (m, 1H), 4.45-4.38 (m, 1H), 4.32-4.22 (m, 1H), 4.03 (br dd, J=3.6, 8.9 Hz, 1H), 3.80 (br dd, J=4.0, 10.3 Hz, 1H).

### Step 2. Procedure for Preparation of 1-(4-cyanobenzoyl)azetidin-3-yl (1-(4-(2,6-dioxopiperidin-3-yl)-3,5-difluorophenyl)azetidin-3-yl)carbamate

To a solution of 4-(3-hydroxyazetidine-1-carbonyl)benzonitrile (110 mg, 543.99 umol, 1.00 eq) in tetrahydrofuran (2.00 mL) was added di(1H-imidazol-1-yl)methanone (88.21 mg, 543.99 umol, 1.00 eq). The mixture was stirred at 25° C. for 1 h. The resulting solution was added to a solution 3-(4-(3-aminoazetidin-1-yl)-2,6-difluorophenyl)piperidine-2,6-dione (157 mg, 401 umol, 7.43^{e-1} eq, mesylate), 2,3,4,6,7,8,9,10-octahydropyrimido[1,2-a]azepine (82.2 mg, 540 umol, 81.4 uL, 1.00 eq) and N,N-diisopropylethylamine (69.7 mg, 540 umol, 94.0 uL, 1.00 eq) in dimethylformamide (1.00 mL). The reaction was stirred at 25° C. for 12 h. The reaction mixture was concentrated under reduced pressure to give a residue. The residue was purified by Prep-HPLC (column: Phenomenex luna C18 150*25 mm*10 um; mobile phase: [water (formic acid)-acetonitrile]; B %: 26%-56%, 58 min) to afford 1-(4-cyanobenzoyl)azetidin-3-yl (1-(4-(2,6-dioxopiperidin-3-yl)-3,5-difluorophenyl)azetidin-3-yl)carbamate (40.57 mg, 75.95 umol, 14% yield, 98% purity) as a white solid.

¹H NMR (400 MHz, DMSO-d₆) δ=10.86 (s, 1H), 8.16 (br d, J=7.2 Hz, 1H), 7.94 (d, J=8.0 Hz, 2H), 7.81 (d, J=8.0 Hz, 2H), 6.16 (br d, J=11.2 Hz, 2H), 5.15-5.07 (m, 1H), 4.59-4.53 (m, 1H), 4.45-4.35 (m, 2H), 4.26 (br d, J=8.4 Hz, 1H), 4.10 (br t, J=7.6 Hz, 2H), 4.06-4.01 (m, 1H), 3.97 (br d, J=10.0 Hz, 1H), 3.66 (br t, J=6.8 Hz, 2H), 2.76 (br dd, J=5.2, 12.9 Hz, 1H), 2.48 (br s, 1H), 2.08 (br dd, J=3.6, 12.9 Hz, 1H), 1.98-1.89 (m, 1H). MS (ESI) m/z 524.1 [M+H]⁺

### Example 21. Synthesis of Compound 21

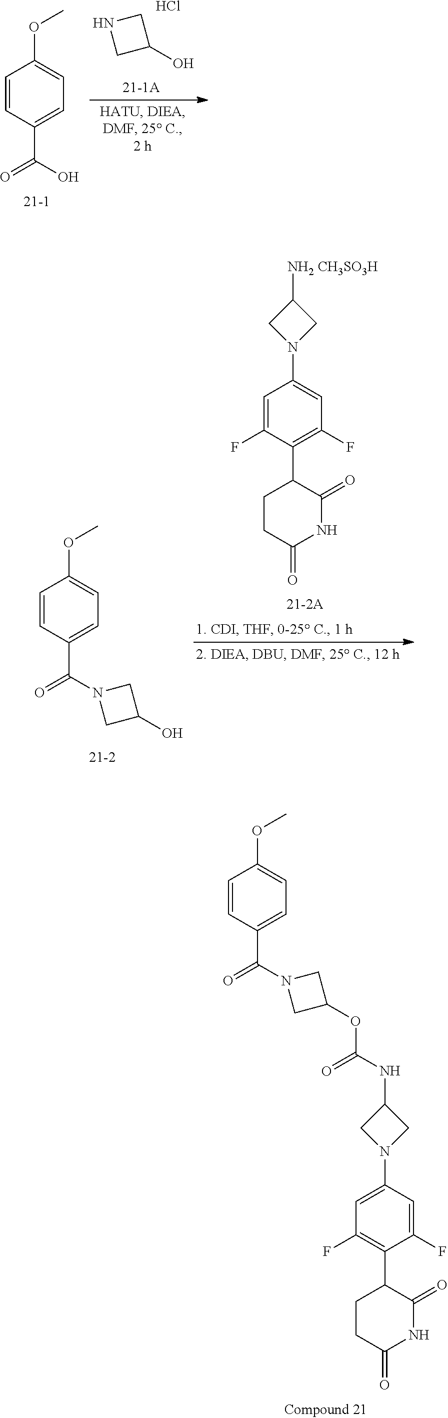

### Step 1. Procedure for Preparation of Compound 2—(3-hydroxyazetidin-1-yl)(4-methoxyphenyl)methanone

To a solution of 4-methoxybenzoic acid (500 mg, 3.29 mmol, 1.00 eq) and azetidin-3-ol (481 mg, 4.39 mmol, 1.33 eq, hydrochloride) in dimethylformamide (5 mL) were added N,N-diisopropylethylamine (1.28 g, 9.87 mmol, 1.72 mL, 3.00 eq) and 2-(3H-[1,2,3]triazolo[4,5-b]pyridin-3-yl)-1,1,3,3-tetramethyluronium hexafluorophosphate(V) (1.88 g, 4.94 mmol, 1.50 eq), the mixture was stirred at 25° C. for 2 h. The reaction mixture was diluted with water (30 mL) and extracted with ethyl acetate (3×30 mL). The combined organic phase was washed with brine (2×20 mL), dried over sodium sulfate, filtered and concentrated under reduced pressure to give a residue. The residue was purified by column chromatography (SiO₂, petroleum ether/ethyl acetate=1/1 to 0/1) to afford (3-hydroxyazetidin-1-yl)(4-methoxyphenyl)methanone (1.00 g, crude) as yellow oil.

¹H NMR (400 MHz, DMSO-d₆) δ=7.60 (d, J=8.8 Hz, 2H), 7.02-6.95 (m, 2H), 5.72 (br s, 1H), 4.48 (br s, 2H), 4.23 (br s, 1H), 4.07-3.96 (m, 1H), 3.81 (s, 3H), 3.78 (br d, J=1.0 Hz, 1H).

### Step 2. Procedure for Preparation of 1-(4-methoxybenzoyl)azetidin-3-yl (1-(4-(2,6-dioxopiperidin-3-yl)-3,5-difluorophenyl)azetidin-3-yl)carbamate

To a solution of (3-hydroxyazetidin-1-yl)(4-methoxyphenyl)methanone (100 mg, 483 umol, 1.00 eq) in tetrahydrofuran (1.00 mL) was added di(1H-imidazol-1-yl)methanone (78.3 mg, 483 umol, 1.00 eq) at 0° C., the mixture was stirred at 25° C. for 1 h. The resulting solution was added to a mixture of 3-(4-(3-aminoazetidin-1-yl)-2,6-difluorophenyl)piperidine-2,6-dione (182 mg, 465 umol, 1.00 eq, mesylate), 2,3,4,6,7,8,9,10-octahydropyrimido[1,2-a]azepine (70.7 mg, 465 umol, 70.0 uL, 1.00 eq) and N,N-diisopropylethylamine (60.0 mg, 465 umol, 80.9 uL, 1.00 eq) in dimethylformamide (1.00 mL), the mixture was stirred at 25° C. for 11 h. The mixture was filtered. The filtrate was purified by Prep-HPLC (column: Phenomenex luna C18 150*25 mm*10 um; mobile phase: [water (formic acid)-acetonitrile]; B %: 33%-53%, 58 min) and lyophilized to afford 1-(4-methoxybenzoyl)azetidin-3-yl (1-(4-(2,6-dioxopiperidin-3-yl)-3,5-difluorophenyl)azetidin-3-yl) carbamate (19.31 mg, 37.5 umol, 8% yield, 96% purity) as a white solid.

¹H NMR (400 MHz, DMSO-d₆) δ=10.86 (s, 1H), 8.15 (br d, J=7.2 Hz, 1H), 7.62 (d, J=8.8 Hz, 2H), 6.99 (d, J=8.8 Hz, 2H), 6.16 (d, J=11.0 Hz, 2H), 5.18-5.07 (m, 1H), 4.68-4.51 (m, 1H), 4.47-4.39 (m, 1H), 4.37-4.28 (m, 1H), 4.23 (dt, J=1.7, 3.2 Hz, 1H), 4.10 (br t, J=7.6 Hz, 2H), 4.04 (br dd, J=5.0, 12.6 Hz, 1H), 3.98-3.84 (m, 1H), 3.81 (s, 3H), 3.66 (br t, J=6.8 Hz, 2H), 2.85-2.72 (m, 1H), 2.48-2.45 (m, 1H), 2.08 (dq, J=4.2, 13.0 Hz, 1H), 2.00-1.89 (m, 1H). MS (ESI) m/z 529.2 [M+H]⁺

### Example 22. Synthesis of Compound 22

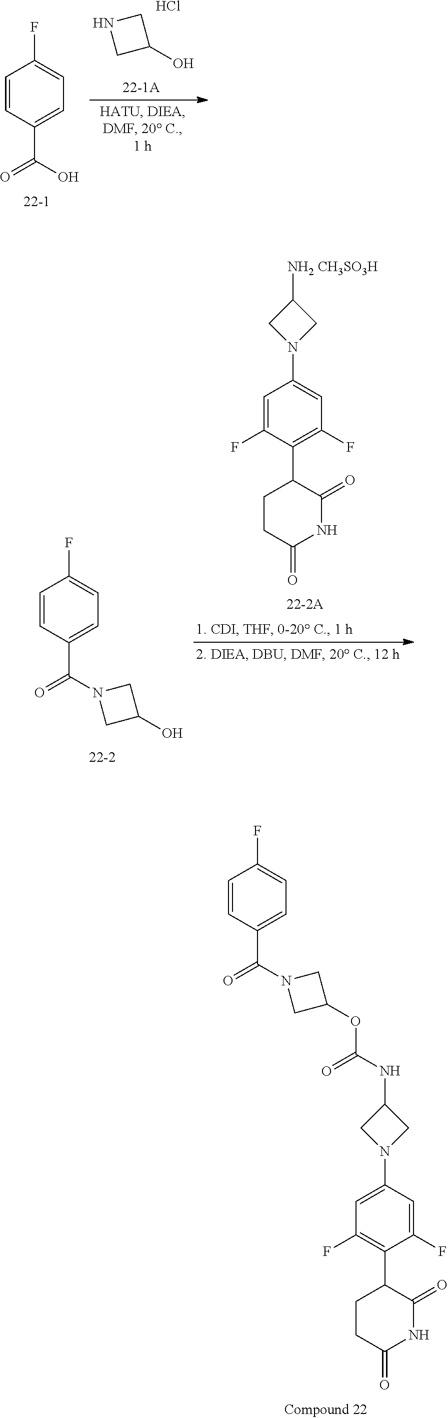

### Step 1. Procedure for Compound 2—(4-fluorophenyl)(3-hydroxyazetidin-1-yl)methanone

To a solution of 4-fluorobenzoic acid (200 mg, 1.43 mmol, 1.00 eq) in N,N-dimethyl formamide (2.00 mL) were added azetidin-3-ol (312 mg, 2.85 mmol, 2.00 eq, hydrochloride), 2-(3H-[1,2,3]triazolo[4,5-b]pyridin-3-yl)-1,1,3,3-tetramethyluronium hexafluorophosphate(V) (814 mg, 2.14 mmol, 1.50 eq) and diisopropylethylamine (553 mg, 4.28 mmol, 745 uL, 3.00 eq). The reaction mixture was stirred at 20° C. for 1 h. The reaction mixture was extracted with ethyl acetate (3×10 mL). The combined organic layers were washed with water (10 mL), dried over anhydrous sodium sulfate, filtered and concentrated under reduced pressure to give a residue. The residue was purified by column chromatography (SiO₂, petroleum ether/ethyl acetate=1/0 to 0/1) to afford (4-fluorophenyl)-(3-hydroxyazetidin-1-yl)methanone (270 mg, 1.38 mmol, 96% yield) as a yellow solid.

¹H NMR (400 MHz, DMSO-d₆) δ=7.72-7.66 (m, 2H), 7.30-7.23 (m, 2H), 5.74 (br d, J=4.8 Hz, 1H), 4.53-4.40 (m, 2H), 4.29-4.19 (m, 1H), 4.03 (q, J=7.1 Hz, 1H), 3.78 (br d, J=7.6 Hz, 1H).

### Step 2. Procedure for 1-(4-fluorobenzoyl)azetidin-3-yl (1-(4-(2,6-dioxopiperidin-3-yl)-3,5-difluorophenyl)azetidin-3-yl)carbamate

To a solution of (4-fluorophenyl)(3-hydroxyazetidin-1-yl)methanone (100 mg, 512 umol, 1.00 eq) in tetrahydrofuran (1.00 mL) was added di(1H-imidazol-1-yl)methanone (83.1 mg, 512 umol, 1.00 eq) at 0° C. The reaction mixture was stirred at 20° C. for 1 h. The resulting solution was added to a mixture of 3-(4-(3-aminoazetidin-1-yl)-2,6-difluorophenyl)piperidine-2,6-dione (196 mg, 501 umol, 1.00 eq, mesylate), 2,3,4,6,7,8,9,10-octahydropyrimido[1,2-a]azepine (76.3 mg, 501 umol, 75.5 uL, 1.00 eq) and N,N-diisopropylethylamine (64.7 mg, 501 umol, 87.3 uL, 1.00 eq) in N,N-dimethyl formamide (1.00 mL). The reaction mixture was stirred at 20° C. for 12 h. The reaction mixture was concentrated under reduced pressure to give a residue. The residue was dissolved in N,N-dimethyl formamide (0.5 mL) and then filtered. The filtrate was purified by Prep-HPLC (column: Phenomenex luna C18 150*25 mm*10 um; mobile phase: [water (formic acid)-acetonitrile]; B %: 30%-60%, 9 min) and lyophilized to afford 1-(4-fluorobenzoyl)azetidin-3-yl (1-(4-(2,6-dioxopiperidin-3-yl)-3,5-difluorophenyl)azetidin-3-yl)carbamate (10.61 mg, 19.31 umol, 3% yield, 94% purity) as an off-white solid.

¹H NMR (400 MHz, DMSO-d₆) δ=10.86 (s, 1H), 8.15 (d, J=7.4 Hz, 1H), 7.72 (dd, J=5.6, 8.8 Hz, 2H), 7.32-7.24 (m, 2H), 6.15 (d, J=11.0 Hz, 2H), 5.15-5.07 (m, 1H), 4.62-4.51 (m, 1H), 4.47-4.39 (m, 1H), 4.39-4.31 (m, 1H), 4.29-4.19 (m, 1H), 4.13-4.07 (m, 2H), 4.06-4.00 (m, 1H), 3.98-3.88 (m, 1H), 3.65 (br t, J=6.6 Hz, 2H), 2.84-2.72 (m, 1H), 2.48-2.44 (m, 1H), 2.12-2.02 (m, 1H), 1.98-1.90 (m, 1H). MS (ESI) m/z. 517.0 [M+H]⁺

### Example 23. Synthesis of Compound 23

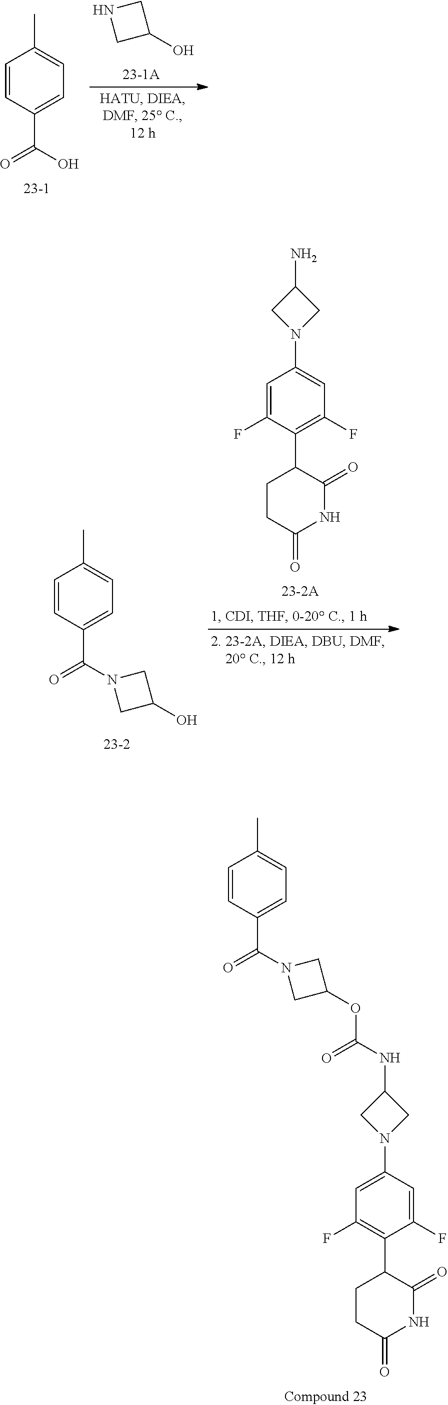

### Step 1. Procedure for Preparation of Compound 2—(3-hydroxyazetidin-1-yl)(p-tolyl)methanone

To a solution of 4-methylbenzoic acid (500 mg, 3.67 mmol, 1.00 eq) in dimethylformamide (5.00 mL) were added 2-(3H-[1,2,3]triazolo[4,5-b]pyridin-3-yl)-1,1,3,3-tetramethyluronium hexafluorophosphate(V) (1.68 g, 4.41 mmol, 1.20 eq), N,N-diisopropylethylamine (1.42 g, 11.0 mmol, 1.92 mL, 3.00 eq) and azetidin-3-ol (322 mg, 4.41 mmol, 1.20 eq, hydrochloride). The mixture was stirred at 25° C. for 12 h. The reaction was filtered. The filtrate was purified by reverse phase chromatography (C18, 40 g; condition: water/acetonitrile=100:0 to 0:100, 0.1% formic acid) and lyophilized to afford (3-hydroxyazetidin-1-yl)(p-tolyl)methanone (350 mg, 1.76 mmol, 48% yield, 96% purity) as a yellow solid.

¹H NMR (400 MHz, DMSO-d₆) δ=7.50 (d, J=8.0 Hz, 2H), 7.25 (d, J=7.9 Hz, 2H), 5.74 (br d, J=5.9 Hz, 1H), 4.52-4.45 (m, 1H), 4.43 (br d, J=9.5 Hz, 1H), 4.23 (br d, J=8.0 Hz, 1H), 4.00 (br d, J=4.0 Hz, 1H), 3.76 (br d, J=6.9 Hz, 1H), 2.34 (s, 3H).

### Step 2. Procedure for Preparation of 1-(4-methylbenzoyl)azetidin-3-yl (1-(4-(2,6-dioxopiperidin-3-yl)-3,5-difluorophenyl)azetidin-3-yl)carbamate

To a solution of (3-hydroxyazetidin-1-yl)(p-tolyl)methanone (100 mg, 523 umol, 1.00 eq) in tetrahydrofuran (2.00 mL) was added di(1H-imidazol-1-yl)methanone (84.8 mg, 523 umol, 1.00 eq) at 0° C., then the mixture was stirred at 20° C. for 1 h. The resulting solution was added to a mixture of 3-(4-(3-aminoazetidin-1-yl)-2,6-difluorophenyl)piperidine-2,6-dione (154 mg, 522 umol, 1.00 eq, mesylate), 2,3,4,6,7,8,9,10-octahydropyrimido[1,2-a]azepine (79.5 mg, 522 umol, 78.7 uL, 1.00 eq) and N,N-diisopropylethylamine (67.5 mg, 522 umol, 91.0 uL, 1.00 eq) in dimethylformamide (2.00 mL). The mixture was stirred at 20° C. for 12 h. The reaction was filtered. The filtrate was purified by Prep-HPLC (column: Phenomenex luna C18 150*25 mm*10 um; mobile phase: [water (formic acid)-acetonitrile]; B %: 30%-60%, 10 min) and lyophilized to afford 1-(4-methylbenzoyl)azetidin-3-yl (1-(4-(2,6-dioxopiperidin-3-yl)-3,5-difluorophenyl)azetidin-3-yl)carbamate (45.25 mg, 88.34 umol, 17% yield, 97% purity) as a white solid.

¹H NMR (400 MHz, DMSO-d₆) δ=10.86 (s, 1H), 8.14 (br d, J=7.4 Hz, 1H), 7.54 (d, J=8.0 Hz, 2H), 7.26 (d, J=7.9 Hz, 2H), 6.15 (d, J=11.0 Hz, 2H), 5.20-4.99 (m, 1H), 4.56 (br s, 1H), 4.48-4.39 (m, 1H), 4.38-4.28 (m, 1H), 4.28-4.15 (m, 1H), 4.10 (br t, J=7.6 Hz, 2H), 4.03 (br dd, J=5.0, 12.6 Hz, 1H), 3.92 (br d, J=7.4 Hz, 1H), 3.65 (br t, J=6.6 Hz, 2H), 2.85-2.70 (m, 1H), 2.47 (br s, 1H), 2.35 (s, 3H), 2.14-2.00 (m, 1H), 1.98-1.88 (m, 1H). MS (ESI) m/z 513.1 [M+H]⁺

### Example 24. Synthesis of Compound 24

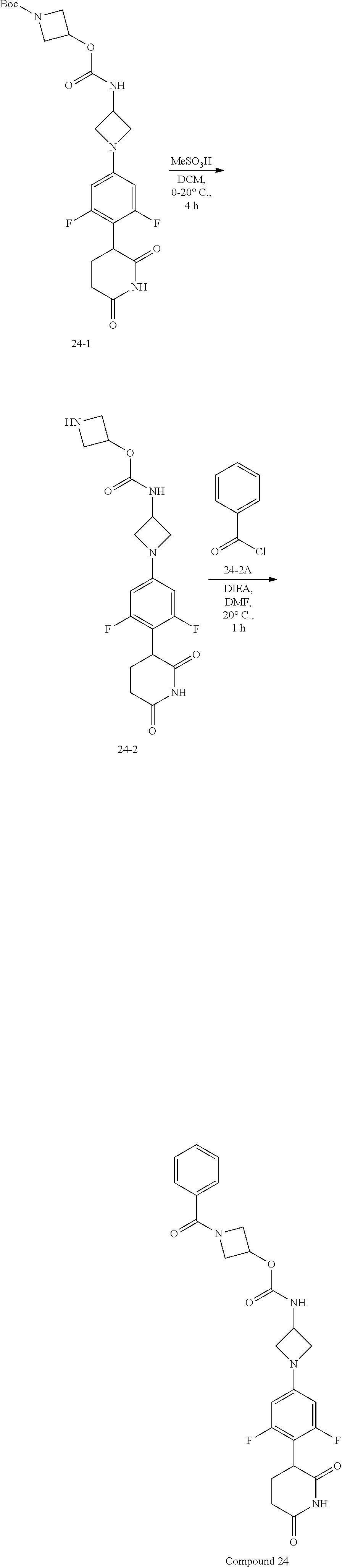 [SMILES: "MeSO3H" ; CC(C)(C)OC(=O)N1CC(OC(=O)NC2CN(c3cc(F)c(C4CCC(=O)NC4=O)c(F)c3)C2)C1 ; O=C1CCC(c2c(F)cc(N3CC(NC(=O)OC4CNC4)C3)cc2F)C(=O)N1 ; O=C(Cl)c1ccccc1 ; O=C1CCC(c2c(F)cc(N3CC(NC(=O)OC4CN(C(=O)c5ccccc5)C4)C3)cc2F)C(=O)N1]

### Step 1. Procedure for Preparation of Compound 2—azetidin-3-yl (1-(4-(2,6-dioxopiperidin-3-yl)-3,5-difluorophenyl)azetidin-3-yl)carbamate

To a solution of tert-butyl 3-(((1-(4-(2,6-dioxopiperidin-3-yl)-3,5-difluorophenyl)azetidin-3-yl)carbamoyl) oxy)azetidine-1-carboxylate (100 mg, 202 umol, 1.00 eq) in dichloromethane (1.00 mL) was added methanesulfonic acid (58.3 mg, 607 umol, 43.2 uL, 3.00 eq) at 0° C. The mixture was stirred at 0° C. for 0.5 h and then warmed to 20° C. The reaction mixture was stirred at 20° C. for 3.5 h. The reaction mixture was concentrated under reduced pressure to afford azetidin-3-yl (1-(4-(2,6-dioxopiperidin-3-yl)-3,5-difluorophenyl)azetidin-3-yl)carbamate (99.0 mg, crude, mesylate) as yellow oil. MS (ESI) m/z 395.1 [M+H]⁺

### Step 2. Procedure for Preparation of 1-benzoylazetidin-3-yl (1-(4-(2,6-dioxopiperidin-3-yl)-3,5-difluorophenyl)azetidin-3-yl)carbamate

To a solution of azetidin-3-yl (1-(4-(2,6-dioxopiperidin-3-yl)-3,5-difluorophenyl)azetidin-3-yl)carbamate (99.0 mg, 202 umol, 1.00 eq, mesylate) in dimethylformamide (1.00 mL) were added N,N-diisopropylethylamine (78.3 mg, 606 umol, 106 uL, 3.00 eq) and benzoyl chloride (42.6 mg, 303 umol, 35.2 uL, 1.50 eq). The reaction mixture was stirred at 20° C. for 1 h. The reaction mixture was filtered. The filtrate was purified by reverse phase chromatography (C18, 40 g; condition: water/acetonitrile=100:0 to 52:48, 0.1% formic acid) and lyophilized to afford 1-benzoylazetidin-3-yl (1-(4-(2,6-dioxopiperidin-3-yl)-3,5-difluorophenyl)azetidin-3-yl)carbamate (33.31 mg, 65.49 umol, 32% yield, 98% purity) as a white solid.

¹H NMR (400 MHz, DMSO-d₆) δ=10.85 (s, 1H), 8.15 (br d, J=7.3 Hz, 1H), 7.64 (br d, J=7.0 Hz, 2H), 7.56-7.50 (m, 1H), 7.49-7.41 (m, 2H), 6.16 (s, 1H), 6.14 (s, 1H), 5.18-5.06 (m, 1H), 4.65-4.51 (m, 1H), 4.48-4.32 (m, 2H), 4.30-4.17 (m, 1H), 4.10 (br t, J=7.5 Hz, 2H), 4.03 (br dd, J=5.0, 12.8 Hz, 1H), 3.98-3.89 (m, 1H), 3.65 (br t, J=6.6 Hz, 2H), 2.82-2.73 (m, 1H), 2.48-2.46 (m, 1H), 2.12-2.03 (m, 1H), 1.99-1.89 (m, 1H). MS (ESI) m/z 499.2 [M+H]⁺

### Example 25. Synthesis of Compound 25

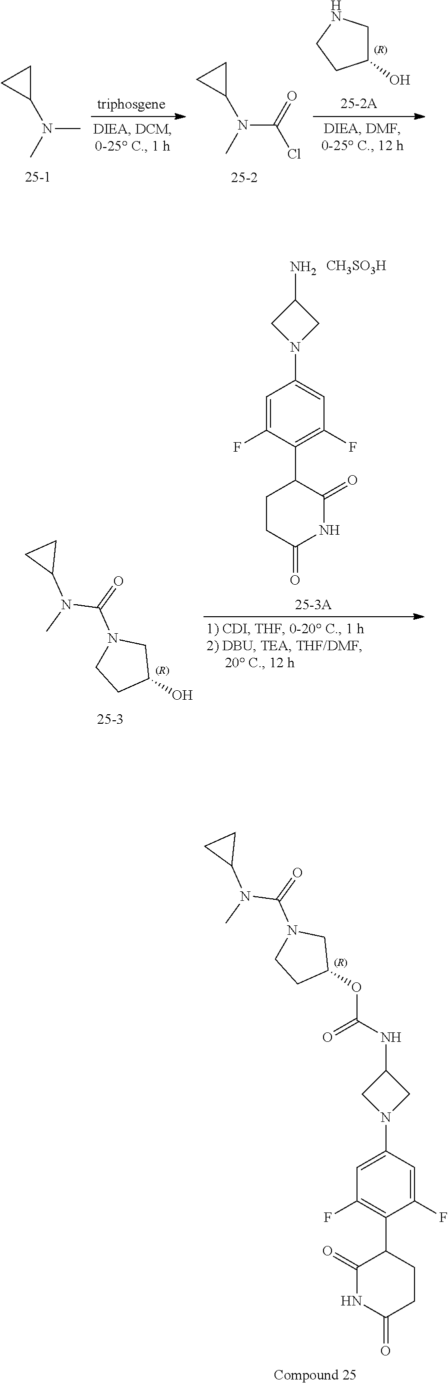

### Step 1. Procedure for Preparation of Compound 2—cyclopropyl(methyl)carbamic chloride

To a solution of N-methylcyclopropanamine (500 mg, 7.03 mmol, 1.00 eq) in dichloromethane (5.00 mL) were added bis(trichloromethyl) carbonate (2.09 g, 7.03 mmol, 1.00 eq) and N,N-diisopropylethylamine (1.82 g, 14.1 mmol, 2.45 mL, 2.00 eq) at 0° C. The mixture was stirred at 25° C. for 1 h. The reaction mixture was concentrated under reduced pressure to afford cyclopropyl(methyl)carbamic chloride (930 mg, crude) as a yellow solid.

### Step 2. Procedure for Preparation of Compound 3—(R)—N-cyclopropyl-3-hydroxy-N-methylpyrrolidine-1-carboxamide

To a solution of (R)-pyrrolidin-3-ol (500 mg, 5.74 mmol, 476 uL, 1.00 eq) in dimethylformamide (5.00 mL) were added N,N-diisopropylethylamine (2.23 g, 17.2 mmol, 3.00 mL, 3.00 eq) and cyclopropyl(methyl)carbamic chloride (919 mg, 6.89 mmol, 1.20 eq) at 0° C. The mixture was stirred at 25° C. for 12 h. The reaction mixture was concentrated under reduce pressure to give a residue. The residue was purified by reverse phase chromatography (C18, 40 g; condition: water/acetonitrile=100:0 to 0:100, 0.1% formic acid) to afford (R)—N-cyclopropyl-3-hydroxy-N-methylpyrrolidine-1-carboxamide (370 mg, 2.01 mmol, 35% yield) as a white solid.

¹H NMR (400 MHz, DMSO-d₆) δ=4.84 (br d, J=3.1 Hz, 1H), 4.19 (br s, 1H), 3.48-3.41 (m, 2H), 3.27 (br dd, J=2.0, 3.6 Hz, 1H), 3.11 (br d, J=11.1 Hz, 1H), 2.69 (s, 3H), 2.58 (td, J=3.3, 6.9 Hz, 1H), 1.84-1.74 (m, 1H), 1.73-1.64 (m, 1H), 0.73-0.66 (m, 1H), 0.65-0.58 (m, 1H), 0.53-0.40 (m, 2H).

### Step 3. Procedure for Preparation of (R)-1-(cyclopropyl(methyl)carbamoyl)pyrrolidin-3-yl (1-(4-(2,6-dioxopiperidin-3-yl)-3,5-difluorophenyl)azetidin-3-yl)carbamate

To a solution of (R)—N-cyclopropyl-3-hydroxy-N-methylpyrrolidine-1-carboxamide (50.0 mg, 271 umol, 1.00 eq) in tetrahydrofuran (1.00 mL) was added di(1H-imidazol-1-yl)methanone (88.0 mg, 542 umol, 2.00 eq) at 0° C. The reaction mixture was stirred at 20° C. for 1 h. The resulting solution was added to a solution of 3-(4-(3-aminoazetidin-1-yl)-2,6-difluorophenyl)piperidine-2,6-dione (75.0 mg, 269 umol, 1.00 eq, mesylate), triethylamine (27.3 mg, 270 umol, 37.5 uL, 1.00 eq) and 2,3,4,6,7,8,9,10-octahydropyrimido[1,2-a]azepine (41.0 mg, 270 umol, 40.6 uL, 1.00 eq) in tetrahydrofuran (1.00 mL) and dimethylformamide (1.00 mL). The reaction mixture was stirred at 20° C. for 12 h. The reaction mixture was concentrated under reduced pressure to give a residue. The residue was purified by reverse phase chromatography (C18, 40 g; condition: water/acetonitrile=100:0 to 0:100, 0.1% formic acid) to afford (R)-1-(cyclopropyl(methyl)carbamoyl)pyrrolidin-3-yl (1-(4-(2,6-dioxopiperidin-3-yl)-3,5-difluorophenyl)azetidin-3-yl)carbamate (26.33 mg, 51.56 umol, 19% yield, 99% purity) as a white solid.

¹H NMR (400 MHz, DMSO-d₆) δ=10.87 (s, 1H), 7.97 (br d, J=7.5 Hz, 1H), 6.14 (br d, J=11.0 Hz, 2H), 5.08 (br s, 1H), 4.50-4.33 (m, 1H), 4.09-4.00 (m, 3H), 3.67-3.59 (m, 3H), 3.47-3.35 (m, 2H), 3.29 (br s, 1H), 2.83-2.73 (m, 1H), 2.69 (s, 3H), 2.63-2.59 (m, 1H), 2.14-1.86 (m, 5H), 0.75-0.68 (m, 1H), 0.65-0.59 (m, 1H), 0.55-0.49 (m, 2H). MS (ESI) m/z. 506.2 [M+H]⁺

### Example 26. Synthesis of Compound 26

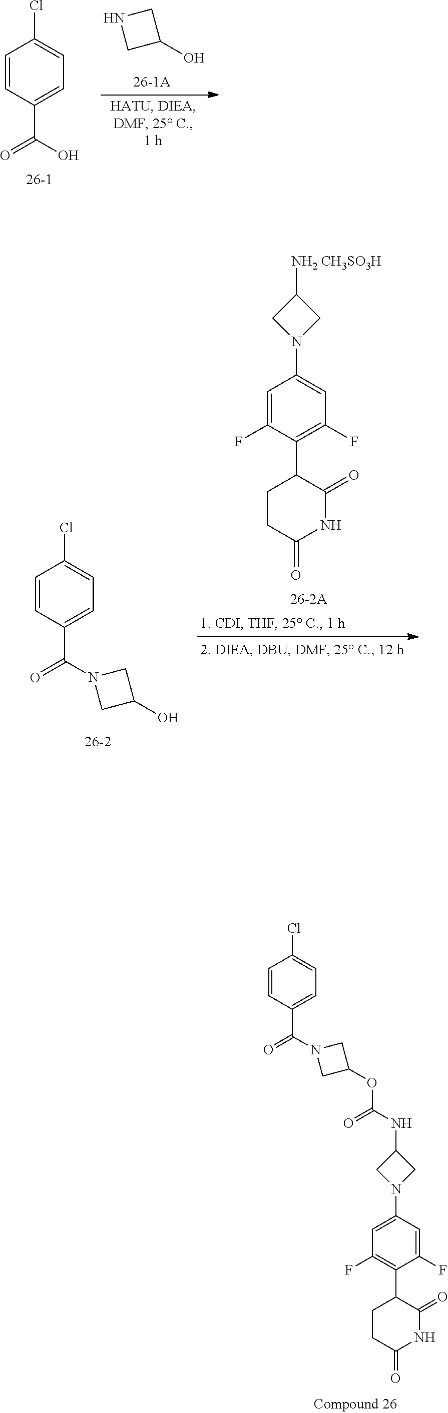

### Step 1. Procedure for Preparation of Compound 2—(4-chlorophenyl)(3-hydroxyazetidin-1-yl)methanone

To a solution of 4-chlorobenzoic acid (500 mg, 3.19 mmol, 1.00 eq), azetidin-3-ol (700 mg, 6.39 mmol, 2.00 eq, hydrochloride), 2-(3H-[1,2,3]triazolo[4,5-b]pyridin-3-yl)-1,1,3,3-tetramethyluronium hexafluorophosphate(V) (1.82 g, 4.79 mmol, 1.50 eq) and N,N-diisopropylethyl amine (1.24 g, 9.58 mmol, 1.67 mL, 3.00 eq) in dimethylformamide (5.00 mL) was stirred at 25° C. for 1 h. The reaction mixture was poured into water (80 mL) and extracted with ethyl acetate (3×30 mL). The combined organic phase was washed with brine (100 mL), dried over anhydrous sodium sulfate, filtered and concentrated in vacuum to give a residue. The residue was purified by column chromatography (SiO₂, petroleum ether/ethyl acetate=1/0 to 0/1) to afford (4-chlorophenyl)(3-hydroxyazetidin-1-yl)methanone (900 mg, crude) as colorless oil.

¹H NMR (400 MHz, DMSO-d₆) δ=7.73-7.62 (m, 2H), 7.58-7.46 (m, 2H), 5.76 (d, J=6.0 Hz, 1H), 4.58-4.42 (m, 2H), 4.30-4.20 (m, 1H), 4.03 (q, J=7.0 Hz, 1H), 3.88-3.70 (m, 1H).

### Step 2. Procedure for Preparation of 1-(4-chlorobenzoyl)azetidin-3-yl (1-(4-(2,6-dioxopiperidin-3-yl)-3,5-difluorophenyl)azetidin-3-yl)carbamate

To a solution of (4-chlorophenyl)-(3-hydroxyazetidin-1-yl)methanone (50.0 mg, 236 umol, 1.00 eq) in tetrahydrofuran (1.00 mL) was added di(1H-imidazol-1-yl)methanone (38.3 mg, 236 umol, 1.00 eq), the mixture was stirred at 25° C. for 1 h. The resulting solution was added to a solution of 3-(4-(3-aminoazetidin-1-yl)-2,6-difluorophenyl)piperidine-2,6-dione (82.3 mg, 228 umol, 1.00 eq, mesylate), 2,3,4,6,7,8,9,10-octahydropyrimido[1,2-a]azepine (34.9 mg, 228 umol, 34.5 uL, 1.00 eq) and N,N-diisopropylethylamine (29.6 mg, 228 umol, 39.9 uL, 1.00 eq) in dimethylformamide (1.00 mL), the mixture was stirred at 25° C. for 12 h. The reaction mixture was concentrated under reduced pressure to give a residue. The residue was purified by Prep-HPLC (column: Phenomenex luna C18 150*25 mm*10 um; mobile phase: [water (formic acid)-acetonitrile]; B %: 34%-64%, 9 min) and lyophilized to afford 1-(4-chlorobenzoyl)azetidin-3-yl (1-(4-(2,6-dioxopiperidin-3-yl)-3,5-difluorophenyl)azetidin-3-yl)carbamate (21.71 mg, 40.31 umol, 17% yield, 99% purity) as a white solid.

¹H NMR (400 MHz, DMSO-d₆) δ=10.86 (s, 1H), 8.15 (br d, J=7.4 Hz, 1H), 7.66 (d, J=8.4 Hz, 2H), 7.51 (d, J=8.5 Hz, 2H), 6.15 (d, J=11.1 Hz, 2H), 5.18-5.03 (m, 1H), 4.64-4.51 (m, 1H), 4.47-4.32 (m, 2H), 4.24 (br d, J=7.0 Hz, 1H), 4.10 (br t, J=7.4 Hz, 2H), 4.03 (br dd, J=5.1, 12.5 Hz, 1H), 3.93 (br d, J=9.6 Hz, 1H), 3.65 (br t, J=6.6 Hz, 2H), 2.84-2.71 (m, 1H), 2.47 (br s, 1H), 2.14-2.02 (m, 1H), 1.99-1.89 (m, 1H). MS (ESI) m/z 533.0 [M+H]⁺

### Example 27. Synthesis of Compound 27

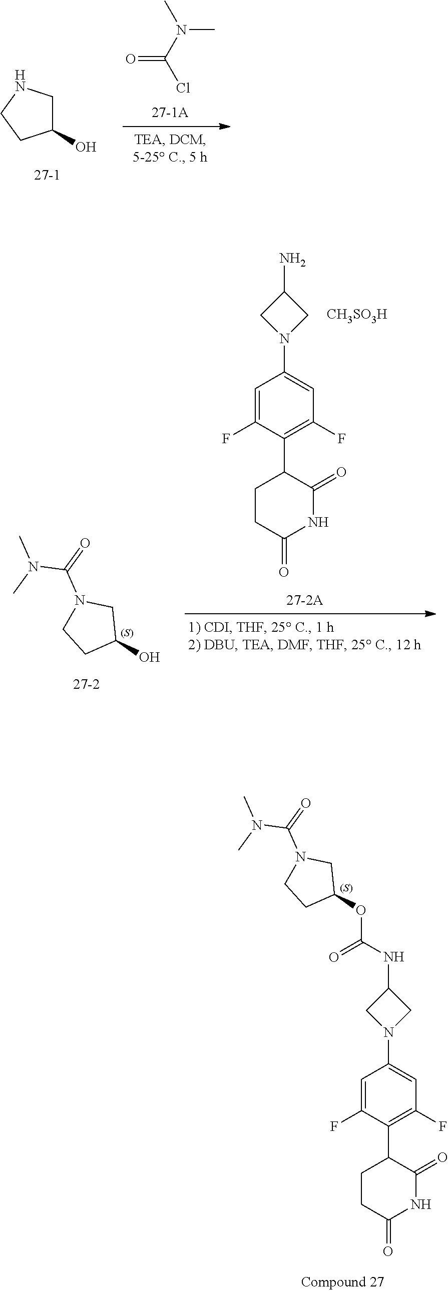

### Step 1. Procedure for Preparation of Compound 2—(S)-3-hydroxy-N,N-dimethylpyrrolidine-1-carboxamide

To a solution of (S)-pyrrolidin-3-ol (300 mg, 3.44 mmol, 278 uL, 1.00 eq) in dichloromethane (5.00 mL) were added triethylamine (732 mg, 7.23 mmol, 1.01 mL, 2.10 eq) and dimethylcarbamic chloride (407 mg, 3.79 mmol, 348 uL, 1.10 eq) at 5° C. The reaction mixture was stirred at 25° C. for 5 h. The reaction mixture was concentrated under reduced pressure to afford (S)-3-hydroxy-N,N-dimethylpyrrolidine-1-carboxamide (250 mg, crude) as a yellow solid.

¹H NMR (400 MHz, DMSO-d₆) δ=4.89-4.82 (m, 1H), 4.25-4.14 (m, 1H), 3.45-3.38 (m, 2H), 3.21 (ddd, J=3.7, 8.0, 10.1 Hz, 1H), 2.99 (s, 1H), 2.71 (s, 6H), 1.84-1.74 (m, 1H), 1.73-1.66 (m, 1H).

### Step 2. Procedure for Preparation of (S)-1-(dimethylcarbamoyl)pyrrolidin-3-yl (1-(4-(2,6-dioxopiperidin-3-yl)-3,5-difluorophenyl)azetidin-3-yl)carbamate

To a solution of (S)-3-hydroxy-N,N-dimethylpyrrolidine-1-carboxamide (130 mg, 822 umol, 1.00 eq) in tetrahydrofuran (2.00 mL) was added di(1H-imidazol-1-yl)methanone (200 mg, 1.23 mmol, 1.50 eq). The reaction mixture was stirred at 25° C. for 1 h. The resulting solution was added to a mixture of 3-(4-(3-aminoazetidin-1-yl)-2,6-difluorophenyl)piperidine-2,6-dione (150 mg, 383 umol, 1.00 eq, mesylate), 2,3,4,6,7,8,9,10-octahydropyrimido[1,2-a]azepine (58.3 mg, 383 umol, 57.8 uL, 1.00 eq), triethylamine (38.8 mg, 383 umol, 53.3 uL, 1.00 eq) in tetrahydrofuran (1.00 mL) and dimethylformamide (1.00 mL). The reaction mixture was stirred at 25° C. for 12 h. The reaction mixture was concentrated under reduced pressure to give a residue. The residue was purified by Prep-HPLC (column: Phenomenex luna C18 150*25 mm*10 um; mobile phase: [water (formic acid)-acetonitrile]; B %: 20%-50%, 10 min) and lyophilized to afford (S)-1-(dimethylcarbamoyl)pyrrolidin-3-yl (1-(4-(2,6-dioxopiperidin-3-yl)-3,5-difluorophenyl)azetidin-3-yl)carbamate (45.43 mg, 93.80 umol, 24% yield, 99% purity) as a white solid.

¹H NMR (400 MHz, DMSO-d₆) δ=10.88 (s, 1H), 7.99 (br d, J=7.1 Hz, 1H), 6.16 (d, J=11.0 Hz, 2H), 5.10 (br s, 1H), 4.50-4.40 (m, 1H), 4.15-4.07 (m, 2H), 4.07-4.01 (m, 1H), 3.69-3.63 (m, 2H), 3.61 (br d, J=4.5 Hz, 1H), 3.44 (dt, J=7.3, 10.0 Hz, 1H), 3.33-3.29 (m, 1H), 3.24 (br d, J=11.9 Hz, 1H), 2.86-2.78 (m, 1H), 2.75 (s, 6H), 2.50 (br s, 1H), 2.16-1.87 (m, 4H). MS (ESI) m/z. 480.3 [M+H]⁺

### Example 28. Synthesis of Compound 28

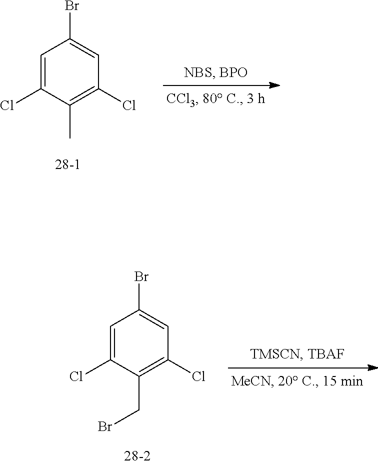

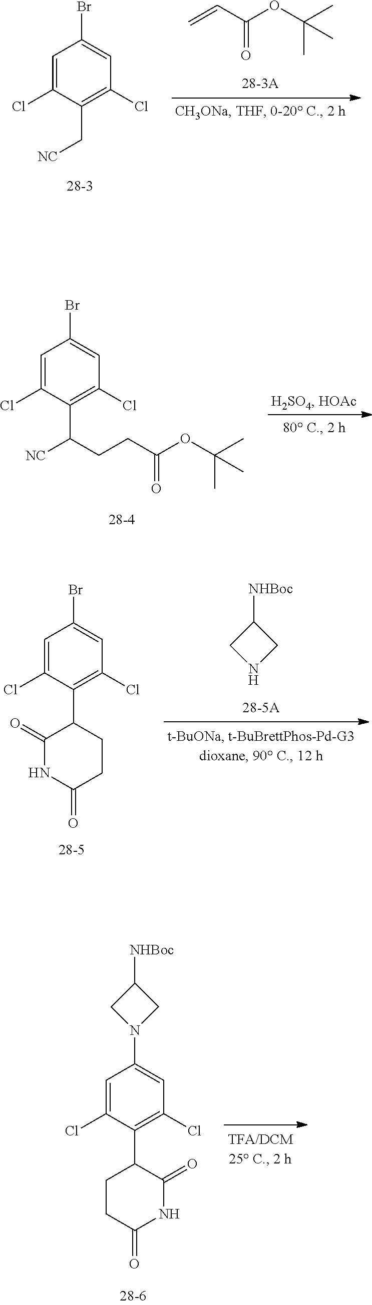

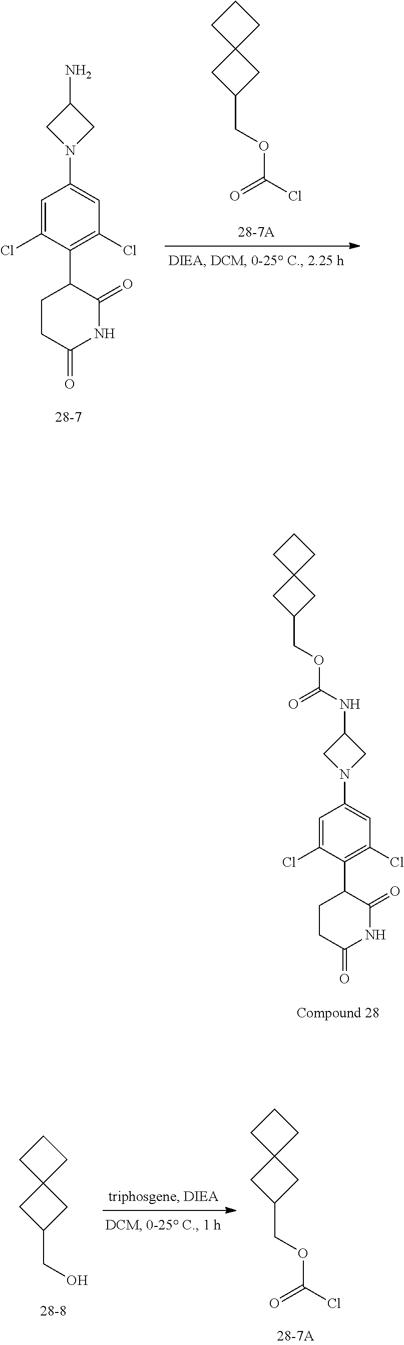 [SMILES: NC1CN(c2cc(Cl)c(C3CCC(=O)NC3=O)c(Cl)c2)C1 ; O=C(Cl)OCC1CC2(CCC2)C1 ; O=C1CCC(c2c(Cl)cc(N3CC(NC(=O)OCC4CC5(CCC5)C4)C3)cc2Cl)C(=O)N1 ; OCC1CC2(CCC2)C1 ; O=C(Cl)OCC1CC2(CCC2)C1]

### Step 1. Procedure for Preparation of Compound 2—5-bromo-2-(bromomethyl)-1,3-dichlorobenzene

A solution of 5-bromo-1,3-dichloro-2-methylbenzene (10.0 g, 41.7 mmol, 1.00 eq) and N-bromosuccinimide (7.42 g, 41.7 mmol, 1.00 eq) in carbon tetrachloride (50.0 mL) was added dibenzoyl peroxide (1.01 g, 4.17 mmol, 0.100 eq) under nitrogen atmosphere. The mixture was stirred 80° C. for 3 h under nitrogen atmosphere. The reaction mixture was cooled to 25° C. Ethyl acetate (40 mL) and water (40 mL) were added and layers were separated. The aqueous phase was extracted with ethyl acetate (2×30 mL). Combined extracts were washed with brine (40 mL), dried over sodium sulfate and filtered. The filtrate was concentrated under reduced pressure to give a residue. The residue was purified by column chromatography (SiO₂, petroleum ether/ethyl acetate=1/0) to afford 5-bromo-2-(bromomethyl)-1,3-dichlorobenzene (16.7 g, crude) as a white solid.

¹H NMR (400 MHz, DMSO-d₆) δ=7.87 (s, 2H), 4.74 (s, 2H).

### Step 2. Procedure for Preparation of Compound 3—2-(4-bromo-2,6-dichlorophenyl)acetonitrile

To a solution of 5-bromo-2-(bromomethyl)-1,3-dichlorobenzene (12.0 g, 37.6 mmol, 1.00 eq) and tetrabutylammonium fluoride (1.00 M, 113 mL, 3.00 eq) in acetonitrile (70.0 mL) was added 2-bromo-5-(bromomethyl)pyridine (11.2 g, 113 mmol, 14.1 mL, 3.00 eq). The mixture was stirred 20° C. for 15 min. The reaction mixture was diluted with ethyl acetate (200 mL) and water (200 mL). The mixture was extracted with dichloromethane (3×200 mL). The combined organic extracts were washed with brine (200 mL), dried over sodium sulfate and filtered. The filtrate was concentrated under reduced pressure to give a residue. The residue was purified by column chromatography (SiO₂, petroleum ether/ethyl acetate=100/1) to afford 2-(4-bromo-2,6-dichloro-phenyl)acetonitrile (9.00 g, 34.0 mmol, 90% yield) as a white solid.

¹H NMR (400 MHz, DMSO-d₆) δ=7.93 (s, 2H), 4.15 (s, 2H).

### Step 3. Procedure for Preparation of Compound 4—tert-butyl 4-(4-bromo-2,6-dichlorophenyl)-4-cyanobutanoate

To mixture of 2-(4-bromo-2,6-dichlorophenyl)acetonitrile (9.00 g, 34.0 mmol, 1.00 eq) and sodium methylate (184 mg, 3.40 mmol, 0.100 eq) in tetrahydrofuran (40.0 mL) was added tert-butyl acrylate (6.53 g, 51.0 mmol, 7.40 mL, 1.50 eq) at 0° C. The mixture was stirred at 20° C. for 2 h. The mixture was concentrated under reduced pressure to afford tert-butyl 4-(4-bromo-2,6-dichlorophenyl)-4-cyanobutanoate (12.0 g, crude) as a white solid. MS (ESI) m/z 393.8 [M+H+2]⁺

### Step 4. Procedure for Preparation of Compound 5—3-(4-bromo-2,6-dichlorophenyl)piperidine-2,6-dione

To mixture of tert-butyl 4-(4-bromo-2,6-dichlorophenyl)-4-cyanobutanoate (11.8 g, 30.0 mmol, 1.00 eq) in acetic acid (20.0 mL) was added sulfuric acid (4.00 mL). The mixture was stirred at 90° C. for 2 h. The reaction mixture was cooled to room temperature and concentrated under reduced pressure to give crude product. The crude product was triturated with water (150 mL) at 25° C. for 10 min and the filtered. The filter cake was concentrated under reduced pressure to afford 3-(4-bromo-2,6-dichlorophenyl)piperidine-2,6-dione (9.50 g, 28.2 mmol, 93% yield) as a white solid.

¹H NMR (400 MHz, DMSO-d₆) δ=11.01 (s, 1H), 7.89-7.83 (m, 1H), 7.80 (d, J=2.0 Hz, 1H), 4.60 (dd, J=5.6, 12.6 Hz, 1H), 2.91-2.80 (m, 1H), 2.58-2.52 (m, 1H), 2.36 (br dd, J=4.2, 13.4 Hz, 1H), 1.94-1.88 (m, 1H).

### Step 5. Procedure for Preparation of Compound 6—tert-butyl (1-(3,5-dichloro-4-(2,6-dioxopiperidin-3-yl)phenyl)azetidin-3-yl)carbamate

To a solution of 3-(4-bromo-2,6-dichlorophenyl)piperidine-2,6-dione (300 mg, 890 umol, 1.00 eq) in dioxane (5.00 mL) were added tert-butyl azetidin-3-ylcarbamate (307 mg, 1.78 mmol, 2.00 eq), sodium tert-butoxide (171 mg, 1.78 mmol, 2.00 eq) was added methanesulfonato[2-(di-tert-butylphosphino)-3,6-dimethoxy-2′,4′,6′-tri-i-propyl-1,1′-biphenyl](2′-amino-1,1′-biphenyl-2-yl)palladium(II) (76.1 mg, 89.0 umol, 0.100 eq) under nitrogen atmosphere. The reaction mixture was stirred at 90° C. for 16 h under nitrogen atmosphere. The resulting mixture was filtered over celite and the filtrate was added water (50 ml) and extracted with ethyl acetate (3×50 mL). Combined extracts were washed with brine (50 mL), dried over sodium sulfate and filtered. The filtrate was concentrated under reduced pressure to give a residue. The residue was purified by reverse phase chromatography (C18, 80 g; condition: water/acetonitrile=100:0 to 0:100, 0.1% formic acid) and lyophilized to afford tert-butyl (1-(3,5-dichloro-4-(2,6-dioxopiperidin-3-yl)phenyl)azetidin-3-yl)carbamate (87.0 mg, 201 umol, 22% yield, 99% purity) as a white solid.

¹H NMR (400 MHz, DMSO-d₆) δ=10.87 (s, 1H), 7.56 (br d, J=7.6 Hz, 1H), 6.50 (dd, J=2.2, 14.2 Hz, 2H), 4.40 (dd, J=5.6, 12.6 Hz, 2H), 4.10 (t, J=7.6 Hz, 2H), 3.62 (t, J=6.8 Hz, 2H), 2.89-2.76 (m, 1H), 2.52 (br s, 1H), 2.30 (dq, J=4.4, 13.3 Hz, 1H), 1.89-1.79 (m, 1H), 1.39 (s, 9H).

### Step 6. Procedure for Preparation of Compound 7—3-(4-(3-aminoazetidin-1-yl)-2,6-dichlorophenyl)piperidine-2,6-dione

To mixture of tert-butyl (1-(3,5-dichloro-4-(2,6-dioxopiperidin-3-yl)phenyl)azetidin-3-yl)carbamate (87.0 mg, 203 umol, 1.00 eq) in trifluoroacetic acid (1.00 mL) and dichloromethane (5.00 mL). The mixture was stirred at 25° C. for 2 h. The mixture was concentrated under reduced pressure to afford 3-(4-(3-aminoazetidin-1-yl)-2,6-dichlorophenyl)piperidine-2,6-dione (55.0 mg, crude) as yellow oil. MS (ESI) m/z 328.1 [M+H]⁺

### Step 7. Procedure for Preparation of Compound 7A—spiro[3.3]heptan-2-ylmethyl carbonochloridate

To mixture of spiro[3.3]heptan-2-ylmethanol (60.0 mg, 475 umol, 1.00 eq) and bis(trichloromethyl) carbonate (212 mg, 713 umol, 1.50 eq) in dichloromethane (2.00 mL) was added N,N-diisopropylethylamine (123 mg, 951 umol, 166 uL, 2.00 eq) at 0° C. The mixture was stirred at 25° C. for 1 h. The mixture was concentrated under reduced pressure to afford spiro[3.3]heptan-2-ylmethyl carbonochloridate (60.0 mg, crude) as yellow oil.

### Step 8. Procedure for Preparation of spiro[3.3]heptan-2-ylmethyl (1-(3,5-dichloro-4-(2,6-dioxopiperidin-3-yl)phenyl)azetidin-3-yl)carbamate

To mixture of 3-(4-(3-aminoazetidin-1-yl)-2,6-dichlorophenyl)piperidine-2,6-dione (55.0 mg, 168 umol, 1.00 eq) in dichloromethane (2.00 mL) was added N,N-diisopropylethylamine (43.3 mg, 335 umol, 58.4 uL, 2.00 eq) at 0° C. for 15 min. Then was added spiro[3.3]heptan-2-ylmethyl carbonochloridate (34.8 mg, 184 umol, 1.10 eq). The mixture was stirred at 25° C. for 2 h. The mixture was concentrated under reduced pressure to give a residue. The residue was purified by reverse phase chromatography (C18, 80 g; condition: water/acetonitrile=100:0 to 0:100, 0.1% formic acid) and lyophilized to afford spiro[3.3]heptan-2-ylmethyl (1-(3,5-dichloro-4-(2,6-dioxopiperidin-3-yl)phenyl)azetidin-3-yl)carbamate (17.57 mg, 36.2 umol, 21% yield, 99% purity) as a white solid.

¹H NMR (400 MHz, DMSO-d₆) δ=10.87 (s, 1H), 7.82 (br d, J=7.4 Hz, 1H), 6.54 (d, J=2.0 Hz, 1H), 6.50 (d, J=2.0 Hz, 1H), 4.45-4.36 (m, 2H), 4.12 (t, J=7.6 Hz, 2H), 3.90 (d, J=7.0 Hz, 2H), 3.66 (br t, J=6.6 Hz, 2H), 2.88-2.77 (m, 1H), 2.53-2.52 (m, 1H), 2.40-2.31 (m, 2H), 2.04-1.94 (m, 4H), 1.91-1.84 (m, 3H), 1.77-1.68 (m, 4H). MS (ESI) m/z 480.3 [M+H]⁺

### Example 29. Synthesis of Compound 29

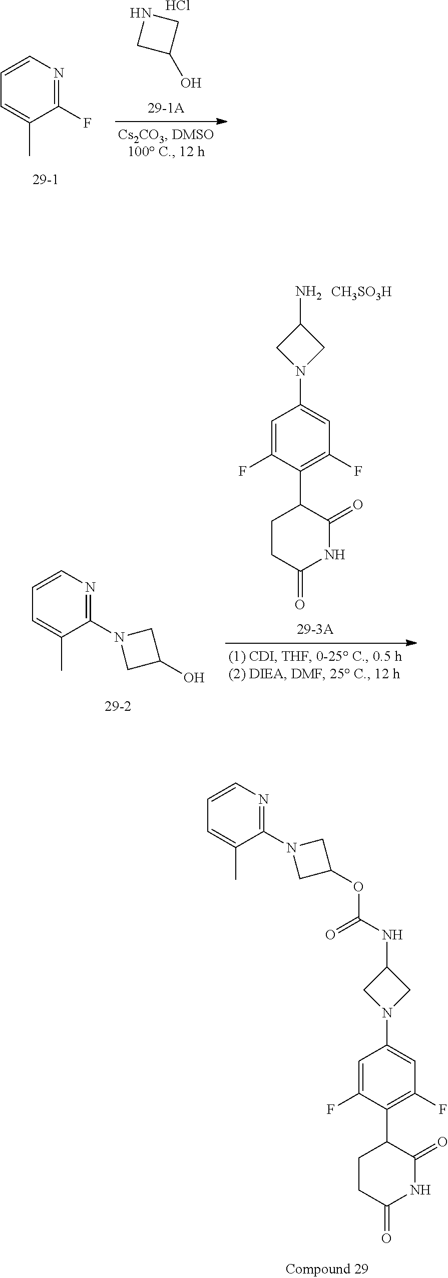

### Step 1. Procedure for Preparation of Compound 2—1-(3-methylpyridin-2-yl)azetidin-3-ol

To the solution of azetidin-3-ol (985 mg, 9.00 mmol, 2.00 eq, hydrochloride), cesium carbonate (4.40 g, 13.5 mmol, 3.00 eq) in dimethylsulfoxide (10.0 mL) was added 2-fluoro-3-methylpyridine (500 mg, 4.50 mmol, 454 uL, 1.00 eq). Then the reaction was stirred at 100° C. for 12 h. The mixture was diluted with water (20 mL), extracted with ethyl acetate (2×10 mL). The combined organic layers were washed with brine (10 mL), and dried over anhydrous sodium sulfate, filtered and concentrate under reduced pressure to give a residue. The residue was purified by column chromatography (SiO₂, petroleum ether/ethyl acetate=5/1 to 0/1) to afford 1-(3-methylpyridin-2-yl)azetidin-3-ol (330 mg, 2.01 mmol, 44% yield) as a yellow solid.

¹H NMR (400 MHz, CDCl₃) δ=8.02 (dd, J=1.1, 4.9 Hz, 1H), 7.23 (dd, J=0.7, 7.2 Hz, 1H), 6.64 (dd, J=5.1, 7.2 Hz, 1H), 4.72-4.60 (m, 1H), 4.36-4.32 (m, 2H), 3.94 (dd, J=4.8, 9.4 Hz, 2H), 3.18-2.81 (m, 1H), 2.17 (s, 3H).

### Step 2. Procedure for Preparation of 1-(3-methylpyridin-2-yl)azetidin-3-yl (1-(4-(2,6-dioxopiperidin-3-yl)-3,5-difluorophenyl)azetidin-3-yl)carbamate

To the solution of 1-(3-methylpyridin-2-yl)azetidin-3-ol (50 mg, 304 umol, 1.00 eq) in tetrahydrofuran (0.50 mL) was added di(1H-imidazol-1-yl)methanone (59.3 mg, 365 umol, 1.20 eq) at 0° C. Then the reaction was stirred at 25° C. for 0.5 h. Then to the mixture were added dimethylformamide (0.50 mL), 3-(4-(3-aminoazetidin-1-yl)-2,6-difluorophenyl)piperidine-2,6-dione (119 mg, 304 umol, 1.00 eq, mesylate), N,N-diisopropylethylamine (59.0 mg, 456 umol, 79.6 uL, 1.50 eq). Then the reaction was stirred at 25° C. for 12 h. The reaction mixture was concentrated under reduced pressure to give a residue. The residue was purified by column chromatography (SiO₂, petroleum ether/ethyl acetate=1/2 to 0/1) to afford 1-(3-methylpyridin-2-yl)azetidin-3-yl (1-(4-(2,6-dioxopiperidin-3-yl)-3,5-difluorophenyl)azetidin-3-yl)carbamate (68.02 mg, 140.11 umol, 44% yield, 98% purity) as a white solid.

¹H NMR (400 MHz, DMSO-d₆) δ=10.87-10.84 (m, 1H), 8.09 (br d, J=7.4 Hz, 1H), 7.96 (br d, J=4.0 Hz, 1H), 7.32 (d, J=7.0 Hz, 1H), 6.68 (dd, J=4.9, 7.1 Hz, 1H), 6.15 (d, J=11.0 Hz, 2H), 5.16-5.09 (m, 1H), 4.47-4.40 (m, 1H), 4.32 (dd, J=6.6, 9.3 Hz, 2H), 4.10 (t, J=7.7 Hz, 2H), 4.03 (br dd, J=4.8, 12.8 Hz, 1H), 3.91 (br dd, J=4.2, 9.3 Hz, 2H), 3.65 (br t, J=6.7 Hz, 2H), 2.82-2.73 (m, 1H), 2.48 (br d, J=3.0 Hz, 1H), 2.12 (s, 3H), 2.10-2.02 (m, 1H), 1.97-1.90 (m, 1H). MS (ESI) m/z 486.3 [M+H]⁺

### Example 30. Synthesis of Compound 30

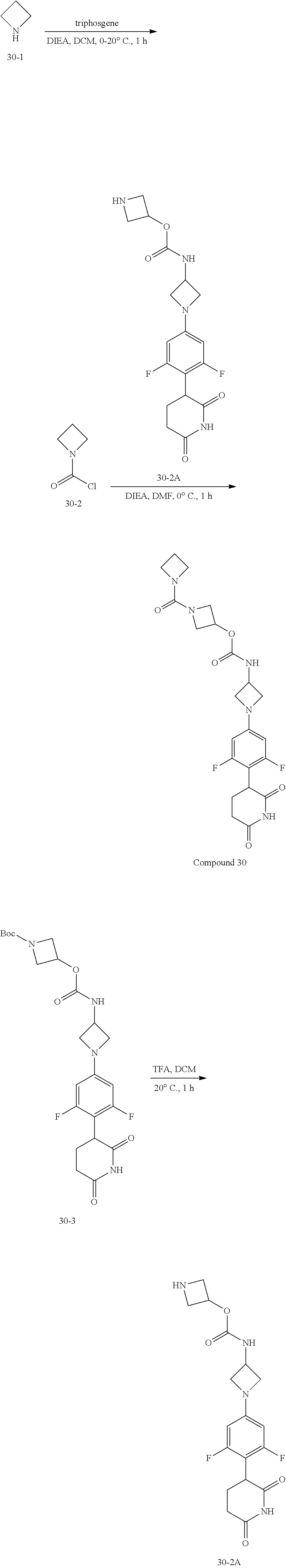

### Step 1. Procedure for Preparation of Compound 2—azetidine-1-carbonyl chloride

To a solution of azetidine (43.0 mg, 460 umol, 50.8 uL, 1.00 eq, hydrochloride) in dichloromethane (3.00 mL) were added bis(trichloromethyl) carbonate (205 mg, 689 umol, 1.50 eq) and N,N-diisopropylethylamine (89.1 mg, 689 umol, 120 uL, 1.50 eq) at 0° C. The reaction was stirred at 20° C. for 1 h. The reaction mixture was concentrated under reduced pressure to afford azetidine-1-carbonyl chloride (54.0 mg, crude) as yellow oil.

### Step 2. Procedure for Preparation of Compound 2A—azetidin-3-yl (1-(4-(2,6-dioxopiperidin-3-yl)-3,5-difluorophenyl)azetidin-3-yl)carbamate

To a solution of tert-butyl 3-(((1-(4-(2,6-dioxopiperidin-3-yl)-3,5-difluorophenyl)azetidin-3-yl)carbamoyl)oxy)azetidine-1-carboxylate (200 mg, 404 umol, 1.00 eq) in dichloromethane (5.00 mL) was added trifluoroacetic acid (1.54 g, 13.5 mmol, 1.00 mL, 33.4 eq). The reaction mixture was stirred at 20° C. for 1 h. The reaction mixture was concentrated under reduced pressure to afford azetidin-3-yl (1-(4-(2,6-dioxopiperidin-3-yl)-3,5-difluorophenyl)azetidin-3-yl)carbamate (205 mg, 403 umol, 100% yield, trifluoroacetate) as yellow oil. MS (ESI) m/z. 395.1 [M+H]⁺

### Step 3. Procedure for Preparation of 1-(azetidine-1-carbonyl)azetidin-3-yl (1-(4-(2,6-dioxopiperidin-3-yl)-3,5-difluorophenyl)azetidin-3-yl)carbamate

To a solution of azetidin-3-yl (1-(4-(2,6-dioxopiperidin-3-yl)-3,5-difluorophenyl)azetidin-3-yl)carbamate (205 mg, 403 umol, 1.00 eq, trifluoroacetate) in dimethylformamide (2.00 mL) were added N,N-diisopropylethylamine (104 mg, 806 umol, 140 μL, 2.00 eq) and azetidine-1-carbonyl chloride (53.0 mg, 444 umol, 1.10 eq) at 0° C. The reaction mixture was stirred at 0° C. for 1 h. The reaction mixture was filtered. The filtrate was purified by Prep-HPLC (column: Phenomenex luna C18 150*25 mm*10 um; mobile phase: [water (formic acid)-acetonitrile]; B %: 20%-50%, 10 min) and lyophilized to give a crude product. The crude product was purified by Prep-TLC (petroleum ether:ethyl acetate=2:1, R_{f}=0.08) and concentrated under reduced pressure to give a white solid. The white solid was purified by reverse phase chromatography (C18, 40 g; condition: water/acetonitrile=100:0 to 30:60, 0.1% formic acid) and lyophilized to afford 1-(azetidine-1-carbonyl)azetidin-3-yl (1-(4-(2,6-dioxopiperidin-3-yl)-3,5-difluorophenyl)azetidin-3-yl)carbamate (8.57 mg, 17.77 umol, 4% yield, 99% purity) as a white solid.

¹H NMR (400 MHz, DMSO-d₆)=10.86 (s, 1H), 8.11 (br d, J=7.1 Hz, 1H), 6.15 (d, J=11.0 Hz, 2H), 5.10-4.98 (m, 1H), 4.46-4.38 (m, 1H), 4.15-4.06 (m, 4H), 4.03 (br dd, J=5.3, 12.4 Hz, 1H), 3.81 (t, J=7.6 Hz, 4H), 3.72-3.61 (m, 4H), 2.83-2.72 (m, 1H), 2.63-2.52 (m, 1H), 2.19-2.11 (m, 2H), 2.10-2.01 (m, 1H), 1.99-1.90 (m, 1H). MS (ESI) m/z. 477.7 [M+H]⁺

### Example 31. Synthesis of Compound 31

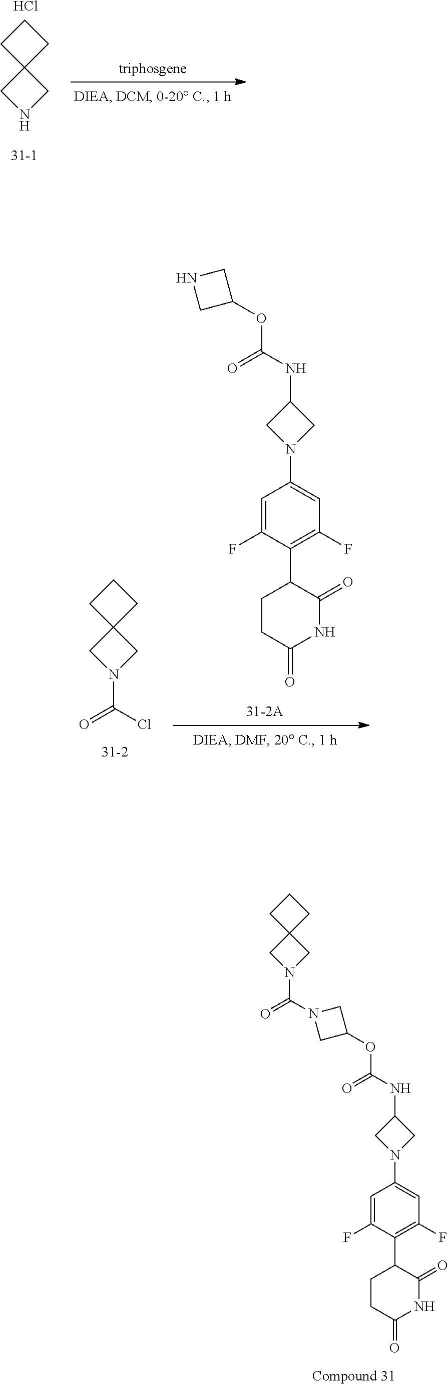 [SMILES: Cl ; C1CC2(C1)CNC2 ; "triphosgene" ; O=C(Cl)N1CC2(CCC2)C1 ; O=C1CCC(c2c(F)cc(N3CC(NC(=O)OC4CNC4)C3)cc2F)C(=O)N1 ; O=C1CCC(c2c(F)cc(N3CC(NC(=O)OC4CN(C(=O)N5CC6(CCC6)C5)C4)C3)cc2F)C(=O)N1]

### Step 1. Procedure for Compound 2—2-azaspiro[3.3]heptane-2-carbonyl chloride

To a solution of 2-azaspiro[3.3]heptane (75.0 mg, 561 umol, 1.00 eq, hydrochloride) in dichloromethane (3.00 mL) were added bis(trichloromethyl) carbonate (250 mg, 842 umol, 1.50 eq) and N,N-diisopropylethylamine (109 mg, 842 umol, 147 uL, 1.50 eq) at 0° C. The reaction mixture was stirred at 20° C. for 1 h. The reaction mixture was concentrated under reduced pressure to afford 2-azaspiro[3.3]heptane-2-carbonyl chloride (89.6 mg, crude) as yellow oil.

### Step 2. Procedure for 1-(2-azaspiro[3.3]heptane-2-carbonyl)azetidin-3-yl (1-(4-(2,6-dioxopiperidin-3-yl)-3,5-difluorophenyl)azetidin-3-yl)carbamate

To a solution of azetidin-3-yl-(1-(4-(2,6-dioxopiperidin-3-yl)-3,5-difluorophenyl)azetidin-3-yl)carbamate (199 mg, 505 umol, 1.00 eq, trifluoroacetate) in dimethyformamide (1.00 mL) were added N,N-diisopropylethylamine (130 mg, 1.01 mmol, 176 uL, 2.00 eq) and 2-azaspiro[3.3]heptane-2-carbonyl chloride (88.6 mg, 555 umol, 1.10 eq). The reaction mixture was stirred at 20° C. for 1 h. The reaction mixture was concentrated under reduced pressure to afford residue. The residue was purified by Prep-HPLC (column: Phenomenex luna C18 150*25 mm*10 um; mobile phase: [water (formic acid)-acetonitrile]; B %: 19%-49%, 15 min) and lyophilized to afford 1-(2-azaspiro[3.3]heptane-2-carbonyl)azetidin-3-yl-(1-(4-(2,6-dioxopiperidin-3-yl)-3,5-difluorophenyl)azetidin-3-yl)carbamate (17.95 mg, 33.99 umol, 7% yield, 98% purity) as a white solid.

¹H NMR (400 MHz, DMSO-d₆) δ=10.86 (s, 1H), 8.16-8.02 (m, 1H), 6.16 (br d, J=11.1 Hz, 2H), 5.02 (br d, J=3.4 Hz, 1H), 4.53-4.33 (m, 1H), 4.18-4.00 (m, 5H), 3.74-3.60 (m, 4H), 2.87-2.73 (m, 1H), 2.60-2.54 (m, 1H), 2.15-2.02 (m, 5H), 1.99-1.90 (m, 1H), 1.82-1.68 (m, 2H). MS (ESI) m/z. 518.1 [M+H]⁺

### Example 32. Synthesis of Compound 32

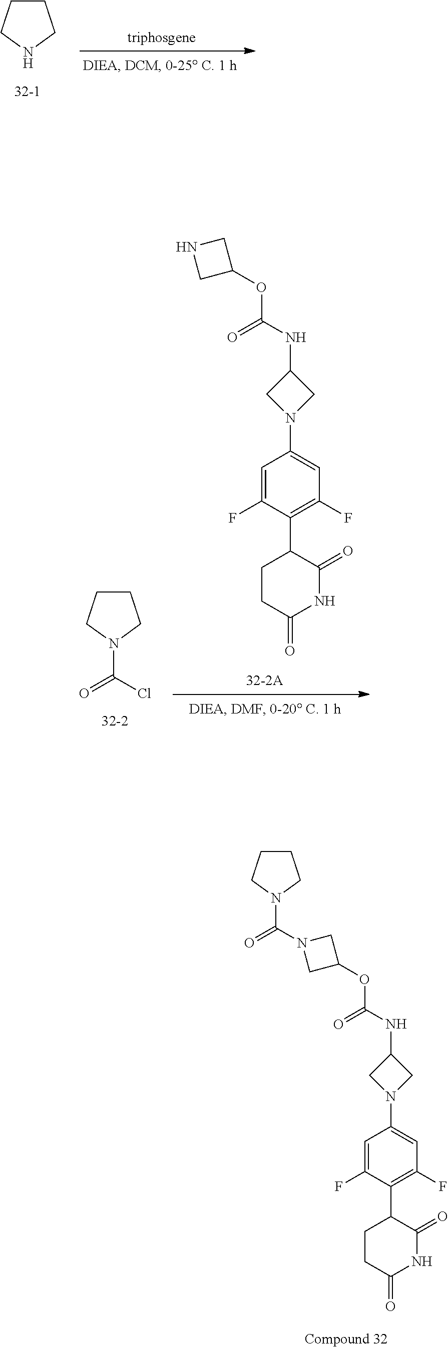 [SMILES: C1CCNC1 ; "triphosgene" ; O=C(Cl)N1CCCC1 ; O=C1CCC(c2c(F)cc(N3CC(NC(=O)OC4CNC4)C3)cc2F)C(=O)N1 ; O=C1CCC(c2c(F)cc(N3CC(NC(=O)OC4CN(C(=O)N5CCCC5)C4)C3)cc2F)C(=O)N1]

### Step 1. Procedure for Preparation of Compound 2—pyrrolidine-1-carbonyl chloride

To a solution of pyrrolidine (30.0 mg, 421 umol, 35.2 uL, 1.00 eq) in dichloromethane (1.00 mL) were added bis(trichloromethyl) carbonate (187 mg, 632 umol, 1.50 eq) and N,N-diisopropylethylamine (81.8 mg, 633 umol, 110 uL, 1.50 eq) at 0° C. The mixture was stirred at 25° C. for 1 h. The reaction mixture was concentrated under reduce pressure to afford pyrrolidine-1-carbonyl chloride (60.0 mg, crude) as a white solid.

### Step 2. Procedure for Preparation of 1-(pyrrolidine-1-carbonyl)azetidin-3-yl (1-(3-fluoro-4-(6-oxopiperidin-3-yl)phenyl)azetidin-3-yl)carbamate

To a solution of azetidin-3-yl (1-(4-(2,6-dioxopiperidin-3-yl)-3,5-difluorophenyl)azetidin-3-yl)carbamate (160 mg, 252 umol, 80% purity, 1.00 eq, trifluoroacetate) in dimethylformamide (2.00 mL) were added N,N-diisopropylethylamine (65.1 mg, 504 umol, 87.7 uL, 2.00 eq) and pyrrolidine-1-carbonyl chloride (36.9 mg, 277 umol, 30.6 uL, 1.10 eq) at 0° C. The reaction mixture was stirred at 20° C. for 1 h. The reaction mixture was filtered and the filtrate was concentrated under reduce pressure to give a residue. The residue was purified by Prep-HPLC (column: Phenomenex luna C18 150*25 mm*10 um; mobile phase: [water (formic acid)-acetonitrile]; B %: 29%-59%, 9 min) and lyophilized to afford 1-(pyrrolidine-1-carbonyl)azetidin-3-yl (1-(3-fluoro-4-(6-oxopiperidin-3-yl)phenyl)azetidin-3-yl)carbamate (8.78 mg, 17.15 umol, 6% yield, 96% purity) as a white solid

¹H NMR (400 MHz, DMSO-d₆) δ=10.85 (s, 1H), 8.10 (br d, J=7.4 Hz, 1H), 6.15 (d, J=11.0 Hz, 2H), 5.01 (tt, J=4.1, 6.6 Hz, 1H), 4.46-4.37 (m, 1H), 4.17-4.07 (m, 4H), 4.03 (br dd, J=5.1, 12.6 Hz, 1H), 3.74 (br dd, J=3.9, 9.5 Hz, 2H), 3.64 (br t, J=6.7 Hz, 2H), 3.19 (br t, J=6.6 Hz, 4H), 2.83-2.72 (m, 1H), 2.49 (br s, 1H), 2.12-2.01 (m, 1H), 1.98-1.90 (m, 1H), 1.78-1.71 (m, 4H). MS (ESI) m/z 492.1 [M+H]⁺

### Example 33. Synthesis of Compound 33

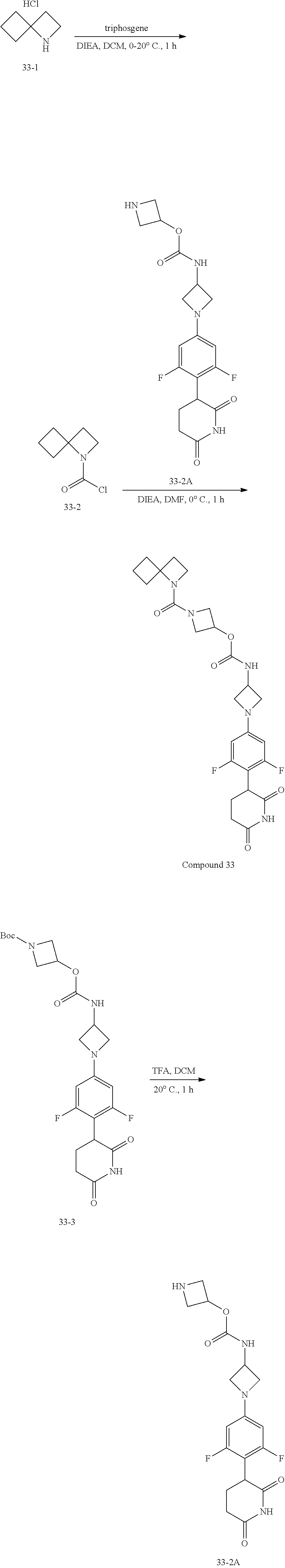

### Step 1. Procedure for Preparation of Compound 2—I-azaspiro[3.3]heptane-1-carbonyl chloride

To a solution of 1-azaspiro[3.3]heptane (45.0 mg, 337 umol, 50.8 uL, 1.00 eq, hydrochloride) in dichloromethane (3.00 mL) were added bis(trichloromethyl) carbonate (150 mg, 505 umol, 1.50 eq) and N,N-diisopropylethylamine (65.3 mg, 505 umol, 88.0 uL, 1.50 eq) at 0° C. The reaction was stirred at 20° C. for 1 h. The reaction mixture was concentrated under reduced pressure to afford 1-azaspiro[3.3]heptane-1-carbonyl chloride (53.0 mg, crude) as yellow oil.

### Step 2. Procedure for Preparation of Compound 2A—azetidin-3-yl (1-(4-(2,6-dioxopiperidin-3-yl)-3,5-difluorophenyl)azetidin-3-yl)carbamate

To a solution of tert-butyl 3-(((1-(4-(2,6-dioxopiperidin-3-yl)-3,5-difluorophenyl)azetidin-3-yl)carbamoyl)oxy)azetidine-1-carboxylate (150 mg, 303 umol, 1.00 eq) in dichloromethane (4.00 mL) was added trifluoroacetic acid (1.23 g, 10.8 mmol, 0.800 mL, 35.6 eq). The reaction mixture was stirred at 20° C. for 1 h. The reaction mixture was concentrated under reduced pressure to afford azetidin-3-yl (1-(4-(2,6-dioxopiperidin-3-yl)-3,5-difluorophenyl)azetidin-3-yl)carbamate (154 mg, 303 umol, 100% yield, trifluoroacetate) as yellow oil. MS (ESI) m/z. 395.1 [M+H]⁺

### Step 3. Procedure for Preparation of 1-(1-azaspiro[3.3]heptane-1-carbonyl)azetidin-3-yl (1-(4-(2,6-dioxopiperidin-3-yl)-3,5-difluorophenyl)azetidin-3-yl)carbamate

To a solution of azetidin-3-yl (1-(4-(2,6-dioxopiperidin-3-yl)-3,5-difluorophenyl)azetidin-3-yl)carbamate (153 mg, 301 umol, 1.00 eq, trifluoroacetate) in dimethylformamide (2.00 mL) were added N,N-diisopropylethylamine (77.8 mg, 602 umol, 105 uL, 2.00 eq) and 1-azaspiro[3.3]heptane-1-carbonyl chloride ((52.8 mg, 331 umol, 1.10 eq) at 0° C. The reaction mixture was stirred at 0° C. for 1 h. The reaction mixture was filtered. The filtrate was purified by reverse phase chromatography (C18, 80 g; condition: water/acetonitrile=100:0 to 0:100, formic acid) and lyophilized to give a crude product. Then the crude product was purified by Prep-HPLC (column: Waters xbridge 150*25 mm*10 um; mobile phase: [water (ammonium bicarbonate)-acetonitrile]; B %: 22%-52%, 8 min), added formic acid (20 L) and lyophilized to afford 1-(1-azaspiro[3.3]heptane-1-carbonyl)azetidin-3-yl (1-(4-(2,6-dioxopiperidin-3-yl)-3,5-difluorophenyl)azetidin-3-yl)carbamate (8.35 mg, 15.33 umol, 5% yield, 95% purity) as a white solid.

¹H NMR (400 MHz, DMSO-d₆)=10.86 (s, 1H), 8.11 (br d, J=7.1 Hz, 1H), 6.15 (br d, J=11.3 Hz, 2H), 5.05-4.95 (m, 1H), 4.48-4.35 (m, 1H), 4.15-4.07 (m, 4H), 4.03 (br dd, J=4.9, 12.4 Hz, 1H), 3.79-3.68 (m, 4H), 3.64 (br t, J=6.4 Hz, 2H), 2.82-2.64 (m, 4H), 2.24 (br t, J=7.2 Hz, 2H), 2.11-2.02 (m, 1H), 1.99-1.87 (m, 3H), 1.69-1.59 (m, 1H), 1.57-1.49 (m, 1H). MS (ESI) m/z. 518.4 [M+H]⁺

### Example 34. Synthesis of Compound 34

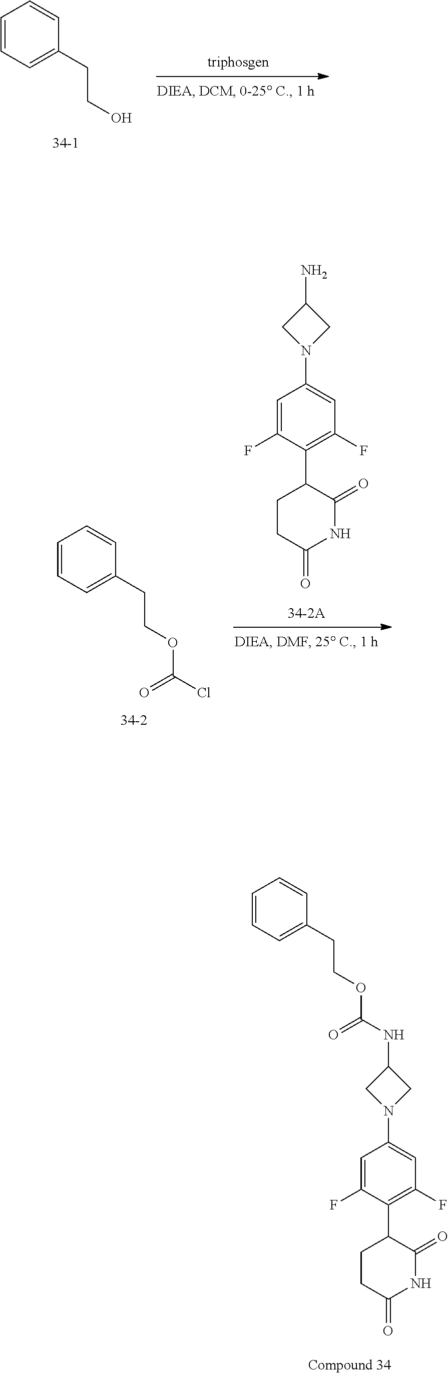 [SMILES: OCCc1ccccc1 ; "triphosgen" ; O=C(Cl)OCCc1ccccc1 ; NC1CN(c2cc(F)c(C3CCC(=O)NC3=O)c(F)c2)C1 ; O=C1CCC(c2c(F)cc(N3CC(NC(=O)OCCc4ccccc4)C3)cc2F)C(=O)N1]

### Step 1. Procedure for Preparation of Compound 2—phenethyl carbonochloridate

To a solution of 2-phenylethan-1-ol (400 mg, 3.27 mmol, 392 uL, 1.00 eq) in dichloromethane (2.00 mL) were added bis(trichloromethyl) carbonate (1.46 g, 4.91 mmol, 1.50 eq) and N,N-diisopropylethylamine (1.27 g, 9.82 mmol, 1.71 mL, 3.00 eq) at 0° C. The mixture was stirred at 25° C. for 1 h. The reaction mixture was filtered and concentrated under reduced pressure to afford phenethyl carbonochloridate (300 mg, crude) as a yellow solid.

### Step 2. Procedure for Preparation of phenethyl (1-(4-(2,6-dioxopiperidin-3-yl)-3,5-difluorophenyl)azetidin-3-yl)carbamate

To a solution of 3-(4-(3-aminoazetidin-1-yl)-2,6-difluorophenyl)piperidine-2,6-dione (100 mg, 256 umol, 1.00 eq, mesylate) in dichloromethane (5.00 mL) were added N,N-diisopropylethylamine (99.1 mg, 767 umol, 134 uL, 3.00 eq) and phenethyl carbonochloridate (70.8 mg, 383 umol, 1.50 eq). The mixture was stirred at 25° C. for 1 h. The reaction mixture was filtered and concentrated under reduced pressure to give a residue. The residue was purified by reverse phase chromatography (C18, 40 g; condition: water/acetonitrile=100:0 to 0:100, 0.1% formic acid) and lyophilized to afford phenethyl (1-(4-(2,6-dioxopiperidin-3-yl)-3,5-difluorophenyl)azetidin-3-yl)carbamate (18.41 mg, 40.69 umol, 16% yield, 98% purity) as a light yellow solid.

¹H NMR (400 MHz, DMSO-d₆) δ=10.85 (s, 1H), 7.84 (br d, J=7.1 Hz, 1H), 7.34-7.21 (m, 5H), 6.14 (br d, J=11.3 Hz, 2H), 4.41 (br dd, J=6.1, 7.2 Hz, 1H), 4.18 (t, J=6.9 Hz, 2H), 4.12-4.05 (m, 2H), 4.02 (br d, J=5.3 Hz, 1H), 3.63 (br t, J=6.1 Hz, 2H), 2.87 (t, J=6.8 Hz, 2H), 2.81-2.71 (m, 1H), 2.48 (br s, 1H), 2.13-2.02 (m, 1H), 1.99-1.91 (m, 1H). MS (ESI) m/z 444.3 [M+H]⁺

### Example 35. Synthesis of Compound 35

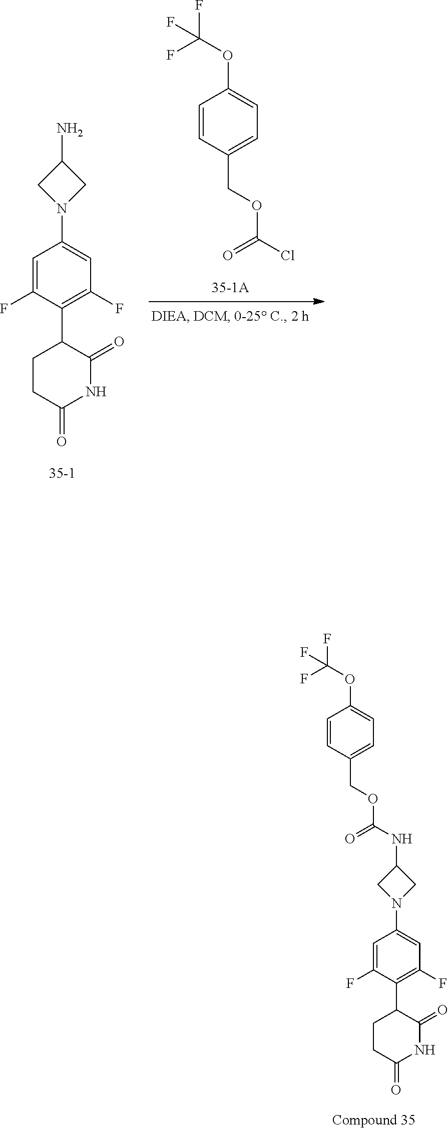 [SMILES: NC1CN(c2cc(F)c(C3CCC(=O)NC3=O)c(F)c2)C1 ; O=C(Cl)OCc1ccc(OC(F)(F)F)cc1 ; O=C1CCC(c2c(F)cc(N3CC(NC(=O)OCc4ccc(OC(F)(F)F)cc4)C3)cc2F)C(=O)N1]

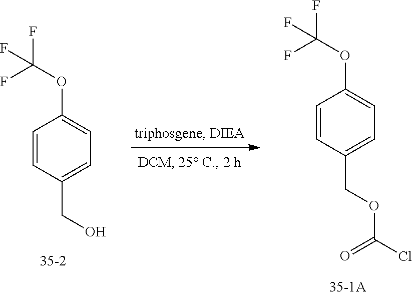 [SMILES: OCc1ccc(OC(F)(F)F)cc1 ; O=C(Cl)OCc1ccc(OC(F)(F)F)cc1]

### Step 1. Procedure for Compound 1A—4-(trifluoromethoxy)benzyl carbonochloridate

To a solution of (4-(trifluoromethoxy)phenyl)methanol (500 mg, 2.60 mmol, 376 uL, 1.00 eq) in dichloromethane (5.00 mL) were added bis(trichloromethyl) carbonate (1.16 g, 3.90 mmol, 1.50 eq) and N,N-diisopropylethylamine (673 mg, 5.20 mmol, 907 uL, 2.00 eq) at 0° C. The mixture was stirred at 25° C. for 2 h. The reaction mixture was filtered and the filtrate was concentrated under reduced pressure to give 4-(trifluoromethoxy)benzyl carbonochloridate (600 mg, crude) as colorless oil.

### Step 2. Procedure for 4-(trifluoromethoxy)benzyl (1-(4-(2,6-dioxopiperidin-3-yl)-3,5-difluorophenyl)azetidin-3-yl)carbamate

To a solution of 4-(trifluoromethoxy)benzyl carbonochloridate (200 mg, 786 umol, 1.00 eq) in dichloromethane (5.00 mL) were added N,N-diisopropylethylamine (203 mg, 1.57 mmol, 274 uL, 2.00 eq) and 3-(4-(3-aminoazetidin-1-yl)-2,6-difluorophenyl)piperidine-2,6-dione (232 mg, 593 umol, 0.754 eq, mesylate). The mixture was stirred at 25° C. for 2 h. The mixture was filtered and the filtrate was concentrated under reduced pressure to give a residue. The residue was purified by Prep-HPLC (column: Phenomenex Luna C18 150*25 mm*10 um; mobile phase: [water (formic acid)-acetonitrile]; B %: 40%-70%, 8 min) and lyophilized to afford 4-(trifluoromethoxy)benzyl (1-(4-(2,6-dioxopiperidin-3-yl)-3,5-difluorophenyl)azetidin-3-yl)carbamate (33.91 mg, 65.39 umol, 8% yield, 99% purity) as an off-white solid.

¹H NMR (400 MHz, DMSO-d₆) δ=10.85 (s, 1H), 8.02 (br d, J=7.6 Hz, 1H), 7.49 (d, J=8.5 Hz, 2H), 7.37 (d, J=8.4 Hz, 2H), 6.15 (d, J=11.1 Hz, 2H), 5.07 (s, 2H), 4.49-4.40 (m, 1H), 4.10 (t, J=7.6 Hz, 2H), 4.06-4.00 (m, 1H), 3.64 (br t, J=6.6 Hz, 2H), 2.83-2.72 (m, 1H), 2.57-2.52 (m, 1H), 2.12-2.02 (m, 1H), 1.97-1.90 (m, 1H). MS (ESI) m/z 514.0 [M+H]⁺

### Example 36. Synthesis of Compound 36

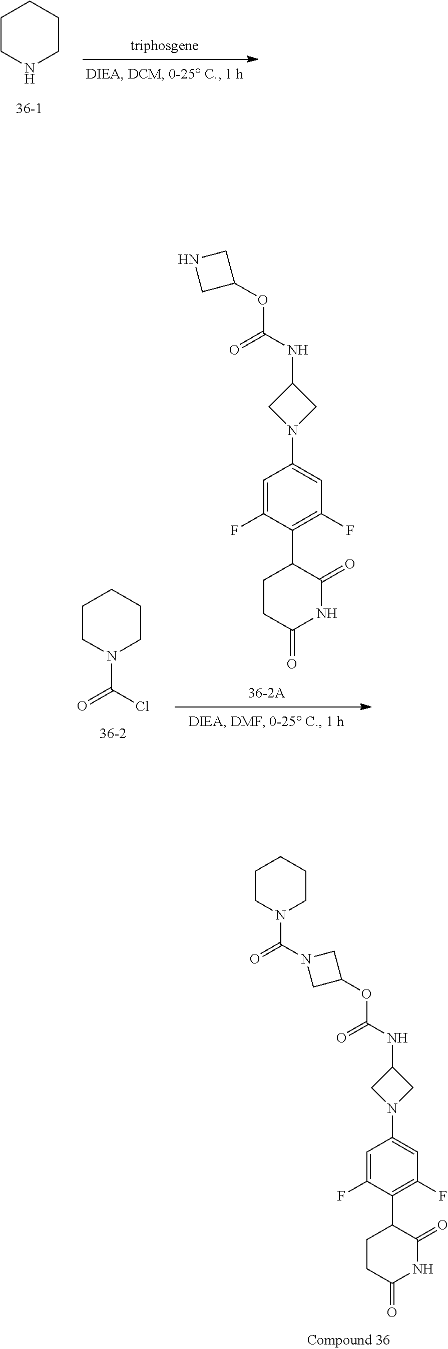 [SMILES: C1CCNCC1 ; "triphosgene" ; O=C(Cl)N1CCCCC1 ; O=C1CCC(c2c(F)cc(N3CC(NC(=O)OC4CNC4)C3)cc2F)C(=O)N1 ; O=C1CCC(c2c(F)cc(N3CC(NC(=O)OC4CN(C(=O)N5CCCCC5)C4)C3)cc2F)C(=O)N1]

### Step 1. Procedure for Preparation of Compound 2—piperidine-1-carbonyl chloride

To a solution of piperidine (45.0 mg, 528 umol, 52.2 uL, 1.00 eq) in dichloromethane (1.00 mL) were added bis(trichloromethyl) carbonate (235 mg, 792 umol, 1.50 eq) and N,N-diisopropylethylamine (102 mg, 793 umol, 138 uL, 1.50 eq) at 0° C. The mixture was stirred at 25° C. for 1 h. The reaction mixture was concentrated under reduce pressure to afford piperidine-1-carbonyl chloride (62.0 mg, crude) as a yellow solid.

### Step 2. Procedure for Preparation of 1-(piperidine-1-carbonyl)azetidin-3-yl (1-(4-(2,6-dioxopiperidin-3-yl)-3,5-difluorophenyl)azetidin-3-yl)carbamate

To a solution of azetidin-3-yl (1-(4-(2,6-dioxopiperidin-3-yl)-3,5-difluorophenyl)azetidin-3-yl)carbamate (194 mg, 381 umol, 1.00 eq, trifluoroacetate) in dimethylformamide (2.00 mL) were added N,N-diisopropylethylamine (98.7 mg, 763 umol, 133 uL, 2.00 eq) and piperidine-1-carbonyl chloride (62.0 mg, 420 umol, 52.5 uL, 1.10 eq) at 0° C. The reaction mixture was stirred at 25° C. for 1 h. The reaction mixture was concentrated under reduce pressure to give a residue. The residue was purified by Prep-HPLC (column: Phenomenex luna C18 150*25 mm*10 um; mobile phase: [water (formic acid)-acetonitrile]; B %: 29%-59%, 9 min) and lyophilized to afford 1-(piperidine-1-carbonyl)azetidin-3-yl (1-(4-(2,6-dioxopiperidin-3-yl)-3,5-difluorophenyl)azetidin-3-yl)carbamate (19.75 mg, 38.68 umol, 10% yield, 99% purity) as a white solid.

¹H NMR (400 MHz, DMSO-d₆) δ=10.91-10.77 (m, 1H), 8.14-8.03 (m, 1H), 6.15 (br d, J=11.0 Hz, 2H), 5.07-4.93 (m, 1H), 4.48-4.35 (m, 1H), 4.17-4.07 (m, 4H), 4.03 (br dd, J=4.9, 12.7 Hz, 1H), 3.75 (br dd, J=3.8, 9.5 Hz, 2H), 3.64 (br t, J=6.8 Hz, 2H), 3.20-3.14 (m, 4H), 2.82-2.73 (m, 1H), 2.48-2.45 (m, 1H), 2.14-2.03 (m, 1H), 1.99-1.90 (m, 1H), 1.52 (br d, J=4.3 Hz, 2H), 1.42 (br d, J=3.8 Hz, 4H). MS (ESI) m/z 506.2 [M+H]⁺

### Example 37. Synthesis of Compound 37

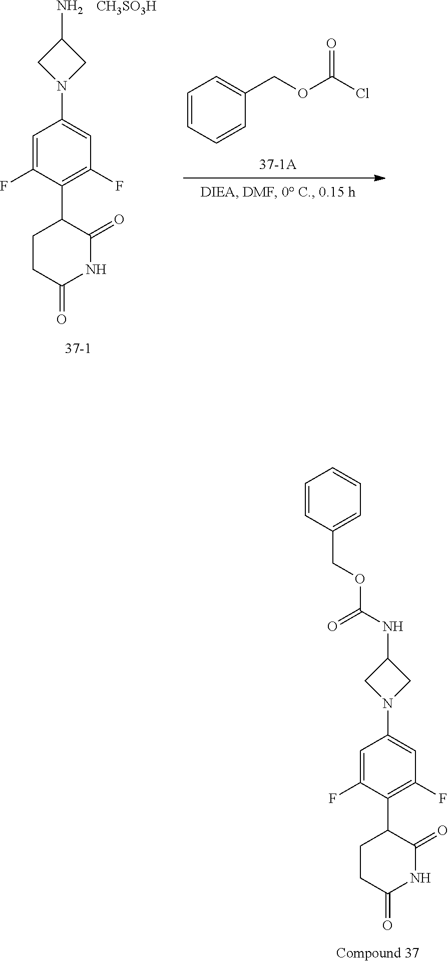

### Step 1. Procedure for Preparation of benzyl (1-(4-(2,6-dioxopiperidin-3-yl)-3,5-difluorophenyl)azetidin-3-yl)carbamate

To a solution of 3-(4-(3-aminoazetidin-1-yl)-2,6-difluorophenyl)piperidine-2,6-dione (300 mg, 767 umol, 1.00 eq, mesylate) in dimethylformamide (2.00 mL) were added N,N-diisopropylethylamine (198 mg, 1.53 mmol, 267 uL, 2.00 eq) and benzyl carbonochloridate (131 mg, 767 umol, 109 uL, 1.00 eq). The reaction mixture was stirred at 0° C. for 0.15 h. The mixture was diluted with water (10 mL) and extracted with ethyl acetate (3×20 mL). The combined organic layers were washed with brine (2×20 mL), dried over anhydrous sodium sulfate, filtered and concentrated under reduced pressure to give a residue. The residue was purified by column chromatography (SiO₂, petroleum ether/ethyl acetate=5/0 to 1/1) and concentrated under reduced pressure to give a solid. The solid was diluted with water (10 mL) and lyophilized to afford benzyl (1-(4-(2,6-dioxopiperidin-3-yl)-3,5-difluorophenyl)azetidin-3-yl)carbamate (237.14 mg, 546.72 umol, 71% yield, 99% purity) as a white solid.

¹H NMR (400 MHz, DMSO-d) δ=10.87 (s, 1H), 7.99 (br d, J=7.4 Hz, 1H), 7.40-7.29 (m, 5H), 6.16 (s, 1H), 6.14 (s, 1H), 5.04 (s, 2H), 4.50-4.40 (m, 1H), 4.10 (t, J=7.6 Hz, 2H), 4.05-4.00 (m, 1H), 3.64 (t, J=6.8 Hz, 2H), 2.82-2.73 (m, 1H), 2.50-2.45 (m, 1H), 2.07 (dq, J=3.7, 12.9 Hz, 1H), 1.98-1.89 (m, 1H). MS (ESI) m/z 430.0 [M+H]⁺

### Example 38. Synthesis of Compound 38

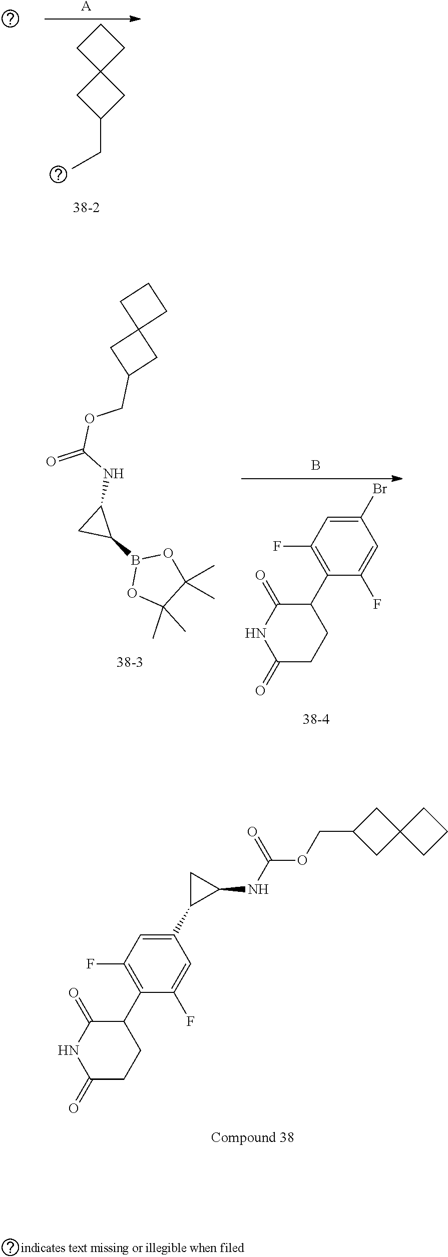

### Step 1

Acid 38-1 (0.5 g, 2.4 mmol) was dissolved in a mixture of compound 38-2/DXN (2/10 mL), then triethylamine (0.4 mL, 3 mmol) and DPPA (0.6 mL, 3 mmol) were added, and the reaction mixture was stirred at 40° C. for 1 h and refluxed overnight. The resulting mixture was cooled to rt, and ethyl acetate (40 mL) was added; the organic layer was washed with 10% K₂CO₃ aq. solution (5×30 mL), and brine (1×20 mL), dried over sodium sulfate, filtered, and concentrated in vacuo to give crude product 38-3 which was used in the next step without further purification (0.3 g, 29%).

¹H NMR (400 MHz, Chloroform-d) δ 4.79 (s, 1H), 3.96 (d, J=7.0 Hz, 2H), 2.74 (s, 1H), 2.38 (s, 1H), 2.07-2.00 (m, 2H), 1.96 (t, J=7.4 Hz, 2H), 1.87 (t, J=7.4 Hz, 2H), 1.80-1.72 (m, 2H), 1.72-1.65 (m, 2H), 1.19 (s, 12H), 0.87 (d, J=5.3 Hz, 1H), 0.83-0.73 (m, 1H), 0.08-0.07 (m, 1H).

### Step 2

To a mixture of compound 38-3 (0.25 g, 0.75 mmol), compound 38-4 (0.23 g, 0.75 mmol), K₂CO₃ (0.2 g, 1.5 mmol) in DXN/H₂O (5:2, 15 mL), purged with argon, cataCXium A Pd G3 (0.03 g) and cataCXium A (0.015 g) were added. The reaction mixture was heated at 90° C. overnight. Then the mixture was cooled to rt and filtered. The solution was concentrated under reduced pressure and the residue was diluted with EtOAc and water. The organic layer was washed with water, brine, dried over Na₂SO₄ and evaporated in vacuo. The residue was purified by HPLC to afford compound 38 (25.5 mg, 8% yield).

¹H NMR (400 MHz, DMSO-d₆) δ 10.94 (s, 1H), 9.33 (d, J=10.1 Hz, 1H), 6.93 (d, J=10.2 Hz, 2H), 6.59-6.37 (m, 1H), 5.25-5.00 (m, 1H), 4.19 (dd, J=12.8, 5.2 Hz, 1H), 3.95 (d, J=6.9 Hz, 2H), 3.28 (d, J=7.1 Hz, 2H), 2.88-2.72 (m, 1H), 2.45-2.28 (m, 2H), 2.16-1.94 (m, 6H), 1.88 (t, J=7.4 Hz, 2H), 1.81-1.63 (m, 4H). HPLC purity: 100%; Ret time: 1.446 min; HRMS (ESI) calculated for C₂₃H₂₆F₂N₂O₄: 432.19; observed: 431.0 [M−H]⁻

### Example 39. Synthesis of Compound 39

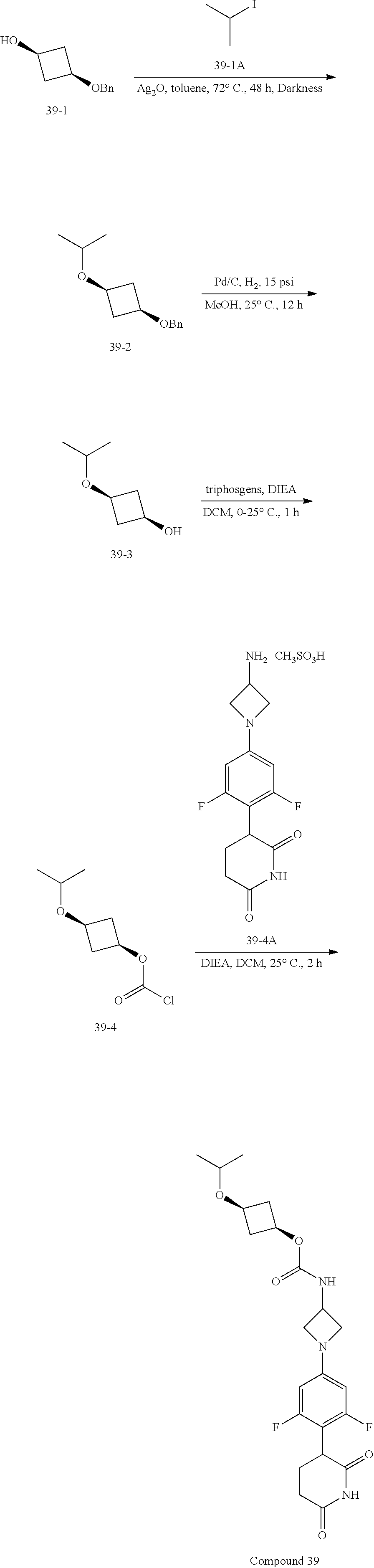

### Step 1. Procedure for Compound 2—(((1s, 3s)-3-isopropoxycyclobutoxy)methyl)benzene

To a solution of (1s, 3s)-3-(benzyloxy)cyclobutanol (200 mg, 1.12 mmol, 1.00 eq) in toluene (5.00 mL) was added 2-iodopropane (382 mg, 2.24 mmol, 224 uL, 2.00 eq). Then argentiooxysilver (520 mg, 2.24 mmol, 2.00 eq) was added to the mixture and the reaction mixture was stirred at 72° C. for 48 h under nitrogen atmosphere and darkness. The mixture was filtered and the filtrate was concentrated under reduced pressure to give a residue. The residue was purified by Prep-TLC (SiO₂, petroleum ether/ethyl acetate=10/1) to afford (((1s, 3s)-3-isopropoxycyclobutoxy)methyl)benzene (100 mg, crude) as colorless oil.

¹H NMR (400 MHz, CDCl₃-d) δ=7.42-7.32 (m, 4H), 7.31-7.27 (m, 1H), 4.44 (s, 2H), 3.72-3.56 (m, 3H), 2.68-2.60 (m, 2H), 2.03-1.94 (m, 2H), 1.15 (d, J=6.1 Hz, 6H).

### Step 2. Procedure for Preparation of Compound 3—(1s, 3s)-3-isopropoxycyclobutanol

To a solution of (((1s, 3s)-3-isopropoxycyclobutoxy)methyl)benzene (50.0 mg, 227 umol, 1.00 eq) in methanol (1.00 mL) was added palladium on activated carbon (100 mg, 10% purity). The mixture was stirred at 25° C. for 12 h under 15 psi of hydrogen atmosphere. The mixture was filtered and the filtrate was concentrated under reduced pressure to afford (1s, 3s)-3-isopropoxycyclobutanol (59.0 mg, crude) as colorless oil.

¹H NMR (400 MHz, CDCl-d) δ=3.95-3.85 (m, 1H), 3.64-3.53 (m, 2H), 2.77-2.66 (m, 2H), 1.99-1.83 (m, 3H), 1.14-1.11 (m, 6H).

### Step 3. Procedure for Compound 4—(1s, 3s)-3-isopropoxycyclobutyl carbonochloridate

To a solution of (1s, 3s)-3-isopropoxycyclobutanol (59.0 mg, 453 umol, 1.00 eq) in dichloromethane (2.00 mL) were added bis(trichloromethyl) carbonate (202 mg, 680 umol, 1.50 eq) and N,N-diisopropylethylamine (117 mg, 906 umol, 158 uL, 2.00 eq) at 0° C. The mixture was stirred at 25° C. for 1 h. The reaction mixture was filtered and concentrated under reduced pressure to afford (1s, 3s)-3-isopropoxycyclobutyl carbonochloridate (87.3 mg, crude) as colorless oil.

### Step 4. Procedure for spiro[3.3]heptan-2-ylmethyl (1-(4-(2,6-dioxopiperidin-3-yl)-3-fluorophenyl)azetidin-3-yl)carbamate

To a solution of 3-(4-(3-aminoazetidin-1-yl)-2,6-difluorophenyl)piperidine-2,6-dione (135 mg, 457 umol, 1.00 eq) in dichloromethane (3.00 mL) were added N,N-diisopropylethylamine (118 mg, 914 umol, 159 uL, 2.00 eq) and (1s, 3s)-3-isopropoxycyclobutyl carbonochloridate (87.0 mg, 452 umol, 0.998 eq). The mixture was stirred at 25° C. for 2 h. The mixture was filtered and the filtrate was concentrated under reduced pressure to give a residue. The residue was purified by Prep-HPLC (column: Phenomenex luna C18 150*25 mm*10 um; mobile phase: [water (formic acid)-acetonitrile]; B %: 34%-64%, min) and lyophilized to afford (1s, 3s)-3-isopropoxycyclobutyl (1-(4-(2,6-dioxopiperidin-3-yl)-3,5-difluorophenyl)azetidin-3-yl)carbamate (12.97 mg, 25.55 umol, 6% yield, 98% purity, formate) an white solid.

¹H NMR (400 MHz, DMSO-d₆) δ=10.85 (br s, 1H), 8.44 (s, 1H), 7.87 (br d, J=7.5 Hz, 1H), 6.14 (d, J=11.1 Hz, 2H), 4.52-4.43 (m, 1H), 4.43-4.34 (m, 1H), 4.11-4.00 (m, 3H), 3.72-3.65 (m, 1H), 3.65-3.58 (m, 2H), 3.57-3.49 (m, 1H), 2.83-2.72 (m, 1H), 2.69-2.61 (m, 2H), 2.47 (br s, 1H), 2.14-2.00 (m, 1H), 1.99-1.89 (m, 1H), 1.87-1.76 (m, 2H), 1.05 (d, J=6.1 Hz, 6H). MS (ESI) m/z 452.1 [M+H]⁺

### Example 40. Synthesis of Compound 40

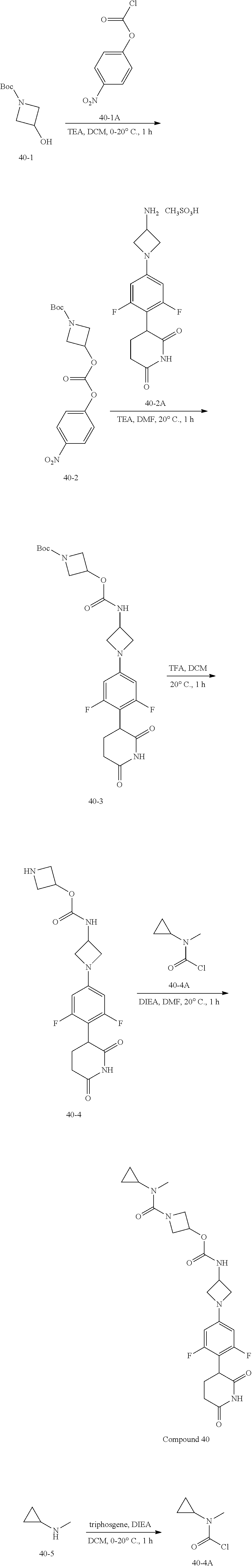

### Step 1. Procedure for Compound 2—tert-butyl 3-(((4-nitrophenoxy)carbonyl)oxy)azetidine-1-carboxylate

To a solution of tert-butyl 3-hydroxyazetidine-1-carboxylate (5.00 g, 28.9 mmol, 1.00 eq) in dichloromethane (50.0 mL) were added 4-nitrophenyl carbonochloridate (20.4 g, 101 mmol, 3.50 eq) and triethylamine (14.6 g, 144 mmol, 20.1 mL, 5.00 eq) at 0° C. The reaction mixture was stirred at 20° C. for 1 h. The reaction mixture was concentrated under reduced pressure. The residue was purified by column chromatography (SiO₂, petroleum ether/ethyl acetate=1/0 to 10/1) to afford tert-butyl 3-(((4-nitrophenoxy)carbonyl)oxy)azetidine-1-carboxylate (9.00 g, 5.32 mmol, 18% yield, 20% purity) as yellow oil.

¹H NMR (400 MHz, CDCl₃-d) δ=8.33-8.26 (m, 2H), 7.42-7.36 (m, 2H), 5.31-5.20 (m, 1H), 4.35 (dd, J=6.7, 10.4 Hz, 2H), 4.10 (dd, J=4.0, 10.5 Hz, 2H), 1.48 (s, 9H).

### Step 2. Procedure for Compound 3—tert-butyl 3-(((1-(4-(2,6-dioxopiperidin-3-yl)-3,5-difluorophenyl)azetidin-3-yl)carbamoyl)oxy)azetidine-1-carboxylate

To a solution of tert-butyl 3-(((4-nitrophenoxy)carbonyl)oxy)azetidine-1-carboxylate (4.49 g, 2.66 mmol, 20% purity, 1.00 eq) in dimethyformamide (10.0 mL) was added 3-(4-(3-aminoazetidin-1-yl)-2,6-difluorophenyl)piperidine-2,6-dione (1.00 g, 2.66 mmol, 1.00 eq, methanesulfonic acid) and triethylamine (538 mg, 5.31 mmol, 740 uL, 2.00 eq). The reaction mixture was stirred at 20° C. for 1 h. The reaction mixture was quenched with water (50 mL) and extracted with ethyl acetate (3×100 mL). The combined organic layers were washed with brine (100 mL), dried over anhydrous sodium sulfate, and concentrated under reduced pressure to give a residue. The residue was purified by column chromatography (SiO₂, petroleum ether/ethyl acetate=10/1 to 0/1) to afford tert-butyl 3-(((1-(4-(2,6-dioxopiperidin-3-yl)-3,5-difluorophenyl)azetidin-3-yl)carbamoyl)oxy)azetidine-1-carboxylate (1.20 g, crude) as a yellow solid.

¹H NMR (400 MHz, CDCl₃-d) δ=8.06 (br s, 1H), 5.97 (d, J=10.4 Hz, 2H), 5.51 (br d, J=7.8 Hz, 1H), 5.20-5.03 (m, 1H), 4.71-4.51 (m, 1H), 4.24-4.17 (m, 4H), 3.93-3.88 (m, 2H), 3.68 (br t, J=5.9 Hz, 2H), 2.85-2.74 (m, 1H), 2.71-2.60 (m, 1H), 2.31 (dq, J=4.6, 12.9 Hz, 1H), 2.17-2.07 (m, 1H), 1.44 (s, 9H).

### Step 3. Procedure for Compound 4—azetidin-3-yl (1-(4-(2,6-dioxopiperidin-3-yl)-3,5-difluorophenyl)azetidin-3-yl)carbamate

To a solution of tert-butyl-3-(((1-(4-(2,6-dioxopiperidin-3-yl)-3,5-difluorophenyl)azetidin-3-yl)carbamoyl) oxy)azetidine-1-carboxylate (250 mg, 506 umol, 1.00 eq) in dichloromethane (5.00 mL) was added trifluoroacetic acid (1.00 mL). The reaction mixture was stirred at 20° C. for 1 h. The reaction mixture was concentrated under reduced pressure to afford azetidin-3-yl (1-(4-(2,6-dioxopiperidin-3-yl)-3,5-difluorophenyl)azetidin-3-yl)carbamate (199 mg, 505 umol, 99% yield) as yellow oil.

¹H NMR (400 MHz, DMSO-d₆) δ=10.82-10.62 (m, 1H), 8.21-8.09 (m, 1H), 6.28-6.18 (m, 1H), 6.08 (d, J=11.0 Hz, 1H), 5.03 (br t, J=6.2 Hz, 1H), 4.35-4.15 (m, 6H), 3.57 (br d, J=7.1 Hz, 1H), 3.52-3.42 (m, 1H), 3.34 (br d, J=5.6 Hz, 1H), 3.23 (br d, J=6.4 Hz, 1H), 3.13-2.90 (m, 1H), 2.74-2.68 (m, 1H), 2.51-2.45 (m, 1H), 2.06-1.94 (m, 1H), 1.91-1.83 (m, 1H).

### Step 4. Procedure for Compound 4A—cyclopropyl(methyl)carbamic chloride

To a solution of N-methylcyclopropanamine (40.0 mg, 562 umol, 1.00 eq) in dichloromethane (3.00 mL) were added bis(trichloromethyl) carbonate (250 mg, 844 umol, 1.50 eq) and N,N-diisopropylethylamine (109 mg, 844 umol, 147 uL, 1.50 eq) at 0° C. The reaction mixture was stirred at 20° C. for 1 h. The reaction mixture was concentrated under reduced pressure to afford cyclopropyl(methyl)carbamic chloride (75.0 mg, 561 umol, 99% yield) as yellow oil.

### Step 5. Procedure for 1-(cyclopropyl(methyl)carbamoyl)azetidin-3-yl (1-(4-(2,6-dioxopiperidin-3-yl)-3,5-difluorophenyl)azetidin-3-yl)carbamate

To a solution of azetidin-3-yl (1-(4-(2,6-dioxopiperidin-3-yl)-3,5-difluorophenyl)azetidin-3-yl)carbamate (199 mg, 505 umol, 1.00 eq) in dimethyformamide (1.00 mL) were added N,N-diisopropylethylamine (130 mg, 1.01 mmol, 176 uL, 2.00 eq) and cyclopropyl(methyl)carbamic chloride (74.1 mg, 555 umol, 1.10 eq). The reaction mixture was stirred at 20° C. for 1 h. The reaction mixture was concentrated under reduced pressure to afford residue. The residue was purified by Prep-HPLC (column: Waters xbridge 150*25 mm 10 um; mobile phase: [water (ammonium bicarbonate)-acetonitrile]; B %: 17%-47%, 9 min). The mixture was added formic acid (0.1 mL) and lyophilized to afford 1-(cyclopropyl(methyl)carbamoyl)azetidin-3-yl (29.91 mg, 59.64 umol, 12% yield, 98% purity) as a white solid.

¹H NMR (400 MHz, DMSO-d₆) δ=10.86 (br s, 1H), 8.11 (br d, J=7.6 Hz, 1H), 6.16 (d, J=11.0 Hz, 2H), 5.12-4.96 (m, 1H), 4.48-4.36 (m, 1H), 4.23 (dd, J=6.6, 9.6 Hz, 2H), 4.10 (br t, J=7.5 Hz, 2H), 4.04 (br dd, J=5.4, 12.7 Hz, 1H), 3.82 (br dd, J=3.8, 9.8 Hz, 2H), 3.70-3.62 (m, 2H), 2.86-2.75 (m, 2H), 2.73 (s, 3H), 2.57 (br d, J=3.4 Hz, 1H), 2.13-2.03 (m, 1H), 2.00-1.89 (m, 1H), 0.77-0.70 (m, 2H), 0.68-0.58 (m, 2H). MS (ESI) m/z. 492.0 [M+H]⁺

### Example 41. Synthesis of Compound 41

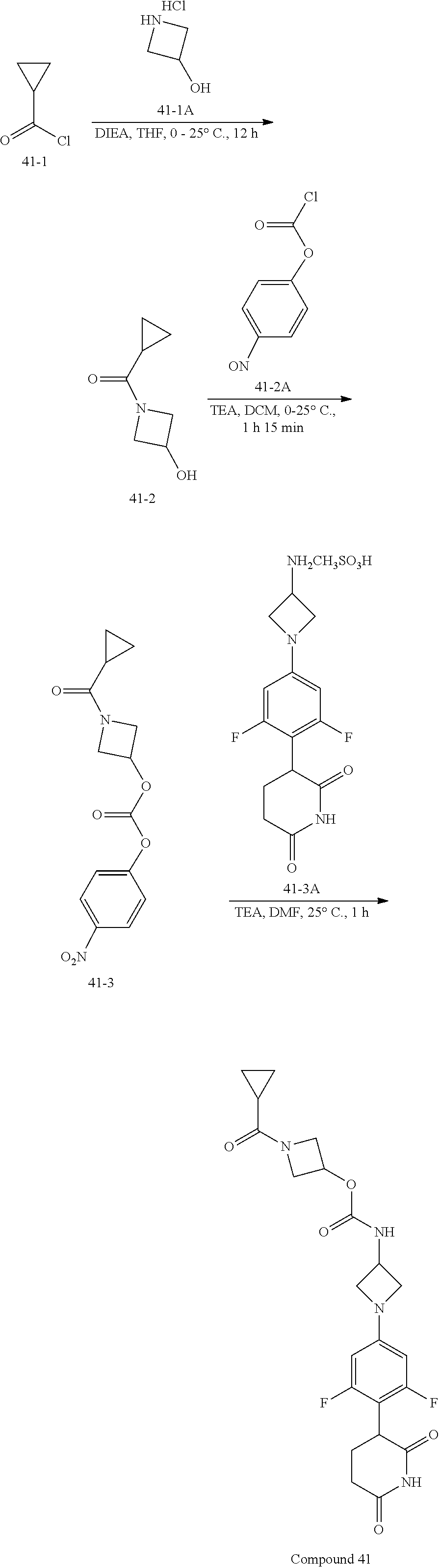

### Step 1. Procedure for Preparation of Compound 2—cyclopropyl(3-hydroxyazetidin-1-yl)methanone

To the solution of azetidin-3-ol (524 mg, 4.78 mmol, 1.00 eq, hydrochloride), N,N-diisopropylethylamine (1.85 g, 14.4 mmol, 2.50 mL, 3.00 eq) in tetrahydrofuran (5.00 mL) was added cyclopropanecarbonyl chloride (500 mg, 4.78 mmol, 435 uL, 1.00 eq) at 0° C. Then the reaction was stirred at 25° C. for 12 h. The reaction mixture was concentrated under reduced pressure to give a residue. The residue was purified by column chromatography (SiO₂, petroleum ether/ethyl acetate=0/1) to afford cyclopropyl(3-hydroxyazetidin-1-yl)methanone (550 mg, 3.90 mmol, 81% yield) as a white solid.

¹H NMR (400 MHz, CDCl₃) δ=4.65 (tt, J=4.4, 6.8 Hz, 1H), 4.49-3.82 (m, 4H), 3.57 (br s, 1H), 1.45-1.37 (m, 1H), 0.97-0.89 (m, 2H), 0.81-0.72 (m, 2H).

### Step 2. Procedure for Preparation of Compound 3—1-(cyclopropanecarbonyl)azetidin-3-yl (4-nitrophenyl) carbonate

To the solution of cyclopropyl(3-hydroxyazetidin-1-yl)methanone (200 mg, 1.42 mmol, 1.00 eq) in dichloromethane (3.00 mL) was added triethylamine (717 mg, 7.08 mmol, 986 uL, 5.00 eq) at 0° C. After 15 min, to the mixture was added 4-nitrophenyl carbonochloridate (857 mg, 4.25 mmol, 3.00 eq). Then the reaction was stirred at 25° C. for 1 h. The reaction mixture was concentrated under reduced pressure to give a residue. The residue was purified by column chromatography (SiO₂, petroleum ether/ethyl acetate=1/1) to afford 1-(cyclopropanecarbonyl)azetidin-3-yl (4-nitrophenyl) carbonate (240 mg, 392 umol, 27% yield, 50% purity) as a yellow solid.

¹H NMR (400 MHz, CDCl₃) δ=8.34-8.28 (m, 2H), 7.43-7.38 (m, 2H), 5.38-5.32 (m, 1H), 4.69-4.49 (m, 2H), 4.25-4.12 (m, 2H), 1.41-1.39 (m, 1H), 1.00-0.97 (m, 2H), 0.78 (br dd, J=3.0, 8.3 Hz, 2H).

### Step 3. Procedure for Preparation of 1-(cyclopropanecarbonyl)azetidin-3-yl (1-(4-(2,6-dioxopiperidin-3-yl)-3,5-difluorophenyl)azetidin-3-yl)carbamate

To the solution of 1-(cyclopropanecarbonyl)azetidin-3-yl (4-nitrophenyl) carbonate (50.0 mg, 163 umol, 1.00 eq), 3-(4-(3-aminoazetidin-1-yl)-2,6-difluorophenyl)piperidine-2,6-dione (63.9 mg, 163 umol, 1.00 eq, methanesulfonic acid) in dimethylformamide (0.500 mL) was added triethylamine (33.1 mg, 327 umol, 45.5 uL, 2.00 eq). Then the reaction was stirred at 25° C. for 1 h. The reaction mixture was concentrated under reduced pressure to give a residue. The residue was purified by Prep-HPLC (column: Phenomenex luna C18 150*25 mm*10 um; mobile phase: [column: Waters xbridge 150*25 mm 10 um; mobile phase: [water (ammonium bicarbonate)-acetonitrile]; B %: 13%-43%, 13 min) and lyophilized to afford 1-(cyclopropanecarbonyl)azetidin-3-yl (1-(4-(2,6-dioxopiperidin-3-yl)-3,5-difluorophenyl)azetidin-3-yl)carbamate (34.37 mg, 73.58 umol, 90% yield, 99% purity) as a white solid.

¹H NMR (400 MHz, DMSO-d₆) δ=10.85 (br s, 1H), 8.15 (br d, J=7.5 Hz, 1H), 6.17 (s, 1H), 6.14 (s, 1H), 5.12-5.07 (m, 1H), 4.56-4.50 (m, 1H), 4.45-4.39 (m, 1H), 4.11 (br d, J=7.6 Hz, 4H), 4.06-4.01 (m, 1H), 3.73-3.69 (m, 1H), 3.65 (br d, J=6.9 Hz, 2H), 2.82-2.73 (m, 1H), 2.48 (br s, 1H), 2.12-2.03 (m, 1H), 1.96-1.90 (m, 1H), 1.55-1.50 (m, 1H), 0.72-0.67 (m, 4H). MS (ESI) m/z 463.1 [M+H]⁺

### Example 42. Synthesis of Compound 42

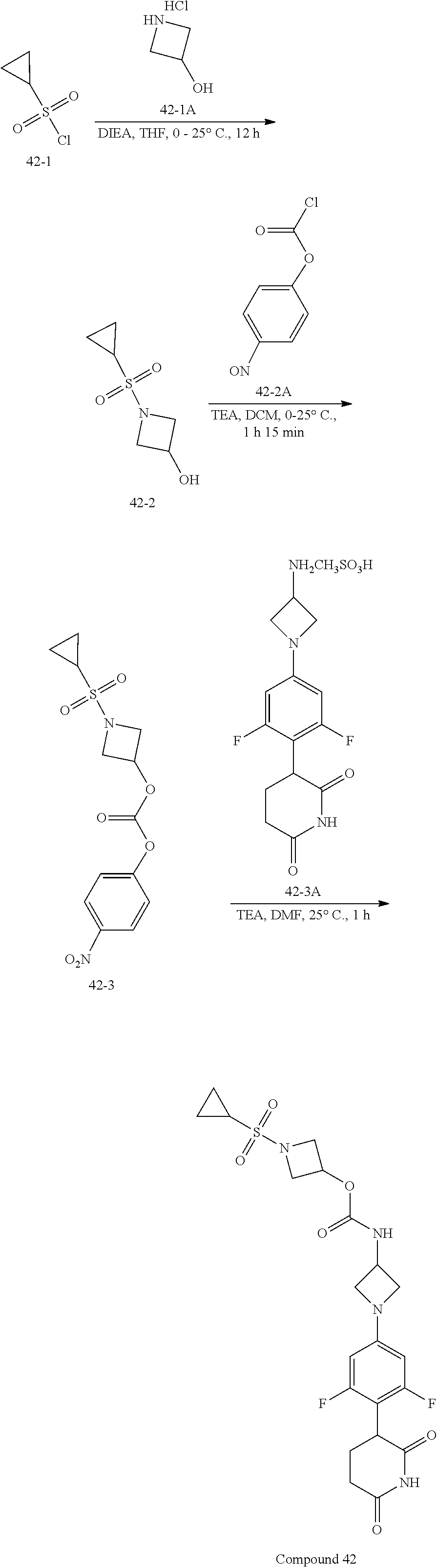

### Step 1. Procedure for Preparation of Compound 2—1-(cyclopropylsulfonyl)azetidin-3-ol

To the solution of azetidin-3-ol (779 mg, 7.11 mmol, 1.00 eq, hydrochloride), N,N-diisopropylethylamine (2.76 g, 21.3 mmol, 3.72 mL, 3.00 eq) in tetrahydrofuran (5.00 mL) was added cyclopropanesulfonyl chloride (1.00 g, 7.11 mmol, 1.00 eq) at 0° C. Then the reaction was stirred at 25° C. for 12 h. The reaction mixture was concentrated under reduced pressure to give a residue. The residue was purified by column chromatography (SiO₂, petroleum ether/ethyl acetate=0/1) to afford 1-(cyclopropylsulfonyl)azetidin-3-ol (1.25 g, 7.05 mmol, 99% yield) as orange oil.

¹H NMR (400 MHz, CDCl₃) δ=4.64-4.57 (m, 1H), 4.11-4.07 (m, 2H), 3.92-3.87 (m, 2H), 2.95 (br s, 1H), 2.38 (tt, J=4.9, 8.0 Hz, 1H), 1.17-1.12 (m, 2H), 1.05-1.00 (m, 2H).

### Step 2. Procedure for Preparation of Compound 3—1-(cyclopropylsulfonyl)azetidin-3-yl (4-nitrophenyl) carbonate

To the solution of 1-(cyclopropylsulfonyl)azetidin-3-ol (400 mg, 2.26 mmol, 1.00 eq) in dichloromethane (5.00 mL) was added triethylamine (1.14 g, 11.3 mmol, 1.57 mL, 5.00 eq) at 0° C. After 15 min, to the mixture was added 4-nitrophenyl carbonochloridate (1.36 g, 6.77 mmol, 3.00 eq). Then the reaction was stirred at 25° C. for 1 h. The reaction mixture was concentrated under reduced pressure to give a residue. The residue was purified by column chromatography (SiO₂, petroleum ether/ethyl acetate=5/1) to afford 1-(cyclopropylsulfonyl)azetidin-3-yl (4-nitrophenyl) carbonate (550 mg, 1.12 mmol, 49% yield, 70% purity) as an off-white solid.

¹H NMR (400 MHz, CDCl₃) δ=8.32-8.27 (m, 2H), 7.42-7.37 (m, 2H), 5.28 (tt, J=5.0, 6.6 Hz, 1H), 4.31-4.25 (m, 2H), 4.04-4.00 (m, 2H), 2.40-2.37 (m, 1H), 1.18 (br dd, J=1.8, 2.9 Hz, 2H), 1.06-1.03 (m, 2H).

### Step 3. Procedure for Preparation of 1-(cyclopropylsulfonyl)azetidin-3-yl (1-(4-(2,6-dioxopiperidin-3-yl)-3,5-difluorophenyl)azetidin-3-yl)carbamate

To the solution of 1-(cyclopropylsulfonyl)azetidin-3-yl (4-nitrophenyl) carbonate (50.0 mg, 146 umol, 1.00 eq), 3-(4-(3-aminoazetidin-1-yl)-2,6-difluorophenyl)piperidine-2,6-dione (57.2 mg, 146 umol, 1.00 eq, methanesulfonic acid) in dimethylformamide (0.500 mL) was added triethylamine (29.6 mg, 292 umol, 40.7 uL, 2.00 eq). Then the reaction was stirred at 25° C. for 1 h. The reaction mixture was concentrated under reduced pressure to give a residue. The residue was purified by Prep-HPLC (column: Waters xbridge 150*25 mm 10 um; mobile phase: [water (formic acid)-acetonitrile]; B %: 20%-80%, 8 min) and lyophilized to afford 1-(cyclopropylsulfonyl)azetidin-3-yl (1-(4-(2,6-dioxopiperidin-3-yl)-3,5-difluorophenyl)azetidin-3-yl)carbamate (13.01 mg, 25.84 umol, 17% yield, 99% purity) as a white solid.

¹H NMR (400 MHz, DMSO-d₆) δ=10.86 (s, 1H), 8.19 (br d, J=7.4 Hz, 1H), 6.17 (s, 1H), 6.15 (s, 1H), 5.12-5.06 (m, 1H), 4.47-4.38 (m, 1H), 4.18-4.09 (m, 4H), 4.04 (br dd, J=5.1, 12.6 Hz, 1H), 3.86 (br dd, J=4.9, 9.1 Hz, 2H), 3.66 (br t, J=6.8 Hz, 2H), 3.30-3.19 (m, 1H), 2.82-2.73 (m, 2H), 2.11-2.03 (m, 1H), 1.98-1.92 (m, 1H), 1.05-1.02 (m, 2H), 0.97-0.93 (m, 2H). MS (ESI) m/z 499.1 [M+H]⁺

### Example 43. Synthesis of Compound 43

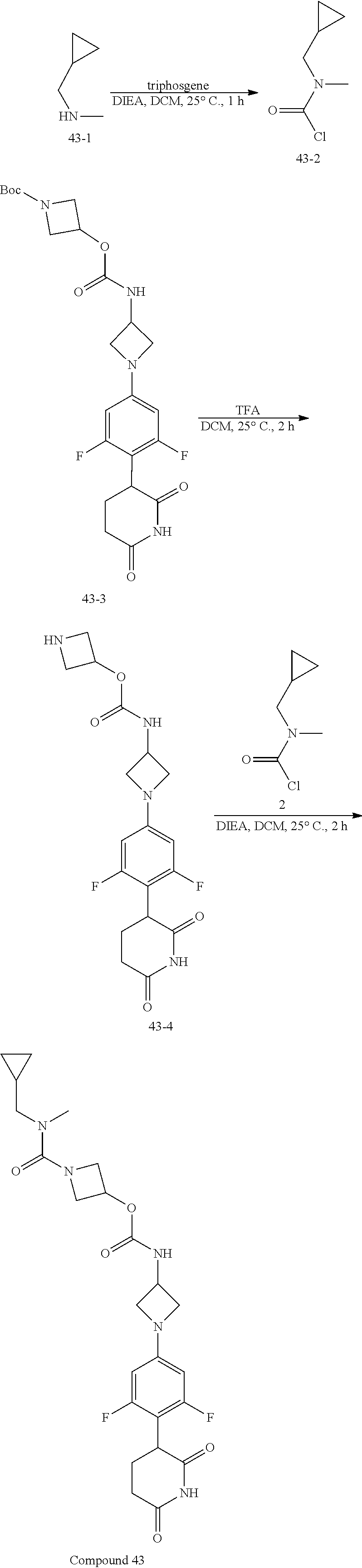

### Step 1. Procedure for Preparation of Compound 2—(cyclopropylmethyl)(methyl)carbamic chloride

To a solution of 1-cyclopropyl-N-methyl-methanamine (35.0 mg, 411 umol, 1.00 eq) in dichloromethane (5.00 mL) were added bis(trichloromethyl) carbonate (195 mg, 658 umol, 1.60 eq) and N,N-diisopropylethylamine (106 mg, 822 umol, 143 uL, 2.00 eq) at 25° C. The mixture was stirred at 25° C. for 1 h. The reaction mixture was filtered and the filtrate was concentrated under reduced pressure to afford (cyclopropylmethyl)(methyl)carbamic chloride (60.0 mg, crude) as a yellow solid.

### Step 2. Procedure for Preparation of Compound 3—azetidin-3-yl (1-(4-(2,6-dioxopiperidin-3-yl)-3,5-difluorophenyl)azetidin-3-yl)carbamate

To a solution of tert-butyl 3-(((1-(4-(2,6-dioxopiperidin-3-yl)-3,5-difluorophenyl)azetidin-3-yl)carbamoyl)oxy)azetidine-1-carboxylate (250 mg, 506 umol, 1.00 eq) in dichloromethane (4.00 mL) was added trifluoroacetic acid (1.54 g, 13.5 mmol, 1.00 mL, 26.7 eq). The mixture was stirred at 25° C. for 2 h. The reaction mixture was concentrated under reduced pressure to afford azetidin-3-yl (1-(4-(2,6-dioxopiperidin-3-yl)-3,5-difluorophenyl)azetidin-3-yl)carbamate (190 mg, 482 umol, 95% yield) as a yellow solid. MS (ESI) m/z 395.0 [M+H]⁺

### Step 3. Procedure for Preparation of 1-((cyclopropylmethyl)(methyl)carbamoyl)azetidin-3-yl (1-(4-(2,6-dioxopiperidin-3-yl)-3,5-difluorophenyl)azetidin-3-yl)carbamate

To a solution of azetidin-3-yl (1-(4-(2,6-dioxopiperidin-3-yl)-3,5-difluorophenyl)azetidin-3-yl)carbamate (159 mg, 403 umol, 1.00 eq) in dichloromethane (5.00 mL) were added N,N-diisopropylethylamine (156 mg, 1.21 mmol, 211 uL, 3.00 eq) and (cyclopropylmethyl)(methyl)carbamic chloride (53.6 mg, 363 umol, 0.900 eq). The mixture was stirred at 25° C. for 2 h. The reaction mixture was concentrated under reduced pressure to give a residue. The residue was purified by Prep-HPLC (column: Phenomenex luna C18 150*25 mm*10 um; mobile phase: [water (formic acid)-acetonitrile]; B %: 29%-59%, 9 min) and lyophilized to afford 1-((cyclopropylmethyl)(methyl)carbamoyl)azetidin-3-yl (1-(4-(2,6-dioxopiperidin-3-yl)-3,5-difluorophenyl)azetidin-3-yl)carbamate (30.53 mg, 59.79 umol, 14.83% yield, 99% purity) as a white solid.

¹H NMR (400 MHz, DMSO-d₆) δ=10.86 (s, 1H), 8.10 (br d, J=7.5 Hz, 1H), 6.15 (d, J=11.0 Hz, 2H), 5.09-4.94 (m, 1H), 4.48-4.37 (m, 1H), 4.18-4.00 (m, 5H), 3.75 (br dd, J=4.0, 9.5 Hz, 2H), 3.64 (br t, J=6.7 Hz, 2H), 3.00 (d, J=6.8 Hz, 2H), 2.80 (s, 3H), 2.75 (br dd, J=5.2, 12.9 Hz, 1H), 2.53-2.51 (m, 1H), 2.07 (br dd, J=3.5, 13.3 Hz, 1H), 1.99-1.84 (m, 1H), 0.94-0.85 (m, 1H), 0.50-0.33 (m, 2H), 0.23-0.13 (m, 2H). MS (ESI) m/z 506.1 [M+H]⁺

### Example 44. Synthesis of Compound 44

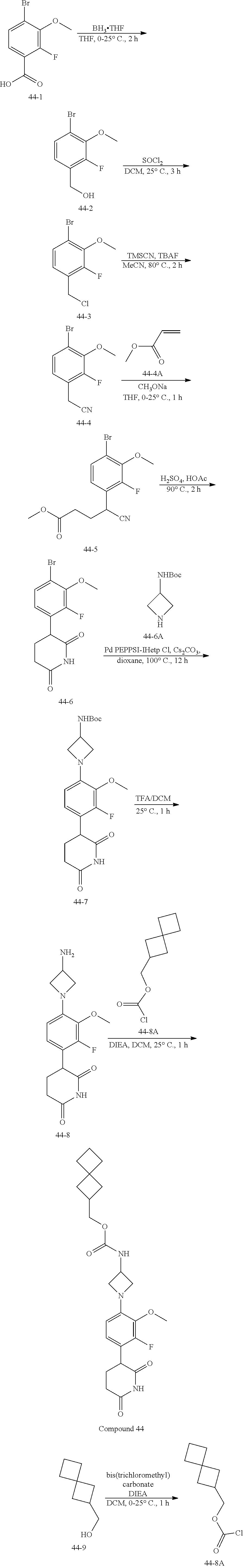

### Step 1. Procedure for Preparation of Compound 8A—spiro[3.3]heptan-2-ylmethyl carbonochloridate

To a solution of spiro[3.3]heptan-2-ylmethanol (60.0 mg, 475 umol, 1.00 eq) in dichloromethane (2.00 mL) were added bis(trichloromethyl) carbonate (226 mg, 761 umol, 1.60 eq) and N,N-diisopropylethylamine (123 mg, 951 umol, 166 uL, 2.00 eq) at 0° C. The mixture was stirred at 25° C. for 1 h. The reaction mixture was concentrated under reduced pressure to afford spiro[3.3]heptan-2-ylmethyl carbonochloridate (80 mg, crude) as a yellow solid.

### Step 2. Procedure for Preparation of Compound 2—(4-bromo-2-fluoro-3-methoxyphenyl)methanol

To a solution of 4-bromo-2-fluoro-3-methoxybenzoic acid (3.70 g, 14.9 mmol, 1.00 eq) in tetrahydrofuran (20.0 mL) was dropwise added borane tetrahydrofuran complex (1.00 M, 59.4 mL, 4.00 eq) under nitrogen atmosphere at 0° C. After addition, the mixture was stirred at 25° C. for 2 h. The reaction mixture was quenched by addition methanol (20.0 mL) slowly at 25° C. and then diluted with water (10.0 mL). The mixture was extracted with ethyl acetate (3×30 mL). The combined organic layers were washed with brine (30 mL), dried over sodium sulfate, filtered and concentrated under reduced pressure to give a residue. The residue was purified by column chromatography (SiO₂, petroleum ether/ethyl acetate=5/1) to afford 4-bromo-2-fluoro-3-methoxyphenyl)methanol (3.00 g, 12.8 mmol, 86% yield) as colorless oil.

¹H NMR (400 MHz, DMSO-d₆) δ=7.44 (dd, J=1.5, 8.4 Hz, 1H), 7.15 (t, J=7.8 Hz, 1H), 5.37 (t, J=5.8 Hz, 1H), 4.52 (d, J=5.5 Hz, 2H), 3.86 (d, J=0.8 Hz, 3H).

### Step 3. Procedure for Preparation of Compound 3—1-bromo-4-(chloromethyl)-3-fluoro-2-methoxybenzene

To a solution of (4-bromo-2-fluoro-3-methoxy-phenyl)methanol (3.00 g, 12.8 mmol, 1.00 eq) in dichloromethane (30.0 mL) was added thionyl chloride (3.04 g, 25.5 mmol, 1.85 mL, 2.00 eq). The mixture was stirred at 25° C. for 3 h. The reaction mixture was concentrated under reduced pressure to give a residue. The residue was purified by column chromatography (SiO₂, petroleum ether/ethyl acetate=50/1) to afford 1-bromo-4-(chloromethyl)-3-fluoro-2-methoxy-benzene (2.60 g, 10.3 mmol, 80% yield) as colorless oil.

¹H NMR (400 MHz, CDCl₃-d) δ=7.25 (dd, J=1.9, 8.4 Hz, 1H), 7.03-6.89 (m, 1H), 4.52 (d, J=1.3 Hz, 2H), 3.90 (d, J=1.4 Hz, 3H).

### Step 4. Procedure for Preparation of Compound 4—2-(4-bromo-2-fluoro-3-methoxyphenyl)acetonitrile

To a solution of 1-bromo-4-(chloromethyl)-3-fluoro-2-methoxybenzene (2.60 g, 103 mmol, 1.00 eq) in acetonitrile (40.0 mL) were added trimethylsilanecarbonitrile (3.05 g, 30.8 mmol, 3.85 mL, 3.00 eq) and tetrabutylammonium fluoride (1.00 M, 30.77 mL, 3.00 eq). The mixture was stirred at 80° C. for 2 h. The reaction mixture was quenched by addition water (10 mL), and then extracted with ethyl acetate (3×10 mL). The combined organic layers were filtered and concentrated under reduced pressure to give a residue. The residue was purified by column chromatography (SiO₂, petroleum ether/ethyl acetate=50/1) to afford 2-(4-bromo-2-fluoro-3-methoxyphenyl)acetonitrile (2.00 g, 8.19 mmol, 80% yield) as a white solid.

¹H NMR (400 MHz, CDCl₃-d) δ=7.29 (dd, J=1.0, 8.4 Hz, 1H), 6.97 (t, J=7.8 Hz, 1H), 3.90 (s, 3H), 3.65 (s, 2H).

### Step 5. Procedure for Preparation of Compound 5—methyl 4-(4-bromo-2-fluoro-3-methoxyphenyl)-4-cyanobutanoate

To a solution of 2-(4-bromo-2-fluoro-3-methoxyphenyl)acetonitrile (500 mg, 2.05 mmol, 1.00 eq) in tetrahydrofuran (6.00 mL) was added sodium methylate (16.6 mg, 307 umol, 0.150 eq) at 0° C., and then methyl acrylate (194 mg, 2.25 mmol, 203 uL, 1.10 eq) was added into the mixture at 0° C. The mixture was stirred at 25° C. for 1 h. The reaction mixture was diluted by addition water (10 mL) and extracted with ethyl acetate (3×20 mL). The combined organic layers were dried over sodium sulfate, filtered and concentrated under reduced pressure to give a residue. The residue was purified by column chromatography (SiO₂, petroleum ether/ethyl acetate=50/1 to 20/1) to afford methyl 4-(4-bromo-2-fluoro-3-methoxyphenyl)-4-cyanobutanoate (600 mg, 1.82 mmol, 89% yield) as a white solid.

¹H NMR (400 MHz, CDCl₃-d) δ=7.31 (dd, J=1.9, 8.5 Hz, 1H), 6.98 (dd, J=7.1, 8.4 Hz, 1H), 4.24-4.11 (m, 1H), 3.90 (d, J=1.4 Hz, 3H), 2.45 (q, J=7.4 Hz, 2H), 2.16 (dq, J=2.3, 7.3 Hz, 2H)

### Step 6. Procedure for Preparation of Compound 6—3-(4-bromo-2-fluoro-3-methoxyphenyl)piperidine-2,6-dione

To a solution of methyl 4-(4-bromo-2-fluoro-3-methoxyphenyl)-4-cyanobutanoate (500 mg, 1.51 mmol, 1.00 eq) in acetic acid (5.00 mL) was added sulfuric acid (920 mg, 9.38 mmol, 0.500 mL, 6.19 eq). The mixture was stirred at 90° C. for 2 h. The reaction mixture was diluted with water (10 mL) and filtered. The filter cake was concentrated under reduced pressure to give a residue. The residue was triturated with petroleum ether (5 mL) and then filtered. The second filter cake was concentrated under reduced pressure to afford 3-(4-bromo-2-fluoro-3-methoxyphenyl)piperidine-2,6-dione (450 mg, 1.42 mmol, 94% yield) as a gray solid.

¹H NMR (400 MHz, DMSO-d₆) δ=10.92 (s, 1H), 7.44 (dd, J=1.6, 8.4 Hz, 1H), 7.09-6.95 (m, 1H), 4.10 (br dd, J=4.9, 12.7 Hz, 1H), 3.86 (s, 3H), 2.78-2.70 (m, 1H), 2.56-2.52 (m, 1H), 2.28-2.17 (m, 1H), 2.10-1.96 (m, 1H).

### Step 7. Procedure for Preparation of Compound 6—tert-butyl (1-(4-(2,6-dioxopiperidin-3-yl)-3-fluoro-2-methoxyphenyl)azetidin-3-yl)carbamate

A mixture of 3-(4-bromo-2-fluoro-3-methoxyphenyl)piperidine-2,6-dione (100 mg, 316 umol, 1.00 eq), tert-butyl azetidin-3-ylcarbamate (81.7 mg, 475 umol, 1.50 eq), cesium carbonate (309 mg, 949 umol, 3.00 eq), and [1,3-bis[2,6-bis(1-propylbutyl)phenyl]-4,5-dichloro-imidazol-2-ylidene]-dichloro-(3-chloropyridin-1-ium-1-yl)palladium (30.8 mg, 31.6 umol, 0.100 eq) in dioxane (3.00 mL) was degassed and purged with nitrogen atmosphere for 3 times, and then the mixture was stirred at 100° C. for 12 h under nitrogen atmosphere. The reaction mixture was filtered and the filtrate was concentrated under reduced pressure to give a residue. The residue was purified by column chromatography (SiO₂, petroleum ether/ethyl acetate=5/1 to 1/1) to afford tert-butyl (1-(4-(2,6-dioxopiperidin-3-yl)-3-fluoro-2-methoxyphenyl)azetidin-3-yl)carbamate (50.0 mg, 123 umol, 39% yield) as yellow oil. MS (ESI) m/z 407.9 [M+H]⁺

### Step 8. Procedure for Preparation of Compound 8—3-(4-(3-aminoazetidin-1-yl)-2-fluoro-3-methoxyphenyl)piperidine-2,6-dione

To a solution of tert-butyl (1-(4-(2,6-dioxopiperidin-3-yl)-3-fluoro-2-methoxyphenyl)azetidin-3-yl)carbamate (50.0 mg, 123 umol, 1.00 eq) in dichloromethane (4.00 mL) was added trifluoroacetic acid (1.54 g, 13.5 mmol, 1.00 mL, 110 eq). The mixture was stirred at 25° C. for 1 h. The reaction mixture was concentrated under reduced pressure to afford 3-(4-(3-aminoazetidin-1-yl)-2-fluoro-3-methoxyphenyl)piperidine-2,6-dione (30 mg, 97.6 umol, 80% yield) as a white solid. MS (ESI) m/z 308.0 [M+H]⁺

### Step 9. Procedure for Preparation of spiro[3.3]heptan-2-ylmethyl (1-(4-(2,6-dioxopiperidin-3-yl)-3-fluoro-2-methoxyphenyl)azetidin-3-yl)carbamate

To a solution of 3-(4-(3-aminoazetidin-1-yl)-2-fluoro-3-methoxyphenyl)piperidine-2,6-dione (30.0 mg, 97.6 umol, 1.00 eq) in dichloromethane (2.00 mL) were added N,N-diisopropylethylamine (37.9 mg, 293 umol, 51.0 uL, 3.00 eq) and spiro[3.3]heptan-2-ylmethyl carbonochloridate (18.4 mg, 97.6 umol, 1.00 eq). The mixture was stirred at 25° C. for 1 h. The reaction mixture was filtered and concentrated under reduced pressure to give a residue. The residue was purified by reverse phase chromatography (C18, 40 g; condition: water/acetonitrile=100:0 to 0:100, 0.1% formic acid) and lyophilized to afford spiro[3.3]heptan-2-ylmethyl (1-(4-(2,6-dioxopiperidin-3-yl)-3-fluoro-2-methoxyphenyl)azetidin-3-yl)carbamate (10.84 mg, 23.35 umol, 23.92% yield, 99% purity) as a white solid.

¹H NMR (400 MHz, DMSO-d₆) δ=10.81 (s, 1H), 7.77 (br d, J=7.4 Hz, 1H), 6.81 (t, J=8.1 Hz, 1H), 6.22 (d, J=8.4 Hz, 1H), 4.44-4.31 (m, 1H), 4.14 (t, J=7.6 Hz, 2H), 3.85 (m, 3H), 3.69 (s, 3H), 3.65 (t, J=7.1 Hz, 2H), 2.72-2.68 (m, 1H), 2.38-2.33 (m, 1H), 2.21-2.04 (m, 2H), 2.03-1.84 (m, 7H), 1.80-1.69 (m, 4H). MS (ESI) m/z 460.2 [M+H]⁺

### Example 45. Synthesis of Compound 45

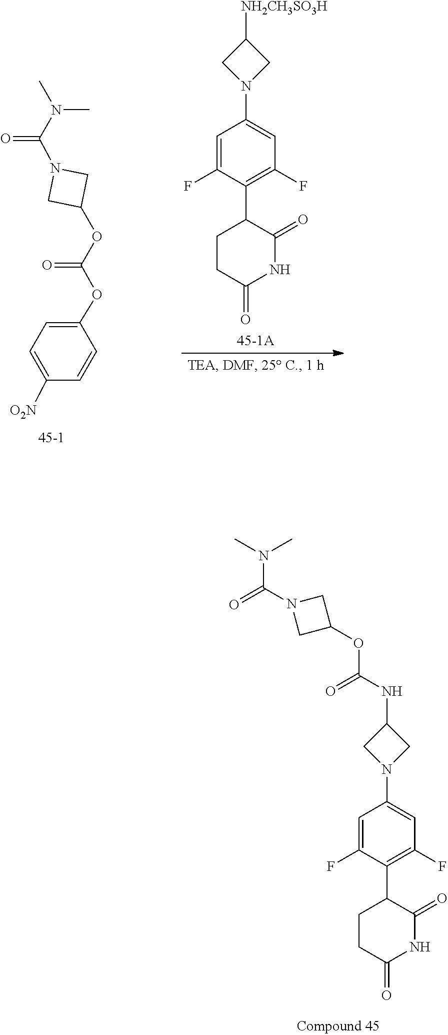

### Step 1. Procedure for Preparation of 1-(dimethylcarbamoyl)azetidin-3-yl (1-(4-(2,6-dioxopiperidin-3-yl)-3,5-difluorophenyl)azetidin-3-yl)carbamate

To the solution of 1-(dimethylcarbamoyl)azetidin-3-yl (4-nitrophenyl) carbonate (50.0 mg, 113 umol, 70% purity, 1.00 eq), 3-(4-(3-aminoazetidin-1-yl)-2,6-difluorophenyl)piperidine-2,6-dione (44.3 mg, 113 umol, 1.00 eq, methanesulfonic acid) in dimethylformamide (0.500 mL) was added triethylamine (22.9 mg, 226 umol, 31.5 uL, 2.00 eq). Then the reaction was stirred at 25° C. for 1 h. The reaction mixture was concentrated under reduced pressure to give a residue. The residue was purified by Prep-HPLC (column: Phenomenex luna C18 150*25 mm*10 um; mobile phase: [water (formic acid)-acetonitrile]; B %: 21%-51%, 58 min) and lyophilized to afford 1-(dimethylcarbamoyl)azetidin-3-yl (1-(4-(2,6-dioxopiperidin-3-yl)-3,5-difluorophenyl)azetidin-3-yl)carbamate (20.40 mg, 42.51 umol, 37% yield, 97% purity) as a white solid.

¹H NMR (400 MHz, DMSO-d) δ=10.85 (br s, 1H), 8.10 (br d, J=6.4 Hz, 1H), 6.15 (br d, J=11.3 Hz, 2H), 5.00 (br s, 1H), 4.42 (br d, J=6.1 Hz, 1H), 4.20-4.07 (m, 4H), 4.07-4.01 (m, 1H), 3.76 (br d, J=5.3 Hz, 2H), 3.65 (br s, 2H), 2.75 (br s, 8H), 2.14-2.02 (m, 1H), 1.96 (br s, 1H). MS (ESI) m/z 466.1 [M+H]⁺

### Example 46. Synthesis of Compound 46

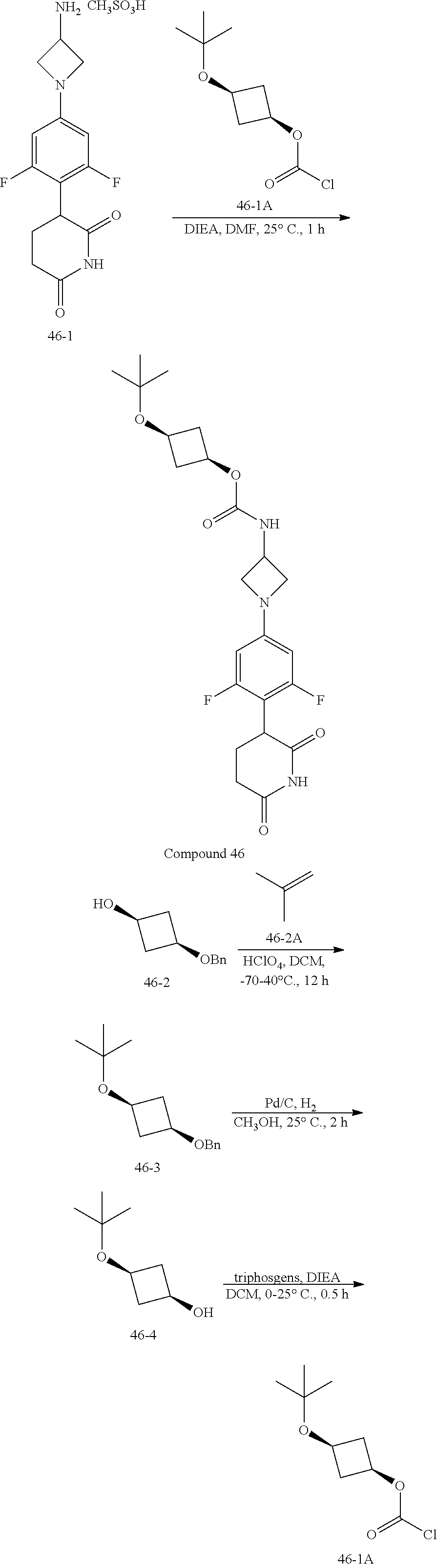

### Step 1. Procedure for Preparation of Compound 3 (((1s, 3s)-3-(tert-butoxy)cyclobutoxy)methyl)benzene

To the solution of (1s, 3s)-3-(benzyloxy)cyclobutanol (100 mg, 561 umol, 1.00 eq), perchloric acid (84.6 mg, 841 umol, 50.9 uL, 1.50 eq) in dichloromethane (2.00 mL) was added 2-methylprop-1-ene (2.40 M, 2.34 mL, 10.0 eq) at −70° C. Then the reaction was stirred at −40° C. for 12 h. The reaction was neutralized with saturated aqueous sodium carbonate (5 mL), then extracted by dichloromethane (10 mL). The organic layer was washed with brine (10 mL), and dried over anhydrous sodium sulfate, filtered and concentrated under reduced pressure to give a residue. The residue was purified by column chromatography (SiO₂, petroleum ether/ethyl acetate=20/1) to afford (((1s, 3s)-3-(tert-butoxy)cyclobutoxy)methyl)benzene (110 mg, 469 umol, 83% yield) as colorless oil.

¹H NMR (400 MHz, CDCl₃) δ=7.28-7.17 (m, 5H), 4.33 (s, 2H), 3.69-3.52 (m, 2H), 2.55-2.48 (m, 2H), 1.97-1.89 (m, 2H), 1.10 (s, 9H).

### Step 2. Procedure for Preparation of Compound 4—(1s, 3s)-3-(tert-butoxy)cyclobutanol

To a solution of (((1s, 3s)-3-(tert-butoxy)cyclobutoxy)methyl)benzene (110 mg, 469 umol, 1.00 eq) in methanol (5.00 mL) was added palladium on activated carbon (220 mg, 10% purity) under nitrogen atmosphere. Then the reaction was stirred under 15 psi of hydrogen atmosphere at 25° C. for 2 h. The suspension was filtered and the filter cake was washed with acetonitrile (10 mL). The combined filtrates were concentrated under reduced pressure to afford (1s, 3s)-3-(tert-butoxy)cyclobutanol (56.0 mg, 388 umol, 82% yield) as colorless oil.

¹H NMR (400 MHz, CDCl₃) δ=3.88 (quin, J=7.3 Hz, 1H), 3.72-3.63 (m, 1H), 2.73-2.65 (m, 2H), 1.94-1.87 (m, 2H), 1.83 (br s, 1H), 1.17 (s, 9H).

### Step 3. Procedure for Preparation of Compound 1A—(1s, 3s)-3-(tert-butoxy)cyclobutyl carbonochloridate

To the solution of (1s, 3s)-3-(tert-butoxy)cyclobutanol (56.0 mg, 388 umol, 1.00 eq) in dichloromethane (0.500 mL) were added bis(trichloromethyl) carbonate (115 mg, 388 umol, 1.00 eq) and N, N-diisopropylethylamine (150 mg, 1.16 mmol, 203 uL, 3.00 eq) at 0° C. Then the reaction was stirred at 25° C. for 0.5 h. The reaction mixture was concentrated under reduced pressure to afford (1s, 3s)-3-(tert-butoxy)cyclobutyl carbonochloridate (80.2 mg, crude) as an orange solid.

### Step 4. Procedure for Preparation of (1s, 3s)-3-(tert-butoxy)cyclobutyl (1-(4-(2,6-dioxopiperidin-3-yl)-3,5-difluorophenyl)azetidin-3-yl)carbamate

To the solution of (1s, 3s)-3-(tert-butoxy)cyclobutyl carbonochloridate (80.2 mg, 388 umol, 1.00 eq), 3-(4-(3-aminoazetidin-1-yl)-2,6-difluorophenyl)piperidine-2,6-dione (152 mg, 388 umol, 1.00 eq, methanesulfonic acid) in dichloromethane (0.50 mL) was added N, N-diisopropylethylamine (201 mg, 1.55 mmol, 270 uL, 4.00 eq). Then the reaction was stirred at 25° C. for 1 h. The reaction mixture was concentrated under reduced pressure to give a residue. The residue was purified by Prep-HPLC (column: Waters xbridge 150*25 mm 10 um; mobile phase: [water (formic acid)-acetonitrile]; B %: 32%-62%, 8 min) and lyophilized to afford (1s, 3s)-3-(tert-butoxy)cyclobutyl (1-(4-(2,6-dioxopiperidin-3-yl)-3,5-difluorophenyl)azetidin-3-yl)carbamate (42.44 mg, 82.14 umol, 21% yield, 99% purity, formate) as a yellow solid.

¹H NMR (400 MHz, DMSO-d) δ=10.86 (s, 1H), 8.39 (br s, 1H), 7.86 (br d, J=7.3 Hz, 1H), 6.15 (s, 1H), 6.13 (s, 1H), 4.50-4.43 (m, 1H), 4.42-4.35 (m, 1H), 4.07 (br t, J=7.8 Hz, 2H), 4.05-4.00 (m, 1H), 3.83-3.76 (m, 1H), 3.61 (br t, J=6.8 Hz, 2H), 2.82-2.73 (m, 1H), 2.64-2.58 (m, 2H), 2.47 (br s, 1H), 2.12-2.02 (m, 1H), 1.98-1.91 (m, 1H), 1.85-1.77 (m, 2H), 1.10 (s, 9H). MS (ESI) m/z 466.2 [M+H]⁺

### Example 47. Synthesis of Compound 47

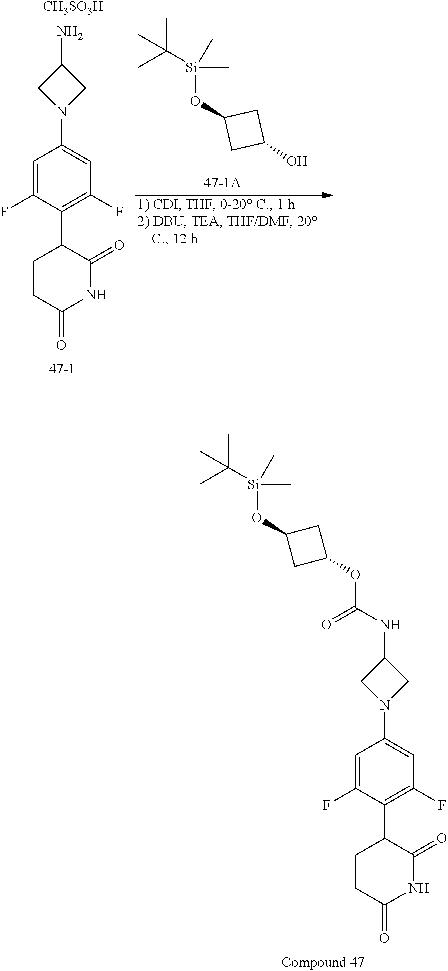 [SMILES: NC1CN(c2cc(F)c(C3CCC(=O)NC3=O)c(F)c2)C1 ; "CH3SO3H" ; CC(C)(C)[Si](C)(C)O[C@H]1C[C@H](O)C1 ; CC(C)(C)[Si](C)(C)O[C@H]1C[C@H](OC(=O)NC2CN(c3cc(F)c(C4CCC(=O)NC4=O)c(F)c3)C2)C1]

### Step 1. Procedure for (1r, 3r)-3-((tert-butyldimethylsilyl)oxy)cyclobutyl (1-(4-(2,6-dioxopiperidin-3-yl)-3,5-difluorophenyl)azetidin-3-yl)carbamate

To a solution of (1r, 3r)-3-((tert-butyldimethylsilyl)oxy)cyclobutanol (50.0 mg, 247 umol, 1.00 eq) in tetrahydrofuran (1.00 mL) was added di(1H-imidazol-1-yl)methanone (40.1 mg, 247 umol, 1.00 eq) at 0° C. The reaction mixture was stirred at 20° C. for 1 h. The resulting solution was added to a solution of 3-(4-(3-aminoazetidin-1-yl)-2,6-difluorophenyl)piperidine-2,6-dione (97.0 mg, 248 umol, 1.00 eq, methanesulfonic acid), triethylamine (25.1 mg, 248 umol, 34.5 uL, 1.00 eq) and 2,3,4,6,7,8,9,10-octahydropyrimido[1,2-a]azepine (37.7 mg, 248 umol, 37.4 uL, 1.00 eq) in tetrahydrofuran (1.00 mL) and dimethylformamide (1.00 mL). The reaction mixture was stirred at 20° C. for 12 h. The reaction mixture was concentrated under reduced pressure to give a residue. The residue was purified by column chromatography (SiO₂, petroleum ether/ethyl acetate=2/1) to afford (1r, 3r)-3-((tert-butyldimethylsilyl)oxy)cyclobutyl(1-(4-(2,6-dioxopiperidin-3-yl)-3,5-difluorophenyl)azetidin-3-yl)carbamate (29.29 mg, 52.58 umol, 21% yield, 94% purity) as a white solid.

¹H NMR (400 MHz, DMSO-d₆) δ=10.83 (s, 1H), 7.86 (br d, J=7.3 Hz, 1H), 6.12 (d, J=11.1 Hz, 2H), 4.89 (td, J=3.5, 6.8 Hz, 1H), 4.46 (t, J=6.2 Hz, 1H), 4.41-4.32 (m, 1H), 4.11-3.97 (m, 3H), 3.60 (t, J=6.8 Hz, 2H), 2.82-2.70 (m, 1H), 2.45 (br s, 1H), 2.32-2.22 (m, 2H), 2.21-2.13 (m, 2H), 2.05 (dq, J=3.5, 13.0 Hz, 1H), 1.96-1.84 (m, 1H), 0.83 (s, 9H), 0.00 (s, 6H). MS (ESI) m/z 524.0 [M+H]⁺

### Example 48. Synthesis of Compound 48

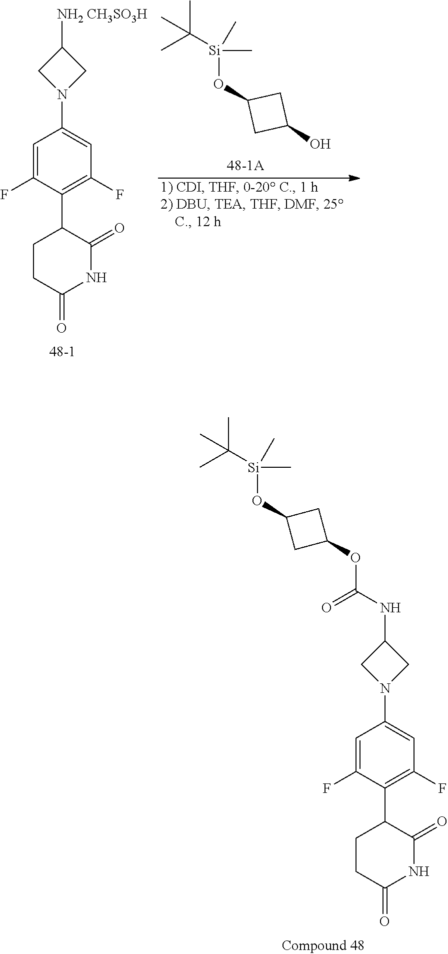

### Step 1. Procedure for Preparation of (1s, 3s)-3-((isopropyldimethylsilyl)oxy)cyclobutyl (1-(4-(2,6-dioxopiperidin-3-yl)-3,5-difluorophenyl)azetidin-3-yl)carbamate

To the solution of (1s, 3s)-3-((tert-butyldimethylsilyl)oxy)cyclobutanol (50.0 mg, 247 umol, 1.00 eq) in tetrahydrofuran (0.500 mL) was added di(1H-imidazol-1-yl)methanone (40.1 mg, 247 umol, 1.00 eq) at 0° C. Then the reaction was stirred at 25° C. for 0.5 h. Then to the mixture were added dimethylformamide (0.500 mL), 3-(4-(3-aminoazetidin-1-yl)-2,6-difluorophenyl)piperidine-2,6-dione (100 mg, 255 umol, 1.00 eq, methanesulfonic acid), triethylamine (25.9 mg, 255 umol, 35.6 uL, 1.00 eq), 2,3,4,6,7,8,9,10-octahydropyrimido[1,2-a]azepine (38.9 mg, 255 umol, 38.5 uL, 1.00 eq). Then the reaction was stirred at 25° C. for 12 h. The reaction mixture was concentrated under reduced pressure to give a residue. The residue was purified by column chromatography (SiO₂, petroleum ether/ethyl acetate=2/1) to afford (1s, 3s)-3-((isopropyldimethylsilyl)oxy)cyclobutyl (1-(4-(2,6-dioxopiperidin-3-yl)-3,5-difluorophenyl)azetidin-3-yl)carbamate (47.32 mg, 89.46 umol, 35% yield, 99% purity) as a white solid.

¹H NMR (400 MHz, DMSO-d) δ=10.85 (s, 1H), 7.86 (br d, J=7.4 Hz, 1H), 6.15 (s, 1H), 6.13 (s, 1H), 4.47-4.36 (m, 2H), 4.08 (br t, J=7.8 Hz, 2H), 4.05-3.92 (m, 2H), 3.61 (br t, J=6.8 Hz, 2H), 2.83-2.74 (m, 1H), 2.67 (tdd, J=3.3, 6.3, 9.3 Hz, 2H), 2.49-2.46 (m, 1H), 2.11-2.01 (m, 1H), 1.96-1.90 (m, 1H), 1.87-1.78 (m, 2H), 0.85 (s, 9H), 0.02 (s, 6H). MS (ESI) m/z 524.2 [M+H]⁺

### Example 49. Synthesis of Compound 49

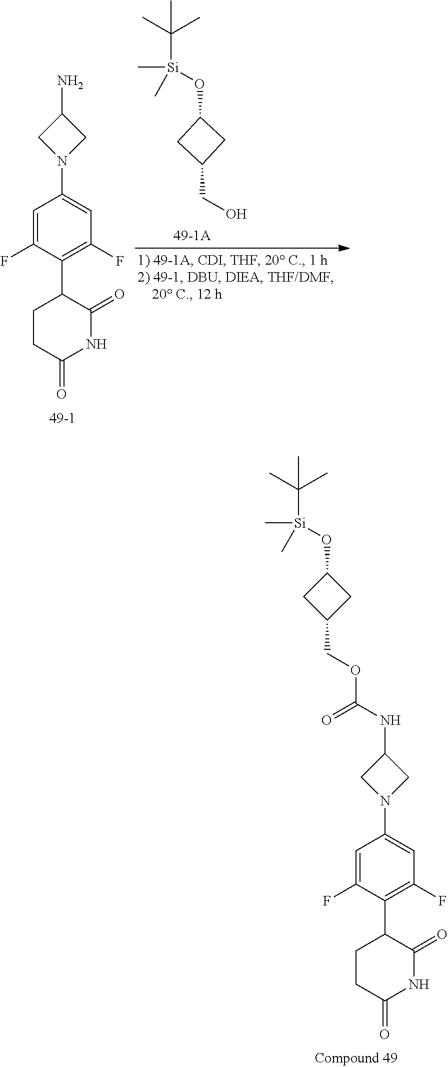 [SMILES: NC1CN(c2cc(F)c(C3CCC(=O)NC3=O)c(F)c2)C1 ; CC(C)(C)[Si](C)(C)O[C@H]1C[C@@H](CO)C1 ; CC(C)(C)[Si](C)(C)O[C@H]1C[C@@H](COC(=O)NC2CN(c3cc(F)c(C4CCC(=O)NC4=O)c(F)c3)C2)C1]

### Step 1. Procedure for Preparation of ((1s, 3s)-3-((tert-butyldimethylsilyl)oxy)cyclobutyl)methyl (1-(4-(2,6-dioxopiperidin-3-yl)-3,5-difluorophenyl)azetidin-3-yl)carbamate

To a solution of ((1s, 3s)-3-((tert-butyldimethylsilyl)oxy)cyclobutyl)methanol (50.0 mg, 231 umol, 1.00 eq) in tetrahydrofuran (0.500 mL) was added di(1H-imidazol-1-yl)methanone (56.2 mg, 347 umol, 1.50 eq). The mixture was stirred at 20° C. for 1 h. The resulting solution was added to a mixture of 3-(4-(3-aminoazetidin-1-yl)-2,6-difluorophenyl)piperidine-2,6-dione (85.0 mg, 217 umol, 1.00 eq, methanesulfonic acid), 2,3,4,6,7,8,9,10-octahydropyrimido[1,2-a]azepine (33.1 mg, 217 umol, 32.7 uL, 1.00 eq) and triethylamine (22.0 mg, 217 umol, 30.2 uL, 1.00 eq) in tetrahydrofuran (0.500 mL) and dimethylformamide (1.00 mL). The reaction mixture was stirred at 20° C. for 12 h. The reaction mixture was concentrated under reduced pressure to give a residue. The residue was dissolved in dimethylformamide (0.5 mL) and then filtered. The filtrate was purified by reverse phase chromatography (C18, 40 g; condition: water/acetonitrile=100:0 to 18:82) and lyophilized to afford ((1s, 3s)-3-((tert-butyldimethylsilyl)oxy)cyclobutyl)methyl (1-(4-(2,6-dioxopiperidin-3-yl)-3,5-difluorophenyl)azetidin-3-yl)carbamate (11.08 mg, 19.58 umol, 9% yield, 95% purity) as a white solid.

¹H NMR (400 MHz, DMSO-d₆) δ=10.83 (s, 1H), 7.83 (br d, J=6.1 Hz, 1H), 6.14 (s, 1H), 6.12 (s, 1H), 4.47-4.31 (m, 1H), 4.15-4.00 (m, 4H), 3.93 (d, J=6.1 Hz, 2H), 3.67-3.59 (m, 2H), 2.83-2.71 (m, 1H), 2.52 (br s, 1H), 2.29-2.24 (m, 2H), 2.07 (br dd, J=3.9, 13.3 Hz, 1H), 1.99-1.91 (m, 2H), 1.65-1.52 (m, 2H), 0.85 (s, 9H), 0.06-0.05 (m, 6H). MS (ESI) m/z 538.3 [M+H]⁺

### Example 50. Synthesis of Compound 50

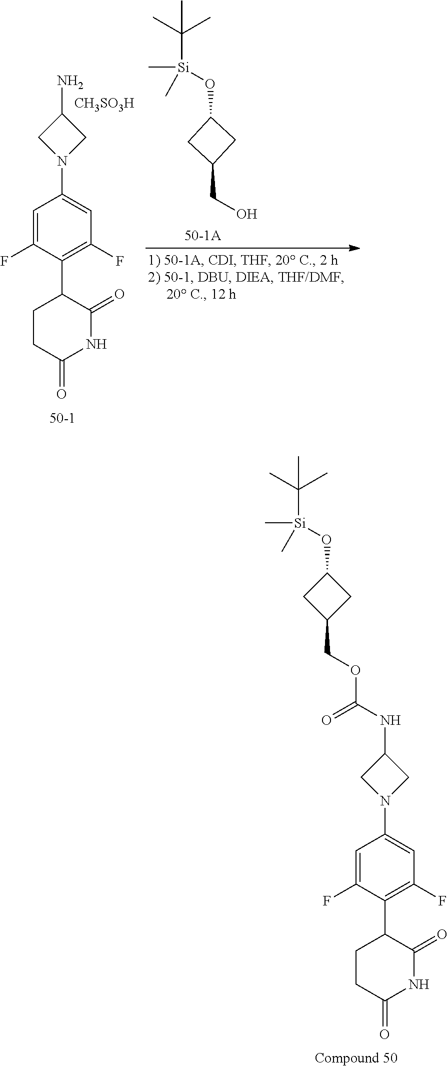

### Step 1. Procedure for Preparation of ((1r, 3r)-3-((tert-butyldimethylsilyl)oxy) cyclobutyl)methyl (1-(4-(2,6-dioxopiperidin-3-yl)-3,5-difluorophenyl)azetidin-3-yl)carbamate

To a solution of ((1r, 3r)-3-((tert-butyldimethylsilyl)oxy)cyclobutyl)methanol (50.0 mg, 231 umol, 1.00 eq) in tetrahydrofuran (0.500 mL) was added di(1H-imidazol-1-yl)methanone (37.5 mg, 231 umol, 1.00 eq) at 0° C., the mixture was stirred at 20° C. for 2 h. The resulting solution was added to a mixture of 3-(4-(3-aminoazetidin-1-yl)-2,6-difluorophenyl)piperidine-2,6-dione (88.3 mg, 225 umol, 1.00 eq, methanesulfonic acid), 2,3,4,6,7,8,9,10-octahydropyrimido[1,2-a]azepine (34.3 mg, 225 umol, 34.0 uL, 1.00 eq) and N,N-diisopropylethylamine (58.3 mg, 451 umol, 78.6 uL, 2.00 eq) in tetrahydrofuran (1.00 mL) and dimethylformamide (1.00 mL). The reaction mixture was stirred at 20° C. for 12 h. The reaction mixture was concentrated under reduced pressure to give a residue. The residue was diluted with dimethylformamide (1.00 mL) and then filtered. The filtrate was purified by Prep-HPLC (column: Phenomenex luna C18 150*25 mm*10 um; mobile phase: [water (ammonium bicarbonate)-acetonitrile]; B %: 53%-83%, 8 min) and lyophilized to afford ((1r, 3r)-3-((tert-butyldimethylsilyl)oxy)cyclobutyl)methyl (1-(4-(2,6-dioxopiperidin-3-yl)-3,5-difluorophenyl)azetidin-3-yl)carbamate (9.35 mg, 16.69 umol, 7% yield, 96% purity) as a white solid.

¹H NMR (400 MHz, DMSO-d₆) δ=10.85 (br s, 1H), 7.86 (br d, J=7.6 Hz, 1H), 6.15 (s, 1H), 6.13 (s, 1H), 4.45-4.36 (m, 2H), 4.09 (t, J=7.8 Hz, 2H), 4.03 (br dd, J=5.7, 13.7 Hz, 1H), 3.98 (d, J=7.3 Hz, 2H), 3.62 (br t, J=6.7 Hz, 2H), 2.82-2.73 (m, 1H), 2.52 (br s, 1H), 2.32-2.27 (m, 1H), 2.07 (br d, J=4.6 Hz, 1H), 2.06-2.01 (m, 2H), 2.01-1.97 (m, 1H), 1.97-1.88 (m, 2H), 0.84 (s, 9H), 0.01 (s, 6H). MS (ESI) m/z 538.6 [M+H]⁺

### Example 51. Synthesis of Compound 51

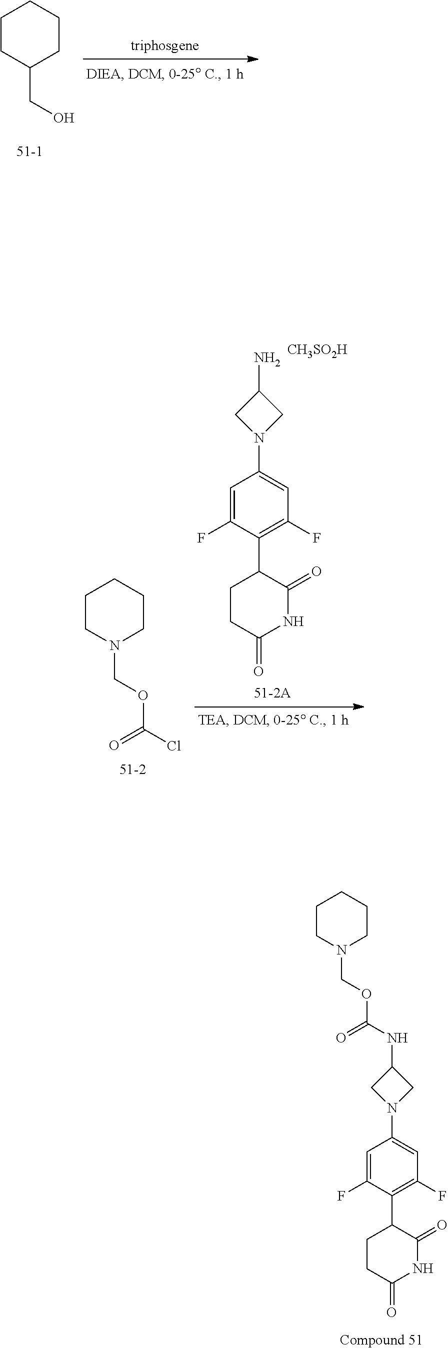

### Step 1. Procedure for Preparation of Compound 2—cyclohexylmethyl carbonochloridate

To a solution of cyclohexylmethanol (45.0 mg, 394 umol, 48.4 uL, 1.00 eq) in dichloromethane (1.00 mL) were added bis(trichloromethyl) carbonate (187 mg, 631 umol, 1.60 eq) and N,N-diisopropylethylamine (102 mg, 788 umol, 137 uL, 2.00 eq) at 0° C. The reaction mixture was stirred at 25° C. for 1 h. The reaction mixture was concentrated under reduced pressure to afford cyclohexylmethyl carbonochloridate (69.0 mg, 391 umol, 99% yield) as yellow oil.

### Step 2. Procedure for Preparation of cyclohexylmethyl (1-(4-(2,6-dioxopiperidin-3-yl)-3,5-difluorophenyl)azetidin-3-yl)carbamate

To a solution of 3-(4-(3-aminoazetidin-1-yl)-2,6-difluorophenyl)piperidine-2,6-dione (100 mg, 256 umol, 1.00 eq, methanesulfonic acid) in tetrahydrofuran (2.00 mL) were added N,N-diisopropylethylamine (66.0 mg, 511 umol, 89.0 uL, 2.00 eq) and cyclohexylmethyl carbonochloridate (67.7 mg, 383 umol, 1.50 eq). The mixture was stirred at 25° C. for 1 h. The reaction mixture was filtered and concentrated under reduced pressure to give a residue. The residue was purified by Prep-HPLC (column: Phenomenex luna C18 150*25 mm*10 um; mobile phase: [water (formic acid)-acetonitrile]; B %: 46%-76%, 15 min) and lyophilized to afford cyclohexylmethyl (1-(4-(2,6-dioxopiperidin-3-yl)-3,5-difluorophenyl)azetidin-3-yl)carbamate (13.07 mg, 29.11 umol, 11% yield, 97% purity) as an off-white solid.

¹H NMR (400 MHz, DMSO-d₆) δ=10.85 (s, 1H), 7.94-7.66 (m, 1H), 6.14 (d, J=11.1 Hz, 2H), 4.47-4.32 (m, 1H), 4.13-3.97 (m, 3H), 3.78 (d, J=6.5 Hz, 2H), 3.63 (t, J=6.8 Hz, 2H), 2.84-2.70 (m, 1H), 2.11-2.02 (m, 1H), 2.00-1.86 (m, 1H), 1.67 (br d, J=9.8 Hz, 4H), 1.61 (br d, J=3.6 Hz, 1H), 1.57-1.49 (m, 1H), 1.29-1.05 (m, 4H), 0.99-0.87 (m, 2H). MS (ESI) m/z 457.9 [M+Na]⁺

### Example 52. Synthesis of Compound 52

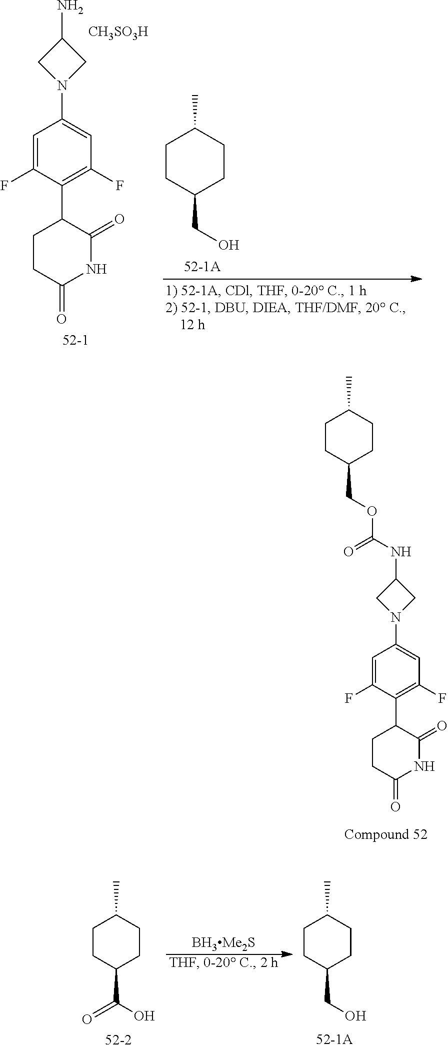

### Step 1. Procedure for Preparation of Compound 2—((1r, 4r)-4-methylcyclohexyl)methanol

To a solution of (1r, 4r)-4-methylcyclohexanecarboxylic acid (100 mg, 703 umol, 1.00 eq) in tetrahydrofuran (1.00 mL) was added borane dimethyl sulfide complex (10.0 M, 141 μL, 2.00 eq) at 0° C. The reaction mixture was stirred at 20° C. for 2 h. The reaction mixture was quenched by methanol (20.0 mL) and concentrated under reduced pressure to afford ((1r, 4r)-4-methylcyclohexyl)methanol (80.0 mg, 624 umol, 89% yield) as colorless oil.

¹H NMR (400 MHz, CDCl₃-d) δ=3.46 (d, J=6.4 Hz, 2H), 1.80-1.75 (m, 2H), 1.75-1.68 (m, 2H), 1.44-1.39 (m, 2H), 1.36-1.27 (m, 1H), 1.02-0.95 (m, 2H), 0.95-0.91 (m, 2H), 0.89 (d, J=6.6 Hz, 3H).

### Step 2. Procedure for Preparation of ((1r, 4r)-4-methylcyclohexyl)methyl (1-(4-(2,6-dioxopiperidin-3-yl)-3,5-difluorophenyl)azetidin-3-yl)carbamate

To a solution of ((1r, 4r)-4-methylcyclohexyl)methanol (35.0 mg, 273 umol, 1.00 eq) in tetrahydrofuran (1.00 mL) was added di(1H-imidazol-1-yl)methanone (44.0 mg, 273 umol, 1.00 eq) at 0° C., the mixture was stirred at 20° C. for 1 h. The resulting solution was added to a mixture of 3-(4-(3-aminoazetidin-1-yl)-2,6-difluorophenyl)piperidine-2,6-dione (106 mg, 270 umol, 1.00 eq, methanesulfonic acid), 2,3,4,6,7,8,9,10-octahydropyrimido[1,2-a]azepine (41.1 mg, 270 umol, 40.7 uL, 1.00 eq) and N,N-diisopropylethylamine (69.8 mg, 540 umol, 94.0 uL, 2.00 eq) in tetrahydrofuran (1.00 mL) and dimethylformamide (1.00 mL). The reaction mixture was stirred at 20° C. for 12 h. The reaction mixture was concentrated under reduced pressure to give a residue. The residue was diluted with dimethylformamide (1.00 mL) and then filtered. The filtrate was purified by Prep-HPLC (column: Phenomenex luna C18 150*25 mm*10 um; mobile phase: [water (formic acid)-acetonitrile]; B %: 52%-82%, 9 min) and lyophilized to afford ((1r, 4r)-4-methylcyclohexyl)methyl (1-(4-(2,6-dioxopiperidin-3-yl)-3,5-difluorophenyl) azetidin-3-yl)carbamate (13.95 mg, 28.86 umol, 11% yield, 93% purity) as a white solid.

¹H NMR (400 MHz, DMSO-d₆) δ=10.85 (s, 1H), 7.80 (br d, J=7.3 Hz, 1H), 6.15 (s, 1H), 6.13 (s, 1H), 4.45-4.35 (m, 1H), 4.08 (t, J=7.6 Hz, 2H), 4.03 (br dd, J=5.4, 12.8 Hz, 1H), 3.78 (d, J=6.5 Hz, 2H), 3.62 (t, J=6.8 Hz, 2H), 2.83-2.72 (m, 1H), 2.52 (br s, 1H), 2.11-2.03 (m, 1H), 1.96-1.90 (m, 1H), 1.67 (br d, J=9.4 Hz, 4H), 1.51-1.43 (m, 1H), 1.30-1.23 (m, 1H), 1.00-0.87 (m, 4H), 0.85 (d, J=6.5 Hz, 3H). MS (ESI) m/z 450.1 [M+H]⁺

### Example 53. Synthesis of Compound 53

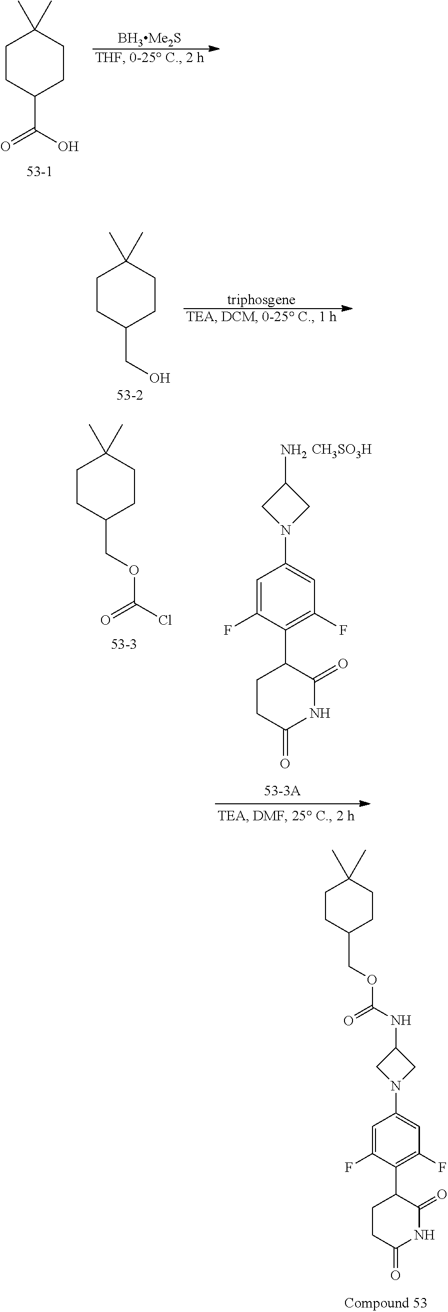

### Step 1. Procedure for Compound 2—(4,4-dimethylcyclohexyl)methanol

To a solution of 4,4-dimethylcyclohexanecarboxylic acid (300 mg, 1.92 mmol, 1.00 eq) in tetrahydrofuran (3.00 mL) was added borane dimethyl sulfide complex (10.0 M, 384 uL, 2.00 eq) at 0° C. The reaction mixture was stirred at 25° C. for 2 h. The mixture was quenched with methanol (10 mL) and concentrated under reduced pressure to afford (4,4-dimethylcyclohexyl)methanol (265 mg, 1.86 mmol, 97% yield) as colorless oil.

¹H NMR (400 MHz, CDCl₃-d) δ=3.48 (d, J=6.3 Hz, 2H), 1.64 (br s, 1H), 1.60-1.55 (m, 2H), 1.43-1.36 (m, 3H), 1.22-1.09 (m, 4H), 0.91 (s, 3H), 0.88 (s, 3H).

### Step 2. Procedure for Compound 3—(4,4-dimethylcyclohexyl)methyl carbonochloridate

To a solution of (4,4-dimethylcyclohexyl)methanol (50.0 mg, 351 umol, 1.00 eq) in dichloromethane (0.500 mL) were added triethylamine (71.1 mg, 703 umol, 97.8 uL, 2.00 eq) and triphosgene (104 mg, 351 umol, 1.00 eq) at 0° C. The reaction mixture was stirred at 25° C. for 1 h. The reaction mixture was concentrated under reduced pressure to afford (4,4-dimethylcyclohexyl)methyl carbonochloridate (71.9 mg, crude) as a white solid.

### Step 3. Procedure for (4,4-dimethylcyclohexyl)methyl (1-(4-(2,6-dioxopiperidin-3-yl)-3,5-difluorophenyl)azetidin-3-yl)carbamate

To a solution of 3-(4-(3-aminoazetidin-1-yl)-2,6-difluorophenyl)piperidine-2,6-dione (65.0 mg, 166 umol, 1.00 eq, methanesulfonic acid) and (4,4-dimethylcyclohexyl)methyl carbonochloridate (67.9 mg, 332 umol, 2.00 eq) in N,N-dimethyl formamide (1.00 mL) were added triethylamine (50.4 mg, 498 umol, 69.3 uL, 3.00 eq). The reaction mixture was stirred at 25° C. for 2 h. The reaction mixture was dissolved in N,N-dimethyl formamide (0.5 mL) and then filtered. The filtrate was purified by Prep-HPLC (column: Phenomenex luna C18 150*25 mm*10 um; mobile phase: [water (formic acid)-acetonitrile]; B %: 52%-82%, 9 min) and lyophilized to afford (4,4-dimethylcyclohexyl)methyl (1-(4-(2,6-dioxopiperidin-3-yl)-3,5-difluorophenyl)azetidin-3-yl)carbamate (23.55 mg, 50.30 umol, 30% yield, 99% purity) as a white solid.

¹H NMR (400 MHz, DMSO-d₆) δ=10.85 (s, 1H), 7.81 (br d, J=7.1 Hz, 1H), 6.14 (d, J=11.0 Hz, 2H), 4.46-4.35 (m, 1H), 4.08 (t, J=7.6 Hz, 2H), 4.03 (br dd, J=5.1, 12.6 Hz, 1H), 3.82 (br d, J=5.9 Hz, 2H), 3.63 (t, J=6.8 Hz, 2H), 2.83-2.72 (m, 1H), 2.53-2.51 (m, 1H), 2.07 (dq, J=4.0, 12.9 Hz, 1H), 1.98-1.89 (m, 1H), 1.53-1.44 (m, 3H), 1.35 (br d, J=7.4 Hz, 2H), 1.18-1.09 (m, 4H), 0.88 (s, 3H), 0.86 (s, 3H). MS (ESI) m/z 464.3 [M+H]⁺

### Example 54. Synthesis of Compound 54 and 55

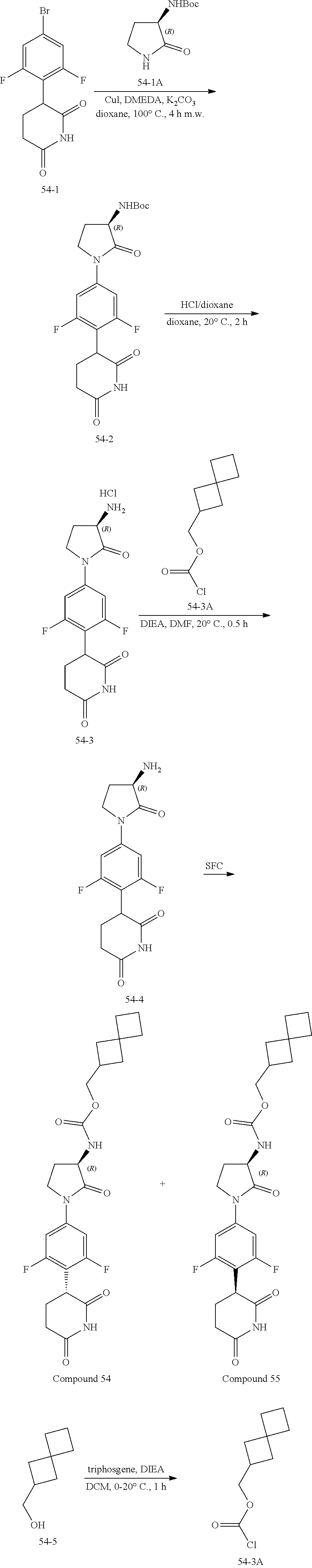

### Step 1. Procedure for Preparation of Compound 2—tert-butyl ((3R)-1-(4-(2,6-dioxopiperidin-3-yl)-3,5-difluorophenyl)-2-oxopyrrolidin-3-yl)carbamate

To a solution of 3-(4-bromo-2,6-difluorophenyl)piperidine-2,6-dione (200 mg, 658 umol, 1.00 eq) in dioxane (3.00 mL) were added CuI (25.1 mg, 132 umol, 0.200 eq), K2CO3 (273 mg, 1.97 mmol, 3.00 eq), (R)-tert-butyl (2-oxopyrrolidin-3-yl)carbamate (132 mg, 658 umol, 1.00 eq) and DMEDA (29.0 mg, 329 umol, 35.4 uL, 0.500 eq). The reaction mixture was stirred at 130° C. for 4 h under nitrogen atmosphere by microwave. The reaction mixture was filtered and the filtrate was concentrated under reduced pressure to give a residue. The residue was purified by reverse phase chromatography (C18, 40 g; condition: water/acetonitrile=100:0 to 50:50, 0.1% formic acid) and lyophilized to afford tert-butyl ((3R)-1-(4-(2,6-dioxopiperidin-3-yl)-3,5-difluorophenyl)-2-oxopyrrolidin-3-yl)carbamate (120 mg, 283 umol, 43% yield) as an off-white solid.

¹H NMR (400 MHz, DMSO-d₆) δ=10.96 (s, 1H), 7.52 (s, 1H), 7.49 (s, 1H), 7.30 (br d, J=8.5 Hz, 1H), 4.43-4.31 (m, 1H), 4.21 (dd, J=5.1, 12.6 Hz, 1H), 3.84-3.74 (m, 1H), 3.73-3.64 (m, 1H), 2.86-2.75 (m, 1H), 2.55 (br d, J=3.1 Hz, 1H), 2.40-2.34 (m, 1H), 2.19-2.09 (m, 1H), 2.01 (br d, J=5.8 Hz, 1H), 1.98-1.88 (m, 1H), 1.40 (s, 9H).

### Step 2. Procedure for Preparation of Compound 3—3-(4-((R)-3-amino-2-oxopyrrolidin-1-yl)-2,6-difluorophenyl)piperidine-2,6-dione

To a solution of tert-butyl ((3R)-1-(4-(2,6-dioxopiperidin-3-yl)-3,5-difluorophenyl)-2-oxopyrrolidin-3-yl)carbamate (120 mg, 283 umol, 1.00 eq) in dioxane (2.00 mL) was added hydrochloric acid/dioxane (4.00 M, 2.00 mL, 28.2 eq). The reaction mixture was stirred at 20° C. for 2 h. The reaction mixture was concentrated under reduced pressure to afford 3-(4-((R)-3-amino-2-oxopyrrolidin-1-yl)-2,6-difluorophenyl)piperidine-2,6-dione (110 mg, crude, hydrochloride) as a yellow solid.

¹H NMR (400 MHz, DMSO-d₆) δ=10.98 (s, 1H), 8.62 (br s, 2H), 7.55 (s, 1H), 7.52 (s, 1H), 4.33-4.20 (m, 2H), 3.96-3.90 (m, 1H), 3.86-3.77 (m, 1H), 2.88-2.77 (m, 1H), 2.56 (br d, J=3.4 Hz, 1H), 2.43-2.40 (m, 1H), 2.21-2.12 (m, 1H), 2.10 (br d, J=10.9 Hz, 1H), 2.05-1.99 (m, 1H).

### Step 3. Procedure for Preparation of Compound 3A—spiro[3.3]heptan-2-ylmethyl carbonochloridate

To a solution of spiro[3.3]heptan-2-ylmethanol (45.0 mg, 357 umol, 1.00 eq) in dichloromethane (1.00 mL) were added bis(trichloromethyl) carbonate (106 mg, 357 umol, 1.00 eq) and N,N-diisopropylethylamine (92.2 mg, 713 umol, 124 uL, 2.00 eq) at 0° C. The reaction mixture was stirred at 20° C. for 1 h. The reaction mixture was concentrated under reduced pressure to afford spiro[3.3]heptan-2-ylmethyl carbonochloridate (67.0 mg, crude) as yellow oil.

### Step 4. Procedure for Preparation of spiro[3.3]heptan-2-ylmethyl ((R)-1-(4-((R)-2,6-dioxopiperidin-3-yl)-3,5-difluorophenyl)-2-oxopyrrolidin-3-yl)carbamate and spiro[3.3]heptan-2-ylmethyl ((R)-1-(4-((S)-2,6-dioxopiperidin-3-yl)-3,5-difluorophenyl)-2-oxopyrrolidin-3-yl)carbamate

To a solution of 3-(4-((R)-3-amino-2-oxopyrrolidin-1-yl)-2,6-difluorophenyl)piperidine-2,6-dione (60.0 mg, 167 umol, 1.00 eq, hydrochloride) in dimethylformamide (0.500 mL) were added N,N-diisopropylethylamine (64.7 mg, 500 umol, 87.2 uL, 3.00 eq) and spiro[3.3]heptan-2-ylmethyl carbonochloridate (62.9 mg, 334 umol, 2.00 eq). The reaction mixture was stirred at 20° C. for 0.5 h. The reaction mixture was filtered. The filtrate was purified by reverse phase chromatography (C18, 40 g; condition: water/acetonitrile=100:0 to 40:60, 0.1% formic acid) and lyophilized to give a crude product. The crude product was purified Prep-HPLC (column: DAICEL CHIRALPAK AD (250 mm*30 mm, 10 um); mobile phase: [isopropanol-acetonitrile]; B %: 60%-60%, A6; 44 min) and concentrated under reduced pressure to give two parts. The first part was purified by Prep-HPLC (column: Welch Xtimate C18 150*25 mm*5 um; mobile phase: [water (formic acid)-acetonitrile]; B %: 43%-73%, 9 min) and lyophilized to afford spiro[3.3]heptan-2-ylmethyl ((R)-1-(4-((R)-2,6-dioxopiperidin-3-yl)-3,5-difluorophenyl)-2-oxopyrrolidin-3-yl)carbamate (8.25 mg, 13.88 umol, 8% yield, 80% purity) as a white solid. The another part was purified by Prep-HPLC (column: Welch Xtimate C18 150*25 mm*5 um; mobile phase: [water (formic acid)-acetonitrile]; B %: 43%-73%, 9 min) and lyophilized to afford spiro[3.3]heptan-2-ylmethyl ((R)-1-(4-((S)-2,6-dioxopiperidin-3-yl)-3,5-difluorophenyl)-2-oxopyrrolidin-3-yl)carbamate (7.67 mg, 15.65 umol, 9% yield, 97% purity) as a white solid.

¹H NMR (400 MHz, DMSO-d₆) δ=10.94 (s, 1H), 7.58 (br d, J=8.5 Hz, 1H), 7.51 (s, 1H), 7.48 (s, 1H), 4.46-4.33 (m, 1H), 4.26-4.17 (m, 1H), 3.96-3.85 (m, 2H), 3.83-3.75 (m, 1H), 3.74-3.65 (m, 1H), 2.87-2.76 (m, 1H), 2.57-2.52 (m, 1H), 2.39-2.34 (m, 2H), 2.18-2.12 (m, 1H), 2.08-1.93 (m, 6H), 1.92-1.86 (m, 2H), 1.79-1.69 (m, 4H). SFC: RT:1.258 min. MS (ESI) m/z 475.9 [M+H]⁺

¹H NMR (400 MHz, DMSO-d₆) δ=10.94 (s, 1H), 7.58 (br d, J=8.9 Hz, 1H), 7.51 (s, 1H), 7.48 (s, 1H), 4.40 (q, J=9.3 Hz, 1H), 4.27-4.16 (m, 1H), 3.96-3.86 (m, 2H), 3.83-3.75 (m, 1H), 3.74-3.64 (m, 1H), 2.87-2.75 (m, 1H), 2.58-2.52 (m, 1H), 2.41-2.34 (m, 2H), 2.20-2.09 (m, 1H), 2.08-1.92 (m, 6H), 1.91-1.85 (m, 2H), 1.81-1.67 (m, 4H). SFC: RT:2.629 min. MS (ESI) m/z 476.0 [M+H]⁺.

### Example 55. Synthesis of Compound 56

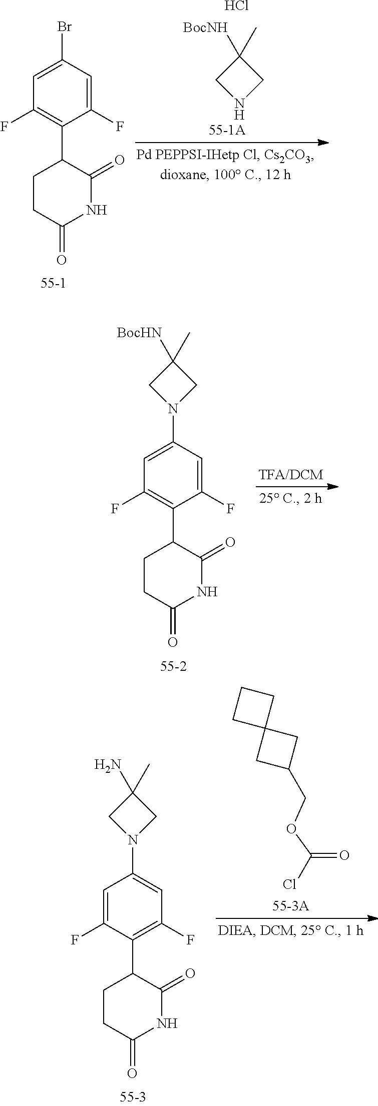

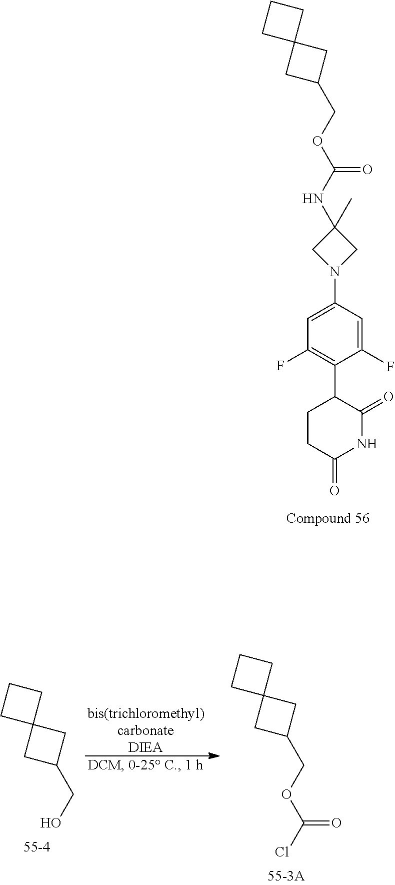 [SMILES: CC1(NC(=O)OCC2CC3(CCC3)C2)CN(c2cc(F)c(C3CCC(=O)NC3=O)c(F)c2)C1 ; OCC1CC2(CCC2)C1 ; O=C(Cl)OCC1CC2(CCC2)C1]

### Step 1. Procedure for Preparation of Compound 3A—spiro[3.3]heptan-2-ylmethyl carbonochloridate

To a solution of spiro[3.3]heptan-2-ylmethanol (30.0 mg, 238 umol, 1.00 eq) in dichloromethane (2.00 mL) were added bis(trichloromethyl) carbonate (113 mg, 380 umol, 1.60 eq) and N,N-diisopropylethylamine (61.5 mg, 475 umol, 82.8 uL, 2.00 eq) at 0° C. The mixture was stirred at 25° C. for 1 h. The reaction mixture was filtered and concentrated under reduced pressure to afford spiro[3.3]heptan-2-ylmethyl carbonochloridate (40.0 mg, crude) as a yellow solid.

### Step 2. Procedure for Preparation of Compound 2—tert-butyl (1-(4-(2,6-dioxopiperidin-3-yl)-3,5-difluorophenyl)-3-methylazetidin-3-yl)carbamate

A mixture of 3-(4-bromo-2,6-difluorophenyl)piperidine-2,6-dione (200 mg, 658 umol, 1.00 eq), tert-butyl (3-methylazetidin-3-yl)carbamate (220 mg, 987 umol, 1.50 eq, hydrochloride), cesium carbonate (643 mg, 1.97 mmol, 3.00 eq) and [1,3-bis[2,6-bis(1-propylbutyl)phenyl]-4,5-dichloro-imidazol-2-ylidene]-dichloro-(3-chloropyridin-1-ium-1-yl)palladium (64.0 mg, 65.8 umol, 0.100 eq) in dioxane (3.00 mL) was degassed and purged with nitrogen atmosphere for 3 times. The mixture was stirred at 100° C. for 12 h under nitrogen atmosphere. The reaction mixture was filtered and concentrated under reduced pressure to afford a residue. The residue was purified by reverse phase chromatography (C18, 40 g; condition: water/acetonitrile=100:0 to 0:100, 0.1% formic acid) and lyophilized to afford tert-butyl (1-(4-(2,6-dioxopiperidin-3-yl)-3,5-difluorophenyl)-3-methylazetidin-3-yl)carbamate (170 mg, 415 umol, 63% yield) as a white solid.

¹H NMR (400 MHz, DMSO-d₆) δ=10.85 (s, 1H), 7.40 (br s, 1H), 6.14 (d, J=11.3 Hz, 2H), 4.03 (br dd, J=4.9, 12.3 Hz, 1H), 3.85 (br d, J=6.5 Hz, 2H), 3.68 (d, J=7.8 Hz, 2H), 2.84-2.72 (m, 1H), 2.08 (s, 2H), 1.98-1.89 (m, 1H), 1.49 (s, 3H), 1.39 (s, 9H)

### Step 3. Procedure for Preparation of Compound 3—3-(4-(3-amino-3-methylazetidin-1-yl)-2,6-difluorophenyl)piperidine-2,6-dione

To a solution of tert-butyl (1-(4-(2,6-dioxopiperidin-3-yl)-3,5-difluorophenyl)-3-methylazetidin-3-yl)carbamate (120 mg, 293 umol, 1.00 eq) in dichloromethane (2.50 mL) was added trifluoroacetic acid (1.85 g, 16.2 mmol, 1.20 mL, 55.3 eq) at 25° C. Then the mixture was stirred at 25° C. for 2 h. The reaction mixture was concentrated under reduced pressure to afford 3-(4-(3-amino-3-methylazetidin-1-yl)-2,6-difluorophenyl)piperidine-2,6-dione (80.0 mg, 259 umol, 88% yield) as a white solid.

¹H NMR (400 MHz, DMSO-d₆) δ=10.88 (s, 1H), 8.37 (br s, 2H), 6.30 (d, J=10.9 Hz, 2H), 4.06 (br dd, J=4.9, 12.3 Hz, 1H), 3.95-3.88 (m, 2H), 3.88-3.80 (m, 2H), 3.11 (br d, J=6.3 Hz, 1H), 2.81-2.75 (m, 1H), 2.11-2.03 (m, 1H), 2.00-1.93 (m, 1H), 1.56 (s, 3H).

### Step 3. Procedure for Preparation of spiro[3.3]heptan-2-ylmethyl (1-(4-(2,6-dioxopiperidin-3-yl)-3,5-difluorophenyl)-3-methylazetidin-3-yl)carbamate

To a solution of 3-(4-(3-amino-3-methylazetidin-1-yl)-2,6-difluorophenyl)piperidine-2,6-dione (30.0 mg, 97.0 umol, 1.00 eq) in dichloromethane (1.00 mL) were added N,N-diisopropylethylamine (12.5 mg, 97.0 umol, 16.9 uL, 1.00 eq) and spiro[3.3]heptan-2-ylmethyl carbonochloridate (27.45 mg, 145.48 umol, 1.5 eq) at 25° C. Then the mixture was stirred at 25° C. for 1 h. The reaction mixture was filtered and concentrated under reduced pressure to give a residue. The residue was purified by reverse phase chromatography (C18, 40 g; condition: water/acetonitrile=100:0 to 0:100, 0.1% formic acid) and lyophilized to afford spiro[3.3]heptan-2-ylmethyl (1-(4-(2,6-dioxopiperidin-3-yl)-3,5-difluorophenyl)-3-methylazetidin-3-yl)carbamate (13.75 mg, 29.8 umol, 31% yield, 96% purity) as a white solid.

¹H NMR (400 MHz, DMSO-d₆) δ=10.86 (s, 1H), 7.72 (br s, 1H), 6.15 (s, 1H), 6.13 (s, 1H), 4.03 (br dd, J=5.0, 12.5 Hz, 1H), 3.93-3.80 (m, 4H), 3.70 (d, J=7.6 Hz, 2H), 2.81-2.71 (m, 1H), 2.37-2.31 (m, 1H), 2.14-1.90 (m, 7H), 1.89-1.82 (m, 2H), 1.74 (br dd, J=7.3, 15.0 Hz, 4H), 1.49 (s, 3H). MS (ESI) m/z 462.2 [M+H]⁺

### Example 56. Synthesis of Compound 57

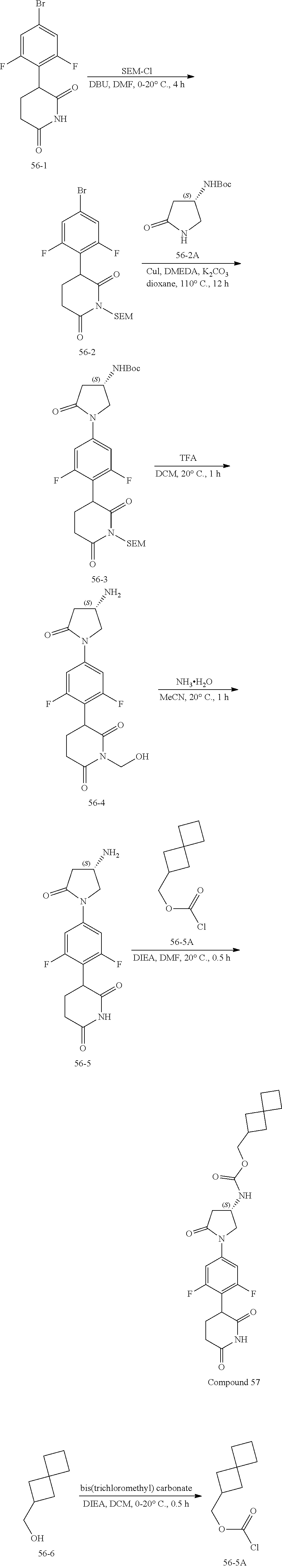

### Step 1. Procedure for Preparation of Compound 2—3-(4-bromo-2,6-difluorophenyl)-1-((2-(trimethylsilyl)ethoxy)methyl)piperidine-2,6-dione

To a mixture of 3-(4-bromo-2,6-difluorophenyl)piperidine-2,6-dione (2.00 g, 6.58 mmol, 1.00 eq) in dimethylformamide (20.0 mL) was added 1,8-diazabicyclo[5.4.0]undec-7-ene (2.00 g, 13.2 mmol, 1.98 mL, 2.00 eq), then (2-(chloromethoxy)ethyl)trimethylsilane (1.97 g, 11.8 mmol, 2.10 mL, 1.80 eq) was dropwised at 0° C. The reaction mixture was stirred at 20° C. for 4 h. The mixture was added water (20 mL) and extracted with ethyl acetate (3×30 mL). The combined organic layers were washed with brine (2×30 mL), dried over anhydrous sodium sulfate, filtered and concentrated under reduced pressure to afford 3-(4-bromo-2,6-difluorophenyl)-1-((2-(trimethylsilyl)ethoxy)methyl)piperidine-2,6-dione (2.80 g, 6.45 mmol, 98% yield) as brown oil.

¹H NMR (400 MHz, DMSO-d₆) δ=7.52 (br d, J=8.5 Hz, 2H), 5.10 (s, 2H), 4.41 (br dd, J=4.4, 12.8 Hz, 1H), 3.55-3.49 (m, 2H), 3.07-2.86 (m, 2H), 2.36-2.28 (m, 1H), 2.05-2.01 (m, 1H), 0.83 (br t, J=8.0 Hz, 2H), −0.03 (s, 9H).

### Step 2. Procedure for Preparation of Compound 3—tert-butyl ((3S)-1-(4-(2,6-dioxo-1-((2-(trimethylsilyl)ethoxy)methyl)piperidin-3-yl)-3,5-difluorophenyl)-5-oxopyrrolidin-3-yl)carbamate

To a solution of 3-(4-bromo-2,6-difluoro-phenyl)-1-(2-trimethylsilylethoxymethyl)piperidine-2,6-dione (195 mg, 449 umol, 1.00 eq) in dioxane (5.00 mL) were added (S)-tert-butyl (5-oxopyrrolidin-3-yl)carbamate (90.0 mg, 449 umol, 1.00 eq), copper iodide (85.6 mg, 449 umol, 1.00 eq), potassium carbonate (186 mg, 1.35 mmol, 3.00 eq) and N,N-dimethylethylenediamine (39.6 mg, 449 umol, 48.4 uL, 1.00 eq). The reaction was stirred at 110° C. for 12 h. The reaction mixture was filtered. The filtrate was purified by column chromatography (SiO₂, petroleum ether/ethyl acetate=20/1 to 3/1) and concentrated under reduced pressure to afford tert-butyl ((3S)-1-(4-(2,6-dioxo-1-((2-(trimethylsilyl)ethoxy)methyl)piperidin-3-yl)-3,5-difluorophenyl)-5-oxopyrrolidin-3-yl)carbamate (130 mg, 235 umol, 52% yield) as a white solid.

¹H NMR (400 MHz, DMSO-d₆) δ=7.48 (s, 1H), 7.45 (s, 1H), 5.10 (s, 2H), 4.36 (br dd, J=5.2, 13.2 Hz, 1H), 4.24-4.17 (m, 1H), 4.07 (br dd, J=7.0, 10.1 Hz, 1H), 3.63-3.58 (m, 2H), 3.54-3.49 (m, 2H), 2.99 (br dd, J=4.9, 9.7 Hz, 1H), 2.88 (br d, J=10.1 Hz, 1H), 2.77-2.72 (m, 1H), 2.45 (br d, J=12.8 Hz, 1H), 2.19-2.14 (m, 1H), 2.02 (br d, J=5.9 Hz, 1H), 1.39 (s, 9H), 0.86-0.81 (m, 2H), −0.02 (s, 9H).

### Step 3. Procedure for Preparation of Compound 2—3-(4-((S)-4-amino-2-oxopyrrolidin-1-yl)-2,6-difluorophenyl)-1-(hydroxymethyl)piperidine-2,6-dione

A solution of tert-butyl ((3S)-1-(4-(2,6-dioxo-1-((2-(trimethylsilyl)ethoxy)methyl)piperidin-3-yl)-3,5-difluorophenyl)-5-oxopyrrolidin-3-yl)carbamate (130 mg, 235 umol, 1.00 eq) in dichloromethane (3.00 mL) was added trifluoroacetic acid (924 mg, 8.10 mmol, 0.600 mL, 34.5 eq). The mixture was stirred at 20° C. for 1 h. The reaction mixture was concentrated under reduced pressure afford 3-(4-((S)-4-amino-2-oxopyrrolidin-1-yl)-2,6-difluorophenyl)-1-(hydroxymethyl)piperidine-2,6-dione (80.0 mg, 226 umol, 96% yield) as a white solid. MS (ESI) m/z. 353.8 [M+H]⁺

### Step 4. Procedure for Preparation of Compound 3—3-(4-((S)-4-amino-2-oxopyrrolidin-1-yl)-2,6-difluorophenyl)piperidine-2,6-dione

A solution of 3-(4-((S)-4-amino-2-oxopyrrolidin-1-yl)-2,6-difluorophenyl)-1-(hydroxymethyl)piperidine-2,6-dione (80.0 mg, 226 umol, 1.00 eq) in acetonitrile (3.00 mL) was added ammonium hydroxide (455 mg, 32.5 umol, 0.500 mL, 0.25% purity, 1.43^{e-1} eq). The mixture was stirred at 20° C. for 1 h. The reaction mixture was concentrated under reduced pressure to afford 3-(4-((S)-4-amino-2-oxopyrrolidin-1-yl)-2,6-difluorophenyl)piperidine-2,6-dione (70.0 mg, 217 umol, 96% yield) as a white solid. MS (ESI) m/z. 323.8 [M+H]⁺

### Step 5. Procedure for Preparation of Compound 3A—spiro[3.3]heptan-2-ylmethyl carbonochloridate

To a solution of spiro[3.3]heptan-2-ylmethanol (55.0 mg, 436 umol, 1.00 eq) in dichloromethane (0.500 mL) was added bis(trichloromethyl) carbonate (129 mg, 436 umol, 1.00 eq) at 0° C., then N,N-diisopropylethylamine (225 mg, 1.74 mmol, 304 uL, 4.00 eq) was added and the reaction mixture was stirred at 20° C. for 0.5 h. The reaction mixture was concentrated under reduced pressure to afford spiro[3.3]heptan-2-ylmethyl carbonochloridate (82.0 mg, crude) as a yellow solid.

### Step 6. Procedure for Preparation of spiro[3.3]heptan-2-ylmethyl ((3S)-1-(4-(2,6-dioxopiperidin-3-yl)-3,5-difluorophenyl)-5-oxopyrrolidin-3-yl)carbamate

To a solution of 3-(4-((S)-4-amino-2-oxopyrrolidin-1-yl)-2,6-difluorophenyl)piperidine-2,6-dione (70.0 mg, 217 umol, 1.00 eq) in dimethylformamide (1.00 mL) were added N,N-diisopropylethylamine (56.0 mg, 433 umol, 75.4 uL, 2.00 eq) and spiro[3.3]heptan-2-ylmethyl carbonochloridate (81.7 mg, 433 umol, 2.00 eq). The reaction mixture was stirred at 20° C. for 0.5 h. The reaction mixture was filtered. The filtrate was purified by reverse phase chromatography (C18, 40 g; condition: water/acetonitrile=100:0 to 0:100, 0.1% formic acid) and lyophilized to afford spiro[3.3]heptan-2-ylmethyl ((3S)-1-(4-(2,6-dioxopiperidin-3-yl)-3,5-difluorophenyl)-5-oxopyrrolidin-3-yl)carbamate (22.19 mg, 46.20 umol, 21% yield, 99% purity) as a white solid.

¹H NMR (400 MHz, DMSO-d₆) δ=10.95 (s, 1H), 7.77-7.65 (m, 1H), 7.47 (s, 1H), 7.44 (s, 1H), 4.25-4.17 (m, 2H), 4.07 (dd, J=7.1, 10.3 Hz, 1H), 3.89 (br d, J=6.8 Hz, 2H), 3.63 (br dd, J=2.9, 9.9 Hz, 1H), 2.90 (dd, J=8.2, 17.3 Hz, 1H), 2.85-2.76 (m, 1H), 2.55 (br d, J=2.5 Hz, 1H), 2.48-2.42 (m, 1H), 2.40-2.33 (m, 1H), 2.18-2.08 (m, 1H), 2.04 (br s, 1H), 2.00 (br d, J=8.6 Hz, 2H), 1.95 (br t, J=7.4 Hz, 2H), 1.89-1.84 (m, 2H), 1.77-1.68 (m, 4H). MS (ESI) m/z. 476.1 [M+H]⁺

### Example 57. Synthesis of Compound 58

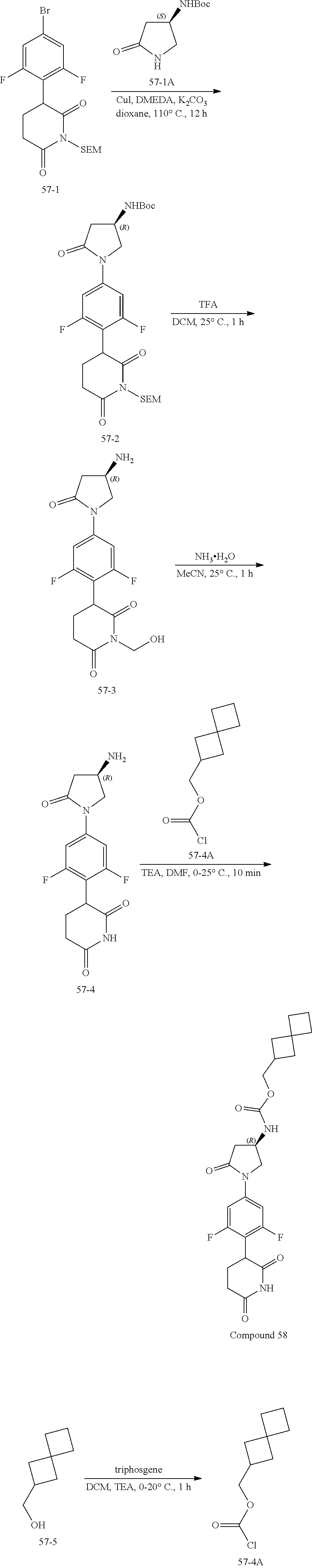

### Step 1. Procedure for Preparation of Compound 2—tert-butyl ((3R)-1-(4-(2,6-dioxo-1-((2-(trimethylsilyl)ethoxy)methyl)piperidin-3-yl)-3,5-difluorophenyl)-5-oxopyrrolidin-3-yl)carbamate

To a solution of 3-(4-bromo-2,6-difluorophenyl)-1-((2-(trimethylsilyl)ethoxy)methyl)piperidine-2,6-dione (300 mg, 691 umol, 1.00 eq), and (R)-tert-butyl (5-oxopyrrolidin-3-yl)carbamate (138 mg, 691 umol, 1.00 eq) in dioxane (20.0 mL) were added copper iodide (132 mg, 691 umol, 1.00 eq), potassium carbonate (286 mg, 2.07 mmol, 3.00 eq) and N,N′-dimethylethane-1,2-diamine (60.9 mg, 691 umol, 74.3 uL, 1.00 eq) under nitrogen atmosphere. The reaction was stirred at 110° C. for 12 h. The reaction mixture was filtered and the filtrate was concentrated under reduced pressure to give a residue. The residue was purified by column chromatography (SiO₂, petroleum ether/ethyl acetate=10/1 to 2/1) and concentrated under reduced pressure to afford tert-butyl ((3R)-1-(4-(2,6-dioxo-1-((2-(trimethylsilyl)ethoxy)methyl)piperidin-3-yl)-3,5-difluorophenyl)-5-oxopyrrolidin-3-yl)carbamate (310 mg, 560 umol, 81% yield) as yellow oil.

¹H NMR (400 MHz, DMSO-d₆) δ=7.48 (s, 1H), 7.46 (s, 1H), 5.10 (s, 2H), 4.36 (br dd, J=4.9, 12.9 Hz, 1H), 4.20 (br s, 1H), 4.10-4.06 (m, 1H), 3.60 (br dd, J=2.9, 10.1 Hz, 1H), 3.57-3.48 (m, 2H), 3.03-2.92 (m, 1H), 2.88 (dd, J=8.1, 17.4 Hz, 1H), 2.73 (br d, J=17.1 Hz, 1H), 2.45 (dd, J=4.4, 17.3 Hz, 2H), 2.24-2.09 (m, 1H), 1.39 (s, 9H), 0.83 (br d, J=8.0 Hz, 2H), 0.05-0.09 (m, 9H).

### Step 2. Procedure for Preparation of Compound 3—-(4-((R)-4-amino-2-oxopyrrolidin-1-yl)-2,6-difluorophenyl)-1-(hydroxymethyl)piperidine-2,6-dione

To a solution of tert-butyl ((3R)-1-(4-(2,6-dioxo-1-((2-(trimethylsilyl)ethoxy)methyl)piperidin-3-yl)-3,5-difluorophenyl)-5-oxopyrrolidin-3-yl)carbamate (100 mg, 181 umol, 1.00 eq) in dichloromethane (1.00 mL) was added trifluoroacetic acid (154 mg, 1.35 mmol, 100 uL, 7.48 eq). The reaction was stirred at 25° C. for 1 h. The reaction mixture was concentrated under reduced pressure to afford 3-(4-((R)-4-amino-2-oxopyrrolidin-1-yl)-2,6-difluorophenyl)-1-(hydroxymethyl)piperidine-2,6-dione (63.0 mg, 178 umol, 99% yield) as a white solid. MS (ESI) m/z. 375.9 [M+Na]⁺

### Step 3. Procedure for Preparation of Compound 4—3-(4-((R)-4-amino-2-oxopyrrolidin-1-yl)-2,6-difluorophenyl)piperidine-2,6-dione

To a solution of 3-(4-((R)-4-amino-2-oxopyrrolidin-1-yl)-2,6-difluorophenyl)-1-(hydroxymethyl)piperidine-2,6-dione (63.0 mg, 178 umol, 1.00 eq) in acetonitrile (1.00 mL) was added ammonium hydroxide (182 mg, 1.30 mmol, 200 uL, 25% purity, 7.28 eq). The reaction was stirred at 25° C. for 1 h. The reaction mixture was concentrated under reduced pressure to afford 3-(4-((R)-4-amino-2-oxopyrrolidin-1-yl)-2,6-difluorophenyl)piperidine-2,6-dione (57.0 mg, 176 umol, 99% yield) as a white solid. MS (ESI) m/z. 324.0 [M+H]⁺

### Step 4. Procedure for Preparation of Compound 4A—spiro[3.3]heptan-2-ylmethyl carbonochloridate

To a solution of spiro[3.3]heptan-2-ylmethanol (40.0 mg, 317 umol, 1.00 eq) in dichloromethane (1.00 mL) were added bis(trichloromethyl) carbonate (94.1 mg, 317 umol, 1.00 eq) and triethylamine (64.2 mg, 634 umol, 88.2 uL, 2.00 eq) at 0° C. The reaction mixture was stirred at 20° C. for 1 h. The reaction mixture was concentrated under reduced pressure to afford spiro[3.3]heptan-2-ylmethyl carbonochloridate (58.0 mg, crude) as a white solid.

### Step 5. Procedure for Preparation of spiro[3.3]heptan-2-ylmethyl ((3R)-1-(4-(2,6-dioxopiperidin-3-yl)-3,5-difluorophenyl)-5-oxopyrrolidin-3-yl)carbamate

To a solution of spiro[3.3]heptan-2-ylmethyl carbonochloridate (55.0 mg, 153 umol, 1.00 eq, hydrochloride) in dimethylformamide (1.00 mL) were added spiro[3.3]heptan-2-ylmethyl carbonochloridate (58.0 mg, 306 umol, 2.00 eq) and triethylamine (46.4 mg, 459 umol, 63.8 uL, 3.00 eq). The reaction mixture was stirred at 25° C. for 10 min. The reaction mixture was filtered. The filtrate was purified by Prep-HPLC (column: Phenomenex luna C18 150*25 mm*10 um; mobile phase: [water (formic acid)-acetonitrile]; B %: 31%-61%, 10 min) and lyophilized to afford spiro[3.3]heptan-2-ylmethyl ((3R)-1-(4-(2,6-dioxopiperidin-3-yl)-3,5-difluorophenyl)-5-oxopyrrolidin-3-yl)carbamate (10.41 mg, 21.67 umol, 14% yield, 99% purity) as a white solid.

¹H NMR (400 MHz, DMSO-d₆) δ=10.95 (s, 1H), 7.71 (br d, J=5.9 Hz, 1H), 7.47 (s, 1H), 7.44 (s, 1H), 4.30-4.16 (m, 2H), 4.08 (dd, J=6.9, 10.3 Hz, 1H), 3.89 (br d, J=6.9 Hz, 2H), 3.63 (dd, J=3.1, 10.3 Hz, 1H), 2.90 (dd, J=8.2, 17.3 Hz, 1H), 2.85-2.75 (m, 1H), 2.55 (br d, J=3.3 Hz, 1H), 2.48-2.42 (m, 1H), 2.35 (td, J=7.5, 14.7 Hz, 1H), 2.19-2.06 (m, 1H), 2.06-1.92 (m, 5H), 1.91-1.83 (m, 2H), 1.80-1.64 (m, 4H). MS (ESI) m/z. 476.1 [M+H]⁺

### Example 58. Synthesis of Compound 59

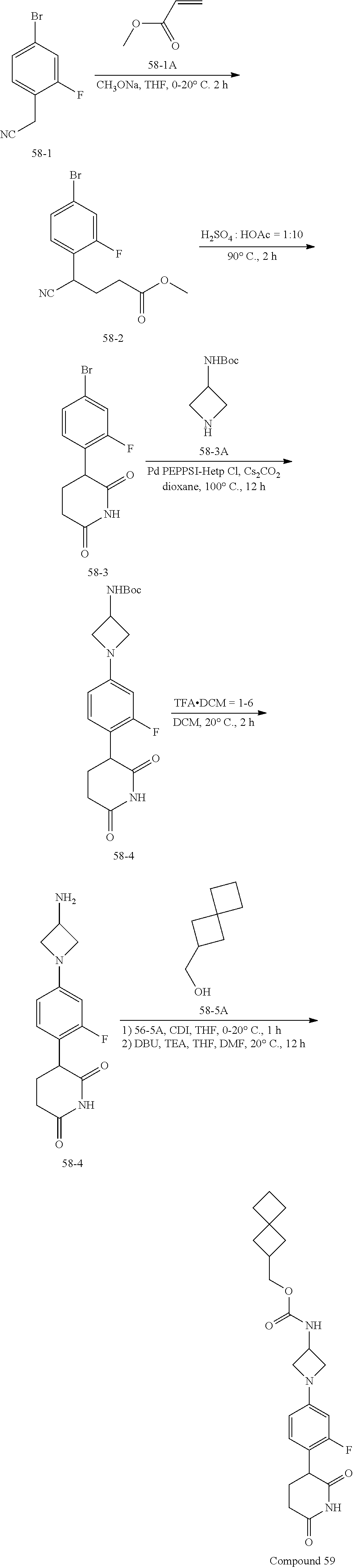

### Step 1. Procedure for Compound 2—methyl 4-(4-bromo-2-fluorophenyl)-4-cyanobutanoate

To a solution of 2-(4-bromo-2-fluorophenyl)acetonitrile (10.0 g, 46.7 mmol, 1.00 eq) in tetrahydrofuran (100 mL) was added methyl acrylate (4.42 g, 51.3 mmol, 4.63 mL, 1.10 eq) at 0° C. The mixture was added sodium methoxide (504 mg, 9.34 mmol, 0.200 eq). The mixture was stirred at 20° C. for 2 h. The mixture was quenched with saturated ammonium chloride aqueous solution (100 mL) and extracted with ethyl acetate (3×50 mL). The combined organic layers were dried over anhydrous sodium sulfate, filtered and concentrated under reduced pressure. The residue was purified by column chromatography (SiO₂, petroleum ether/ethyl acetate=20/0 to 10/1) to afford methyl 4-(4-bromo-2-fluorophenyl)-4-cyanobutanoate (8.40 g, 27.9 mmol, 59% yield) as a white solid.

¹H NMR (400 MHz, DMSO-d₆) δ=7.69-7.62 (m, 1H), 7.53-7.49 (m, 1H), 7.48-7.43 (m, 1H), 4.46 (t, J=7.5 Hz, 1H), 3.58 (d, J=2.1 Hz, 3H), 2.47-2.41 (m, 2H), 2.25-2.06 (m, 2H).

### Step 2. Procedure for Preparation of Compound 3—3-(4-bromo-2-fluorophenyl)piperidine-2,6-dione

To a solution of methyl 4-(4-bromo-2-fluorophenyl)-4-cyanobutanoate (7.40 g, 24.6 mmol, 1.00 eq) in acetic acid (70.0 mL) was added sulfuric acid (12.8 g, 131 mmol, 7.00 mL, 5.33 eq). The mixture was stirred at 90° C. for 2 h. The reaction mixture was poured into ice water (200 mL) and filtered. The filter cake was concentrated under reduced pressure to give 3-(4-bromo-2-fluorophenyl)piperidine-2,6-dione (7.00 g, 24.4 mmol, 99% yield) as an white solid.

¹H NMR (400 MHz, DMSO-d₆) δ=10.91 (s, 1H), 7.56-7.51 (m, 1H), 7.43-7.38 (m, 1H), 7.34-7.27 (m, 1H), 4.11-4.03 (m, 1H), 2.79-2.69 (m, 1H), 2.57-2.52 (m, 1H), 2.26-2.14 (m, 1H), 2.03-1.95 (m, 1H).

### Step 3. Procedure for Compound 4—tert-butyl (1-(4-(2,6-dioxopiperidin-3-yl)-3-fluorophenyl)azetidin-3-yl)carbamate

To a solution of 3-(4-bromo-2-fluorophenyl)piperidine-2,6-dione (500 mg, 1.75 mmol, 1.00 eq) in dioxane (10.0 mL) were added tert-butyl azetidin-3-ylcarbamate (300 mg, 1.75 mmol, 1.00 eq), cesium carbonate (1.71 g, 5.24 mmol, 3.00 eq) and [1,3-bis[2,6-bis(1-propylbutyl)phenyl]-4,5-dichloro-imidazol-2-ylidene]-dichloro-(3-chloropyridin-1-ium-1-yl)palladium (85.0 mg, 87.3 umol, 0.0500 eq) under nitrogen atmosphere. The mixture was stirred at 100° C. for 12 h under nitrogen atmosphere. The mixture was filtered and the filtrate was concentrated under reduced pressure to give a residue. The residue was purified by column chromatography (SiO₂, petroleum ether/ethyl acetate=10/1 to 1/1) to give a crude product. The crude product was purified by Prep-HPLC (column: Phenomenex luna C18 150*25 mm*10 um; mobile phase: [water (formic acid)-acetonitrile]; B %: 33%-63%, 10 min) and lyophilized to afford tert-butyl (1-(4-(2,6-dioxopiperidin-3-yl)-3-fluorophenyl)azetidin-3-yl)carbamate (206 mg, 545 umol, 31% yield) as a brown solid.

¹H NMR (400 MHz, DMSO-d₆) δ=10.8 (s, 1H), 7.53 (br d, J=7.4 Hz, 1H), 7.04 (t, J=8.5 Hz, 1H), 6.29-6.16 (m, 2H), 4.48-4.32 (m, 1H), 3.90-3.81 (m, 1H), 3.56 (t, J=6.8 Hz, 2H), 2.79-2.53 (m, 2H), 2.19-2.06 (m, 1H), 1.98-1.90 (m, 1H), 1.39 (s, 9H).

### Step 4. Procedure for Compound 5—3-(4-(3-aminoazetidin-1-yl)-2-fluorophenyl)piperidine-2,6-dione

To a solution of tert-butyl (1-(4-(2,6-dioxopiperidin-3-yl)-3-fluorophenyl)azetidin-3-yl)carbamate (86.0 mg, 227 umol, 1.00 eq) in dichloromethane (1.00 mL) was added trifluoroacetic acid (308 mg, 2.70 mmol, 0.200 mL, 42.4 eq). The mixture was stirred at 20° C. for 2 h. The mixture was concentrated under reduced pressure to give 3-(4-(3-aminoazetidin-1-yl)-2-fluorophenyl)piperidine-2,6-dione (89.0 mg, crude, trifluoroacetate) as colorless oil. MS (ESI) m/z 277.9 [M+H]⁺

### Step 5. Procedure for spiro[3.3]heptan-2-ylmethyl (1-(4-(2,6-dioxopiperidin-3-yl)-3-fluorophenyl)azetidin-3-yl)carbamate

To a solution of spiro[3.3]heptan-2-ylmethanol (50.0 mg, 396 umol, 1.00 eq) in tetrahydrofuran (0.500 mL) was added di(1H-imidazol-1-yl)methanone (64.2 mg, 396 umol, 1.00 eq) at 0° C. The mixture was stirred at 20° C. for 1 h. The resulting mixture was added to a solution of 3-(4-(3-aminoazetidin-1-yl)-2-fluorophenyl)piperidine-2,6-dione (88.8 mg, 227 umol, 1.00 eq, trifluoroacetate), 2,3,4,6,7,8,9,10-octahydropyrimido[1,2-a]azepine (34.5 mg, 227 umol, 34.2 uL, 1.00 eq) and triethylamine (22.9 mg, 227 umol, 31.6 uL, 1.00 eq) in tetrahydrofuran (0.250 mL) and dimethylformamide (0.250 mL). The mixture was stirred at 20° C. for 12 h. The mixture was filtered and the filtrate was concentrated under reduced pressure to give a residue. The residue was purified by Prep-HPLC (column: Phenomenex luna C18 150*25 mm*10 um; mobile phase: [water (formic acid)-acetonitrile]; B %: 46%-76%, 10 min) and lyophilized to afford spiro[3.3]heptan-2-ylmethyl (1-(4-(2,6-dioxopiperidin-3-yl)-3-fluorophenyl)azetidin-3-yl)carbamate (12.21 mg, 28.15 umol, 12% yield, 99% purity) as an off-white solid.

¹H NMR (400 MHz, DMSO-d₆) δ=10.78 (s, 1H), 7.80 (br d, J=7.4 Hz, 1H), 7.04 (t, J=8.6 Hz, 1H), 6.27-6.19 (m, 2H), 4.46-4.35 (m, 1H), 4.06 (t, J=7.5 Hz, 2H), 3.89 (d, J=7.0 Hz, 2H), 3.88-3.82 (m, 1H), 3.58 (t, J=6.8 Hz, 2H), 2.76-2.54 (m, 2H), 2.39-2.31 (m, 1H), 2.18-2.06 (m, 1H), 2.05-1.92 (m, 5H), 1.91-1.84 (m, 2H), 1.79-1.66 (m, 4H). MS (ESI) m/z 430.1 [M+H]⁺

### Example 59. Synthesis of Compound 60

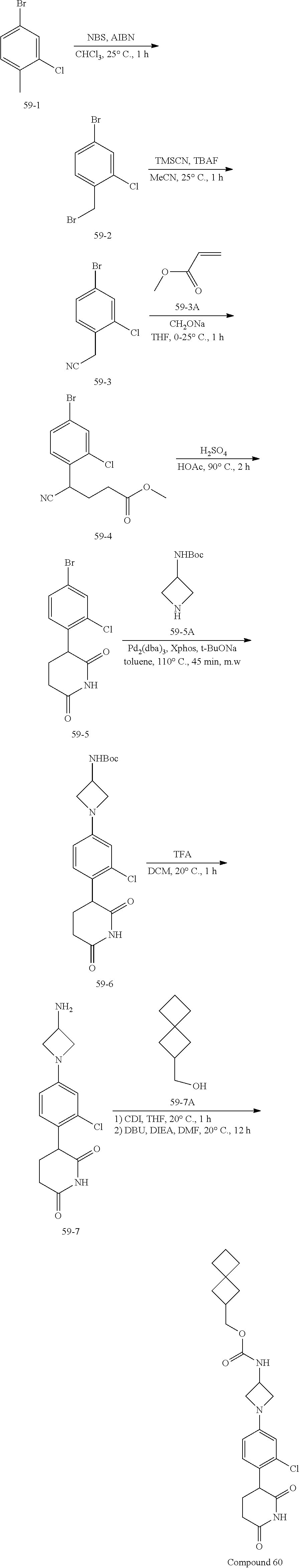

### Step 1. Procedure for Preparation of Compound 2—4-bromo-1-(bromomethyl)-2-chlorobenzene

To a solution of 4-bromo-2-chloro-1-methylbenzene (10.0 g, 48.7 mmol, 6.49 mL, 1.00 eq) in trichloromethane (100 mL) were added (E)-2,2′-(diazene-1,2-diyl)bis(2-methylpropanenitrile) (799 mg, 4.87 mmol, 0.100 eq) and N-Bromosuccinimide (8.66 g, 48.7 mmol, 1.00 eq). The mixture was stirred at 25° C. for 1 h. The reaction mixture was concentrated under reduced pressure to give a residue. The residue was purified by column chromatography (SiO₂, petroleum ether/ethyl acetate=10/1) to afford 4-bromo-1-(bromomethyl)-2-chlorobenzene (16.9 g, crude) as yellow oil.

¹H NMR (400 MHz, DMSO-d₆) δ=7.80-7.74 (m, 1H), 7.60-7.54 (m, 2H), 4.72-4.69 (m, 2H).

### Step 2. Procedure for Preparation of Compound 3—2-(4-bromo-2-chlorophenyl)acetonitrile

To a solution of 4-bromo-1-(bromomethyl)-2-chlorobenzene (16.9 g, 59.4 mmol, 1.00 eq) in acetonitrile (160 mL) were added trimethylsilyl cyanide (17.7 g, 178 mmol, 22.3 mL, 3.00 eq), tetrabutylammonium fluoride (46.6 g, 178 mmol, 42.0 mL, 3.00 eq). The mixture was stirred at 25° C. for 1 h. The reaction mixture was concentrated under reduced pressure to give a residue. The residue was purified column chromatography (SiO₂, patrolmen ether/ethyl acetate=20/1 to 10/1) to afford 2-(4-bromo-2-chlorophenyl)acetonitrile (8.78 g, 38.1 mmol, 64% yield) as a yellow oil.

¹H NMR (400 MHz, DMSO-d₆) δ=7.83-7.79 (m, 1H), 7.65-7.59 (m, 1H), 7.50 (d, J=8.3 Hz, 1H), 4.08 (s, 2H).

### Step 3. Procedure for Preparation of Compound 4—methyl 4-(4-bromo-2-chlorophenyl)-4-cyanobutanoate

To solution of 2-(4-bromo-2-chlorophenyl)acetonitrile (8.78 g, 38.1 mmol, 1.00 eq) in tetrahydrofuran (80.0 mL) were added methyl acrylate (3.61 g, 41.9 mmol, 3.77 mL, 1.10 eq) and sodium methoxide (206 mg, 3.81 mmol, 0.100 eq) at 0° C. Then the mixture was stirred at 25° C. for 1 h. The mixture was quenched with saturated ammonium chloride aqueous solution (80 mL) and extracted with ethyl acetate (3×80 mL). The combined organic layers were washed with brine (2×40 mL), dried over anhydrous sodium sulfate, filtered and concentrated under reduced pressure to afford methyl 4-(4-bromo-2-chlorophenyl)-4-cyanobutanoate (9.78 g, 30.9 mmol, 81% yield) as a yellow oil.

¹H NMR (400 MHz, DMSO-d₆) δ=7.83 (s, 1H), 7.66 (dd, J=1.9, 8.4 Hz, 1H), 7.52 (d, J=8.3 Hz, 1H), 4.54 (dd, J=6.6, 8.3 Hz, 1H), 3.58 (s, 3H), 2.49-2.43 (m, 2H), 2.24-2.12 (m, 2H).

### Step 4. Procedure for Preparation of Compound 5—3-(4-bromo-2-chlorophenyl)piperidine-2,6-dione

To a solution of methyl 4-(4-bromo-2-chlorophenyl)-4-cyanobutanoate (9.78 g, 30.9 mmol, 1.00 eq) in acetic acid (100 mL) was added sulfuric acid (18.0 g, 183 mmol, 9.78 mL, 5.94 eq). The mixture was stirred at 90° C. for 2 h. The reaction mixture was quench with (50 mL) water and then filtered. The filter cake was purified by column chromatography (SiO₂, petroleum ether/ethyl acetate=1/1 to 0/1) to afford 3-(4-bromo-2-chlorophenyl)piperidine-2,6-dione (3.86 g, 12.8 mmol, 41% yield) as a white solid.

¹H NMR (400 MHz, DMSO-d₆) δ=10.93 (s, 1H), 7.74 (d, J=2.0 Hz, 1H), 7.55 (dd, J=2.0, 8.4 Hz, 1H), 7.32 (d, J=8.4 Hz, 1H), 4.22 (dd, J=4.9, 12.6 Hz, 1H), 2.85-2.69 (m, 1H), 2.56 (br t, J=3.4 Hz, 1H), 2.34-2.22 (m, 1H), 1.99-1.93 (m, 1H).

### Step 5. Procedure for Preparation of Compound 6—tert-butyl (1-(3-chloro-4-(2,6-dioxopiperidin-3-yl)phenyl)azetidin-3-yl)carbamate

To a solution of 3-(4-bromo-2-chlorophenyl)piperidine-2,6-dione (50.0 mg, 165 umol, 1.00 eq) in toluene (1.00 mL) were added tert-butyl azetidin-3-ylcarbamate (56.9 mg, 331 umol, 2.00 eq), sodium tert-butoxide (95.3 mg, 992 umol, 6.00 eq), tris(dibenzylideneacetone)dipalladium(0) (15.1 mg, 16.5 umol, 0.100 eq) and dicyclohexyl(2′,4′,6′-triisopropyl-[1,1′-biphenyl]-2-yl)phosphine (15.8 mg, 33.1 umol, 0.200 eq). The reaction mixture was stirred at 110° C. for 45 min under nitrogen atmosphere by microwave. The reaction mixture was concentrated under reduced pressure to give a residue. The residue was dissolved in dimethylformamide (1 mL) and then filtered. The filtrate was purified by reverse phase chromatography (C18, 40 g; condition: water/acetonitrile=100:0 to 45:55, 0.1% formic acid) and lyophilized to afford tert-butyl (1-(3-chloro-4-(2,6-dioxopiperidin-3-yl)phenyl)azetidin-3-yl)carbamate (50.0 mg, 127 umol, 77% yield) as a yellow solid.

¹H NMR (400 MHz, DMSO-d₆) δ=10.81 (s, 1H), 7.53 (br d, J=7.4 Hz, 1H), 7.07 (d, J=8.4 Hz, 1H), 6.46 (d, J=2.0 Hz, 1H), 6.37 (dd, J=2.1, 8.4 Hz, 1H), 4.47-4.24 (m, 1H), 4.15-3.95 (m, 3H), 3.57 (t, J=6.8 Hz, 2H), 2.73 (ddd, J=5.3, 12.6, 17.3 Hz, 1H), 2.47 (br s, 1H), 2.21 (dq, J=4.2, 12.7 Hz, 1H), 1.97-1.87 (m, 1H), 1.39 (s, 9H).

### Step 6. Procedure for Preparation of Compound 7—3-(4-(3-aminoazetidin-1-yl)-2-chlorophenyl)piperidine-2,6-dione

To a solution of tert-butyl (1-(3-chloro-4-(2,6-dioxopiperidin-3-yl)phenyl)azetidin-3-yl)carbamate (50.0 mg, 127 umol, 1.00 eq) in dichloromethane (0.500 mL) was added trifluoroacetic acid (77.0 mg, 675 umol, 0.0500 mL, 5.32 eq). The reaction mixture was stirred at 20° C. for 1 h. The reaction mixture was concentrated under reduced pressure to afford 3-(4-(3-aminoazetidin-1-yl)-2-chlorophenyl)piperidine-2,6-dione (50.0 mg, crude, trifluoroacetate) as yellow oil. MS (ESI) m/z 293.8 [M+H]⁺

### Step 7. Procedure for Preparation of spiro[3.3]heptan-2-ylmethyl (1-(3-chloro-4-(2,6-dioxopiperidin-3-yl)phenyl)azetidin-3-yl)carbamate

To a solution of spiro[3.3]heptan-2-ylmethanol (20.0 mg, 158 umol, 1.00 eq) in tetrahydrofuran (0.300 mL) was added di(1H-imidazol-1-yl)methanone (25.7 mg, 158 umol, 1.00 eq). The reaction mixture was stirred at 20° C. for 1 h. The resulting solution was added to a mixture of 3-(4-(3-aminoazetidin-1-yl)-2-chlorophenyl)piperidine-2,6-dione (50.0 mg, 123 umol, 1.00 eq, trifluoroacetate), 2,3,4,6,7,8,9,10-octahydropyrimido [1,2-a]azepine (18.7 mg, 123 umol, 18.5 uL, 1.00 eq) and N,N-diisopropylethylamine (15.9 mg, 123 umol, 21.4 uL, 1.00 eq) in dimethylformamide (0.500 mL). The reaction mixture was stirred at 20° C. for 12 h. The reaction mixture was concentrated under reduced pressure to give a residue. The residue was dissolved in dimethylformamide (0.5 mL) and then filtered. The filtrate was purified by reverse phase chromatography (C18, 40 g; condition: water/acetonitrile=100:0 to 30:70, 0.1% formic acid) and lyophilized to afford spiro[3.3]heptan-2-ylmethyl (1-(3-chloro-4-(2,6-dioxopiperidin-3-yl)phenyl)azetidin-3-yl)carbamate (14.15 mg, 31.10 umol, 25% yield, 98% purity) as a white solid.

¹H NMR (400 MHz, DMSO-d₆) δ=10.81 (s, 1H), 7.80 (br d, J=7.8 Hz, 1H), 7.08 (d, J=8.5 Hz, 1H), 6.47 (d, J=2.4 Hz, 1H), 6.38 (dd, J=2.3, 8.4 Hz, 1H), 4.48-4.34 (m, 1H), 4.08 (t, J=7.6 Hz, 2H), 4.05-4.00 (m, 1H), 3.89 (d, J=6.9 Hz, 2H), 3.59 (t, J=6.8 Hz, 2H), 2.73 (ddd, J=5.2, 12.5, 17.4 Hz, 1H), 2.48-2.44 (m, 1H), 2.41-2.33 (m, 1H), 2.27-2.15 (m, 1H), 2.05-1.99 (m, 2H), 1.99-1.90 (m, 3H), 1.90-1.85 (m, 2H), 1.79-1.67 (m, 4H). MS (ESI) m/z 468.0 [M+Na]⁺

### Example 60. Synthesis of Compound 61

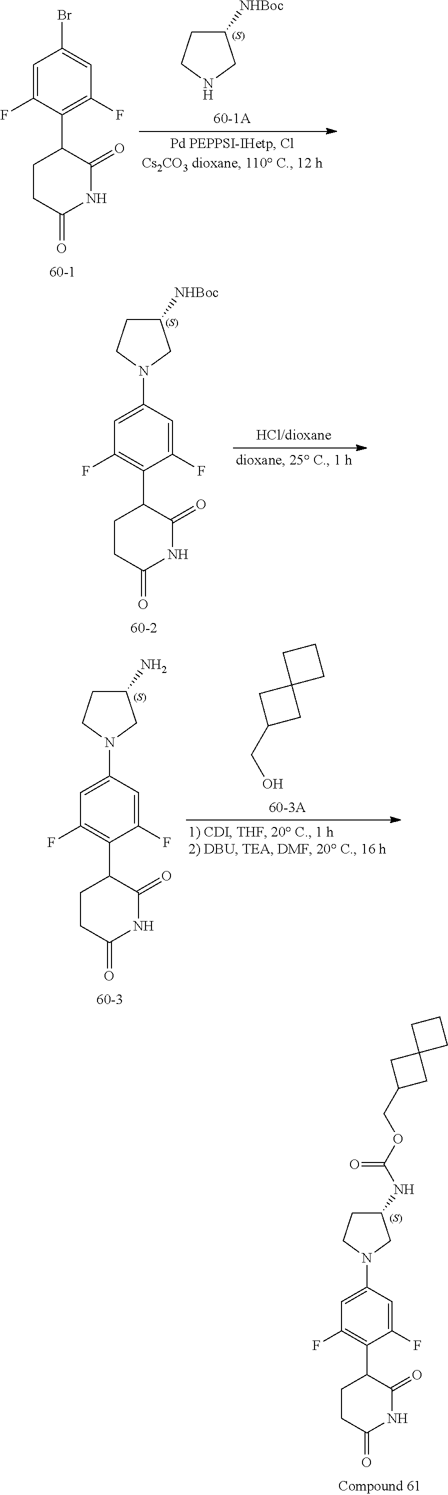

### Step 1. Procedure for Preparation of Compound 2—tert-butyl ((3S)-1-(4-(2,6-dioxopiperidin-3-yl)-3,5-difluorophenyl)pyrrolidin-3-yl)carbamate

To a solution of 3-(4-bromo-2,6-difluoro-phenyl)piperidine-2,6-dione (300 mg, 987 umol, 1.00 eq), (S)-tert-butyl pyrrolidin-3-ylcarbamate (276 mg, 1.48 mmol, 1.50 eq) in dioxane (3.00 mL) were added cesium carbonate (964 mg, 2.96 mmol, 3.00 eq) and [1,3-bis[2,6-bis(1-propylbutyl)phenyl]-4,5-dichloro-imidazol-2-ylidene]-dichloro-(3-chloropyridin-1-ium-1-yl)palladium (96.0 mg, 99.0 umol, 0.100 eq). The mixture was stirred at 110° C. for 12 h under nitrogen atmosphere. The reaction mixture was filtered. The filtrate was concentrated under reduce pressure to give a residue. The residue was purified by reverse phase chromatography (C18, 80 g; condition: water/acetonitrile=100:0 to 50:50, 0.1% formic acid) and lyophilized to afford tert-butyl ((3S)-1-(4-(2,6-dioxopiperidin-3-yl)-3,5-difluorophenyl)pyrrolidin-3-yl)carbamate (200 mg, 488 umol, 50% yield) a white solid.

¹H NMR (400 MHz, DMSO-d₆) δ=10.83 (br s, 1H), 7.19 (br d, J=5.9 Hz, 1H), 6.18 (br d, J=12.4 Hz, 2H), 4.18-3.98 (m, 2H), 3.46-3.40 (m, 1H), 3.38-3.34 (m, 1H), 3.25-3.18 (m, 1H), 3.01 (br dd, J=4.2, 9.2 Hz, 1H), 2.84-2.73 (m, 1H), 2.67 (br s, 1H), 2.15-2.03 (m, 2H), 1.95-1.83 (m, 2H), 1.39 (s, 9H).

### Step 2. Procedure for Preparation of Compound 3—3-(4-((S)-3-aminopyrrolidin-1-yl)-2,6-difluorophenyl)piperidine-2,6-dione

To a solution of tert-butyl ((3S)-1-(4-(2,6-dioxopiperidin-3-yl)-3,5-difluorophenyl)pyrrolidin-3-yl)carbamate (200 mg, 489 umol, 1.00 eq) in dioxane (2.00 mL) was added hydrochloric acid/dioxane (4.00 M) (2.00 mL). The mixture was stirred at 25° C. for 1 h. The reaction mixture was concentrated under reduce pressure to afford 3-(4-((S)-3-aminopyrrolidin-1-yl)-2,6-difluorophenyl)piperidine-2,6-dione (115 mg, 372 umol, 76% yield, hydrochloride) as a pink solid. MS (ESI) m/z 309.8 [M+H]⁺

### Step 3. Procedure for Preparation of spiro[3.3]heptan-2-ylmethyl ((3S)-1-(4-(2,6-dioxopiperidin-3-yl)-3,5-difluorophenyl)pyrrolidin-3-yl)carbamate

To a solution of spiro[3.4]octan-2-ylmethanol (50.0 mg, 396 umol, 1.00 eq) in tetrahydrofuran (0.300 mL) was added di(1H-imidazol-1-yl)methanone (96.4 mg, 594 umol, 1.50 eq). The reaction mixture was stirred at 20° C. for 1 h. The resulting solution was added to a mixture of 3-(4-((S)-3-aminopyrrolidin-1-yl)-2,6-difluorophenyl)piperidine-2,6-dione (115 mg, 372 umol, hydrochloride), 2,3,4,6,7,8,9,10-octahydropyrimido[1,2-a]azepine (55.5 mg, 365 umol, 55.0 uL, 1.00 eq) and triethylamine (37.0 mg, 3645 umol, 51.0 uL, 1.00 eq) in dimethylformamide (1.50 mL). The reaction mixture was stirred at 20° C. for 16 h. The reaction mixture was concentrated under reduced pressure to give a residue. The residue was dissolved in dimethylformamide (1.5 mL) and then filtered. The filtrate was purified by Prep-HPLC (column: Phenomenex luna C18 150*25 mm*10 um; mobile phase: [water (formic acid)-acetonitrile]; B %: 51%-81%, 10 min) and lyophilized to afford spiro[3.3]heptan-2-ylmethyl ((3S)-1-(4-(2,6-dioxopiperidin-3-yl)-3,5-difluorophenyl)pyrrolidin-3-yl) carbamate (10.00 mg, 21.67 umol, 28% yield, 99% purity) as a white solid.

¹H NMR (400 MHz, DMSO-d₆) δ=10.84 (s, 1H), 7.56-7.37 (m, 1H), 6.20 (s, 1H), 6.17 (s, 1H), 4.20-4.13 (m, 1H), 4.04-3.98 (m, 1H), 3.89 (br d, J=7.0 Hz, 2H), 3.44 (br dd, J=6.9, 9.1 Hz, 1H), 3.40-3.35 (m, 1H), 3.26-3.18 (m, 1H), 3.05 (br dd, J=4.5, 9.9 Hz, 1H), 2.85-2.72 (m, 1H), 2.57 (br s, 1H), 2.42-2.31 (m, 1H), 2.18-2.11 (m, 1H), 2.10 (br d, J=3.8 Hz, 3H), 2.00-1.90 (m, 4H), 1.87 (br d, J=7.5 Hz, 2H), 1.79-1.68 (m, 4H). MS (ESI) m/z 462.1 [M+H]⁺

### Example 61. Synthesis of Compound 62

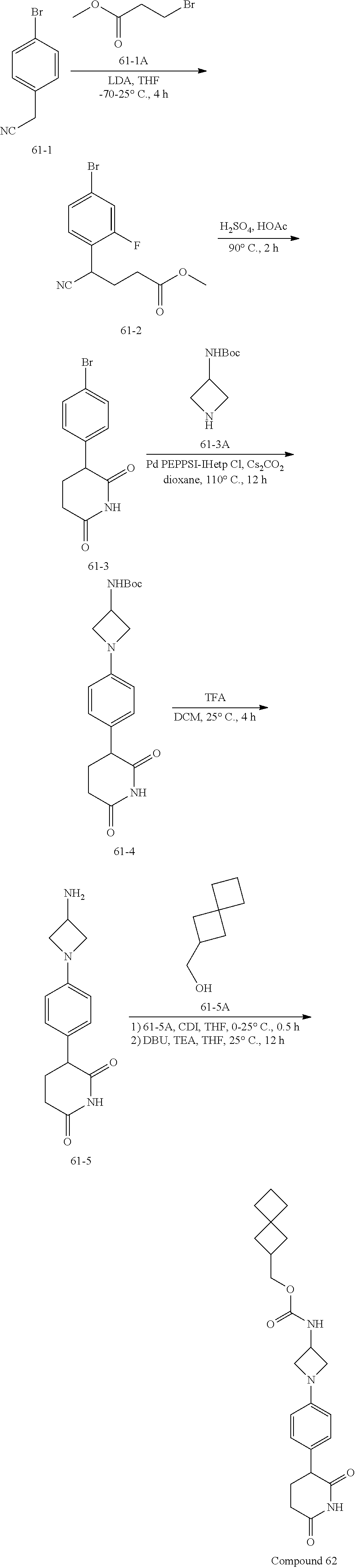

### Step 1. Procedure for Preparation of Compound 2—methyl 4-(4-bromophenyl)-4-cyanobutanoate

To the solution of 2-(4-bromophenyl)acetonitrile (5.00 g, 25.5 mmol, 1.00 eq) in tetrahydrofuran (50.0 mL) was added lithium diisopropylamide (2.00 M, 19.2 mL, 1.50 eq) at −70° C. under nitrogen atmosphere and the mixture was stirred at −70° C. for 30 min. After 30 min, methyl 3-bromopropanoate (4.69 g, 28.1 mmol, 3.06 mL, 1.10 eq) was added dropwise into the mixture at −70° C. Then the reaction was stirred at 25° C. for 3.5 h. The mixture was quenched by addition of 10 mL of saturated aqueous ammonium chloride, then extracted by ethyl acetate (2×50 mL). The combined organic layers were washed with brine (2×10 mL), and dried over anhydrous sodium sulfate, filtered and concentrate to give a residue. The residue was purified by column chromatography (SiO₂, petroleum ether/ethyl acetate=10/1) to afford methyl 4-(4-bromophenyl)-4-cyanobutanoate (2.50 g, 8.86 mmol, 35% yield) as colorless oil.

¹H NMR (400 MHz, CDCl₃) δ=7.55-7.51 (m, 2H), 7.25-7.22 (m, 2H), 3.97 (t, J=7.5 Hz, 1H), 3.69 (s, 3H), 2.56-2.43 (m, 2H), 2.19 (q, J=7.4 Hz, 2H).

### Step 2. Procedure for Preparation of Compound 3—3-(4-bromophenyl)piperidine-2,6-dione

To the solution of methyl 4-(4-bromophenyl)-4-cyanobutanoate (2.70 g, 9.57 mmol, 1.00 eq) in acetic acid (20.0 mL) was added sulfuric acid (2.00 mL). Then the reaction was stirred at 90° C. for 2 h. The reaction mixture was added dropwised into cold water and stirred for 0.5 h. The mixture was filtered. The filter cake was concentrated under reduced pressure to afford 3-(4-bromophenyl)piperidine-2,6-dione (1.60 g, 5.97 mmol, 62% yield) as a white solid.

¹H NMR (400 MHz, CDCl₃) δ=8.10 (br s, 1H), 7.52 (d, J=8.4 Hz, 2H), 7.11 (d, J=8.4 Hz, 2H), 3.74 (dd, J=5.3, 10.3 Hz, 1H), 2.80-2.65 (m, 2H), 2.32-2.21 (m, 2H).

### Step 3. Procedure for Preparation of Compound 4—tert-butyl (1-(4-(2,6-dioxopiperidin-3-yl)phenyl)azetidin-3-yl)carbamate

To the solution of 3-(4-bromophenyl)piperidine-2,6-dione (400 mg, 1.49 mmol, 1.00 eq), tert-butyl azetidin-3-ylcarbamate (308 mg, 1.79 mmol, 1.20 eq) in dioxane (20.0 mL) were added cesium carbonate (1.46 g, 4.48 mmol, 3.00 eq), 1,3-bis[2,6-bis(1-propylbutyl)phenyl]-4,5-dichloro-2H-imidazol-1-ium-2-ide;3-chloropyridine;dichloropalladium (40.0 mg, 41.1 umol, 2.76e−2 eq) under nitrogen atmosphere. Then the reaction was stirred at 110° C. for 12 h. The mixture was diluted with water (10 mL), extracted with a solution of dichloromethane/isopropanol=3/1 (2×20 mL). The combined organic layers were washed with brine (2×10 mL), dried over anhydrous sodium sulfate, filtered and concentrate to give a residue. The residue was purified by column chromatography (SiO₂, petroleum ether/ethyl acetate=1/1 to 1/2) to afford tert-butyl (1-(4-(2,6-dioxopiperidin-3-yl)phenyl)azetidin-3-yl)carbamate (140 mg, 389 umol, 26% yield) as a white solid.

¹H NMR (400 MHz, CDCl₃) δ=7.89 (br s, 1H), 7.06 (d, J=8.1 Hz, 2H), 6.46 (d, J=8.1 Hz, 2H), 5.01-4.89 (m, 1H), 4.66-4.54 (m, 1H), 4.21 (br t, J=7.3 Hz, 2H), 3.70 (dd, J=5.3, 9.4 Hz, 1H), 3.61 (br t, J=6.4 Hz, 2H), 2.76-2.61 (m, 2H), 2.27-2.18 (m, 2H), 1.46 (s, 9H).

### Step 4. Procedure for Preparation of Compound 5—3-(4-(3-aminoazetidin-1-yl)phenyl)piperidine-2,6-dione

To the solution of tert-butyl (1-(4-(2,6-dioxopiperidin-3-yl)phenyl)azetidin-3-yl)carbamate (220 mg, 612 umol, 1.00 eq) in dichloromethane (3.00 mL) was added trifluoroacetic acid (813 mg, 7.13 mmol, 528 uL, 11.7 eq). Then the reaction was stirred at 25° C. for 4 h. The reaction mixture was concentrated under reduced pressure to afford 3-(4-(3-aminoazetidin-1-yl)phenyl)piperidine-2,6-dione (158 mg, crude, trifluoroacetate) as brown oil. MS (ESI) m/z 260.1 [M+H]⁺

### Step 5. Procedure for Preparation of spiro[3.3]heptan-2-ylmethyl (1-(4-(2,6-dioxopiperidin-3-yl)phenyl)azetidin-3-yl)carbamate

To the solution of spiro[3.3]heptan-2-ylmethanol (50.0 mg, 396 umol, 1.00 eq) in tetrahydrofuran (0.500 mL) was added di(1H-imidazol-1-yl)methanone (64.2 mg, 396.2 umol, 1.00 eq) at 0° C. Then the reaction was stirred at 25° C. for 0.5 h. Then to the mixture were added tetrahydrofuran (0.500 mL), 3-(4-(3-aminoazetidin-1-yl)phenyl)piperidine-2,6-dione (108 mg, 289 umol, 1.00 eq, trifluoroacetate), triethylamine (29.3 mg, 289 umol, 40.3 uL, 1.00 eq), 2,3,4,6,7,8,9,10-octahydropyrimido[1,2-a]azepine (44.0 mg, 289 umol, 43.6 uL, 1.00 eq). Then the reaction was stirred at 25° C. for 12 h. The reaction mixture was concentrated under reduced pressure to give a residue. The residue was purified by prep-HPLC (column: Phenomenex luna C18 150*25 mm*10 um; mobile phase: [water (formic acid)-acetonitrile]; B %: 45%-75%, 10 min) and lyophilized to afford spiro[3.3]heptan-2-ylmethyl (1-(4-(2,6-dioxopiperidin-3-yl)phenyl)azetidin-3-yl)carbamate (35.16 mg, 82.03 umol, 28% yield, 96% purity) as an off-white solid.

¹H NMR (400 MHz, DMSO-d) δ=10.7 (s, 1H), 7.76 (br d, J=7.3 Hz, 1H), 7.00 (d, J=7.9 Hz, 2H), 6.39 (br d, J=8.0 Hz, 2H), 4.44-4.38 (m, 1H), 4.05 (t, J=7.3 Hz, 2H), 3.89 (br d, J=6.8 Hz, 2H), 3.70 (br dd, J=4.9, 10.6 Hz, 1H), 3.54 (br t, J=6.6 Hz, 2H), 2.65-2.57 (m, 1H), 2.47-2.42 (m, 1H), 2.37-2.32 (m, 1H), 2.13-2.08 (m, 1H), 2.04-1.95 (m, 5H), 1.89-1.86 (m, 2H), 1.76-1.70 (m, 4H). MS (ESI) m/z 412.1 [M+H]⁺

### Example 62. Synthesis of Compound 63

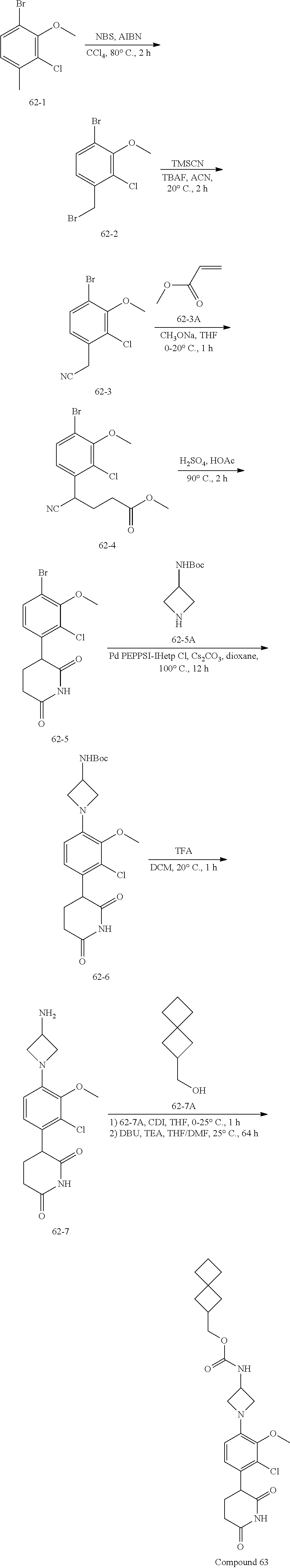

### Step 1. Procedure for Preparation of Compound 2—1-bromo-4-(bromomethyl)-3-chloro-2-methoxybenzene

To a solution of 1-bromo-3-chloro-2-methoxy-4-methyl-benzene (4.00 g, 17.0 mmol, 1.00 eq) in carbon tetrachloride (40.0 mL) were added N-bromosuccinimide (3.02 g, 17.0 mmol, 1.00 eq) and azodiisobutyronitrile (139 mg, 849 umol, 0.0500 eq). The reaction mixture was stirred at 80° C. for 2 h. The mixture was concentrated under reduced pressure to give a residue. The residue was purified by column chromatography (SiO₂, petroleum ether/ethyl acetate=1/to 10/1) to afford 1-bromo-4-(bromomethyl)-3-chloro-2-methoxybenzene (2.70 g, 8.59 mmol, 51% yield) as colorless oil.

¹H NMR (400 MHz, DMSO-d₆) δ=7.65 (d, J=8.4 Hz, 1H), 7.37 (d, J=8.4 Hz, 1H), 4.73 (s, 2H), 3.82 (s, 3H).

### Step 2. Procedure for Preparation of Compound 3—2-(4-bromo-2-chloro-3-methoxyphenyl)acetonitrile

To a solution of 1-bromo-4-(bromomethyl)-3-chloro-2-methoxybenzene (2.70 g, 8.59 mmol, 1.00 eq) in acetonitrile (5.00 mL) were added trimethylsilyl cyanide (2.56 g, 25.8 mmol, 3.22 mL, 3.00 eq) and tetrabutylammonium fluoride (1.00 M, 25.8 mL, 3.00 eq). The reaction mixture was stirred at 20° C. for 2 h. The reaction mixture was concentrated under reduced pressure to give a residue. The residue was purified by column chromatography (SiO₂, petroleum ether/ethyl acetate=1/0 to 10/1) and concentrated under reduced pressure to afford 2-(4-bromo-2-chloro-3-methoxyphenyl)acetonitrile (2.25 g, crude) as colorless oil.

¹H NMR (400 MHz, DMSO-d₆) δ=7.68 (d, J=8.4 Hz, 1H), 7.27 (d, J=8.4 Hz, 1H), 4.08 (s, 2H), 3.80 (s, 3H).

### Step 3. Procedure for Preparation of Compound 4—methyl 4-(4-bromo-2-chloro-3-methoxyphenyl)-4-cyanobutanoate

To a solution of 2-(4-bromo-2-chloro-3-methoxyphenyl)acetonitrile (2.25 g, 8.64 mmol, 1.00 eq) and methyl acrylate (818 mg, 9.50 mmol, 856 uL, 1.10 eq) in tetrahydrofuran (20.0 mL) was added sodium methoxide (46.7 mg, 864 umol, 0.100 eq) at 0° C. The reaction mixture was stirred at 20° C. for 1 h. The mixture was quenched with saturated ammonium chloride aqueous solution (30 mL) and extracted with ethyl acetate (3×30 mL). The combined organic layers were washed with brine (2×30 mL), dried over anhydrous sodium sulfate, filtered and concentrated under reduced pressure to afford methyl 4-(4-bromo-2-chloro-3-methoxyphenyl)-4-cyanobutanoate (2.90 g, 8.37 mmol, 97% yield) as colorless oil.

¹H NMR (400 MHz, DMSO-d₆) δ=7.69 (d, J=8.5 Hz, 1H), 7.27 (d, J=8.5 Hz, 1H), 4.52 (dd, J=6.6, 8.3 Hz, 1H), 3.82-3.74 (m, 3H), 3.58-3.52 (m, 3H), 2.45-2.41 (m, 2H), 2.20-2.02 (m, 2H).

### Step 4. Procedure for Preparation of Compound 5—3-(4-bromo-2-chloro-3-methoxyphenyl)piperidine-2,6-dione

To a solution of methyl 4-(4-bromo-2-chloro-3-methoxyphenyl)-4-cyanobutanoate (2.90 g, 8.37 mmol, 1.00 eq) in acetic acid (30.0 mL) was added sulfuric acid (5.48 g, 55.9 mmol, 2.98 mL, 6.68 eq). The mixture was stirred at 90° C. for 2 h. The mixture was poured into ice water (20.0 mL) and filtered. The filter cake was dried under reduced pressure to afford 3-(4-bromo-2-chloro-3-methoxyphenyl)piperidine-2,6-dione (1.49 g, 4.48 mmol, 54% yield) as a white solid.

¹H NMR (400 MHz, DMSO-d₆) δ=10.93 (s, 1H), 7.61 (d, J=8.5 Hz, 1H), 7.10 (d, J=8.4 Hz, 1H), 4.25 (dd, J=5.0, 12.4 Hz, 1H), 3.81 (s, 3H), 2.82-2.71 (m, 1H), 2.55 (br t, J=3.3 Hz, 1H), 2.30 (br dd, J=4.3, 12.9 Hz, 1H), 2.04-1.91 (m, 1H).

### Step 5. Procedure for Preparation of Compound 6—tert-butyl (1-(3-chloro-4-(2,6-dioxopiperidin-3-yl)-2-methoxyphenyl)azetidin-3-yl)carbamate

To a solution of 3-(4-bromo-2-chloro-3-methoxy-phenyl)piperidine-2,6-dione (400 mg, 1.20 mmol, 1.00 eq), tert-butyl azetidin-3-ylcarbamate (269 mg, 1.56 mmol, 1.30 eq) and cesium carbonate (1.18 g, 3.61 mmol, 3.00 eq) in dioxane (4.00 mL) were added [1,3-bis[2,6-bis(1-propylbutyl)phenyl]-4,5-dichloro-imidazol-2-ylidene]-dichloro-(3-chloropyridin-1-ium-1-yl)palladium (40.0 mg, 41.12 umol, 3.42e⁻² eq). The reaction mixture was stirred at 100° C. for 12 h under nitrogen atmosphere. The reaction mixture was filtered, and concentrated under reduced pressure to give a residue. The residue was purified by column chromatography (SiO₂, petroleum ether/ethyl acetate=100/1 to 1/1) and concentrated under reduced pressure to afford tert-butyl (1-(3-chloro-4-(2,6-dioxopiperidin-3-yl)-2-methoxyphenyl)azetidin-3-yl)carbamate (70.0 mg, 165 umol, 14% yield) as a yellow solid.

¹H NMR (400 MHz, DMSO-d₆) δ=10.82 (s, 1H), 7.50 (br d, J=7.6 Hz, 1H), 6.89 (d, J=8.4 Hz, 1H), 6.43 (d, J=8.5 Hz, 1H), 4.46-4.30 (m, 1H), 4.13 (br t, J=7.6 Hz, 2H), 4.08-4.05 (m, 1H), 3.66-3.57 (m, 5H), 2.77-2.69 (m, 1H), 2.41-2.38 (m, 1H), 2.27-2.17 (m, 1H), 1.95-1.89 (m, 1H), 1.39 (s, 9H).

### Step 6. Procedure for Preparation of Compound 7—3-(4-(3-aminoazetidin-1-yl)-2-chloro-3-methoxyphenyl)piperidine-2,6-dione

To a solution of tert-butyl (1-(3-chloro-4-(2,6-dioxopiperidin-3-yl)-2-methoxyphenyl)azetidin-3-yl)carbamate (60.0 mg, 142 umol, 1.00 eq) in dichloromethane (1.00 mL) were added trifluoroacetic acid (154 mg, 1.35 mmol, 0.100 mL, 9.54 eq). The reaction mixture was stirred at 20° C. for 1 h. The reaction mixture was concentrated under reduce pressure to afford 3-[4-(3-aminoazetidin-1-yl)-2-chloro-3-methoxy-phenyl]piperidine-2,6-dione (45.0 mg, crude) as a yellow solid.

¹H NMR (400 MHz, DMSO-d₆) δ=10.84 (s, 1H), 8.27 (br d, J=4.0 Hz, 2H), 6.94 (d, J=8.4 Hz, 1H), 6.52 (d, J=8.4 Hz, 1H), 4.36 (br t, J=8.5 Hz, 1H), 4.20-4.16 (m, 2H), 4.09 (br d, J=4.9 Hz, 1H), 3.85 (dd, J=4.6, 8.6 Hz, 2H), 3.64 (s, 3H), 2.79-2.66 (m, 2H), 2.24-2.19 (m, 2H).

### Step 7. Procedure for Preparation of spiro[3.3]heptan-2-ylmethyl (1-(3-chloro-4-(2,6-dioxopiperidin-3-yl)-2-methoxyphenyl)azetidin-3-yl)carbamate

To a solution of spiro[3.3]heptan-2-ylmethanol (20.0 mg, 158 umol, 1.00 eq) in tetrahydrofuran (1.00 mL) were added di(1H-imidazol-1-yl)methanone (51.4 mg, 317 umol, 2.00 eq) at 0° C. The reaction mixture was stirred at 25° C. for 1 h. The resulting solution was added to a mixture of 3-(4-(3-aminoazetidin-1-yl)-2-chloro-3-methoxyphenyl)piperidine-2,6-dione (45.0 mg, 139 umol, 1.00 eq) in tetrahydrofuran (0.500 mL) and dimethylformamide (0.500 mL) were added triethylamine (14.1 mg, 139 umol, 19.4 uL, 1.00 eq), 2,3,4,6,7,8,9,10-octahydropyrimido[1,2-a]azepine (21.2 mg, 139 umol, 21.0 uL, 1.00 eq). The reaction mixture was stirred at 25° C. for 16 h. Then, a solution of spiro[3.3]heptan-2-ylmethanol (20.0 mg, 158 umol, 1.00 eq) in tetrahydrofuran (1.00 mL) were added di(1H-imidazol-1-yl)methanone (51.4 mg, 317 umol, 2.00 eq), and the solution was added into the mixture. Finally, the reaction mixture was stirred at 25° C. for 48 h. The reaction mixture was filtered. The filtrate was purified by Prep-HPLC (column: Phenomenex luna C18 150*25 mm*10 um; mobile phase: [water (formic acid)-acetonitrile]; B %: 50%-80%, 10 min) and lyophilized to afford spiro[3.3]heptan-2-ylmethyl (1-(3-chloro-4-(2,6-dioxopiperidin-3-yl)-2-methoxyphenyl)azetidin-3-yl)carbamate (9.01 mg, 18.74 umol, 13% yield, 99% purity) as a white solid.

¹H NMR (400 MHz, DMSO-d₆) δ=10.82 (s, 1H), 7.77 (br d, J=7.5 Hz, 1H), 6.89 (d, J=8.4 Hz, 1H), 6.42 (d, J=8.5 Hz, 1H), 4.47-4.31 (m, 1H), 4.14 (br t, J=7.5 Hz, 2H), 4.05 (dd, J=5.0, 12.1 Hz, 1H), 3.89 (d, J=7.0 Hz, 2H), 3.70-3.59 (m, 5H), 2.78-2.69 (m, 1H), 2.49-2.43 (m, 1H), 2.40-2.31 (m, 1H), 2.27-2.15 (m, 1H), 2.06-1.92 (m, 5H), 1.91-1.84 (m, 2H), 1.80-1.66 (m, 4H). MS (ESI) m/z. 476.1 [M+H]⁺

### Example 63. Synthesis of Compound 64

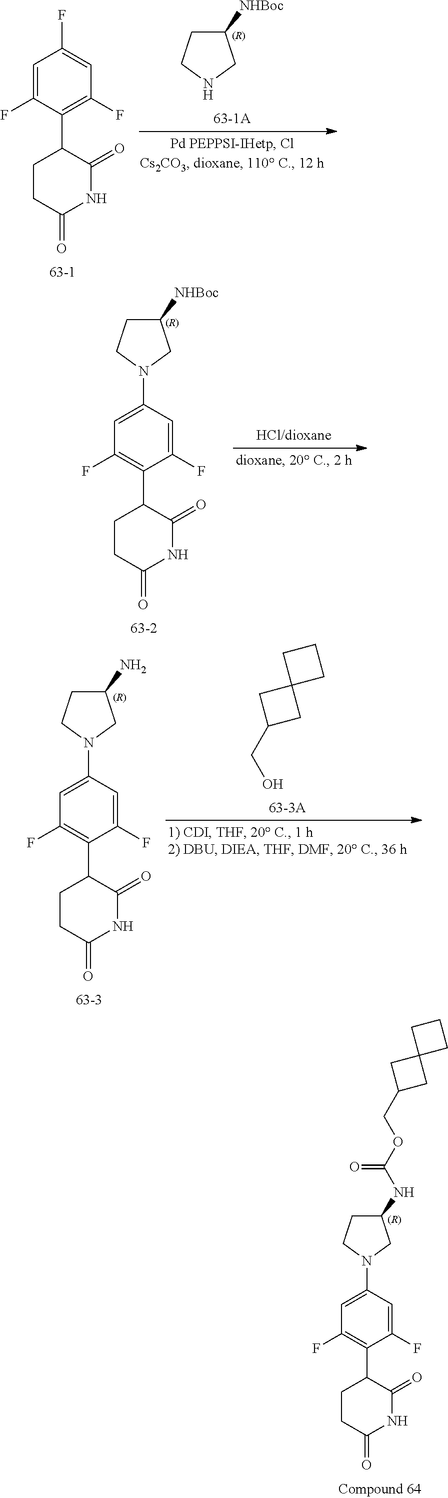

### Step 1. Procedure for Preparation of Compound 2—tert-butyl ((3R)-1-(4-(2,6-dioxopiperidin-3-yl)-3,5-difluorophenyl)pyrrolidin-3-yl)carbamate

To a solution of 3-(4-bromo-2,6-difluorophenyl)piperidine-2,6-dione (100 mg, 329 umol, 1.00 eq) in dioxane (2.00 mL) were added cesium carbonate (321 mg, 987 umol, 3.00 eq), (R)-tert-butyl pyrrolidin-3-ylcarbamate (91.9 mg, 493 umol, 1.50 eq) and [1,3-bis[2,6-bis(1-propylbutyl)phenyl]-4,5-dichloro-imidazol-2-ylidene]-dichloro-(3-chloropyridin-1-ium-1-yl)palladium (32.0 mg, 32.9 umol, 0.100 eq). The reaction mixture was stirred at 110° C. for 12 h. The reaction mixture was filtered and concentrated under reduced pressure to give a residue. The residue was purified by reverse phase chromatography (C18, 40 g; condition: water/acetonitrile=100:0 to 0:100, 0.1% formic acid) and lyophilized to afford tert-butyl ((3R)-1-(4-(2,6-dioxopiperidin-3-yl)-3,5-difluorophenyl)pyrrolidin-3-yl)carbamate (70.0 mg, 171 umol, 52% yield) as yellow oil.

¹H NMR (400 MHz, CDCl₃) δ=8.01 (br d, J=5.1 Hz, 1H), 6.10 (s, 1H), 6.07 (s, 1H), 4.71 (br s, 1H), 4.35 (br s, 1H), 3.95 (dd, J=5.1, 12.3 Hz, 1H), 3.55 (dd, J=6.1, 9.8 Hz, 1H), 3.42-3.34 (m, 1H), 3.34-3.26 (m, 1H), 3.12 (dd, J=4.0, 9.8 Hz, 1H), 2.85-2.75 (m, 1H), 2.73-2.61 (m, 1H), 2.39-2.24 (m, 2H), 2.19-2.09 (m, 1H), 1.99-1.91 (m, 1H), 1.46 (s, 9H).

### Step 2. Procedure for Preparation of Compound 3—3-(4-((R)-3-aminopyrrolidin-1-yl)-2,6-difluorophenyl)piperidine-2,6-dione

To a solution of tert-butyl ((3R)-1-(4-(2,6-dioxopiperidin-3-yl)-3,5-difluorophenyl)pyrrolidin-3-yl)carbamate (70.0 mg, 171 umol, 1.00 eq) in dioxane (1.00 mL) was added hydrochloric acid/dioxane (4.00 M, 1.75 mL, 40.9 eq). The reaction mixture was stirred at 20° C. for 2 h. The reaction mixture was concentrated under reduced pressure to afford 3-(4-((R)-3-aminopyrrolidin-1-yl)-2,6-difluorophenyl)piperidine-2,6-dione (59.0 mg, 171 umol, 99% yield, hydrochloride) as a yellow solid.

¹H NMR (400 MHz, DMSO-d₆) δ=10.85 (s, 1H), 8.45-8.16 (m, 2H), 6.29 (s, 1H), 6.26 (s, 1H), 4.04 (br dd, J=5.1, 12.7 Hz, 1H), 3.93 (br s, 1H), 3.55-3.49 (m, 1H), 3.47-3.39 (m, 1H), 3.35-3.24 (m, 2H), 2.80-2.75 (m, 1H), 2.53-2.52 (m, 1H), 2.33-2.25 (m, 1H), 2.15-2.02 (m, 2H), 1.98-1.90 (m, 1H).

### Step 3. Procedure for Preparation of spiro[3.3]heptan-2-ylmethyl ((3R)-1-(4-(2,6-dioxopiperidin-3-yl)-3,5-difluorophenyl)pyrrolidin-3-yl)carbamate

To a solution of spiro[3.4]octan-2-ylmethanol (25.0 mg, 198 umol, 1.00 eq) in tetrahydrofuran (0.300 mL) was added di(1H-imidazol-1-yl)methanone (48.2 mg, 297 umol, 1.50 eq). The reaction mixture was stirred at 20° C. for 1 h. The resulting solution was added to a mixture of 3-(4-((R)-3-aminopyrrolidin-1-yl)-2,6-difluorophenyl)piperidine-2,6-dione (59.0 mg, 171 umol, 99% yield, hydrochloride), 2,3,4,6,7,8,9,10-octahydropyrimido[1,2-a]azepine (26.0 mg, 171 umol, 25.7 uL, 1.00 eq) and N,N-diisopropylethylamine (22.1 mg, 171 umol, 29.7 uL, 1.00 eq) in dimethylformamide (0.500 mL). The reaction mixture was stirred at 20° C. for 16 h. The reaction mixture was concentrated under reduced pressure to give a residue. The residue was dissolved in dimethylformamide (1.5 mL) and then filtered. The filtrate was purified by Prep-HPLC (column: Phenomenex luna C18 150*25 mm*10 um; mobile phase: [water (formic acid)-acetonitrile]; B %: 52%-82%, 9 min) and lyophilized to afford spiro[3.3]heptan-2-ylmethyl ((3R)-1-(4-(2,6-dioxopiperidin-3-yl)-3,5-difluorophenyl)pyrrolidin-3-yl) carbamate (22.10 mg, 47.41 umol, 28% yield, 99% purity) as a white solid.

¹H NMR (400 MHz, DMSO-d₆) δ=10.83 (s, 1H), 7.48 (br d, J=6.5 Hz, 1H), 6.20 (s, 1H), 6.17 (s, 1H), 4.21-4.10 (m, 1H), 4.01 (br dd, J=5.1, 12.5 Hz, 1H), 3.89 (br d, J=6.9 Hz, 2H), 3.48-3.41 (m, 1H), 3.34 (br s, 1H), 3.26-3.18 (m, 1H), 3.04 (br dd, J=4.3, 9.4 Hz, 1H), 2.83-2.72 (m, 1H), 2.52 (br d, J=1.9 Hz, 1H), 2.43-2.34 (m, 1H), 2.18-2.11 (m, 1H), 2.10-1.99 (m, 3H), 1.99-1.89 (m, 4H), 1.86 (br d, J=7.5 Hz, 2H), 1.78-1.67 (m, 4H).

¹H NMR (400 MHz, DMSO-d₆, T=80° C.) δ=10.55 (br s, 1H), 7.16 (br d, J=3.6 Hz, 1H), 6.18 (s, 1H), 6.15 (s, 1H), 4.22-4.13 (m, 1H), 3.99 (dd, J=5.4, 12.4 Hz, 1H), 3.92 (d, J=6.8 Hz, 2H), 3.47 (dd, J=6.7, 9.8 Hz, 1H), 3.41-3.32 (m, 1H), 3.29-3.19 (m, 1H), 3.05 (br s, 1H), 2.84-2.71 (m, 1H), 2.54 (br d, J=3.8 Hz, 1H), 2.42-2.34 (m, 1H), 2.22-2.14 (m, 1H), 2.13-2.02 (m, 3H), 2.01-1.92 (m, 4H), 1.92-1.87 (m, 2H), 1.82-1.69 (m, 4H). MS (ESI) m/z 462.2 [M+H]⁺.

### Example 64. Synthesis of Compound 65

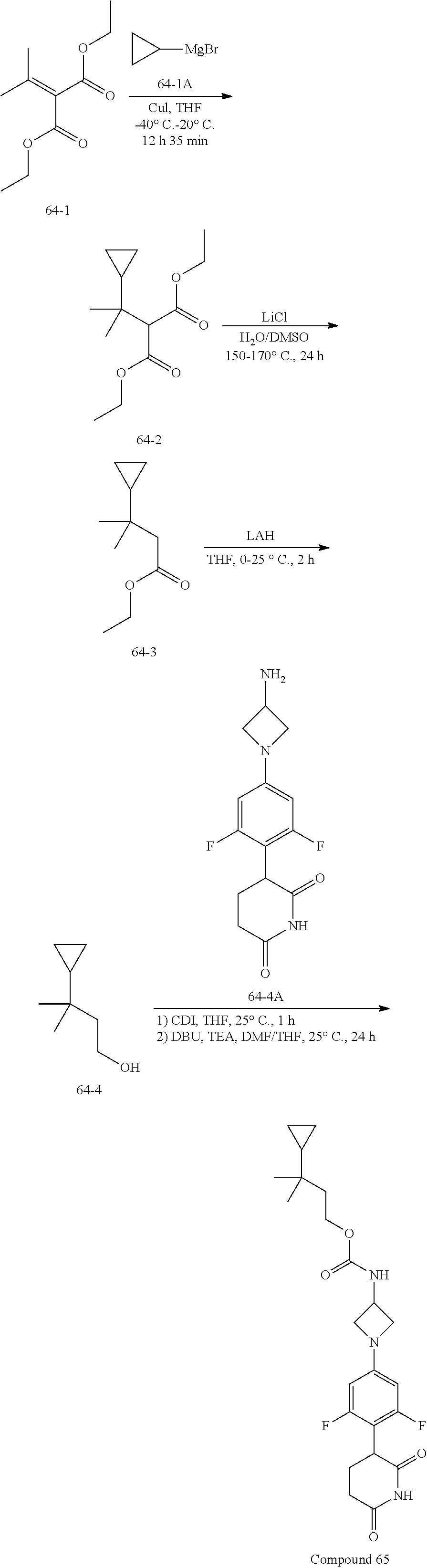

### Step 1. Procedure for Preparation of Compound 2—diethyl 2-(2-cyclopropylpropan-2-yl)malonate

To a solution of copper iodide (19.0 g, 100 mmol, 2.00 eq) in tetrahydrofuran (125 mL) at −40° C. was added bromo(cyclopropyl)magnesium (0.500, 400 mL, 4.00 eq) under nitrogen atmosphere. After stirring for 15 min, the suspension was allowed to warm up and stirred at 20° C. for 20 min before cooling back to −40° C. A solution of diethyl 2-(propan-2-ylidene) malonate (10.0 g, 50.0 mmol, 1.00 eq) in tetrahydrofuran (125 mL) was added, and the reaction was allowed to warm to 20° C. 12 h. The reaction mixture was then quenched with saturated sodium bicarbonate (50 mL) and filtered. The filtrate was extracted with ethyl acetate (3×125 mL). The combined extracts were dried over sodium sulfate and concentrated to get a residue. The residue was purified by column chromatography (SiO₂, petroleum ether/ethyl acetate=100/1 to 50/1) and concentrated under reduced pressure to afford diethyl 2-(1-cyclopropyl-1-methyl-ethyl)propanedioate (4.30 g, crude) as colorless oil.

¹H NMR (400 MHz, CDCl₃-d) δ=4.18 (q, J=7.1 Hz, 4H), 3.35 (s, 1H), 1.27 (t, J=7.1 Hz, 6H), 1.06-1.01 (m, 1H), 0.98 (s, 6H), 0.35-0.29 (m, 2H), 0.28-0.20 (m, 2H).

### Step 2. Procedure for Preparation of Compound 3—ethyl 3-cyclopropyl-3-methylbutanoate

To a solution of diethyl 2-(2-cyclopropylpropan-2-yl)malonate (1.00 g, 4.13 mmol, 1.00 eq) and lithium chloride (1.05 g, 24.8 mmol, 507 uL, 6.00 eq) in dimethylsulfoxide (25.0 mL) and water (0.300 mL). The mixture was stirred at 170° C. for 12 h. Then, the mixture was stirred at 150° C. for 12 h. The reaction mixture was quenched with water (20 mL) and extracted with methyl tert-butylether (3×30 mL). The combined organic layers were washed with brine (40 mL), dried over sodium sulfate, filtered and concentrated under reduced pressure to give a residue. The residue was purified by column chromatography (SiO₂, petroleum ether/ethyl acetate=100/1 to 50/1) and concentrated under reduced pressure to afford ethyl 3-cyclopropyl-3-methylbutanoate (190 mg, crude) as colorless oil.

¹H NMR (400 MHz, CDCl₃-d) δ=4.17-4.07 (m, 2H), 2.26 (s, 2H), 1.30-1.26 (m, 3H), 0.89 (s, 6H), 0.84-0.79 (m, 1H), 0.36-0.28 (m, 2H), 0.21 (br d, J=4.6 Hz, 2H).

### Step 3. Procedure for Preparation of Compound 4—3-cyclopropyl-3-methylbutan-1-ol

To a solution of ethyl 3-cyclopropyl-3-methylbutanoate (100 mg, 587 umol, 1.00 eq) in tetrahydrofuran (5.00 mL) was added lithium aluminium hydride (33.4 mg, 881 umol, 1.50 eq) at 0° C. The reaction mixture was stirred at 25° C. for 2 h. The reaction was quenched by addition of water (5 mL), 15% sodium hydroxide (5 mL) and water (15.00 mL). Then the mixture was filtered. The filtrate was extracted with ethyl acetate (3×15 mL). The combined organic layers were washed with brine (3×15 mL), dried over sodium sulfate, filtered and concentrated under reduced pressure to afford 3-cyclopropyl-3-methylbutan-1-ol (60.0 mg, 468 mol, 80% yield) as colorless oil.

¹H NMR (400 MHz, CDCl₃-d) δ=3.79 (t, J=7.5 Hz, 2H), 1.60 (t, J=7.5 Hz, 2H), 0.78 (s, 6H), 0.72-0.63 (m, 1H), 0.32-0.26 (m, 2H), 0.22-0.16 (m, 2H).

### Step 4. Procedure for Preparation of 3-cyclopropyl-3-methylbutyl (1-(4-(2,6-dioxopiperidin-3-yl)-3,5-difluorophenyl)azetidin-3-yl)carbamate

To a solution of 3-cyclopropyl-3-methylbutan-1-ol (10.0 mg, 78.0 umol, 1.00 eq) in tetrahydrofuran (1.00 mL) was added di(1H-imidazol-1-yl)methanone (19.0 mg, 117 umol, 1.50 eq). The mixture was stirred at 25° C. for 1 h. The resulting solution was added to a mixture of 3-(4-(3-aminoazetidin-1-yl)-2,6-difluorophenyl)piperidine-2,6-dione (31.9 mg, 77.8 umol, 1.00 eq, trifluoroacetate), 2,3,4,6,7,8,9,10-octahydropyrimido[1,2-a]azepine (11.9 mg, 77.8 umol, 11.7 uL, 1.00 eq) and triethylamine (7.88 mg, 77.8 umol, 10.8 uL, 1.00 eq) in dimethylformamide (0.500 mL) and tetrahydrofuran (0.500 mL). The reaction mixture was stirred at 25° C. for 24 h. The reaction mixture was filtered. The filtrate was purified by Prep-HPLC (column: Phenomenex luna C18 150*25 mm*10 um; mobile phase: [water (formic acid)-acetonitrile]; B %: 50%-80%, 8 min) and lyophilized to afford 3-cyclopropyl-3-methylbutyl (1-(4-(2,6-dioxopiperidin-3-yl)-3,5-difluorophenyl)azetidin-3-yl)carbamate (5.49 mg, 11.85 umol, 15% yield, 97% purity) as a white solid.

¹H NMR (400 MHz, DMSO-d₆) δ=10.85 (s, 1H), 7.76 (br d, J=7.0 Hz, 1H), 6.14 (d, J=11.1 Hz, 2H), 4.51-4.32 (m, 1H), 4.07 (br s, 5H), 3.63 (br t, J=6.7 Hz, 2H), 2.83-2.69 (m, 1H), 2.47 (br s, 1H), 2.14-2.02 (m, 1H), 1.99-1.88 (m, 1H), 1.54 (br t, J=7.6 Hz, 2H), 0.74 (s, 6H), 0.71-0.61 (m, 1H), 0.27-0.21 (m, 2H), 0.18-0.12 (m, 2H). MS (ESI) m/z. 472.0 [M+Na]⁺

### Example 65. Synthesis of Compound 66

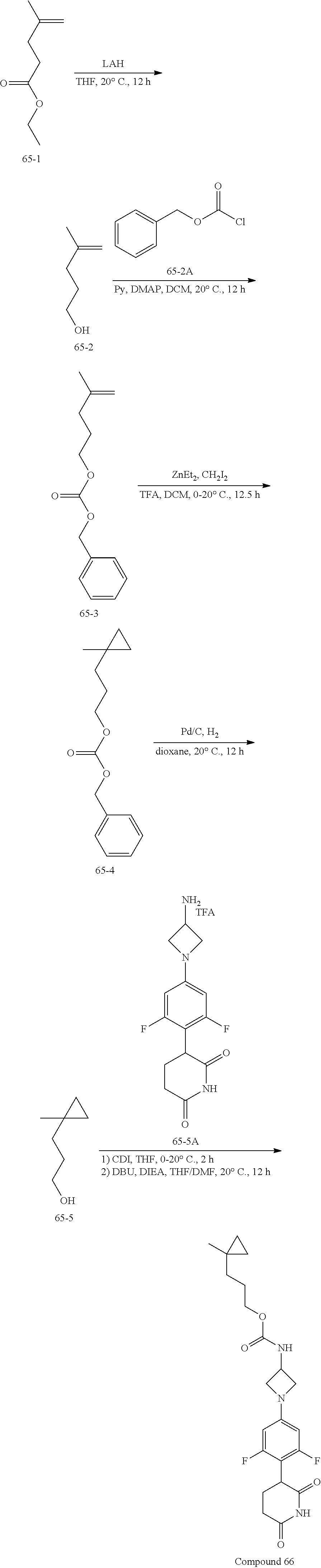

### Step 1. Procedure for Preparation of Compound 2—4-methylpent-4-en-1-ol

To a solution of ethyl 4-methylpent-4-enoate (2.00 g, 14.1 mmol, 1.00 eq) in tetrahydrofuran (20.0 mL) was added lithium aluminum hydride (1.07 g, 28.1 mmol, 2.00 eq) at 0° C. The reaction mixture was stirred at 20° C. for 12 h. The reaction mixture was quenched with water (1.07 mL), 15% sodium hydroxide (1.07 mL), water (3.21 mL), then filtered and the filtrate was concentrated under reduced pressure to afford 4-methylpent-4-en-1-ol (600 mg, 5.99 mmol, 43% yield) as yellow oil.

¹H NMR (400 MHz, CDCl₃-d) δ=4.75-4.70 (m, 2H), 3.67 (t, J=6.5 Hz, 2H), 2.11 (t, J=7.6 Hz, 2H), 1.75 (s, 3H), 1.74-1.67 (m, 2H).

### Step 2. Procedure for Preparation of Compound 3—benzyl (4-methylpent-4-en-1-yl) carbonate

To a solution of 4-methylpent-4-en-1-ol (500 mg, 4.99 mmol, 1.00 eq) in dichloromethane (5.00 mL) were added pyridine (790 mg, 9.98 mmol, 806 uL, 2.00 eq), 4-dimethylaminopyridine (30.5 mg, 250 umol, 0.0500 eq) and benzyl carbonochloridate (937 mg, 5.49 mmol, 781 uL, 1.10 eq). The reaction mixture was stirred at 20° C. for 12 h. The reaction mixture was filtered and the filtrate was concentrated under reduced pressure to give a residue. The residue was purified by column chromatography (SiO₂, petroleum ether/ethyl acetate=1/0=to 50/1) to afford benzyl (4-methylpent-4-en-1-yl) carbonate (600 mg, 2.56 mmol, 51% yield) as yellow oil.

¹H NMR (400 MHz, CDCl₃-d) δ=7.44-7.30 (m, 5H), 5.17 (s, 2H), 4.75 (s, 1H), 4.70 (d, J=1.0 Hz, 1H), 4.17 (t, J=6.6 Hz, 2H), 2.10 (t, J=7.6 Hz, 2H), 1.87-1.79 (m, 2H), 1.73 (s, 3H).

### Step 3. Procedure for Preparation of Compound 4—benzyl (3-(J-methylcyclopropyl)propyl) carbonate

To a solution of diethylzinc (1.00 M, 1.28 mL, 3.00 eq) in dichloromethane (1.00 mL) was dropwise added trifluoroacetic acid (146 mg, 1.28 mmol, 94.8 uL, 3.00 eq) in dichloromethane (1.00 mL) at 0° C. for 25 min under nitrogen atmosphere. To this suspension was dropwise added diiodomethane (343 mg, 1.28 mmol, 103 uL, 3.00 eq) in dichloromethane (1.00 mL) at 0° C. for 10 min under nitrogen atmosphere. The resulting solution was stirred at 0° C. for 25 min, at which time benzyl 4-methylpent-4-enyl carbonate (100 mg, 427 umol, 1.00 eq) was added. The reaction mixture was stirred at 0° C. for 30 min and then warmed to 20° C. for 11 h. The reaction mixture was added saturated aqueous ammonium chloride solution (2.00 mL) to quench diethylzinc, and then the mixture was extracted with ethyl acetate (3×3 mL). The combined organic phases were dried with anhydrous sodium sulfate, filtered and concentrated under reduced pressure to afford benzyl (3-(1-methylcyclopropyl)propyl) carbonate (100 mg, 403 umol, 94% yield) as yellow oil.

¹H NMR (400 MHz, DMSO-d₆) δ=7.42-7.30 (m, 5H), 5.12 (s, 2H), 4.09 (t, J=6.7 Hz, 2H), 1.69-1.63 (m, 2H), 1.25-1.21 (m, 2H), 0.98 (s, 3H), 0.21 (d, J=2.4 Hz, 4H).

### Step 4. Procedure for Preparation of Compound 5—3-(1-methylcyclopropyl)propan-1-ol

To a solution of benzyl 3-(1-methylcyclopropyl)propyl carbonate (100 mg, 403 umol, 1.00 eq) in dioxane (1.00 mL) was added palladium on activated carbon (100 mg, 10% purity). The reaction mixture was stirred at 20° C. for 12 h under 15 psi of hydrogen atmosphere. The reaction mixture was filtered and the filtrate was concentrated under reduced pressure to afford 3-(1-methylcyclopropyl)propan-1-ol (30.0 mg, 263 umol, 65% yield) as yellow oil.

¹H NMR (400 MHz, CDCl₃-d) δ=3.66 (t, J=6.6 Hz, 2H), 1.70-1.64 (m, 2H), 1.31-1.29 (m, 2H), 1.04 (s, 3H), 0.25 (br d, J=4.8 Hz, 4H).

### Step 5. Procedure for Preparation of 3-(1-methylcyclopropyl)propyl (1-(4-(2,6-dioxopiperidin-3-yl)-3,5-difluorophenyl)azetidin-3-yl)carbamate

To a solution of 3-(1-methylcyclopropyl)propan-1-ol (30.0 mg, 263 umol, 1.00 eq) in tetrahydrofuran (0.300 mL) was added di(1H-imidazol-1-yl)methanone (63.9 mg, 394 umol, 1.50 eq) at 0° C. The mixture was stirred at 20° C. for 2 h. The resulting solution was added to a mixture of 3-(4-(3-aminoazetidin-1-yl)-2,6-difluorophenyl)piperidine-2,6-dione (85.0 mg, 208 umol, 1.00 eq, trifluoroacetate), 2,3,4,6,7,8,9,10-octahydropyrimido[1,2-a]azepine (31.6 mg, 208 umol, 31.3 uL, 1.00 eq) and N,N-diisopropylethylamine (40.3 mg, 312 umol, 54.3 uL, 1.50 eq) in tetrahydrofuran (0.300 mL) and dimethylformamide (0.300 mL). The reaction mixture was stirred at 20° C. for 12 h. The reaction mixture was concentrated under reduced pressure to give a residue. The residue was diluted with dimethylformamide (1.00 mL) and then filtered. The filtrate was purified by Prep-HPLC (column: Phenomenex luna C18 150*25 mm*10 um; mobile phase: [water (formic acid)-acetonitrile]; B %: 47%-77%, 9 min) and lyophilized to afford 3-(1-methylcyclopropyl)propyl (1-(4-(2,6-dioxopiperidin-3-yl)-3,5-difluorophenyl)azetidin-3-yl)carbamate (21.16 mg, 48.11 umol, 23% yield, 99% purity) as a white solid.

¹H NMR (400 MHz, DMSO-d₆) δ=10.85 (s, 1H), 7.79 (br d, J=6.6 Hz, 1H), 6.15 (s, 1H), 6.13 (s, 1H), 4.48-4.33 (m, 1H), 4.08 (br t, J=7.6 Hz, 2H), 4.05-4.00 (m, 1H), 3.95 (br t, J=6.4 Hz, 2H), 3.62 (br t, J=6.4 Hz, 2H), 2.82-2.73 (m, 1H), 2.56 (br d, J=5.1 Hz, 1H), 2.12-2.05 (m, 1H), 2.03-1.92 (m, 1H), 1.66-1.58 (m, 2H), 1.27-1.20 (m, 2H), 0.99 (s, 3H), 0.22 (br d, J=2.4 Hz, 4H). MS (ESI) m/z 436.0 [M+H]⁺

### Example 66. Synthesis of Compound 67

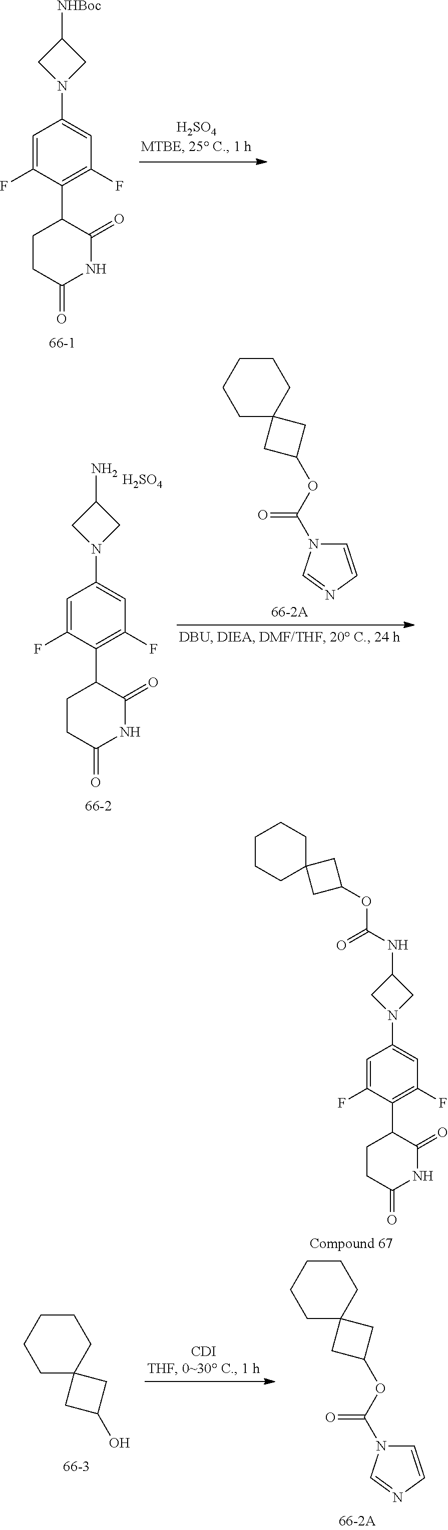

### Step 1. Procedure for Preparation of Compound 2—3-(4-(3-aminoazetidin-1-yl)-2,6-difluorophenyl)piperidine-2,6-dione

A solution of tert-butyl (1-(4-(2,6-dioxopiperidin-3-yl)-3,5-difluorophenyl)azetidin-3-yl)carbamate (200 mg, 505 umol, 1.00 eq) in methyl tert-butyl ether (1.00 mL) was added into a mixture of sulfuric acid (151 mg, 1.52 mmol, 82.5 uL, 98% purity, 3.00 eq) and methyl tert-butyl ether (2.00 mL). The mixture was stirred at 25° C. for 1 h. The mixture was diluted with acetonitrile (15 mL) and stirred for 15 min. Then the mixture was filtered and the filter cake was concentrated under reduced pressure to afford 3-(4-(3-aminoazetidin-1-yl)-2,6-difluorophenyl)piperidine-2,6-dione (145 mg, 258 umol, 51% yield, 70% purity, sulfate salt) as a yellow solid.

¹H NMR (400 MHz, DMSO-d₆) δ=10.88-10.83 (m, 1H), 8.30 (br s, 4H), 6.27 (br d, J=11.1 Hz, 2H), 4.11 (br s, 2H), 4.06 (br d, J=5.6 Hz, 1H), 3.92 (br dd, J=6.9, 11.3 Hz, 1H), 3.81 (br d, J=3.9 Hz, 2H), 2.84-2.72 (m, 2H), 1.99-1.88 (m, 2H).

### Step 2. Procedure for Preparation of Compound 2A—spiro[3.5]nonan-2-yl 1H-imidazole-1-carboxylate

To a solution of spiro[3.5]nonan-2-ol (40.0 mg, 285 umol, 1.00 eq) in tetrahydrofuran (1.00 mL) was added di(1H-imidazol-1-yl)methanone (37.0 mg, 228 umol, 0.800 eq) at 0° C. The mixture was stirred at 20° C. for 1 h. The mixture was used directly to the next step.

### Step 3. Procedure for Preparation of spiro[3.5]nonan-2-yl (1-(4-(2,6-dioxopiperidin-3-yl)-3,5-difluorophenyl)azetidin-3-yl)carbamate

To a solution of 3-(4-(3-aminoazetidin-1-yl)-2,6-difluorophenyl)piperidine-2,6-dione (143 mg, 256 umol, 70% purity, 1.00 eq, sulfate salt) in tetrahydrofuran (1.00 mL) and dimethylformamide (1.00 mL) was added 2,3,4,6,7,8,9,10-octahydropyrimido[1,2-a]azepine (38.9 mg, 256 umol, 38.6 uL, 1.00 eq) and N,N-diisopropylethylamine (33.1 mg, 256 umol, 44.6 uL, 1.00 eq). The mixture was stirred at 20° C. for 0.5 h. Then spiro[3.5]nonan-2-yl 1H-imidazole-1-carboxylate (60.0 mg, 256 umol, 1.00 eq) was added into the mixture. The mixture was stirred at 20° C. for 11.5 h. N,N-diisopropylethylamine (66.2 mg, 512 umol, 89.2 uL, 2.00 eq) was added into the mixture. The mixture was stirred at 20° C. for 12 h. The mixture was concentrated under reduced pressure to give a residue. The residue was purified by Prep-HPLC (column: Phenomenex luna C18 150*25 mm*10 um; mobile phase: [water (formic acid)-acetonitrile]; B %: 53%-83%, 10 min) and lyophilized to afford spiro[3.5]nonan-2-yl (1-(4-(2,6-dioxopiperidin-3-yl)-3,5-difluorophenyl)azetidin-3-yl)carbamate (6.67 mg, 14.31 umol, 5% yield, 99% purity) as an off-white solid.

¹H NMR (400 MHz, DMSO-d₆) δ=10.85 (s, 1H), 7.82 (br d, J=7.3 Hz, 1H), 6.14 (d, J=11.1 Hz, 2H), 4.82 (quin, J=7.2 Hz, 1H), 4.43-4.34 (m, 1H), 4.08 (br t, J=7.8 Hz, 2H), 4.05-4.00 (m, 1H), 3.61 (br t, J=6.8 Hz, 2H), 2.83-2.72 (m, 1H), 2.47 (br s, 1H), 2.20-2.13 (m, 2H), 2.10-2.00 (m, 1H), 1.99-1.88 (m, 1H), 1.67-1.60 (m, 2H), 1.43-1.29 (m, 10H). MS (ESI) m/z 462.1 [M+H]⁺

### Example 67. Synthesis of Compound 68

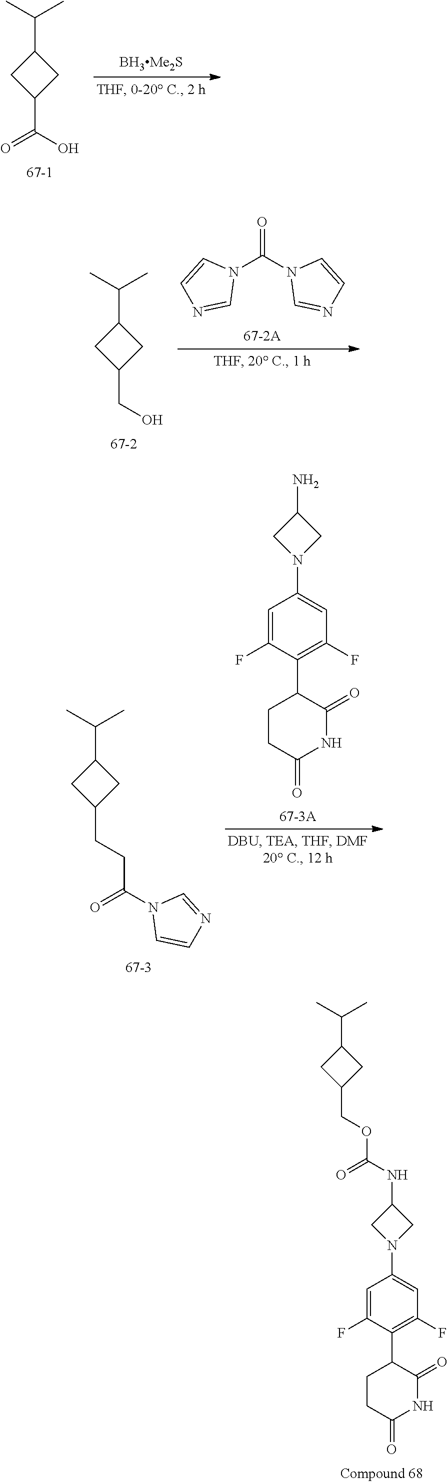

### Step 1. Procedure for Preparation of Compound 2—(3-isopropylcyclobutyl)methanol

To a solution of 3-isopropylcyclobutanecarboxylic acid (200 mg, 1.41 mmol, 1.00 eq) in tetrahydrofuran (2.00 mL) was added borane dimethyl sulfide complex (10.0 M, 281 uL, 2.00 eq) at 0° C. The reaction mixture was stirred at 20° C. for 2 h. The reaction mixture was quenched by methanol (20 mL) and concentrated under reduced pressure to afford (3-isopropylcyclobutyl)methanol (160 mg, crude) as a white solid.

¹H NMR (400 MHz, CDCl₃-d) δ=3.66 (d, J=7.5 Hz, 1H), 3.52 (d, J=6.4 Hz, 1H), 2.35-2.21 (m, 1H), 2.15-2.03 (m, 1H), 1.97-1.81 (m, 1H), 1.80-1.76 (m, 2H), 1.55-1.41 (m, 1H), 1.40-1.16 (m, 2H), 0.79 (t, J=6.8 Hz, 6H).

### Step 2. Procedure for Preparation of Compound 3—(3-isopropylcyclobutyl)methyl 1H-imidazole-1-carboxylate

To a solution of (3-isopropylcyclobutyl)methanol (50.0 mg, 390 umol, 1.00 eq) in tetrahydrofuran (0.500 mL) was added di(1H-imidazol-1-yl)methanone (95.0 mg, 585 umol, 1.50 eq). The reaction mixture was stirred at 20° C. for 1 h. The reaction mixture was concentrated under reduced pressure to (3-isopropylcyclobutyl)methyl 1H-imidazole-1-carboxylate (86.0 mg, crude) which was used into next step directly.

### Step 3. Procedure for Preparation of (3-isopropylcyclobutyl)methyl (1-(4-(2,6-dioxopiperidin-3-yl)-3,5-difluorophenyl)azetidin-3-yl)carbamate

To a solution of 3-(4-(3-aminoazetidin-1-yl)-2,6-difluorophenyl)piperidine-2,6-dione (70.3 mg, 227 umol, 1.00 eq, hydrochloride) in tetrahydrofuran (1.00 mL) and dimethylformamide (1.00 mL), then added 2,3,4,6,7,8,9,10-octahydropyrimido[1,2-a]azepine (58.9 mg, 387 umol, 58.3 uL, 1.00 eq), triethylamine (39.2 mg, 387 umol, 53.9 uL, 1.00 eq) and (3-isopropylcyclobutyl)methyl 1H-imidazole-1-carboxylate (55.0 mg, 227 umol, 1.00 eq). The reaction mixture was stirred at 20° C. for 12 h. The reaction mixture was concentrated under reduced pressure to give a residue. The residue was diluted with dimethylformamide (1 mL) and then filtered. The filtrate was purified by Prep-HPLC (column: Phenomenex luna C18 150*25 mm*10 um; mobile phase: [water (formic acid)-acetonitrile]; B %: 52%-82%, 9 min) and lyophilized to afford (3-isopropylcyclobutyl)methyl (1-(4-(2,6-dioxopiperidin-3-yl)-3,5-difluorophenyl)azetidin-3-yl)carbamate (22.00 mg, 48.45 umol, 13% yield, 99% purity) as a white solid.

¹H NMR (400 MHz, DMSO-d₆) δ=10.86 (s, 1H), 7.83 (br t, J=6.6 Hz, 1H), 6.15 (s, 1H), 6.13 (s, 1H), 4.41 (br dd, J=2.2, 4.8 Hz, 1H), 4.08 (t, J=7.7 Hz, 2H), 4.05-4.00 (m, 2H), 3.86 (d, J=6.3 Hz, 1H), 3.62 (t, J=6.8 Hz, 2H), 2.82-2.73 (m, 1H), 2.54 (br d, J=1.4 Hz, 1H), 2.40-2.33 (m, 1H), 2.05 (br d, J=4.0 Hz, 1H), 2.03-1.99 (m, 1H), 1.99-1.89 (m, 2H), 1.76-1.72 (m, 2H), 1.53-1.38 (m, 1H), 1.37-1.32 (m, 1H), 0.76 (t, J=7.3 Hz, 6H). MS (ESI) m/z 450.3 [M+H]⁺

### Example 68. Synthesis of Compound 69

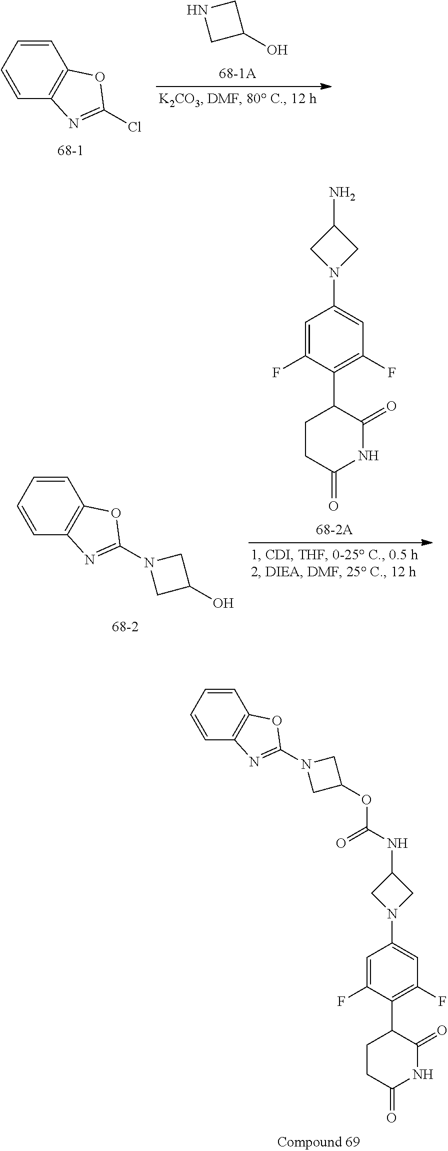 [SMILES: Clc1nc2ccccc2o1 ; OC1CNC1 ; OC1CN(c2nc3ccccc3o2)C1 ; NC1CN(c2cc(F)c(C3CCC(=O)NC3=O)c(F)c2)C1 ; O=C1CCC(c2c(F)cc(N3CC(NC(=O)OC4CN(c5nc6ccccc6o5)C4)C3)cc2F)C(=O)N1]

### Step 1. Procedure for Preparation of Compound 2—1-(benzo[d]oxazol-2-yl)azetidin-3-ol

To a solution of 2-chloro-1,3-benzoxazole (500 mg, 3.26 mmol, 370 uL, 1.00 eq) and azetidin-3-ol (428 mg, 3.91 mmol, 1.20 eq, mesylate) in dimethylformamide (5.00 mL) was added potassium carbonate (450 mg, 3.26 mmol, 1.00 eq). The mixture was stirred at 80° C. for 12 h. The reaction mixture was diluted with water (30 mL) and exacted with ethyl acetate (3×30 mL). The organic phase was washed with brine (2×10 mL), dried over sodium sulfate, filtered and concentrated under reduced pressure to give a residue. The residue was purified by column chromatography (SiO₂, petroleum ether/ethyl acetate=1/1 to 0/1) to afford 1-(benzo[d]oxazol-2-yl)azetidin-3-ol (600 mg, 3.15 mmol, 97% yield) as a yellow solid.

¹H NMR (400 MHz, DMSO-d₆) δ=7.42 (d, J=8.0 Hz, 1H), 7.32 (d, J=7.8 Hz, 1H), 7.17 (t, J=7.6 Hz, 1H), 7.06 (d, J=7.6 Hz, 1H), 5.90 (d, J=6.6 Hz, 1H), 4.70-4.63 (m, 1H), 4.40 (t, J=7.6 Hz, 2H), 3.98 (dd, J=4.8, 8.6 Hz, 2H).

### Step 2. Procedure for Preparation of 1-(benzo[d]oxazol-2-yl)azetidin-3-yl (1-(4-(2,6-dioxopiperidin-3-yl)-3,5-difluorophenyl)azetidin-3-yl)carbamate

To a solution of 1-(benzo[d]oxazol-2-yl)azetidin-3-ol (80.0 mg, 421 umol, 1.00 eq) in tetrahydrofuran (1.00 mL) was added 1,1′-carbonyldiimidazole (68.2 mg, 421 umol, 1.00 eq) at 0° C. The mixture was stirred at 25° C. for 0.5 h to give a resulting solution. A solution of 3-(4-(3-aminoazetidin-1-yl)-2,6-difluorophenyl)piperidine-2,6-dione (165 mg, 422 umol, 1.00 eq, mesylate), 2,3,4,6,7,8,9,10-octahydropyrimido[1,2-a]azepine (64.3 mg, 422 umol, 63.6 uL, 1.00 eq) and N,N-diisopropylethylamine (54.6 mg, 422 umol, 73.5 uL, 1.00 eq) in dimethylformamide (1.00 mL) was added into the resulting solution. The mixture was stirred at 25° C. for 12 h. The mixture was filtered to give filtrate. The filtrate was purified by Prep-HPLC (column: Phenomenex luna C18 150*25 mm*10 um; mobile phase: [water(formic acid)-acetonitrile]; B %: 33%-63%, 9 min) and lyophilized in vacuo to afford 1-(benzo[d]oxazol-2-yl)azetidin-3-yl (1-(4-(2,6-dioxopiperidin-3-yl)-3,5-difluorophenyl)azetidin-3-yl)carbamate (26.07 mg, 49.4 umol, 12% yield, 97% purity) as a white solid.

¹H NMR (400 MHz, DMSO-d₆) δ=10.86 (s, 1H), 8.20 (br d, J=7.0 Hz, 1H), 7.43 (d, J=7.8 Hz, 1H), 7.33 (d, J=7.8 Hz, 1H), 7.18 (t, J=7.6 Hz, 1H), 7.10-7.03 (m, 1H), 6.16 (d, J=11.0 Hz, 2H), 5.32-5.23 (m, 1H), 4.52 (dd, J=7.0, 9.2 Hz, 2H), 4.49-4.42 (m, 1H), 4.16-4.09 (m, 4H), 4.04 (br dd, J=5.1, 12.6 Hz, 1H), 3.67 (br t, J=6.8 Hz, 2H), 2.84-2.74 (m, 1H), 2.49-2.40 (m, 1H), 2.12-2.05 (m, 1H), 2.00-1.90 (m, 1H). MS (ESI) m/z 512.1 [M+H]⁺

### Example 69. Synthesis of Compound 70

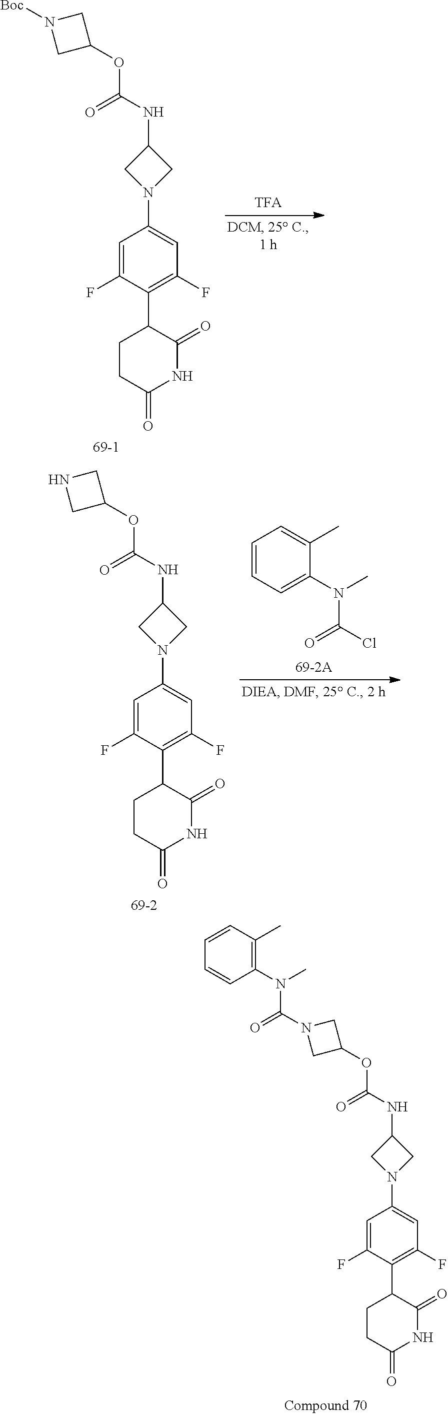 [SMILES: CC(C)(C)OC(=O)N1CC(OC(=O)NC2CN(c3cc(F)c(C4CCC(=O)NC4=O)c(F)c3)C2)C1 ; O=C1CCC(c2c(F)cc(N3CC(NC(=O)OC4CNC4)C3)cc2F)C(=O)N1 ; Cc1ccccc1N(C)C(=O)Cl ; Cc1ccccc1N(C)C(=O)N1CC(OC(=O)NC2CN(c3cc(F)c(C4CCC(=O)NC4=O)c(F)c3)C2)C1]

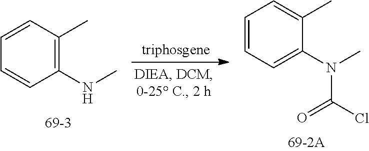

### Step 1. Procedure for Compound 2—azetidin-3-yl (1-(4-(2,6-dioxopiperidin-3-yl)-3,5-difluorophenyl)azetidin-3-yl)carbamate

To a solution of tert-butyl 3-(((1-(4-(2,6-dioxopiperidin-3-yl)-3,5-difluorophenyl)azetidin-3-yl)carbamoyl)oxy)azetidine-1-carboxylate (100 mg, 202 umol, 1.00 eq) in dichloromethane (5.00 mL) was added trifluoroacetic acid (1.54 g, 13.5 mmol, 1.00 mL, 66.8 eq). The mixture was stirred at 25° C. for 1 h. The mixture was concentrated under reduced pressure to give azetidin-3-yl (1-(4-(2,6-dioxopiperidin-3-yl)-3,5-difluorophenyl)azetidin-3-yl)carbamate (70.0 mg, crude) as colorless oil. MS (ESI) m/z 395.0 [M+H]⁺

### Step 2. Procedure for Preparation of Compound 2A—methyl(o-tolyl)carbamic chloride

To a solution of N, 2-dimethylaniline (30.0 mg, 248 umol, 30.6 uL, 1.00 eq) in dichloromethane (2.00 mL) were added N,N-diisopropylethylamine (96.0 mg, 743 umol, 129 uL, 3.00 eq) and bis(trichloromethyl) carbonate (88.2 mg, 297 umol, 1.20 eq) at 0° C. The mixture was stirred at 25° C. for 2 h. The mixture was concentrated under reduced pressure to give methyl(o-tolyl)carbamic chloride (40.0 mg, crude) as colorless oil.

### Step 3. Procedure for 1-(methyl(o-tolyl)carbamoyl)azetidin-3-yl (1-(4-(2,6-dioxopiperidin-3-yl)-3,5-difluorophenyl)azetidin-3-yl)carbamate

To a solution of azetidin-3-yl (1-(4-(2,6-dioxopiperidin-3-yl)-3,5-difluorophenyl)azetidin-3-yl)carbamate (70.0 mg, 178 umol, 1.00 eq) in dichloromethane (2.00 mL) were added N,N-diisopropylethylamine (68.8 mg, 533 umol, 92.8 uL, 3.00 eq) and methyl(o-tolyl)carbamic chloride (39.1 mg, 213 umol, 1.20 eq). The mixture was stirred at 25° C. for 2 h. The mixture was concentrated under reduced pressure to give a residue. The residue was purified by Prep-HPLC (column: Phenomenex luna C18 150*25 mm*10 um; mobile phase: [water(formic acid)-acetonitrile]; B %: 62%-92%, 9 min) and lyophilized to afford 1-(methyl(o-tolyl)carbamoyl)azetidin-3-yl (1-(4-(2,6-dioxopiperidin-3-yl)-3,5-difluorophenyl)azetidin-3-yl)carbamate (31.22 mg, 55.92 umol, 31.50% yield, 97% purity) as an yellow solid.

¹H NMR (400 MHz, DMSO-d₆) δ=10.85 (s, 1H), 8.03 (br d, J=7.5 Hz, 1H), 7.29 (br d, J=2.8 Hz, 1H), 7.23 (br d, J=3.3 Hz, 3H), 6.13 (br d, J=11.3 Hz, 2H), 4.77 (br d, J=3.1 Hz, 1H), 4.40-4.28 (m, 1H), 4.10-3.99 (m, 3H), 3.65 (br t, J=7.6 Hz, 2H), 3.58 (br t, J=6.5 Hz, 2H), 3.28-3.18 (m, 2H), 3.01 (s, 3H), 2.82-2.72 (m, 1H), 2.60-2.53 (m, 1H), 2.19 (s, 3H), 2.12-2.00 (m, 1H), 1.98-1.88 (m, 1H). MS (ESI) m/z 542.3 [M+H]⁺

### Example 70. Synthesis of Compound 71

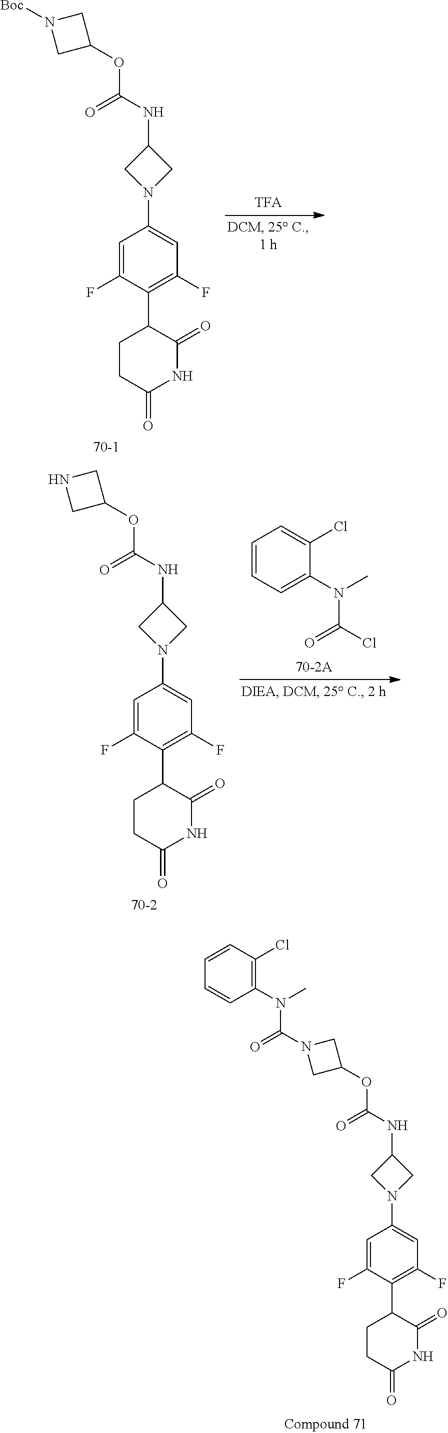 [SMILES: CC(C)(C)OC(=O)N1CC(OC(=O)NC2CN(c3cc(F)c(C4CCC(=O)NC4=O)c(F)c3)C2)C1 ; O=C1CCC(c2c(F)cc(N3CC(NC(=O)OC4CNC4)C3)cc2F)C(=O)N1 ; CN(C(=O)Cl)c1ccccc1Cl ; CN(C(=O)N1CC(OC(=O)NC2CN(c3cc(F)c(C4CCC(=O)NC4=O)c(F)c3)C2)C1)c1ccccc1Cl]

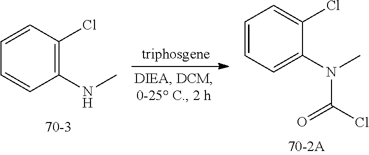

### Step 1. Procedure for Compound 2—azetidin-3-yl (1-(4-(2,6-dioxopiperidin-3-yl)-3,5-difluorophenyl)azetidin-3-yl)carbamate

To a solution of tert-butyl 3-(((1-(4-(2,6-dioxopiperidin-3-yl)-3,5-difluorophenyl)azetidin-3-yl)carbamoyl)oxy)azetidine-1-carboxylate (100 mg, 202 umol, 1.00 eq) in dichloromethane (5.00 mL) was added trifluoroacetic acid (1.54 g, 13.5 mmol, 1.00 mL, 66.8 eq). The mixture was stirred at 25° C. for 1 h. The mixture was concentrated under reduced pressure to give azetidin-3-yl (1-(4-(2,6-dioxopiperidin-3-yl)-3,5-difluorophenyl)azetidin-3-yl)carbamate (70.0 mg, crude) as colorless oil. MS (ESI) m/z 394.9 [M+H]⁺

### Step 2. Procedure for Preparation of Compound 2A—(2-chlorophenyl)(methyl)carbamic chloride

To a solution of 2-chloro-N-methylaniline (45.0 mg, 318 umol, 1.00 eq) in dichloromethane (2.00 mL) was added N,N-diisopropylethylamine (123 mg, 953 umol, 166 uL, 3.00 eq) and bis(trichloromethyl) carbonate (123 mg, 413 umol, 1.30 eq) at 0° C. The mixture was stirred at 25° C. for 1 h. The mixture was concentrated under reduced pressure to give (2-chlorophenyl)(methyl)carbamic chloride (60 mg, crude) as colorless oil.

### Step 3. Procedure for 1-((2-chlorophenyl)(methyl)carbamoyl)azetidin-3-yl (1-(4-(2,6-dioxopiperidin-3-yl)-3,5-difluorophenyl)azetidin-3-yl)carbamate

To a solution of azetidin-3-yl (1-(4-(2,6-dioxopiperidin-3-yl)-3,5-difluorophenyl)azetidin-3-yl)carbamate (70.0 mg, 178 umol, 1.00 eq) in dichloromethane (2.00 mL) was added N,N-diisopropylethylamine (68.8 mg, 532 umol, 92.8 uL, 3.00 eq) and (2-chlorophenyl)(methyl)carbamic chloride (43.5 mg, 213 umol, 1.20 eq). The mixture was stirred at 25° C. for 2 h. The mixture was concentrated under reduced pressure to give a residue. The residue was purified by reverse phase chromatography (C18, 40 g; condition: water/acetonitrile=100:0 to 0:100, 0.1% formic acid) and lyophilized to give a crude product. The crude product was purified by Prep-HPLC (column: Phenomenex luna C18 150*25 mm*10 um; mobile phase: [water(formic acid)-acetonitrile]; B %: 62%-92%, 9 min) and lyophilized to afford 1-((2-chlorophenyl)(methyl)carbamoyl)azetidin-3-yl (1-(4-(2,6-dioxopiperidin-3-yl)-3,5-difluorophenyl)azetidin-3-yl)carbamate (18.37 mg, 32.36 umol, 18.23% yield, 99% purity) as an off-white solid.

¹H NMR (400 MHz, DMSO-d₆) δ=10.86 (s, 1H), 8.06 (br d, J=7.6 Hz, 1H), 7.59 (br d, J=7.5 Hz, 1H), 7.49-7.34 (m, 3H), 6.14 (br d, J=11.1 Hz, 2H), 4.86-4.78 (m, 1H), 4.42-4.30 (m, 1H), 4.10-4.04 (m, 2H), 4.04-3.98 (m, 1H), 3.72 (br t, J=8.1 Hz, 2H), 3.59 (br t, J=6.5 Hz, 2H), 3.30 (br s, 2H), 3.04 (s, 3H), 2.84-2.72 (m, 1H), 2.60-2.54 (m, 1H), 2.14-2.01 (m, 1H), 1.99-1.89 (m, 1H). MS (ESI) m/z 562.2 [M+H]⁺

### Example 71. Synthesis of Compound 72

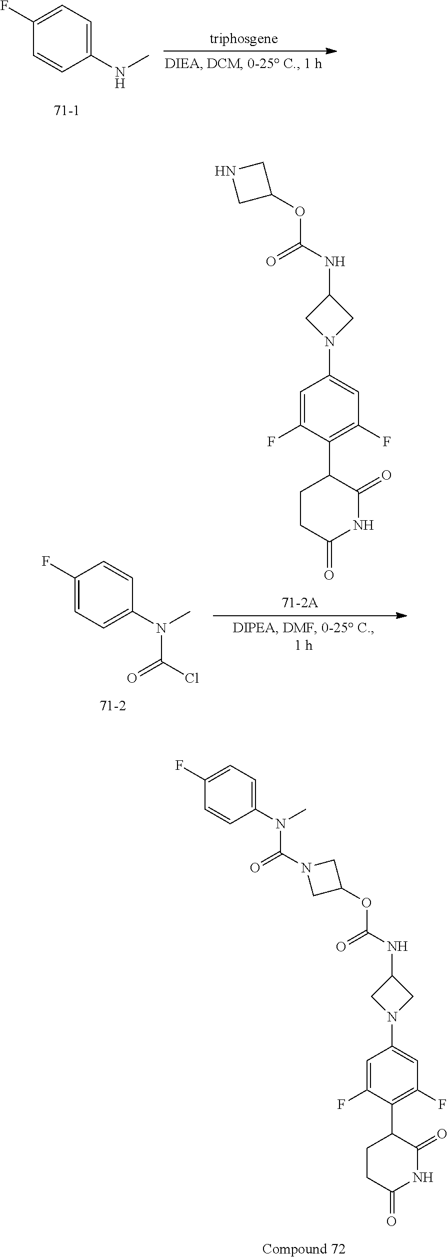

### Step 1. Procedure for Preparation of Compound 2—(4-fluorophenyl)(methyl)carbamic chloride

To a solution of 4-fluoro-N-methylaniline (100 mg, 799 umol, 96.2 uL, 1.00 eq) in dichloromethane (3.00 mL) was added bis(trichloromethyl) carbonate (356 mg, 1.20 mmol, 1.50 eq) and N,N-diisopropylethylamine (310 mg, 2.40 mmol, 418 uL, 3.00 eq) at 0° C. The mixture was stirred at 25° C. for 1 h. The reaction mixture was concentrated in vacuo to afford (4-fluorophenyl)(methyl)carbamic chloride (120 mg, 640 umol, 80% yield) as a brown solid.

### Step 2. Procedure for Preparation of 1-((4-fluorophenyl)(methyl)carbamoyl)azetidin-3-yl (1-(4-(2,6-dioxopiperidin-3-yl)-3,5-difluorophenyl)azetidin-3-yl)carbamate

To a solution of azetidin-3-yl (1-(4-(2,6-dioxopiperidin-3-yl)-3,5-difluorophenyl)azetidin-3-yl)carbamate (79.0 mg, 200 umol, 1.00 eq) in dimethyl formamide (1.00 mL) was added N,N-diisopropylethylamine (77.7 mg, 601 umol, 105 uL, 3 eq) and (4-fluorophenyl)(methyl)carbamic chloride (75.2 mg, 401 umol, 2 eq) at 0° C. The mixture was stirred at 25° C. for 1 h. The reaction was filtered. The filtrate was purified by Prep-HPLC (column: phenomenex luna C18 150*25 mm*10 um; mobile phase: [water(formic acid)-acetonitrile]; B %: 36%-56%, 9 min) and lyophilized to afford 1-((4-fluorophenyl)(methyl)carbamoyl)azetidin-3-yl (1-(4-(2,6-dioxopiperidin-3-yl)-3,5-difluorophenyl)azetidin-3-yl)carbamate (30.65 mg, 50.57 umol, 25% yield, 90% purity) as an off-white solid.

¹H NMR (400 MHz, DMSO-d₆) δ=10.85 (s, 1H), 8.04 (br d, J=7.3 Hz, 1H), 7.35-7.28 (m, 2H), 7.27-7.19 (m, 2H), 6.13 (br d, J=11.0 Hz, 2H), 4.87-4.75 (m, 1H), 4.41-4.30 (m, 1H), 4.10-4.04 (m, 2H), 4.01 (br d, J=4.9 Hz, 1H), 3.81-3.70 (m, 2H), 3.59 (br t, J=6.8 Hz, 2H), 3.36 (br d, J=3.6 Hz, 2H), 3.10 (s, 3H), 2.82-2.72 (m, 1H), 2.58-2.53 (m, 1H), 2.12-2.01 (m, 1H), 1.98-1.89 (m, 1H). MS (ESI) m/z 546.0 [M+H]⁺

### Example 72. Synthesis of Compound 73

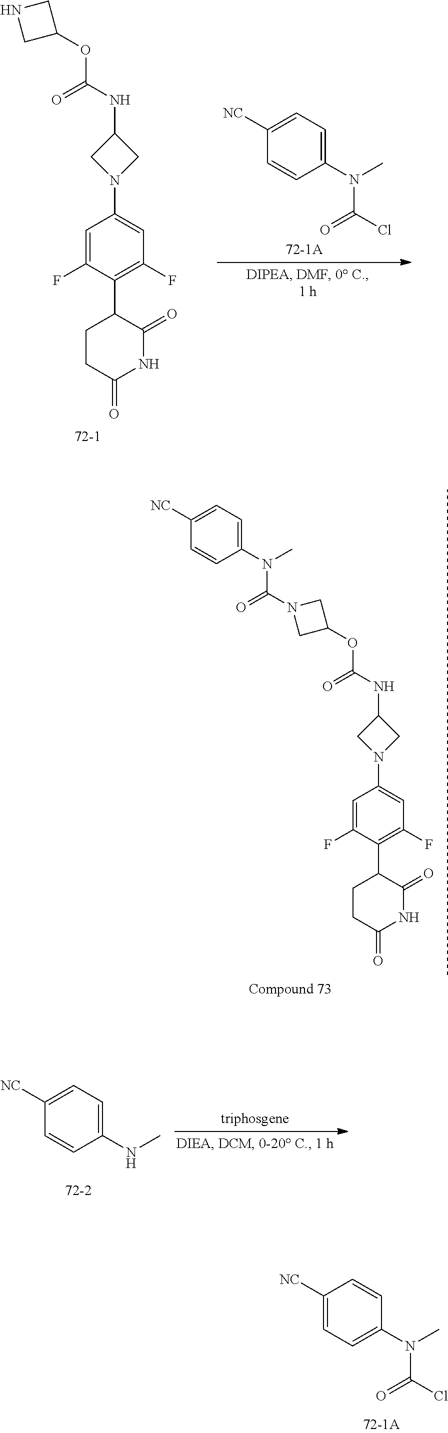

### Step 1. Procedure for Preparation of Compound 1A—(4-cyanophenyl)(methyl)carbamic chloride

To a solution of 4-(methylamino)benzonitrile (60.0 mg, 453 umol, 1.00 eq) in dichloromethane (2.00 mL) was added triphosgene (202 mg, 680 umol, 1.50 eq) and N,N-diisopropylethylamine (176 mg, 1.36 mmol, 237 uL, 3.00 eq) at 0° C. The reaction was stirred at 20° C. for 1 h. The reaction was concentrated under reduced pressure to give a residue. The product (4-cyanophenyl)(methyl)carbamic chloride (80.0 mg, 411 umol, 91% yield) was obtained as a yellow solid.

### Step 2. Procedure for Preparation of 1-((4-cyanophenyl)(methyl)carbamoyl)azetidin-3-yl (1-(4-(2,6-dioxopiperidin-3-yl)-3,5-difluorophenyl)azetidin-3-yl)carbamate

To a solution of azetidin-3-yl (1-(4-(2,6-dioxopiperidin-3-yl)-3,5-difluorophenyl)azetidin-3-yl)carbamate (79.0 mg, 200 umol, 1.00 eq) in dimethylformamide (1.00 mL) was added N,N-diisopropylethylamine (77.6 mg, 600 umol, 104 uL, 3.00 eq) and (4-cyanophenyl)(methyl)carbamic chloride (62.3 mg, 320 umol, 1.60 eq). The reaction was stirred at 0° C. for 1 h. The reaction was filtered to give a filtrate. The filtrate was purified by Prep-HPLC (column: Phenomenex luna C18 150*25 mm*10 um; mobile phase: [water(formic acid)-acetonitrile]; B %: 29%-59%, 9 min) and Prep-HPLC (column: Phenomenex Luna C18 150*30 mm*5 um; mobile phase: [Water-acetonitrile]; B %: 25%-55%, 20 min) and lyophilized to afford 1-((4-cyanophenyl)(methyl)carbamoyl)azetidin-3-yl (1-(4-(2,6-dioxopiperidin-3-yl)-3,5-difluorophenyl)azetidin-3-yl)carbamate (24.77 mg, 43.9 umol, 22% yield, 98% purity) as a white solid.

¹H NMR (400 MHz, DMSO-d₆) δ=10.86 (s, 1H), 8.08 (br d, J=7.2 Hz, 1H), 7.83 (d, J=8.8 Hz, 2H), 7.44 (br d, J=8.4 Hz, 2H), 6.14 (br d, J=11.2 Hz, 2H), 4.90 (br d, J=3.6 Hz, 1H), 4.43-4.33 (m, 1H), 4.10-4.01 (m, 3H), 3.93 (br t, J=8.4 Hz, 2H), 3.61 (br t, J=6.4 Hz, 2H), 3.53 (br dd, J=3.6, 9.6 Hz, 2H), 3.20 (s, 3H), 2.82-2.72 (m, 1H), 2.48-2.45 (m, 1H), 2.10-2.05 (m, 1H), 1.98-1.90 (m, 1H). MS (ESI) m/z 553.3 [M+H]⁺

### Example 73. Synthesis of Compound 74

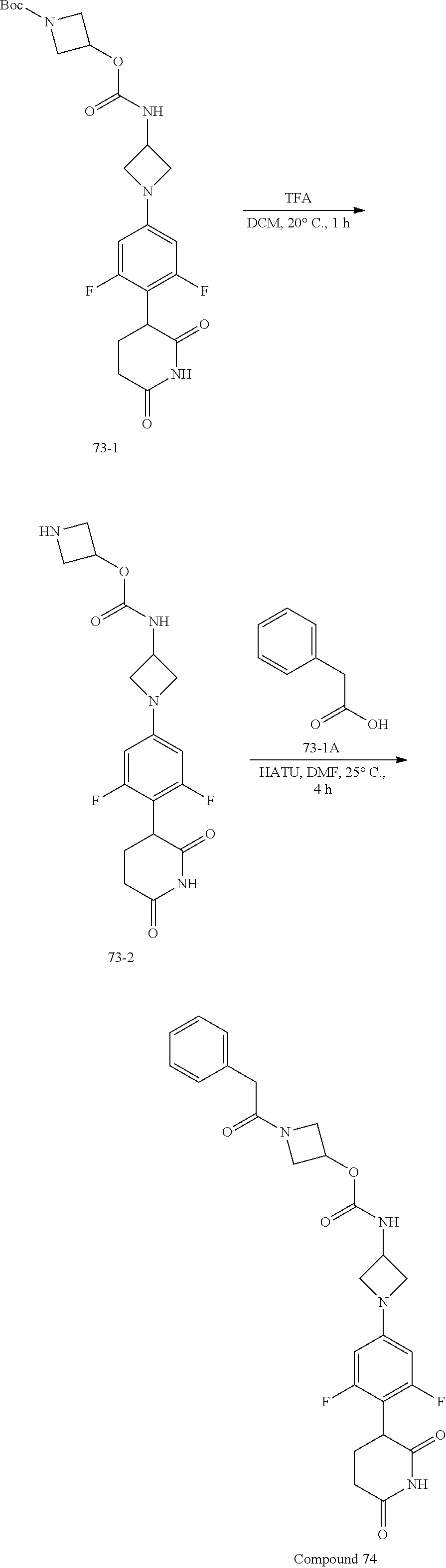 [SMILES: CC(C)(C)OC(=O)N1CC(OC(=O)NC2CN(c3cc(F)c(C4CCC(=O)NC4=O)c(F)c3)C2)C1 ; O=C1CCC(c2c(F)cc(N3CC(NC(=O)OC4CNC4)C3)cc2F)C(=O)N1 ; O=C(O)Cc1ccccc1 ; "TFA" ; O=C1CCC(c2c(F)cc(N3CC(NC(=O)OC4CN(C(=O)Cc5ccccc5)C4)C3)cc2F)C(=O)N1]

### Step 1. Procedure for Preparation of Compound 2—azetidin-3-yl (1-(4-(2,6-dioxopiperidin-3-yl)-3,5-difluorophenyl)azetidin-3-yl)carbamate

To a solution of tert-butyl 3-(((1-(4-(2,6-dioxopiperidin-3-yl)-3,5-difluorophenyl)azetidin-3-yl)carbamoyl)oxy)azetidine-1-carboxylate (100 mg, 202 umol, 1.00 eq) in dichloromethane (2.00 mL) were added trifluoroacetic acid (616 mg, 5.40 mmol, 0.400 mL, 26.7 eq). The reaction mixture was stirred at 20° C. for 1 h. The reaction mixture was concentrated under reduced pressure to afford azetidin-3-yl (1-(4-(2,6-dioxopiperidin-3-yl)-3,5-difluorophenyl)azetidin-3-yl)carbamate (102 mg, 201 umol, 99% yield, trifluoroformate) as yellow oil, and directly used to next step. MS (ESI) m/z 394.9 [M+H]⁺

### Step 2. Procedure for Preparation of 1-(2-phenylacetyl)azetidin-3-yl (1-(4-(2,6-dioxopiperidin-3-yl)-3,5-difluorophenyl)azetidin-3-yl)carbamate

To a solution of 2-phenylacetic acid (17.3 mg, 127 umol, 16.0 uL, 1.00 eq) in dimethylformamide (2 mL) were added N,N-diisopropylethylamine (49.2 mg, 380 umol, 66.3 uL, 3.00 eq), azetidin-3-yl (1-(4-(2,6-dioxopiperidin-3-yl)-3,5-difluorophenyl)azetidin-3-yl)carbamate (100 mg, 254 umol, 2.00 eq) and 2-(3H-[1,2,3]triazolo[4,5-b]pyridin-3-yl)-1,1,3,3-tetramethyluronium hexafluorophosphate(V) (72.3 mg, 190 umol, 1.50 eq). The reaction mixture was stirred at 25° C. for 4 h. The reaction mixture was diluted with water (2 mL) and extracted with ethyl acetate (2×3 mL). The combined layers was concentrated under reduced pressure to give a residue. The residue was purified by Prep-HPLC (column: Phenomenex C18 150*25 mm*10 um; mobile phase: [water (ammonium bicarbonate)-acetonitrile]; B %: 20%-50%, 8 min) and lyophilized to give a white solid. The white solid was purified by Prep-TLC (UV 254 nm, petroleum ether:ethyl acetate=0:1, R_{f}=0.59) to afford a crude product. The crude product was triturated with petroleum ether (10 ml) at 25° C. for 1 h to afford [1-(2-phenylacetyl)azetidin-3-yl]N-[1-[4-(2,6-dioxo-3-piperidyl)-3,5-difluoro-phenyl]azetidin-3-yl]carbamate (17.24 mg, 33.3 umol, 26.27% yield, 99% purity) as a white solid.

¹H NMR (400 MHz, DMSO-d₆) δ=10.85 (s, 1H), 8.13 (br d, J=7.3 Hz, 1H), 7.33-7.26 (m, 2H), 7.25-7.17 (m, 3H), 6.14 (d, J=10.9 Hz, 2H), 5.10-5.01 (m, 1H), 4.49-4.38 (m, 2H), 4.16-4.00 (m, 5H), 3.71 (br dd, J=3.9, 10.8 Hz, 1H), 3.63 (br t, J=6.8 Hz, 2H), 3.43 (s, 2H), 2.83-2.71 (m, 1H), 2.60-2.54 (m, 1H), 2.11-2.02 (m, 1H), 1.97-1.88 (m, 1H). MS (ESI) m/z. 512.9 [M+H]⁺

### Example 74. Synthesis of Compound 75

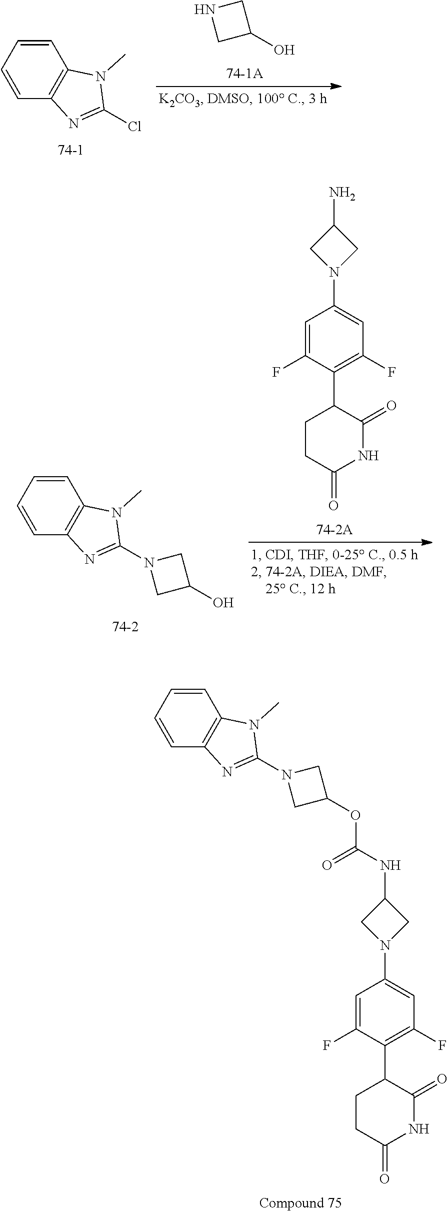 [SMILES: Cn1c(Cl)nc2ccccc21 ; OC1CNC1 ; Cn1c(N2CC(O)C2)nc2ccccc21 ; NC1CN(c2cc(F)c(C3CCC(=O)NC3=O)c(F)c2)C1 ; Cn1c(N2CC(OC(=O)NC3CN(c4cc(F)c(C5CCC(=O)NC5=O)c(F)c4)C3)C2)nc2ccccc21]

### Step 1. Procedure for Preparation of Compound 2—1-(1-methyl-1H-benzo[d]imidazol-2-yl)azetidin-3-ol

To a solution of azetidin-3-ol (236 mg, 2.16 mmol, 1.20 eq, hydrochloric acid) and 2-chloro-1-methyl-1H-benzo[d]imidazole (300 mg, 1.80 mmol, 1.00 eq) in dimethylsulfoxide (2.00 mL) was added potassium carbonate (746 mg, 5.40 mmol, 3.00 eq). The reaction was stirred at 100° C. for 3 h. The reaction was filtered to give a filtrate. The filtrate was purified by reversed-phase HPLC (0.1% formic acid condition) to afford 1-(1-methyl-1H-benzo[d]imidazol-2-yl)azetidin-3-ol (150 mg, 738 umol, 41% yield) as a white solid.

¹H NMR (400 MHz, DMSO-d₆) δ=7.33-7.20 (m, 2H), 7.08-6.96 (m, 2H), 5.72 (s, 1H), 4.59 (br t, J=5.6 Hz, 1H), 4.39-4.30 (m, 2H), 3.97-3.89 (m, 2H), 3.52 (s, 3H). MS (ESI) m/z 203.8 [M+H]⁺

### Step 2. Procedure for Preparation of 1-(1-methyl-1H-benzo[d]imidazol-2-yl)azetidin-3-yl (1-(4-(2,6-dioxopiperidin-3-yl)-3,5-difluorophenyl)azetidin-3-yl)carbamate

To a solution of 1-(1-methyl-1H-benzo[d]imidazol-2-yl)azetidin-3-ol (40.0 mg, 196 umol, 1.00 eq) in tetrahydrofuran (1.00 mL) was added di(1H-imidazol-1-yl)methanone (31.9 mg, 196 umol, 1.00 eq) at 0° C. The reaction was stirred at 25° C. for 0.5 h to give a resulting solution. A solution of 3-(4-(3-aminoazetidin-1-yl)-2,6-difluorophenyl)piperidine-2,6-dione (54.6 mg, 184 umol, 1.00 eq), 2,3,4,6,7,8,9,10-octahydropyrimido[1,2-a]azepine (28.1 mg, 184 umol, 27.8 uL, 1.00 eq), N,N-diisopropylethylamine (23.9 mg, 184 umol, 32.2 uL, 1.00 eq) in dimethylformamide (1.00 mL) was added to the resulting solution at 25° C. The reaction was stirred at 25° C. for 12 h. The reaction was concentrated under reduced pressure and filtered to give residue. The residue was purified by Prep-HPLC (column: Phenomenex luna C18 150*25 mm*10 um; mobile phase: [water(formic acid)-acetonitrile]; B %: 9%-39%, 9 min) to afford 1-(1-methyl-1H-benzo[d]imidazol-2-yl)azetidin-3-yl (1-(4-(2,6-dioxopiperidin-3-yl)-3,5-difluorophenyl)azetidin-3-yl)carbamate (33.51 mg, 62.61 umol, 34% yield, 98% purity) as a white solid.

¹H NMR (400 MHz, DMSO-d₆) δ=10.86 (s, 1H), 8.18-8.14 (m, 1H), 7.37-7.23 (m, 2H), 7.08-6.95 (m, 2H), 6.16 (br d, J=11.2 Hz, 2H), 5.22 (br t, J=4.4 Hz, 1H), 4.49 (br dd, J=7.2, 8.3 Hz, 2H), 4.47-4.37 (m, 1H), 4.17-4.08 (m, 4H), 4.04 (br dd, J=4.8, 12.7 Hz, 1H), 3.67 (br t, J=6.8 Hz, 2H), 3.53 (s, 3H), 2.84-2.73 (m, 1H), 2.50-2.43 (m, 1H), 2.13-2.01 (m, 1H), 2.00-1.91 (m, 1H). MS (ESI) m/z 525.4 [M+H]⁺

### Example 75. Synthesis of Compound 76

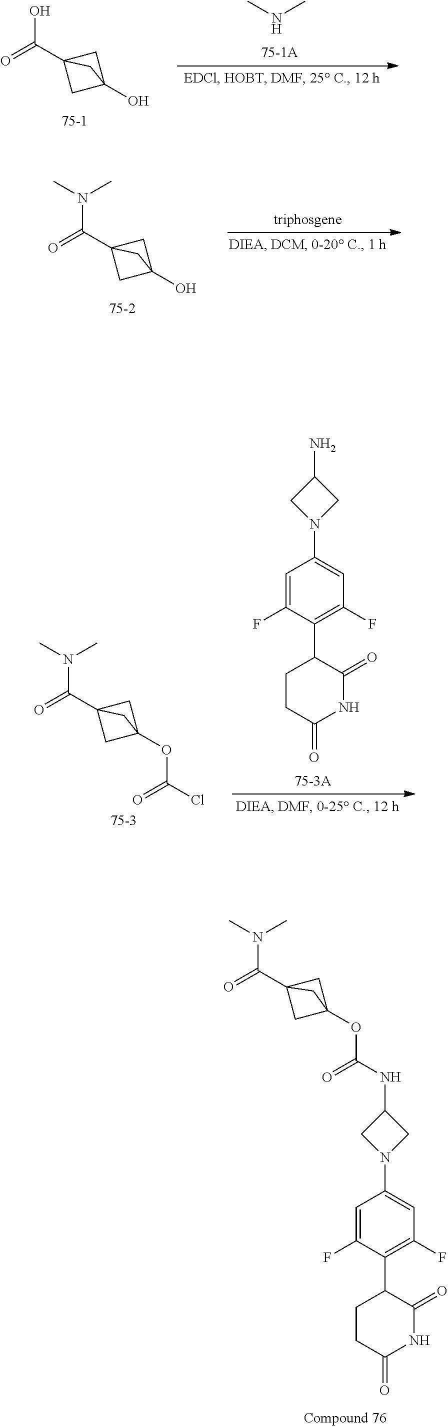

### Step 1. Procedure for Preparation of Compound 2—3-hydroxy-N,N-dimethylbicyclo[1.1.1]pentane-1-carboxamide

To a solution of 3-hydroxybicyclo[1.1.1]pentane-1-carboxylic acid (50.0 mg, 390 umol, 1.00 eq) in dimethyl formamide (1.00 mL) was added 1-hydroxybenzotriazole (36.9 mg, 273 umol, 0.70 eq), 1-(3-dimethylaminopropyl)-3-ethylcarbodiimide hydrochloride (82.3 mg, 429 umol, 1.10 eq) and N,N-diisopropylethylamine (101 mg, 780 umol, 136 uL, 2.00 eq) followed by dimethylamine (318 mg, 3.90 mmol, 10.0 eq, hydrochloride). The mixture was stirred at 25° C. for 12 h. The reaction was filtered. The filtrate was purified by Prep-HPLC (column: Phenomenex luna C18 150*25 mm*10 um; mobile phase: [water (formic acid)-acetonitrile]; B %: 0%-20%, 10 min) and lyophilized to afford 3-hydroxy-N,N-dimethylbicyclo[1.1.1]pentane-1-carboxamide (50.0 mg, 258 umol, 66% yield, 80% purity) as a white solid.

¹H NMR (400 MHz, DMSO-d₆) δ=6.32 (s, 1H), 2.99 (s, 3H), 2.78 (s, 3H), 2.09-2.01 (m, 6H).

### Step 2. Procedure for Preparation of Compound 3—3-(dimethylcarbamoyl)bicyclo[1.1.1]pentan-1-yl carbonochloridate

To a solution of 3-hydroxy-N,N-dimethylbicyclo[1.1.1]pentane-1-carboxamide (70.0 mg, 451 umol, 1.00 eq) in dichloromethane (3.00 mL) was added bis(trichloromethyl) carbonate (201 mg, 677 umol, 1.50 eq) and N,N-diisopropylethylamine (87.4 mg, 677 umol, 118 uL, 1.50 eq) at 0° C. The mixture was stirred at 20° C. for 1 h. The reaction mixture was concentrated under reduced pressure to afford 3-(dimethylcarbamoyl) bicyclo[1.1.1]pentan-1-yl carbonochloridate (90.0 mg, 414 umol, 92% yield) as white oil.

### Step 3. Procedure for Preparation of 3-(dimethylcarbamoyl)bicyclo[1.1.1]pentan-1-yl (1-(4-(2,6-dioxopiperidin-3-yl)-3,5-difluorophenyl)azetidin-3-yl)carbamate

To a solution of 3-(4-(3-aminoazetidin-1-yl)-2,6-difluorophenyl)piperidine-2,6-dione (122 mg, 414 umol, 1.00 eq, mesylate) in dimethyl formamide (2.00 mL) was added N,N-diisopropylethylamine (160 mg, 1.24 mmol, 216 uL, 3.00 eq) followed by 3-(dimethylcarbamoyl)bicyclo[1.1.1]pentan-1-yl carbonochloridate (90.0 mg, 414 umol, 1.00 eq) at 0° C. The mixture was stirred at 25° C. for 12 h. The reaction was filtered. The filtrate was purified by Prep-HPLC (column: Phenomenex luna C18 150*25 mm*10 um; mobile phase: [water(formic acid)-acetonitrile]; B %: 23%-53%, 10 min) and lyophilized to afford 3-(dimethylcarbamoyl)bicyclo[1.1.1]pentan-1-yl (1-(4-(2,6-dioxopiperidin-3-yl)-3,5-difluorophenyl)azetidin-3-yl)carbamate (12.63 mg, 12.47 umol, 4% yield, 95% purity, formate) as a white solid.

¹H NMR (400 MHz, DMSO-d₆) δ=10.86 (s, 1H), 8.50-8.44 (m, 1H), 7.97 (br d, J=7.5 Hz, 1H), 6.15 (d, J=11.1 Hz, 2H), 4.48-4.34 (m, 1H), 4.08 (br t, J=7.5 Hz, 2H), 4.03 (br dd, J=5.4, 12.8 Hz, 1H), 3.62 (br t, J=6.6 Hz, 2H), 3.01 (s, 3H), 2.81 (s, 3H), 2.79-2.72 (m, 1H), 2.48-2.46 (m, 1H), 2.36 (s, 6H), 2.13-2.01 (m, 1H), 1.99-1.89 (m, 1H). MS (ESI) m/z 477.1 [M+H]⁺

### Example 76. Synthesis of Compound 77

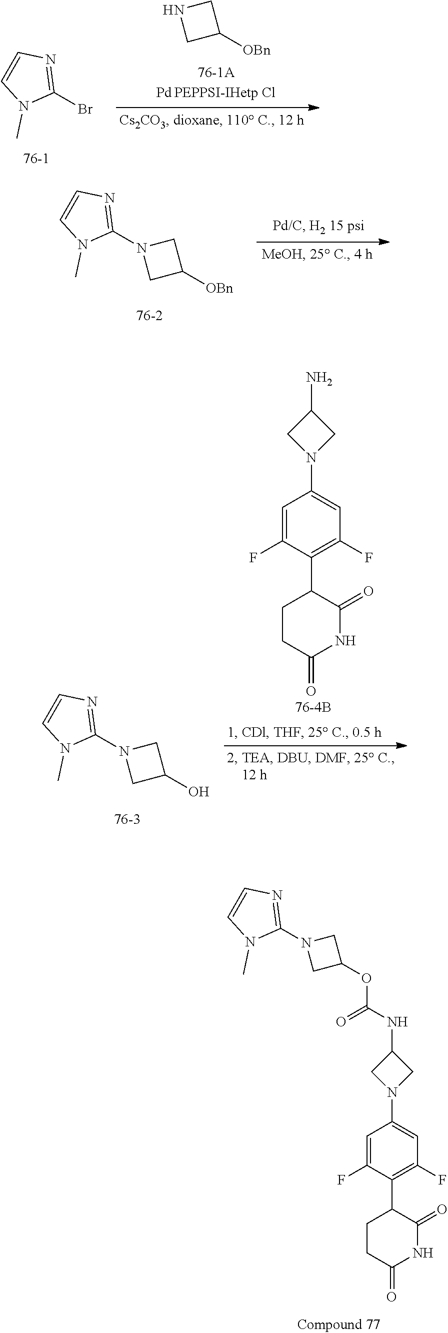

### Step 1. Procedure for Preparation of Compound 2—2-(3-(benzyloxy)azetidin-1-yl)-1-methyl-1H-imidazole

A mixture of 2-bromo-1-methyl-1H-imidazole (200 mg, 1.24 mmol, 1.00 eq), 3-(benzyloxy)azetidine (496 mg, 2.48 mmol, 2.00 eq, hydrochloride), 1,3-bis[2,6-bis(1-propylbutyl)phenyl]-4,5-dichloro-2H-imidazol-1-ium-2-ide;3-chloropyridine;dichloropalladium (121 mg, 124 umol, 0.100 eq), cesium carbonate (1.21 g, 3.73 mmol, 3.00 eq) in dioxane (2.00 mL) was degassed and purged with nitrogen atmosphere for 3 times. The mixture was stirred at 100° C. for 12 h under nitrogen atmosphere. The reaction mixture was filtered and concentrated under reduced pressure to give a residue. The residue was purified by reverse phase chromatography (C18, 40 g; condition: water/acetonitrile=100:0 to 0:100, 0.1% formic) and lyophilized to afford 2-(3-(benzyloxy)azetidin-1-yl)-1-methyl-1H-imidazole (300 mg, 1.23 mmol, 99% yield) as yellow oil.

¹H NMR (400 MHz, DMSO-d₆) δ=7.41-7.31 (m, 5H), 6.73 (s, 1H), 6.48 (s, 1H), 4.46 (s, 2H), 4.44-4.39 (m, 1H), 4.06 (d, J=6.8 Hz, 2H), 3.80-3.76 (m, 2H), 3.33 (s, 3H)

### Step 2. Procedure for Preparation of Compound 3—1-(1-methyl-1H-imidazol-2-yl)azetidin-3-ol

To a solution of 2-(3-(benzyloxy)azetidin-1-yl)-1-methyl-1H-imidazole (200 mg, 822 umol, 1.00 eq) in methanol (2.00 mL) was added palladium/carbon (100 mg, 822 umol, 10% purity, 1.00 eq) at 25 C. The mixture was degassed and purged with hydrogen atmosphere for 3 times. The mixture was stirred at 25° C. for 4 h under hydrogen atmosphere. The reaction mixture was filtered and concentrated under reduced pressure to afford 1-(1-methyl-1H-imidazol-2-yl)azetidin-3-ol (72.0 mg, 470 umol, 57% yield) as colorless oil.

¹H NMR (400 MHz, DMSO-d₆) δ=6.71 (s, 1H), 6.47 (d, J=1.1 Hz, 1H), 4.52-4.42 (m, 1H), 4.04 (dd, J=6.8, 8.0 Hz, 2H), 3.74-3.65 (m, 2H), 3.39-3.35 (m, 3H). MS (ESI) m/z 154.2 [M+H]⁺

### Step 3. Procedure for Preparation of 1-(1-methyl-1H-imidazol-2-yl)azetidin-3-yl (1-(4-(2,6-dioxopiperidin-3-yl)-3,5-difluorophenyl)azetidin-3-yl)carbamate

To a solution of 1-(1-methyl-1H-imidazol-2-yl)azetidin-3-ol (72.0 mg, 470.03 umol, 1.00 eq) in tetrahydrofuran (5.00 mL) was added 1,1′-carbonyldiimidazole (83.8 mg, 517 umol, 1.10 eq). The mixture was stirred at 25° C. for 0.5 h. The reaction mixture was concentrated under reduced pressure to afford 2-(3-((1H-imidazol-1-yl)oxy)azetidin-1-yl)-1-methyl-1H-imidazole (116 mg, 469 umol, 99% yield) as colorless oil. To a solution of 3-[4-(3-aminoazetidin-1-yl)-2,6-difluoro-phenyl]piperidine-2,6-dione (180 mg, 460 umol, 1.00 eq, methanesulfonic acid) in N,N-dimethyl formamide (5.00 mL) was added triethylamine (46.5 mg, 460 umol, 64.0 uL, 1.00 eq) and 1,8-diazabicyclo[5.4.0]undec-7-ene (70.0 mg, 460 umol, 69.3 uL, 1.00 eq) and 2-(3-((1H-imidazol-1-yl)oxy)azetidin-1-yl)-1-methyl-1H-imidazole (114 mg, 460 umol, 1.00 eq) at 25° C. The mixture was stirred at 25° C. for 12 h. The reaction mixture was concentrated under reduced pressure to give a residue. The residue was purified by prep-HPLC (column: Phenomenex Luna C18 150*25 mm*10 um; mobile phase: [water(formic)-acetonitrile]; B %: 3%-33%, 10 min) and lyophilized to afford 1-(1-methyl-1H-imidazol-2-yl)azetidin-3-yl (1-(4-(2,6-dioxopiperidin-3-yl)-3,5-difluorophenyl)azetidin-3-yl)carbamate (23.39 mg, 48.81 umol, 10.61% yield, 99% purity) as a white solid.

¹H NMR (400 MHz, DMSO-d₆) δ=10.86 (s, 1H), 8.12 (br d, J=6.6 Hz, 1H), 6.75 (s, 1H), 6.50 (s, 1H), 6.16 (br d, J=11.1 Hz, 2H), 5.19-4.99 (m, 1H), 4.51-4.36 (m, 1H), 4.25-4.14 (m, 2H), 4.11 (br t, J=7.8 Hz, 2H), 4.05-3.98 (m, 1H), 3.89-3.78 (m, 2H), 3.70-3.60 (m, 2H), 3.35-3.34 (m, 3H), 2.82-2.72 (m, 1H), 2.57-2.54 (m, 1H), 2.13-2.05 (m, 1H), 2.00-1.92 (m, 1H)

¹H NMR (400 MHz, DMSO-d₆) δ=6.74 (s, 1H), 6.48 (s, 1H), 6.14 (d, J=11.3 Hz, 2H), 5.18-5.05 (m, 1H), 4.42 (br dd, J=6.0, 6.8 Hz, 1H), 4.20 (t, J=7.7 Hz, 2H), 4.10 (br t, J=7.4 Hz, 2H), 4.02 (br dd, J=4.8, 12.3 Hz, 1H), 3.83 (br dd, J=4.5, 8.6 Hz, 2H), 3.66 (br s, 2H), 3.33 (s, 3H), 2.81-2.72 (m, 1H), 2.59-2.55 (m, 1H), 2.14-1.99 (m, 1H), 1.99-1.87 (m, 1H). MS (ESI) m/z 475.2 [M+H]⁺

### Example 77. Synthesis of Compound 78

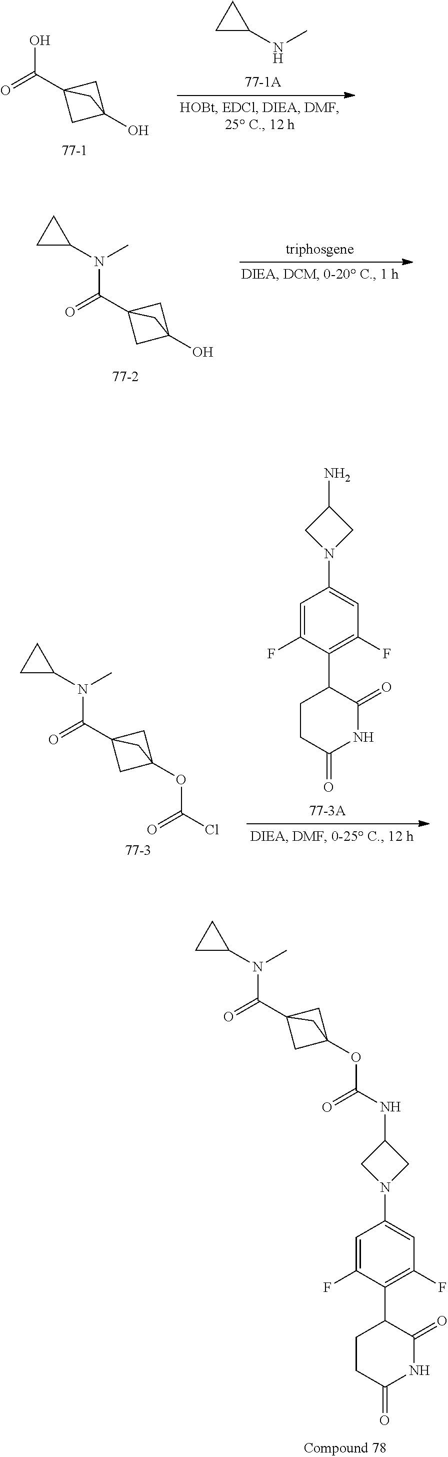

### Step 1. Procedure for Preparation of Compound 2—N-cyclopropyl-3-hydroxy-N-methylbicyclo[1.1.1]pentane-1-carboxamide

To a solution of 3-hydroxybicyclo[1.1.1]pentane-1-carboxylic acid (100 mg, 780 umol, 1.00 eq) in dimethyl formamide (5.00 mL) was added 1-hydroxybenzotriazole (73.8 mg, 546 umol, 0.700 eq), 1-(3-dimethylaminopropyl)-3-ethylcarbodiimide hydrochloride (64.6 mg, 859 umol, 1.10 eq) and N,N-diisopropylethylamine (202 mg, 1.56 mmol, 272 uL, 2.00 eq) followed by N-methylcyclopropanamine (555 mg, 7.80 mmol, 10.0 eq). The mixture was stirred at 25° C. for 12 h. The reaction was filtered. The filtrate was purified by Prep-HPLC (column: Phenomenex luna C18 150*25 mm*10 um; mobile phase: [water (formic acid)-acetonitrile]; B %: 2%-32%, 9 min) and lyophilized to afford N-cyclopropyl-3-hydroxy-N-methylbicyclo[1.1.1]pentane-1-carboxamide (70.0 mg, 344 umol, 44% yield, 89% purity) as a white solid.

¹H NMR (400 MHz, DMSO-d₆) δ=6.28 (s, 1H), 2.97-2.80 (m, 1H), 2.75 (br s, 3H), 2.09 (br s, 6H), 0.87-0.52 (m, 4H).

### Step 2. Procedure for Preparation of Compound 3—3-(cyclopropyl(methyl)carbamoyl)bicyclo[1.1.1]pentan-1-yl carbonochloridate

To a solution of N-cyclopropyl-3-hydroxy-N-methylbicyclo[1.1.1]pentane-1-carboxamide (50.0 mg, 276 umol, 1.00 eq) in dichloromethane (3.00 mL) was added bis(trichloromethyl) carbonate (123 mg, 414 umol, 1.50 eq) and N,N-diisopropylethylamine (53.5 mg, 414 umol, 72.1 uL, 1.50 eq) at 0° C. The mixture was stirred at 20° C. for 1 h. The reaction mixture was concentrated in vacuo to afford 3-(cyclopropyl(methyl)carbamoyl)bicyclo[1.1.1]pentan-1-yl carbonochloridate (60.0 mg, 246 umol, 89% yield) as white oil.

### Step 3. Procedure for Preparation of 3-(cyclopropyl(methyl)carbamoyl)bicyclo[1.1.1]pentan-1-yl (1-(4-(2,6-dioxopiperidin-3-yl)-3,5-difluorophenyl)azetidin-3-yl)carbamate

To a solution of 3-(4-(3-aminoazetidin-1-yl)-2,6-difluorophenyl)piperidine-2,6-dione (72.7 mg, 246 umol, 1.00 eq, mesylate) in dimethyl formamide (2.00 mL) was added N,N-diisopropylethylamine (95.5 mg, 739 umol, 129 uL, 3.00 eq) followed by 3-(cyclopropyl(methyl)carbamoyl)bicyclo[1.1.1]pentan-1-yl carbonochloridate (60.0 mg, 246 umol, 1.00 eq) at 0° C. The mixture was stirred at 25° C. for 12 h. The reaction was filtered. The filtrate was purified by Prep-HPLC (column: Welch Ultimate C18 150*25 mm*5 um; mobile phase: [water (formic acid)-acetonitrile]; B %: 28%-58%, 10 min) and lyophilized to afford 3-(cyclopropyl(methyl)carbamoyl)bicyclo[1.1.1]pentan-1-yl(1-(4-(2,6-dioxopiperidin-3-yl)-3,5-difluorophenyl) azetidin-3-yl)carbamate (12.55 mg, 25.0 umol, 10% yield, 95% purity) as a white solid.

¹H NMR (400 MHz, DMSO-d₆) δ=10.86 (s, 1H), 7.96 (br d, J=7.5 Hz, 1H), 6.15 (br d, J=11.1 Hz, 2H), 4.51-4.28 (m, 1H), 4.08 (br t, J=7.8 Hz, 2H), 4.03 (br dd, J=5.3, 12.8 Hz, 1H), 3.62 (br t, J=6.7 Hz, 2H), 2.92-2.70 (m, 5H), 2.48-2.45 (m, 1H), 2.40 (br s, 6H), 2.13-2.01 (m, 1H), 2.00-1.89 (m, 1H), 0.90-0.53 (m, 4H). MS (ESI) m/z 503.1 [M+H]⁺

### Example 78. Synthesis of Compound 79

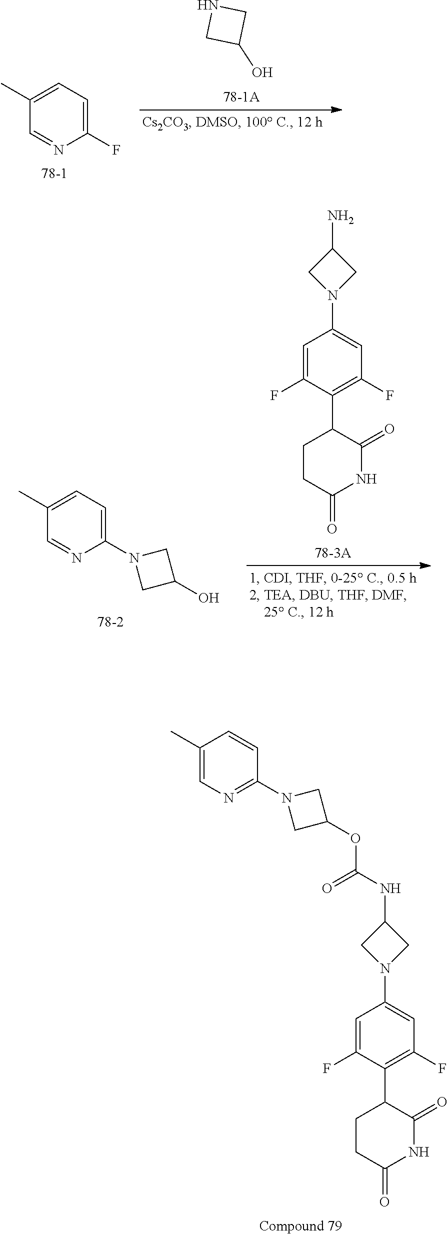

### Step 1. Procedure for Compound 2—1-(5-methylpyridin-2-yl)azetidin-3-ol

To a solution of 2-fluoro-5-methyl-pyridine (500 mg, 4.50 mmol, 467 uL, 1.00 eq) in dimethylsulfoxide (8.00 mL) was added cesium carbonate (2.93 g, 9.00 mmol, 2.00 eq) and azetidin-3-ol (788 mg, 7.20 mmol, 1.60 eq, hydrochloride). The mixture was stirred at 100° C. for 12 h. The reaction mixture was concentrated under reduced pressure to give a residue. The residue was purified by reverse phase chromatography (C18, 40 g; condition: water/acetonitrile=100:0 to 0:100, 0.1% formic acid) and lyophilized to afford 1-(5-methyl-2-pyridyl)azetidin-3-ol (590 mg, 3.56 mmol, 79% yield, 99% purity) as yellow oil.

¹H NMR (400 MHz, DMSO-d₆) δ=7.84 (dd, J=0.7, 1.6 Hz, 1H), 7.29 (dd, J=2.3, 8.4 Hz, 1H), 6.27 (d, J=8.4 Hz, 1H), 5.67-5.42 (m, 1H), 4.49 (br d, J=4.6 Hz, 1H), 4.09-3.99 (m, 2H), 3.54 (dd, J=4.9, 8.8 Hz, 2H), 2.08 (s, 3H)

### Step 2. Procedure for 1-(5-methylpyridin-2-yl)azetidin-3-yl (1-(4-(2,6-dioxopiperidin-3-yl)-3,5-difluorophenyl)azetidin-3-yl)carbamate

To a solution of 1-(5-methylpyridin-2-yl)azetidin-3-ol (200 mg, 1.22 mmol, 1.00 eq) in tetrahydrofuran (2.00 mL) were added 1,1′-carbonyldiimidazole (217 mg, 1.34 mmol, 1.10 eq) at 0° C. The mixture was stirred at 25° C. for 0.5 h. The reaction mixture was concentrated under reduced pressure to afford 1-(5-methylpyridin-2-yl)azetidin-3-yl 1H-imidazole-1-carboxylate (314 mg, crude) as colorless oil. To a solution of 3-(4-(3-aminoazetidin-1-yl)-2,6-difluorophenyl)piperidine-2,6-dione (470 mg, 1.20 mmol, 1.00 eq, methanesulfonic acid) in tetrahydrofuran (2.00 mL) and N,N-dimethyl formamide (4.00 mL) were added triethylamine (121 mg, 1.20 mmol, 167 uL, 1.00 eq), 1,8-diazabicyclo[5.4.0]undec-7-ene (183 mg, 1.20 mmol, 181 uL, 1.00 eq), and 1-(5-methylpyridin-2-yl)azetidin-3-yl 1H-imidazole-1-carboxylate (310 mg, 1.20 mmol, 1.00 eq). The mixture was stirred at 25° C. for 12 h. The reaction mixture was concentrated under reduced pressure to give a residue. The residue was purified by prep-HPLC (column: Waters xbridge 150*25 mm 10 um; mobile phase: [water(ammonium bicarbonate)-acetonitrile]; B %: 25%-55%, 8 min) and lyophilized to afford 1-(5-methylpyridin-2-yl)azetidin-3-yl (1-(4-(2,6-dioxopiperidin-3-yl)-3,5-difluorophenyl)azetidin-3-yl)carbamate (43.07 mg, 79.9 umol, 6.66% yield, 97% purity) as a white solid.

¹H NMR (400 MHz, DMSO-d₆) δ=10.85 (s, 1H), 8.17-8.06 (m, 1H), 7.91 (s, 1H), 7.39-7.34 (m, 1H), 6.37 (d, J=8.4 Hz, 1H), 6.15 (br d, J=11.1 Hz, 2H), 5.21-5.13 (m, 1H), 4.48-4.38 (m, 1H), 4.23-4.15 (m, 2H), 4.10 (br t, J=7.6 Hz, 2H), 4.03 (br dd, J=4.9, 12.2 Hz, 1H), 3.76 (br dd, J=3.9, 9.2 Hz, 2H), 3.65 (br t, J=6.6 Hz, 2H), 2.83-2.71 (m, 1H), 2.58-2.52 (m, 1H), 2.14 (s, 3H), 2.10-2.03 (m, 1H), 1.99-1.91 (m, 1H). MS (ESI) m/z 486.5 [M+H]⁺

### Example 79. Synthesis of Compound 80

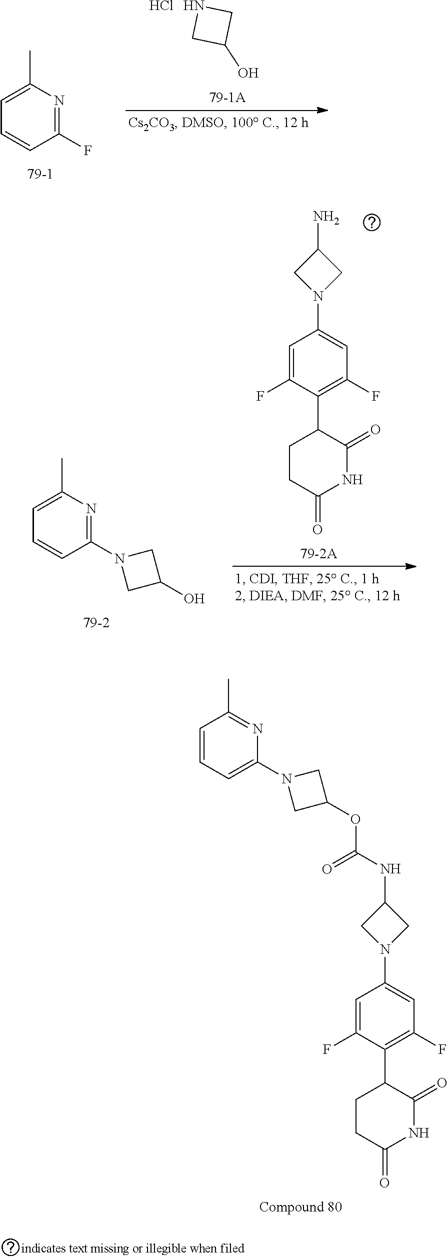

### Step 1. Procedure for Preparation of Compound 2—1-(6-methylpyridin-2-yl)azetidin-3-ol

To a solution of 2-fluoro-6-methyl-pyridine (1.00 g, 9.00 mmol, 925 uL, 1.00 eq) in dimethylsulfoxide (20.0 mL) were added cesium carbonate (8.80 g, 27.0 mmol, 3.00 eq) and azetidin-3-ol (1.97 g, 18.0 mmol, 2.00 eq, hydrochloride). The mixture was stirred at 100° C. for 12 h. The reaction mixture was concentrated under reduce pressure to give a residue. The residue was purified by column chromatography (SiO₂, petroleum ether/ethyl acetate=3/1 to 1/1) to afford 1-(6-methylpyridin-2-yl)azetidin-3-ol (750 mg, 4.57 mmol, 50% yield) as a white solid

¹H NMR (400 MHz, DMSO-d₆) δ=7.36 (t, J=7.8 Hz, 1H), 6.46 (d, J=7.3 Hz, 1H), 6.14 (d, J=8.3 Hz, 1H), 5.58 (d, J=6.6 Hz, 1H), 4.61-4.45 (m, 1H), 4.14-4.08 (m, 2H), 3.61 (dd, J=4.8, 8.7 Hz, 2H), 2.27 (s, 3H)

### Step 2. Procedure for Preparation of 1-(6-methylpyridin-2-yl)azetidin-3-yl (1-(4-(2,6-dioxopiperidin-3-yl)-3,5-difluorophenyl)azetidin-3-yl)carbamate

To a solution of 1-(6-methylpyridin-2-yl)azetidin-3-ol (50.0 mg, 304 umol, 1.00 eq) in tetrahydrofuran (1.00 mL) were added di(1H-imidazol-1-yl)methanone (59.3 mg, 365 umol, 1.20 eq). The mixture was stirred at 25° C. for 1 h. The resulting solution was added to a solution of 3-(4-(3-aminoazetidin-1-yl)-2,6-difluorophenyl)piperidine-2,6-dione (118 mg, 302 umol, 1.00 eq, mesylate), N,N-diisopropylethylamine (58.5 mg, 453 umol, 78.9 uL, 1.50 eq) in tetrahydrofuran (1.00 mL) and N,N-dimethylformamide (1.00 mL). The reaction mixture was stirred at 25° C. for 12 h. The reaction mixture was concentrated under reduced pressure to give a residue. The residue was purified by reverse phase chromatography (C18, 40 g; condition: water/acetonitrile=100:0 to 0:100, 0.1% formic acid) and lyophilized to afford 1-(6-methylpyridin-2-yl)azetidin-3-yl (1-(4-(2,6-dioxopiperidin-3-yl)-3,5-difluorophenyl)azetidin-3-yl)carbamate (13.4 mg, 23.95 umol, 7 yield, 95% purity, formic acid) as an off-white solid.

¹H NMR (400 MHz, DMSO-d₆) δ=10.93-10.81 (m, 1H), 8.28-8.25 (m, 1H), 8.11 (br d, J=7.1 Hz, 1H), 7.47 (s, 1H), 6.59-6.48 (m, 1H), 6.21 (br d, J=7.9 Hz, 1H), 6.15 (br d, J=11.0 Hz, 2H), 5.18 (br d, J=4.1 Hz, 1H), 4.51-4.37 (m, 1H), 4.22 (br dd, J=6.6, 8.9 Hz, 2H), 4.11 (br t, J=7.3 Hz, 2H), 4.07-4.02 (m, 1H), 3.82-3.74 (m, 2H), 3.71-3.62 (m, 2H), 2.85-2.72 (m, 1H), 2.29 (s, 3H), 2.15-2.01 (m, 2H), 1.94 (dt, J=2.1, 6.2 Hz, 1H). MS (ESI) m/z. 486.3 [M+H]⁺

### Example 80. Synthesis of Compound 81

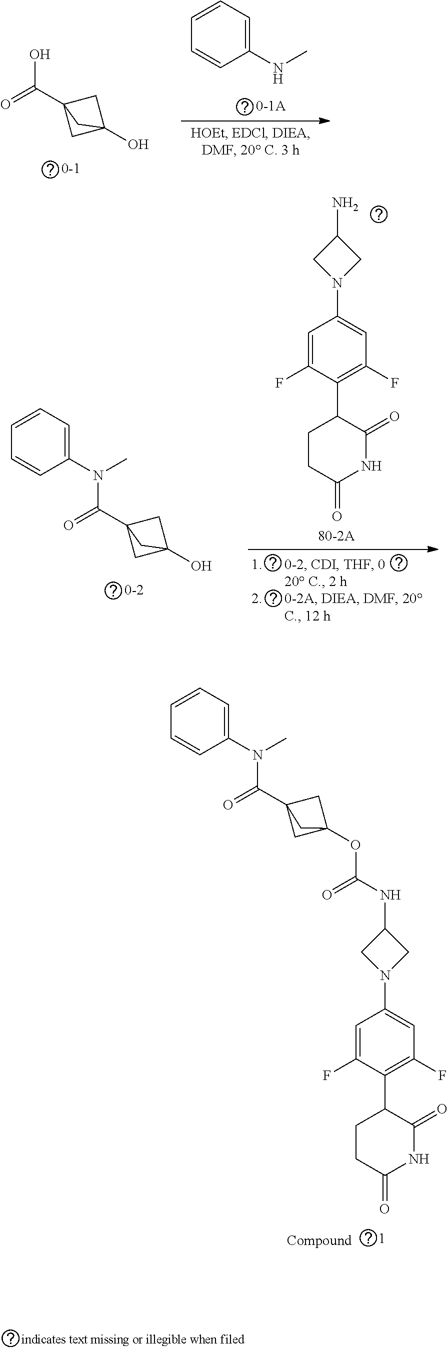

### Step 1. Procedure for Compound 2—3-hydroxy-N-methyl-N-phenylbicyclo[1.1.1]pentane-1-carboxamide

To a solution of 3-hydroxybicyclo[1.1.1]pentane-1-carboxylic acid (100 mg, 780 umol, 1.00 eq) in N,N-dimethyl formamide (1.00 mL) were added N,N-diisopropylethylamine (201 mg, 1.56 mmol, 271 uL, 2.00 eq), 1-(3-dimethylaminopropyl)-3-ethylcarbodiimide hydrochloride (164 mg, 858 umol, 1.10 eq), 1-hydroxybenzotriazole (73.8 mg, 546 umol, 0.700 eq) and N-methylaniline (836 mg, 7.80 mmol, 847 uL, 10.0 eq). The reaction mixture was stirred at 20° C. for 3 h. The reaction was filtered. The filtrate was purified by reverse phase chromatography (C18, 40 g; condition: water/acetonitrile=100:0 to 80:20, 0.1% formic acid) and lyophilized to afford 3-hydroxy-N-methyl-N-phenylbicyclo[1.1.1]pentane-1-carboxamide (61.0 mg, 280 umol, 35% yield) as a white solid.

¹H NMR (400 MHz, DMSO-d₆) δ=7.48-7.42 (m, 2H), 7.41-7.36 (m, 1H), 7.27 (d, J=7.3 Hz, 2H), 6.27-5.96 (m, 1H), 3.11 (s, 3H), 1.56 (br s, 6H).

### Step 2. Procedure for 3-(methyl(phenyl)carbamoyl)bicyclo[1.1.1]pentan-1-yl (1-(4-(2,6-dioxopiperidin-3-yl)-3,5-difluorophenyl)azetidin-3-yl)carbamate

To a solution of 3-hydroxy-N-methyl-N-phenylbicyclo[1.1.1]pentane-1-carboxamide (61.0 mg, 280 umol, 1.00 eq) in tetrahydrofuran (1.00 mL) was added di(1H-imidazol-1-yl)methanone (45.5 mg, 280 umol, 1.00 eq) at 0° C. The reaction mixture was stirred at 20° C. for 2 h. The resulting solution was added to a mixture of 3-(4-(3-aminoazetidin-1-yl)-2,6-difluorophenyl)piperidine-2,6-dione (106 mg, 273 umol, 1.00 eq, mesylate) and N,N-diisopropylethylamine (52.9 mg, 409 umol, 71.3 uL, 1.50 eq) in N,N-dimethyl formamide (1.00 mL). The reaction mixture was stirred at 20° C. for 12 h. The reaction mixture was concentrated under reduced pressure to give a residue and then filtered. The filtrate was purified by Prep-HPLC (column: Phenomenex luna C18 150*25 mm*10 um; mobile phase: [water (formic acid)-acetonitrile]; B %: 39%-69%, 58 min) and lyophilized to afford 3-(methyl(phenyl)carbamoyl)bicyclo[1.1.1]pentan-1-yl (1-(4-(2,6-dioxopiperidin-3-yl)-3,5-difluorophenyl)azetidin-3-yl)carbamate (24.9 mg, 44.39 umol, 16% yield, 96% purity) as a white solid.

¹H NMR (400 MHz, DMSO-d₆) δ=10.85 (s, 1H), 7.83 (br d, J=7.5 Hz, 1H), 7.50-7.37 (m, 3H), 7.31 (d, J=7.1 Hz, 2H), 6.11 (d, J=11.0 Hz, 2H), 4.36-4.25 (m, 1H), 4.05-3.99 (m, 3H), 3.54 (br t, J=6.8 Hz, 2H), 3.13 (s, 3H), 2.81-2.72 (m, 1H), 2.47 (br d, J=2.6 Hz, 1H), 2.09-2.03 (m, 1H), 1.95-1.89 (m, 1H), 1.89-1.76 (m, 6H). MS (ESI) m/z. 539.4 [M+H]⁺

### Example 81. Synthesis of Compound 82

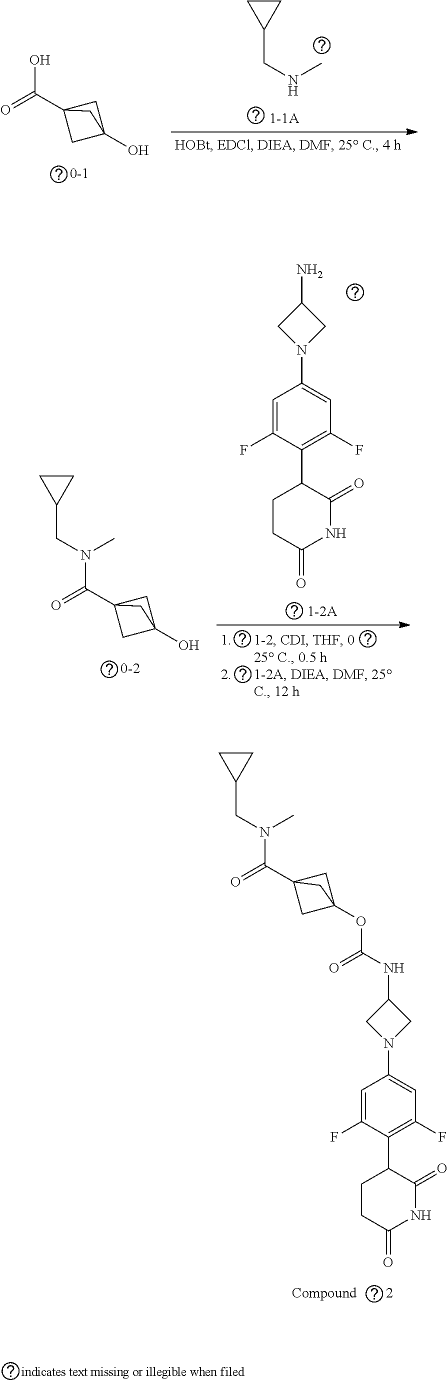

### Step 1. Procedure for Preparation of Compound 2—N-(cyclopropylmethyl)-3-hydroxy-N-methylbicyclo[1.1.1]pentane-1-carboxamide

To the solution of 3-hydroxybicyclo[1.1.1]pentane-1-carboxylic acid (100 mg, 780 umol, 1.00 eq) in dimethylformamide (3.00 mL) were added 1-Hydroxybenzotriazole (73.8 mg, 546 umol, 0.700 eq), 1-(3-dimethylaminopropyl)-3-ethylcarbodiimide hydrochloride (164 mg, 858 umol, 1.10 eq), N,N-diisopropylethylamine (202 mg, 1.56 mmol, 272 uL, 2.00 eq) and 1-cyclopropyl-N-methylmethanamine (949 mg, 7.80 mmol, 10.0 eq, hydrochloride). Then the reaction was stirred at 25° C. for 4 h. The reaction mixture was concentrated under reduced pressure to give a residue. The residue was purified by column chromatography (SiO₂, petroleum ether/ethyl acetate=0/1 to 1/2) to afford N-(cyclopropylmethyl)-3-hydroxy-N-methylbicyclo[1.1.1]pentane-1-carboxamide (80.0 mg, 205 umol, 26% yield, 50% purity) as colorless oil.

¹H NMR (400 MHz, CDCl₃) δ=4.89 (br s, 1H), 3.22 (d, J=6.8 Hz, 2H), 3.07 (s, 3H), 2.24 (s, 6H), 0.97-0.88 (m, 1H), 0.58-0.52 (m, 2H), 0.50-0.43 (m, 2H).

### Step 2. Procedure for Preparation of 3-((cyclopropylmethyl)(methyl)carbamoyl)bicyclo[1.1.1]pentan-1-yl (1-(4-(2,6-dioxopiperidin-3-yl)-3,5-difluorophenyl)azetidin-3-yl)carbamate

To the solution of N-(cyclopropylmethyl)-3-hydroxy-N-methylbicyclo[1.1.1]pentane-1-carboxamide (40.0 mg, 205 umol, 1.00 eq) in tetrahydrofuran (0.500 mL) was added di(1H-imidazol-1-yl)methanone (33.2 mg, 205 umol, 1.00 eq) at 0° C. Then the reaction was stirred at 25° C. for 0.5 h. Then to the mixture were added dimethylformamide (0.500 mL), 3-(4-(3-aminoazetidin-1-yl)-2,6-difluorophenyl)piperidine-2,6-dione (79.8 mg, 204 umol, 1.00 eq, mesylate) and N,N-diisopropylethylamine (39.5 mg, 306 umol, 53.3 uL, 1.50 eq). Then the reaction was stirred at 25° C. for 12 h. The reaction mixture was concentrated under reduced pressure to give a residue. The residue was purified by Prep-HPLC (column: Phenomenex luna C18 150*25 mm*10 um; mobile phase: [water (formic acid)-acetonitrile]; B %: 35%-65%, 9 min) and lyophilized to afford 3-((cyclopropylmethyl)(methyl)carbamoyl)bicyclo[1.1.1]pentan-1-yl (1-(4-(2,6-dioxopiperidin-3-yl)-3,5-difluorophenyl)azetidin-3-yl)carbamate (31.89 mg, 59.27 umol, 29% yield, 96% purity) as an off-white solid.

¹H NMR (400 MHz, DMSO-d₆) δ=10.85 (s, 1H), 8.00-7.94 (m, 1H), 6.15 (d, J=11.0 Hz, 2H), 4.44-4.35 (m, 1H), 4.11-4.01 (m, 3H), 3.62 (br t, J=6.7 Hz, 2H), 3.23 (br d, J=6.6 Hz, 1H), 3.14 (d, J=6.9 Hz, 1H), 3.05 (s, 2H), 2.85 (s, 1H), 2.81-2.73 (m, 1H), 2.37 (d, J=4.6 Hz, 7H), 2.09-2.02 (m, 1H), 1.96-1.91 (m, 1H), 0.94-0.84 (m, 1H), 0.51-0.40 (m, 2H), 0.25-0.17 (m, 2H).

¹H NMR (400 MHz, DMSO-d₆) δ=10.56 (br s, 1H), 7.76-7.51 (m, 1H), 6.10 (d, J=10.9 Hz, 2H), 4.46-4.35 (m, 1H), 4.10 (t, J=7.7 Hz, 2H), 4.01 (dd, J=5.1, 12.5 Hz, 1H), 3.71-3.65 (m, 2H), 3.22 (br dd, J=2.7, 3.9 Hz, 2H), 3.09-3.07 (m, 3H), 2.81-2.72 (m, 1H), 2.56-2.52 (m, 1H), 2.38 (s, 6H), 2.15-2.05 (m, 1H), 2.02-1.96 (m, 1H), 0.95-0.86 (m, 1H), 0.48 (br d, J=1.4 Hz, 2H), 0.22 (br d, J=3.8 Hz, 2H). MS (ESI) m/z 517.4 [M+H]⁺

### Example 82. Synthesis of Compound 83

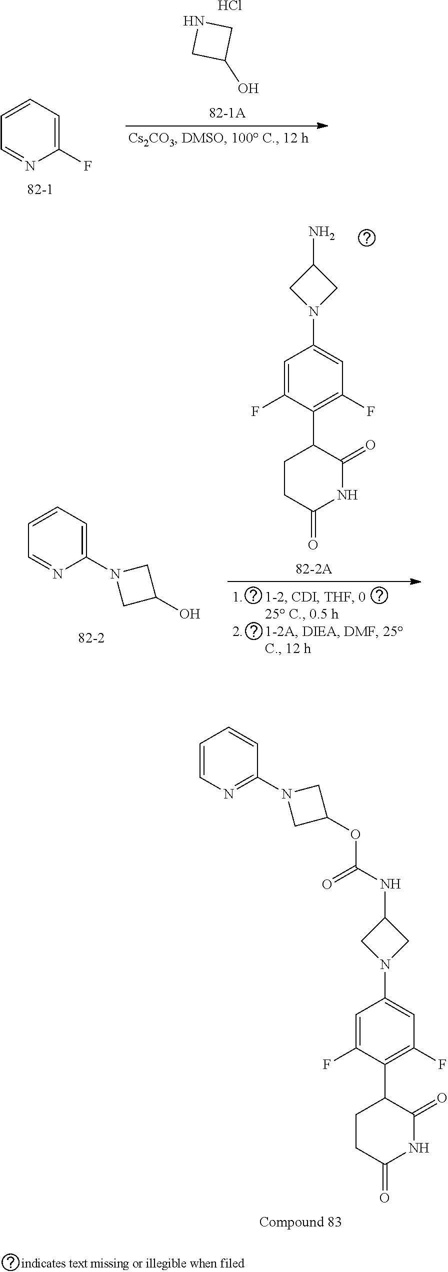 [SMILES: Cl ; OC1CNC1 ; Fc1ccccn1 ; OC1CN(c2ccccn2)C1 ; NC1CN(c2cc(F)c(C3CCC(=O)NC3=O)c(F)c2)C1 ; O=C1CCC(c2c(F)cc(N3CC(NC(=O)OC4CN(c5ccccn5)C4)C3)cc2F)C(=O)N1]

### Step 1. Procedure for Preparation of Compound 2—1-(pyridin-2 yl)azetidin-3-ol

To a solution of 2-fluoropyridine (1.00 g, 10.3 mmol, 885 uL, 1.00 eq) in dimethylsulfoxide (10.0 mL) were added cesium carbonate (10.1 g, 30.9 mmol, 3.00 eq) and azetidin-3-ol (2.26 g, 20.6 mmol, 2.00 eq, hydrochloride). The reaction mixture was stirred at 100° C. for 12 h. The reaction mixture was filtered and the filtrate was concentrated under reduced pressure to give a residue. The residue was purified by column chromatography (SiO₂, petroleum ether/ethyl acetate=1/0 to 0/1) and concentrated under reduced pressure to afford 1-(pyridin-2-yl)azetidin-3-ol (1.00 g, 6.66 mmol, 65% yield) as yellow oil.

¹H NMR (400 MHz, DMSO-d₆) δ=8.04 (d, J=4.9 Hz, 1H), 7.48 (t, J=7.8 Hz, 1H), 6.63-6.57 (m, 1H), 6.36 (d, J=8.4 Hz, 1H), 5.61 (d, J=6.5 Hz, 1H), 4.60-4.50 (m, 1H), 4.12 (t, J=7.5 Hz, 2H), 3.63 (dd, J=4.8, 8.6 Hz, 2H).

### Step 2. Procedure for Preparation of 1-(pyridin-2-yl)azetidin-3-yl (1-(4-(2,6-dioxopiperidin-3-yl)-3,5-difluorophenyl)azetidin-3-yl)carbamate

To a solution of 1-(pyridin-2-yl)azetidin-3-ol (50.0 mg, 333 umol, 1.00 eq) in tetrahydrofuran (1.00 mL) were added di(1H-imidazol-1-yl)methanone (81.0 mg, 499 umol, 1.50 eq). The reaction mixture was stirred at 25° C. for 1 h. The resulting solution was added to a mixture of 3-(4-(3-aminoazetidin-1-yl)-2,6-difluorophenyl)piperidine-2,6-dione (128 mg, 328 umol, 1.00 eq, mesylate) and N,N-diisopropylethylamine (63.5 mg, 491 umol, 85.6 uL, 1.50 eq) in dimethylformamide (1.00 mL). The reaction mixture was stirred at 25° C. for 12 h. The reaction mixture was concentrated under reduced pressure to give a residue. The residue was purified by reverse phase chromatography (C18, 40 g, condition: water/acetonitrile=80:20 to 50:50, 0.1% formic acid) and lyophilized to afford 1-(pyridin-2-yl)azetidin-3-yl (1-(4-(2,6-dioxopiperidin-3-yl)-3,5-difluorophenyl)azetidin-3-yl)carbamate (10.56 mg, 21.95 umol, 7% yield, 98% purity) as a white solid.

¹H NMR (400 MHz, DMSO-d₆) δ=10.86 (s, 1H), 8.12 (br d, J=7.1 Hz, 1H), 8.07 (br d, J=4.9 Hz, 1H), 7.52 (br t, J=7.8 Hz, 1H), 6.65 (t, J=6.1 Hz, 1H), 6.42 (d, J=8.1 Hz, 1H), 6.15 (br d, J=11.3 Hz, 2H), 5.25-5.15 (m, 1H), 4.48-4.39 (m, 1H), 4.24 (dd, J=7.1, 8.7 Hz, 2H), 4.10 (br t, J=7.6 Hz, 2H), 4.03 (br dd, J=5.0, 12.4 Hz, 1H), 3.81 (br dd, J=3.6, 9.3 Hz, 2H), 3.65 (br t, J=6.5 Hz, 2H), 2.83-2.73 (m, 1H), 2.59-2.54 (m, 1H), 2.13-2.03 (m, 1H), 1.98-1.89 (m, 1H). MS (ESI) m/z. 472.4 [M+H]⁺

### Example 83. Synthesis of Compound 84

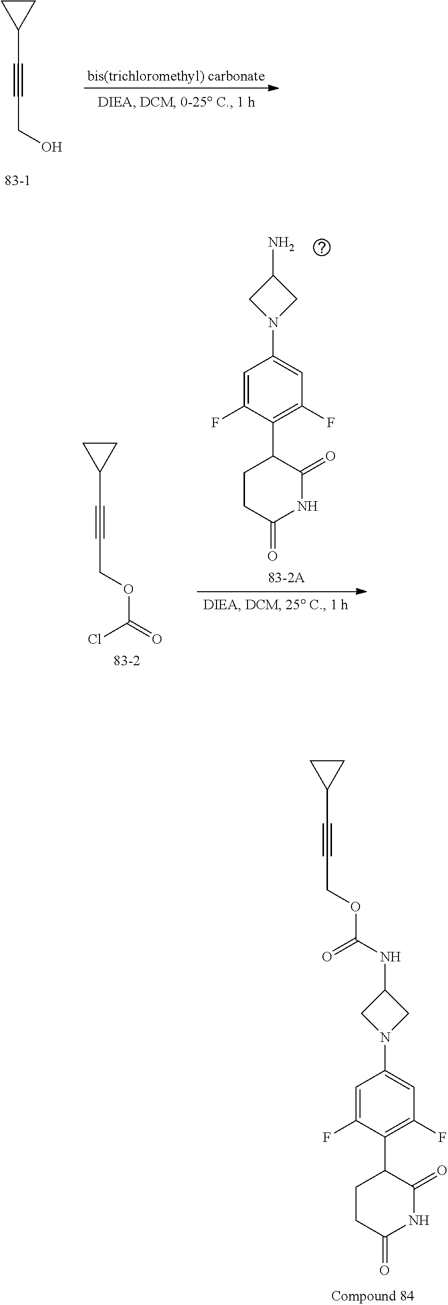 [SMILES: OCC#CC1CC1 ; "bis(trichloromethyl) carbonate" ; O=C(Cl)OCC#CC1CC1 ; NC1CN(c2cc(F)c(C3CCC(=O)NC3=O)c(F)c2)C1 ; O=C1CCC(c2c(F)cc(N3CC(NC(=O)OCC#CC4CC4)C3)cc2F)C(=O)N1]

### Step 1. Procedure for Compound 2—3-cyclopropylprop-2-yn-1-yl carbonochloridate

To a solution of 3-cyclopropylprop-2-yn-1-ol (80.0 mg, 832 umol, 1.00 eq) in dichloromethane (5.00 mL) were added N,N-diisopropylethylamine (215 mg, 1.66 mmol, 290 uL, 2.00 eq) and bis(trichloromethyl) carbonate (395 mg, 1.33 mmol, 1.60 eq) at 0° C. The mixture was stirred at 25° C. for 1 h. The reaction mixture was concentrated under reduced pressure to afford 3-cyclopropylprop-2-yn-1-yl carbonochloridate (130 mg, crude) as yellow solid.

### Step 2. Procedure for 3-cyclopropylprop-2-yn-1-yl (1-(4-(2,6-dioxopiperidin-3-yl)-3,5-difluorophenyl)azetidin-3-yl)carbamate

To a solution of 3-(4-(3-aminoazetidin-1-yl)-2,6-difluorophenyl)piperidine-2,6-dione (150 mg, 383 umol, 1.00 eq, mesylate) in dichloromethane (4.00 mL) were added N,N-diisopropylethylamine (149 mg, 1.15 mmol, 200 uL, 3.00 eq) and 3-cyclopropylprop-2-yn-1-yl carbonochloridate (91.2 mg, 575 umol, 1.50 eq) at 25° C. The mixture was stirred at 25° C. for 1 h. The reaction mixture was concentrated under reduced pressure to give a residue. The residue was purified by Prep-HPLC (column: Phenomenex luna C18 150*25 mm*10 um; mobile phase: [water (formic acid)-acetonitrile]; B %: 42%-72%, 10 min) and lyophilized to afford 3-cyclopropylprop-2-yn-1-yl (1-(4-(2,6-dioxopiperidin-3-yl)-3,5-difluorophenyl)azetidin-3-yl)carbamate (30.52 mg, 72.39 umol, 19% yield, 99% purity) as an white solid.

¹H NMR (400 MHz, DMSO-d₆) δ=10.86 (s, 1H), 8.01 (br d, J=7.6 Hz, 1H), 6.16 (d, J=11.1 Hz, 2H), 4.59 (d, J=1.8 Hz, 2H), 4.49-4.35 (m, 1H), 4.10 (t, J=7.7 Hz, 2H), 4.04 (dd, J=5.2, 12.4 Hz, 1H), 3.63 (br t, J=6.8 Hz, 2H), 2.85-2.70 (m, 1H), 2.59-2.53 (m, 1H), 2.14-2.02 (m, 1H), 1.99-1.89 (m, 1H), 1.44-1.27 (m, 1H), 0.84-0.72 (m, 2H), 0.66-0.53 (m, 2H). MS (ESI) m/z 418.3 [M+H]⁺

### Example 84. Synthesis of Compound 85

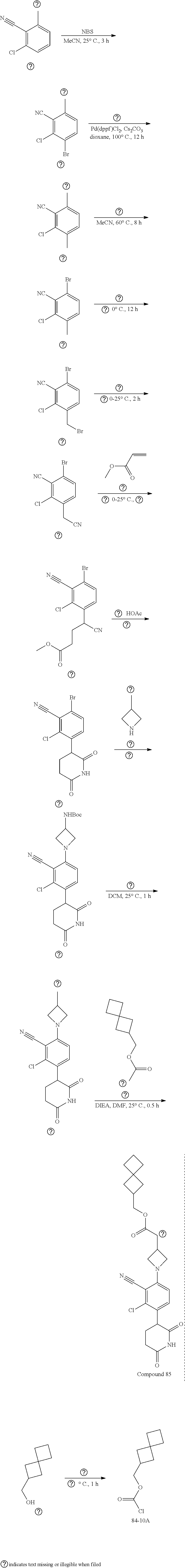

### Step 1. Procedure for Preparation of Compound 2—6-amino-3-bromo-2-chlorobenzonitrile

To a solution of 2-amino-6-chloro-benzonitrile (10.0 g, 65.5 mmol, 1.00 eq) in acetonitrile (150 mL) was added N-bromosuccinimide (11.7 g, 65.5 mmol, 1.00 eq). The reaction mixture was stirred at 25° C. for 3 h. The reaction mixture was concentrated under reduced pressure to give a residue. The residue was purified by column chromatography (SiO₂, petroleum ether/ethyl acetate=10/1 to 3/1) and concentrated under reduced pressure to afford 6-amino-3-bromo-2-chlorobenzonitrile (14.2 g, 61.4 mmol, 94% yield) as a yellow solid.

¹H NMR (400 MHz, DMSO-d₆) δ=7.57 (d, J=9.1 Hz, 1H), 6.70 (dd, J=1.1, 9.1 Hz, 1H), 6.61 (br s, 2H).

### Step 2. Procedure for Preparation of Compound 3—6-amino-2-chloro-3-methylbenzonitrile

To a solution of 6-amino-3-bromo-2-chloro-benzonitrile (14.2 g, 61.4 mmol, 1.00 eq) in dioxane (200 mL) and water (40.0 mL) were added methylboronic acid (3.67 g, 61.4 mmol, 1.00 eq), cesium carbonate (40.0 g, 123 mmol, 2.00 eq) and [1,1-bis(diphenylphosphino)ferrocene]dichloropalladium(II) (4.49 g, 6.13 mmol, 0.100 eq) under nitrogen atmosphere. The reaction was stirred at 100° C. for 12 h. The reaction mixture was filtered and the filtrate was concentrated under reduce pressure to give a residue. The residue was extracted with ethyl acetate (3×100 mL). The combined organic layers were dried over anhydrous sodium sulfate, filtered and concentrated under reduced pressure to give a crude product. The crude product was purified by column chromatography (SiO₂, petroleum ether/ethyl acetate=1/0 to 3/1) and concentrated under reduced pressure to afford 6-amino-2-chloro-3-methylbenzonitrile (7.30 g, 43.8 mmol, 71% yield) as a white solid.

¹H NMR (400 MHz, CDCl₃-d) δ=7.17 (d, J=8.4 Hz, 1H), 6.58 (d, J=8.4 Hz, 1H), 4.39 (br s, 2H), 2.27 (s, 3H).

### Step 3. Procedure for Preparation of Compound 4—6-bromo-2-chloro-3-methylbenzonitrile

To a solution of 6-amino-2-chloro-3-methyl-benzonitrile (12.0 g, 72.0 mmol, 1.00 eq) in acetonitrile (150 mL) were added cuprous bromide (15.5 g, 108 mmol, 3.29 mL, 1.50 eq) and tert-butyl nitrite (11.1 g, 108 mmol, 12.9 mL, 1.50 eq). The reaction mixture was stirred at 60° C. for 6 h under nitrogen atmosphere. The reaction mixture was filtered and the filtrate was concentrated under reduced pressure to give a residue. The residue was purified by column chromatography (SiO₂, petroleum ether/ethyl acetate=1/0 to 5/1) and concentrated under reduced pressure to afford 6-bromo-2-chloro-3-methyl-benzonitrile (5.50 g, 23.9 mmol, 33% yield) as a white solid.

¹H NMR (400 MHz, CDCl₃-d) δ=7.49 (d, J=8.3 Hz, 1H), 7.33 (dd, J=0.6, 8.3 Hz, 1H), 2.40 (s, 3H)

### Step 4. Procedure for Preparation of Compound 5—6-bromo-3-(bromomethyl)-2-chlorobenzonitrile

To a solution of 6-bromo-2-chloro-3-methyl-benzonitrile (5.50 g, 23.9 mmol, 1.00 eq) in carbon tetrachloride (90.0 mL) were added N-bromosuccinimide (4.29 g, 24.1 mmol, 1.01 eq) and (E)-2,2′-(diazene-1,2-diyl)bis(2-methylpropanenitrile) (392 mg, 2.39 mmol, 0.100 eq). The reaction was stirred at 80° C. for 12 h. The reaction mixture was concentrated under reduce pressure to give a residue. The residue was purified by column chromatography (SiO₂, petroleum ether/ethyl acetate=1/0 to 10/1) and concentrated under reduced pressure to afford 6-bromo-3-(bromomethyl)-2-chloro-benzonitrile (4.40 g, 14.2 mmol, 60% yield) as a white solid.

¹H NMR (400 MHz, CDCl₃-d) δ=7.63-7.59 (m, 1H), 7.56-7.51 (m, 1H), 4.54 (s, 2H).

### Step 5. Procedure for Preparation of Compound 6—6-bromo-2-chloro-3-(cyanomethyl)benzonitrile

To a solution of 6-bromo-3-(bromomethyl)-2-chloro-benzonitrile (4.40 g, 14.2 mmol, 1.00 eq) in acetonitrile (10.0 mL) were added trimethylsilyl cyanide (4.23 g, 42.7 mmol, 5.34 mL, 3.00 eq) and tetrabutylammonium fluoride (1.00 M, 42.7 mL, 3.00 eq) at 0° C. The reaction was stirred at 25° C. for 2 h. The reaction mixture was concentrated under reduced pressure to give a residue. The residue was purified by column chromatography (SiO₂, petroleum ether/ethyl acetate=1/0 to 0/1) and concentrated under reduced pressure to afford 6-bromo-2-chloro-3-(cyanomethyl)benzonitrile (2.50 g, 9.78 mmol, 69% yield) as a pink solid.

¹H NMR (400 MHz, CDCl₃-d) δ=7.73-7.68 (m, 1H), 7.67-7.62 (m, 1H), 3.86 (s, 2H).

### Step 6. Procedure for Preparation of Compound 7—methyl 4-(4-bromo-2-chloro-3-cyanophenyl)-4-cyanobutanoate

To a solution of 6-bromo-2-chloro-3-(cyanomethyl)benzonitrile (1.20 g, 4.70 mmol, 1.00 eq) and sodium methoxide (254 mg, 4.70 mmol, 1.00 eq) in tetrahydrofuran (240 mL) was added methyl acrylate (364 mg, 4.23 mmol, 381 uL, 0.900 eq) in tetrahydrofuran (12.0 mL) at 0° C. The reaction mixture was stirred at 20° C. for 16 h. The mixture was quenched with saturated ammonium chloride aqueous solution (50 mL) and extracted with ethyl acetate (3×50 mL). The combined organic layers were washed with brine (2×50 mL), dried over anhydrous sodium sulfate, filtered and concentrated under reduced pressure to give a residue. The residue was purified by column chromatography (SiO₂, petroleum ether/ethyl acetate=1/0 to 2/1) and concentrated under reduced pressure to afford methyl 4-(4-bromo-2-chloro-3-cyano-phenyl)-4-cyano-butanoate (900 mg, 2.63 mmol, 56% yield) as a yellow solid.

¹H NMR (400 MHz, DMSO-d₆) δ=7.97 (d, J=8.5 Hz, 1H), 7.82 (d, J=8.4 Hz, 1H), 4.64 (t, J=7.4 Hz, 1H), 3.58 (s, 3H), 2.46 (br s, 2H), 2.22-2.14 (m, 2H).

### Step 7. Procedure for Preparation of Compound 8—6-bromo-2-chloro-3-(2,6-dioxopiperidin-3-yl)benzonitrile

To a solution of methyl 4-(4-bromo-2-chloro-3-cyanophenyl)-4-cyanobutanoate (620 mg, 1.82 mmol, 1.00 eq) in acetic acid (6.00 mL) was added sulfuric acid (1.10 g, 11.3 mmol, 0.600 mL, 6.20 eq). The reaction mixture was stirred at 90° C. for 12 h. The reaction mixture was poured into ice water, and the mixture was filtered to give a brown filter cake. The filter cake was concentrated under reduced pressure to afford 6-bromo-2-chloro-3-(2,6-dioxopiperidin-3-yl)benzonitrile (298 mg, 910 umol, 50% yield) as a brown solid.

¹H NMR (400 MHz, DMSO-d₆) δ=11.02 (s, 1H), 7.90 (d, J=8.5 Hz, 1H), 7.76-7.62 (m, 1H), 4.36 (dd, J=4.9, 12.8 Hz, 1H), 2.88-2.77 (m, 1H), 2.59 (br d, J=2.8 Hz, 1H), 2.35 (dd, J=4.1, 12.9 Hz, 1H), 2.05-1.99 (m, 1H).

### Step 8. Procedure for Preparation of Compound 9—tert-butyl (1-(3-chloro-2-cyano-4-(2,6-dioxopiperidin-3-yl)phenyl)azetidin-3-yl)carbamate

To a solution of 6-bromo-2-chloro-3-(2,6-dioxo-3-piperidyl)benzonitrile (100 mg, 305 umol, 1.00 eq), tert-butyl azetidin-3-ylcarbamate (68.4 mg, 397 umol, 1.30 eq) and [1,3-bis[2,6-bis(1-propylbutyl)phenyl]-4,5-dichloro-imidazol-2-ylidene]-dichloro-(3-chloropyridin-1-ium-1-yl)palladium (29.7 mg, 30.5 umol, 0.100 eq) in dioxane (3.00 mL) was added cesium carbonate (298 mg, 916 umol, 3.00 eq). The reaction mixture was stirred at 105° C. for 16 h under nitrogen atmosphere. The reaction mixture was filtered and the filtrate was concentrated under reduced pressure to give a residue. The residue was purified by reverse phase chromatography (C18, 40 g, condition: water/acetonitrile=100:0 to 0:100, 0.1% formic acid) and lyophilized to afford tert-butyl (1-(3-chloro-2-cyano-4-(2,6-dioxopiperidin-3-yl)phenyl)azetidin-3-yl)carbamate (40.0 mg, 95.5 umol, 31% yield) as a yellow solid.

¹H NMR (400 MHz, DMSO-d₆) δ=10.88 (s, 1H), 7.59 (br d, J=3.3 Hz, 1H), 7.36 (d, J=8.9 Hz, 1H), 6.57 (d, J=8.9 Hz, 1H), 4.39 (br d, J=3.0 Hz, 3H), 4.17-4.11 (m, 1H), 3.94 (br d, J=3.5 Hz, 2H), 2.82-2.74 (m, 1H), 2.62-2.58 (m, 1H), 2.28-2.17 (m, 1H), 1.95-1.89 (m, 1H), 1.39 (s, 9H).

### Step 9. Procedure for Preparation of Compound 10—6-(3-aminoazetidin-1-yl)-2-chloro-3-(2,6-dioxopiperidin-3-yl)benzonitrile

To a solution of tert-butyl (1-(3-chloro-2-cyano-4-(2,6-dioxopiperidin-3-yl)phenyl)azetidin-3-yl)carbamate (40.0 mg, 95.5 umol, 1.00 eq) in dichloromethane (1.00 mL) was added trifluoroacetic acid (821 mg, 7.20 mmol, 533 uL, 75.4 eq). The reaction mixture was stirred at 25° C. for 1 h. The reaction mixture was concentrated under reduced pressure to afford 6-(3-aminoazetidin-1-yl)-2-chloro-3-(2,6-dioxopiperidin-3-yl)benzonitrile (30.0 mg, crude) as yellow oil. MS (ESI) m/z. 319.2 [M+H]⁺

### Step 10. Procedure for Preparation of Compound 10A—spiro[3.3]heptan-2-ylmethyl carbonochloridate

To a solution of spiro[3.3]heptan-2-ylmethanol (45.0 mg, 357 umol, 1.00 eq) in dichloromethane (1.50 mL) were added bis(trichloromethyl) carbonate (106 mg, 357 umol, 1.00 eq) and N,N-diisopropylethylamine (69.1 mg, 535 umol, 93.2 uL, 1.50 eq) at 0° C. The reaction mixture was stirred at 20° C. for 1 hr. The reaction mixture was concentrated under reduced pressure to afford spiro[3.3]heptan-2-ylmethyl carbonochloridate (67.0 mg, crude) as a white solid.

### Step 11. Procedure for preparation of spiro[3.3]heptan-2-ylmethyl (1-(3-chloro-2-cyano-4-(2,6-dioxopiperidin-3-yl)phenyl)azetidin-3-yl)carbamate

To a solution of 6-(3-aminoazetidin-1-yl)-2-chloro-3-(2,6-dioxo-3-piperidyl)benzonitrile (30.0 mg, 94.1 umol, 1.00 eq) in dimethylformamide (1.00 mL) were added N,N-diisopropylethylamine (24.3 mg, 188 umol, 32.8 uL, 2.00 eq) and spiro[3.3]heptan-2-ylmethyl carbonochloridate (35.5 mg, 188 umol, 2.00 eq). The reaction was stirred at 25° C. for 30 min. The reaction mixture was concentrated under reduced pressure to give a residue. The residue was purified by Prep-HPLC (column: Phenomenex luna C18 150*25 mm*10 um; mobile phase: [water (formic acid)-acetonitrile]; B %: 47%-77%, 10 min) and lyophilized to afford spiro[3.3]heptan-2-ylmethyl (1-(3-chloro-2-cyano-4-(2,6-dioxopiperidin-3-yl)phenyl)azetidin-3-yl)carbamate (6.66 mg, 14.00 umol, 15% yield, 99% purity) as a white solid.

¹H NMR (400 MHz, DMSO-d₆) δ=10.88 (s, 1H), 7.86 (br d, J=2.9 Hz, 1H), 7.36 (d, J=8.9 Hz, 1H), 6.57 (d, J=9.0 Hz, 1H), 4.40 (br s, 3H), 4.14 (dd, J=5.0, 12.6 Hz, 1H), 3.96 (br d, J=3.6 Hz, 2H), 3.90 (d, J=6.9 Hz, 2H), 2.82-2.71 (m, 1H), 2.53 (br d, J=4.0 Hz, 1H), 2.40-2.32 (m, 1H), 2.24 (dq, J=4.1, 13.1 Hz, 1H), 2.06-1.94 (m, 4H), 1.93-1.85 (m, 3H), 1.79-1.68 (m, 4H). MS (ESI) m/z. 471.3 [M+H]⁺

### Example 85. Synthesis of Compound 86

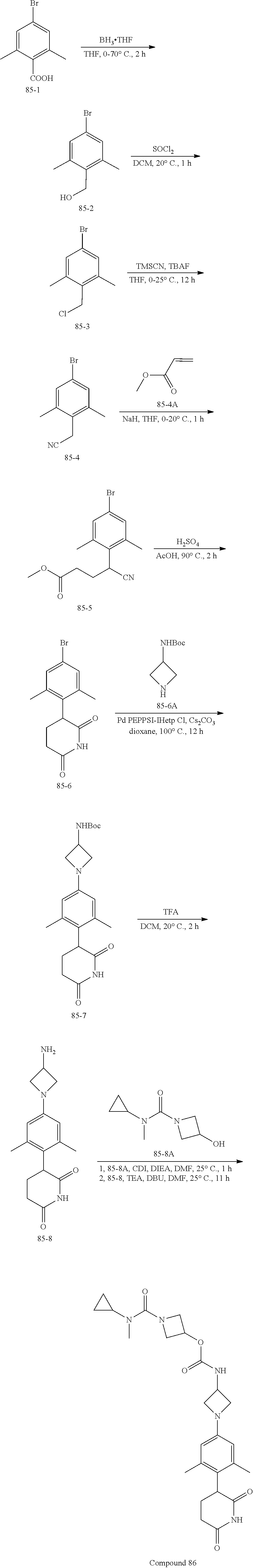

### Step 1. Procedure for Preparation of Compound 2—(4-bromo-2,6-dimethylphenyl)methanol

To a solution of 4-bromo-2,6-dimethylbenzoic acid (5.00 g, 21.8 mmol, 1.00 eq) in tetrahydrofuran (50.0 mL) was slowly added borane tetrahydrofuran (1.00 M, 109 mL, 5.00 eq) at 0° C. under nitrogen, the mixture was stirred at 0° C. for 0.5 h, then heated to 70° C. and stirred for 1.5 h under nitrogen atmosphere. The reaction mixture was quenched with methanol (50 mL) and concentrated under reduced pressure to give a residue. The residue was purified by column chromatography (SiO₂, petroleum ether/ethyl acetate=1/0 to 1/1) to afford (4-bromo-2,6-dimethylphenyl)methanol (4.30 g, 20.0 mmol, 92% yield) as a white solid.

¹H NMR (400 MHz, DMSO-d₆) δ=7.21 (s, 2H), 5.00-4.56 (m, 1H), 4.44 (s, 2H), 2.34 (s, 6H).

### Step 2. Procedure for Preparation of Compound 3—5-bromo-2-(chloromethyl)-1,3-dimethylbenzene

To a solution of (4-bromo-2,6-dimethylphenyl)methanol (4.30 g, 20.0 mmol, 1.00 eq) in dichloromethane (40.0 mL) was added thionyl chloride (12.0 g, 100 mmol, 7.25 mL, 5.00 eq). The mixture was stirred at 20° C. for 1 h. The reaction mixture was concentrated under reduced pressure to give a residue and extracted with ethyl acetate (3×50 mL). The combined organic phases were washed with brine (2×20 mL), dried over sodium sulfate, filtered and concentrated under reduced pressure to afford 5-bromo-2-(chloromethyl)-1,3-dimethylbenzene (4.60 g, crude) as yellow oil.

¹H NMR (400 MHz, DMSO-d₆) δ=7.32 (br s, 2H), 4.76 (d, J=1.8 Hz, 2H), 2.38 (s, 6H).

### Step 3. Procedure for Preparation of Compound 2—(4-bromo-2,6-dimethylphenyl)methanol

To a solution of 5-bromo-2-(chloromethyl)-1,3-dimethylbenzene (4.60 g, 19.7 mmol, 1.00 eq) in tetrahydrofuran (5.00 mL) were added trimethylsilylcyanide (3.91 g, 39.4 mmol, 4.93 mL, 2.00 eq) follow by tetrabutylammoniumfluoride (1.00 M, 39.4 mL, 2.00 eq) at 0° C., the mixture was stirred at 25° C. for 12 h. The reaction mixture was extracted with ethyl acetate (3×50 mL). The combined organic phases were washed with brine (2×30 mL), dried over sodium sulfate, filtered and concentrated under reduced pressure to give a residue. The residue was purified by column chromatography (SiO₂, petroleum ether/ethyl acetate=1/0 to 50/1) to afford 2-(4-bromo-2,6-dimethyl-phenyl)acetonitrile (3.50 g, 15.6 mmol, 79% yield) as yellow oil.

¹H NMR (400 MHz, DMSO-d₆) δ=7.34 (s, 2H), 3.88 (s, 2H), 2.34 (s, 6H).

### Step 4. Procedure for Preparation of Compound 5—5-bromo-2-(chloromethyl)-1,3-dimethylbenzene

To a solution of 2-(4-bromo-2,6-dimethylphenyl)acetonitrile (500 mg, 2.23 mmol, 1.00 eq) in tetrahydrofuran (5.00 mL) were added sodium hydride (134 mg, 3.35 mmol, 60% purity, 1.50 eq) and methyl acrylate (300 mg, 3.48 mmol, 314 uL, 1.56 eq) at 0° C., the mixture was stirred at 20° C. for 1 h. The mixture was quenched with saturated ammonium chloride aqueous solution (10 mL) and extracted with ethyl acetate (3×30 mL). The combined organic layers were dried over anhydrous sodium sulfate, filtered and concentrated under reduced pressure to give a residue. The residue was purified by column chromatography (SiO₂, petroleum ether/ethyl acetate=1/0 to 10/1) to afford methyl 4-(4-bromo-2,6-dimethylphenyl)-4-cyanobutanoate (500 mg, 1.61 mmol, 72% yield) as yellow oil.

¹H NMR (400 MHz, DMSO-d₆) δ=7.33 (s, 2H), 4.47 (dd, J=6.5, 9.8 Hz, 1H), 3.61 (s, 3H), 2.58-2.52 (m, 1H), 2.50-2.45 (m, 1H), 2.41 (s, 6H), 2.26-2.17 (m, 1H), 2.00-1.91 (m, 1H).

### Step 5. Procedure for Preparation of Compound 6—5-bromo-2-(chloromethyl)-1,3-dimethylbenzene

To a solution of methyl 4-(4-bromo-2,6-dimethylphenyl)-4-cyanobutanoate (200 mg, 645 umol, 1.00 eq) in acetic acid (5.00 mL) was added sulfuric acid (920 mg, 9.38 mmol, 0.500 mL, 14.6 eq). The mixture was stirred at 90° C. for 2 h. The reaction mixture was poured into cold water and then filtered. The filter cake was concentrated under reduced pressure to afford 3-(4-bromo-2,6-dimethylphenyl) piperidine-2,6-dione (130 mg, 439 umol, 68% yield) as a white solid.

¹H NMR (400 MHz, DMSO-d₆) δ=10.89 (s, 1H), 7.27 (s, 1H), 7.22 (s, 1H), 4.17 (dd, J=5.4, 12.8 Hz, 1H), 2.81 (ddd, J=5.8, 13.8, 16.8 Hz, 1H), 2.42 (br d, J=11.5 Hz, 1H), 2.32 (s, 3H), 2.19-2.10 (m, 1H), 2.09 (s, 3H), 1.93-1.84 (m, 1H)

### Step 6. Procedure for Preparation of Compound 7—tert-butyl (1-(4-(2,6-dioxopiperidin-3-yl)-3,5-dimethylphenyl)azetidin-3-yl)carbamate

To a solution of 3-(4-bromo-2,6-dimethylphenyl)piperidine-2,6-dione (130 mg, 439 umol, 1.00 eq) and tert-butyl azetidin-3-ylcarbamate (90.7 mg, 527 umol, 1.20 eq) in dioxane (5.00 mL) were added [1,3-bis[2,6-bis(1-propylbutyl)phenyl]-4,5-dichloro-imidazol-2-ylidene]-dichloro-(3-chloropyridin-1-ium-1-yl)palladium (8.54 mg, 8.78 umol, 0.0200 eq) and cesium carbonate (429 mg, 1.32 mmol, 3.00 eq). The mixture was stirred at 100° C. for 12 h under nitrogen atmosphere. The reaction mixture was diluted with water (20 mL) and exacted with ethyl acetate (3×20 mL). The combined organic phases were washed with brine (2×10 mL), dried over sodium sulfate, filtered and concentrated under reduced pressure to give a residue. The residue was purified by column chromatography (SiO₂, petroleum ether/ethyl acetate=1/0 to 5/1) to afford tert-butyl (1-(4-(2,6-dioxopiperidin-3-yl)-3,5-dimethylphenyl)azetidin-3-yl)carbamate (70.0 mg, 181 umol, 41% yield) as a yellow solid.

¹H NMR (400 MHz, DMSO-d₆) δ=10.76 (s, 1H), 7.50 (br d, J=7.2 Hz, 1H), 6.12 (s, 1H), 6.08 (s, 1H), 4.42-4.34 (m, 1H), 4.01 (t, J=6.8 Hz, 3H), 3.49 (br t, J=6.4 Hz, 2H), 2.84-2.76 (m, 1H), 2.47 (br s, 1H), 2.23 (s, 3H), 2.07-2.04 (m, 1H), 2.01 (s, 3H), 1.87-1.80 (m, 1H), 1.39 (s, 9H)

### Step 7. Procedure for Preparation of Compound 8—3-(4-(3-aminoazetidin-1-yl)-2,6-dimethylphenyl)piperidine-2,6-dione

To a solution of tert-butyl (1-(4-(2,6-dioxopiperidin-3-yl)-3,5-dimethylphenyl)azetidin-3-yl)carbamate (70.0 mg, 181 umol, 1.00 eq) in dichloromethane (2.50 mL) was added trifluoroacetic acid (7.06 mg, 61.9 umol, 4.59 uL, 3.43e⁻¹ eq) at 20° C. The mixture was stirred at 20° C. for 2 h. The reaction mixture was concentrated under reduced pressure to afford 3-(4-(3-aminoazetidin-1-yl)-2,6-dimethylphenyl)piperidine-2,6-dione (50.0 mg, 174 umol, 96% yield) as yellow oil. MS (ESI) m/z 287.9 [M+H]⁺

### Step 8. Procedure for Preparation of 1-(cyclopropyl(methyl)carbamoyl)azetidin-3-yl (1-(4-(2,6-dioxopiperidin-3-yl)-3,5-dimethylphenyl)azetidin-3-yl)carbamate

To a solution of N-cyclopropyl-3-hydroxy-N-methyl-azetidine-1-carboxamide (40.0 mg, 235 umol, 1.00 eq) in tetrahydrofuran (1.00 mL) was added di(1H-imidazol-1-yl)methanone (38.1 mg, 235 umol, 1.00 eq), the mixture was stirred at 25° C. for 1 h. The resulting solution was added to a mixture of 3-(4-(3-aminoazetidin-1-yl)-2,6-dimethylphenyl)piperidine-2,6-dione (50.0 mg, 174 umol, 1.00 eq), 2,3,4,6,7,8,9,10-octahydropyrimido[1,2-a]azepine (26.5 mg, 174 umol, 26.2 uL, 1.00 eq) and N,N-diisopropylethylamine (22.5 mg, 174 umol, 30.3 uL, 1.00 eq) in dimethylformamide (1.00 mL). The mixture was stirred at 25° C. for 11 h. The mixture was filtered. The filtrate was purified by Prep-HPLC (column: Phenomenex luna C18 150*25 mm*10 um; mobile phase: [water (formic acid)-acetonitrile]; B %: 23%-53%, 58 min) and lyophilized to afford 1-(cyclopropyl(methyl)carbamoyl) azetidin-3-yl(1-(4-(2,6-dioxopiperidin-3-yl)-3,5-dimethylphenyl)azetidin-3-yl)carbamate (12.57 mg, 25.5 umol, 15% yield, 97% purity) as a white solid.

¹H NMR (400 MHz, DMSO-d₆) δ=10.75 (s, 1H), 8.06 (br d, J=7.6 Hz, 1H), 6.11 (br d, J=16.0 Hz, 2H), 5.06-4.98 (m, 1H), 4.44-4.38 (m, 1H), 4.23 (dd, J=6.8, 9.6 Hz, 2H), 4.06-3.99 (m, 3H), 3.82 (br dd, J=3.8, 9.6 Hz, 2H), 3.56-3.52 (m, 2H), 2.83-2.78 (m, 1H), 2.73 (s, 3H), 2.57 (br d, J=3.4 Hz, 1H), 2.47 (br s, 1H), 2.24 (s, 3H), 2.09-2.04 (m, 1H), 2.01 (s, 3H), 1.87-1.80 (m, 1H), 0.75-0.70 (m, 2H), 0.64-0.60 (m, 2H). MS (ESI) m/z 484.3 [M+H]⁺

### Example 86. Synthesis of Compound 87

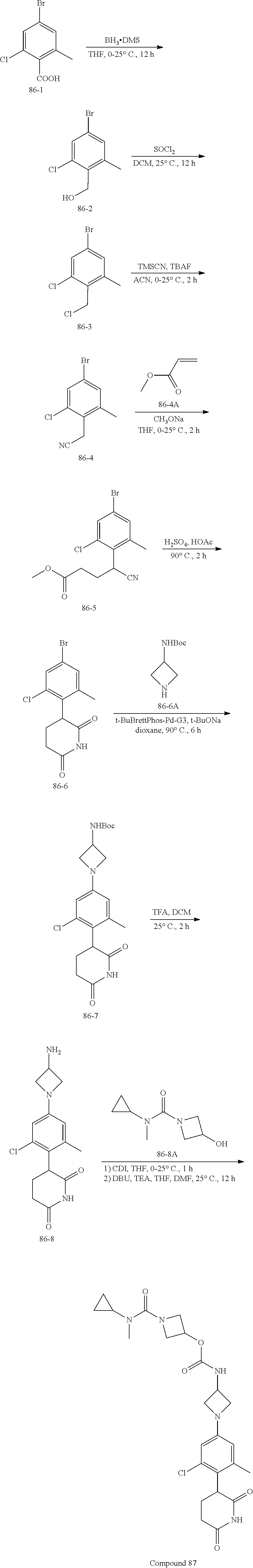

### Step 1. Procedure for Compound 2—(4-bromo-2-chloro-6-methylphenyl)methanol

To a solution of 4-bromo-2-chloro-6-methylbenzoic acid (3.00 g, 12.0 mmol, 1.00 eq) in tetrahydrofuran (30.0 mL) was added borane dimethyl sulfide complex (10.0 M, 3.61 mL, 3.00 eq) at 0° C. The mixture was stirred at 25° C. for 12 h under nitrogen atmosphere. The reaction mixture was quenched with methanol (10 mL) at 0° C. The mixture was concentrated under reduced pressure to give a residue. The residue was purified by column chromatography (SiO₂, petroleum ether/ethyl acetate=10/1 to 2/1) to afford (4-bromo-2-chloro-6-methylphenyl)methanol (2.00 g, 8.49 mmol, 70% yield) as a yellow solid.

¹H NMR (400 MHz, CDCl₃-d) δ=7.45 (d, J=1.0 Hz, 1H), 7.33 (s, 1H), 4.82 (s, 2H), 2.41 (s, 3H).

### Step 2. Procedure for Preparation of Compound 3—5-bromo-1-chloro-2-(chloromethyl)-3-methylbenzene

To a solution of (4-bromo-2-chloro-6-methylphenyl)methanol (2.00 g, 8.49 mmol, 1.00 eq) in dichloromethane (20.0 mL) was added thionyl chloride (2.02 g, 17.0 mmol, 1.23 mL, 2.00 eq). The mixture was stirred at 25° C. for 12 h under nitrogen atmosphere. The mixture was concentrated under reduced pressure to give a residue. The residue was purified by column chromatography (SiO₂, petroleum ether/ethyl acetate=10/0 to 1/1) to afford 5-bromo-1-chloro-2-(chloromethyl)-3-methylbenzene (1.00 g, 2.36 mmol, 27% yield, 60% purity) as colorless oil.

¹H NMR (400 MHz, CDCl₃-d) δ=7.45 (d, J=1.8 Hz, 1H), 7.29 (d, J=1.3 Hz, 1H), 4.73 (s, 2H), 2.46 (s, 3H).

### Step 3. Procedure for Compound 4—2-(4-bromo-2-chloro-6-methylphenyl)acetonitrile

To a solution of 5-bromo-1-chloro-2-(chloromethyl)-3-methylbenzene (800 mg, 3.15 mmol, 1.00 eq) in acetonitrile (5.00 mL) were added trimethylsilyl cyanide (938 mg, 9.45 mmol, 1.18 mL, 3.00 eq) and tetrabutylammonium fluoride (1 M, 9.45 mL, 3.00 eq) at 0° C. The mixture was stirred at 25° C. for 2 h. The mixture was diluted with water (100 mL), extracted with ethyl acetate (3×40 mL). The combined organic layer was dried over anhydrous sodium sulfate, filtered and concentrated under reduced pressure to give a residue. The residue was purified by column chromatography (SiO₂, petroleum ether/ethyl acetate=10/0 to 10/1) to afford 2-(4-bromo-2-chloro-6-methylphenyl)acetonitrile (600 mg, 2.45 mmol, 77% yield) as a white solid.

¹H NMR (400 MHz, CDCl₃-d) δ=7.49 (d, J=1.8 Hz, 1H), 7.33 (d, J=1.3 Hz, 1H), 3.81 (s, 2H), 2.45 (s, 3H).

### Step 4. Procedure for Compound 5—methyl 4-(4-bromo-2-chloro-6-methylphenyl)-4-cyanobutanoate

To a solution of 2-(4-bromo-2-chloro-6-methylphenyl)acetonitrile (600 mg, 2.45 mmol, 1.00 eq) in tetrahydrofuran (6.00 mL) were added sodium methoxide (26.5 mg, 491 umol, 0.200 eq) and methyl acrylate (232 mg, 2.70 mmol, 243 uL, 1.10 eq) at 0° C. The mixture was stirred at 25° C. for 2 h. The mixture was quenched with saturated ammonium chloride aqueous solution (10 mL). The mixture was concentrated under reduced pressure to give a residue. The residue was purified by column chromatography (SiO₂, petroleum ether/ethyl acetate=20/1 to 3/1) to afford methyl 4-(4-bromo-2-chloro-6-methylphenyl)-4-cyanobutanoate (800 mg, 2.18 mmol, 88% yield, 90% purity) as colorless oil.

¹H NMR (400 MHz, CDCl₃-d) δ=7.39 (d, J=1.9 Hz, 1H), 7.23 (d, J=1.8 Hz, 1H), 4.56 (br d, J=2.4 Hz, 1H), 3.64 (s, 3H), 2.52-2.47 (m, 2H), 2.44 (br s, 3H), 2.33 (br d, J=5.3 Hz, 1H), 2.08-1.99 (m, 1H).

### Step 5. Procedure for Compound 6—3-(4-bromo-2-chloro-6-methylphenyl)piperidine-2,6-dione

To a solution of methyl 4-(4-bromo-2-chloro-6-methylphenyl)-4-cyanobutanoate (800 mg, 2.42 mmol, 1.00 eq) in acetic acid (8.00 mL) was added sulfuric acid (1.47 g, 15.0 mmol, 0.800 mL, 6.20 eq). The mixture was stirred at 90° C. for 2 h. The reaction mixture was poured into ice water (50 mL) and filtered. The filter cake was concentrated under reduced pressure to afford 3-(4-bromo-2-chloro-6-methylphenyl)piperidine-2,6-dione (660 mg, 2.08 mmol, 86% yield) as a white solid.

¹H NMR (400 MHz, CDCl₃-d) δ=8.10 (br s, 1H), 7.44 (s, 1H), 7.32 (s, 1H), 3.99-3.92 (m, 1H), 2.85 (d, J=2.3 Hz, 1H), 2.80-2.70 (m, 1H), 2.69 (br d, J=4.3 Hz, 1H), 2.66-2.59 (m, 1H), 2.41 (s, 3H).

### Step 6. Procedure for Compound 7—tert-butyl (1-(3-chloro-4-(2,6-dioxopiperidin-3-yl)-5-methylphenyl)azetidin-3-yl)carbamate

To a solution of 3-(4-bromo-2-chloro-6-methylphenyl)piperidine-2,6-dione (250 mg, 790 umol, 1.00 eq) in dioxane (4.00 mL) were added sodium tert-butoxide (152 mg, 1.58 mmol, 2.00 eq), tert-butyl azetidin-3-ylcarbamate (272 mg, 1.58 mmol, 2.00 eq) and methanesulfonato[2-(di-tert-butylphosphino)-3,6-dimethoxy-2′,4′,6′-tri-i-propyl-1,1′-biphenyl](2′-amino-1,1′-biphenyl-2-yl)palladium(II) (67.5 mg, 790 umol, 0.100 eq). The mixture was stirred at 90° C. for 6 h under nitrogen atmosphere. The mixture was filtered and the filtrate was concentrated under reduced pressure to give a residue. The residue was purified by reverse phase chromatography (C18, 80 g; condition: water/acetonitrile=100:0 to 0:100, 0.1% formic acid) and lyophilized to afford tert-butyl (1-(3-chloro-4-(2,6-dioxopiperidin-3-yl)-5-methylphenyl)azetidin-3-yl)carbamate (65.0 mg, 159 umol, 10% yield) as a white solid.

¹H NMR (400 MHz, DMSO-d₆) δ=10.76 (s, 1H), 7.56-7.48 (m, 1H), 6.36-6.21 (m, 2H), 4.42-4.35 (m, 1H), 4.05 (br t, J=7.5 Hz, 3H), 3.54 (br t, J=6.5 Hz, 2H), 2.85-2.71 (m, 1H), 2.55 (br s, 1H), 2.39 (br s, 1H), 2.28 (s, 3H), 1.90-1.80 (m, 1H), 1.39 (s, 9H).

### Step 7. Procedure for Compound 8—3-(4-(3-aminoazetidin-1-yl)-2-chloro-6-methylphenyl)piperidine-2,6-dione

To a solution of tert-butyl (1-(3-chloro-4-(2,6-dioxopiperidin-3-yl)-5-methylphenyl)azetidin-3-yl)carbamate (65.0 mg, 159 umol, 1.00 eq) in dichloromethane (2.00 mL) was added trifluoroacetic acid (616 mg, 5.40 mmol, 0.400 mL, 33.9 eq). The mixture was stirred at 25° C. for 2 h. The mixture was filtered and the filtrate was concentrated under reduced pressure to afford 3-(4-(3-aminoazetidin-1-yl)-2-chloro-6-methylphenyl)piperidine-2,6-dione (48.0 mg, crude) as a white solid. MS (ESI) m/z 308.0 [M+H]⁺

### Step 8. Procedure for 1-(cyclopropyl(methyl)carbamoyl)azetidin-3-yl (1-(3-chloro-4-(2,6-dioxopiperidin-3-yl)-5-methylphenyl)azetidin-3-yl)carbamate

To a solution of N-cyclopropyl-3-hydroxy-N-methylazetidine-1-carboxamide (50.0 mg, 294 umol, 1.00 eq) in tetrahydrofuran (1.00 mL) was added di(1H-imidazol-1-yl)methanone (57.2 mg, 353 umol, 1.20 eq) at 0° C. The mixture was stirred at 25° C. for 1 h. The resulting solution was added to a mixture of 3-(4-(3-aminoazetidin-1-yl)-2-chloro-6-methylphenyl)piperidine-2,6-dione (48.0 mg, 156 umol, 1.00 eq), triethylamine (15.8 mg, 156 umol, 21.7 uL, 1.00 eq) and 2,3,4,6,7,8,9,10-octahydropyrimido[1,2-a]azepine (23.7 mg, 156 umol, 23.5 uL, 1.00 eq) in tetrahydrofuran (0.500 mL) and dimethylformamide (0.500 mL). The mixture was stirred at 25° C. for 12 h. The mixture was concentrated under reduced pressure to give a residue. The residue was purified by reverse phase chromatography (C18, 80 g; condition: water/acetonitrile=100:0 to 0:100, 0.1% formic acid) and lyophilized to afford 1-(cyclopropyl(methyl)carbamoyl)azetidin-3-yl (1-(3-chloro-4-(2,6-dioxopiperidin-3-yl)-5-methylphenyl)azetidin-3-yl)carbamate (11.22 mg, 22.04 umol, 14% yield, 99% purity) as a white solid.

¹H NMR (400 MHz, DMSO-d₆) δ=10.94-10.67 (m, 1H), 8.08 (d, J=7.5 Hz, 1H), 6.37-6.22 (m, 2H), 5.04-4.97 (m, 1H), 4.45-4.38 (m, 1H), 4.26-4.18 (m, 2H), 4.08 (br t, J=7.1 Hz, 2H), 3.86-3.78 (m, 2H), 3.59 (br t, J=6.6 Hz, 2H), 2.84-2.74 (m, 1H), 2.74-2.69 (m, 3H), 2.61-2.52 (m, 2H), 2.47 (br d, J=1.5 Hz, 1H), 2.34-2.25 (m, 3H), 2.13-2.05 (m, 1H), 1.90-1.78 (m, 1H), 0.75-0.68 (m, 2H), 0.65-0.58 (m, 2H).

¹H NMR (400 MHz, DMSO-d₆, T=80° C.) δ=10.56-10.39 (m, 1H), 7.84-7.70 (m, 1H), 6.31 (br s, 1H), 6.25 (s, 1H), 5.08-5.00 (m, 1H), 4.46-4.37 (m, 1H), 4.26-4.20 (m, 2H), 4.09 (t, J=7.5 Hz, 2H), 3.88-3.80 (m, 2H), 3.66 (t, J=6.6 Hz, 2H), 2.85-2.75 (m, 1H), 2.74 (s, 3H), 2.61-2.51 (m, 3H), 2.40-2.30 (m, 1H), 2.29-2.08 (m, 3H), 1.92-1.84 (m, 1H), 0.76-0.68 (m, 2H), 0.68-0.59 (m, 2H). MS (ESI) m/z 504.4 [M+H]⁺

### Example 87. Synthesis of Compound 88

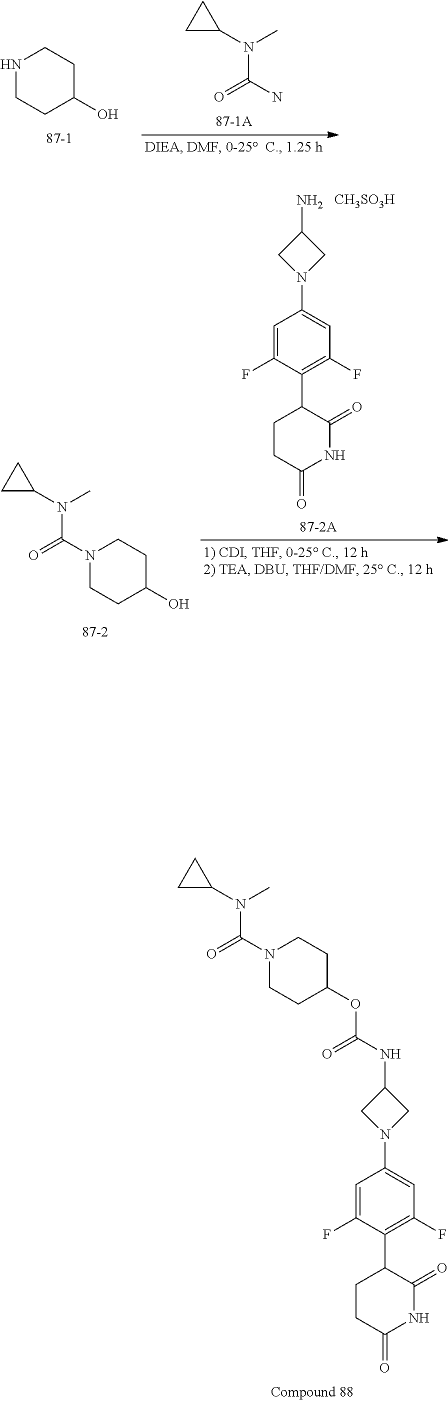

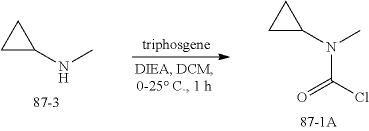

### Step 1. Procedure for Compound 1A—cyclopropyl(methyl)carbamic chloride

A solution of N-methylcyclopropanamine (500 mg, 7.03 mmol, 1.00 eq) and bis(trichloromethyl) carbonate (3.13 g, 10.6 mmol, 1.50 eq) in dichloromethane (10.0 mL) was added N,N-diisopropylethylamine (1.82 g, 14.1 mmol, 2.45 mL, 2.00 eq) at 0° C. The mixture was stirred at 25° C. for 1 h. The mixture was concentrated under reduced pressure to afford cyclopropyl(methyl)carbamic chloride (800 mg, crude) as yellow oil.

### Step 2. Procedure for Compound 2—N-cyclopropyl-4-hydroxy-N-methylpiperidine-1-carboxamide

To mixture of piperidin-4-ol (500 mg, 4.94 mmol, 1.00 eq) in dimethylformamide (2.00 mL) was added N,N-diisopropylethylamine (1.28 g, 9.89 mmol, 1.72 mL, 2.00 eq) at 0° C. for 15 min. Then cyclopropyl(methyl)carbamic chloride (726 mg, 5.44 mmol, 1.10 eq) was added and the mixture was stirred at 25° C. for 1 h. The mixture was concentrated under reduced pressure to give a residue. The residue was purified by column chromatography (SiO₂, petroleum ether/ethyl acetate=0/1) to afford N-cyclopropyl-4-hydroxy-N-methylpiperidine-1-carboxamide (560 mg, 2.77 mmol, 56% yield, 98% purity) as a white solid. 1H NMR (400 MHz, DMSO-d₆) δ=4.65 (d, J=4.2 Hz, 1H), 3.63-3.55 (m, 1H), 3.50 (td, J=4.2, 13.2 Hz, 2H), 2.83 (ddd, J=3.0, 10.1, 13.1 Hz, 2H), 2.69 (s, 3H), 2.55 (tt, J=3.6, 6.9 Hz, 1H), 1.74-1.63 (m, 2H), 1.28 (dtd, J=3.8, 9.4, 12.8 Hz, 2H), 0.65-0.58 (m, 2H), 0.48-0.40 (m, 2H).

### Step 3. Procedure for 1-(cyclopropyl(methyl)carbamoyl)piperidin-4-yl (1-(4-(2,6-dioxopiperidin-3-yl)-3,5-difluorophenyl)azetidin-3-yl)carbamate

To a solution of N-cyclopropyl-4-hydroxy-N-methylpiperidine-1-carboxamide (200 mg, 1.01 mmol, 1.00 eq) in tetrahydrofuran (5.00 mL) was added di(1H-imidazol-1-yl)methanone (196 mg, 1.21 mmol, 1.20 eq) at 0° C. The mixture was stirred at 25° C. for 12 h. The resulting solution was added to a mixture of 3-(4-(3-aminoazetidin-1-yl)-2,6-difluorophenyl)piperidine-2,6-dione (101 mg, 258 umol, 1.00 eq, mesylate), 2,3,4,6,7,8,9,10-octahydropyrimido[1,2-a]azepine (39.3 mg, 258 umol, 38.9 uL, 1.00 eq) and triethylamine (26.1 mg, 258 umol, 35.9 uL, 1.00 eq) in dimethylformamide (2.00 mL) and tetrahydrofuran (2.00 mL). The mixture was stirred at 25° C. for 12 h. The mixture was concentrated under reduced pressure to give a residue. The residue was purified by reverse phase chromatography (C18, 80 g; condition: water/acetonitrile=100:0 to 0:100, 0.1% formic acid) and lyophilized to give a crude product. The crude product was purified by column chromatography (SiO₂, petroleum ether/ethyl acetate=0/1) to afford 1-(cyclopropyl(methyl)carbamoyl)piperidin-4-yl (1-(4-(2,6-dioxopiperidin-3-yl)-3,5-difluorophenyl)azetidin-3-yl)carbamate (24.41 mg, 46.5 umol, 18% yield, 99% purity) as a white solid.

¹H NMR (400 MHz, DMSO-d₆) δ=10.85 (s, 1H), 7.85 (br d, J=7.4 Hz, 1H), 6.14 (d, J=11.0 Hz, 2H), 4.67 (td, J=4.2, 8.1 Hz, 1H), 4.48-4.33 (m, 1H), 4.09 (t, J=7.6 Hz, 2H), 4.03 (br dd, J=5.0, 12.6 Hz, 1H), 3.63 (t, J=6.8 Hz, 2H), 3.55-3.39 (m, 2H), 2.97 (br t, J=10.0 Hz, 2H), 2.82-2.74 (m, 1H), 2.71 (s, 3H), 2.60-2.55 (m, 1H), 2.54-2.51 (m, 1H), 2.07 (dq, J=4.0, 13.0 Hz, 1H), 1.98-1.90 (m, 1H), 1.82 (br d, J=9.6 Hz, 2H), 1.45 (q, J=9.0 Hz, 2H), 0.68-0.59 (m, 2H), 0.51-0.39 (m, 2H). MS (ESI) m/z. 520.2 [M+H]⁺

### Example 88. Synthesis of Compound 89

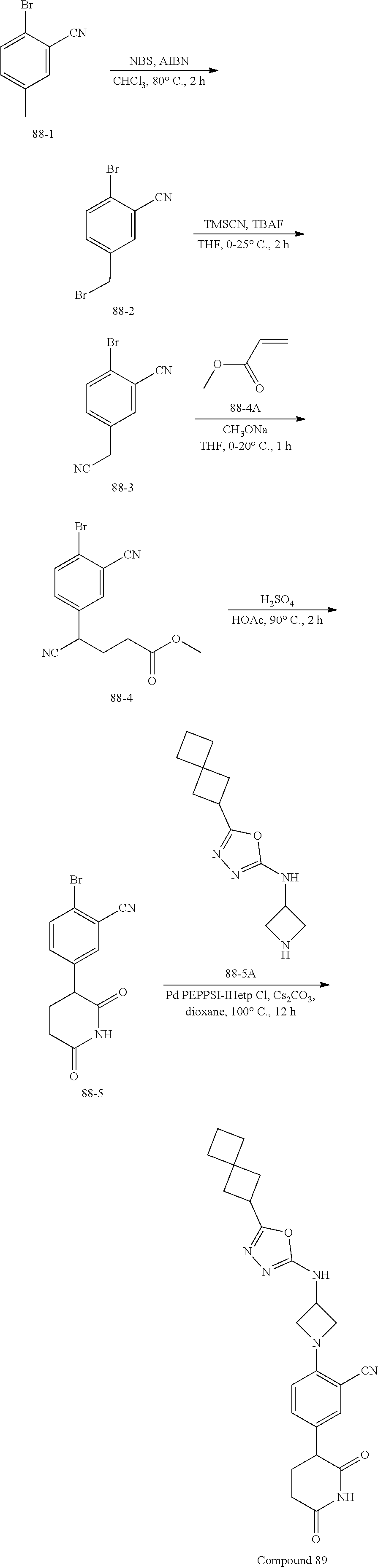

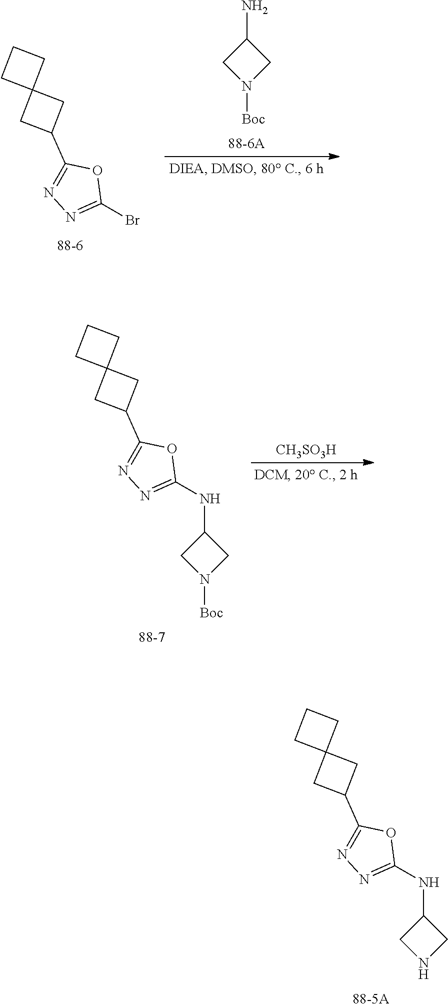 [SMILES: Brc1nnc(C2CC3(CCC3)C2)o1 ; CC(C)(C)OC(=O)N1CC(N)C1 ; CC(C)(C)OC(=O)N1CC(Nc2nnc(C3CC4(CCC4)C3)o2)C1 ; "CH3SO3H" ; C1CC2(C1)CC(c1nnc(NC3CNC3)o1)C2]

### Step 1. Procedure for Preparation of Compound 7—tert-butyl 3-((5-(spiro[3.3]heptan-2-yl)-1,3,4-oxadiazol-2-yl)amino)azetidine-1-carboxylate

To a solution of 2-bromo-5-(spiro[3.3]heptan-2-yl)-1,3,4-oxadiazole (470 mg, 1.93 mmol, 1.00 eq) and tert-butyl 3-aminoazetidine-1-carboxylate (499 mg, 2.90 mmol, 1.50 eq) in dimethylsulfoxide (7.00 mL) was added N, N-diisopropyl ethyl amine (249 mg, 1.93 mmol, 336 uL, 1.00 eq). The reaction was stirred at 80° C. for 6 h. The reaction was filtered to give a filtrate. The residue was purified by reverse phase chromatography (C18, 80 g; condition: water/acetonitrile=95:5 to 40:60, 0.1% formic) and lyophilized to afford tert-butyl 3-((5-(spiro[3.3]heptan-2-yl)-1,3,4-oxadiazol-2-yl)amino)azetidine-1-carboxylate (330 mg, 986 umol, 51% yield) as a yellow solid.

¹H NMR (400 MHz, DMSO-d₆) δ=8.04 (d, J=6.9 Hz, 1H), 4.30-4.21 (m, 1H), 4.10 (br t, J=8.0 Hz, 2H), 3.76 (br dd, J=5.1, 7.2 Hz, 2H), 3.43-3.37 (m, 1H), 2.39-2.32 (m, 2H), 2.24-2.15 (m, 2H), 2.09-2.02 (m, 2H), 1.95-1.86 (m, 2H), 1.83-1.73 (m, 2H), 1.38 (s, 9H). MS (ESI) m/z 335.0 [M+H]⁺

### Step 2. Procedure for Preparation of Compound 5A—N-(azetidin-3-yl)-5-(spiro[3.3]heptan-2-yl)-1,3,4-oxadiazol-2-amine

To a solution of tert-butyl 3-((5-(spiro[3.3]heptan-2-yl)-1,3,4-oxadiazol-2-yl)amino)azetidine-1-carboxylate (300 mg, 89.7 umol, 1.00 eq) in dichloromethane (1.00 mL) was added methanesulfonic acid (25.8 mg, 269 umol, 19.1 uL, 3.00 eq). The reaction was stirred at 20° C. for 2 h. The reaction was concentrated under reduced pressure to give N-(azetidin-3-yl)-5-(spiro[3.3]heptan-2-yl)-1,3,4-oxadiazol-2-amine (40.0 mg, 170 umol, 95% yield) as yellow oil. MS (ESI) m/z 234.9 [M+H]⁺

### Step 3. Procedure for Preparation of Compound 2—2-bromo-5-(bromomethyl)benzonitrile

To a solution of 2-bromo-5-methylbenzonitrile (6.00 g, 30.6 mmol, 1.00 eq) in chloroform (60.0 mL) was added N-bromosuccinimide (5.99 g, 33.6 mmol, 1.10 eq) and (E)-2,2′-(diazene-1,2-diyl)bis(2-methylpropanenitrile) (5.03 g, 30.6 mmol, 1.00 eq). The reaction was stirred at 80° C. for 2 h. The reaction was concentrated under reduced pressure to give a residue. The residue was purified by column chromatography (SiO₂, petroleum ether/ethyl acetate=10/1 to 5/1) to afford 2-bromo-5-(bromomethyl) benzonitrile (8.20 g, 29.8 mmol, 97% yield) as a white solid.

¹H NMR (400 MHz, DMSO-d₆) δ=8.06 (d, J=2.0 Hz, 1H), 7.90 (d, J=8.4 Hz, 1H), 7.73 (dd, J=2.4, 8.4 Hz, 1H), 4.72 (s, 2H).

### Step 4. Procedure for Preparation of Compound 3—2-bromo-5-(cyanomethyl)benzonitrile

To a solution of 2-bromo-5-(bromomethyl)benzonitrile (8.20 g, 29.8 mmol, 1.00 eq) in tetrahydrofuran (60.0 mL) was added trimethylsilanecarbonitrile (5.92 g, 59.6 mmol, 7.46 mL, 2.00 eq) and tetrabutylammonium fluoride (1.00 M, 59.6 mL, 2.00 eq) at 0° C. The reaction was stirred at 20° C. for 2 h. The reaction was concentrated under reduced pressure to give a residue. The residue was purified by column chromatography (SiO₂, petroleum ether/ethyl acetate=3/1 to 2/1) to afford 2-bromo-5-(cyanomethyl)benzonitrile (4.00 g, 18.1 mmol, 61% yield) as a white solid.

¹H NMR (400 MHz, DMSO-d₆) δ=7.97-7.90 (m, 2H), 7.65 (dd, J=2.0, 8.4 Hz, 1H), 4.11 (s, 2H).

### Step 5. Procedure for Preparation of Compound 4—methyl 4-(4-bromo-3-cyanophenyl)-4-cyanobutanoate

To a solution of 2-bromo-5-(cyanomethyl)benzonitrile (1.00 g, 4.52 mmol, 1.00 eq) in tetrahydrofuran (100 mL) was added sodium methoxide (244 mg, 4.52 mmol, 1.00 eq) and methyl acrylate (350 mg, 4.07 mmol, 366 uL, 0.900 eq) at 0° C. The reaction was stirred at 20° C. for 1 h. The reaction was diluted with water (100 mL) and extracted with ethyl acetate (3×100 mL), then the organic layers were washed with brine (100 mL), dried over anhydrous sodium sulfate, filtered and concentrated under reduced pressure to give a residue. The residue was purified by column chromatography (SiO₂, petroleum ether/ethyl acetate=10/1 to 8/1) and re-purified by reverse phase chromatography (C18, 80 g; condition: water/acetonitrile=95:5 to 40:60, 0.1% formic) and lyophilized to afford methyl 4-(4-bromo-3-cyanophenyl)-4-cyanobutanoate (300 mg, 976 umol, 22% yield) as colorless oil.

¹H NMR (400 MHz, DMSO-d₆) δ=8.02 (d, J=2.4 Hz, 1H), 7.95 (d, J=8.4 Hz, 1H), 7.70 (dd, J=2.4, 8.4 Hz, 1H), 4.39 (t, J=7.2 Hz, 1H), 3.59 (s, 3H), 2.43-2.39 (m, 2H), 2.24-2.12 (m, 2H)

### Step 6. Procedure for Preparation of Compound 5—2-bromo-5-(2,6-dioxopiperidin-3-yl)benzonitrile

To a solution of methyl 4-(4-bromo-3-cyanophenyl)-4-cyanobutanoate (300 mg, 976 umol, 1.00 eq) in sulfuric acid (300 uL) was added acetic acid (3.00 mL). The mixture was stirred at 90° C. for 2 h. The reaction was added ice water (50.0 mL) and filtered to give 2-bromo-5-(2,6-dioxopiperidin-3-yl)benzonitrile (130 mg, 443 umol, 45% yield) as a white solid

¹H NMR (400 MHz, DMSO-d₆) δ=10.92 (s, 1H), 8.02-7.75 (m, 2H), 7.55 (dd, J=2.0, 8.4 Hz, 1H), 4.00 (dd, J=4.8, 12.4 Hz, 1H), 2.71-2.65 (m, 1H), 2.60-2.57 (m, 1H), 2.35-2.26 (m, 1H), 2.07-1.99 (m, 1H).

### Step 7. Procedure for Preparation of 5-(2,6-dioxopiperidin-3-yl)-2-(3-((5-(spiro[3.3]heptan-2-yl)-1,3,4-oxadiazol-2-yl)amino)azetidin-1-yl)benzonitrile

To a solution of 2-bromo-5-(2,6-dioxopiperidin-3-yl)benzonitrile (30.0 mg, 102 umol, 1.20 eq) and N-(azetidin-3-yl)-5-(spiro[3.3]heptan-2-yl)-1,3,4-oxadiazol-2-amine (20.0 mg, 85.3 umol, 1.00 eq) in dioxane (2.00 mL) was added cesium carbonate (139 mg, 426 umol, 5.00 eq) and 1,3-bis[2,6-bis(1-propylbutyl)phenyl]-4,5-dichloro-2H-imidazol-1-ium-2-ide;3-chloropyridine;dichloropalladium (1.66 mg, 1.71 umol, 0.0200 eq). The reaction mixture was stirred at 100° C. for 2 h. The reaction mixture was filtered to give a filtrate. The filtrate was concentrated under reduced pressure to give a residue. The residue was purified by Prep-HPLC (column: Phenomenex luna C18 150*25 mm*10 um; mobile phase: [water(formic acid)-acetonitrile]; B %: 34%-64%, 10 min) and lyophilized to afford 5-(2,6-dioxopiperidin-3-yl)-2-(3-((5-(spiro[3.3]heptan-2-yl)-1,3,4-oxadiazol-2-yl)amino)azetidin-1-yl)benzonitrile (12.26 mg, 26.6 umol, 16% yield, 97% purity) as a white solid.

¹H NMR (400 MHz, DMSO-d₆) δ=10.82 (br s, 1H), 8.17 (br d, J=5.2 Hz, 1H), 7.38 (d, J=2.0 Hz, 1H), 7.32 (dd, J=2.0, 8.8 Hz, 1H), 6.61 (d, J=8.8 Hz, 1H), 4.43 (br d, J=4.0 Hz, 3H), 4.00 (br d, J=4.0 Hz, 2H), 3.78 (dd, J=4.8, 12.4 Hz, 1H), 3.46-3.42 (m, 1H), 2.71-2.62 (m, 1H), 2.55-2.52 (m, 1H), 2.38-2.33 (m, 2H), 2.24-2.16 (m, 3H), 2.08-2.04 (m, 2H), 1.98 (br dd, J=4.8, 8.0 Hz, 1H), 1.94-1.89 (m, 2H), 1.81-1.75 (m, 2H). MS (ESI) m/z 447.2 [M+H]⁺

### Example 89. Synthesis of Compound 90

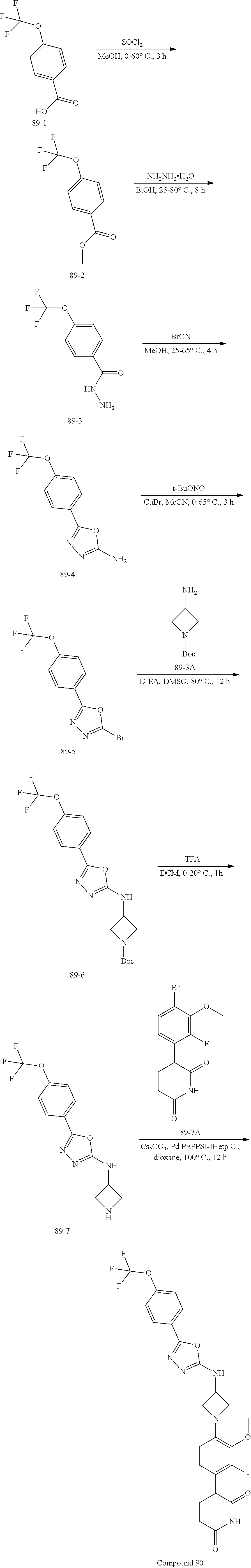

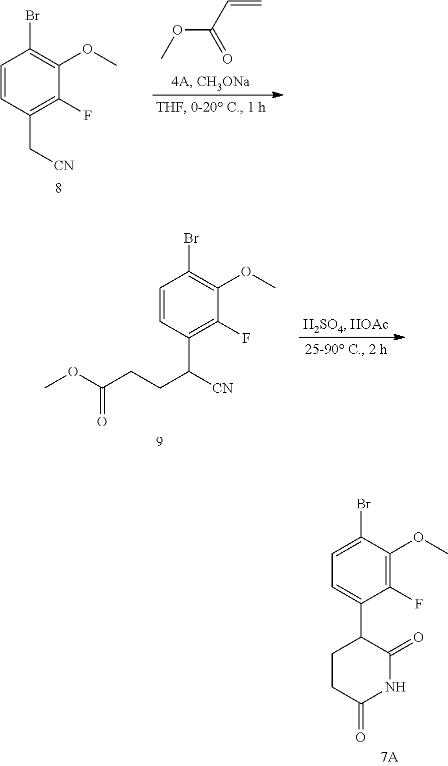

### Step 1. Procedure for Compound 1—methyl 4-(trifluoromethoxy)benzoate

To a solution of 4-(trifluoromethoxy)benzoic acid (3.00 g, 14.6 mmol, 1.00 eq) in methanol (30.0 mL) was added thionyl chloride (2.60 g, 21.8 mmol, 1.58 mL, 1.50 eq) at 0° C. The mixture was added stirred at 60° C. for 3 h. The mixture was concentrated under reduced pressure to give methyl 4-(trifluoromethoxy)benzoate (2.6 g, crude) as a white solid. MS (ESI) m/z. 220.9 [M+H]⁺.

### Step 2. Procedure for Compound 3—4-(trifluoromethoxy)benzohydrazide

To a solution of methyl 4-(trifluoromethoxy)benzoate (2.60 g, 11.8 mmol, 1.00 eq) in ethanol (30.0 mL) was added hydrazine hydrate (1.39 g, 23.6 mmol, 1.35 mL, 85% purity, 2.00 eq). The mixture was stirred at 80° C. for 8 h. The reaction mixture was cooled to 25° C. and concentrated under reduced pressure. The residue was purified by column chromatography (SiO₂, petroleum ether/ethyl acetate=0/1) to afford 4-(trifluoromethoxy)benzohydrazide (1.95 g, 8.24 mmol, 69% yield, 93% purity) as a white solid.

¹H NMR (400 MHz, DMSO-d₆) δ=9.88 (s, 1H), 8.00-7.87 (m, 2H), 7.44 (d, J=8.2 Hz, 2H), 4.53 (s, 2H).

### Step 3. Procedure for Compound 4—5-(4-(trifluoromethoxy)phenyl)-1,3,4-oxadiazol-2-amine

To a solution of 4-(trifluoromethoxy)benzohydrazide (1.20 g, 5.45 mmol, 1.00 eq) in methanol (7.00 mL) was added cyanic bromide (693 mg, 6.54 mmol, 481 uL, 1.20 eq). The mixture was stirred at 65° C. for 4 h. The reaction mixture was cooled to 25° C. The mixture was added water (60.0 mL) and extracted with ethyl acetate (2×50.0 mL). The combined layers was washed with brine (50.0 mL), dried over sodium sulfate and filtered. The filtrate was concentrated under reduced pressure to give a residue. The residue was triturated with ethyl acetate (30.0 mL) at 25° C. for 15 min and filtered. The filter cake was washed with saturated sodium bicarbonate solution and filtration to give 5-(4-(trifluoromethoxy)phenyl)-1,3,4-oxadiazol-2-amine (800 mg, 3.23 mmol, 59% yield, 99% purity) as a white solid.

¹H NMR (400 MHz, DMSO-d₆) δ=7.91 (d, J=8.9 Hz, 2H), 7.53 (br d, J=8.4 Hz, 2H), 7.32 (s, 2H).

### Step 4. Procedure for Compound 5—2-bromo-5-(4-(trifluoromethoxy)phenyl)-1,3,4-oxadiazole

To a solution of 5-(4-(trifluoromethoxy)phenyl)-1,3,4-oxadiazol-2-amine (800 mg, 3.26 mmol, 1.00 eq) in acetonitrile (5.00 mL) was added cuprous bromide (936 mg, 6.53 mmol, 199 uL, 2.00 eq) and tert-butyl nitrite (673 mg, 6.53 mmol, 776 uL, 2.00 eq) at 0° C. The mixture was stirred at 65° C. for 3 h. The residue was concentrated under reduced pressure to give a residue. The residue was purified by column chromatography (SiO₂, petroleum ether/ethyl acetate=9/1) to give 2-bromo-5-(4-(trifluoromethoxy)phenyl)-1,3,4-oxadiazole (400 mg, 1.23 mmol, 37% yield, 95% purity) as a white solid.

¹H NMR (400 MHz, DMSO-d₆) δ=8.12 (d, J=8.8 Hz, 2H), 7.61 (d, J=8.0 Hz, 2H).

### Step 5. Procedure for Compound 6—tert-butyl 3-((5-(4-(trifluoromethoxy)phenyl)-1,3,4-oxadiazol-2-yl)amino)azetidine-1-carboxylate

To a solution of 2-bromo-5-(4-(trifluoromethoxy)phenyl)-1,3,4-oxadiazole (400 mg, 1.29 mmol, 1.00 eq) in dimethylsulfoxide (2.00 mL) were N,N-diisopropylethylamine (335 mg, 2.59 mmol, 451 uL, 2.00 eq) and tert-butyl 3-aminoazetidine-1-carboxylate (245 mg, 1.42 mmol, 1.10 eq). The mixture was stirred at 80° C. for 12 h. The mixture was concentrated under reduced pressure to give a residue. The residue was purified by column chromatography (SiO₂, petroleum ether/ethyl acetate=10/0 to 3/1) to afford tert-butyl 3-((5-(4-(trifluoromethoxy)phenyl)-1,3,4-oxadiazol-2-yl)amino)azetidine-1-carboxylate (500 mg, 1.24 mmol, 95% yield, 99% purity) as a white solid.

¹H NMR (400 MHz, DMSO-d₆) δ=8.51 (d, J=6.8 Hz, 1H), 7.94 (d, J=8.8 Hz, 2H), 7.54 (br d, J=8.2 Hz, 2H), 4.43-4.34 (m, 1H), 4.16 (br t, J=8.0 Hz, 2H), 3.84 (br dd, J=5.0, 7.9 Hz, 2H), 1.39 (s, 9H).

### Step 6. Procedure for Compound 7—N-(azetidin-3-yl)-5-(4-(trifluoromethoxy)phenyl)-1,3,4-oxadiazol-2-amine

A solution of tert-butyl 3-((5-(4-(trifluoromethoxy)phenyl)-1,3,4-oxadiazol-2-yl) amino)azetidine-1-carboxylate (100 mg, 250 umol, 1.00 eq) in dichloromethane (2.50 mL) was added trifluoroacetic acid (0.500 mL) at 0° C. The mixture was stirred at 20° C. for 1 h. The mixture was concentrated under reduced pressure to give N-(azetidin-3-yl)-5-(4-(trifluoromethoxy) phenyl)-1,3,4-oxadiazol-2-amine (75.0 mg, crude) as a white solid. MS (ESI) m/z. 301.4 [M+H]⁺

### Step 7. Procedure for Compound 9—methyl 4-(4-bromo-2-fluoro-3-methoxyphenyl)-4-cyanobutanoate

A solution of 2-(4-bromo-2-fluoro-3-methoxyphenyl)acetonitrile (900 mg, 3.69 mmol, 1.00 eq and sodium methylate (19.9 mg, 369 umol, 0.100 eq) in tetrahydrofuran (5.00 mL) was added methyl acrylate (476 mg, 5.53 mmol, 498 uL, 1.50 eq) at 0° C. The mixture was stirred at 20° C. for 1 h. The reaction was quenched with saturated ammonium chloride solution (20.0 mL). The mixture was extracted with ethyl acetate (3×30.0 mL). The combined layers were washed with brine (20.0 mL), dried over sodium sulfate and filtered. The filtrate was concentrated under reduced pressure to give a residue. The residue was purified by column chromatography (SiO₂, petroleum ether/ethyl acetate=1/0 to 1/1) to afford methyl 4-(4-bromo-2-fluoro-3-methoxyphenyl)-4-cyanobutanoate (700 mg, 2.04 mmol, 55% yield, 96% purity) as a white solid.

¹H NMR (400 MHz, DMSO-d₆) δ=7.53 (br d, J=8.2 Hz, 1H), 7.17 (t, J=7.8 Hz, 1H), 4.48 (d, J=7.4 Hz, 1H), 3.89 (s, 3H), 3.57 (s, 3H), 2.44 (br t, J=7.4 Hz, 2H), 2.23-2.08 (m, 2H).

### Step 8. Procedure for Compound 7A—3-(4-bromo-2-fluoro-3-methoxyphenyl)piperidine-2,6-dione

A solution of methyl 4-(4-bromo-2-fluoro-3-methoxyphenyl)-4-cyanobutanoate (700 mg, 2.12 mmol, 1.00 eq) in acetic acid (5.00 mL) was added sulfuric acid (1.00 mL). The mixture was stirred at 90° C. for 2 h. The mixture was triturated with water (30.0 mL) at 25° C. for 15 min and filtered. The filtered cake was concentrated under reduced pressure to afford 3-(4-bromo-2-fluoro-3-methoxyphenyl)piperidine-2,6-dione (400 mg, 1.27 mmol, 59% yield) as a white solid.

¹H NMR (400 MHz, DMSO-d₆) δ=10.92 (s, 1H), 7.43 (dd, J=1.6, 8.4 Hz, 1H), 7.13-6.86 (m, 1H), 4.09 (dd, J=5.0, 12.7 Hz, 1H), 3.92-3.81 (m, 3H), 2.82-2.69 (m, 1H), 2.58-2.53 (m, 1H), 2.22 (dq, J=4.0, 13.0 Hz, 1H), 2.05-1.97 (m, 1H).

### Step 9. Procedure for 3-(2-fluoro-3-methoxy-4-(3-((5-(4-(trifluoromethoxy)phenyl)-1,3,4-oxadiazol-2-yl)amino)azetidin-1-yl)phenyl)piperidine-2,6-dione

To a mixture of N-(azetidin-3-yl)-5-(4-(trifluoromethoxy)phenyl)-1,3,4-oxadiazol-2-amine (75.0 mg, 250 umol, 1.00 eq) in dioxane (2.00 mL) were added cesium carbonate (244 mg, 749 umol, 3.00 eq), 3-(4-bromo-2-fluoro-3-methoxyphenyl)piperidine-2,6-dione (79.0 mg, 250 umol, 1.00 eq) and [1,3-bis[2,6-bis(1-propylbutyl)phenyl]-4,5-dichloro-imidazol-2-ylidene]-dichloro-(3-chloropyridin-1-ium-1-yl)palladium (24.3 mg, 25.0 umol, 0.100 eq). The mixture was stirred at 100° C. for 12 h under nitrogen atmosphere. The mixture was concentrated under reduced pressure to give a residue. The residue was purified by prep-HPLC (column: Welch Ultimate C18 150*25 mm*5 um; mobile phase: [water(FA)-ACN]; B %: 39%-69%, 10 min) and lyophilized to afford 3-(2-fluoro-3-methoxy-4-(3-((5-(4-(trifluoromethoxy)phenyl)-1,3,4-oxadiazol-2-yl)amino)azetidin-1-yl)phenyl)piperidine-2,6-dione (22.87 mg, 42.3 umol, 16% yield, 99% purity) as a white solid.

¹H NMR (400 MHz, DMSO-d₆) δ=10.82 (s, 1H), 8.57 (d, J=7.0 Hz, 1H), 7.95 (d, J=8.8 Hz, 2H), 7.55 (br d, J=8.2 Hz, 2H), 6.90-6.77 (m, 1H), 6.29 (d, J=8.8 Hz, 1H), 4.57-4.49 (m, 1H), 4.29 (t, J=7.6 Hz, 2H), 3.95-3.88 (m, 1H), 3.84 (dd, J=5.8, 7.4 Hz, 2H), 3.72 (s, 3H), 2.78-2.68 (m, 1H), 2.39 (br s, 1H), 2.19-2.11 (m, 1H), 2.00-1.94 (m, 1H). MS (ESI) m/z. 536.1 [M+H]⁺

### Example 90. Synthesis of Compound 91

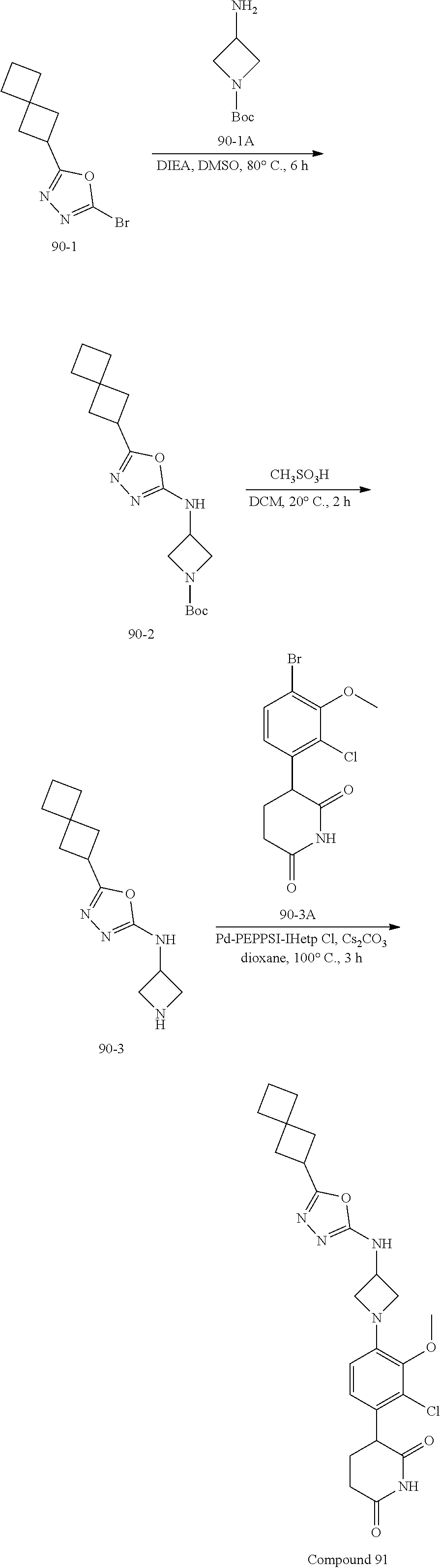

### Step 1. Procedure for Preparation of Compound 2—tert-butyl 3-((5-(spiro[3.3]heptan-2-yl)-1,3,4-oxadiazol-2-yl)amino)azetidine-1-carboxylate

To a solution of tert-butyl 3-aminoazetidine-1-carboxylate (100 mg, 411 umol, 1.00 eq) in dimethylsulfoxide (2.00 mL) was added N,N-diisopropylethylamine (159 mg, 1.23 mmol, 215 uL, 3.00 eq) and 2-bromo-5-(spiro[3.3]heptan-2-yl)-1,3,4-oxadiazole (70.9 mg, 411 umol, 1.00 eq). The reaction mixture was stirred at 80° C. for 6 h. The mixture was diluted with water (5.00 mL) and extracted with ethyl acetate (3×10 mL). The combined organic layers were washed with brine (2×10.0 mL), dried over anhydrous sodium sulfate, filtered and concentrated under reduced pressure to afford tert-butyl 3-((5-(spiro[3.3]heptan-2-yl)-1,3,4-oxadiazol-2-yl)amino)azetidine-1-carboxylate (130 mg, 389 umol, 95% yield) as yellow oil.

¹H NMR (400 MHz, DMSO-d₆) δ=8.05 (d, J=6.8 Hz, 1H), 4.30-4.19 (m, 1H), 4.09 (br s, 1H), 3.92 (br t, J=7.7 Hz, 1H), 3.75 (br d, J=2.5 Hz, 2H), 3.47-3.42 (m, 1H), 2.45-2.25 (m, 4H), 2.07-2.03 (m, 2H), 1.91 (br t, J=7.5 Hz, 2H), 1.80-1.74 (m, 2H), 1.38 (s, 9H).

### Step 2. Procedure for Preparation of Compound 3—N-(azetidin-3-yl)-5-(spiro[3.3]heptan-2-yl)-1,3,4-oxadiazol-2-amine

To a solution of tert-butyl 3-((5-(spiro[3.3]heptan-2-yl)-1,3,4-oxadiazol-2-yl)amino)azetidine-1-carboxylate (150 mg, 449 umol, 1.00 eq) in dichloromethane (2.00 mL) was added methanesulfonic acid (129 mg, 1.35 mmol, 95.8 uL, 3.00 eq). The reaction mixture was stirred at 20° C. for 2 h. The reaction mixture was concentrated under reduced pressure to afford N-(azetidin-3-yl)-5-(spiro[3.3]heptan-2-yl)-1,3,4-oxadiazol-2-amine (100 mg, 426.81 umol, 95.15% yield) as yellow oil. MS (ESI) m/z 235.1 [M+H]⁺

### Step 3. Procedure for Preparation of 3-(2-chloro-3-methoxy-4-(3-((5-(spiro[3.3]heptan-2-yl)-1,3,4-oxadiazol-2-yl)amino)azetidin-1-yl)phenyl)piperidine-2,6-dione

To a solution of 3-(4-bromo-2-chloro-3-methoxyphenyl)piperidine-2,6-dione (142 mg, 427 umol, 1.00 eq) in dioxane (5.00 mL) was added N-(azetidin-3-yl)-5-(spiro[3.3]heptan-2-yl)-1,3,4-oxadiazol-2-amine (100 mg, 427 umol, 1.00 eq), cesium carbonate (834 mg, 2.56 mmol, 6.00 eq) and 1,3-bis[2,6-bis(1-propylbutyl)phenyl]-4,5-dichloro-2H-imidazol-1-ium-2-ide;3-chloropyridine;dichloropalladium (41.5 mg, 42.7 umol, 0.100 eq). The reaction mixture was stirred at 100° C. for 3 h. The reaction mixture was filtered. The filtrate was purified by column chromatography (SiO₂, dichloromethane:ethyl acetate=1/0 to 1/1) and concentrated under reduced pressure to give a solid. The solid was diluted with water (10.0 mL) and lyophilized to give a yellow solid. The yellow solid was triturated with petroleum ether (5.00 mL) to afford 3-(2-chloro-3-methoxy-4-(3-((5-(spiro[3.3]heptan-2-yl)-1,3,4-oxadiazol-2-yl)amino)azetidin-1-yl) phenyl)piperidine-2,6-dione (18.39 mg, 37.84 umol, 8.43% yield, 95% purity) as an off-white solid.

¹H NMR (400 MHz, DMSO-d₆) δ=10.83 (s, 1H), 8.09 (d, J=6.9 Hz, 1H), 6.91 (d, J=8.4 Hz, 1H), 6.47 (d, J=8.5 Hz, 1H), 4.40 (sxt, J=6.5 Hz, 1H), 4.24 (br t, J=7.5 Hz, 2H), 4.07 (dd, J=4.9, 11.9 Hz, 1H), 3.75 (dd, J=5.8, 7.8 Hz, 2H), 3.64 (s, 3H), 3.40 (t, J=8.4 Hz, 1H), 2.74 (ddd, J=5.3, 12.5, 17.4 Hz, 1H), 2.48 (br s, 1H), 2.39-2.33 (m, 2H), 2.28-2.17 (m, 3H), 2.06 (br t, J=7.3 Hz, 2H), 1.97 (br dd, J=4.6, 8.6 Hz, 1H), 1.92 (br t, J=7.3 Hz, 2H), 1.82-1.74 (m, 2H). MS (ESI) m/z 486.1 [M+H]⁺

### Example 91. Synthesis of Compound 92

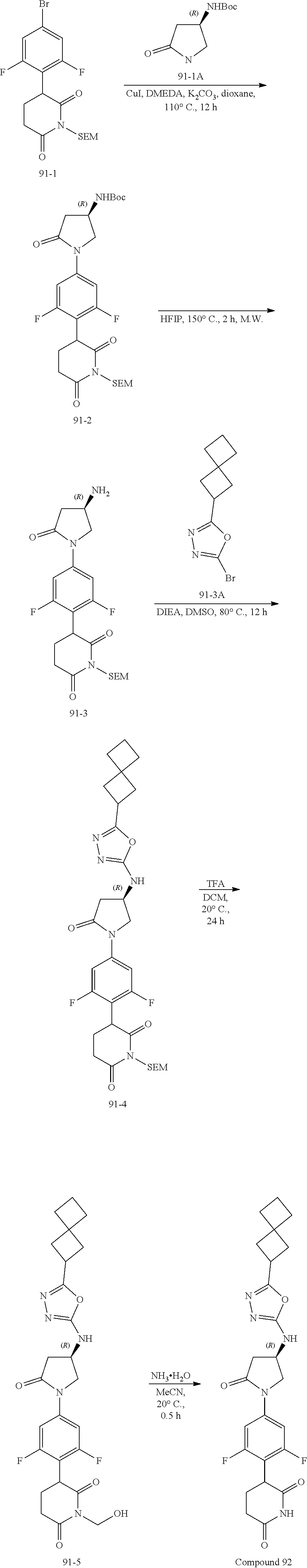

### Step 1. Procedure for Preparation of Compound 2—tert-butyl ((3R)-1-(4-(2,6-dioxo-1-((2-(trimethylsilyl)ethoxy)methyl)piperidin-3-yl)-3,5-difluorophenyl)-5-oxopyrrolidin-3-yl) carbamate

To a solution of 3-(4-bromo-2,6-difluorophenyl)-1-((2-(trimethylsilyl)ethoxy)methyl)piperidine-2,6-dione (1.74 g, 4.00 mmol, 1.00 eq) in dioxane (30.0 mL) was added (R)-tert-butyl (5-oxopyrrolidin-3-yl)carbamate (800 mg, 4.00 mmol, 1.00 eq), copper iodide (761 mg, 4.00 mmol, 1.00 eq), potassium carbonate (1.66 g, 12.0 mmol, 3.00 eq) and N,N-dimethylethylenediamine (352 mg, 4.00 mmol, 430 uL, 1.00 eq). The reaction was stirred at 110° C. for 12 h under nitrogen atmosphere. The reaction mixture was filtered. The filtrate was concentrated under reduced pressure and purified by column chromatography (SiO₂, petroleum ether/ethyl acetate=2/1) to afford tert-butyl ((3R)-1-(4-(2,6-dioxo-1-((2-(trimethylsilyl)ethoxy)methyl)piperidin-3-yl)-3,5-difluorophenyl)-5-oxopyrrolidin-3-yl)carbamate (1.40 g, 2.45 mmol, 61% yield, 97% purity) as colorless oil.

¹H NMR (400 MHz, DMSO-d₆) δ=7.48 (s, 1H), 7.46 (s, 1H), 5.22-5.01 (m, 2H), 4.36 (dd, J=5.1, 12.9 Hz, 1H), 4.20 (br d, J=2.0 Hz, 1H), 4.11-4.04 (m, 1H), 3.60 (dd, J=3.4, 10.1 Hz, 1H), 3.57-3.41 (m, 2H), 3.36 (br s, 1H), 3.03-2.92 (m, 1H), 2.88 (dd, J=8.2, 17.2 Hz, 1H), 2.77-2.68 (m, 1H), 2.45 (dd, J=4.5, 17.3 Hz, 1H), 2.16 (dq, J=3.5, 13.0 Hz, 1H), 2.05-1.99 (m, 1H), 1.39 (s, 9H), 0.90-0.79 (m, 2H), 0.21-0.23 (m, 9H).

### Step 2. Procedure for Preparation of Compound 3—3-(4-((R)-4-amino-2-oxopyrrolidin-1-yl)-2,6-difluorophenyl)-1-((2-(trimethylsilyl)ethoxy)methyl)piperidine-2,6-dione

tert-butyl ((3R)-1-(4-(2,6-dioxo-1-((2-(trimethylsilyl)ethoxy)methyl)piperidin-3-yl)-3,5-difluorophenyl)-5-oxopyrrolidin-3-yl)carbamate (1.00 g, 1.81 mmol, 1.00 eq) was taken up into a microwave tube in 1,1,1,3,3,3-hexafluoropropan-2-ol (15.0 mL). The sealed tube was heated at 150° C. for 2 h under microwave. The reaction mixture was concentrated under reduced pressure to afford 3-(4-((R)-4-amino-2-oxopyrrolidin-1-yl)-2,6-difluorophenyl)-1-((2-(trimethylsilyl)ethoxy)methyl)piperidine-2,6-dione (1.20 g, crude) as yellow oil. MS (ESI) m/z. 425.9 [M+H−28]⁺

### Step 3. Procedure for Preparation of Compound 4—3-(2,6-difluoro-4-((R)-2-oxo-4-((5-(spiro[3.3]heptan-2-yl)-1,3,4-oxadiazol-2-yl)amino)pyrrolidin-1-yl)phenyl)-1-((2-(trimethylsilyl)ethoxy) methyl)piperidine-2,6-dione

To a solution of 3-(4-((R)-4-amino-2-oxopyrrolidin-1-yl)-2,6-difluorophenyl)-1-((2-(trimethylsilyl)ethoxy) methyl)piperidine-2,6-dione (400 mg, 882 umol, 1.00 eq) in dimethylsulfoxide (8.00 mL) was added N,N-diisopropylethylamine (342 mg, 2.65 mmol, 461 uL, 3.00 eq) and 2-bromo-5-(spiro[3.3]heptan-2-yl)-1,3,4-oxadiazole (172 mg, 706 umol, 0.800 eq). The reaction mixture was stirred at 80° C. for 12 h. The reaction mixture was filtered. The filtrate was concentrated under reduced pressure and purified by column chromatography (SiO₂, petroleum ether/ethyl acetate=2/1 to 0/1) to afford 3-(2,6-difluoro-4-((R)-2-oxo-4-((5-(spiro[3.3]heptan-2-yl)-1,3,4-oxadiazol-2-yl)amino)pyrrolidin-1-yl)phenyl)-1-((2-(trimethylsilyl)ethoxy) methyl)piperidine-2,6-dione (180 mg, 292 umol, 33% yield) as a brown solid.

¹H NMR (400 MHz, DMSO-d₆) δ=7.99 (d, J=6.0 Hz, 1H), 7.48 (d, J=11.3 Hz, 2H), 5.10 (s, 2H), 4.39-4.34 (m, 1H), 4.33-4.23 (m, 1H), 4.16 (dd, J=6.3, 10.7 Hz, 1H), 3.84-3.77 (m, 1H), 3.56-3.49 (m, 2H), 3.40-3.39 (m, 1H), 3.03 (br d, J=9.4 Hz, 1H), 2.76 (br d, J=3.9 Hz, 1H), 2.54 (br d, J=3.5 Hz, 1H), 2.35-2.32 (m, 2H), 2.08-2.02 (m, 3H), 1.90-1.88 (m, 6H), 1.80-1.73 (m, 2H), 0.86-0.81 (m, 2H), 0.00-0.03 (m, 9H).

### Step 4. Procedure for Preparation of Compound 5—3-(2,6-difluoro-4-((R)-2-oxo-4-((5-(spiro[3.3]heptan-2-yl)-1,3,4-oxadiazol-2-yl)amino)pyrrolidin-1-yl)phenyl)-1-(hydroxymethyl) piperidine-2,6-dione

To a solution of 3-(2,6-difluoro-4-((R)-2-oxo-4-((5-(spiro[3.3]heptan-2-yl)-1,3,4-oxadiazol-2-yl)amino) pyrrolidin-1-yl)phenyl)-1-((2-(trimethylsilyl) ethoxy)methyl)piperidine-2,6-dione (180 mg, 292 umol, 1.00 eq) in dichloromethane (2.00 mL) was added trifluoroacetic acid (1.54 g, 13.5 mmol, 1.00 mL, 46.2 eq). The reaction mixture was stirred at 20° C. for 24 h. The reaction mixture was concentrated under reduced pressure to afford 3-(2,6-difluoro-4-((R)-2-oxo-4-((5-(spiro[3.3]heptan-2-yl)-1,3,4-oxadiazol-2-yl)amino)pyrrolidin-1-yl)phenyl)-1-(hydroxymethyl)piperidine-2,6-dione (150 mg, crude) as yellow oil. MS (ESI) m/z. 516.0 [M+H]⁺

### Step 5. Procedure for 3-(2,6-difluoro-4-((R)-2-oxo-4-((5-(spiro[3.3]heptan-2-yl)-1,3,4-oxadiazol-2-yl)amino)pyrrolidin-1-yl)phenyl)piperidine-2,6-dione

To a solution of 3-(2,6-difluoro-4-((R)-2-oxo-4-((5-(spiro[3.3]heptan-2-yl)-1,3,4-oxadiazol-2-yl)amino) pyrrolidin-1-yl)phenyl)-1-(hydroxymethyl)piperidine-2,6-dione (150 mg, 291 umol, 1.00 eq) in acetonitrile (1.00 mL) was added ammonium hydroxide (273 mg, 1.95 mmol, 0.300 mL, 25% purity, 6.69 eq). The reaction mixture was stirred at 20° C. for 0.5 h. The reaction mixture was diluted with N,N-dimethylformamide (1.00 mL) and filtered. The filtrate was concentrate under reduced pressure and purified by reverse phase chromatography (C18, 40 g; condition: water/acetonitrile=100:0 to 0:100, 0.1% formic acid) and lyophilized to afford 3-(2,6-difluoro-4-((R)-2-oxo-4-((5-(spiro[3.3]heptan-2-yl)-1,3,4-oxadiazol-2-yl)amino)pyrrolidin-1-yl)phenyl)piperidine-2,6-dione (15.02 mg, 30.63 umol, 11% yield, 99% purity) as a white solid.

¹H NMR (400 MHz, DMSO-d₆) δ=10.95 (s, 1H), 7.99 (d, J=6.1 Hz, 1H), 7.48 (d, J=11.3 Hz, 2H), 4.34-4.24 (m, 1H), 4.24-4.10 (m, 2H), 3.88-3.73 (m, 1H), 3.39 (t, J=8.4 Hz, 1H), 3.03 (dd, J=7.7, 17.3 Hz, 1H), 2.85-2.76 (m, 1H), 2.59 (br d, J=3.3 Hz, 1H), 2.54 (br d, J=3.1 Hz, 1H), 2.38-2.31 (m, 2H), 2.23-2.16 (m, 2H), 2.16-2.08 (m, 1H), 2.07-2.02 (m, 2H), 2.02-1.95 (m, 1H), 1.95-1.86 (m, 2H), 1.83-1.70 (m, 2H).

MS (ESI) m/z. 486.2 [M+H]⁺

### Example 92. Synthesis of Compound 93

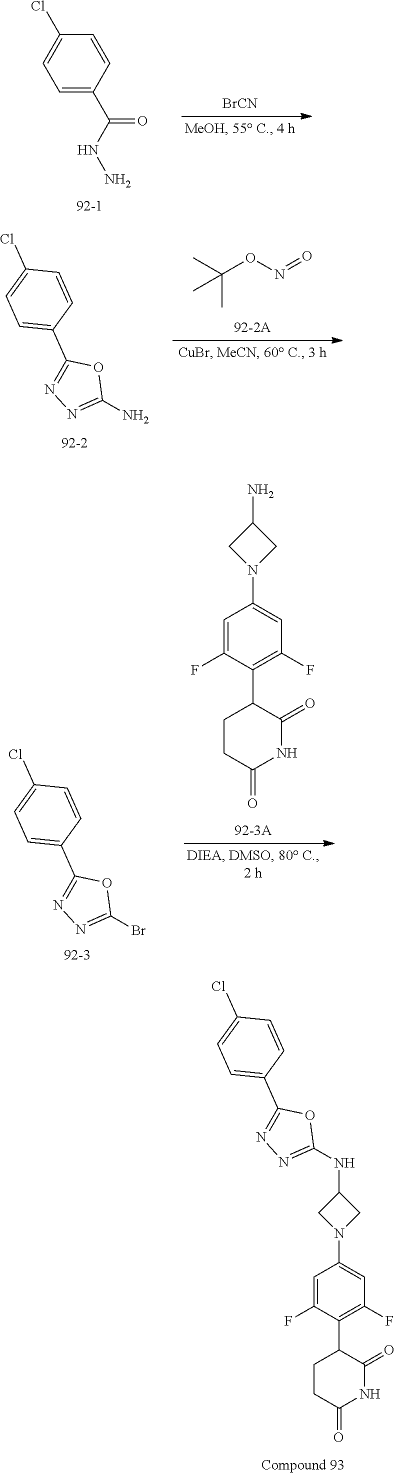

### Step 1. Procedure for Preparation of Compound 2—5-(4-chlorophenyl)-1,3,4-oxadiazol-2-amine

To a solution of 4-chlorobenzohydrazide (1.00 g, 5.86 mmol, 1.00 eq) in methanol (10.0 mL) was added cyanic bromide (745 mg, 7.03 mmol, 517 uL, 1.20 eq). The mixture was stirred at 55° C. for 4 h. The reaction mixture was cooled to 25° C. and water (40 mL) was added into the mixture. The mixture was extracted with ethyl acetate (2×30 mL). The combined organic layers were washed with brine (40.0 mL), dried over sodium sulfate and filtered. The filtrate was concentrated under reduced pressure to afford a residue. The residue was triturated with ethyl acetate (20.0 mL) and filter. The filter cake was washed with saturated sodium bicarbonate (20.0 mL) and filter to afford 5-(4-chlorophenyl)-1,3,4-oxadiazol-2-amine (850 mg, 4.35 mmol, 74% yield) as a white solid.

¹H NMR (400 MHz, DMSO-d₆) δ=7.84-7.71 (m, 2H), 7.63-7.54 (m, 2H), 7.30 (s, 2H).

### Step 2. Procedure for Preparation of Compound 3—2-bromo-5-(4-chlorophenyl)-1,3,4-oxadiazole

To a solution of 5-(4-chlorophenyl)-1,3,4-oxadiazol-2-amine (850 mg, 4.35 mmol, 1.00 eq) in acetonitrile (5.00 mL) was added cuprous bromide (935 mg, 6.52 mmol, 199 uL, 1.50 eq) and tert-butyl nitrite (672 mg, 6.52 mmol, 775 uL, 1.50 eq). The mixture was stirred at 60° C. for 3 h under nitrogen atmosphere. The reaction mixture was filtered and the filtrate was concentrated under reduced pressure to give a residue. The residue was purified by column chromatography (SiO₂, petroleum ether/ethyl acetate=1/0 to 20/1) and concentrated to afford 2-bromo-5-(4-chlorophenyl)-1,3,4-oxadiazole (900 mg, 3.47 mmol, 80% yield) as colorless oil.

¹H NMR (400 MHz, DMSO-d₆) δ=8.00 (d, J=8.8 Hz, 2H), 7.69 (d, J=8.6 Hz, 2H).

### Step 3. Procedure for Preparation of 3-(4-(3-((5-(4-chlorophenyl)-1,3,4-oxadiazol-2-yl)amino)azetidin-1-yl)-2,6-difluorophenyl)piperidine-2,6-dione

To a solution of 2-bromo-5-(4-chlorophenyl)-1,3,4-oxadiazole (105 mg, 406 umol, 1.20 eq) in dimethylsulfoxide (1.00 mL) was added 3-(4-(3-aminoazetidin-1-yl)-2,6-difluorophenyl)piperidine-2,6-dione (100 mg, 339 umol, 1.00 eq, mesylate) and N,N-diisopropylethylamine (131 mg, 1.02 mmol, 177 uL, 3.00 eq). The mixture was stirred at 80° C. for 2 h. The reaction was filtered. The filtrate was purified by Prep-HPLC (column: Welch Ultimate C18 150*25 mm*5 um; mobile phase: [water(formic acid)-acetonitrile]; B %: 34%-64%, 10 min) and lyophilized to afford 3-(4-(3-((5-(4-chlorophenyl)-1,3,4-oxadiazol-2-yl)amino)azetidin-1-yl)-2,6-difluorophenyl)piperidine-2,6-dione (38.15 mg, 75.8 umol, 22% yield, 94% purity) as a white solid.

¹H NMR (400 MHz, DMSO-d₆) δ=10.86 (s, 1H), 8.58 (br d, J=6.5 Hz, 1H), 7.82 (br d, J=8.5 Hz, 2H), 7.61 (br d, J=8.4 Hz, 2H), 6.21 (br d, J=11.3 Hz, 2H), 4.65-4.47 (m, 1H), 4.22 (br t, J=7.6 Hz, 2H), 4.05 (br dd, J=4.4, 12.4 Hz, 1H), 3.89-3.74 (m, 2H), 2.85-2.72 (m, 1H), 2.48-2.47 (m, 1H), 2.15-2.02 (m, 1H), 2.00-1.91 (m, 1H). MS (ESI) m/z 474.0 [M+H]⁺

### Example 93. Synthesis of Compound 94

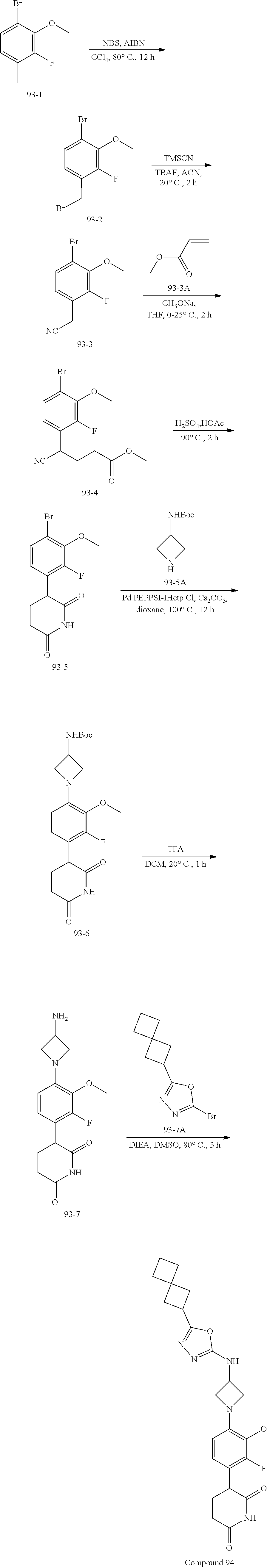

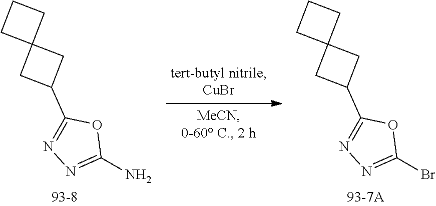 [SMILES: Nc1nnc(C2CC3(CCC3)C2)o1 ; Brc1nnc(C2CC3(CCC3)C2)o1]

### Step 1. Procedure for Preparation of Compound 7A—2-bromo-5-(spiro[3.3]heptan-2-yl)-1,3,4-oxadiazole

To a solution of 5-(spiro[3.3]heptan-2-yl)-1,3,4-oxadiazol-2-amine (100 mg, 414 umol, 1.00 eq) in acetonitrile (2.00 mL) were added tert-butyl nitrite (85.4 mg, 829 umol, 98.6 uL, 2.00 eq) and cuprous bromide (118 mg, 829 umol, 25.2 uL, 2.00 eq) at 0° C. The mixture was stirred at 60° C. for 2 h under nitrogen atmosphere. The mixture was filtered and the filtrate was concentrated under reduced pressure to give a residue. The residue was purified by prep-TLC (SiO₂, petroleum ether:ethyl acetate=10:1) to afford 2-bromo-5-(spiro[3.3]heptan-2-yl)-1,3,4-oxadiazole (20.0 mg, 82.2 umol, 19% yield) as a white solid.

### Step 2. Procedure for Preparation of Compound 2—1-bromo-4-(bromomethyl)-3-fluoro-2-methoxybenzene

To a solution of 1-bromo-3-fluoro-2-methoxy-4-methylbenzene (4.00 g, 18.3 mmol, 1.00 eq) in carbon tetrachloride (80.0 mL) was added N-bromosuccinimide (3.90 g, 21.9 mmol, 1.20 eq) and azodiisobutyronitrile (599 mg, 3.65 mmol, 0.200 eq). The mixture was stirred at 80° C. for 12 h under nitrogen atmosphere. The mixture was filtered and the filtrate was concentrated under reduced pressure to give a residue. The residue was purified by column chromatography (SiO₂, petroleum ether/ethyl acetate=1/0 to 0/1) to afford 1-bromo-4-(bromomethyl)-3-fluoro-2-methoxybenzene (4.89 g, crude) as colorless oil.

¹H NMR (400 MHz, CDCl₃) δ=7.31 (dd, J=1.1, 8.4 Hz, 1H), 7.00 (t, J=7.7 Hz, 1H), 4.47 (s, 2H), 3.98 (s, 3H).

### Step 3. Procedure for Preparation of Compound 3—2-(4-bromo-2-fluoro-3-methoxyphenyl)acetonitrile

To a solution of 1-bromo-4-(bromomethyl)-3-fluoro-2-methoxybenzene (4.89 g, 16.4 mmol, 1.00 eq) in acetonitrile (10.0 mL) was added trimethylsilanecarbonitrile (4.89 g, 49.3 mmol, 6.17 mL, 3.00 eq) and tetrabutylammonium fluoride (1.00 M, 49.3 mL, 3.00 eq). The mixture was stirred at 20° C. for 2 h. The reaction mixture was concentrated under reduced pressure to give a residue. The residue was purified by column chromatography (SiO₂, petroleum ether/ethyl acetate=1/0 to 5/1) to afford 2-(4-bromo-2-fluoro-3-methoxyphenyl)acetonitrile (2.10 g, 8.60 mmol, 52% yield) as colorless oil.

¹H NMR (400 MHz, CDCl₃) δ=7.37 (dd, J=1.9, 8.4 Hz, 1H), 7.08-7.00 (m, 1H), 3.97 (d, J=1.6 Hz, 3H), 3.73 (s, 2H).

### Step 4. Procedure for Preparation of Compound 4—methyl 4-(4-bromo-2-fluoro-3-methoxyphenyl)-4-cyanobutanoate

To a solution of 2-(4-bromo-2-fluoro-3-methoxyphenyl)acetonitrile (2.10 g, 8.60 mmol, 1.00 eq) in tetrahydrofuran (25.0 mL) was added sodium methoxide (93.0 mg, 1.72 mmol, 0.200 eq) and methyl prop-2-enoate (814 mg, 9.46 mmol, 852 uL, 1.10 eq) at 0° C. The mixture was stirred at 25° C. for 2 h. The mixture was quenched with saturated ammonium chloride aqueous solution (10.0 mL) and extracted with ethyl acetate (3×50.0 mL). The combined organic layers were dried over anhydrous sodium sulfate, filtered and concentrated under reduced pressure. The residue was purified by column chromatography (SiO₂, petroleum ether/ethyl acetate=100/1 to 1/1) to afford 4-(4-bromo-2-fluoro-3-methoxyphenyl)-4-cyanobutanoate (1.20 g, 3.63 mmol, 42% yield) as colorless oil.

¹H NMR (400 MHz, CDCl₃) δ=7.38 (dd, J=1.8, 8.4 Hz, 1H), 7.06 (dd, J=7.2, 8.3 Hz, 1H), 4.24 (t, J=7.4 Hz, 1H), 3.98 (d, J=1.5 Hz, 3H), 3.70 (s, 3H), 2.58-2.47 (m, 2H), 2.23 (dq, J=2.3, 7.3 Hz, 2H).

### Step 5. Procedure for Preparation of Compound 5—3-(4-bromo-2-fluoro-3-methoxyphenyl)piperidine-2,6-dione

To a solution of 4-(4-bromo-2-fluoro-3-methoxyphenyl)-4-cyanobutanoate (1.20 g, 3.63 mmol, 1.00 eq) in acetic acid (10.0 mL) was added sulfuric acid (1.84 g, 18.8 mmol, 1.00 mL, 5.16 eq). The mixture was stirred at 90° C. for 2 h. The crude product was triturated with water (10.0 ml) and filtered. The filter cake was washed with petroleum ether (3×5.00 mL) and concentrated under reduced pressure to afford 3-(4-bromo-2-fluoro-3-methoxyphenyl)piperidine-2,6-dione (805 mg, 2.55 mmol, 70% yield) as a white solid.

¹H NMR (400 MHz, DMSO-d₆) δ=10.92 (s, 1H), 7.42 (d, J=8.4 Hz, 1H), 7.03 (t, J=7.8 Hz, 1H), 4.09 (dd, J=5.0, 12.8 Hz, 1H), 3.85 (s, 3H), 2.79-2.69 (m, 1H), 2.58-2.52 (m, 1H), 2.21 (dq, J=4.1, 13.0 Hz, 1H), 2.05-1.97 (m, 1H).

### Step 6. Procedure for Preparation of Compound 6—tert-butyl (1-(4-(2,6-dioxopiperidin-3-yl)-3-fluoro-2-methoxyphenyl)azetidin-3-yl)carbamate

To a solution of 3-(4-bromo-2-fluoro-3-methoxyphenyl)piperidine-2,6-dione (300 mg, 949 umol, 1.00 eq) in dioxane (10.0 mL) was added tert-butyl azetidin-3-ylcarbamate (196 mg, 1.14 mmol, 1.20 eq), 1,3-bis[2,6-bis(1-propylbutyl)phenyl]-4,5-dichloro-2H-imidazol-1-ium-2-ide;3-chloropyridine;dichloropalladium (46.2 mg, 47.4 umol, 0.0500 eq) and cesium carbonate (927 mg, 2.85 mmol, 3.00 eq). The mixture was stirred at 100° C. for 12 h. The reaction mixture was concentrated under reduced pressure to give a residue. The residue was purified by column chromatography (SiO₂, petroleum ether/ethyl acetate=10/1 to 1/1) to afford tert-butyl (1-(4-(2,6-dioxopiperidin-3-yl)-3-fluoro-2-methoxyphenyl)azetidin-3-yl)carbamate (191 mg, 469 umol, 49% yield) as yellow oil.

¹H NMR (400 MHz, DMSO-d₆) δ=10.80 (s, 1H), 7.49 (br d, J=6.4 Hz, 1H), 6.80 (t, J=8.1 Hz, 1H), 6.21 (d, J=8.5 Hz, 1H), 4.40-4.30 (m, 1H), 4.12 (t, J=7.6 Hz, 2H), 3.88 (dd, J=5.0, 12.3 Hz, 1H), 3.68 (s, 3H), 3.61 (br d, J=7.0 Hz, 2H), 2.75-2.66 (m, 1H), 2.43-2.35 (m, 1H), 2.19-2.07 (m, 1H), 1.97-1.88 (m, 1H), 1.39 (s, 9H).

### Step 7. Procedure for Preparation of Compound 7—3-(4-(3-aminoazetidin-1-yl)-2-fluoro-3-methoxyphenyl)piperidine-2,6-dione

To a solution of tert-butyl (1-(4-(2,6-dioxopiperidin-3-yl)-3-fluoro-2-methoxyphenyl)azetidin-3-yl)carbamate (190 mg, 466 umol, 1.00 eq) in dichloromethane (2.00 mL) was added trifluoroacetic acid (770 mg, 6.75 mmol, 0.500 mL, 14.4 eq). The mixture was stirred at 20° C. for 1 h. The reaction mixture was concentrated under reduced pressure to afford 3-(4-(3-aminoazetidin-1-yl)-2-fluoro-3-methoxyphenyl)piperidine-2,6-dione (140 mg, crude) as yellow oil. MS (ESI) m/z 307.9 [M+H]⁺

### Step 8. Procedure for Preparation of 3-(2-fluoro-3-methoxy-4-(3-((5-(spiro[3.3]heptan-2-yl)-1,3,4-oxadiazol-2-yl)amino)azetidin-1-yl)phenyl)piperidine-2,6-dione

To a solution of 2-bromo-5-(spiro[3.3]heptan-2-yl)-1,3,4-oxadiazole (72.7 mg, 299 umol, 0.900 eq) in dimethylsulfoxide (2.00 mL) was added N,N-diisopropylethylamine (85.8 mg, 664 umol, 115 uL, 2.00 eq) and 3-(4-(3-aminoazetidin-1-yl)-2-fluoro-3-methoxyphenyl)piperidine-2,6-dione (140 mg, 332 umol, 1.00 eq, trifluoroacetic acid). The mixture was stirred at 80° C. for 12 h. The reaction mixture was concentrated under reduced pressure to give a residue. The residue was purified by prep-HPLC (column: Phenomenex luna C18 150*25 mm*10 um; mobile phase: [water(formic acid)-acetonitrile]; B %: 33%-63%, 10 min) to afford 3-(2-fluoro-3-methoxy-4-(3-((5-(spiro[3.3]heptan-2-yl)-1,3,4-oxadiazol-2-yl)amino)azetidin-1-yl)phenyl)piperidine-2,6-dione (6.00 mg, 12.6 umol, 3.81% yield, 99.0% purity) as a white solid.

¹H NMR (400 MHz, DMSO-d₆) δ=10.81 (s, 1H), 8.08 (d, J=6.9 Hz, 1H), 6.82 (t, J=8.0 Hz, 1H), 6.26 (d, J=8.4 Hz, 1H), 4.45-4.32 (m, 1H), 4.22 (t, J=7.4 Hz, 2H), 3.89 (dd, J=5.0, 12.5 Hz, 1H), 3.75 (dd, J=5.8, 7.7 Hz, 2H), 3.70 (s, 3H), 3.38 (br d, J=8.5 Hz, 1H), 2.77-2.60 (m, 2H), 2.38-2.32 (m, 2H), 2.24-2.17 (m, 2H), 2.14 (br dd, J=3.7, 13.4 Hz, 1H), 2.05 (br t, J=7.2 Hz, 2H), 1.96 (br dd, J=4.4, 9.4 Hz, 1H), 1.93-1.87 (m, 2H), 1.81-1.71 (m, 2H). MS (ESI) m/z 470.4 [M+H]⁺

### Example 94. Synthesis of Compound 95

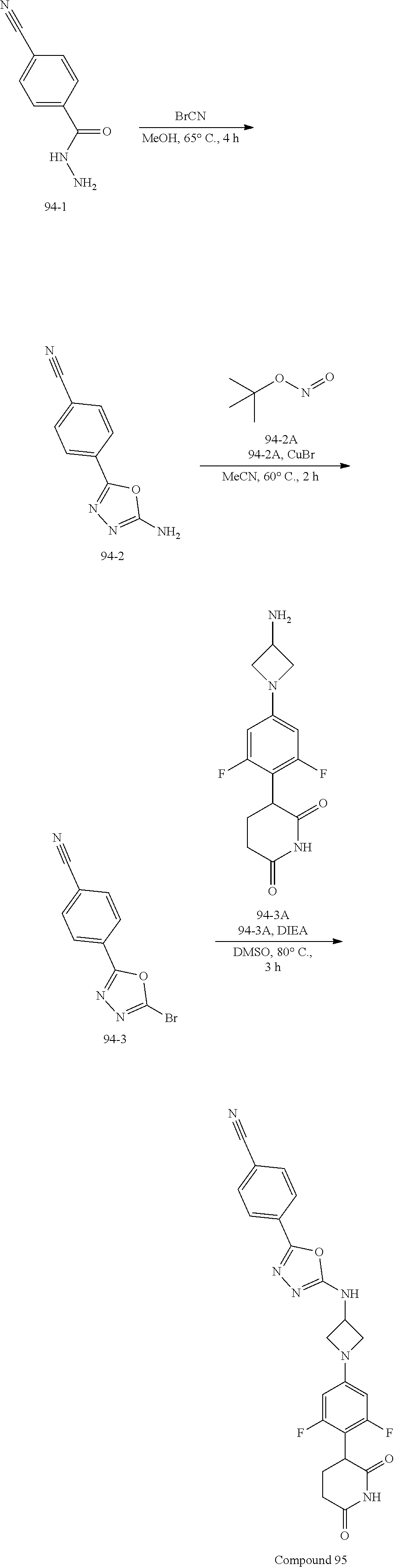

### Step 1. Procedure for Preparation of Compound 2—4-(5-amino-1,3,4-oxadiazol-2-yl)benzonitrile

To a solution of 4-cyanobenzohydrazide (1.00 g, 6.20 mmol, 1.00 eq) in methanol (10.0 mL) was added cyanogen bromide (789 mg, 7.45 mmol, 548 uL, 1.20 eq). The mixture was stirred at 65° C. for 4 h. The reaction mixture was concentrated under reduced pressure to give a residue. The residue was triturated with ethyl acetate (10.0 mL) and filtered. The filter cake was washed with ethyl acetate (10.0 mL) and dried to afford 4-(5-amino-1,3,4-oxadiazol-2-yl)benzonitrile (1.00 g, 5.37 mmol, 86% yield) as a yellow solid.

¹H NMR (400 MHz, DMSO-d₆) δ=8.01-7.97 (m, 2H), 7.96-7.92 (m, 2H), 7.49 (br s, 2H).

### Step 2. Procedure for Preparation of Compound 3—4-(5-bromo-1,3,4-oxadiazol-2-yl)benzonitrile

To a solution of tert-butyl nitrite (831 mg, 8.06 mmol, 958 uL, 1.50 eq) and cuprous bromide (1.16 g, 8.06 mmol, 245 uL, 1.50 eq) in acetonitrile (10.0 mL) was added 4-(5-amino-1,3,4-oxadiazol-2-yl)benzonitrile (1.00 g, 5.37 mmol, 1.00 eq). The mixture was stirred at 60° C. for 2 h. The reaction mixture was concentrated under reduced pressure to give a residue. The residue was purified by column chromatography (SiO₂, petroleum ether/ethyl acetate=1/0 to 10/1) to afford 4-(5-bromo-1,3,4-oxadiazol-2-yl)benzonitrile (900 mg, 3.60 mmol, 67% yield) as a yellow solid. ¹H NMR (400 MHz, DMSO-d₆) δ=8.09-8.05 (m, 2H), 8.02-7.98 (m, 2H).

### Step 3. Procedure for Preparation of 4-(5-((1-(4-(2,6-dioxopiperidin-3-yl)-3,5-difluorophenyl)azetidin-3-yl)amino)-1,3,4-oxadiazol-2-yl)benzonitrile

To a solution of 4-(5-bromo-1,3,4-oxadiazol-2-yl)benzonitrile (100 mg, 400 umol, 1.00 eq) and 3-(4-(3-aminoazetidin-1-yl)-2,6-difluorophenyl)piperidine-2,6-dione (142 mg, 480 umol, 1.20 eq) in dimethylsulfoxide (2.00 mL) was added N,N-diisopropylethylamine (155 mg, 1.20 mmol, 209 uL, 3.00 eq). The mixture was stirred at 80° C. for 3 h. The mixture was filtered and the filtrate was concentrated under reduced pressure to give a residue. The residue was purified by Prep-HPLC (column: Phenomenex luna C18 150*25 mm*10 um; mobile phase: [water(formic acid)-acetonitrile]; B %: 30%-60%, 10 min) and lyophilized to afford 4-(5-((1-(4-(2,6-dioxopiperidin-3-yl)-3,5-difluorophenyl)azetidin-3-yl)amino)-1,3,4-oxadiazol-2-yl)benzonitrile (51.13 mg, 110 umol, 28% yield, 98% purity) as a white solid

¹H NMR (400 MHz, DMSO-d₆) δ=10.87 (br s, 1H), 8.76 (br d, J=4.8 Hz, 1H), 8.05-7.94 (m, 4H), 6.22 (br d, J=11.2 Hz, 2H), 4.59 (br d, J=5.2 Hz, 1H), 4.24 (br t, J=7.6 Hz, 2H), 4.05 (br dd, J=4.6, 12.0 Hz, 1H), 3.86-3.79 (m, 2H), 2.87-2.72 (m, 1H), 2.56-2.53 (m, 1H), 2.16-2.03 (m, 1H), 2.01-1.90 (m, 1H). MS (ESI) m/z 465.2 [M+H]⁺

### Example 95. Synthesis of Compound 96

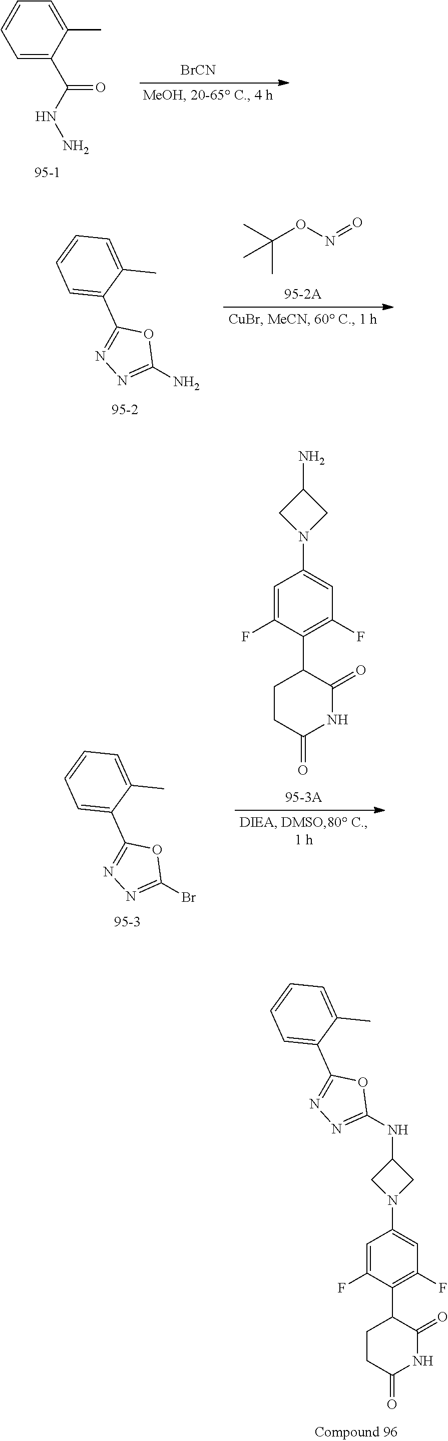

### Step 1. Procedure for Compound 2—5-(o-tolyl)-1,3,4-oxadiazol-2-amine

To a solution of 2-methylbenzohydrazide (2.00 g, 13.3 mmol, 1.00 eq) in methanol (20.0 mL) was added carbononitridic bromide (1.69 g, 16.0 mmol, 1.18 mL, 1.20 eq). The reaction mixture was stirred at 65° C. for 4 h. The reaction mixture was concentrated under reduced pressure. The solid was triturated with ethyl acetate (50.0 mL) at 20° C. for 10 min to afford 5-(o-tolyl)-1,3,4-oxadiazol-2-amine (2.00 g, 11.4 mmol, 86% yield) as a white solid.

¹H NMR (400 MHz, DMSO-d₆) δ=8.73-8.00 (m, 2H), 7.80-7.63 (m, 1H), 7.53-7.36 (m, 3H), 2.57 (s, 3H).

### Step 2. Procedure for Compound 3—2-bromo-5-(o-tolyl)-1,3,4-oxadiazole

To a solution of 5-(o-tolyl)-1,3,4-oxadiazol-2-amine (1.00 g, 5.71 mmol, 1.00 eq) in acetonitrile (10.0 mL) was added copper(II) bromide (2.55 g, 11.4 mmol, 2.00 eq) and tert-butyl nitrite (1.18 g, 11.4 mmol, 1.36 mL, 2.00 eq). The reaction mixture was stirred at 60° C. for 1 h. The reaction mixture was concentrated under reduced pressure. The residue was purified by column chromatography (SiO₂, petroleum ether/ethyl acetate=10/1 to 0/1) to afford 2-bromo-5-(o-tolyl)-1,3,4-oxadiazole (500 mg, 2.09 mmol, 37% yield) as a yellow solid.

¹H NMR (400 MHz, DMSO-d₆) δ=7.86 (d, J=7.8 Hz, 1H), 7.56-7.50 (m, 1H), 7.48-7.38 (m, 2H), 2.59 (s, 3H).

### Step 3. Procedure for 3-(2,6-difluoro-4-(3-((5-(o-tolyl)-1,3,4-oxadiazol-2-yl)amino)azetidin-1-yl)phenyl)piperidine-2,6-dione

To a solution of 2-bromo-5-(o-tolyl)-1,3,4-oxadiazole (48.9 mg, 204 umol, 0.80 eq) in dimethylsulfoxide (1.00 mL) was added 3-(4-(3-aminoazetidin-1-yl)-2,6-difluorophenyl)piperidine-2,6-dione (100 mg, 256 umol, 1.00 eq, methanesulfonic acid) and N,N-diisopropylethylamine (66.0 mg, 511 umol, 89.0 uL, 2.00 eq). The reaction mixture was stirred at 80° C. for 1 h. The reaction mixture was concentrated under reduced pressure. The residue was purified by Prep-HPLC (column: Phenomenex luna C18 150*25 mm*10 um; mobile phase: [water(formic acid)-acetonitrile]; B %: 37%-67%, 58 min) and lyophilized to afford 3-(2,6-difluoro-4-(3-((5-(0-tolyl)-1,3,4-oxadiazol-2-yl)amino)azetidin-1-yl)phenyl)piperidine-2,6-dione (27.35 mg, 59.71 umol, 23% yield, 99% purity) as a white solid.

¹H NMR (400 MHz, DMSO-d₆) δ=10.87 (br s, 1H), 8.50 (d, J=6.9 Hz, 1H), 7.72 (d, J=7.6 Hz, 1H), 7.47-7.30 (m, 3H), 6.22 (d, J=11.0 Hz, 2H), 4.64-4.48 (m, 1H), 4.23 (t, J=7.7 Hz, 2H), 4.06 (br dd, J=5.2, 12.6 Hz, 1H), 3.89-3.76 (m, 2H), 2.86-2.72 (m, 1H), 2.58 (s, 3H), 2.48-2.41 (m, 1H), 2.16-2.04 (m, 1H), 2.00-1.90 (m, 1H).

MS (ESI) m/z. 454.3 [M+H]⁺

### Example 96. Synthesis of Compound 97

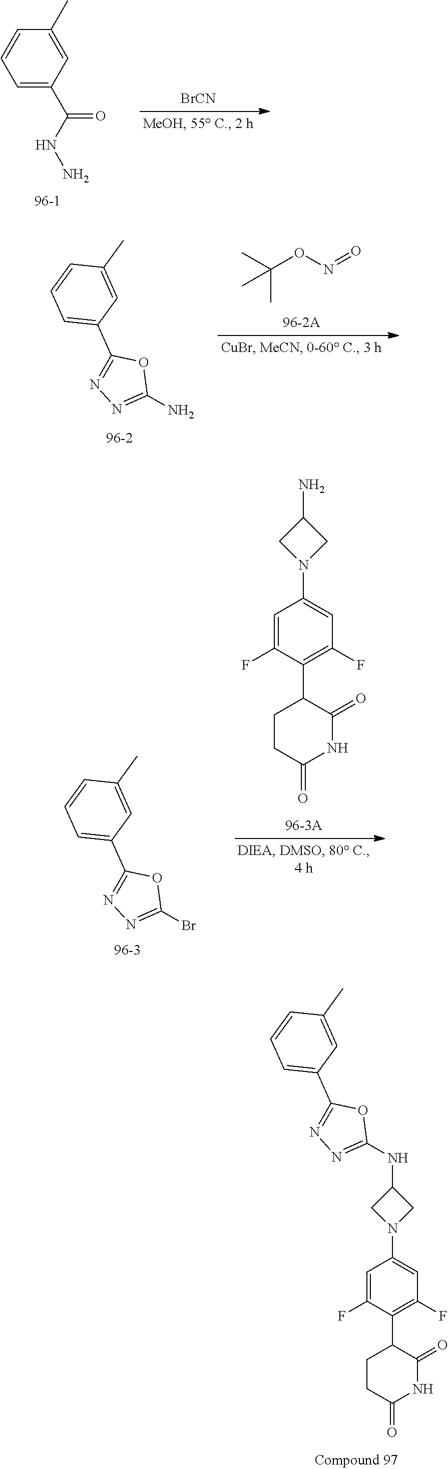

### Step 1. Procedure for Compound 2—(4-bromo-2-chloro-6-methylphenyl)methanol

To a solution of 3-methylbenzohydrazide (1.00 g, 6.66 mmol, 1.00 eq) in methanol (10.0 mL) was added cyanic bromide (846 mg, 7.99 mmol, 588 uL, 1.20 eq). The mixture was stirred at 55° C. for 2 h. The reaction mixture was cooled to 25° C. The mixture was concentrated under reduced pressure to give a residue. The residue was triturated with ethyl acetate (10.0 mL) to afford as a yellow solid. The yellow solid was neutralized with saturated sodium bicarbonate (20.0 mL) and washed with water (20.0 mL) to afford 5-(m-tolyl)-1,3,4-oxadiazol-2-amine (0.770 g, 4.40 mmol, 66% yield) as a yellow solid.

¹H NMR (400 MHz, DMSO-d₆) δ=8.57-7.88 (m, 2H), 7.67-7.58 (m, 2H), 7.49-7.42 (m, 1H), 7.41-7.35 (m, 1H), 2.38 (s, 3H).

### Step 2. Procedure for Preparation of Compound 3—2-bromo-5-(m-tolyl)-1,3,4-oxadiazole

To a solution of 5-(m-tolyl)-1,3,4-oxadiazol-2-amine (400 mg, 1.69 mmol, 1.00 eq) in acetonitrile (10.0 mL) were added tert-butyl nitrite (471 mg, 4.57 mmol, 543 uL, 2 eq) and cuprous bromide (484 mg, 3.37 mmol, 103 uL, 2.00 eq) at 0° C. The mixture was stirred at 60° C. for 3 h under nitrogen atmosphere. The mixture was filtered and the filtrate was concentrated under reduced pressure to give a residue. The residue was purified by column chromatography (SiO₂, petroleum ether/ethyl acetate=50/1 to 10/1) to afford 2-bromo-5-(m-tolyl)-1,3,4-oxadiazole (150 mg, 627 umol, 37% yield) as an white solid.

¹H NMR (400 MHz, CDCl₃) δ=7.87 (s, 1H), 7.83 (br d, J=7.1 Hz, 1H), 7.44-7.40 (m, 1H), 7.40-7.37 (m, 1H), 2.45 (s, 3H).

### Step 3. Procedure for 3-(2,6-difluoro-4-(3-((5-(m-tolyl)-1,3,4-oxadiazol-2-yl)amino)azetidin-1-yl)phenyl)piperidine-2,6-dione

To a solution of 3-(4-(3-aminoazetidin-1-yl)-2,6-difluorophenyl)piperidine-2,6-dione (270 mg, 690 umol, 1.10 eq, methanesulfonic acid) in dimethylsulfoxide (3.00 mL) were added N,N-diisopropylethylamine (162 mg, 1.25 mmol, 219 uL, 2.00 eq) and 2-bromo-5-(m-tolyl)-1,3,4-oxadiazole (150 mg, 627 umol, 1.00 eq). The mixture was stirred at 80° C. for 4 h. The mixture was filtered. The filtrate was purified by reverse phase chromatography (C18, 40 g; condition: water/acetonitrile=100:0 to 0:100, 0.1% formic acid) and lyophilized to afford 3-(2,6-difluoro-4-(3-((5-(m-tolyl)-1,3,4-oxadiazol-2-yl)amino)azetidin-1-yl)phenyl)piperidine-2,6-dione (29.75 mg, 64.95 umol, 10% yield, 99% purity) as an white solid.

¹H NMR (400 MHz, DMSO-d₆) δ=10.86 (s, 1H), 8.50 (d, J=7.0 Hz, 1H), 7.65 (s, 1H), 7.61 (br d, J=7.9 Hz, 1H), 7.42 (t, J=7.6 Hz, 1H), 7.38-7.31 (m, 1H), 6.21 (d, J=11.1 Hz, 2H), 4.63-4.51 (m, 1H), 4.22 (t, J=7.7 Hz, 2H), 4.10-4.00 (m, 1H), 3.85-3.76 (m, 2H), 2.83-2.73 (m, 1H), 2.56-2.53 (m, 1H), 2.38 (s, 3H), 2.17-2.02 (m, 1H), 1.99-1.90 (m, 1H). MS (ESI) m/z 454.4 [M+H]⁺

### Example 97. Synthesis of Compound 98

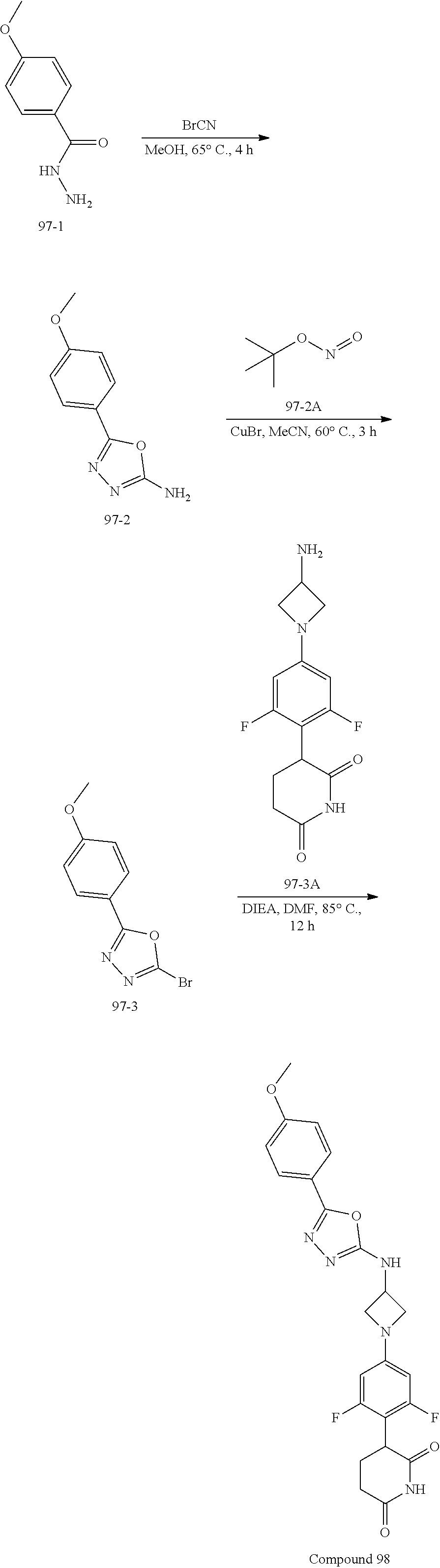

### Step 1. Procedure for Compound 1—5-(4-methoxyphenyl)-1,3,4-oxadiazol-2-amine

To a solution of 4-methoxybenzohydrazide (500 mg, 3.01 mmol, 1.00 eq) in methanol (3.00 mL) was added cyanic bromide (382 mg, 3.61 mmol, 266 uL, 1.20 eq). The mixture was stirred at 65° C. for 4 h. The reaction mixture was cooled to 25° C. Ethyl acetate (60 mL) and water (60 mL) were added into the reaction solution. The reaction solution was extracted with ethyl acetate (2×50 mL). The combined organic layers was washed with brine (50.0 mL), dried over sodium sulfate and filtered. The filtrate was concentrated under reduced pressure to give a residue. The residue was triturated with ethyl acetate (30.0 mL) at 25° C. for 15 min and filter. The filter cake was washed with saturated sodium bicarbonate solution and filtration to afford 5-(4-methoxyphenyl)-1,3,4-oxadiazol-2-amine (350 mg, 1.83 mmol, 60% yield) as a white solid.

¹H NMR (400 MHz, DMSO-d₆) δ=7.76-7.69 (m, 2H), 7.12 (s, 2H), 7.09-7.06 (m, 2H), 3.82 (s, 3H).

### Step 2. Procedure for Compound 3—2-bromo-5-(4-methoxyphenyl)-1,3,4-oxadiazole

To a solution of 5-(4-methoxyphenyl)-1,3,4-oxadiazol-2-amine (350 mg, 1.38 mmol, 1.00 eq) in acetonitrile (5.00 mL) was added cuprous bromide (397 mg, 2.76 mmol, 84.2 uL, 2.00 eq) and tert-butyl nitrite (285 mg, 2.76 mmol, 329 uL, 2.00 eq) at 0° C. The mixture was stirred at 60° C. for 3 h. The mixture was concentrated under reduced pressure to give a residue. The residue was purified by column chromatography (SiO₂, petroleum ether/ethyl acetate=4/1) to afford 2-bromo-5-(4-methoxyphenyl)-1,3,4-oxadiazole (220 mg, 750 umol, 54% yield, 87% purity) as a white solid.

¹H NMR (400 MHz, DMSO-d₆) δ=7.92 (d, J=8.6 Hz, 2H), 7.15 (d, J=8.8 Hz, 2H), 3.85 (s, 3H).

### Step 3. Procedure for 3-(2,6-difluoro-4-(3-((5-(4-methoxyphenyl)-1,3,4-oxadiazol-2-yl)amino)azetidin-1-yl)phenyl)piperidine-2,6-dione

To a solution of 3-(4-(3-aminoazetidin-1-yl)-2,6-difluorophenyl)piperidine-2,6-dione (160 mg, 542 umol, 1.00 eq), N,N-diisopropylethylamine (140 mg, 1.08 mmol, 189 uL, 2.00 eq) in dimethyl formamide (3.00 mL) was added 2-bromo-5-(4-methoxyphenyl)-1,3,4-oxadiazole (111 mg, 433 umol, 0.800 eq). The mixture was stirred at 85° C. for 12 h. The mixture was concentrated under reduced pressure to give a residue. The residue was purified by prep-HPLC (C18, 80 g; condition: water/acetonitrile=100:0 to 0:100, 0.1% formic acid) and lyophilized to afford 3-(2,6-difluoro-4-(3-((5-(4-methoxyphenyl)-1,3,4-oxadiazol-2-yl)amino)azetidin-1-yl)phenyl)piperidine-2,6-dione (51.19 mg, 107 umol, 19% yield, 99% purity) as a white solid.

¹H NMR (400 MHz, DMSO-d₆) δ=10.86 (s, 1H), 8.42 (br d, J=7.0 Hz, 1H), 7.75 (br d, J=8.6 Hz, 2H), 7.09 (br d, J=8.8 Hz, 2H), 6.21 (br d, J=11.2 Hz, 2H), 4.59-4.48 (m, 1H), 4.22 (br t, J=8.0 Hz, 2H), 4.05 (br dd, J=4.8, 12.2 Hz, 1H), 3.82 (s, 3H), 3.81-3.76 (m, 2H), 2.84-2.73 (m, 1H), 2.60-2.54 (m, 1H), 2.15-2.03 (m, 1H), 2.00-1.91 (m, 1H). MS (ESI) m/z. 470.1 [M+H]⁺

### Example 98. Synthesis of Compound 99

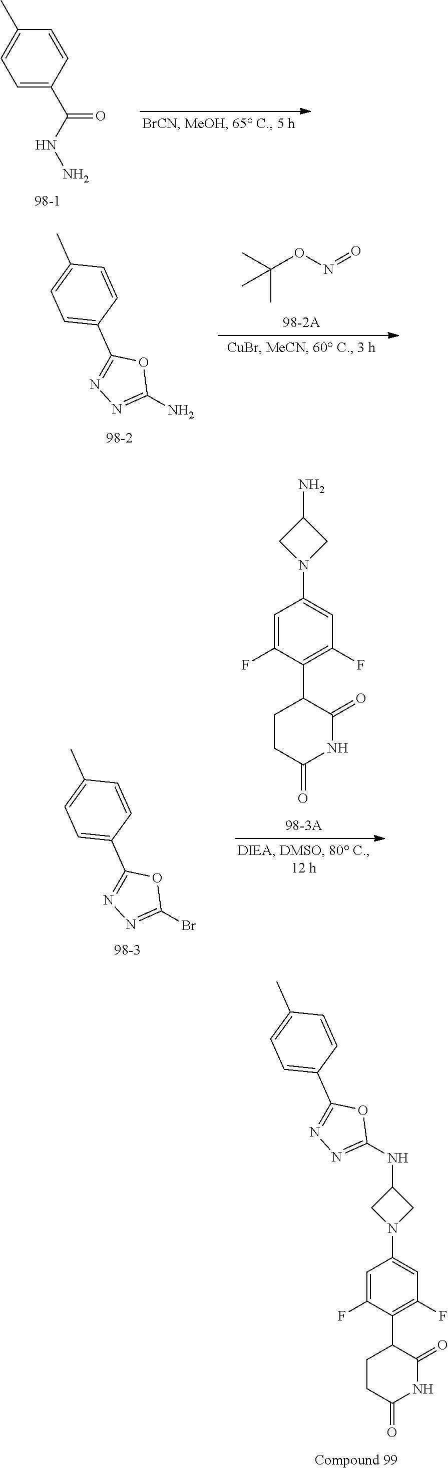

### Step 1. Procedure for Preparation of Compound 2—5-(p-tolyl)-1,3,4-oxadiazol-2-amine

To a solution of 4-methylbenzohydrazide (500 mg, 3.33 mmol, 1.00 eq) in methanol (5.00 mL) was added cyanic bromide (423 mg, 3.99 mmol, 293 uL, 1.20 eq). The mixture was stirred at 65° C. for 5 h. The reaction was concentrated under reduced pressure to give a residue. The residue was triturated with ethyl acetate (10.0 mL) and saturated sodium bicarbonate (10.0 mL) for 10 min to afford 5-(p-tolyl)-1,3,4-oxadiazol-2-amine (400 mg, 2.28 mmol, 69% yield) as a white solid.

¹H NMR (400 MHz, DMSO-d₆) δ=7.69 (d, J=8.0 Hz, 2H), 7.34 (d, J=8.0 Hz, 2H), 7.19 (s, 2H), 2.37 (s, 3H).

### Step 2. Procedure for Preparation of Compound 3—2-bromo-5-(p-tolyl)-1,3,4-oxadiazole

To a solution of 5-(p-tolyl)-1,3,4-oxadiazol-2-amine (410 mg, 2.34 mmol, 1.00 eq) in acetonitrile (6.00 mL) was added cuprous bromide (671 mg, 4.68 mmol, 142 uL, 2.00 eq) and tert-butyl nitrite (482 mg, 4.68 mmol, 556 uL, 2.00 eq). The reaction was stirred at 60° C. for 3 h under nitrogen atmosphere. The reaction was concentrated under reduced pressure to give a residue. The residue was purified by column chromatography (SiO₂, petroleum ether/ethyl acetate=10/1 to 8/1) to afford 2-bromo-5-(p-tolyl)-1,3,4-oxadiazole (200 mg, 836 umol, 36% yield) as a white solid.

¹H NMR (400 MHz, DMSO-d₆) δ=7.79 (d, J=8.0 Hz, 2H), 7.34 (d, J=8.0 Hz, 2H), 2.32 (s, 3H). MS (ESI) m/z 238.9 [M+H]⁺

### Step 3. Procedure for Preparation of 3-(2,6-difluoro-4-(3-((5-(p-tolyl)-1,3,4-oxadiazol-2-yl)amino)azetidin-1-yl)phenyl)piperidine-2,6-dione

To a solution of 2-bromo-5-(p-tolyl)-1,3,4-oxadiazole (100 mg, 418 umol, 1.00 eq) in dimethylsulfoxide (1.00 mL) was added N,N-diisopropylethylamine (162 mg, 1.25 mmol, 218 uL, 3.00 eq) and 3-(4-(3-aminoazetidin-1-yl)-2,6-difluorophenyl)piperidine-2,6-dione (185 mg, 627 umol, 1.50 eq). The reaction was stirred at 80° C. for 12 h. The reaction was filtered to give a filtrate. The filtrate was purified by Prep-HPLC (column: Phenomenex luna C18 150*25 mm*10 um; mobile phase: [water(formic acid)-acetonitrile]; B %: 35%-65%, 9 min) and lyophilized to afford 3-(2,6-difluoro-4-(3-((5-(p-tolyl)-1,3,4-oxadiazol-2-yl)amino)azetidin-1-yl)phenyl)piperidine-2,6-dione (67.57 mg, 147 umol, 35% yield, 99% purity) as a white solid.

¹H NMR (400 MHz, DMSO-d₆) δ=10.87 (s, 1H), 8.48 (d, J=6.8 Hz, 1H), 7.72 (d, J=8.0 Hz, 2H), 7.35 (d, J=7.6 Hz, 2H), 6.22 (d, J=11.1 Hz, 2H), 4.55 (br d, J=6.4 Hz, 1H), 4.23 (br t, J=7.6 Hz, 2H), 4.05 (br dd, J=5.2, 12.4 Hz, 1H), 3.82-3.78 (m, 2H), 2.79 (br s, 1H), 2.59 (br s, 1H), 2.37 (s, 3H), 2.12-2.04 (m, 1H), 2.01-1.93 (m, 1H). MS (ESI) m/z 454.2 [M+H]⁺

### Example 99. Synthesis of Compound 100

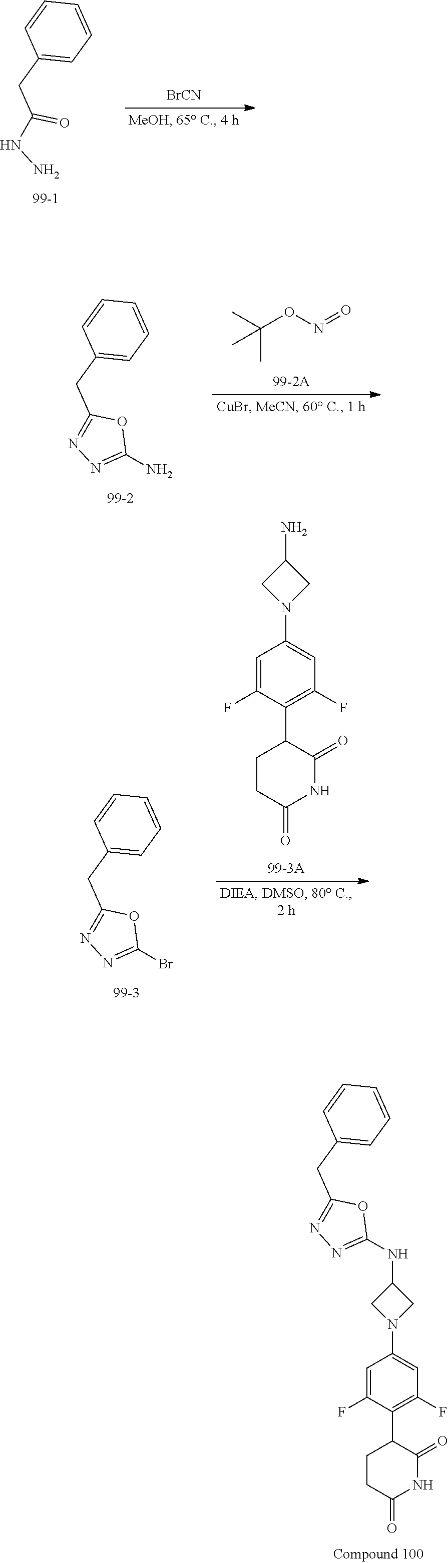

### Step 1. Procedure for Compound 2—5-benzyl-1,3,4-oxadiazol-2-amine

To a solution of 2-phenylacetohydrazide (712 mg, 4.74 mmol, 1.00 eq) in methanol (10.0 mL) was added carbononitridic bromide (603 mg, 5.70 mmol, 418 uL, 1.20 eq). The reaction mixture was stirred at 65° C. for 4 h. The reaction mixture was concentrated under reduced pressure. T The solid was triturated with ethyl acetate (50.0 mL) at 20° C. for 10 min to afford 5-benzyl-1,3,4-oxadiazol-2-amine (700 mg, 4.00 mmol, 84% yield) as a yellow solid.

¹H NMR (400 MHz, DMSO-d₆) δ=7.37-7.31 (m, 2H), 7.30-7.23 (m, 3H), 6.88 (br s, 2H), 4.02 (s, 2H).

### Step 2. Procedure for Compound 3—2-benzyl-5-bromo-1,3,4-oxadiazole

To a solution of 5-benzyl-1,3,4-oxadiazol-2-amine (350 mg, 2.00 mmol, 1.00 eq) in acetonitrile (5.00 mL) was added copper(II) bromide (892 mg, 4.00 mmol, 187 uL, 2.00 eq) and tert-butyl nitrite (412 mg, 4.00 mmol, 475 uL, 2 eq). The reaction mixture was stirred at 60° C. for 1 h. The reaction mixture was concentrated under reduced pressure. The residue was purified by column chromatography (SiO₂, petroleum ether/ethyl acetate=1/0 to 10/1) to afford 2-benzyl-5-bromo-1,3,4-oxadiazole (0.60 g, 1.66 mmol, 83% yield, 66% purity) as a yellow oil.

¹H NMR (400 MHz, DMSO-d₆) δ=7.39-7.28 (m, 5H), 4.31 (s, 2H)

### Step 3. Procedure for 3-(4-(3-((5-benzyl-1,3,4-oxadiazol-2-yl)amino)azetidin-1-yl)-2,6-difluorophenyl)piperidine-2,6-dione

To a solution of 2-benzyl-5-bromo-1,3,4-oxadiazole (48.9 mg, 204 umol, 0.8 eq) in dimethylsulfoxide (1.00 mL) was added 3-(4-(3-aminoazetidin-1-yl)-2,6-difluorophenyl)piperidine-2,6-dione (100 mg, 256 umol, 1.00 eq, methanesulfonic acid) and N,N-diisopropylethylamine (66.0 mg, 511 umol, 89.0 uL, 2.00 eq). The reaction mixture was stirred at 80° C. for 2 h. The reaction mixture was concentrated under reduced pressure. The residue was purified by Prep-HPLC (column: Phenomenex luna C18 150*25 mm*10 um; mobile phase: [water(formic acid)-acetonitrile]; B %: 31%-61%, 58 min) and lyophilized to afford 3-(4-(3-((5-benzyl-1,3,4-oxadiazol-2-yl)amino)azetidin-1-yl)-2,6-difluorophenyl)piperidine-2,6-dione (21.85 mg, 47.71 umol, 19% yield, 99% purity) as a off-white solid.

¹H NMR (400 MHz, DMSO-d₆) δ=10.86 (s, 1H), 8.16 (d, J=6.9 Hz, 1H), 7.39-7.32 (m, 2H), 7.31-7.24 (m, 3H), 6.17 (d, J=11.0 Hz, 2H), 4.47-4.37 (m, 1H), 4.16 (t, J=7.6 Hz, 2H), 4.08 (s, 2H), 4.04 (br dd, J=5.1, 12.6 Hz, 1H), 3.70 (dd, J=5.7, 7.6 Hz, 2H), 2.85-2.72 (m, 1H), 2.47 (br s, 1H), 2.07 (dq, J=3.6, 12.9 Hz, 1H), 1.99-1.85 (m, 1H).

MS (ESI) m/z. 454.3 [M+H]⁺

### Example 100. Synthesis of Compound 101

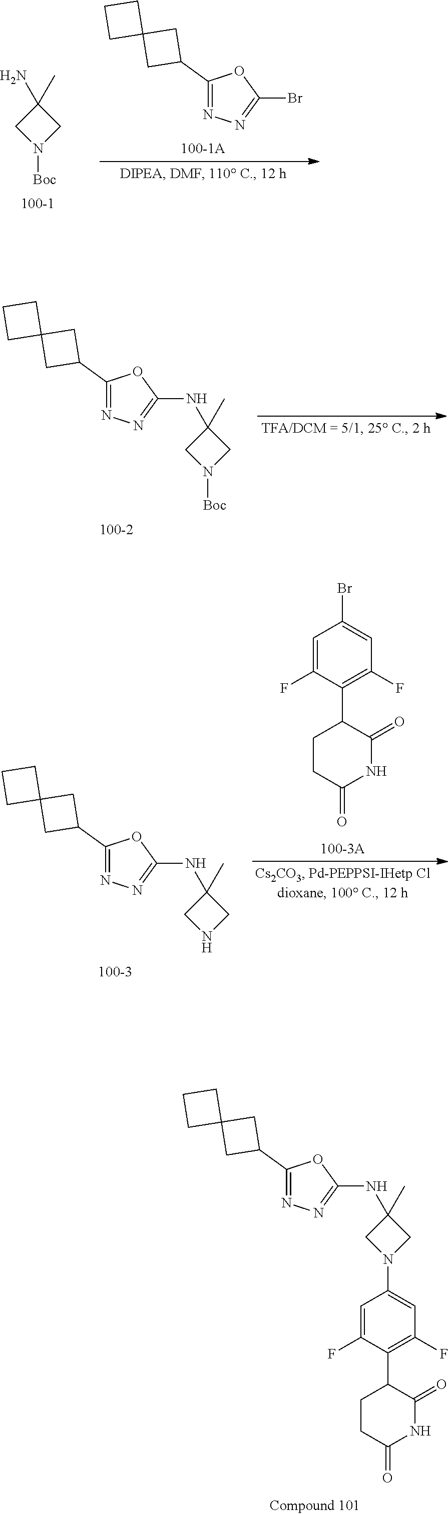

### Step 1. Procedure for Preparation of Compound 2—tert-butyl 3-methyl-3-((5-(spiro[3.3]heptan-2-yl)-1,3,4-oxadiazol-2-yl)amino)azetidine-1-carboxylate

To a solution of tert-butyl 3-amino-3-methylazetidine-1-carboxylate (63.8 mg, 342 umol, 1.00 eq) and 2-bromo-5-(spiro[3.3]heptan-2-yl)-1,3,4-oxadiazole (100 mg, 411 umol, 1.20 eq) in dimethylformamide (2.00 mL) was added N,N-diisopropylethylamine (132 mg, 1.03 mmol, 179 uL, 3.00 eq). The reaction was stirred at 100° C. for 12 h. The reaction was filtrated to give a filtrate. The filtrate was purified by reverse phase chromatography (C18, 40 g; condition: water/acetonitrile=100:0 to 0:100, 0.1% formic acid) and lyophilized to afford tert-butyl 3-methyl-3-((5-(spiro[3.3]heptan-2-yl)-1,3,4-oxadiazol-2-yl)amino)azetidine-1-carboxylate (70.0 mg, 200 umol, 59% yield) as a brown solid.

¹H NMR (400 MHz, CDCl₃-d) δ=5.23-4.92 (m, 1H), 4.12 (br d, J=8.4 Hz, 2H), 3.88 (d, J=8.8 Hz, 2H), 3.45 (t, J=8.4 Hz, 1H), 2.44-2.36 (m, 2H), 2.35-2.28 (m, 2H), 2.10 (t, J=7.2 Hz, 2H), 2.01-1.94 (m, 2H), 1.88-1.80 (m, 2H), 1.69 (s, 3H), 1.46 (s, 9H).

### Step 2. Procedure for Preparation of Compound 3—N-(3-methylazetidin-3-yl)-5-(spiro[3.3]heptan-2-yl)-1,3,4-oxadiazol-2-amine

A mixture of tert-butyl 3-methyl-3-((5-(spiro[3.3]heptan-2-yl)-1,3,4-oxadiazol-2-yl)amino)azetidine-1-carboxylate (70.0 mg, 200 umol, 1.00 eq) in dichloromethane (2.00 mL) and trifluoroacetic acid (400 uL) was stirred at 25° C. for 2 h. The reaction was concentrated under reduced pressure to afford N-(3-methylazetidin-3-yl)-5-(spiro[3.3]heptan-2-yl)-1,3,4-oxadiazol-2-amine (45.0 mg, 181 umol, 90% yield) as colorless oil. MS (ESI) m/z 249.0 [M+H]⁺

### Step 3. Procedure for Preparation of 3-(2,6-difluoro-4-(3-methyl-3-((5-(spiro[3.3]heptan-2-yl)-1,3,4-oxadiazol-2-yl)amino)azetidin-1-yl)phenyl)piperidine-2,6-dione

To a solution of 3-(4-bromo-2,6-difluorophenyl)piperidine-2,6-dione (66.1 mg, 217 umol, 1.20 eq) and N-(3-methylazetidin-3-yl)-5-(spiro[3.3]heptan-2-yl)-1,3,4-oxadiazol-2-amine (45.0 mg, 181 umol, 1.00 eq) in dioxane (1.00 mL) were added cesium carbonate (177 mg, 543 umol, 3.00 eq) and [1,3-bis[2,6-bis(1-propylbutyl)phenyl]-4,5-dichloro-imidazol-2-ylidene]-dichloro-(3-chloropyridin-1-ium-1-yl)palladium (3.53 mg, 3.62 umol, 0.020 eq). The reaction was stirred at 100° C. for 12 h under nitrogen atmosphere. The reaction mixture was filtered. The filtrate was purified by reverse phase chromatography (C18, 40 g; condition: water/acetonitrile=100:0 to 0:100, 0.1% formic acid) to give a crude product. The crude product was purified by Prep-HPLC (column: Phenomenex luna C18 150*25 mm*10 um; mobile phase: [water (formic acid)-acetonitrile]; B %: 43%-73%, 9 min) and lyophilized to afford 3-(2,6-difluoro-4-(3-methyl-3-((5-(spiro[3.3]heptan-2-yl)-1,3,4-oxadiazol-2-yl)amino)azetidin-1-yl)phenyl)piperidine-2,6-dione (11.46 mg, 23.3 umol, 13% yield, 96% purity) as a white solid.

¹H NMR (400 MHz, DMSO-d₆) δ=10.86 (s, 1H), 8.03 (s, 1H), 6.18 (d, J=11.2 Hz, 2H), 4.04 (br dd, J=4.8, 12.6 Hz, 1H), 3.97 (d, J=7.6 Hz, 2H), 3.78 (d, J=7.6 Hz, 2H), 3.42 (br s, 1H), 2.81-2.73 (m, 1H), 2.49-2.46 (m, 1H), 2.37-2.31 (m, 2H), 2.22-2.16 (m, 2H), 2.12-2.07 (m, 1H), 2.07-2.02 (m, 2H), 1.99-1.93 (m, 1H), 1.93-1.88 (m, 2H), 1.82-1.73 (m, 2H), 1.61 (s, 3H). MS (ESI) m/z 472.2 [M+H]⁺

### Example 101. Synthesis of Compound 102

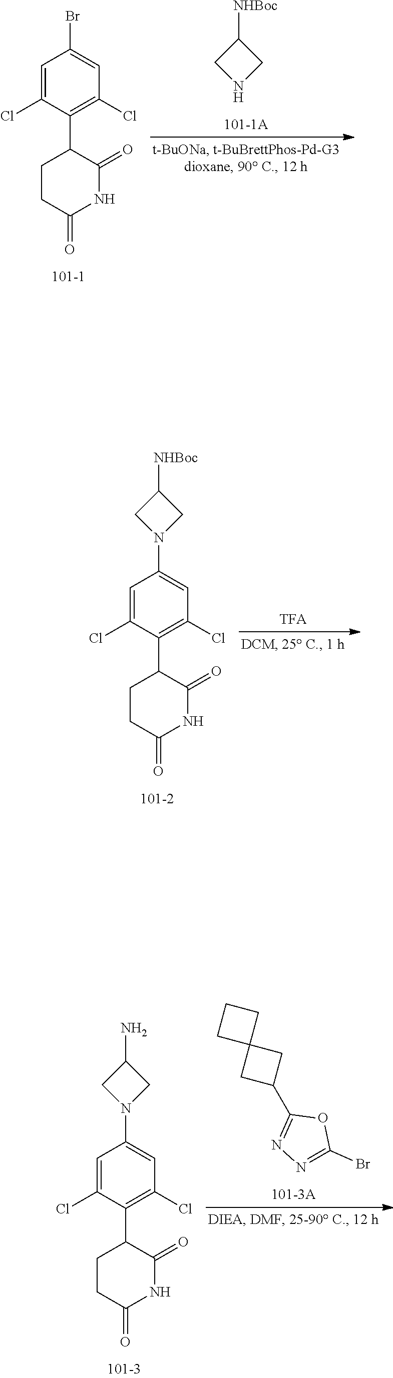 [SMILES: O=C1CCC(c2c(Cl)cc(Br)cc2Cl)C(=O)N1 ; CC(C)(C)OC(=O)NC1CNC1 ; CC(C)(C)OC(=O)NC1CN(c2cc(Cl)c(C3CCC(=O)NC3=O)c(Cl)c2)C1 ; NC1CN(c2cc(Cl)c(C3CCC(=O)NC3=O)c(Cl)c2)C1 ; Brc1nnc(C2CC3(CCC3)C2)o1]

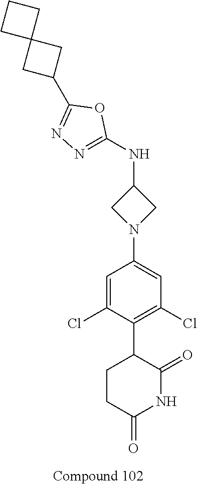 [SMILES: O=C1CCC(c2c(Cl)cc(N3CC(Nc4nnc(C5CC6(CCC6)C5)o4)C3)cc2Cl)C(=O)N1]

### Step 1. Procedure for Compound 1—tert-butyl (1-(3,5-dichloro-4-(2,6-dioxopiperidin-3-yl)phenyl)azetidin-3-yl)carbamate

A mixture of 3-(4-bromo-2,6-dichlorophenyl)piperidine-2,6-dione (300 mg, 890 umol, 1.00 eq), tert-butyl azetidin-3-ylcarbamate (307 mg, 1.78 mmol, 2.00 eq), sodium tert-butoxide (171 mg, 1.78 mmol, 2.00 eq), methanesulfonato[2-(di-tert-butylphosphino)-3,6-dimethoxy-2′,4′,6′-tri-i-propyl-1,1′-biphenyl](2′-amino-1,1′-biphenyl-2-yl)palladium(II) (76.1 mg, 89.0 umol, 0.100 eq) in dioxane (5.00 mL) was degassed and purged with nitrogen for 3 times. The reaction mixture was stirred at 90° C. for 12 h under nitrogen atmosphere. The resulting mixture was filtered over Celite and the filter was added into water (50.0 mL) and extracted with ethyl acetate (3×50.0 mL). The combined organic layers were washed with brine (50.0 mL), dried over sodium sulfate and filtered. The filtrate was concentrated under reduced pressure to give a residue. The residue was purified by prep-HPLC (C18, 80 g; condition: water/acetonitrile=100:0 to 0:100, 0.1% formic acid) and lyophilized to afford tert-butyl (1-(3,5-dichloro-4-(2,6-dioxopiperidin-3-yl)phenyl)azetidin-3-yl)carbamate (400 mg, 925 umol, 20% yield, 99% purity) was obtained as a white solid.

¹H NMR (400 MHz, DMSO-d₆) δ=10.86 (s, 1H), 7.55 (br d, J=7.6 Hz, 1H), 6.49 (dd, J=2.2, 14.2 Hz, 2H), 4.40 (dd, J=5.6, 12.6 Hz, 2H), 4.10 (t, J=7.6 Hz, 2H), 3.62 (t, J=6.8 Hz, 2H), 2.82 (ddd, J=6.0, 14.3, 16.9 Hz, 1H), 2.52 (br s, 1H), 2.35-2.23 (m, 1H), 1.90-1.79 (m, 1H), 1.39 (s, 9H).

### Step 2. Procedure for Compound 3—3-(4-(3-aminoazetidin-1-yl)-2,6-dichlorophenyl)piperidine-2,6-dione

To a solution of tert-butyl (1-(3,5-dichloro-4-(2,6-dioxopiperidin-3-yl)phenyl)azetidin-3-yl)carbamate (200 mg, 467 umol, 1.00 eq) in dichloromethane (5.00 mL) was added trifluoroacetic acid (1.00 mL). The mixture was stirred at 25° C. for 1 h. The mixture was concentrated under reduced pressure to afford 3-(4-(3-aminoazetidin-1-yl)-2,6-dichlorophenyl)piperidine-2,6-dione (150 mg, crude) as yellow oil. MS (ESI) m/z. 328.0 [M+H]⁺

### Step 3. Procedure for 3-(2,6-dichloro-4-(3-((5-(spiro[3.3]heptan-2-yl)-1,3,4-oxadiazol-2-yl)amino)azetidin-1-yl)phenyl)piperidine-2,6-dione

To a solution of 3-(4-(3-aminoazetidin-1-yl)-2,6-dichlorophenyl)piperidine-2,6-dione (150 mg, 457 umol, 1.00 eq) in dimethylsulfoxide (2.00 mL) was added N,N-diisopropylethylamine (118 mg, 914 umol, 159.22 uL, 2.00 eq) and 2-bromo-5-(spiro[3.3]heptan-2-yl)-1,3,4-oxadiazole (88.9 mg, 366 umol, 0.800 eq) at 25° C. The mixture was stirred at 90° C. for 12 h. The mixture was concentrated under reduced pressure to give a residue. The residue was purified by prep-HPLC (C18, 80 g; condition: water/acetonitrile=100:0 to 0:100, 0.1% formic acid) and lyophilized to afford the crude product. The crude product was further purified by column chromatography (SiO₂, petroleum ether/ethyl acetate=100/1) to afford 3-(2,6-dichloro-4-(3-((5-(spiro[3.3]heptan-2-yl)-1,3,4-oxadiazol-2-yl)amino)azetidin-1-yl)phenyl)piperidine-2,6-dione (11.47 mg, 23.2 umol, 5% yield, 99% purity) as a white solid.

¹H NMR (400 MHz, DMSO-d₆) δ=10.95-10.74 (m, 1H), 8.12 (br d, J=7.0 Hz, 1H), 6.56 (d, J=1.4 Hz, 1H), 6.53 (s, 1H), 4.47-4.36 (m, 2H), 4.20 (br t, J=7.8 Hz, 2H), 3.78-3.70 (m, 2H), 3.42-3.37 (m, 1H), 2.87-2.78 (m, 1H), 2.46-2.46 (m, 1H), 2.38 (br s, 1H), 2.34 (br d, J=7.0 Hz, 2H), 2.23-2.17 (m, 2H), 2.05 (br t, J=7.2 Hz, 2H), 1.90 (q, J=7.0 Hz, 2H), 1.87-1.81 (m, 1H), 1.81-1.74 (m, 2H). MS (ESI) m/z. 490.1 [M+H]⁺

### Example 102. Synthesis of Compound 103

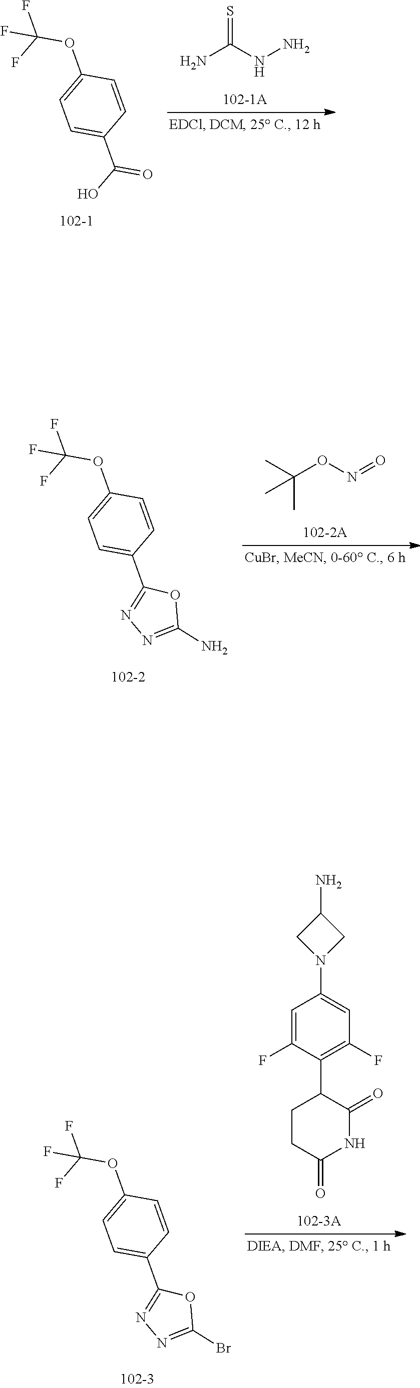

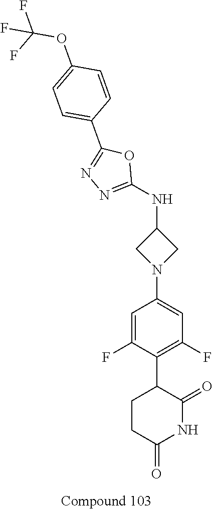 [SMILES: O=C1CCC(c2c(F)cc(N3CC(Nc4nnc(-c5ccc(OC(F)(F)F)cc5)o4)C3)cc2F)C(=O)N1]

### Step 1. Procedure for Compound 2—5-(4-(trifluoromethoxy)phenyl)-1,3,4-oxadiazol-2-amine

To a solution of 4-(trifluoromethoxy)benzoic acid (1.00 g, 4.85 mmol, 1.00 eq), hydrazinecarbothioamide (663 mg, 7.28 mmol, 1.5 eq) in dichloromethane (10.0 mL) was added 1-(3-dimethylaminopropyl)-3-ethylcarbodiimide hydrochloride (2.79 g, 14.6 mmol, 3.00 eq). The mixture was stirred at 25° C. for 12 h. The residue was diluted with water (50 mL) and extracted with dichloromethane (3×50 mL). The combined organic layers were dried over anhydrous sodium sulfate and filtered. The filtrate was concentrated under reduced pressure to give a residue. The residue was purified by column chromatography (SiO₂, petroleum ether/ethyl acetate=5/1) to give 5-(4-(trifluoromethoxy)phenyl)-1,3,4-oxadiazol-2-amine (1.10 g, 4.49 mmol, 30% yield) as a white solid.

¹H NMR (400 MHz, DMSO-d₆) δ=7.93-7.87 (m, 2H), 7.53 (d, J=8.0 Hz, 2H), 7.34 (s, 2H).

### Step 2. Procedure for Compound 3—2-bromo-5-(4-(trifluoromethoxy)phenyl)-1,3,4-oxadiazole

To a solution of 5-(4-(trifluoromethoxy)phenyl)-1,3,4-oxadiazol-2-amine (500 mg, 2.04 mmol, 1.00 eq) in acetonitrile (5.00 mL) was added cuprous bromide (585 mg, 4.08 mmol, 124 uL, 2.00 eq) and tert-butyl nitrite (421 mg, 4.08 mmol, 485 uL, 2.00 eq) at 0° C. The mixture was stirred at 60° C. for 6 h. The mixture was concentrated under reduced pressure to give a residue. The residue was concentrated under reduced pressure to give a residue. The residue was purified by column chromatography (SiO₂, petroleum ether/ethyl acetate=9/1) to give 2-bromo-5-(4-(trifluoromethoxy)phenyl)-1,3,4-oxadiazole (350 mg, 1.12 mmol, 54% yield, 99% purity) as a white solid.

¹H NMR (400 MHz, DMSO-d₆) δ=8.10 (dd, J=2.0, 8.9 Hz, 2H), 7.60 (br dd, J=1.0, 7.9 Hz, 2H).

### Step 3. Procedure for 3-(2,6-difluoro-4-(3-((5-(4-(trifluoromethoxy)phenyl)-1,3,4-oxadiazol-2-yl)amino)azetidin-1-yl)phenyl)piperidine-2,6-dione

A solution of 3-(4-(3-aminoazetidin-1-yl)-2,6-difluorophenyl)piperidine-2,6-dione (90.0 mg, 305 umol, 1.00 eq), N,N-diisopropylethylamine (78.8 mg, 610 umol, 106 uL, 2.00 eq) in dimethyl formamide (2.00 mL) was added 2-bromo-5-(4-(trifluoromethoxy)phenyl)-1,3,4-oxadiazole (104 mg, 335 umol, 1.10 eq). The mixture was stirred at 25° C. for 1 h. The mixture was concentrated under reduced pressure to give a residue. The residue was purified by column chromatography (SiO₂, petroleum ether/ethyl acetate=0/1) and lyophilized to afford 3-(2,6-difluoro-4-(3-((5-(4-(trifluoromethoxy)phenyl)-1,3,4-oxadiazol-2-yl) amino)azetidin-1-yl)phenyl)piperidine-2,6-dione (63.42 mg, 120 umol, 39% yield, 99% purity) as a white solid.

¹H NMR (400 MHz, DMSO-d₆) δ=10.87 (s, 1H), 8.62 (d, J=6.8 Hz, 1H), 7.95 (d, J=8.8 Hz, 2H), 7.55 (br d, J=8.4 Hz, 2H), 6.22 (d, J=11.0 Hz, 2H), 4.64-4.51 (m, 1H), 4.23 (t, J=7.6 Hz, 2H), 4.11-4.01 (m, 1H), 3.85-3.77 (m, 2H), 2.84-2.73 (m, 1H), 2.55-2.53 (m, 1H), 2.15-2.04 (m, 1H), 2.01-1.91 (m, 1H). MS (ESI) m/z. 524.2 [M+H]⁺

### Example 103. Synthesis of Compound 104

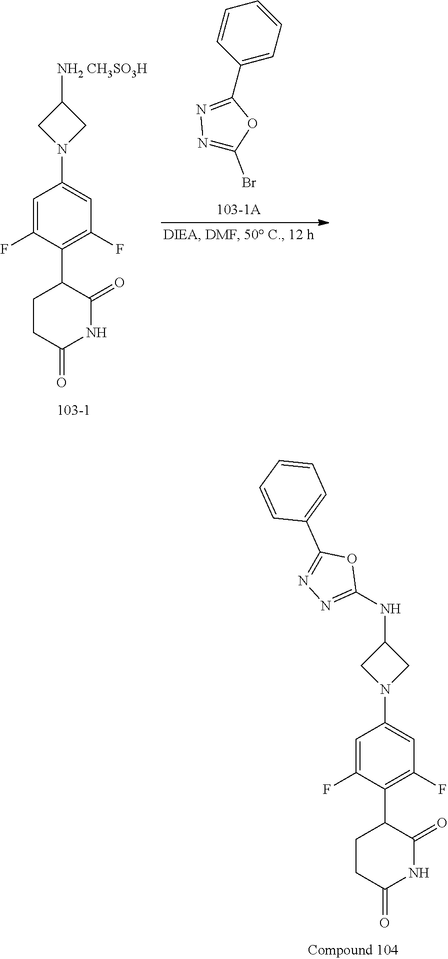

### Step 1. Procedure for 3-(2,6-difluoro-4-(3-((5-phenyl-1,3,4-oxadiazol-2-yl)amino)azetidin-1-yl)phenyl)piperidine-2,6-dione

To a solution of 3-(4-(3-aminoazetidin-1-yl)-2,6-difluorophenyl)piperidine-2,6-dione (50.0 mg, 128 umol, 1.00 eq, mesylate) in dimethylformamide (1.00 mL) were added N,N-diisopropylethylamine (65.7 mg, 508 umol, 88.5 uL, 3.00 eq) and 2-bromo-5-phenyl-1,3,4-oxadiazole (45.7 mg, 203 umol, 1.20 eq). The mixture was stirred at 50° C. for 12 h under nitrogen atmosphere. The mixture was filtered. The filtrate was purified by Prep-HPLC (column: Phenomenex luna C18 150*25 mm*10 um; mobile phase: [water (formic acid)-acetonitrile]; B %: 27%-57%, min) and lyophilized to afford 3-(2,6-difluoro-4-(3-((5-phenyl-1,3,4-oxadiazol-2-yl)amino)azetidin-1-yl)phenyl)piperidine-2,6-dione (13.16 mg, 29.65 umol, 17% yield, 99% purity) as a white solid.

¹H NMR (400 MHz, DMSO-d₆) δ=10.86 (s, 1H), 8.53 (d, J=6.9 Hz, 1H), 7.86-7.79 (m, 2H), 7.58-7.50 (m, 3H), 6.21 (d, J=11.0 Hz, 2H), 4.62-4.50 (m, 1H), 4.23 (t, J=7.7 Hz, 2H), 4.11-3.99 (m, 1H), 3.86-3.74 (m, 2H), 2.85-2.72 (m, 1H), 2.55-2.52 (m, 1H), 2.16-2.02 (m, 1H), 2.00-1.89 (m, 1H). MS (ESI) m/z 440.0 [M+H]⁺

### Example 104. Synthesis of Compound 105

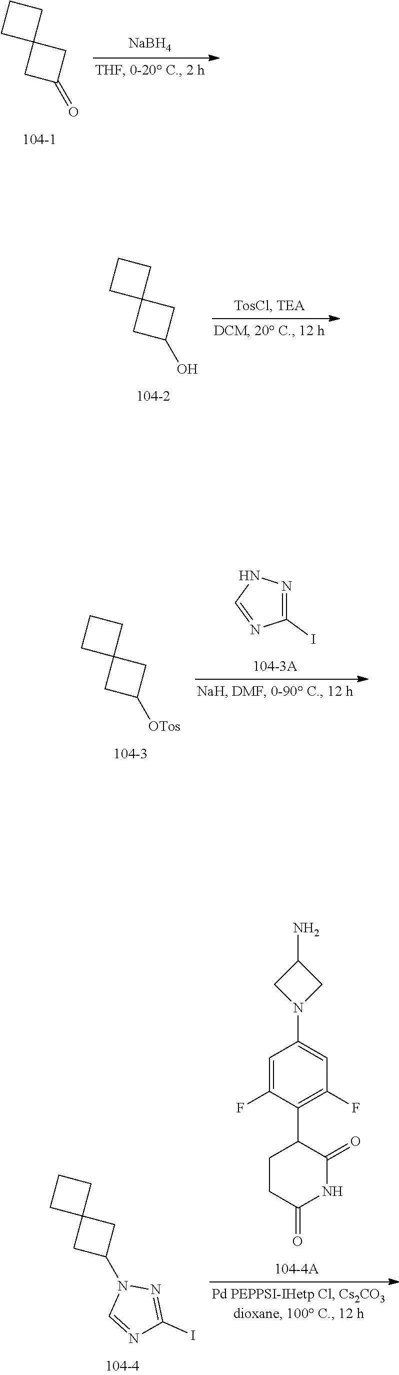

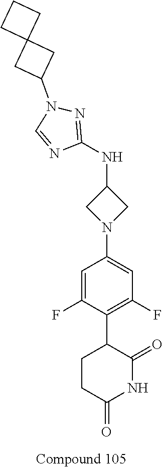 [SMILES: O=C1CCC(c2c(F)cc(N3CC(Nc4ncn(C5CC6(CCC6)C5)n4)C3)cc2F)C(=O)N1]

### Step 1. Procedure for Preparation of Compound 2—spiro[3.3]heptan-2-ol

To a solution of spiro[3.3]heptan-2-one (1.00 g, 9.08 mmol, 1.00 eq) in tetrahydrofuran (10.0 mL) was added sodium borohydride (687 mg, 18.2 mmol, 2.00 eq) at 0° C. The reaction mixture was stirred at 20° C. for 2 h. The reaction mixture was quenched with saturated ammonium chloride (30 mL) and then extracted with ethyl acetate (3×20 mL). The organic layers were washed with brine (40 mL), dried over anhydrous sodium sulfate, filtered and concentrated under reduced pressure to afford spiro[3.3]heptan-2-ol (1.00 g, 8.92 mmol, 98% yield) as colorless oil.

¹H NMR (400 MHz, CDCl₃-d) δ=4.20-4.09 (m, 1H), 2.43-2.36 (m, 2H), 1.95 (q, J=7.5 Hz, 4H), 1.86-1.80 (m, 4H).

### Step 2. Procedure for Preparation of Compound 3—spiro[3.3]heptan-2-yl 4-methylbenzenesulfonate

To a solution of spiro[3.3]heptan-2-ol (500 mg, 4.46 mmol, 1.00 eq) in dichloromethane (5.00 mL) was added triethylamine (902 mg, 8.92 mmol, 1.24 mL, 2.00 eq) and 4-methylbenzene-1-sulfonyl chloride (935 mg, 4.90 mmol, 1.10 eq). The reaction mixture was stirred at 20° C. for 12 h. The reaction mixture was added water (10 mL) and then extracted with dichloromethane (3×8 mL). The combined organic layers were washed with saturated sodium bicarbonate (10 mL), dried over anhydrous sodium sulfate, filtered and concentrated under reduced pressure to give a residue. The residue was purified by column chromatography (SiO₂, petroleum ether/ethyl acetate=1/0 to 10/1) to afford spiro[3.3]heptan-2-yl 4-methylbenzenesulfonate (320 mg, 1.20 mmol, 27% yield) as yellow oil.

¹H NMR (400 MHz, DMSO-d₆) δ=7.78 (d, J=8.3 Hz, 2H), 7.34 (d, J=8.0 Hz, 2H), 4.68 (quin, J=7.4 Hz, 1H), 2.46 (s, 3H), 2.33-2.27 (m, 2H), 2.07 (qdd, J=2.5, 7.4, 9.9 Hz, 2H), 1.96-1.91 (m, 4H), 1.84-1.74 (m, 2H).

### Step 3. Procedure for Preparation of Compound 4—3-iodo-1-(spiro[3.3]heptan-2-yl)-1H-1,2,4-triazole

To a solution of 3-iodo-1H-1,2,4-triazole (154 mg, 788 umol, 1.00 eq) in dimethylformamide (2.00 mL) were added sodium hydride (47.3 mg, 1.18 mmol, 60% purity, 1.50 eq) at 0° C. The mixture was stirred at 20° C. for 0.5 h. Then spiro[3.3]heptan-2-yl 4-methylbenzenesulfonate (210 mg, 788 umol, 1.00 eq) in dimethylformamide (0.500 mL) was added to the mixture and the reaction mixture was stirred at 90° C. for 11.5 h. The reaction mixture was added to water (10 mL) to quench and then extracted with ethyl acetate (3×8 mL). The combined organic layers were dried over anhydrous sodium sulfate, filtered and concentrated under reduced pressure to give a residue. The residue was purified by reverse phase chromatography (C18, 40 g; condition: water/acetonitrile=100:0 to 34:66, 0.1% formic acid) and lyophilized to afford 3-iodo-1-(spiro[3.3]heptan-2-yl)-1H-1,2,4-triazole (106 mg, 367 umol, 47% yield) as yellow oil.

¹H NMR (400 MHz, CDCl₃-d) δ=7.97-7.87 (m, 1H), 4.81-4.60 (m, 1H), 2.61-2.47 (m, 4H), 2.16-2.09 (m, 2H), 2.07-2.02 (m, 2H), 1.94-1.85 (m, 2H).

### Step 4. Procedure for Preparation of 3-(2,6-difluoro-4-(3-((1-(spiro[3.3]heptan-2-yl)-1H-1,2,4-triazol-3-yl)amino)azetidin-1-yl)phenyl)piperidine-2,6-dione

To a solution of 3-(4-(3-aminoazetidin-1-yl)-2,6-difluorophenyl)piperidine-2,6-dione (135 mg, 346 umol, 1.00 eq, mesylate) in dioxane (2.00 mL) were added [1,3-bis[2,6-bis(1-propylbutyl)phenyl]-4,5-dichloro-imidazol-2-ylidene]-dichloro-(3-chloropyridin-1-ium-1-yl)palladium (33.7 mg, 34.6 umol, 0.100 eq), cesium carbonate (338 mg, 1.04 mmol, 3.00 eq) and 3-iodo-1-(spiro[3.3]heptan-2-yl)-1H-1,2,4-triazole (100 mg, 346 umol, 1.00 eq). The reaction mixture was stirred at 100° C. for 12 h under nitrogen atmosphere. The reaction mixture was concentrated under reduced pressure to give a residue. The residue was diluted with dimethylformamide (1 mL) and then filtered. The filtrate was purified by Prep-HPLC (column: Phenomenex luna C18 150*25 mm*10 um; mobile phase: [water (formic acid)-acetonitrile]; B %: 38%-68%, 9 min) and lyophilized to afford 3-(2,6-difluoro-4-(3-((1-(spiro[3.3]heptan-2-yl)-1H-1,2,4-triazol-3-yl)amino)azetidin-1-yl)phenyl)piperidine-2,6-dione (40.31 mg, 86.54 umol, 25% yield, 98% purity) as an off-white solid.

¹H NMR (400 MHz, DMSO-d₆) δ=10.85 (s, 1H), 8.06 (s, 1H), 6.59 (d, J=7.6 Hz, 1H), 6.15 (s, 1H), 6.12 (s, 1H), 4.57 (quin, J=8.0 Hz, 1H), 4.43 (qd, J=6.6, 13.1 Hz, 1H), 4.12 (t, J=7.4 Hz, 2H), 4.02 (br dd, J=4.9, 12.4 Hz, 1H), 3.64 (br t, J=6.6 Hz, 2H), 2.83-2.72 (m, 1H), 2.47 (br s, 1H), 2.44-2.31 (m, 4H), 2.13-2.01 (m, 3H), 2.00-1.89 (m, 3H), 1.87-1.75 (m, 2H). MS (ESI) m/z 457.2 [M+H]⁺

### Example 105. Synthesis of Compound 106

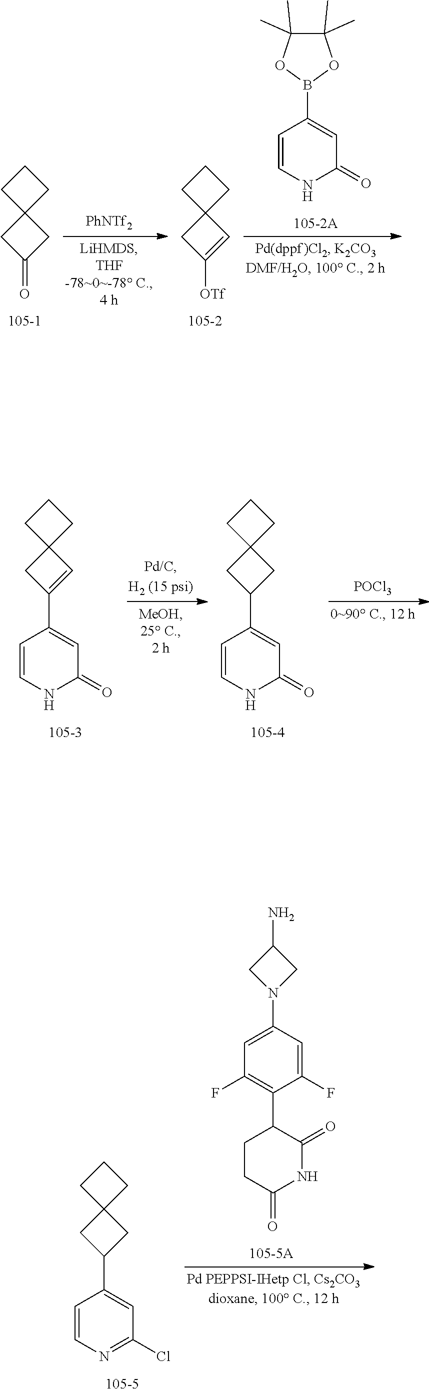

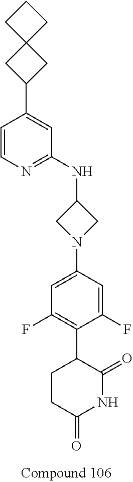 [SMILES: O=C1CCC(c2c(F)cc(N3CC(Nc4cc(C5CC6(CCC6)C5)ccn4)C3)cc2F)C(=O)N1]

### Step 1. Procedure for Preparation of Compound 2—spiro[3.3]hept-1-en-2-yl trifluoromethanesulfonate

To a solution of spiro[3.3]heptan-2-one (1.00 g, 9.08 mmol, 1.00 eq) in tetrahydrofuran (10.0 mL) was added dropwise to lithium bis(trimethylsilyl)amide (1.00 M, 11.8 mL, 1.30 eq) at −78° C. under nitrogen atmosphere. The solution was stirred at 0° C. for 2 h. After cooling to −78° C., a solution of 1,1,1-trifluoro-N-phenyl-N-((trifluoromethyl)sulfonyl)methanesulfonamide (3.57 g, 9.99 mmol, 1.10 eq) in tetrahydrofuran (5.00 mL) was added dropwise to the mixture. The mixture was stirred at −78° C. for 2 h. The reaction mixture was quenched by addition saturated ammonium chloride (10 mL) at 0° C. and extracted with ethyl acetate (3×20 mL). The combined organic layers were dried over sodium sulfate, filtered and concentrated under reduced pressure to give a residue. The residue was purified by column chromatography (SiO₂, petroleum ether) to afford spiro[3.3]hept-1-en-2-yl trifluoromethanesulfonate (1.27 g, 5.24 mmol, 58% yield) as colorless oil.

¹H NMR (400 MHz, CDCl₃-d) δ=5.45 (s, 1H), 2.80 (s, 2H), 2.15-2.05 (m, 4H), 1.87-1.75 (m, 2H).

### Step 2. Procedure for Preparation of Compound 3—4-(spiro[3.3]hept-1-en-2-yl)pyridin-2(1H)-one

A mixture of spiro[3.3]hept-1-en-2-yl trifluoromethanesulfonate (500 mg, 2.06 mmol, 1.00 eq), 4-(4,4,5,5-tetramethyl-1,3,2-dioxaborolan-2-yl)pyridin-2(1H)-one (548 mg, 2.48 mmol, 1.20 eq), potassium carbonate (571 mg, 4.13 mmol, 2.00 eq), [1,1-bis(diphenylphosphino)ferrocene]dichloropalladium(II) (169 mg, 206 umol, 0.100 eq) in dimethylformamide (8.00 mL) and water (2.00 mL) was degassed and purged with nitrogen atmosphere for 3 times. The mixture was stirred at 100° C. for 2 h under nitrogen atmosphere. The reaction mixture was filtered and the filtrate was concentrated under reduced pressure to give a residue. The residue was purified by reverse phase chromatography (C18, 40 g; condition: water/acetonitrile=100:0 to 0:100, 0.1% formic acid) and lyophilized to afford (spiro[3.3]hept-1-en-2-yl)pyridin-2(1H)-one (120 mg, 641 umol, 31% yield) as a gray solid. MS (ESI) m/z 188.0 [M+H]⁺

### Step 3. Procedure for Preparation of Compound 4—4-(spiro[3.3]heptan-2-yl)pyridin-2(1H)-one

To a solution of (spiro[3.3]hept-1-en-2-yl)pyridin-2(1H)-one (120 mg, 641 umol, 1.00 eq) in methanol (2.00 mL) was added palladium on activated carbon (20.0 mg, 10% purity) was degassed and purged with hydrogen atmosphere for 3 times. The mixture was stirred at 25° C. for 2 h under 15 psi of hydrogen atmosphere. The reaction mixture was filtered and the filtrate was concentrated under reduced pressure to give a residue. The residue was purified by reverse phase chromatography (C18, 40 g; condition: water/acetonitrile=100:0 to 0:100, 0.1% formic acid) and lyophilized to afford 4-(spiro[3.3]heptan-2-yl)pyridin-2(1H)-one (120 mg, 634 umol, 99% yield) as a white solid.

¹H NMR (400 MHz, DMSO-d₆) δ=11.47-11.24 (m, 1H), 7.29 (d, J=6.6 Hz, 1H), 6.23 (dd, J=1.6, 6.8 Hz, 1H), 6.09-6.03 (m, 1H), 2.67 (s, 2H), 2.21-2.10 (m, 4H), 1.95-1.86 (m, 2H). MS (ESI) m/z 190.0 [M+H]⁺

### Step 4. Procedure for Preparation of Compound 5—2-chloro-4-(spiro[3.3]heptan-2-yl)pyridine

4-(spiro[3.3]heptan-2-yl)pyridin-2(1H)-one (120 mg, 634 umol, 1.00 eq) was dissolved in phosphorus oxychloride (16.5 g, 108 mmol, 10.0 mL, 170 eq) at 0° C. and the mixture was stirred at 90° C. for 12 h. The reaction mixture was poured into ice water (10 mL) and extracted with ethyl acetate (3×10 mL). The combined organic layers were dried over sodium sulfate, filtered and concentrated under reduced pressure to give a residue. The residue was purified by reverse phase chromatography (C18, 40 g; condition: water/acetonitrile=100:0 to 0:100, 0.1% formic acid) and lyophilized to afford 2-chloro-4-(spiro[3.3]heptan-2-yl)pyridine (100 mg, 477 umol, 75% yield, 99% purity) as a brown solid. MS (ESI) m/z 207.9 [M+H]⁺

### Step 5. Procedure for Preparation of 3-(2,6-difluoro-4-(3-((4-(spiro[3.3]heptan-2-yl)pyridin-2-yl)amino)azetidin-1-yl)phenyl)piperidine-2,6-dione

A mixture of 3-(4-(3-aminoazetidin-1-yl)-2,6-difluorophenyl)piperidine-2,6-dione (70.0 mg, 179 umol, 1.00 eq, mesylate), 2-chloro-4-(spiro[3.3]heptan-2-yl)pyridine (40.9 mg, 197 umol, 1.10 eq), cesium carbonate (175 mg, 537 umol, 3.00 eq) and [1,3-bis[2,6-bis(1-propylbutyl)phenyl]-4,5-dichloro-imidazol-2-ylidene]-dichloro-(3-chloropyridin-1-ium-1-yl)palladium (17.4 mg, 17.9 umol, 0.100 eq) in dioxane (3.00 mL) was degassed and purged with nitrogen atmosphere for 3 times. The mixture was stirred at 100° C. for 12 h under nitrogen atmosphere. The reaction mixture was filtered and concentrated under reduced pressure to give a residue. The residue was purified by reverse phase chromatography (C18, 40 g; condition: water/acetonitrile=100:0 to 0:100) and lyophilized to afford 3-(2,6-difluoro-4-(3-((4-(spiro[3.3]heptan-2-yl)pyridin-2-yl)amino)azetidin-1-yl)phenyl)piperidine-2,6-dione (12.81 mg, 26.91 umol, 15% yield, 98% purity) as a white solid.

¹H NMR (400 MHz, DMSO-d₆) δ=10.86 (s, 1H), 7.88 (d, J=5.4 Hz, 1H), 7.06 (d, J=6.5 Hz, 1H), 6.40 (dd, J=1.1, 5.3 Hz, 1H), 6.30 (s, 1H), 6.16 (d, J=11.1 Hz, 2H), 4.75-4.57 (m, 1H), 4.18 (t, J=7.5 Hz, 2H), 4.04 (dd, J=5.3, 12.6 Hz, 1H), 3.66-3.58 (m, 2H), 3.20 (t, J=8.8 Hz, 1H), 2.86-2.73 (m, 1H), 2.55-2.52 (m, 1H), 2.39-2.27 (m, 2H), 2.15-2.04 (m, 3H), 2.01-1.93 (m, 3H), 1.91-1.86 (m, 2H), 1.84-1.76 (m, 2H). MS (ESI) m/z 467.3 [M+H]⁺

### Example 106. Synthesis of Compound 107

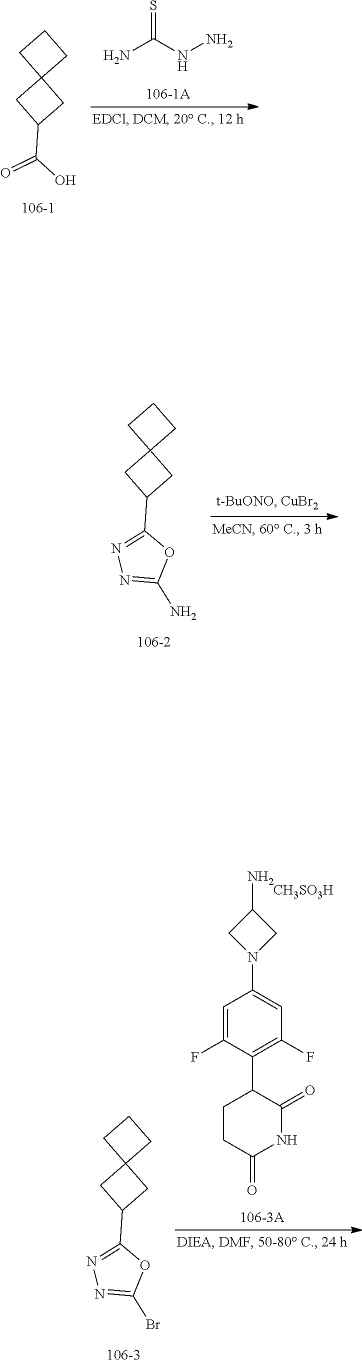

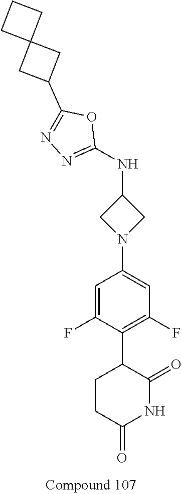 [SMILES: O=C1CCC(c2c(F)cc(N3CC(Nc4nnc(C5CC6(CCC6)C5)o4)C3)cc2F)C(=O)N1]

### Step 1. Procedure for Preparation of Compound 2—5-(spiro[3.3]heptan-2-yl)-1,3,4-oxadiazol-2-amine

To a solution of spiro[3.3]heptane-2-carboxylic acid (2.00 g, 14.3 mmol, 1.00 eq) in dichloromethane (40.0 mL) were added hydrazinecarbothioamide (2.08 g, 22.8 mmol, 1.60 eq) and 1-(3-dimethylaminopropyl)-3-ethylcarbodiimide hydrochloride (8.21 g, 42.8 mmol, 3.00 eq). The reaction mixture was stirred at 20° C. for 12 h under nitrogen atmosphere. The reaction mixture was concentrated under reduced pressure to give a residue. The residue was diluted with dimethylformamide (30 mL) and then filtered. The filtrate was purified by reverse phase chromatography (C18, 330 g; condition: water/acetonitrile=100:0 to 90:10, 0.1% ammonium hydroxide) and concentrated under reduced pressure to afford 5-(spiro[3.3]heptan-2-yl)-1,3,4-oxadiazol-2-amine (2.70 g, crude) as black oil.

¹H NMR (400 MHz, DMSO-d₆) δ=3.34 (br d, J=8.5 Hz, 1H), 2.37-2.30 (m, 2H), 2.23-2.14 (m, 2H), 2.08-2.02 (m, 2H), 1.94-1.84 (m, 2H), 1.80-1.71 (m, 2H).

### Step 2. Procedure for Preparation of Compound 3—2-bromo-5-(spiro[3.3]heptan-2-yl)-1,3,4-oxadiazole

To a solution of 5-(spiro[3.3]heptan-2-yl)-1,3,4-oxadiazol-2-amine (2.40 g, 13.4 mmol, 1.00 eq) in acetonitrile (30.0 mL) were added copper bromide (4.49 g, 20.1 mmol, 941 μL, 1.50 eq) and tert-butyl nitrite (2.07 g, 20.1 mmol, 2.39 mL, 1.50 eq). The reaction mixture was stirred at 60° C. for 3 h under nitrogen atmosphere. The reaction mixture was filtered and the filtrate was concentrated under reduced pressure to give a residue. The residue was purified by column chromatography (SiO₂, petroleum ether/ethyl acetate=1/0 to 20/1) to afford 2-bromo-5-(spiro[3.3]heptan-2-yl)-1,3,4-oxadiazole (800 mg, 3.29 mmol, 25% yield) as pink oil.

¹H NMR (400 MHz, CDCl₃-d) δ=3.64-3.52 (m, 1H), 2.48-2.37 (m, 4H), 2.12 (t, J=7.3 Hz, 2H), 2.02-1.96 (m, 2H), 1.89-1.82 (m, 2H).

### Step 3. Procedure for Preparation of 3-(2,6-difluoro-4-(3-((5-(spiro[3.3]heptan-2-yl)-1,3,4-oxadiazol-2-yl)amino)azetidin-1-yl)phenyl)piperidine-2,6-dione

To a solution of 3-(4-(3-aminoazetidin-1-yl)-2,6-difluorophenyl)piperidine-2,6-dione (100 mg, 256 umol, 1.00 eq, mesylate) in dimethylformamide (1.00 mL) were added N,N-diisopropylethylamine (66.0 mg, 511 umol, 89.0 uL, 2.00 eq) and 2-bromo-5-(spiro[3.3]heptan-2-yl)-1,3,4-oxadiazole (74.5 mg, 307 umol, 1.20 eq). The mixture was stirred at 50° C. for 12 h. The reaction mixture was stirred at 80° C. for 1 h. 3-(4-(3-aminoazetidin-1-yl)-2,6-difluorophenyl)piperidine-2,6-dione (30.0 mg, 76.7 umol, 0.300 eq, mesylate) and N,N-diisopropylethylamine (19.8 mg, 153 umol, 26.7 uL, 0.600 eq) were added to the mixture and the mixture was stirred at 80° C. for 11 h. The reaction mixture was filtered. The filtrate was purified by Prep-HPLC (column: Phenomenex luna C18 150*25 mm*10 um; mobile phase: [water (formic acid)-acetonitrile]; B %: 40%-70%, 9 min) and lyophilized to afford 3-(2,6-difluoro-4-(3-((5-(spiro[3.3]heptan-2-yl)-1,3,4-oxadiazol-2-yl)amino)azetidin-1-yl)phenyl)piperidine-2,6-dione (23.77 mg, 51.44 umol, 20% yield, 99% purity) as an off-white solid.

¹H NMR (400 MHz, DMSO-d₆) δ=10.86 (s, 1H), 8.12 (d, J=7.0 Hz, 1H), 6.20 (s, 1H), 6.17 (s, 1H), 4.48-4.38 (m, 1H), 4.16 (t, J=7.6 Hz, 2H), 4.04 (dd, J=5.1, 12.6 Hz, 1H), 3.76-3.68 (m, 2H), 3.40 (quin, J=8.4 Hz, 1H), 2.84-2.72 (m, 1H), 2.53-2.52 (m, 1H), 2.38-2.32 (m, 2H), 2.24-2.16 (m, 2H), 2.11-2.02 (m, 3H), 1.95 (td, J=2.6, 5.3 Hz, 1H), 1.93-1.88 (m, 2H), 1.82-1.72 (m, 2H). MS (ESI) m/z 458.3 [M+H]⁺

### Example 107. Synthesis of Compound 108

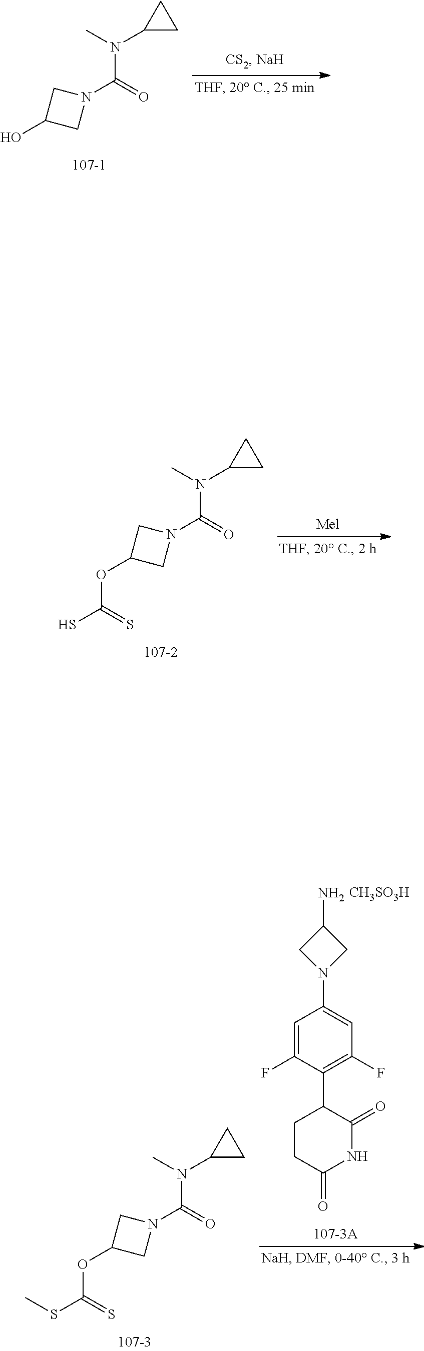

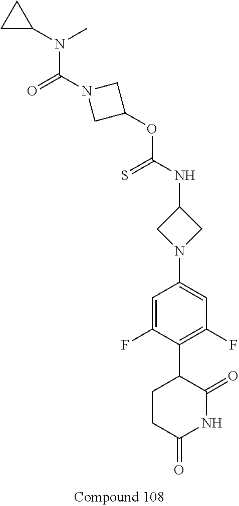 [SMILES: CN(C(=O)N1CC(OC(=S)NC2CN(c3cc(F)c(C4CCC(=O)NC4=O)c(F)c3)C2)C1)C1CC1]

### Step 1. Procedure for Preparation of Compound 2—O-(1-(cyclopropyl(methyl)carbamoyl)azetidin-3-yl) S-hydrogen carbonodithioate

To a solution of N-cyclopropyl-3-hydroxy-N-methylazetidine-1-carboxamide (90.0 mg, 529 umol, 1.00 eq) in tetrahydrofuran (1.00 mL) was added sodium hydride (25.4 mg, 635 umol, 60% purity, 1.20 eq). The reaction mixture was stirred at 20° C. for 20 min. Then, carbon disulfide (60.4 mg, 793 umol, 47.9 uL, 1.50 eq) was added into the mixture. The reaction mixture was stirred at 20° C. for 5 min to afford O-(1-(cyclopropyl(methyl)carbamoyl)azetidin-3-yl) S-hydrogen carbonodithioate (131 mg, crude) in tetrahydrofuran (1.00 mL).

### Step 2. Procedure for Preparation of Compound 3—O-(1-(cyclopropyl(methyl)carbamoyl)azetidin-3-yl) S-methyl carbonodithioate

To a solution of O-(1-(cyclopropyl(methyl)carbamoyl)azetidin-3-yl) S-hydrogen carbonodithioate (130 mg, 529 umol, 1.00 eq) in tetrahydrofuran (1.00 mL) was added iodomethane (90.1 mg, 634 umol, 39.5 uL, 1.20 eq). The reaction mixture was stirred at 20° C. for 2 h. The reaction mixture was quenched with saturated ammonium chloride (1 mL), filtered and concentrated under reduced pressure to give a residue. The residue was purified by column chromatography (SiO₂, petroleum ether/ethyl acetate=1/0 to 0/1) and concentrated under reduced pressure to afford O-(1-(cyclopropyl(methyl)carbamoyl)azetidin-3-yl) S-methyl carbonodithioate (70.0 mg, 269 umol, 51% yield) as colorless oil.

¹H NMR (400 MHz, DMSO-d₆) δ=5.61 (tt, J=4.0, 6.6 Hz, 1H), 4.33 (ddd, J=0.9, 6.7, 10.1 Hz, 2H), 4.01-3.97 (m, 2H), 2.73 (s, 3H), 2.59 (s, 3H), 2.58-2.55 (m, 1H), 0.75-0.69 (m, 2H), 0.64-0.59 (m, 2H).

### Step 3. Procedure for Preparation of O-(1-(cyclopropyl(methyl)carbamoyl)azetidin-3-yl) (1-(4-(2,6-dioxopiperidin-3-yl)-3,5-difluorophenyl)azetidin-3-yl)carbamothioate

To a solution of 3-(4-(3-aminoazetidin-1-yl)-2,6-difluorophenyl)piperidine-2,6-dione (75.2 mg, 192 umol, 1.00 eq, mesylate) in dimethylformamide (2.00 mL) was added O-(1-(cyclopropyl(methyl)carbamoyl)azetidin-3-yl) S-methyl carbonodithioate (50.0 mg, 192 umol, 1.00 eq) and sodium hydride (15.4 mg, 384 umol, 60% purity, 2.00 eq) at 0° C. The reaction mixture was stirred at 40° C. for 3 h. The reaction mixture was quenched by ice cold water (5 mL), extracted with ethyl acetate (2×5 mL). The combined organic layers were washed with brine (10 mL), dried over anhydrous sodium sulfate and concentrated under reduce pressure to give a residue. The residue was purified by Prep-HPLC (column: Waters xbridge 150*25 mm 10 um; mobile phase: [water (ammonium bicarbonate)-acetonitrile]; B %: 26%-56%, 8 min) and lyophilized to afford O-(1-(cyclopropyl(methyl)carbamoyl)azetidin-3-yl) (1-(4-(2,6-dioxopiperidin-3-yl)-3,5-difluorophenyl)azetidin-3-yl)carbamothioate (12.74 mg, 24.85 umol, 13% yield, 99% purity) as a white solid.

¹H NMR (400 MHz, DMSO-d₆) δ=10.86 (s, 1H), 10.08 (br d, J=4.3 Hz, 1H), 6.19 (s, 1H), 6.16 (s, 1H), 5.43-5.36 (m, 1H), 4.74-4.66 (m, 1H), 4.28 (dd, J=6.7, 9.8 Hz, 2H), 4.15 (br t, J=7.6 Hz, 2H), 4.03 (br dd, J=4.9, 12.4 Hz, 1H), 3.95-3.86 (m, 2H), 3.77-3.69 (m, 2H), 2.82-2.76 (m, 1H), 2.74-2.71 (m, 3H), 2.62-2.54 (m, 2H), 2.13-2.03 (m, 1H), 1.98-1.91 (m, 1H), 0.75-0.69 (m, 2H), 0.65-0.59 (m, 2H). MS (ESI) m/z. 508.2 [M+H]⁺.

### Example 108. Synthesis of Compound 109

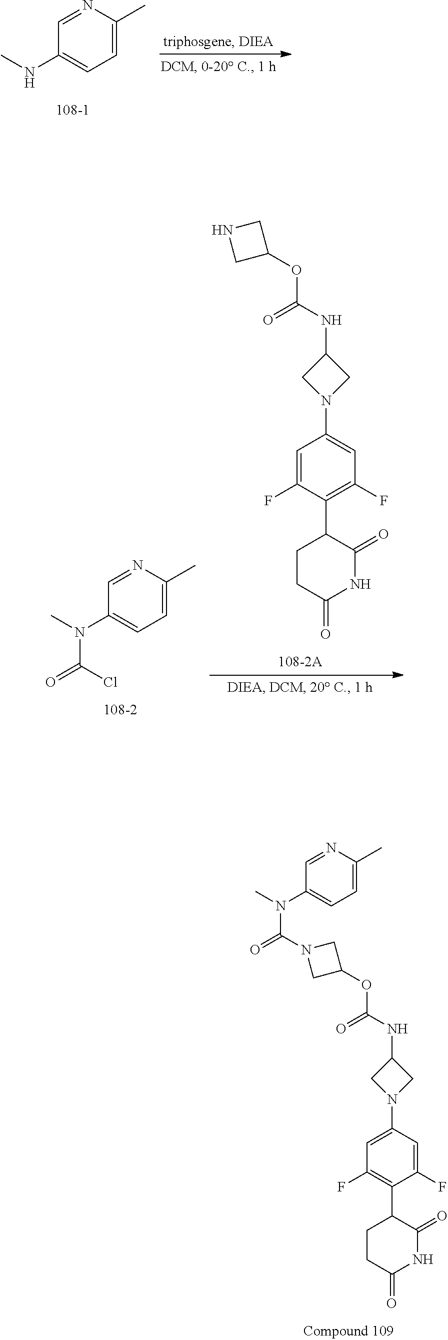

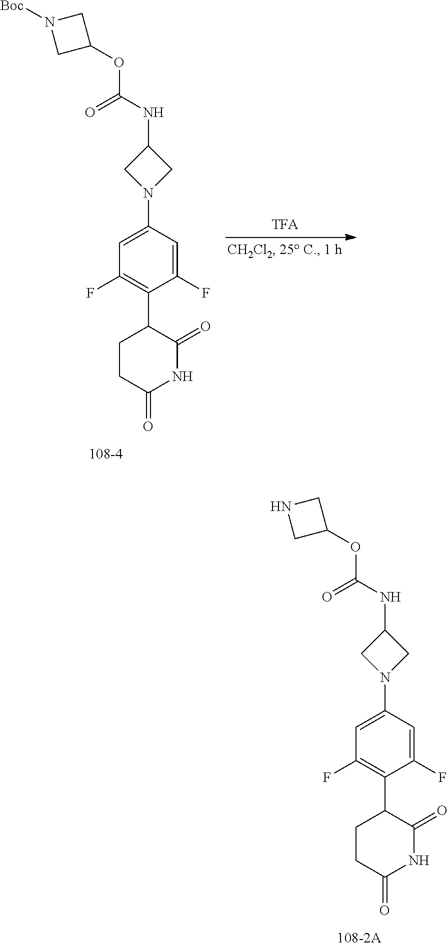 [SMILES: CC(C)(C)OC(=O)N1CC(OC(=O)NC2CN(c3cc(F)c(C4CCC(=O)NC4=O)c(F)c3)C2)C1 ; O=C1CCC(c2c(F)cc(N3CC(NC(=O)OC4CNC4)C3)cc2F)C(=O)N1]

### Step 1. Procedure for Preparation of 2A—azetidin-3-yl (1-(4-(2,6-dioxopiperidin-3-yl)-3,5-difluorophenyl) azetidin-3-yl) carbamate

To a solution of tert-butyl 3-(((1-(4-(2,6-dioxopiperidin-3-yl)-3,5-difluorophenyl) azetidin-3-yl) carbamoyl) oxy) azetidine-1-carboxylate (100 mg, 202 umol, 1.00 eq) in dichloromethane (1.50 mL) was added trifluoroacetic acid (46.1 mg, 404 umol, 29.9 uL, 2.00 eq). The mixture was stirred at 25° C. for 1 hr. The reaction mixture was concentrated under reduced pressure to afford compound azetidin-3-yl (1-(4-(2,6-dioxopiperidin-3-yl)-3,5-difluorophenyl) azetidin-3-yl) carbamate (300 mg, crude) was obtained as a white solid.

### Step 2. Procedure for Preparation of Compound 2—methyl (6-methylpyridin-3-yl) carbamic chloride

To a solution of N, 6-dimethylpyridin-3-amine (50.0 mg, 409 umol, 1.00 eq) in dichloromethane (5.00 mL) were added N, N-diisopropylethylamine (105 mg, 818 umol, 142 uL, 2.00 eq) and triphosgene (121 mg, 409 umol, 1.00 eq) at 0° C. Then the mixture was stirred at 20° C. for 1 h. The reaction mixture was filtered and concentrated under reduced pressure to afford compound methyl(6-methylpyridin-3-yl)carbamic chloride (75.0 mg, crude) was obtained as a white solid.

### Step 3. Procedure for Preparation of 1-(methyl (6-methylpyridin-3-yl) carbamoyl) azetidin-3-yl (1-(4-(2,6-dioxopiperidin-3-yl)-3,5-difluorophenyl) azetidin-3-yl) carbamate

To a solution of N-methyl-N-(6-methyl-3-pyridyl)carbamoyl chloride (23.4 mg, 126 umol, 1.00 eq) and azetidin-3-yl (1-(4-(2,6-dioxopiperidin-3-yl)-3,5-difluorophenyl)azetidin-3-yl)carbamate (50.0 mg, 126 umol, 1.00 eq) in dichloromethane (5.00 mL) was added N,N-diisopropylethylamine (18.0 mg, 139 umol, 24.2 uL, 1.10 eq). The mixture was stirred at 20° C. for 1 hr. The reaction mixture was filtered and concentrated under reduced pressure to give a residue. The residue was purified by Prep-HPLC (column: Phenomenex Luna C18 150*25 mm*10 um; mobile phase: [water(formic acid)-acetonitrile]; B %: 10%-40%, 10 min) to afford 1-(methyl(6-methylpyridin-3-yl)carbamoyl)azetidin-3-yl-(1-(4-(2,6-dioxopiperidin-3-yl)-3,5-difluorophenyl)azetidin-3-yl)carbamate (40.75 mg, 72.86 umol, 57% yield, 97% purity) was obtained as a yellow solid.

1H NMR (400 MHz, DMSO-d6) δ=10.86 (s, 1H), 8.36 (d, J=2.4 Hz, 1H), 8.07 (br d, J=7.6 Hz, 1H), 7.60 (dd, J=2.4, 8.3 Hz, 1H), 7.29 (d, J=8.4 Hz, 1H), 6.14 (br d, J=12.0 Hz, 2H), 4.88-4.79 (m, 1H), 4.42-4.32 (m, 1H), 4.10-4.00 (m, 3H), 3.79 (br dd, J=6.8, 9.4 Hz, 2H), 3.61 (br t, J=6.8 Hz, 2H), 3.40 (br dd, J=3.6, 9.8 Hz, 2H), 3.13 (s, 3H), 2.83-2.74 (m, 1H), 2.60-2.57 (m, 1H), 2.47 (s, 3H), 2.07 (dq, J=3.2, 13.0 Hz, 1H), 1.99-1.89 (m, 1H). MS (ESI) m/z 543.2 [M+H]+.

### Example 109. Synthesis of Compound 110

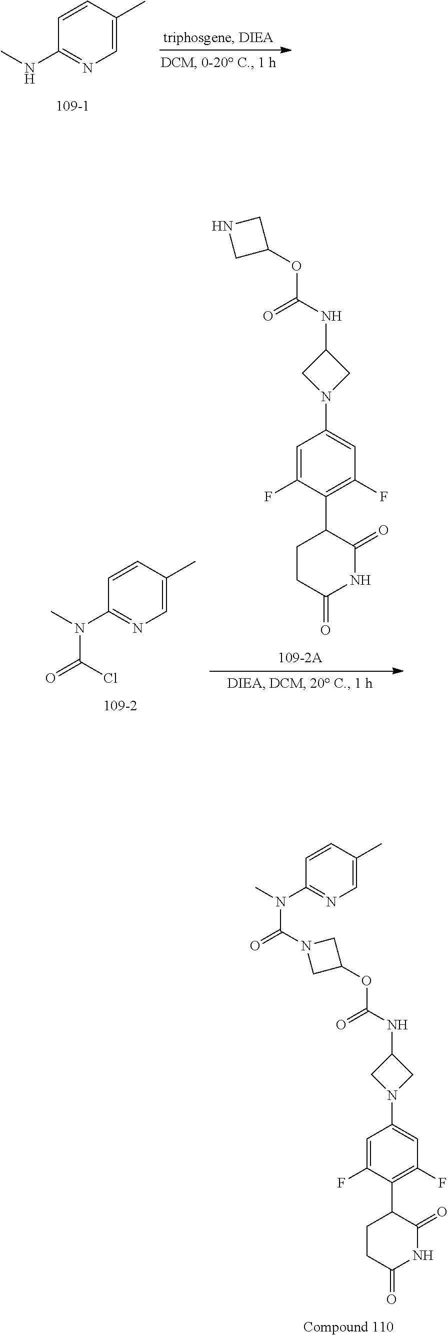

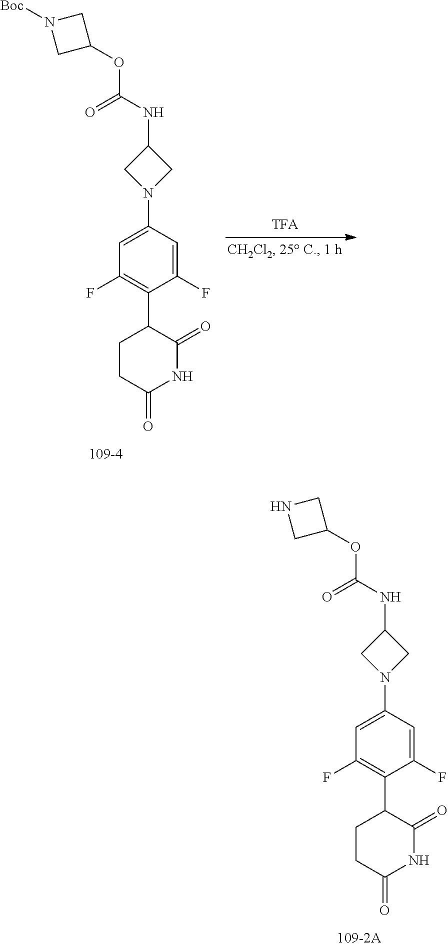 [SMILES: CC(C)(C)OC(=O)N1CC(OC(=O)NC2CN(c3cc(F)c(C4CCC(=O)NC4=O)c(F)c3)C2)C1 ; O=C1CCC(c2c(F)cc(N3CC(NC(=O)OC4CNC4)C3)cc2F)C(=O)N1]

### Step 1. Procedure for Preparation of 2A—azetidin-3-yl (1-(4-(2,6-dioxopiperidin-3-yl)-3,5-difluorophenyl) azetidin-3-yl) carbamate

To a solution of tert-butyl 3-(((1-(4-(2,6-dioxopiperidin-3-yl)-3,5-difluorophenyl) azetidin-3-yl) carbamoyl) oxy) azetidine-1-carboxylate (100 mg, 202 umol, 1.00 eq) in dichloromethane (1.50 mL) was added trifluoroacetic acid (46.1 mg, 404 umol, 29.9 uL, 2.00 eq). The mixture was stirred at 25° C. for 1 hr. The reaction mixture was concentrated under reduced pressure to afford azetidin-3-yl (1-(4-(2,6-dioxopiperidin-3-yl)-3,5-difluorophenyl) azetidin-3-yl) carbamate (300 mg, crude) was obtained as a white solid.

### Step 2. Procedure for Preparation of Compound 2 methyl (5-methylpyridin-2-yl) carbamic chloride

To a solution of N, 5-dimethylpyridin-2-amine (50.0 mg, 409 umol, 1.00 eq) in dichloromethane (1.00 mL) were added N, N-diisopropylethylamine (79.3 mg, 613 umol, 106 uL, 1.50 eq) and triphosgene (121 mg, 409 umol, 1.00 eq) at 0° C. Then the mixture was stirred at 20° C. for 1 h. The reaction mixture was filtered and concentrated under reduced pressure to afford methyl(5-methylpyridin-2-yl)carbamic chloride (50.0 mg, crude) was obtained as a yellow solid.

### Step 3. Procedure for Preparation of 1-(methyl (5-methylpyridin-2-yl) carbamoyl) azetidin-3-yl (1-(4-(2,6-dioxopiperidin-3-yl)-3,5-difluorophenyl) azetidin-3-yl) carbamate

To a solution of methyl(5-methylpyridin-2-yl)carbamic chloride (46.8 mg, 253 umol, 1.00 eq) and azetidin-3-yl (1-(4-(2,6-dioxopiperidin-3-yl)-3,5-difluorophenyl)azetidin-3-yl)carbamate (100 mg, 253 umol, 1.00 eq) in dichloromethane (2.00 mL) was added N,N-diisopropylethylamine (36.0 mg, 278 umol, 48.5 uL, 1.10 eq). The mixture was stirred at 20° C. for 1 hr. The reaction mixture was filtered and concentrated under reduced pressure to give a residue. The residue was purified by Prep-HPLC (column: Phenomenex Luna C18 150*25 mm*10 um; mobile phase: [water (formic acid)-acetonitrile]; B %: 26%-56%, 10 min) to afford 1-(methyl(5-methylpyridin-2-yl)carbamoyl)azetidin-3-yl-(1-(4-(2,6-dioxopiperidin-3-yl)-3,5-difluorophenyl)azetidin-3-yl)carbamate (54.56 mg, 99.56 umol, 39% yield, 99% purity) was obtained as a off-white solid

1H NMR (400 MHz, DMSO-d6) δ=10.87 (s, 1H), 8.23 (d, J=2.4 Hz, 1H), 8.09 (d, J=7.2 Hz, 1H), 7.62 (dd, J=2.4, 8.4 Hz, 1H), 7.26 (d, J=8.0 Hz, 1H), 6.15 (d, J=12.0 Hz, 2H), 4.96-4.89 (m, 1H), 4.43-4.33 (m, 1H), 4.11-4.00 (m, 3H), 3.94 (dd, J=7.2, 9.5 Hz, 2H), 3.62 (br t, J=6.8 Hz, 2H), 3.53 (dd, J=4.0, 10.0 Hz, 2H), 3.20 (s, 3H), 2.84-2.73 (m, 1H), 2.60-2.57 (m, 1H), 2.27 (s, 3H), 2.11-2.04 (m, 1H), 1.97-1.92 (m, 1H). MS (ESI) m/z 543.4 [M+H]+.

### Example 110. Synthesis of Compound 111

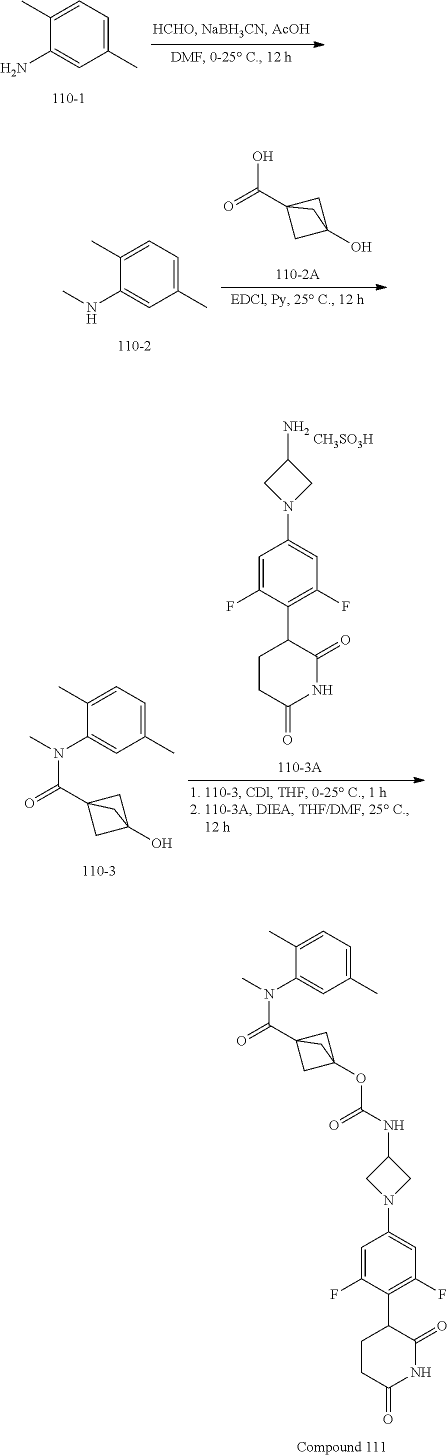

### Step 1. Procedure for Preparation of Compound 2—N, 2,5-trimethylaniline

To a solution of 2,5-dimethylaniline (2.00 g, 16.50 mmol, 1 eq) in dimethylformamide (20.0 mL) was added formaldehyde (1.07 g, 13.2 mmol, 983 uL, 37% purity, 0.800 eq), sodium cyanoborohydride (3.11 g, 49.5 mmol, 3.00 eq) and acetic acid (99.1 mg, 1.65 mmol, 94.4 uL, 0.100 eq) at 0° C. The reaction mixture was stirred at 25° C. for 12 h. The mixture was diluted with water (20.0 mL), extracted with ethyl acetate (3×20.0 mL), washed with brine (3×20.0 mL), and dried over anhydrous sodium sulfate, filtered and concentrated under reduced pressure to give a residue. The residue was purified by column chromatography (SiO2, petroleum ether/ethyl acetate=10/1) to afford N, 2,5-trimethylaniline (1.08 g, 7.99 mmol, 48% yield) as a colorless solid.

1H NMR (400 MHz, CHCl3-d) δ=6.95 (d, J=7.4 Hz, 1H), 6.50 (d, J=7.3 Hz, 1H), 6.46 (s, 1H), 2.90 (s, 3H), 2.32 (s, 3H), 2.11 (s, 3H).

### Step 2. Procedure for Preparation of Compound 3—N-(2,5-dimethylphenyl)-3-hydroxy-N-methylbicyclo[1.1.1]pentane-1-carboxamide

To a solution of 3-hydroxybicyclo[1.1.1]pentane-1-carboxylic acid (100 mg, 780 umol, 1.00 eq) in pyridine (1.00 mL) was added 1-(3-dimethylaminopropyl)-3-ethylcarbodiimide hydrochloride (224 mg, 1.17 mmol, 1.50 eq) and N, 2,5-trimethylaniline (137 mg, 1.01 mmol, 1.30 eq). The mixture was stirred at 25° C. for 12 h. The mixture was concentrated under reduced pressure to give a residue. The residue diluted with water (20.0 mL), extracted with ethyl acetate (3×20.0 mL), washed with brine (3×20.0 mL), and dried over anhydrous sodium sulfate, filtered and concentrated under reduced pressure to give a residue. The residue was purified prep-TLC (SiO2, petroleum ether/ethyl acetate=0/1) to afford N-(2,5-dimethylphenyl)-3-hydroxy-N-methylbicyclo[1.1.1]pentane-1-carboxamide (100 mg, 408 umol, 52% yield) as a yellow solid.

1H NMR (400 MHz, CHCl3-d) δ=7.17-7.12 (m, 1H), 7.11-7.07 (m, 1H), 6.91 (s, 1H), 3.16 (s, 3H), 2.33 (s, 3H), 2.17 (s, 3H), 1.85-1.79 (m, 3H), 1.76-1.72 (m, 3H).

### Step 3. Procedure for Preparation of 3-(4-(3-((5-(2,6-dimethyl-4-(trifluoromethoxy)phenyl)-1,3,4-oxadiazol-2-yl)amino)azetidin-1-yl)-2,6-difluorophenyl) piperidine-2,6-dione

To a solution of N-(2,5-dimethylphenyl)-3-hydroxy-N-methylbicyclo[1.1.1]pentane-1-carboxamide (70.0 mg, 285 umol, 1.00 eq) in tetrahydrofuran (1.00 mL) was added di(1H-imidazol-1-yl)methanone (55.5 mg, 342 umol, 1.20 eq) at 0° C. The mixture was stirred at 25° C. for 1 h. The resulting solution was added into a mixture of 3-(4-(3-aminoazetidin-1-yl)-2,6-difluorophenyl)piperidine-2,6-dione (105 mg, 268 umol, 1.00 eq, mesylate), 2,3,4,6,7,8,9,10-octahydropyrimido[1,2-a]azepine (40.8 mg, 268 umol, 40.4 uL, 1.00 eq) and N,N-diisopropylethylamine (69.4 mg, 537 umol, 93.5 uL, 2.00 eq) in tetrahydrofuran (1.00 mL) and dimethylformamide (1.00 mL). The reaction mixture was stirred at 25° C. for 12 h. The reaction mixture was concentrated under reduced pressure to give a residue. The residue was diluted with dimethylformamide (1.00 mL) and then filtered. The filtrate was purified by Prep-HPLC (column: Phenomenex luna C18 150*25 mm*10 um; mobile phase: [water(formic acid)-acetonitrile]; B %: 43%-73%, 10 min) and lyophilized to afford 3-((2,5-dimethylphenyl)(methyl)carbamoyl)bicyclo[1.1.1]pentan-1-yl (1-(4-(2,6-dioxopiperidin-3-yl)-3,5-difluorophenyl)azetidin-3-yl)carbamate (22.91 mg, 40.03 umol, 14.92% yield, 99% purity) as a white solid.

1H NMR (400 MHz, DMSO-d6) δ=10.84 (s, 1H), 7.82 (d, J=7.5 Hz, 1H), 7.24-7.19 (m, 1H), 7.18-7.12 (m, 1H), 7.02 (s, 1H), 6.12 (br d, J=11.0 Hz, 2H), 4.36-4.25 (m, 1H), 4.06-4.02 (m, 2H), 4.01 (br s, 1H), 3.55 (br t, J=6.8 Hz, 2H), 3.04 (s, 3H), 2.80-2.72 (m, 1H), 2.47 (br d, J=2.8 Hz, 1H), 2.29 (s, 3H), 2.11 (s, 3H), 2.08-2.00 (m, 1H), 1.94-1.89 (m, 1H), 1.88-1.83 (m, 3H), 1.82-1.75 (m, 3H). MS (ESI) m/z 567.2 [M+H]+.

### Example 111. Synthesis of Compound 112

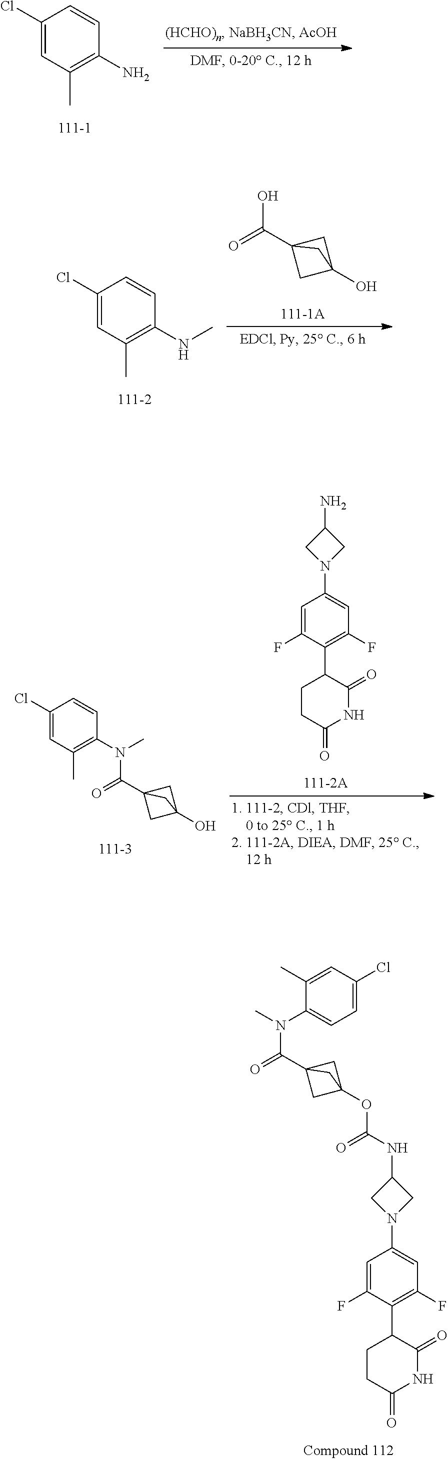

### Step 1. Procedure for Preparation of Compound 2—4-chloro-N, 2-dimethylaniline

To a solution of 4-chloro-2-methylaniline (2.00 g, 14.1 mmol, 1.00 eq) in dimethylformamide (10.0 mL) was added formaldehyde (917 mg, 11.3 mmol, 841 uL, 37% purity, 0.8 eq), sodium triacetoxyhydroborate (8.98 g, 42.4 mmol, 3.00 eq) and acetic acid (84.8 mg, 1.41 mmol, 80.8 uL, 0.100 eq). The mixture was stirred at 20° C. for 12 h. The reaction mixture was quenched by addition water 50 mL at 20° C., and then extracted with ethyl acetate 120 mL (40 mL*3). The combined organic layers were washed with brine 50 mL, dried over sodium sulfate, filtered and concentrated under reduced pressure to give a residue. The residue was purified by column chromatography (SiO2, Petroleum ether/Ethyl acetate=40/1) to afford 4-chloro-N, 2-dimethylaniline (500 mg, 3.21 mmol, 23% yield) as yellow oil.

1H NMR (400 MHz, DMSO-d6) δ=7.04 (dd, J=2.4, 8.6 Hz, 1H), 6.99 (d, J=2.1 Hz, 1H), 6.43 (d, J=8.5 Hz, 1H), 5.15 (br d, J=4.4 Hz, 1H), 2.70 (d, J=4.9 Hz, 3H), 2.05 (s, 3H)

### Step 2. Procedure for Preparation of Compound 3—N-(4-chloro-2-methylphenyl)-3-hydroxy-N-methylbicyclo[1.1.1]pentane-1-carboxamide

To a solution of 3-hydroxybicyclo[1.1.1]pentane-1-carboxylic acid (100 mg, 781 umol, 1.00 eq) in pyridine (1.00 mL) was added 1-(3-dimethylaminopropyl)-3-ethylcarbodiimide hydrochloride (224 mg, 1.17 mmol, 1.50 eq) and 4-chloro-N, 2-dimethylaniline (146 mg, 937 umol, 1.20 eq). The mixture was stirred at 25° C. for 6 h. The reaction mixture was concentrated under reduced pressure to give a residue. The residue was purified by column chromatography (SiO2, Petroleum ether/Ethyl acetate=1:1) to afford N-(4-chloro-2-methylphenyl)-3-hydroxy-N-methylbicyclo[1.1.1]pentane-1-carboxamide (50 mg, 188.16 umol, 24% yield) as yellow oil. MS (ESI) m/z 266.0 [M+H]+

### Step 3. Procedure for Preparation of 3-((4-chloro-2-methylphenyl)(methyl)carbamoyl)bicyclo[1.1.1]pentan-1-yl (1-(4-(2,6-dioxopiperidin-3-yl)-3,5-difluorophenyl)azetidin-3-yl)carbamate

To a solution of N-(4-chloro-2-methyl-phenyl)-3-hydroxy-N-methyl-bicyclo[1.1.1]pentane-1-carboxamide (54.0 mg, 203 umol, 1.00 eq) in tetrahydrofuran (1.00 mL) was added di(1H-imidazol-1-yl)methanone (36.3 mg, 224 umol, 1.10 eq) at 25° C. The mixture was stirred at 25 C for 1 h to give a residue. To a solution of 3-(4-(3-aminoazetidin-1-yl)-2,6-difluorophenyl)piperidine-2,6-dione (65.9 mg, 223 umol, 1.10 eq) in dimethyl formamide (2.00 mL) was added N,N-diisopropylethylamine (78.7 mg, 609 umol, 106 uL, 3.00 eq) and the above-mentioned residue. The mixture was stirred at 25° C. for 12 h. The reaction mixture was concentrated under reduced pressure to give a residue. The residue was purified by prep-HPLC (column: Phenomenex luna C18 150*25 mm*10 um; mobile phase: [water(FA)-ACN]; B %: 44%-74%, 10 min). and lyophilized to afford 3-((4-chloro-2-methylphenyl)(methyl)carbamoyl)bicyclo[1.1.1]pentan-1-yl (1-(4-(2,6-dioxopiperidin-3-yl)-3,5-difluorophenyl)azetidin-3-yl)carbamate (20.8 mg, 34.6 umol, 17.07% yield, 98% purity) as a white solid.

1H NMR (400 MHz, DMSO-d6) δ=10.85 (br s, 1H), 7.86 (d, J=7.5 Hz, 1H), 7.48 (d, J=2.0 Hz, 1H), 7.38-7.33 (m, 1H), 7.31-7.27 (m, 1H), 6.13 (d, J=11.1 Hz, 2H), 4.37-4.26 (m, 1H), 4.08-4.00 (m, 3H), 3.56 (br t, J=6.7 Hz, 2H), 3.04 (s, 3H), 2.85-2.74 (m, 1H), 2.48 (br s, 1H), 2.17 (s, 3H), 2.10-2.01 (m, 1H), 1.98-1.93 (m, 1H), 1.90 (d, J=9.1 Hz, 3H), 1.85-1.78 (m, 3H). MS (ESI) m/z 587.3 [M+H]+.

### Example 112. Synthesis of Compound 113

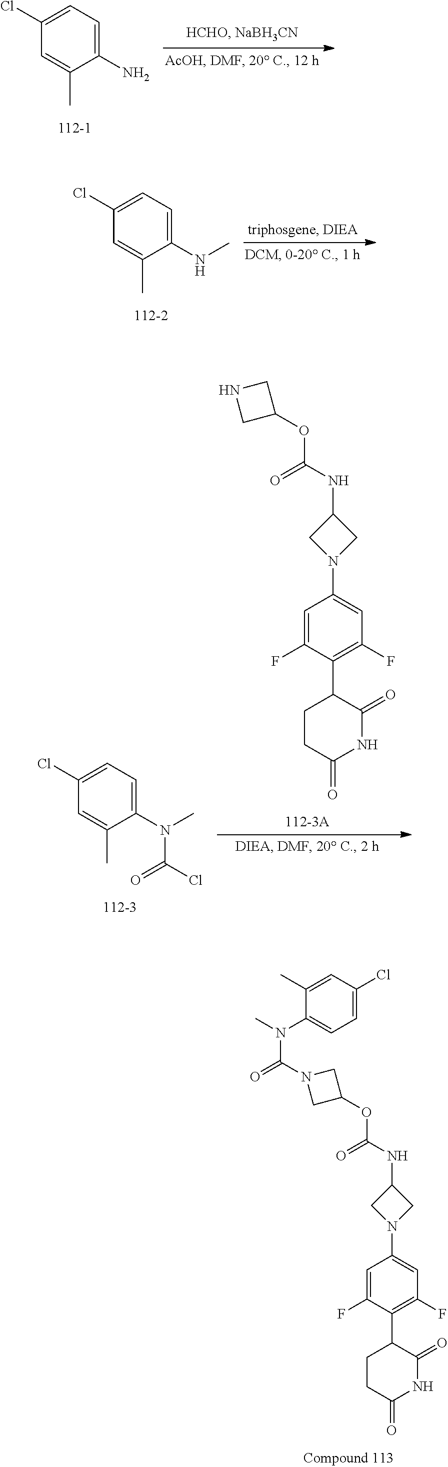

### Step 1. Procedure for Compound 2—4-chloro-N, 2-dimethylaniline

To a solution of 4-chloro-2-methylaniline (3.00 g, 21.2 mmol, 1.00 eq) in N,N-dimethyl formamide (30.0 mL) was added formaldehyde (1.38 g, 17.0 mmol, 1.26 mL, 37% purity, 0.800 eq), sodium cyanoborohydride (2.66 g, 42.4 mmol, 2.00 eq) and acetic acid (127 mg, 2.12 mmol, 121 uL, 0.100 eq). The mixture was stirred at 20° C. for 12 h. The reaction mixture was quenched with water (50 mL) and extracted with ethyl acetate (3×50 mL). The combined organic layer was washed with water (2×50 mL), dried over anhydrous sodium sulfate, filtered and concentrated under reduced pressure. The residue was purified by chromatography on silica gel (petroleum ether:ethyl acetate=1/0 to 10/1) to afford 4-chloro-N, 2-dimethyl-aniline (0.569 g, 3.66 mmol, 17% yield) as yellow oil.

1H NMR (400 MHz, CDCl3-d) δ=7.00 (dd, J=2.4, 8.5 Hz, 1H), 6.92 (d, J=2.1 Hz, 1H), 6.40 (d, J=8.5 Hz, 1H), 2.76 (s, 3H), 1.99 (s, 3H).

### Step 2. Procedure for Compound 3—(4-chloro-2-methylphenyl)(methyl)carbamic chloride

To a solution of 4-chloro-N, 2-dimethylaniline (35.0 mg, 225 umol, 1.00 eq) in dichloromethane (1.00 mL) was added N,N-diisopropylethylamine (58.1 mg, 450 umol, 78.4 uL, 2.00 eq) and triphosgene (66.7 mg, 225 umol, 1.00 eq) at 0° C. The reaction mixture was stirred at 20° C. for 1 h. The reaction mixture was concentrated under reduced pressure to afford (4-chloro-2-methylphenyl)(methyl)carbamic chloride (49.0 mg, 225 umol, 99% yield) as yellow oil.

### Step 3. Procedure for 1-((4-chloro-2-methylphenyl)(methyl)carbamoyl)azetidin-3-yl (1-(4-(2,6-dioxopiperidin-3-yl)-3,5-difluorophenyl)azetidin-3-yl)carbamate

To a solution of azetidin-3-yl (1-(4-(2,6-dioxopiperidin-3-yl)-3,5-difluorophenyl)azetidin-3-yl)carbamate (57.5 mg, 146 umol, 1.00 eq) in N,N-dimethyl formamide (1.00 mL) was added N,N-diisopropylethylamine (37.7 mg, 292 umol, 50.8 uL, 2.00 eq) and (4-chloro-2-methylphenyl)(methyl)carbamic chloride (35.0 mg, 160 umol, 1.10 eq). The mixture was stirred at 20° C. for 2 h. The mixture was concentrated under reduced pressure. The residue was purified by Prep-HPLC (column: Phenomenex luna C18 150*25 mm*10 um; mobile phase: [water(formic acid)-acetonitrile]; B %: 38%-68%, 10 min) and lyophilized to afford 1-((4-chloro-2-methylphenyl)(methyl)carbamoyl)azetidin-3-yl-(1-(4-(2,6-dioxopiperidin-3-yl)-3,5-difluorophenyl) azetidin-3-yl)carbamate (23.22 mg, 39.51 umol, 27% yield, 98% purity) as a white solid.

¹H NMR (400 MHz, DMSO-d6) δ=10.84 (s, 1H), 8.03 (br d, J=7.5 Hz, 1H), 7.40 (d, J=2.3 Hz, 1H), 7.32-7.27 (m, 1H), 7.26-7.20 (m, 1H), 6.12 (d, J=11.1 Hz, 2H), 4.84-4.74 (m, 1H), 4.42-4.29 (m, 1H), 4.12-3.99 (m, 3H), 3.75-3.62 (m, 2H), 3.58 (br t, J=6.8 Hz, 2H), 3.16 (br d, J=5.3 Hz, 2H), 2.99 (s, 3H), 2.80-2.71 (m, 1H), 2.43-2.38 (m, 1H), 2.18 (s, 3H), 2.11-2.02 (m, 1H), 1.97-1.88 (m, 1H). MS (ESI) m/z 576.2 [M+H]+.

### Example 113. Synthesis of Compound 114

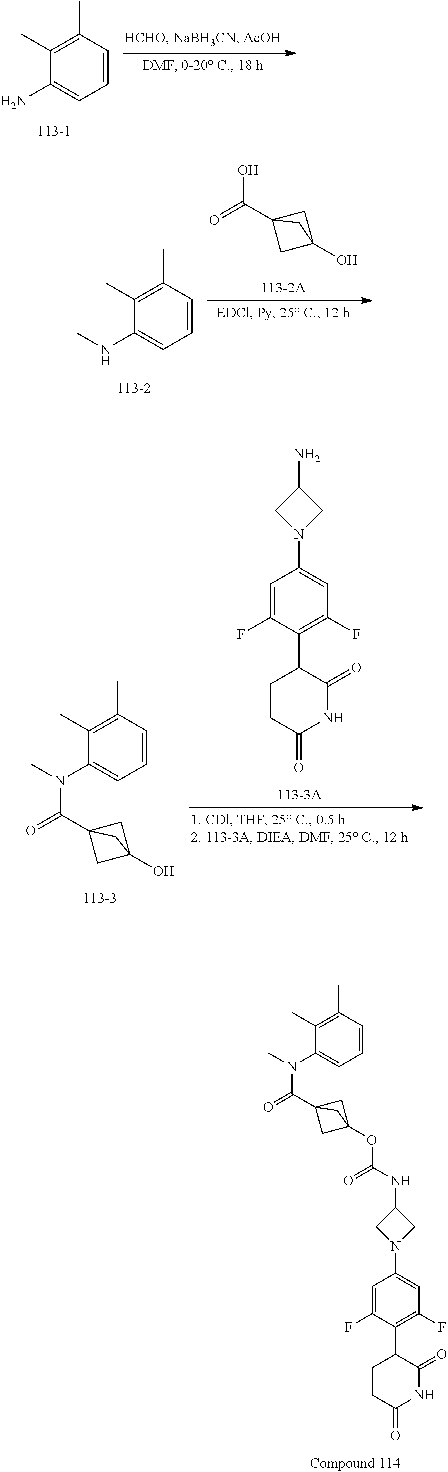

### Step 1. Procedure for Preparation of Compound 2—N, 2,3-trimethylaniline

To a solution of 2,3-dimethylaniline (1.00 g, 8.25 mmol, 1.01 mL, 1.00 eq) in dimethylformamide (10.0 mL) was added sodium cyanoborohydride (1.56 g, 24.8 mmol, 3.00 eq) and acetic acid (49.6 mg, 825 umol, 47.2 uL, 0.100 eq) and formaldehyde (536 mg, 6.60 mmol, 492 uL, 37.0% purity, 0.800 eq) at 0° C. The mixture was stirred at 20° C. for 18 h. The reaction mixture was diluted with water (20.0 mL) and extracted with ethyl acetate (20 mL*2). The combined organic layers were washed with brine, dried over sodium sulfate, filtered and concentrated under reduced pressure to give a residue. The residue was purified by column chromatography (SiO2, petroleum ether/ethyl acetate=1/0 to 10/1) to afford N, 2,3-trimethylaniline (170 mg, 1.09 mmol, 15.5% yield) as yellow oil.

¹H NMR (400 MHz, DMSO-d6) δ=6.89 (t, J=7.8 Hz, 1H), 6.42 (d, J=7.5 Hz, 1H), 6.33 (d, J=8.1 Hz, 1H), 4.90 (br d, J=4.8 Hz, 1H), 2.69 (d, J=5.0 Hz, 3H), 2.17 (s, 3H), 1.95 (s, 3H)

### Step 2. Procedure for Preparation of Compound 3—N-(2,3-dimethylphenyl)-3-hydroxy-N-methylbicyclo[1.1.1]pentane-1-carboxamide

To a solution of 3-hydroxybicyclo[1.1.1]pentane-1-carboxylic acid (80.0 mg, 624 umol, 1.00 eq) in pyridine (1.50 mL) was added 1-(3-dimethylaminopropyl)-3-ethylcarbodiimide hydrochloride (180 mg, 933 umol, 1.50 eq) and N, 2,3-trimethylaniline (101 mg, 749 umol, 1.20 eq). The mixture was stirred at 25° C. for 12 h. The reaction mixture was concentrated under reduced pressure to remove pyridine. The residue was diluted with hydrochloric acid (1 M, 5 mL) and extracted with ethyl acetate (10 mL*2). The combined organic layers were dried over sodium sulfate, filtered and concentrated under reduced pressure to give a residue. The residue was purified by column chromatography (SiO2, petroleum ether/ethyl acetate=1/1) to afford N-(2,3-dimethylphenyl)-3-hydroxy-N-methylbicyclo[1.1.1]pentane-1-carboxamide (70 mg, 285.4 umol, 45.7% yield) as a white solid.

1H NMR (400 MHz, DMSO-d6) δ=7.25-7.19 (m, 1H), 7.18-7.12 (m, 1H), 7.02 (d, J=7.6 Hz, 1H), 6.03 (s, 1H), 3.02 (s, 3H), 2.28 (s, 3H), 2.03 (s, 3H), 1.58-1.52 (m, 3H), 1.51-1.44 (m, 3H)

### Step 3. Procedure for Preparation of 3-((2,3-dimethylphenyl)(methyl)carbamoyl)bicyclo[1.1.1]pentan-1-yl (1-(4-(2,6-dioxopiperidin-3-yl)-3,5-difluorophenyl)azetidin-3-yl)carbamate

To a solution of N-(2,3-dimethylphenyl)-3-hydroxy-N-methylbicyclo[1.1.1]pentane-1-carboxamide (70.0 mg, 285 umol, 1.00 eq) in tetrahydrofuran (1.00 mL) was added di(1H-imidazol-1-yl)methanone (46.3 mg, 285 umol 1.00 eq). The mixture was stirred at 25° C. for 0.5 h. The resulting solution was added into a mixture of 3-(4-(3-aminoazetidin-1-yl)-2,6-difluorophenyl)piperidine-2,6-dione (110.71 mg, 282.86 umol, 1.00 eq, methanesulfonic acid) and N,N-diisopropylethylamine (110 mg, 849 umol, 148 uL, 3.00 eq) in dimethylformamide (1.00 mL). The reaction mixture was stirred at 25° C. for 12 h. The reaction mixture was concentrated under reduced pressure to give a residue. The residue was purified by reverse phase chromatography (C18, 40 g, condition: water/acetonitrile=100:0 to 0:1, 0.1% formic acid) and lyophilized to afford 3-((2,3-dimethylphenyl)(methyl)carbamoyl)bicyclo[1.1.1]pentan-1-yl (1-(4-(2,6-dioxopiperidin-3-yl)-3,5-difluorophenyl)azetidin-3-yl)carbamate (22.99 mg, 40.17 umol, 14.20% yield, 99% purity) as a white solid.

1H NMR (400 MHz, DMSO-d6) δ=10.85 (s, 1H), 7.81 (d, J=7.4 Hz, 1H), 7.27-7.20 (m, 1H), 7.17 (t, J=7.7 Hz, 1H), 7.05 (d, J=7.5 Hz, 1H), 6.12 (d, J=11.0 Hz, 2H), 4.31 (br d, J=6.4 Hz, 1H), 4.03 (br t, J=7.4 Hz, 3H), 3.57-3.52 (m, 2H), 3.04 (s, 3H), 2.80-2.73 (m, 1H), 2.62-2.57 (m, 1H), 2.29 (s, 3H), 2.12-2.06 (m, 1H), 2.05 (s, 3H), 1.98-1.91 (m, 1H), 1.87-1.82 (m, 3H), 1.80-1.73 (m, 3H). MS (ESI) m/z. 567.2 [M+H]+.

### Example 114. Synthesis of Compound 115

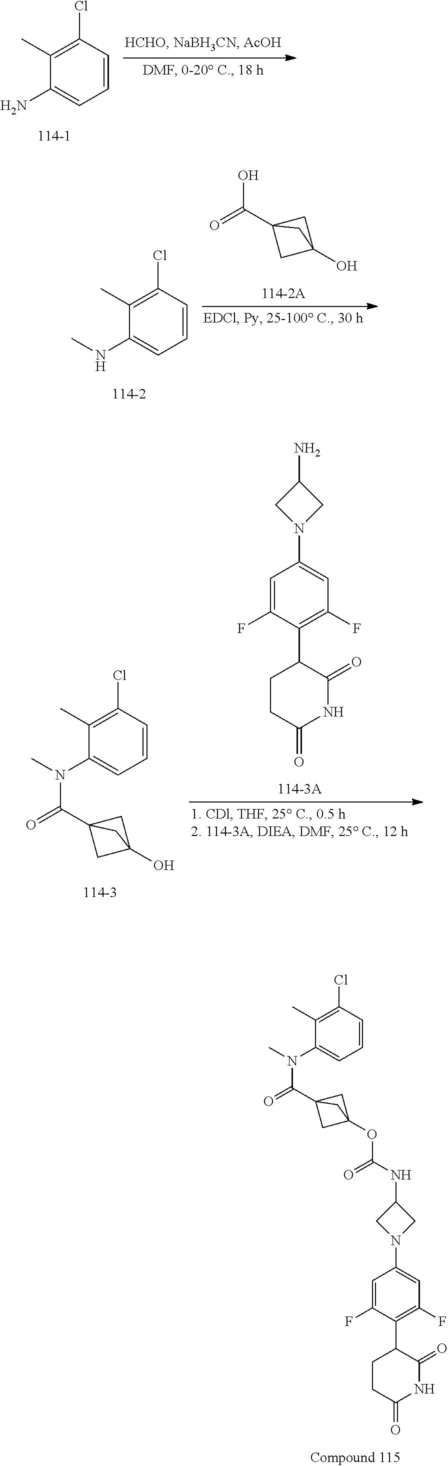

### Step 1. Procedure for Preparation of Compound 2—3-chloro-N, 2-dimethylaniline

To a solution of 3-chloro-2-methylaniline (1.00 g, 7.06 mmol, 840 uL, 1.00 eq) in dimethylformamide (10.0 mL) was added sodium cyanoborohydride (1.33 g, 21.2 mmol, 3.00 eq) and acetic acid (42.4 mg, 706 umol, 40.4 uL, 0.100 eq) and formaldehyde (459 mg, 5.65 mmol, 421 uL, 37.0% purity, 0.800 eq) at 0° C. The mixture was stirred at 20° C. for 18 h. The reaction mixture was diluted with water (20.0 mL) and extracted with ethyl acetate (20 mL*2). The combined organic layers were washed with brine, dried over sodium sulfate, filtered and concentrated under reduced pressure to give a residue. The residue was purified by column chromatography (SiO2, Petroleum ether/Ethyl acetate=1/0 to 10/1) to afford 3-chloro-N, 2-dimethylaniline (170 mg, 1.09 mmol, 15.5% yield) as yellow oil.

1H NMR (400 MHz, DMSO-d6) δ=7.01 (t, J=8.0 Hz, 1H), 6.62 (d, J=7.8 Hz, 1H), 6.43 (d, J=8.0 Hz, 1H), 5.34 (br d, J=4.4 Hz, 1H), 2.72 (d, J=5.0 Hz, 3H), 2.12 (s, 3H).

### Step 2. Procedure for Preparation of Compound 3—N-(3-chloro-2-methylphenyl)-3-hydroxy-N-methylbicyclo[1.1.1]pentane-1-carboxamide

To a solution of 3-hydroxybicyclo[1.1.1]pentane-1-carboxylic acid (100 mg, 780 umol, 1.00 eq) in pyridine (2.00 mL) was added 1-(3-dimethylaminopropyl)-3-ethylcarbodiimide hydrochloride (224 mg, 1.17 mmol, 1.50 eq) and 3-chloro-N, 2-dimethylaniline (146 mg, 937 umol, 1.20 eq). The mixture was stirred at 25° C. for 12 h. Then 1-(3-dimethylaminopropyl)-3-ethylcarbodiimide hydrochloride (224.43 mg, 1.17 mmol, 1.50 eq) was added. The mixture was stirred at 80° C. for 12 h. Then 1-(3-dimethylaminopropyl)-3-ethylcarbodiimide hydrochloride (224 mg, 1.17 mmol, 1.50 eq) was added. The mixture was stirred at 100° C. for 6 h. The reaction mixture was concentrated under reduced pressure to remove pyridine. The residue was diluted with hydrochloric acid (1 M, 5 mL) and extracted with ethyl acetate (5 mL*2). The combined organic layers were dried over sodium sulfate, filtered and concentrated under reduced pressure to give a residue. The residue was purified by column chromatography (SiO2, Petroleum ether/Ethyl acetate=1/1) to afford N-(3-chloro-2-methylphenyl)-3-hydroxy-N-methylbicyclo[1.1.1]pentane-1-carboxamide (40 mg, 151 umol, 19.3% yield) as yellow oil.

1H NMR (400 MHz, DMSO-d6) δ=7.53 (d, J=7.8 Hz, 1H), 7.34-7.30 (m, 1H), 7.28-7.22 (m, 1H), 6.11 (s, 1H), 3.07-3.03 (m, 3H), 2.19 (s, 3H), 1.64-1.55 (m, 3H), 1.53-1.46 (m, 3H).

### Step 3. Procedure for Preparation of 3-((3-chloro-2-methylphenyl)(methyl)carbamoyl)bicyclo[1.1.1]pentan-1-yl (1-(4-(2,6-dioxopiperidin-3-yl)-3,5-difluorophenyl)azetidin-3-yl)carbamate

To a solution of N-(3-chloro-2-methylphenyl)-3-hydroxy-N-methylbicyclo[1.1.1]pentane-1-carboxamide (40.0 mg, 151 umol, 1.00 eq) in tetrahydrofuran (1.00 mL) was added di(1H-imidazol-1-yl)methanone (24.4 mg, 151 umol, 1.00 eq). The mixture was stirred at 25° C. for 0.5 h. The resulting solution was added into a mixture of 3-(4-(3-aminoazetidin-1-yl)-2,6-difluorophenyl)piperidine-2,6-dione (59 mg, 150 umol, 1.00 eq, methanesulfonic acid) and N,N-diisopropylethylamine (58.19 mg, 450.24 umol, 78.42 uL, 3.00 eq) in dimethylformamide (1.00 mL). The reaction mixture was stirred at 25° C. for 12 h. The reaction mixture was concentrated under reduced pressure to give a residue. The residue was purified by reverse phase chromatography (C18, 40 g, condition: water/acetonitrile=100:0 to 0:1, 0.1% formic acid) and lyophilized to afford 3-((3-chloro-2-methylphenyl)(methyl)carbamoyl)bicyclo[1.1.1]pentan-1-yl (1-(4-(2,6-dioxopiperidin-3-yl)-3,5-difluorophenyl)azetidin-3-yl)carbamate (25.77 mg, 40.83 umol, 27.20% yield, 93% purity) as a white solid.

1H NMR (400 MHz, DMSO-d6) δ=10.84 (s, 1H), 7.84 (d, J=7.3 Hz, 1H), 7.57-7.51 (m, 1H), 7.36-7.30 (m, 1H), 7.30-7.26 (m, 1H), 6.12 (d, J=11.3 Hz, 2H), 4.37-4.23 (m, 1H), 4.03 (br t, J=7.1 Hz, 3H), 3.55 (br t, J=6.7 Hz, 2H), 3.06 (s, 3H), 2.80-2.73 (m, 1H), 2.56-2.55 (m, 1H), 2.19 (s, 3H), 2.06 (br d, J=11.5 Hz, 1H), 1.94 (br d, J=1.9 Hz, 1H), 1.89 (d, J=9.8 Hz, 3H), 1.82-1.76 (m, 3H). MS (ESI) m/z. 587.1 [M+H]+.

### Example 115. Synthesis of Compound 116

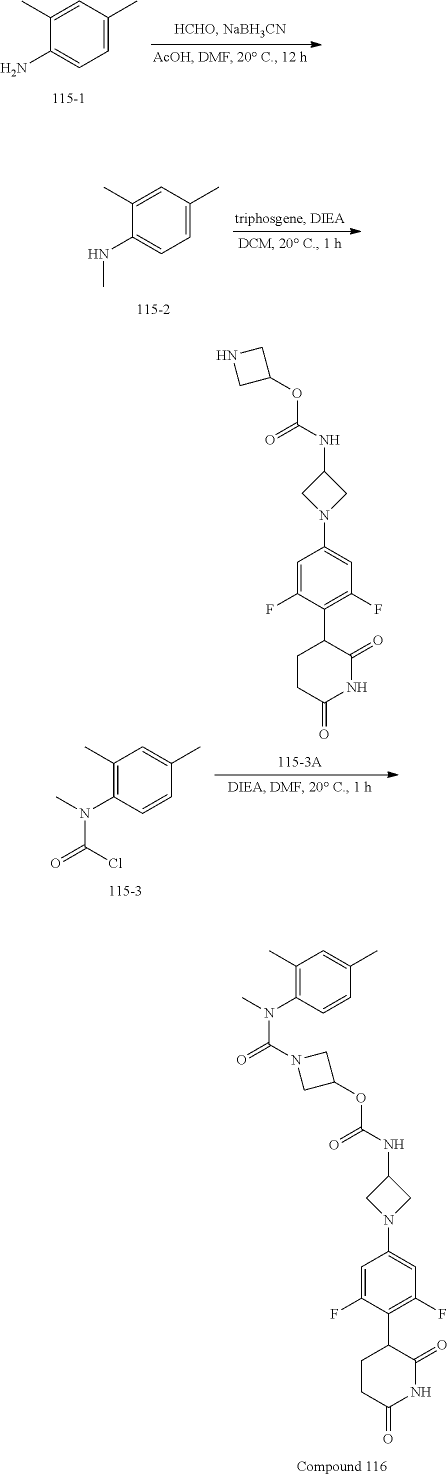

### Step 1. Procedure for Compound 2—N, 2,4-trimethylaniline

To a solution of 2,4-dimethylaniline (3.00 g, 24.8 mmol, 1.00 eq) in N, N-dimethyl formamide (30.0 mL) was added formaldehyde (1.61 g, 19.8 mmol, 1.47 mL, 37% purity, 0.800 eq), sodium cyanoborohydride (3.11 g, 49.5 mmol, 2.00 eq) and acetic acid (149 mg, 2.48 mmol, 142 uL, 0.100 eq). The mixture was stirred at 20° C. for 12 h. The reaction mixture was quenched by addition water (50 mL), and extracted with ethyl acetate (3×50 mL). The combined organic layers were washed with water (2×50 mL), dried over anhydrous sodium sulfate, filtered and concentrated under reduced pressure to give a residue. The residue was purified by column chromatography (SiO2, Petroleum ether/Ethyl acetate=1/0 to 10/1) to afford N, 2,4-trimethylaniline (0.673 g, 4.98 mmol, 20% yield) as yellow oil.

1H NMR (400 MHz, CDCl3-d) δ=6.97 (br d, J=8.1 Hz, 1H), 6.90 (s, 1H), 6.54 (d, J=8.1 Hz, 1H), 2.88 (s, 3H), 2.25 (s, 3H), 2.12 (s, 3H)

### Step 2. Procedure for Compound 3—(2,4-dimethylphenyl)(methyl)carbamic chloride

To a solution of N, 2,4-trimethylaniline (32.0 mg, 237 umol, 1.00 eq) in dichloromethane (1.00 mL) was added N,N-diisopropylethylamine (61.2 mg, 473 umol, 82.5 uL, 2.00 eq) and triphosgene (70.2 mg, 237 umol, 1.00 eq) at 0° C. The reaction mixture was stirred at 20° C. for 1 h. The reaction mixture was concentrated under reduced pressure to afford (2,4-dimethylphenyl)(methyl)carbamic chloride (46.0 mg, 233 umol, 98% yield) as yellow oil.

### Step 3. Procedure for 1-((2,4-dimethylphenyl)(methyl)carbamoyl)azetidin-3-yl (1-(4-(2,6-dioxopiperidin-3-yl)-3,5-difluorophenyl)azetidin-3-yl)carbamate

To a solution of azetidin-3-yl (1-(4-(2,6-dioxopiperidin-3-yl)-3,5-difluorophenyl)azetidin-3-yl)carbamate (79.0 mg, 200 umol, 1.00 eq) in N,N-dimethyl formamide (1.00 mL) was added N,N-diisopropylethylamine (51.8 mg, 400 umol, 69.8 uL, 2.00 eq) and (2,4-dimethylphenyl)(methyl)carbamic chloride (43.6 mg, 220 umol, 1.10 eq). The mixture was stirred at 20° C. for 2 h. The reaction mixture was concentrated under reduced pressure to give a residue. The residue was purified by reversed-phase HPLC(C18, 80 g; condition: water/acetonitrile=1/0 to 0/1, 0.1% formic acid)) and lyophilized to afford 1-((2,4-dimethylphenyl)(methyl)carbamoyl)azetidin-3-yl-(l-(4-(2,6-dioxopiperidin-3-yl)-3,5-difluorophenyl)azetidin-3-yl)carbamate (15.1 mg, 26.64 umol, 13% yield, 98% purity) as a white solid.

1H NMR (400 MHz, DMSO-d6) δ=10.92-10.78 (m, 1H), 8.03 (br d, J=6.5 Hz, 1H), 7.16-7.02 (m, 3H), 6.13 (d, J=11.1 Hz, 2H), 4.83-4.69 (m, 1H), 4.40 (br d, J=6.8 Hz, 1H), 4.11-4.00 (m, 3H), 3.69-3.52 (m, 4H), 3.24 (br dd, J=2.9, 9.5 Hz, 2H), 2.98 (s, 3H), 2.85-2.71 (m, 1H), 2.59-2.54 (m, 1H), 2.28 (s, 3H), 2.14 (s, 3H), 2.12-2.05 (m, 1H), 1.99-1.91 (m, 1H). MS (ESI) m/z 556.3[M+H]+.

### Example 116. Synthesis of Compound 117

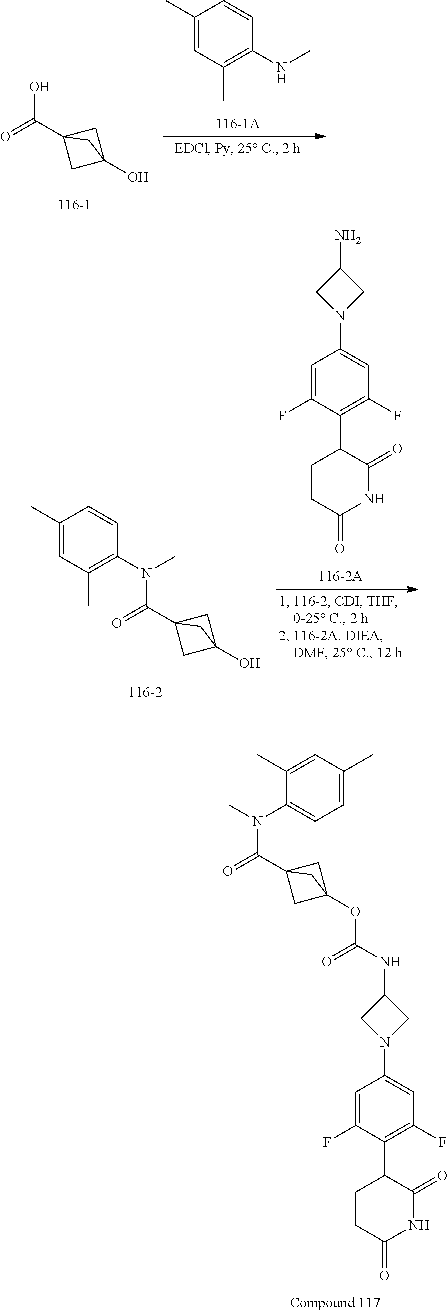

### Step 1. Procedure for Preparation of Compound 2—N-(2,4-dimethylphenyl)-3-hydroxy-N-methylbicyclo[1.1.1]pentane-1-carboxamide

To a solution of 3-hydroxybicyclo[1.1.1]pentane-1-carboxylic acid (100 mg, 780 umol, 1.00 eq) in pyridine (1.00 mL) was added N, 2,4-trimethylaniline (127 mg, 937 umol, 1.20 eq) and 1-(3-dimethylaminopropyl)-3-ethylcarbodiimide hydrochloride (224 mg, 1.17 mmol, 1.50 eq). The mixture was stirred at 25° C. for 2 h. The reaction mixture was concentrated under reduced pressure to give a residue. The residue was purified by column chromatography (SiO2, petroleum ether/ethyl acetate=5/1 to 1/1) to afford N-(2,4-dimethylphenyl)-3-hydroxy-N-methylbicyclo[1.1.1]pentane-1-carboxamide (152 mg, 619 umol, 79% yield) as a white solid. MS (ESI) m/z 246.2 [M+H]+.

### Step 2. Procedure for Preparation of 3-((2,4-dimethylphenyl)(methyl)carbamoyl)bicyclo[1.1.1]pentan-1-yl (1-(4-(2,6-dioxopiperidin-3-yl)-3,5-difluorophenyl)azetidin-3-yl)carbamate

To the solution of N-(2,4-dimethylphenyl)-3-hydroxy-N-methylbicyclo[1.1.1]pentane-1-carboxamide (50.0 mg, 203 umol, 1.00 eq) in tetrahydrofuran (0.500 mL) was added di(1H-imidazol-1-yl)methanone (56.1 mg, 346 umol, 1.70 eq) at 0° C. Then the reaction was stirred at 25° C. for 2 h. Then the mixture was added into a solution of N,N-diisopropylethylamine (39.4 mg, 304 umol, 53.1 uL, 1.50 eq) and 3-(4-(3-aminoazetidin-1-yl)-2,6-difluorophenyl)piperidine-2,6-dione (79.5 mg, 203 umol, 1.00 eq, mesylate) in N,N-dimethyl formamide (0.500 mL). The mixture was stirred at 25° C. for 12 h. The reaction mixture was concentrated under reduced pressure to give a residue. The residue was purified by Prep-HPLC (column: Phenomenex luna C18 150*25 mm*10 um; mobile phase: [water (formic acid)-acetonitrile]; B %: 40%-70%, 10 min) and lyophilized to afford 3-((2,4-dimethylphenyl)(methyl)carbamoyl)bicyclo[1.1.1]pentan-1-yl (1-(4-(2,6-dioxopiperidin-3-yl)-3,5-difluorophenyl)azetidin-3-yl)carbamate (32.49 mg, 56.77 umol, 27% yield, 99% purity) as a white solid.

1H NMR (400 MHz, DMSO-d6) δ=10.85 (s, 1H), 7.82 (d, J=7.6 Hz, 1H), 7.15 (s, 1H), 7.08 (d, J=1.0 Hz, 2H), 6.12 (d, J=11.0 Hz, 2H), 4.34-4.26 (m, 1H), 4.02 (br t, J=7.2 Hz, 3H), 3.55 (br t, J=6.8 Hz, 2H), 3.02 (s, 3H), 2.82-2.71 (m, 1H), 2.35-2.29 (m, 4H), 2.12 (s, 3H), 2.09-2.00 (m, 1H), 1.97-1.90 (m, 1H), 1.89-1.84 (m, 3H), 1.83-1.76 (m, 3H). MS (ESI) m/z 567.1 [M+H]+.

### Example 117. Synthesis of Compound 118

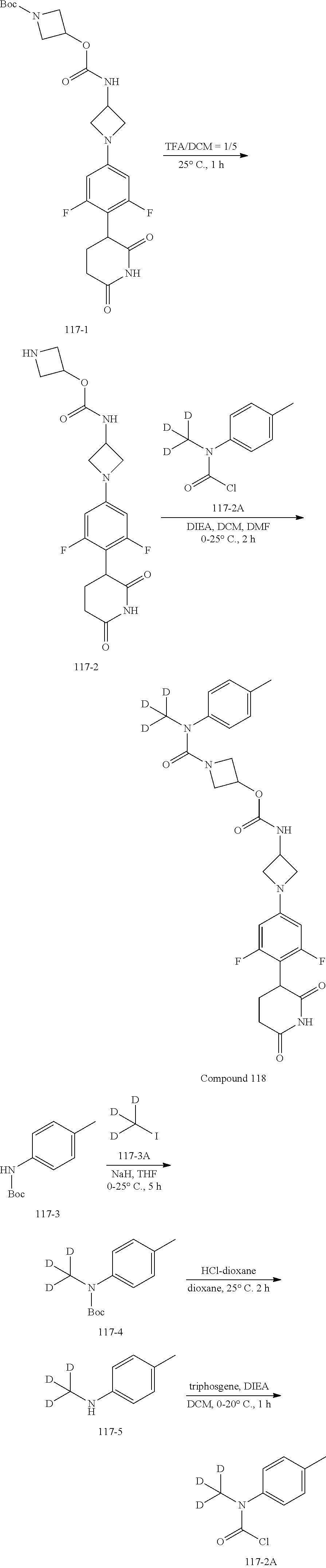

### Step 1. Procedure for Preparation of Compound 4—tert-butyl (methyl-d3)(p-tolyl)carbamate

To a solution of tert-butyl p-tolylcarbamate (500 mg, 2.41 mmol, 1.00 eq) in tetrahydrofuran (5.00 mL) was added sodium hydride (144 mg, 3.62 mmol, 60% purity, 1.50 eq) at 0° C. The mixture was stirred at 0° C. for 1 h. Then iodomethane-d3 (384 mg, 2.65 mmol, 165 uL, 1.10 eq) was added into the mixture. The mixture was stirred at 25° C. for 2 h. Iodomethane-d3 (174 mg, 1.21 mmol, 75.0 uL, 0.500 eq) was added into the mixture. The mixture was stirred at 25° C. for 2 h. The mixture was quenched with saturated ammonium chloride (20 mL), extracted with ethyl acetate (3×20 mL), washed with brine (50 mL), and dried over anhydrous sodium sulfate, filtered and concentrated under reduced pressure to afford tert-butyl (methyl-d3)(p-tolyl)carbamate (600 mg, 2.35 mmol, 97% yield, 88% purity) as yellow oil. ¹H NMR (400 MHz, DMSO-d6) δ=7.13 (s, 4H), 2.27 (s, 3H), 1.37 (s, 9H).

### Step 2. Procedure for Preparation of Compound 5—4-methyl-N-(methyl-d3)aniline

A solution of tert-butyl (methyl-d3)(p-tolyl)carbamate (600 mg, 2.35 mmol, 88% purity, 1.00 eq) in hydrochloric acid (4 M in dioxane) (3.00 mL) and dioxane (3.00 mL) was stirred at 25° C. for 2 h. The mixture was concentrated under reduced pressure to afford 4-methyl-N-(methyl-d3)aniline (400 mg, 2.29 mmol, 97% yield, 92% purity, hydrochloric acid) as a yellow solid. ¹H NMR (400 MHz, DMSO-d6) δ=11.26-10.81 (m, 1H), 7.41-7.35 (m, 2H), 7.34-7.26 (m, 2H), 2.32 (s, 3H).

### Step 3. Procedure for Preparation of Compound 2A—(methyl-d3)(p-tolyl)carbamic chloride

To a solution of 4-methyl-N-(trideuteriomethyl)aniline (50.0 mg, 311 umol, 1.00 eq, hydrochloric acid) in dichloromethane (1.00 mL) was added N,N-diisopropylethylamine (80.4 mg, 622 umol, 108 uL, 2.00 eq) and bis(trichloromethyl) carbonate (92.3 mg, 311 umol, 1.00 eq) at 0° C. The mixture was stirred at 25° C. for 1 h. The mixture was concentrated under reduced pressure to afford (methyl-d3)(p-tolyl)carbamic chloride (60.0 mg, crude, hydrochloric acid) as yellow oil.

### Step 4. Procedure for Preparation of Compound 2—azetidin-3-yl (1-(4-(2,6-dioxopiperidin-3-yl)-3,5-difluorophenyl)azetidin-3-yl)carbamate

A solution of tert-butyl 3-(((1-(4-(2,6-dioxopiperidin-3-yl)-3,5-difluorophenyl)azetidin-3-yl)carbamoyl)oxy)azetidine-1-carboxylate (100 mg, 202 umol, 1.00 eq) in dichloromethane (1.00 mL) and trifluoroacetic acid (0.200 mL). The mixture was stirred at 25° C. for 1 h. The mixture was concentrated under reduced pressure to afford azetidin-3-yl (1-(4-(2,6-dioxopiperidin-3-yl)-3,5-difluorophenyl)azetidin-3-yl)carbamate (79.0 mg, crude) as yellow oil. MS (ESI) m/z 395.3 [M+H]+.

### Step 5. Procedure for Preparation of 1-((methyl-d3)(p-tolyl)carbamoyl)azetidin-3-yl (1-(4-(2,6-dioxopiperidin-3-yl)-3,5-difluorophenyl)azetidin-3-yl)carbamate

To a solution of afford azetidin-3-yl (1-(4-(2,6-dioxopiperidin-3-yl)-3,5-difluorophenyl)azetidin-3-yl)carbamate (79.0 mg, 200 umol, 1.00 eq) in dichloromethane (1.00 mL) was added N,N-diisopropylethylamine (129 mg, 1.00 mmol, 174 uL, 5.00 eq). A solution of (methyl-d3)(p-tolyl)carbamic chloride (49.1 mg, 220 umol, 1.10 eq, hydrochloric acid) in dichloromethane (1.00 mL) was added into the mixture at 0° C. The mixture was stirred at 0° C. for 1 h. Dimethylformamide (1.00 mL) was added into the mixture. The mixture was stirred at 25° C. for 1 h. The mixture was concentrated under reduced pressure to give a residue. The residue was purified by reversed-phase HPLC (0.1% formic acid condition) and lyophilized to afford 1-((methyl-d3)(p-tolyl)carbamoyl)azetidin-3-yl (1-(4-(2,6-dioxopiperidin-3-yl)-3,5-difluorophenyl)azetidin-3-yl)carbamate (17.4 mg, 29.40 umol, 14% yield, 92% purity) as a white solid. 1H NMR (400 MHz, DMSO-d6) δ=10.85 (s, 1H), 8.03 (br d, J=7.2 Hz, 1H), 7.22-7.17 (m, 2H), 7.16-7.10 (m, 2H), 6.13 (br d, J=11.2 Hz, 2H), 4.83-4.74 (m, 1H), 4.41-4.30 (m, 1H), 4.09-4.03 (m, 2H), 4.01 (br d, J=4.8 Hz, 1H), 3.76-3.66 (m, 2H), 3.59 (br t, J=6.8 Hz, 2H), 3.31-3.28 (m, 2H), 2.83-2.71 (m, 1H), 2.48-2.47 (m, 1H), 2.30 (s, 3H), 2.06 (br dd, J=4.4, 12.0 Hz, 1H), 1.98-1.88 (m, 1H). MS (ESI) m/z 545.3 [M+H]+.

### Example 118. Synthesis of Compound 119

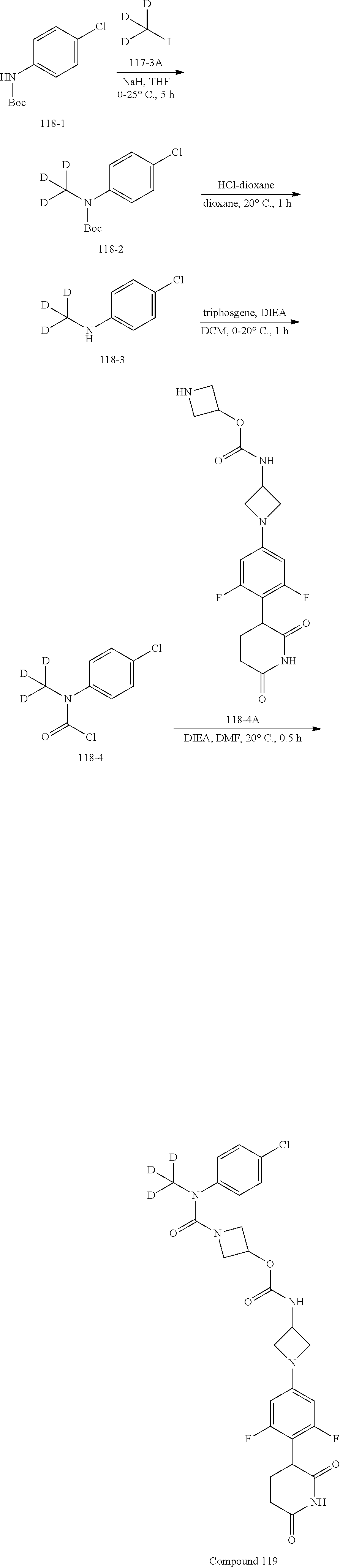

### Step 1. Procedure for Compound 2—tert-butyl (4-chlorophenyl)(methyl-d3)carbamate

To a solution of tert-butyl (4-chlorophenyl)carbamate (0.500 g, 2.20 mmol, 1.00 eq) in tetrahydrofuran (10.0 mL) was added sodium hydride (96.6 mg, 2.42 mmol, 60% purity, 1.10 eq) at 0° C. The reaction mixture was stirred at 0° C. for 0.5 h. Then iodomethane-d3 (350 mg, 2.42 mmol, 150 uL, 1.10 eq) was added. The reaction was stirred at 20° C. for 11.5 h. The reaction mixture was quenched with water (20.0 mL) and extracted with ethyl acetate (3×50.0 mL). The organic layer was washed with brine (50.0 mL), dried over anhydrous sodium sulfate, and concentrated under reduced pressure to afford tert-butyl (4-chlorophenyl)(methyl-d3)carbamate (512 mg, 2.09 mmol, 95% yield) as a yellow oil.

¹H NMR (400 MHz, CDCl3-d) δ=7.29-7.25 (m, 2H), 7.17 (d, J=8.8 Hz, 2H), 1.45 (s, 9H).

### Step 2. Procedure for Compound 3—4-chloro-N-(methyl-d3)aniline

To a solution of tert-butyl (4-chlorophenyl)(methyl-d3)carbamate (500 mg, 2.04 mmol, 1.00 eq) in dioxane (5.00 mL) was added 4 N of hydrochloric acid in dioxane (5.00 mL). The reaction mixture was stirred at 20° C. for 1 h. The reaction mixture was concentrated under reduced pressure to afford 4-chloro-N-(methyl-d3)aniline (295 mg, 1.63 mmol, 79.74% yield, hydrochloric acid) as a yellow oil.

¹H NMR (400 MHz, CDCl3-d) δ=7.42 (d, J=8.8 Hz, 2H), 7.21 (br d, J=8.4 Hz, 2H).

### Step 3. Procedure for Compound 4—(4-chlorophenyl)(methyl-d3)carbamic chloride

To a solution of 4-chloro-N-(methyl-d3)aniline (50.0 mg, 276 umol, 1.00 eq, hydrochloric acid) in dichloromethane (1.00 mL) was added N,N-diisopropylethylamine (71.4 mg, 552 umol, 96.2 uL, 2.00 eq) and triphosgene (81.9 mg, 276 umol, 1.00 eq) at 0° C. The reaction mixture was stirred at 20° C. for 1 h. The reaction mixture was concentrated under reduced pressure to afford (4-chlorophenyl)(methyl-d3)carbamic chloride (57.0 mg, 275 umol, 99% yield) as a yellow oil.

### Step 4. Procedure for 1-((4-chlorophenyl)(methyl-d3)carbamoyl)azetidin-3-yl (1-(4-(2,6-dioxopiperidin-3-yl)-3,5-difluorophenyl)azetidin-3-yl)carbamate

To a solution of azetidin-3-yl (1-(4-(2,6-dioxopiperidin-3-yl)-3,5-difluorophenyl)azetidin-3-yl)carbamate (79.0 mg, 200 umol, 1.00 eq) in dimethyformamide (1.00 mL) was added N,N-diisopropylethylamine (51.8 mg, 400 umol, 69.8 uL, 2.00 eq) and (4-chlorophenyl)(methyl-d3)carbamic chloride (45.6 mg, 220 umol, 1.10 eq). The reaction mixture was stirred at 20° C. for 0.5 h. The reaction mixture was filtered. The filtrate was purified by Prep-HPLC (column: Phenomenex luna C18 150*25 mm*10 um; mobile phase: [water(formic acid)-acetonitrile]; B %: 42%-72%, 7 min) and lyophilized to afford 1-((4-chlorophenyl)(methyl-d3)carbamoyl)azetidin-3-yl-(1-(4-(2,6-dioxopiperidin-3-yl)-3,5-difluorophenyl)azetidin-3-yl)carbamate (28.79 mg, 47.39 umol, 23.66% yield, 93% purity) as a yellow solid.

1H NMR (400 MHz, DMSO-d6) δ=10.8 (s, 1H), 8.04 (br d, J=7.5 Hz, 1H), 7.51-7.38 (m, 2H), 7.33-7.26 (m, 2H), 6.13 (br d, J=11.1 Hz, 2H), 4.90-4.73 (m, 1H), 4.41-4.29 (m, 1H), 4.10-3.97 (m, 3H), 3.83-3.74 (m, 2H), 3.59 (br t, J=6.6 Hz, 2H), 3.40-3.36 (m, 2H), 2.80-2.72 (m, 1H), 2.39 (br d, J=3.8 Hz, 1H), 2.08-2.01 (m, 1H), 1.98-1.90 (m, 1H). MS (ESI) m/z. 565.3 [M+H]+.

### Example 119. Synthesis of Compound 120

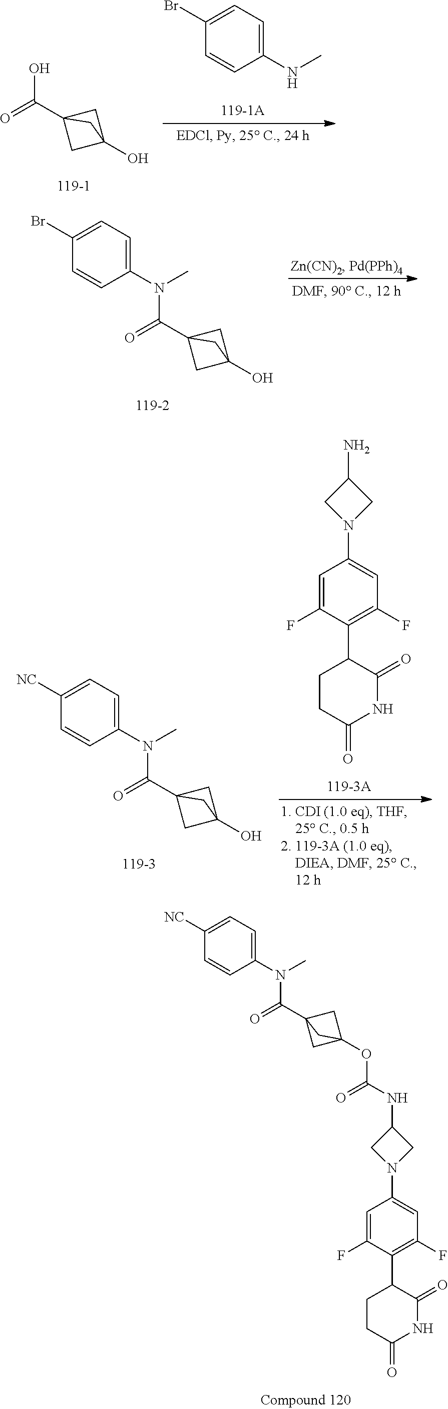

### Step 1. Procedure for Preparation of Compound 2—N-(4-bromophenyl)-3-hydroxy-N-methylbicyclo[1.1.1]pentane-1-carboxamide

To a solution of 3-hydroxybicyclo[1.1.1]pentane-1-carboxylic acid (200 mg, 1.56 mmol, 1.00 eq) in pyridine (4.00 mL) was added 1-(3-dimethylaminopropyl)-3-ethylcarbodiimide hydrochloride (449 mg, 2.34 mmol, 1.50 eq) and 4-bromo-N-methyl-aniline (436 mg, 2.34 mmol, 1.50 eq). The mixture was stirred at 25° C. for 12 h. The reaction mixture was concentrated under reduced pressure to remove pyridine. The residue was diluted with ethyl acetate (10 mL). The combined organic layers were washed with hydrochloric acid (1M, 5 mL), dried over sodium sulfate, filtered and concentrated under reduced pressure to give a residue. The residue was purified by column chromatography (SiO2, dichloromethane/methanol=6/1) to afford N-(4-bromophenyl)-3-hydroxy-N-methylbicyclo[1.1.1]pentane-1-carboxamide (373 mg, 1.26 mmol, 80.7% yield) as a purple solid.

¹H NMR (400 MHz, DMSO-d6) δ=7.65 (d, J=8.5 Hz, 2H), 7.36-7.22 (m, 2H), 6.13 (br s, 1H), 3.10 (s, 3H), 1.60 (br s, 6H).

### Step 2. Procedure for Preparation of Compound 3—N-(4-cyanophenyl)-3-hydroxy-N-methylbicyclo[1.1.1]pentane-1-carboxamide

A mixture of N-(4-cyanophenyl)-3-hydroxy-N-methylbicyclo[1.1.1]pentane-1-carboxamide (370 mg, 1.25 mmol, 1.00 eq), cyanide zinc (220.05 mg, 1.87 mmol, 118.95 uL, 1.5 eq), palladium triphenylphosphane (144 mg, 125 umol, 0.100 eq) in dimethylformamide (4.00 mL) was degassed and purged with nitrogen for 3 times, and then the mixture was stirred at 90° C. for 12 h under nitrogen atmosphere. The reaction mixture was quenched by addition water (20 mL) at 25° C. and extracted with ethyl acetate (20 mL*2). The combined organic layers were dried over sodium sulfate, filtered and concentrated under reduced pressure to give a residue. The residue was purified by column chromatography (SiO2, dichloromethane/methanol=1/0 to 0/1) to afford N-(4-cyanophenyl)-3-hydroxy-N-methylbicyclo[1.1.1]pentane-1-carboxamide (125 mg, 516 umol, 41.3% yield) as colorless oil.

¹H NMR (400 MHz, DMSO-d6) δ=7.95 (d, J=2.4 Hz, 2H), 7.59-7.46 (m, 2H), 6.16 (s, 1H), 3.16 (s, 3H), 1.66 (s, 6H).

### Step 3. Procedure for Preparation of 3-((4-cyanophenyl)(methyl)carbamoyl)bicyclo[1.1.1]pentan-1-yl (1-(4-(2,6-dioxopiperidin-3-yl)-3,5-difluorophenyl)azetidin-3-yl)carbamate

To a solution of N-(4-cyanophenyl)-3-hydroxy-N-methylbicyclo[1.1.1]pentane-1-carboxamide (90.0 mg, 371 umol, 1.00 eq) in tetrahydrofuran (2.00 mL) was added di(1H-imidazol-1-yl)methanone (60 mg, 371 umol, 1.00 eq). The mixture was stirred at 25° C. for 0.5 h. The resulting solution was added into a mixture of 3-(4-(3-aminoazetidin-1-yl)-2,6-difluorophenyl)piperidine-2,6-dione (144 mg, 369 umol, 1.00 eq, methanesulfonic acid) and N,N-diisopropylethylamine (143 mg, 1.11 mmol, 193 uL, 3.00 eq) in dimethylformamide (2.00 mL). The reaction mixture was stirred at 25° C. for 12 h. The reaction mixture was concentrated under reduced pressure to give a residue. The residue was purified by reverse phase chromatography (C18, 40 g, condition: water/acetonitrile=100:0 to 0:1, 0.1% formic acid) and lyophilized to afford 3-((4-cyanophenyl)(methyl)carbamoyl)bicyclo[1.1.1]pentan-1-yl (1-(4-(2,6-dioxopiperidin-3-yl)-3,5-difluorophenyl)azetidin-3-yl)carbamate (43.49 mg, 75.63 umol, 20.51% yield, 98% purity) as a white solid.

1H NMR (400 MHz, DMSO-d6) δ=10.84 (s, 1H), 7.94 (d, J=8.5 Hz, 2H), 7.90-7.80 (m, 1H), 7.56 (d, J=8.5 Hz, 2H), 6.11 (br d, J=11.0 Hz, 2H), 4.42-4.23 (m, 1H), 4.07-3.96 (m, 3H), 3.55 (br t, J=6.8 Hz, 2H), 3.16 (s, 3H), 2.78-2.72 (m, 1H), 2.61-2.56 (m, 1H), 2.14-2.05 (m, 1H), 2.02 (br dd, J=3.9, 13.0 Hz, 1H), 1.94 (s, 6H). MS (ESI) m/z. 564.3 [M+H]+.

### Example 120. Synthesis of Compound 121

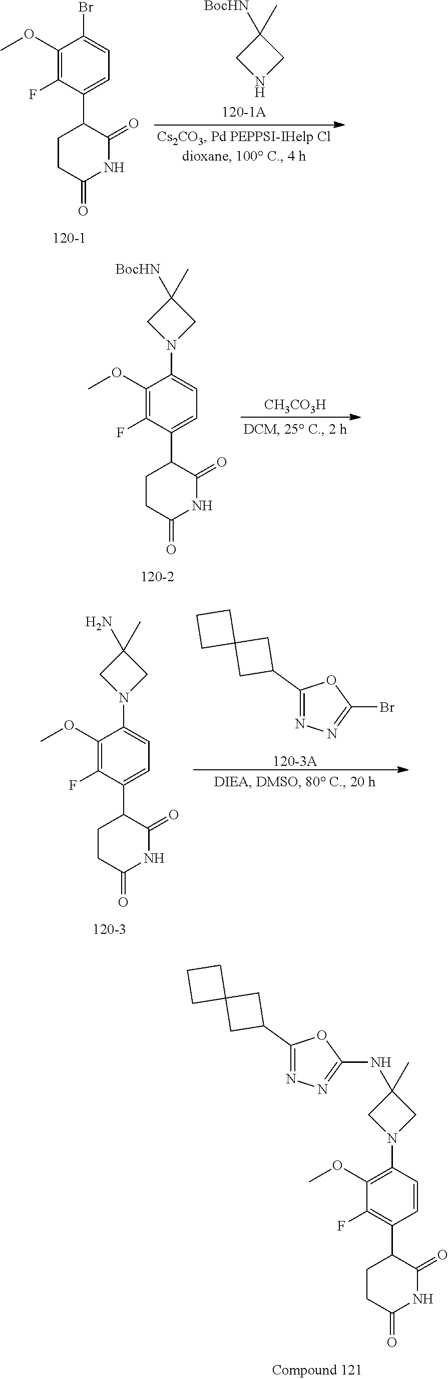

### Step 1. Procedure for Preparation of Compound 2—tert-butyl (1-(4-(2,6-dioxopiperidin-3-yl)-3-fluoro-2-methoxyphenyl)-3-methylazetidin-3-yl)carbamate

To a solution of tert-butyl (3-methylazetidin-3-yl)carbamate (220 mg, 987 umol, 1.20 eq, concentrated hydrochloric acid) in dioxane (5.00 mL) was added 3-(4-bromo-2-fluoro-3-methoxyphenyl)piperidine-2,6-dione (260 mg, 822 umol, 1.00 eq) and cesium carbonate (938 mg, 2.88 mmol, 3.50 eq) and 1,3-bis[2,6-bis(1-propylbutyl)phenyl]-4,5-dichloro-2H-imidazol-1-ium-2-ide;3chloropyridine;dichloropalladium (80.0 mg, 82.3 umol, 0.100 eq). The mixture was stirred at 100° C. for 4 h under nitrogen atmosphere. The reaction mixture was filtered and concentrated under reduced pressure to give a residue. The residue was purified by reverse phase chromatography (C18, 40 g, condition: water/acetonitrile=100:0 to 0:1, 0.1% formic) and lyophilized to afford tert-butyl (1-(4-(2,6-dioxopiperidin-3-yl)-3-fluoro-2-methoxyphenyl)-3-methylazetidin-3-yl)carbamate (180 mg, 427 umol, 51.9% yield) as yellow oil.

1H NMR (400 MHz, DMSO-d6) δ=10.80 (s, 1H), 7.33 (ddd, J=1.9, 3.8, 17.2 Hz, 1H), 6.84-6.74 (m, 1H), 6.21 (d, J=8.5 Hz, 1H), 3.93-3.82 (m, 3H), 3.78-3.72 (m, 2H), 3.68 (s, 3H), 2.77-2.70 (m, 1H), 2.61-2.56 (m, 1H), 2.20-2.08 (m, 1H), 2.01-1.94 (m, 1H), 1.51 (s, 3H), 1.39 (s, 9H)

### Step 2. Procedure for Preparation of Compound 3—3-[4-(3-amino-3-methyl-azetidin-1-yl)-2-fluoro-3-methoxy-phenyl]piperidine-2,6-dione

To a solution of tert-butyl (1-(4-(2,6-dioxopiperidin-3-yl)-3-fluoro-2-methoxyphenyl)-3-methylazetidin-3-yl)carbamate (180 mg, 427 umol, 1.00 eq) in dichloromethane (1.50 mL) was added methanesulfonic acid (123 mg, 1.28 mmol, 91.2 uL, 3.00 eq). The mixture was stirred at 25° C. for 2 h. The reaction mixture was concentrated under reduced pressure to afford 3-[4-(3-amino-3-methyl-azetidin-1-yl)-2-fluoro-3-methoxy-phenyl]piperidine-2,6-dione (137 mg, crude) as yellow oil.

### Step 3. Procedure for Preparation of 3-(2-fluoro-3-methoxy-4-(3-methyl-3-((5-(spiro[3.3]heptan-2-yl)-1,3,4-oxadiazol-2-yl)amino)azetidin-1-yl)phenyl)piperidine-2,6-dione

To a solution of 3-[4-(3-amino-3-methyl-azetidin-1-yl)-2-fluoro-3-methoxy-phenyl]piperidine-2,6-dione (137 mg, 426 umol, 1.00 eq) in dimethylsulfoxide (2.00 mL) was added N,N-diisopropylethylamine (276 mg, 2.13 mmol, 371 uL, 5.00 eq) and 2-bromo-5-spiro[3.3]heptan-2-yl-1,3,4-oxadiazole (104 mg, 426 umol, 1.00 eq). The mixture was stirred at 80° C. for 20 h. The reaction mixture was concentrated under reduced pressure to give a residue. The residue was purified by reverse phase chromatography (C18, 40 g, condition: water/acetonitrile=100:0 to 0:1, 0.1% formic) and lyophilized to afford 3-(2-fluoro-3-methoxy-4-(3-methyl-3-((5-(spiro[3.3]heptan-2-yl)-1,3,4-oxadiazol-2-yl)amino)azetidin-1-yl)phenyl)piperidine-2,6-dione (18.81 mg, 36.57 umol, 8.58% yield, 94% purity) as a white solid.

1H NMR (400 MHz, DMSO-d6) δ=10.80 (br s, 1H), 7.98 (s, 1H), 6.80 (br t, J=8.0 Hz, 1H), 6.24 (br d, J=8.6 Hz, 1H), 4.00 (br d, J=7.8 Hz, 2H), 3.91-3.86 (m, 1H), 3.83 (br d, J=7.9 Hz, 2H), 3.69 (s, 3H), 3.42 (br s, 1H), 2.71-2.65 (m, 1H), 2.60 (br s, 1H), 2.37-2.32 (m, 2H), 2.23-2.13 (m, 3H), 2.09-2.02 (m, 3H), 1.93-1.88 (m, 2H), 1.81-1.74 (m, 2H), 1.62 (s, 3H). MS (ESI) m/z. 484.2 [M+H]+.

### Example 121. Synthesis of Compound 122

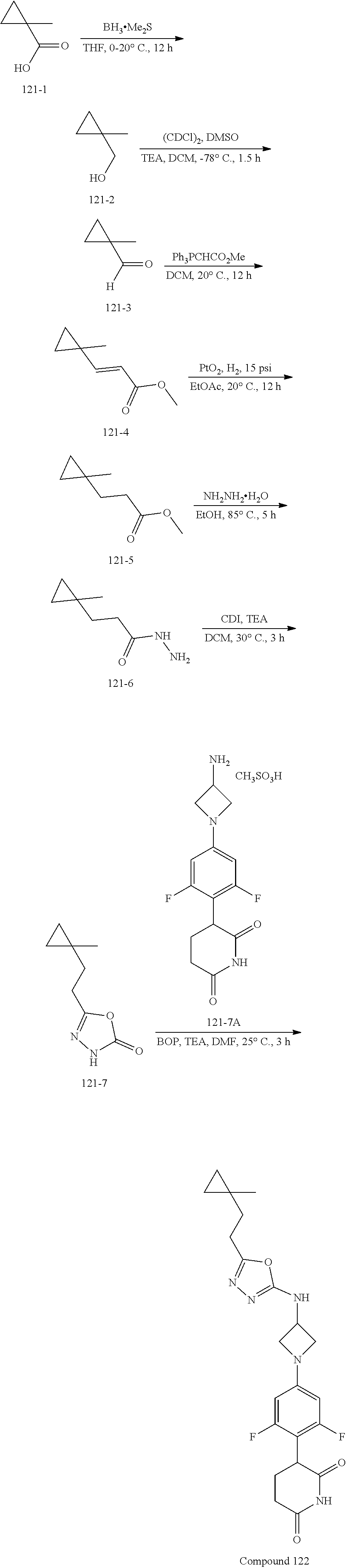

### Step 1. Procedure for Compound 2—(1-methylcyclopropyl)methanol

To a solution of 1-methylcyclopropane-1-carboxylic acid (24.0 g, 240 mmol, 1.00 eq) in tetrahydrofuran (240 mL) was added borane dimethyl sulfide complex (10 M, 36.0 mL, 1.50 eq) at 0° C. The reaction mixture was stirred at 20° C. for 12 h. The reaction mixture was quenched with methanol (120 mL) slowly and concentrated under reduced pressure to give (1-methylcyclopropyl)methanol (12.0 g, 139 mmol, 58% yield) as a colorless oil.

1H NMR (400 MHz, CDCl3-d) δ=3.68-3.48 (m, 1H), 3.38 (s, 2H), 1.15 (s, 3H), 0.42-0.37 (m, 2H), 0.36-0.29 (m, 2H).

### Step 2. Procedure for Compound 3—1-methylcyclopropane-1-carbaldehyde

To a solution of oxalyl dichloride (31.5 g, 248 mmol, 21.8 mL, 2.00 eq) in dichloromethane (48.0 mL) was added dimethylsulfoxide (38.8 g, 497 mmol, 38.8 mL, 4.00 eq) at −78° C. Then the reaction was stirred at −78° C. for 0.5 h. Then a solution of (1-methylcyclopropyl)methanol (10.7 g, 124 mmol, 1.00 eq) in dichloromethane (120 mL) was added at −78° C. The reaction mixture was stirred at −78° C. for 0.5 h. Then triethylamine (101 g, 994 mmol, 138 mL, 8.00 eq) was added at −78° C. The reaction mixture was stirred at −78° C. for 0.5 h. The reaction mixture was quenched with water (200 mL) and extracted with dichloromethane (3×100 mL). The organic layer was washed with hydrochloric acid (1 M, 100 mL), brine (100 mL), dried over sodium sulfate filtered and concentrated under reduced pressure to afford 1-methylcyclopropane-1-carbaldehyde (14.0 g, crude) as a yellow oil.

1H NMR (400 MHz, CDCl3-d) δ=8.64 (s, 1H), 1.25 (s, 3H), 1.18-1.15 (m, 2H), 0.96-0.90 (m, 2H).

### Step 3. Procedure for Compound 4—methyl (E)-3-(1-methylcyclopropyl)acrylate

Then a solution of 1-methylcyclopropane-1-carbaldehyde (10.0 g, 119 mmol, 1.13 mL, 1.00 eq) in dichloromethane (100 mL) was added methyl 2-(triphenyl-λ5-phosphanylidene)acetate (39.8 g, 119 mmol, 1.00 eq). The reaction mixture was stirred at 20° C. for 12 h. The reaction mixture was concentrated under reduced pressure. The residue was purified by chromatography on silica gel (petroleum ether/ethyl acetate=1/0 to 10/1) to afford methyl (E)-3-(1-methylcyclopropyl)acrylate (2.50 g, 17.8 mmol, 15% yield) as a colorless oil

1H NMR (400 MHz, CDCl3-d) δ=6.52 (d, J=15.7 Hz, 1H), 5.77 (d, J=15.7 Hz, 1H), 3.72 (s, 3H), 1.22 (s, 3H), 0.89-0.75 (m, 4H).

### Step 4. Procedure for Compound 5—methyl 3-(1-methylcyclopropyl)propanoate

Then a solution of methyl (E)-3-(1-methylcyclopropyl)acrylate (1.00 g, 7.13 mmol, 1.00 eq) in ethyl acetate (15.0 mL) was added platinum(IV) dioxide (648 mg, 2.85 mmol, 0.400 eq). The reaction mixture was stirred at 20° C. for 12 h under 15 psi of hydrogen atmosphere. The reaction mixture was filtered and concentrated under reduced pressure to afford methyl 3-(1-methylcyclopropyl)propanoate (0.500 g, 3.52 mmol, 49% yield) as a colorless oil.

1H NMR (400 MHz, CDCl3-d) δ=3.67 (s, 3H), 2.44-2.36 (m, 2H), 1.61-1.53 (m, 2H), 1.02 (s, 3H), 0.31-0.19 (m, 4H).

### Step 5. Procedure for Compound 6—3-(1-methylcyclopropyl)propanehydrazide

Then a solution of methyl 3-(1-methylcyclopropyl)propanoate (1.30 g, 9.14 mmol, 1.00 eq) in ethanol (20.0 mL) was added hydrazine hydrate (5.38 g, 91.4 mmol, 5.23 mL, 85% purity, 10.0 eq). The reaction mixture was stirred at 80° C. for 3 h. The reaction mixture was concentrated under reduced pressure to afford crude product. The crude product was purified by reverse phase chromatography (C18, 80 g; condition: water/acetonitrile=1/0 to 0/1, 0.1% formic acid) and lyophilized to afford 3-(1-methylcyclopropyl)propanehydrazide (330 mg, 2.32 mmol, 25% yield) as a white solid. 1H NMR (400 MHz, DMSO-d6) δ=8.92 (br s, 1H), 4.11 (s, 2H), 2.10-2.02 (m, 2H), 1.47-1.36 (m, 2H), 0.97 (s, 3H), 0.26-0.21 (m, 2H), 0.20-0.15 (m, 2H).

### Step 6. Procedure for Compound 7—5-(2-(1-methylcyclopropyl)ethyl)-1,3,4-oxadiazol-2(3H)-one

Then a solution of 3-(1-methylcyclopropyl)propanehydrazide (17.0 mg, 120 umol, 1.00 eq) in dichloromethane (1.00 mL) was added triethylamine (36.3 mg, 359 umol, 50.0 uL, 3.00 eq) and 1,1′-carbonyldiimidazole (38.8 mg, 239 umol, 2.00 eq). The reaction mixture was stirred at 30° C. for 3 h. The reaction mixture was concentrated under reduced pressure to afford 5-(2-(1-methylcyclopropyl)ethyl)-1,3,4-oxadiazol-2(3H)-one (20.0 mg, 119 umol, 99% yield) as a white solid.

### Step 7. Procedure for 3-(2,6-difluoro-4-(3-((5-(2-(1-methylcyclopropyl)ethyl)-1,3,4-oxadiazol-2-yl)amino)azetidin-1-yl)phenyl)piperidine-2,6-dione

Then a solution of 5-(2-(1-methylcyclopropyl)ethyl)-1,3,4-oxadiazol-2(3H)-one (20.0 mg, 119 umol, 1.00 eq) in dimethyformamide (1.00 mL) was added 3-(4-(3-aminoazetidin-1-yl)-2,6-difluorophenyl) piperidine-2,6-dione (46.5 mg, 119 umol, 1.00 eq, methanesulfonic acid), triethylamine (36.1 mg, 357 umol, 49.7 uL, 3.00 eq) and ((1H-benzo[d][1,2,3]triazol-1-yl)oxy)tris(dimethylamino)phosphonium hexafluorophosphate(V) (52.6 mg, 119 umol, 1.00 eq). The reaction mixture was stirred at 25° C. for 3 h. The reaction mixture was filtered. The filtrate was purified by Prep-HPLC (column: Phenomenex C18 75*30 mm*3 um; mobile phase: [water(formic acid)-acetonitrile]; B %: 30%-60%, 7 min) and lyophilized to afford 3-(2,6-difluoro-4-(3-((5-(2-(1-methylcyclopropyl)ethyl)-1,3,4-oxadiazol-2-yl)amino) azetidin-1-yl) phenyl)piperidine-2,6-dione (13.41 mg, 29.8 umol, 25% yield, 99% purity) as a obtained as white solid.

1H NMR (400 MHz, DMSO-d6) δ=10.86 (s, 1H), 8.09 (d, J=7.1 Hz, 1H), 6.19 (d, J=11.0 Hz, 2H), 4.51-4.35 (m, 1H), 4.17 (t, J=7.6 Hz, 2H), 4.10-4.00 (m, 1H), 3.79-3.63 (m, 2H), 2.82-2.69 (m, 3H), 2.11-1.87 (m, 3H), 1.60-1.52 (m, 2H), 1.02 (s, 3H), 0.27-0.18 (m, 4H). MS (ESI) m/z. 446.2 [M+H]+.

### Example 122. Synthesis of Compound 123

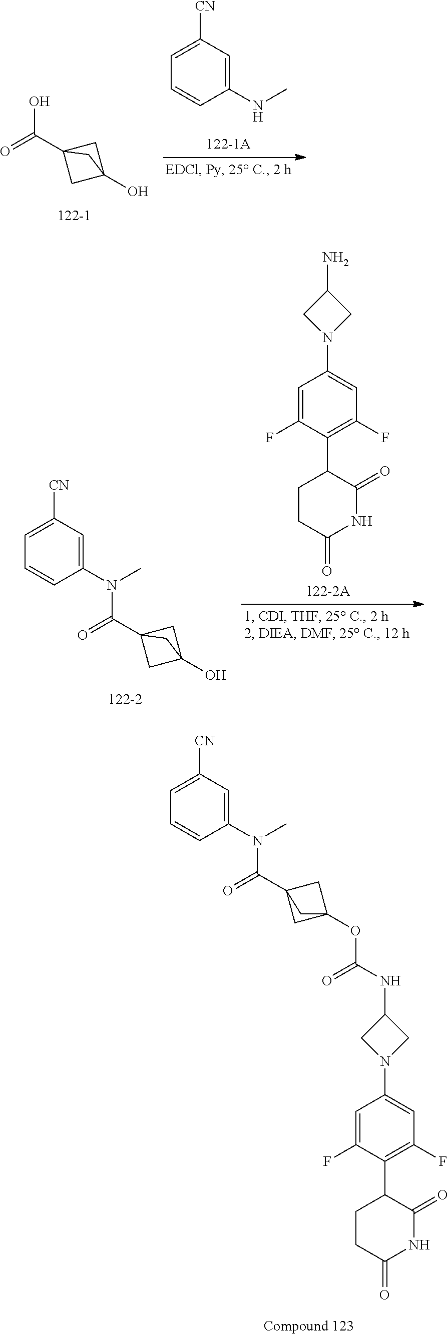

### Step 1. Procedure for Preparation of Compound 2—N-(3-cyanophenyl)-3-hydroxy-N-methylbicyclo[1.1.1]pentane-1-carboxamide

To a solution of 3-(methylamino)benzonitrile (154 mg, 1.17 mmol, 1.00 eq) and 3-hydroxybicyclo[1.1.1]pentane-1-carboxylic acid (150 mg, 1.17 mmol, 1.00 eq) in pyridine (2.00 mL) was added 1-(3-dimethylaminopropyl)-3-ethylcarbodiimide hydrochloride (336 mg, 1.76 mmol, 1.50 eq). The reaction was stirred at 25° C. for 2 h. The reaction was filtered to give a filtrate. The filtrate was purified by reverse phase chromatography (C18, 120 g; condition:water/acetonitrile=100:0 to 60:40, 0.1% formic acid) and lyophilized and Prep-HPLC (column: Phenomenex luna C18 150*25 mm*10 um; mobile phase: [water(formic acid)-acetonitrile]; B %: 8%-38%, 10 min) to afford N-(3-cyanophenyl)-3-hydroxy-N-methylbicyclo[1.1.1]pentane-1-carboxamide (65.0 mg, 268 umol, 23% yield) as a white solid.

1H NMR (400 MHz, DMSO-d6) δ=7.91 (s, 2H), 7.70-7.62 (m, 2H), 6.17 (s, 1H), 3.15 (s, 3H), 1.62 (br s, 6H).

### Step 2. Procedure for Preparation of 3-((3-cyanophenyl)(methyl)carbamoyl)bicyclo[1.1.1]pentan-1-yl (1-(4-(2,6-dioxopiperidin-3-yl)-3,5-difluorophenyl)azetidin-3-yl)carbamate

To a solution of N-(3-cyanophenyl)-3-hydroxy-N-methylbicyclo[1.1.1]pentane-1-carboxamide (65.0 mg, 268 umol, 1.00 eq) in tetrahydrofuran (1.00 mL) was added di(1H-imidazol-1-yl)methanone (43.5 mg, 268 umol, 1.00 eq). The reaction was stirred at 25° C. for 1 h. The resulting solution was added to a solution of 3-(4-(3-aminoazetidin-1-yl)-2,6-difluorophenyl)piperidine-2,6-dione (118 mg, 401 umol, 1.50 eq), N,N-diisopropylethylamine (69.1 mg, 535 umol, 93.2 uL, 2.00 eq) in dimethylformamide (2.00 mL). The reaction was stirred at 25° C. for 12 h. The reaction was filtered to give a residue. The residue was purified by Prep-HPLC (column: Phenomenex luna C18 150*25 mm*10 um; mobile phase: [water(formic acid)-acetonitrile]; B %: 34%-64%, 58 min) to afford 3-((3-cyanophenyl)(methyl)carbamoyl)bicyclo[1.1.1]pentan-1-yl (1-(4-(2,6-dioxopiperidin-3-yl)-3,5-difluorophenyl)azetidin-3-yl)carbamate (8.40 mg, 14.7 umol, 5.51% yield, 99% purity) as a white solid.

1H NMR (400 MHz, DMSO-d6) δ=10.85 (s, 1H), 7.95 (s, 1H), 7.87 (br d, J=8.1 Hz, 1H), 7.77-7.63 (m, 2H), 6.13 (d, J=11.1 Hz, 2H), 4.39-4.29 (m, 1H), 4.08-3.97 (m, 3H), 3.57 (br t, J=6.8 Hz, 2H), 3.16 (s, 3H), 2.82-2.73 (m, 1H), 2.48 (br d, J=3.5 Hz, 1H), 2.11-2.07 (m, 1H), 2.06-1.99 (m, 1H), 1.98-1.83 (m, 6H). 19F NMR (376 MHz, DMSO-d6) δ=−114.05 (s, 1F). MS (ESI) m/z 564.3 [M+H]+.

### Example 123. Synthesis of Compound 124

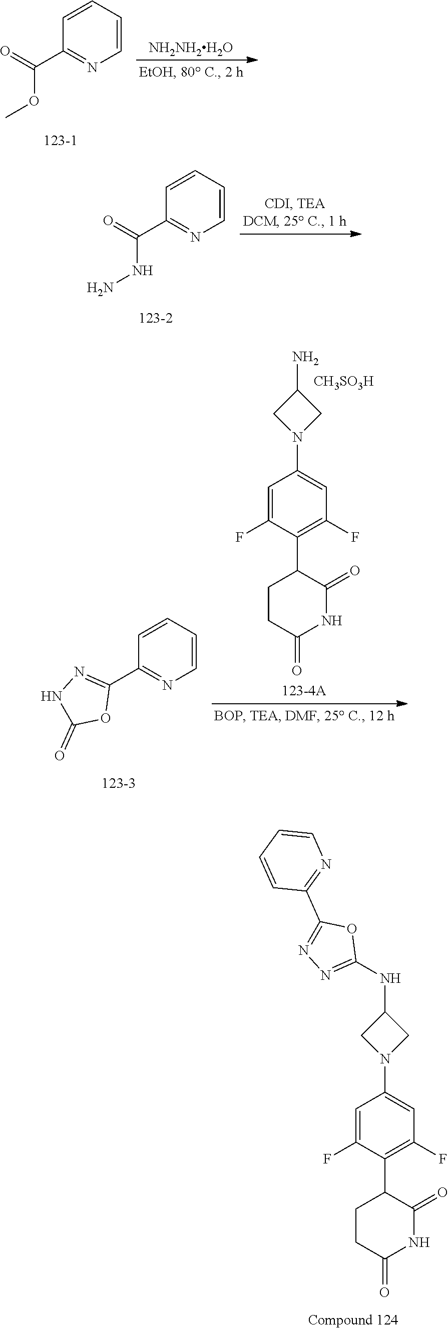

### Step 1. Procedure for Preparation of Compound 2—picolinohydrazide

To a solution of methyl picolinate (5.00 g, 36.4 mmol, 4.39 mL, 1.00 eq) in ethanol (50.0 mL) was added hydrazine hydrate (4.56 g, 72.9 mmol, 4.43 mL, 80.0% purity, 2.00 eq). The mixture was stirred at 80° C. for 2 h. The mixture was concentrated under reduced pressure to afford picolinohydrazide (4.60 g, 33.5 mmol, 92% yield) as a white solid.

1H NMR (400 MHz, DMSO-d6) δ=9.86 (br s, 1H), 8.60 (td, J=1.2, 4.8 Hz, 1H), 8.06-7.90 (m, 2H), 7.63-7.49 (m, 1H), 4.57 (br s, 2H).

### Step 2. Procedure for Preparation of Compound 3—5-(pyridin-2-yl)-1,3,4-oxadiazol-2(3H)-one

To a solution of picolinohydrazide (600 mg, 4.38 mmol, 1.00 eq) in dichloromethane (6.00 mL) was added triethylamine (1.33 g, 13.1 mmol, 1.83 mL, 3.00 eq) and di(1H-imidazol-1-yl)methanone (2.13 g, 13.1 mmol, 3.00 eq). The mixture was stirred at 25° C. for 1 h. The reaction pH was adjusted to 6.0-7.0 with hydrochloric acid (1M). The reaction mixture was diluted with water (30.0 mL) and exacted with dichloromethane (2×30.0 mL). The organic phase was separated, dried over sodium sulfate, filtered and concentrated under reduced pressure to afford 5-(pyridin-2-yl)-1,3,4-oxadiazol-2(3H)-one (160 mg, 980 umol, 22% yield) as a white solid.

1H NMR (400 MHz, DMSO-d6) δ=8.73-8.68 (m, 1H), 8.02-7.96 (m, 1H), 7.93-7.89 (m, 1H), 7.57 (ddd, J=1.2, 4.8, 7.5 Hz, 1H). MS (ESI) m/z 164.1 [M+H]+.

### Step 3. Procedure for Preparation of 3-(2,6-difluoro-4-(3-((5-(pyridin-2-yl)-1,3,4-oxadiazol-2-yl)amino)azetidin-1-yl)phenyl)piperidine-2,6-dione

To a solution of 3-(4-(3-aminoazetidin-1-yl)-2,6-difluorophenyl)piperidine-2,6-dione (23.9 mg, 61.3 umol, 1.00 eq, methanesulfonic acid) in dimethylformamide (2.00 mL) was added triethylamine (6.20 mg, 61.3 umol, 8.53 uL, 1.00 eq), 5-(pyridin-2-yl)-1,3,4-oxadiazol-2(3H)-one (10.0 mg, 61.3 umol, 1.00 eq) and benzotriazol-1-yloxy-tris(dimethylamino)phosphonium;hexafluorophosphate (29.8 mg, 67.4 umol, 1.10 eq). The mixture was stirred at 25° C. for 12 h. The reaction mixture was concentrated under reduced pressure to give a residue. The residue was triturated with dimethylformamide (5.00 mL) and filtered. The filter cake concentrated under reduced pressure to afford 3-(2,6-difluoro-4-(3-((5-(pyridin-2-yl)-1,3,4-oxadiazol-2-yl)amino)azetidin-1-yl)phenyl)piperidine-2,6-dione (18.3 mg, 39.2 umol, 32.0% yield, 94.0% purity) as a yellow solid.

1H NMR (400 MHz, DMSO-d6) δ=10.86 (s, 1H), 8.68 (br d, J=4.5 Hz, 2H), 8.04-7.92 (m, 2H), 7.53 (ddd, J=1.8, 4.9, 6.8 Hz, 1H), 6.22 (d, J=11.0 Hz, 2H), 4.59 (br d, J=1.5 Hz, 1H), 4.23 (t, J=7.6 Hz, 2H), 4.05 (br dd, J=5.1, 12.4 Hz, 1H), 3.83-3.77 (m, 2H), 2.82-2.74 (m, 1H), 2.37-2.29 (m, 1H), 2.12-2.03 (m, 1H), 1.99-1.93 (m, 1H). MS (ESI) m/z 441.2 [M+H]+.

### Example 124. Synthesis of Compound 125

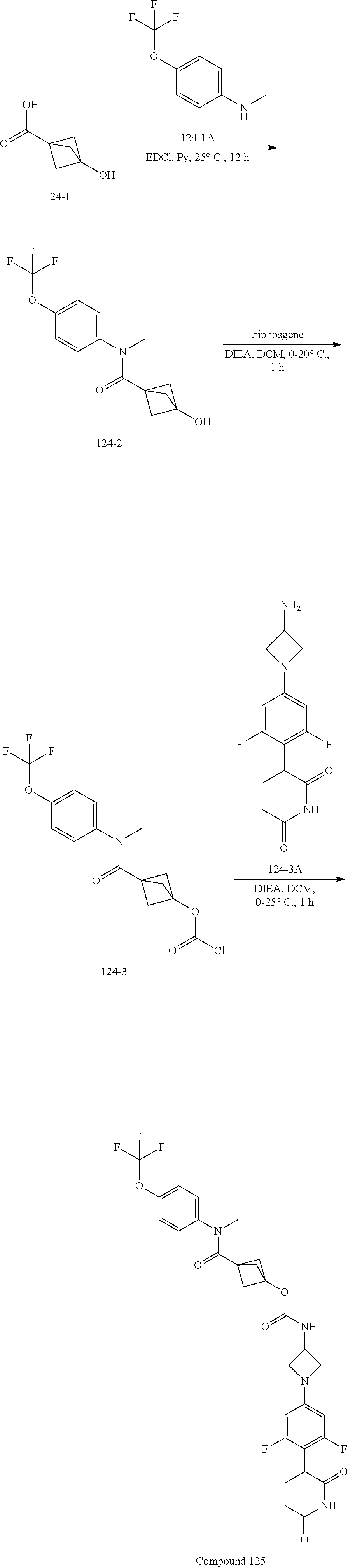 [SMILES: O=C(O)C12CC(O)(C1)C2 ; CNc1ccc(OC(F)(F)F)cc1 ; CN(C(=O)C12CC(O)(C1)C2)c1ccc(OC(F)(F)F)cc1 ; CN(C(=O)C12CC(OC(=O)Cl)(C1)C2)c1ccc(OC(F)(F)F)cc1 ; NC1CN(c2cc(F)c(C3CCC(=O)NC3=O)c(F)c2)C1 ; CN(C(=O)C12CC(OC(=O)NC3CN(c4cc(F)c(C5CCC(=O)NC5=O)c(F)c4)C3)(C1)C2)c1ccc(OC(F)(F)F)cc1]

### Step 1. Procedure for Compound 2—3-hydroxy-N-methyl-N-(4-(trifluoromethoxy)phenyl)bicyclo[1.1.1]pentane-1-carboxamide

To a solution of 3-hydroxybicyclo[1.1.1]pentane-1-carboxylic acid (100 mg, 781 umol, 1.00 eq) in pyridine (1.00 mL) was added 1-(3-dimethylaminopropyl)-3-ethylcarbodiimide hydrochloride (224 mg, 1.17 mmol, 1.50 eq) and N-methyl-4-(trifluoromethoxy)aniline (224 mg, 1.17 mmol, 1.50 eq). The mixture was stirred at 25° C. for 12 h. The reaction mixture was concentrated under reduced pressure to give a residue. The residue was purified by column chromatography (SiO2, petroleum ether/ethyl acetate=1:1) to afford 3-hydroxy-N-methyl-N-(4-(trifluoromethoxy)phenyl)bicyclo[1.1.1]pentane-1-carboxamide (130 mg, 432 umol, 55.29% yield) as yellow oil.

1H NMR (400 MHz, DMSO-d6) δ=7.31-7.27 (m, 2H), 7.26-7.20 (m, 2H), 3.24 (s, 3H), 1.82 (br s, 6H)

### Step 2. Procedure for compound 3—3-(methyl(4-(trifluoromethoxy)phenyl)carbamoyl)bicyclo[1.1.1]pentan-1-yl carbonochloridate

To a solution of 3-hydroxy-N-methyl-N-(4-(trifluoromethoxy)phenyl)bicyclo[1.1.1]pentane-1-carboxamide (60.0 mg, 199 umol, 1.00 eq) in dichloromethane (1.00 mL) was added N,N-diisopropylethylamine (103 mg, 797 umol, 139 uL, 4.00 eq) and triphosgene (47.3 mg, 159 umol, 0.80 eq) at 0° C. The mixture was stirred at 20° C. for 1 h. The reaction mixture was concentrated under reduced pressure to afford 3-(methyl(4-(trifluoromethoxy)phenyl)carbamoyl)bicyclo[1.1.1]pentan-1-yl carbonochloridate (70.0 mg, 192 umol, 96.63% yield) as a yellow solid.

### Step 3. Procedure for 3-(methyl(4-(trifluoromethoxy)phenyl)carbamoyl)bicyclo[1.1.1]pentan-1-yl (1-(4-(2,6-dioxopiperidin-3-yl)-3,5-difluorophenyl)azetidin-3-yl)carbamate

To a solution of 3-(methyl(4-(trifluoromethoxy)phenyl)carbamoyl)bicyclo[1.1.1]pentan-1-yl carbonochloridate (70.0 mg, 232 umol, 1.00 eq) in dichloromethane (1.00 mL) was added N,N-diisopropylethylamine (60.1 mg, 465 umol, 80.9 uL, 2.00 eq) and 3-(4-(3-aminoazetidin-1-yl)-2,6-difluorophenyl)piperidine-2,6-dione (68.6 mg, 232 umol, 1.00 eq) at 0° C. The mixture was stirred at 25° C. for 1 h. The reaction mixture was concentrated under reduced pressure to give a residue. The residue was purified by reverse phase chromatography (C18, 80 g; condition: water/acetonitrile=1:0 to 1:1, 0.1% formid) to afford 3-(methyl(4-(trifluoromethoxy)phenyl)carbamoyl)bicyclo[1.1.1]pentan-1-yl (1-(4-(2,6-dioxopiperidin-3-yl)-3,5-difluorophenyl)azetidin-3-yl)carbamate (36.12 mg, 56.28 umol, 30.22% yield, 97% purity) as a white solid.

1H NMR (400 MHz, DMSO-d6) δ=10.85 (s, 1H), 7.86 (br d, J=7.0 Hz, 1H), 7.47 (d, J=2.5 Hz, 4H), 6.12 (d, J=11.0 Hz, 2H), 4.39-4.25 (m, 1H), 4.06-3.99 (m, 3H), 3.55 (br t, J=6.7 Hz, 2H), 3.14 (br s, 3H), 2.81-2.72 (m, 1H), 2.47 (br d, J=2.6 Hz, 1H), 2.08-2.01 (m, 1H), 1.94 (br d, J=2.6 Hz, 1H), 1.93-1.75 (m, 6H). MS (ESI) m/z 623.3 [M+H]+.

### Example 125. Synthesis of Compound 126

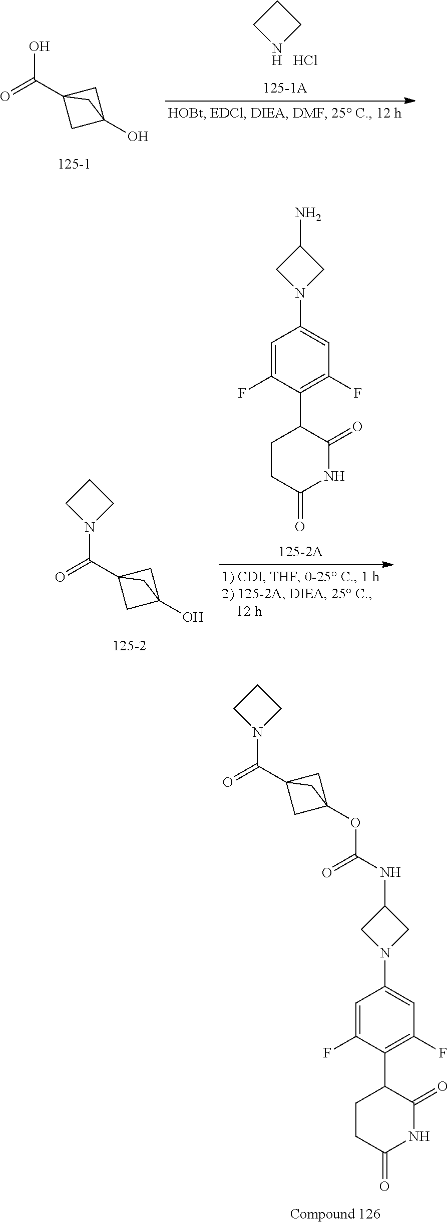

### Step 1. Procedure for Preparation of Compound 2—azetidin-1-yl(3-hydroxybicyclo[1.1.1]pentan-1-yl)methanone

To a solution of 3-hydroxybicyclo[1.1.1]pentane-1-carboxylic acid (50.0 mg, 390 umol, 1.00 eq), azetidineazetidine (183 mg, 1.95 mmol, 5.00 eq, hydrochloride) in dimethyl formamide (1.00 mL) was added 1-hydroxybenzotriazole (36.9 mg, 273 umol, 0.700 eq), 1-(3-dimethylaminopropyl)-3-ethylcarbodiimide hydrochloride (82.3 mg, 429 umol, 1.10 eq) and N,N-diisopropylethylamine (101 mg, 780 umol, 136 uL, 2.00 eq). The mixture was stirred at 25° C. for 12 h. The reaction was filtered. The filtrate was purified by Prep-HPLC (column: Phenomenex luna C18 150*25 mm*10 um; mobile phase: [water (formic acid)-acetonitrile]; B %: 0%-17%, 10 min) and lyophilized to afford azetidin-1-yl(3-hydroxybicyclo[1.1.1]pentan-1-yl)methanone (30.0 mg, 145 umol, 37% yield, 81% purity) as a brown solid.

1H NMR (400 MHz, DMSO-d6) δ=6.34 (s, 1H), 4.15 (t, J=7.7 Hz, 2H), 3.82 (t, J=7.7 Hz, 2H), 2.16 (quin, J=7.7 Hz, 2H), 1.97 (s, 6H).

### Step 2. Procedure for Preparation of 3-(azetidine-1-carbonyl)bicyclo[1.1.1]pentan-1-yl (1-(4-(2,6-dioxopiperidin-3-yl)-3,5-difluorophenyl) azetidin-3-yl)carbamate

To a solution of azetidin-1-yl(3-hydroxybicyclo[1.1.1]pentan-1-yl)methanone (30.0 mg, 179 umol, 1.00 eq) in tetrahydrofuran (1.00 mL) was added di(1H-imidazol-1-yl)methanone (29.1 mg, 179 umol, 1.00 eq) at 0° C. The mixture was stirred at 25° C. for 1 h. The resulting solution was added to a mixture of 3-(4-(3-aminoazetidin-1-yl)-2,6-difluorophenyl)piperidine-2,6-dione (41.6 mg, 141 umol, 0.80 eq, mesylate), N,N-diisopropylethylamine (45.5 mg, 352 umol, 61.3 uL, 2.00 eq) in dimethylformamide (1.00 mL). The mixture was stirred at 25° C. for 12 h. The reaction was filtered. The filtrate was purified by Prep-HPLC (column: Phenomenex luna C18 150*25 mm*10 um; mobile phase: [water(formic acid)-acetonitrile]; B %: 22%-52%, 10 min) and lyophilized to afford 3-(azetidine-1-carbonyl)bicyclo[1.1.1]pentan-1-yl (1-(4-(2,6-dioxopiperidin-3-yl)-3,5-difluorophenyl)azetidin-3-yl)carbamate (17.2 mg, 35.21 umol, 20% yield, 97% purity) as a white solid.

1H NMR (400 MHz, DMSO-d6) δ=10.85 (br s, 1H), 7.95 (d, J=7.4 Hz, 1H), 6.15 (d, J=11.0 Hz, 2H), 4.44-4.33 (m, 1H), 4.19 (t, J=7.6 Hz, 2H), 4.08 (t, J=7.7 Hz, 2H), 4.05-4.00 (m, 1H), 3.85 (t, J=7.8 Hz, 2H), 3.62 (br t, J=6.8 Hz, 2H), 2.83-2.72 (m, 1H), 2.47 (br s, 1H), 2.28 (s, 6H), 2.18 (quin, J=7.7 Hz, 2H), 2.10-2.00 (m, 1H), 1.98-1.90 (m, 1H). MS (ESI) m/z 489.0 [M+H]+.

### Example 126. Synthesis of Compound 127

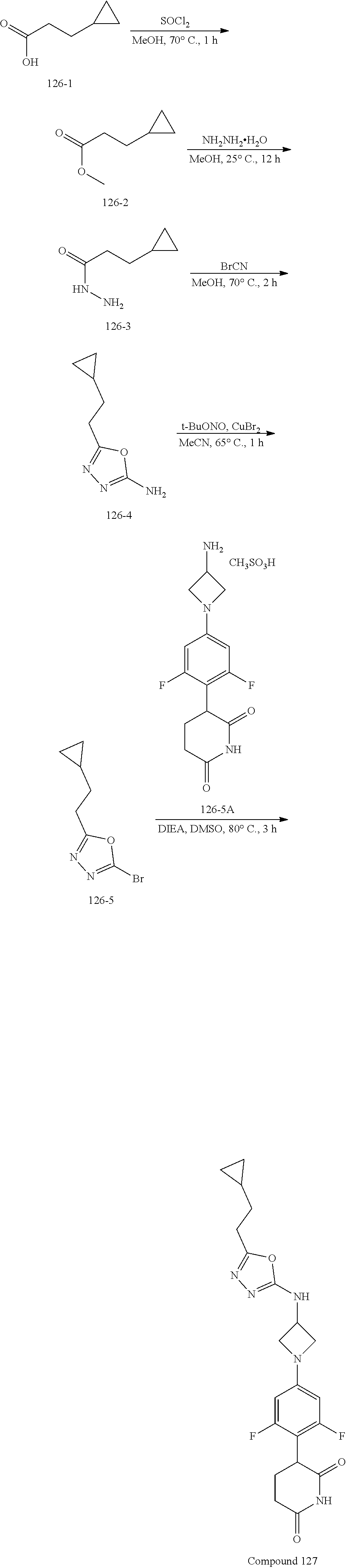

### Step 1. Procedure for Preparation of Compound 2—methyl 3-cyclopropylpropanoate

To a solution of 3-cyclopropylpropanoic acid (1.00 g, 8.76 mmol, 1.00 eq) in methanol (10.0 mL) was added thionyl chloride (3.13 g, 26.3 mmol, 1.91 mL, 3.00 eq) at 0° C. Then the reaction was refluxed at 70° C. for 1 h. The reaction mixture was concentrated under reduced pressure to afford methyl 3-cyclopropylpropanoate (1.12 g, crude) in methanol (10.0 mL) as colorless liquid, which was used into the next step without further purification.

### Step 2. Procedure for Preparation of Compound 3—3-cyclopropylpropanehydrazide

To a solution of methyl 3-cyclopropylpropanoate (1.12 g, crude) in methanol (10.0 mL) was added hydrazinium hydroxide solution (5.15 g, 87.4 mmol, 5.00 mL, 85% purity, 10.0 eq). The reaction was refluxed at 25° C. for 12 h. The reaction mixture was concentrated under reduced pressure to afford 3-cyclopropylpropanehydrazide (1.12 g, crude) as colorless oil.

1H NMR (400 MHz, CDCl3) δ=7.04 (br s, 1H), 4.04-3.56 (m, 2H), 2.27-2.22 (m, 2H), 1.54 (q, J=7.3 Hz, 2H), 0.68 (tquin, J=4.9, 7.5 Hz, 1H), 0.46-0.39 (m, 2H), 0.05 (q, J=4.8 Hz, 2H).

### Step 3. Procedure for Preparation of Compound 4—5-(2-cyclopropylethyl)-1,3,4-oxadiazol-2-amine

To a solution of 3-cyclopropylpropanehydrazide (1.12 g, 8.74 mmol, 1.00 eq, crude) in methanol (10.0 mL) was added cyanic bromide (1.39 g, 13.1 mmol, 964 uL, 1.50 eq). Then the reaction was refluxed at 70° C. for 2 h. The reaction mixture was concentrated under reduced pressure to give a residue. The residue was triturated with ethyl acetate (30.0 mL). The resulting solid was filtered through a funnel and lyophilized to afford 5-(2-cyclopropylethyl)-1,3,4-oxadiazol-2-amine (1.30 g, crude) as colorless oil.

1H NMR (400 MHz, DMSO-d6) δ=10.02-8.57 (m, 2H), 2.78 (t, J=7.4 Hz, 2H), 1.53 (q, J=7.3 Hz, 2H), 0.82-0.73 (m, 1H), 0.43-0.38 (m, 2H), 0.08-0.04 (m, 2H).

### Step 4. Procedure for Preparation of Compound 5—2-bromo-5-(2-cyclopropylethyl)-1,3,4-oxadiazole

To a solution of 5-(2-cyclopropylethyl)-1,3,4-oxadiazol-2-amine (100 mg, 653 umol, 1.00 eq, crude) in acetonitrile (5.00 mL) were added tert-butyl nitrite (135 mg, 1.31 mmol, 155 uL, 2.00 eq), cupric bromide (146 mg, 653 umol, 30.6 uL, 1.00 eq) under nitrogen atmosphere. Then the reaction was stirred at 65° C. for 1 h. The mixture was quenched by saturated sodium bicarbonate (5 mL), then extracted with ethyl acetate (20 mL). The organic layers were washed with brine (10 mL), and dried over anhydrous sodium sulfate, filtered and concentrate to afford 2-bromo-5-(2-cyclopropylethyl)-1,3,4-oxadiazole (70.0 mg, crude) as white solid.

### Step 5. Procedure for Preparation of 3-(4-(3-((5-(2-cyclopropylethyl)-1,3,4-oxadiazol-2-yl)amino)azetidin-1-yl)-2,6-difluorophenyl)piperidine-2,6-dione

To a solution of 2-bromo-5-(2-cyclopropylethyl)-1,3,4-oxadiazole (70.0 mg, 242 umol, crude, 1.00 eq) in dimethylsulfoxide (2.00 mL) were added 3-(4-(3-aminoazetidin-1-yl)-2,6-difluorophenyl)piperidine-2,6-dione (98.5 mg, 242 umol, 1.00 eq, mesylate) and N,N-diisopropylethylamine (46.9 mg, 363 umol, 63.2 uL, 1.50 eq). Then the reaction mixture was stirred at 80° C. for 3 h. The reaction mixture was concentrated under reduced pressure to give a residue. The residue was purified by column chromatography (SiO2, petroleum ether/ethyl acetate=1/1 to 1/3) to afford 3-(4-(3-((5-(2-cyclopropylethyl)-1,3,4-oxadiazol-2-yl)amino)azetidin-1-yl)-2,6-difluorophenyl)piperidine-2,6-dione (17.2 mg, 36.28 umol, 11% yield, 91% purity) as an off-white solid.

1H NMR (400 MHz, DMSO-d6) δ=10.86 (s, 1H), 8.09 (d, J=7.0 Hz, 1H), 6.18 (d, J=11.0 Hz, 2H), 4.47-4.39 (m, 1H), 4.17 (t, J=7.6 Hz, 2H), 4.04 (br dd, J=5.1, 12.5 Hz, 1H), 3.75-3.69 (m, 2H), 2.85-2.76 (m, 1H), 2.73 (t, J=7.4 Hz, 2H), 2.48-2.46 (m, 1H), 2.12-2.04 (m, 1H), 1.97-1.92 (m, 1H), 1.52 (q, J=7.3 Hz, 2H), 0.78-0.68 (m, 1H), 0.42-0.36 (m, 2H), 0.07-0.01 (m, 2H). MS (ESI) m/z 432.2 [M+H]+.

### Example 127. Synthesis of Compound 128

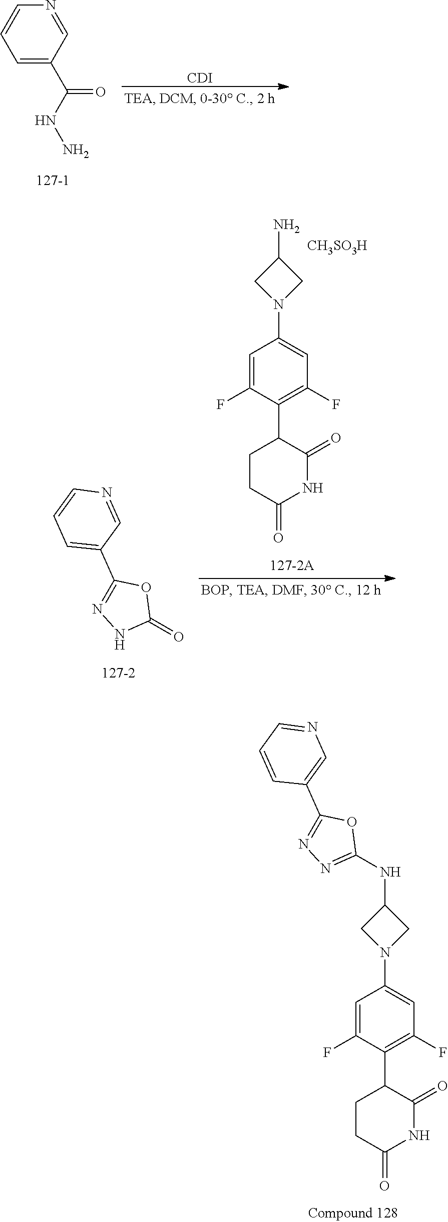

### Step 1. Procedure for Preparation of Compound 2—5-(pyridin-3-yl)-1,3,4-oxadiazol-2(3H)-one

To a solution of nicotinohydrazide (1.00 g, 7.29 mmol, 1.00 eq) in dichloromethane (10.0 mL) were added triethylamine (2.21 g, 21.9 mmol, 3.04 mL, 3.00 eq) and di(1H-imidazol-1-yl)methanone (3.55 g, 21.9 mmol, 3.00 eq) at 0° C. The mixture was stirred at 30° C. for 2 h. The reaction pH was adjust to 6.0˜7.0 with hydrochloric acid (4 M). The mixture was extracted with dichloromethane (3×15.0 mL). The combined organic layers were combined and washed with brine (10.0 mL), dried with anhydrous sodium sulfate, filtered and concentrated under reduced pressure to afford 5-(pyridin-3-yl)-1,3,4-oxadiazol-2(3H)-one (250 mg, 1.53 mmol, 21% yield) as a white solid.

1H NMR (400 MHz, DMSO-d6) δ=8.97 (d, J=1.6 Hz, 1H), 8.77-8.71 (m, 1H), 8.17 (br d, J=8.0 Hz, 1H), 7.63-7.52 (m, 1H), 7.16-7.03 (m, 1H).

### Step 2 Procedure for preparation of 3-(2,6-difluoro-4-(3-((5-(pyridin-3-yl)-1,3,4-oxadiazol-2-yl)amino)azetidin-1-yl)phenyl)piperidine-2,6-dione

To a solution of 3-(4-(3-aminoazetidin-1-yl)-2,6-difluorophenyl)piperidine-2,6-dione (119 mg, 306 umol, 1.00 eq, mesylate) in dimethylformamide (2.00 mL) were added 5-(pyridin-3-yl)-1,3,4-oxadiazol-2(3H)-one (50.0 mg, 306 umol, 1.00 eq), triethylamine (93.0 mg, 919 umol, 128 uL, 3.00 eq) and benzotriazol-1-yloxy-tris(dimethylamino)phosphonium;hexafluorophosphate (136 mg, 305 umol, 1.00 eq). The mixture was stirred at 30° C. for 12 h. The reaction mixture was concentrated under reduce pressure to give a residue. The residue was purified by column chromatography (SiO2, petroleum ether/ethyl acetate=1/3 to 0/1) to afford 3-(2,6-difluoro-4-(3-((5-(pyridin-3-yl)-1,3,4-oxadiazol-2-yl)amino)azetidin-1-yl)phenyl)piperidine-2,6-dione (10.47 mg, 22.82 umol, 7.45% yield, 96% purity) as a white solid.

1H NMR (400 MHz, DMSO-d6) δ=10.87 (s, 1H), 9.01 (d, J=1.5 Hz, 1H), 8.75-8.60 (m, 2H), 8.24-8.12 (m, 1H), 7.64-7.53 (m, 1H), 6.22 (d, J=11.0 Hz, 2H), 4.65-4.53 (m, 1H), 4.24 (t, J=7.7 Hz, 2H), 4.05 (br dd, J=5.2, 12.7 Hz, 1H), 3.87-3.77 (m, 2H), 2.84-2.74 (m, 1H), 2.53 (br s, 1H), 2.13-2.05 (m, 1H), 2.00-1.92 (m, 1H). MS (ESI) m/z. 441.0 [M+H]+.

### Example 128. Synthesis of Compound 129

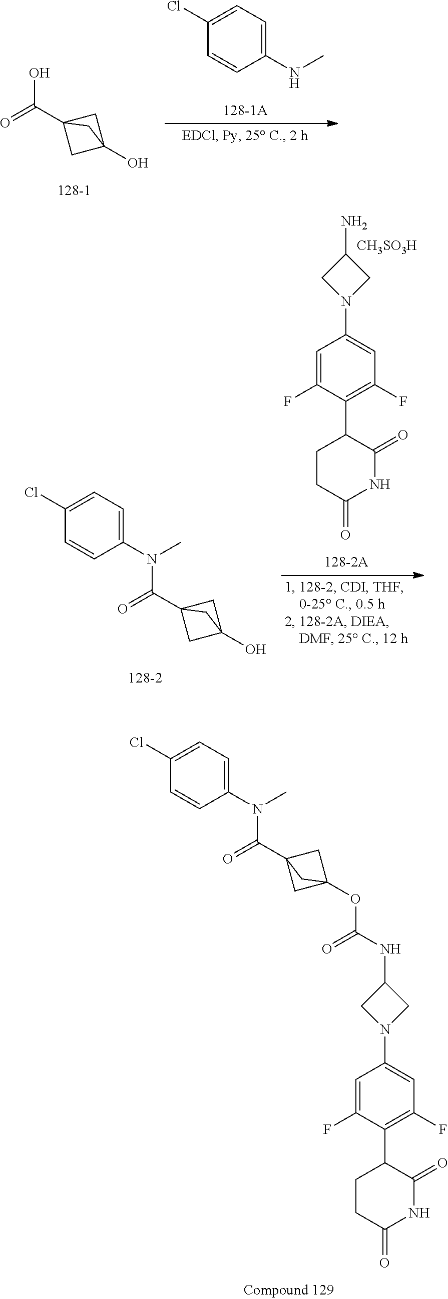

### Step 1. Procedure for Preparation of Compound 2—N-(4-chlorophenyl)-3-hydroxy-N-methylbicyclo[1.1.1]pentane-1-carboxamide

To a solution of 3-hydroxybicyclo[1.1.1]pentane-1-carboxylic acid (100 mg, 780 umol, 1.00 eq), 4-chloro-N-methylaniline (132 mg, 936 umol, 113 uL, 1.20 eq) in pyridine (1.00 mL) was added 1-(3-dimethylaminopropyl)-3-ethylcarbodiimide hydrochloride (224 mg, 1.17 mmol, 1.50 eq). Then the reaction was stirred at 25° C. for 2 h. The reaction mixture was concentrated under reduced pressure to give a residue. The residue was purified by column chromatography (SiO2, petroleum ether/ethyl acetate=5/1 to 1/1) to afford N-(4-chlorophenyl)-3-hydroxy-N-methylbicyclo[1.1.1]pentane-1-carboxamide (150 mg, 578 umol, 74% yield, 97% purity) as an off-white solid. MS (ESI) m/z 251.9 [M+H]+.

### Step 2. Procedure for Preparation of 3-((4-chlorophenyl)(methyl)carbamoyl)bicyclo[1.1.1]pentan-1-yl (1-(4-(2,6-dioxopiperidin-3-yl)-3,5-difluorophenyl)azetidin-3-yl)carbamate

To the solution of N-(4-chlorophenyl)-3-hydroxy-N-methylbicyclo[1.1.1]pentane-1-carboxamide (50.0 mg, 198 umol, 1.00 eq) in tetrahydrofuran (0.500 mL) was added di(1H-imidazol-1-yl)methanone (38.7 mg, 238 umol, 1.2 eq) at 0° C. The reaction mixture was stirred at 25° C. for 0.5 h. Then the mixture were added into a solution of 3-(4-(3-aminoazetidin-1-yl)-2,6-difluorophenyl)piperidine-2,6-dione (77.8 mg, 198 umol, 1.00 eq, mesylate) and N,N-diisopropylethylamine (38.5 mg, 298 umol, 51.9 uL, 1.50 eq) in dimethylformamide (0.500 mL). The reaction solution was stirred at 25° C. for 12 h. The reaction mixture was concentrated under reduced pressure to give a residue. The residue was purified by column chromatography (SiO2, petroleum ether/ethyl acetate=2/1 to 1/1). to afford 3-((4-chlorophenyl)(methyl)carbamoyl)bicyclo[1.1.1]pentan-1-yl (1-(4-(2,6-dioxopiperidin-3-yl)-3,5-difluorophenyl)azetidin-3-yl)carbamate (21.87 mg, 36.64 umol, 18% yield, 96% purity) as a white solid.

1H NMR (400 MHz, DMSO-d6) δ=10.85 (s, 1H), 7.86 (br d, J=7.0 Hz, 1H), 7.53 (d, J=8.5 Hz, 2H), 7.37 (d, J=8.5 Hz, 2H), 6.13 (d, J=10.9 Hz, 2H), 4.37-4.28 (m, 1H), 4.07-4.00 (m, 3H), 3.57 (br t, J=7.0 Hz, 2H), 3.13 (s, 3H), 2.82-2.73 (m, 1H), 2.48-2.46 (m, 1H), 2.07 (br dd, J=3.8, 13.1 Hz, 1H), 1.97-1.85 (m, 7H). MS (ESI) m/z 573.2 [M+H]+.

### Example 129. Synthesis of Compound 130

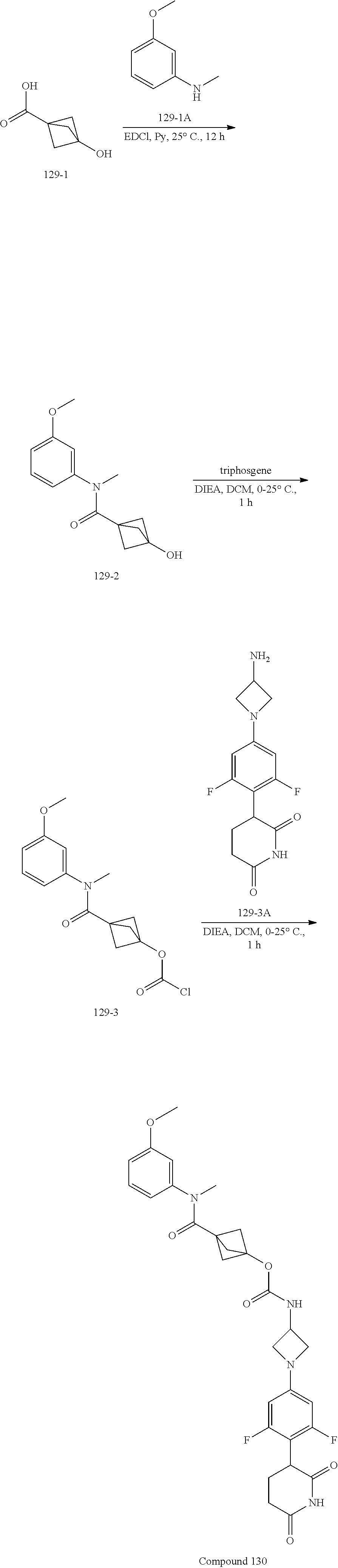

### Step 1. Procedure for Compound 2—3-hydroxy-N-(3-methoxyphenyl)-N-methylbicyclo[1.1.1]pentane-1-carboxamide

To a solution of 3-hydroxybicyclo[1.1.1]pentane-1-carboxylic acid (100 mg, 780 umol, 1.00 eq) in pyridine (1.50 mL) was added 1-(3-dimethylaminopropyl)-3-ethylcarbodiimide hydrochloride (299 mg, 1.56 mmol, 2.00 eq) and 3-methoxy-N-methyl-aniline (128 mg, 937 umol, 122 uL, 1.20 eq) at 25° C. The mixture was stirred at 25° C. for 12 h. The reaction mixture was concentrated under reduced pressure to give a residue. The residue was purified by column chromatography (SiO2, petroleum ether/ethyl acetate=1/1 to 0/1) to afford 3-hydroxy-N-(3-methoxyphenyl)-N-methylbicyclo[1.1.1]pentane-1-carboxamide (130 mg, 526 umol, 67% yield) as a white solid.

1H NMR (400 MHz, DMSO-d6) δ=7.34 (t, J=8.0 Hz, 1H), 6.96 (dd, J=2.1, 8.2 Hz, 1H), 6.90-6.78 (m, 2H), 6.16-5.99 (m, 1H), 3.77 (s, 3H), 3.10 (s, 3H), 1.60 (br s, 6H).

### Step 2. Procedure for Compound 3—3-((3-methoxyphenyl)(methyl)carbamoyl)bicyclo[1.1.1]pentan-1-yl carbonochloridate

To a solution of 3-hydroxy-N-(3-methoxyphenyl)-N-methylbicyclo[1.1.1]pentane-1-carboxamide (60.0 mg, 243 umol, 1.00 eq) in dichloromethane (2.00 mL) was added N,N-diisopropylethylamine (62.7 mg, 485 umol, 84.5 uL, 2.00 eq) and triphosgene (43.2 mg, 146 umol, 0.600 eq) at 0° C. The mixture was stirred at 25° C. for 1 h. The mixture was concentrated under reduced pressure to give 3-((3-methoxyphenyl)(methyl)carbamoyl)bicyclo[1.1.1]pentan-1-yl carbonochloridate (73 mg, crude) as a yellow solid.

### Step 3. Procedure for 3-((3-methoxyphenyl)(methyl)carbamoyl)bicyclo[1.1.1]pentan-1-yl (1-(4-(2,6-dioxopiperidin-3-yl)-3,5-difluorophenyl)azetidin-3-yl)carbamate

To a solution of 3-((3-methoxyphenyl)(methyl)carbamoyl)bicyclo[1.1.1]pentan-1-yl carbonochloridate (58.0 mg, 148 umol, 1.00 eq, methanesulfonic acid) in dichloromethane (2.00 mL) was added N,N-diisopropylethylamine (38.3 mg, 296 umol, 51.6 uL, 2.00 eq) and 3-(4-(3-aminoazetidin-1-yl)-2,6-difluorophenyl)piperidine-2,6-dione (55.0 mg, 178 umol, 1.20 eq) at 0° C. The mixture was stirred at 25° C. for 1 h. The reaction mixture was concentrated under reduced pressure to give a residue. The residue crude product was purified by reverse phase chromatography (C18, 120 g; condition:water/acetonitrile=100:0 to 60:40, 0.1% formic) and lyophilized to afford 3-((3-methoxyphenyl)(methyl)carbamoyl)bicyclo[1.1.1]pentan-1-yl (1-(4-(2,6-dioxopiperidin-3-yl)-3,5-difluorophenyl)azetidin-3-yl)carbamate (34.74 mg, 58.7 umol, 39% yield, 96% purity) as a white solid.

1H NMR (400 MHz, DMSO-d6) δ=10.85 (s, 1H), 7.83 (br d, J=7.0 Hz, 1H), 7.36 (t, J=8.1 Hz, 1H), 7.00-6.95 (m, 1H), 6.92-6.89 (m, 1H), 6.87 (br d, J=7.8 Hz, 1H), 6.12 (d, J=11.1 Hz, 2H), 4.36-4.26 (m, 1H), 4.05-3.99 (m, 3H), 3.78 (s, 3H), 3.55 (br t, J=6.8 Hz, 2H), 3.12 (s, 3H), 2.81-2.72 (m, 1H), 2.38 (br s, 1H), 2.10-2.03 (m, 1H), 1.94 (br d, J=2.5 Hz, 1H), 1.89 (br s, 6H). MS (ESI) m/z. 569.4 [M+H]+.

### Example 130. Synthesis of Compound 131

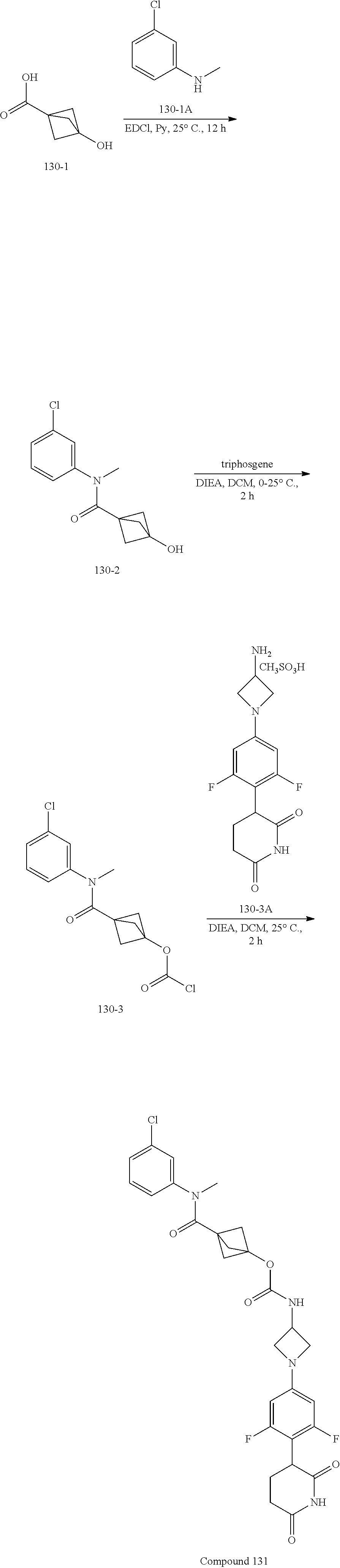

### Step 1. Procedure for Compound 2—N-(3-chlorophenyl)-3-hydroxy-N-methylbicyclo[1.1.1]pentane-1-carboxamide

To a solution of 3-hydroxybicyclo[1.1.1]pentane-1-carboxylic acid (100 mg, 780 umol, 1.00 eq) in pyridine (3.00 mL) were added 3-chloro-N-methylaniline (166 mg, 1.17 mmol, 143 uL, 1.50 eq) and 1-(3-dimethylaminopropyl)-3-ethylcarbodiimide hydrochloride (224 mg, 1.17 mmol, 1.50 eq). The mixture was stirred at 25° C. for 12 h. The mixture was diluted with water (5.00 mL) and extracted with ethyl acetate (3×10.0 mL). The combined organic layer was dried over anhydrous sodium sulfate, filtered and concentrated under reduced pressure to give a residue. The residue was purified by column chromatography (SiO2, petroleum ether/ethyl acetate=10/1 to 1/1) to afford N-(3-chlorophenyl)-3-hydroxy-N-methylbicyclo[1.1.1]pentane-1-carboxamide (70.0 mg, 278 umol, 35% yield) as an off-white solid.

1H NMR (400 MHz, DMSO-d6) δ=7.53-7.42 (m, 3H), 7.33-7.26 (m, 1H), 6.15 (s, 1H), 3.13 (s, 3H), 1.62 (br s, 6H).

### Step 2. Procedure for Preparation of Compound 3—3-((3-chlorophenyl)(methyl)carbamoyl)bicyclo[1.1.1]pentan-1-yl carbonochloridate

To a solution of N-(3-chlorophenyl)-3-hydroxy-N-methylbicyclo[1.1.1]pentane-1-carboxamide (50.0 mg, 198 umol, 1.00 eq) in dichloromethane (2.00 mL) were added N,N-diisopropylethylamine (77.0 mg, 596 umol, 104 uL, 3.00 eq) and triphosgene (47.2 mg, 159 umol, 0.800 eq) at 0° C. The mixture was stirred at 25° C. for 2 h. The mixture was concentrated under reduced pressure to give 3-((3-chlorophenyl)(methyl)carbamoyl)bicyclo[1.1.1]pentan-1-yl carbonochloridate (50.0 mg, 159 umol, 80% yield) as colorless oil.

### Step 3. Procedure for 3-((3-chlorophenyl)(methyl)carbamoyl)bicyclo[1.1.1]pentan-1-yl (1-(4-(2,6-dioxopiperidin-3-yl)-3,5-difluorophenyl)azetidin-3-yl)carbamate

To a solution of 3-(4-(3-aminoazetidin-1-yl)-2,6-difluorophenyl)piperidine-2,6-dione (62.3 mg, 159 umol, 1.00 eq, methanesulfonic acid) in dichloromethane (2.00 mL) were added N,N-diisopropylethylamine (41.1 mg, 318 umol, 55.4 uL, 2.00 eq) and 3-((3-chlorophenyl)(methyl)carbamoyl)bicyclo[1.1.1]pentan-1-yl carbonochloridate (50.0 mg, 159 umol, 1.00 eq). The mixture was stirred at 25° C. for 2 h. The mixture was concentrated under reduced pressure to give a residue. The residue was purified by reverse phase chromatography (C18, 40 g; condition: water/acetonitrile=100:0 to 0:100, 0.1% formic acid) and lyophilized to afford 3-((3-chlorophenyl)(methyl)carbamoyl)bicyclo[1.1.1]pentan-1-yl (1-(4-(2,6-dioxopiperidin-3-yl)-3,5-difluorophenyl)azetidin-3-yl)carbamate (13 mg, 22.23 umol, 13.97% yield, 98% purity) as an white solid.

1H NMR (400 MHz, DMSO-d6) δ=10.85 (br s, 1H), 7.87 (br d, J=7.4 Hz, 1H), 7.55-7.45 (m, 3H), 7.37-7.29 (m, 1H), 6.13 (d, J=11.0 Hz, 2H), 4.38-4.27 (m, 1H), 4.08-3.99 (m, 3H), 3.56 (br t, J=6.6 Hz, 2H), 3.14 (s, 3H), 2.83-2.72 (m, 1H), 2.48 (br d, J=2.6 Hz, 1H), 2.11-2.04 (m, 1H), 1.95 (br s, 1H), 1.95-1.81 (m, 6H). MS (ESI) m/z 573.2 [M+H]+.

### Example 131. Synthesis of Compound 132

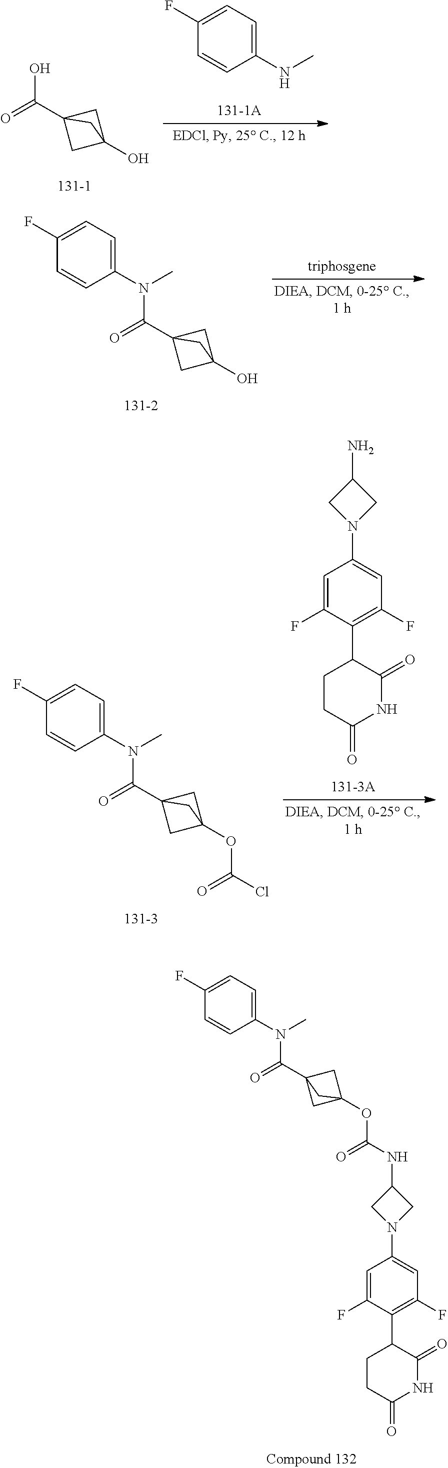

### Step 1. Procedure for Compound 2—N-(4-fluorophenyl)-3-hydroxy-N-methylbicyclo[1.1.1]pentane-1-carboxamide

To a solution of 3-hydroxybicyclo[1.1.1]pentane-1-carboxylic acid (100 mg, 780 umol, 1.00 eq) in pyridine (1.00 mL) was added 1-(3-dimethylaminopropyl)-3-ethylcarbodiimide hydrochloride (224 mg, 1.17 mmol, 1.50 eq) and 4-fluoro-N-methylaniline (147 mg, 1.17 mmol, 141 uL, 1.50 eq). The mixture was stirred at 25° C. for 12 h. The reaction mixture was concentrated under reduced pressure to give a residue. The residue was purified by column chromatography (SiO2, petroleum ether/ethyl acetate=1:2) to afford N-(4-fluorophenyl)-3-hydroxy-N-methylbicyclo[1.1.1]pentane-1-carboxamide (130 mg, 553 umol, 70.80% yield) as off-white solid.

1H NMR (400 MHz, DMSO-d6) δ=7.21-7.06 (m, 4H), 3.22 (s, 3H), 1.81 (s, 6H)

### Step 2. Procedure for Compound 3—3-((4-fluorophenyl)(methyl)carbamoyl)bicyclo[1.1.1]pentan-1-yl carbonochloridate

To a solution of N-(4-fluorophenyl)-3-hydroxy-N-methylbicyclo[1.1.1]pentane-1-carboxamide (65.0 mg, 276 umol, 1.00 eq) in dichloromethane (1.00 mL) was added N,N-diisopropylethylamine (143 mg, 1.11 mmol, 193 uL, 4.00 eq) and triphosgene (65.6 mg, 221 umol, 0.800 eq) at 0° C. The mixture was stirred at 25° C. for 1 h. The reaction mixture was concentrated under reduced pressure to afford 3-((4-fluorophenyl)(methyl)carbamoyl)bicyclo[1.1.1]pentan-1-yl carbonochloridate (81.0 mg, 272 umol, 98.47% yield) as yellow oil.

### Step 3. Procedure for 3-((4-fluorophenyl)(methyl)carbamoyl)bicyclo[1.1.1]pentan-1-yl (1-(4-(2,6-dioxopiperidin-3-yl)-3,5-difluorophenyl)azetidin-3-yl)carbamate

To a solution of 3-((4-fluorophenyl)(methyl)carbamoyl)bicyclo[1.1.1]pentan-1-yl carbonochloridate (80.7 mg, 271 umol, 1.00 eq, methanesulfonic acid) in dichloromethane (1.00 mL) was added N,N-diisopropylethylamine (70.0 mg, 542 umol, 94.4 uL, 2.00 eq) and 3-(4-(3-aminoazetidin-1-yl)-2,6-difluorophenyl)piperidine-2,6-dione (80.0 mg, 271 umol, 1.00 eq) at 0° C. The mixture was stirred at 25° C. for 1 h. The reaction mixture was concentrated under reduced pressure to give a residue. The residue was purified by reverse phase chromatography (C18, 80 g; condition: water/acetonitrile=1:0 to 1:1, 0.1% formid) and lyophilized to afford 3-((4-fluorophenyl)(methyl)carbamoyl)bicyclo[1.1.1]pentan-1-yl (1-(4-(2,6-dioxopiperidin-3-yl)-3,5-difluorophenyl)azetidin-3-yl)carbamate (30.67 mg, 54.6 umol, 20.14% yield, 99% purity) as a white solid.

1H NMR (400 MHz, DMSO-d6) δ=10.84 (s, 1H), 7.85 (br d, J=7.1 Hz, 1H), 7.42-7.35 (m, 2H), 7.33-7.26 (m, 2H), 6.12 (br d, J=11.1 Hz, 2H), 4.39-4.25 (m, 1H), 4.06-3.99 (m, 3H), 3.55 (br t, J=6.6 Hz, 2H), 3.11 (s, 3H), 2.81-2.73 (m, 1H), 2.47 (br s, 1H), 2.06 (br dd, J=3.4, 13.5 Hz, 1H), 1.96-1.91 (m, 1H), 1.91-1.80 (m, 6H). MS (ESI) m/z 557.3 [M+H]+.

### Example 132. Synthesis of Compound 133

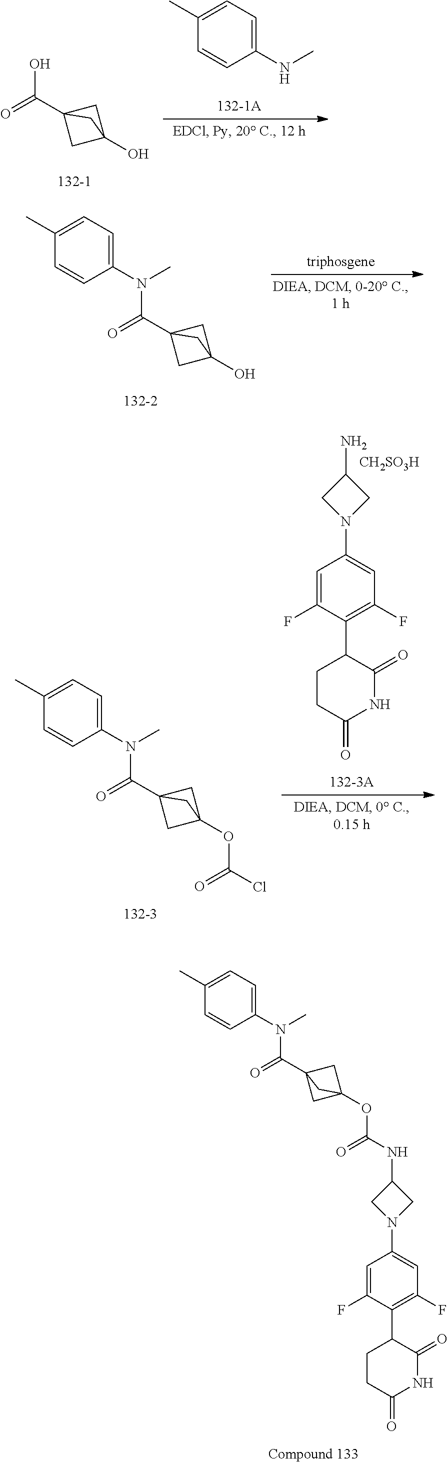

### Step 1. Procedure for Preparation of Compound 2—3-hydroxy-N-methyl-N-(p-tolyl)bicyclo[1.1.1]pentane-1-carboxamide

To a solution of 3-hydroxybicyclo[1.1.1]pentane-1-carboxylic acid (100 mg, 780 umol, 1.00 eq) in pyridine (2.00 mL) was added 1-(3-dimethylaminopropyl)-3-ethylcarbodiimide hydrochloride (224 mg, 1.17 mmol, 1.50 eq) and N, 4-dimethylaniline (142 mg, 1.17 mmol, 148 uL, 1.50 eq). The reaction mixture was stirred at 20° C. for 12 h. The mixture was diluted with saturated copper sulfate solution (10.0 mL) and extracted with ethyl acetate (3×10.0 mL). The combined organic layers was washed with brine (2×10.0 mL), dried over sodium sulfate, filtered and concentrated under reduced pressure to afford 3-hydroxy-N-methyl-N-(p-tolyl)bicyclo[1.1.1]pentane-1-carboxamide (150 mg, crude) as a brown solid. MS (ESI) m/z 232.0 [M+H]+.

### Step 2. Procedure for Preparation of Compound 4—3-(methyl(p-tolyl)carbamoyl)bicycle[1.1.1]pentan-1-yl carbonochloridate

To a solution of 3-hydroxy-N-methyl-N-(p-tolyl)bicyclo[1.1.1]pentane-1-carboxamide (60.0 mg, crude) in dichloromethane (3.00 mL) were added bis(trichloromethyl) carbonate (77.0 mg, 259 umol, 1.00 eq) and N,N-diisopropylethylamine (134 mg, 1.04 mmol, 181 uL, 4.00 eq) at 0° C. Then the reaction was stirred at 20° C. for 1 h. The reaction mixture was concentrated under reduced pressure to afford 3-(methyl(p-tolyl)carbamoyl)bicyclo[1.1.1]pentan-1-yl carbonochloridate (75.0 mg, crude) as a brown solid.

### Step 3 Procedure for preparation of 3-(methyl(p-tolyl)carbamoyl)bicycle [1.1.1]pentan-1-yl (1-(4-(2,6-dioxopiperidin-3-yl)-3,5-difluorophenyl)azetidin-3-yl)carbamate

To a solution of 3-(4-(3-aminoazetidin-1-yl)-2,6-difluorophenyl)piperidine-2,6-dione (100 mg, 256 umol, 1.00 eq, mesylate) in dimethylformamide (2.00 mL) were added N,N-diisopropylethylamine (66.0 mg, 511. umol, 89.0 uL, 2.00 eq) and 3-(methyl(p-tolyl)carbamoyl)bicyclo[1.1.1]pentan-1-yl carbonochloridate (75.0 mg, crude). Then the reaction was stirred at 0° C. for 0.15 h. The reaction mixture was diluted with dimethylformamide (1.00 mL) and then filtered. The filtrate was purified by reverse phase chromatography (C18, 40 g; condition: water/acetonitrile=100:0 to 0:100, 0.1% formic) and lyophilized to afford 3-(methyl (p-tolyl)carbamoyl)bicyclo[1.1.1]pentan-1-yl (1-(4-(2,6-dioxopiperidin-3-yl)-3,5-difluorophenyl) azetidin-3-yl)carbamate (26.11 mg, 46.78 umol, 18.31% yield, 99% purity) as a white solid.

1H NMR (400 MHz, DMSO-d6) δ=10.84 (s, 1H), 7.82 (br d, J=7.0 Hz, 1H), 7.26 (br d, J=8.0 Hz, 2H), 7.20-7.14 (m, 2H), 6.12 (d, J=11.1 Hz, 2H), 4.38-4.24 (m, 1H), 4.05 (br d, J=1.4 Hz, 1H), 4.03-3.97 (m, 2H), 3.55 (br t, J=6.7 Hz, 2H), 3.10 (s, 3H), 2.82-2.72 (m, 1H), 2.47 (br s, 1H), 2.35 (s, 3H), 2.08-2.03 (m, 1H), 1.97-1.91 (m, 1H), 1.85 (br s, 6H). MS (ESI) m/z 553.1 [M+H]+.

### Example 133. Synthesis of Compound 134

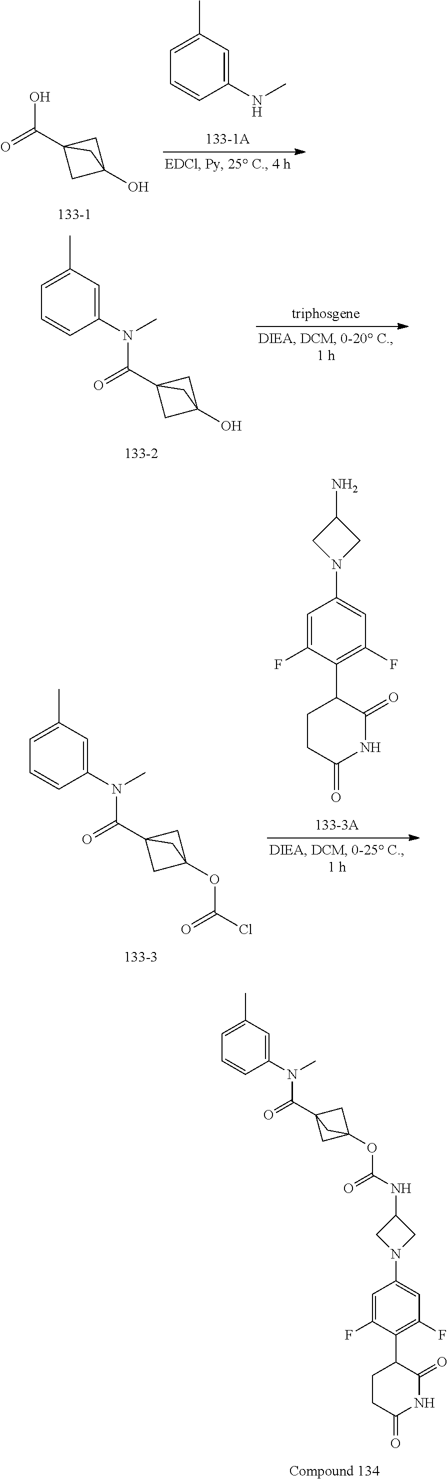

### Step 1. Procedure for Compound 2—3-hydroxy-N-methyl-N-(m-tolyl)bicyclo[1.1.1]pentane-1-carboxamide

To a solution of 3-hydroxybicyclo[1.1.1]pentane-1-carboxylic acid (100 mg, 780 umol, 1.00 eq) in pyridine (1.00 mL) were added 1-(3-dimethylaminopropyl)-3-ethylcarbodiimide hydrochloride (224 mg, 1.17 mmol, 1.50 eq) and N, 3-dimethylaniline (113 mg, 937 umol, 117 uL, 1.20 eq). The mixture was stirred at 25° C. for 4 h. The mixture was concentrated under reduced pressure to give a residue. The residue was purified by column chromatography (SiO2, petroleum ether/ethyl acetate=1/0 to 1/1) to afford 3-hydroxy-N-methyl-N-(m-tolyl)bicyclo[1.1.1]pentane-1-carboxamide (150 mg, 642 umol, 82% yield, 99% purity) as a white solid.

1H NMR (400 MHz, DMSO-d6) δ=7.35-7.29 (m, 1H), 7.20 (br d, J=7.6 Hz, 1H), 7.10-7.03 (m, 2H), 6.07 (s, 1H), 3.09 (s, 3H), 2.33 (s, 3H), 1.56 (br s, 6H).

### Step 2. Procedure for Compound 3—3-(methyl(m-tolyl)carbamoyl)bicyclo[1.1.1]pentan-1-yl carbonochloridate

To a solution of 3-hydroxy-N-methyl-N-(m-tolyl)bicyclo[1.1.1]pentane-1-carboxamide (50.0 mg, 216 umol, 1.00 eq) in dichloromethane (2.00 mL) was added N,N-diisopropylethylamine (55.9 mg, 432 umol, 75.3 uL, 2.00 eq) and triphosgene (38.5 mg, 130 umol, 0.600 eq) at 0° C. The mixture was stirred at 20° C. for 1 h. The mixture was concentrated under reduced pressure to afford 3-(methyl(m-tolyl)carbamoyl)bicyclo[1.1.1]pentan-1-yl carbonochloridate (63.0 mg, crude) as a white solid.

### Step 3. Procedure for 3-(methyl(m-tolyl)carbamoyl)bicyclo[1.1.1]pentan-1-yl (1-(4-(2,6-dioxopiperidin-3-yl)-3,5-difluorophenyl)azetidin-3-yl)carbamate

To a solution of 3-(4-(3-aminoazetidin-1-yl)-2,6-difluorophenyl)piperidine-2,6-dione (56 mg, 143.08 umol, 1 eq, mesylate) in dichloromethane (1.00 mL) was added N,N-diisopropylethylamine (18.5 mg, 143 umol, 24.9 uL, 1.00 eq) and 3-(methyl(m-tolyl)carbamoyl)bicyclo[1.1.1]pentan-1-yl carbonochloridate (47.5 mg, 162 umol, 1.13 eq) at 0° C. The mixture was stirred at 25° C. for 1 h. The mixture was concentrated under reduced pressure to give a residue. The residue was purified by prep-HPLC (C18, 120 g; condition:water/acetonitrile=100:0 to 60:40, 0.1% formic) and lyophilized to afford 3-(methyl(m-tolyl)carbamoyl)bicyclo[1.1.1]pentan-1-yl (1-(4-(2,6-dioxopiperidin-3-yl)-3,5-difluorophenyl)azetidin-3-yl)carbamate (32.52 mg, 58.3 umol, 40% yield, 99% purity) as an white solid.

1H NMR (400 MHz, DMSO-d6) δ=10.84 (s, 1H), 7.82 (br d, J=7.6 Hz, 1H), 7.38-7.31 (m, 1H), 7.22 (br d, J=7.4 Hz, 1H), 7.14-7.06 (m, 2H), 6.12 (br d, J=11.1 Hz, 2H), 4.37-4.23 (m, 1H), 4.06-3.99 (m, 3H), 3.55 (br t, J=6.4 Hz, 2H), 3.12 (s, 3H), 2.81-2.72 (m, 1H), 2.47 (br s, 1H), 2.34 (s, 3H), 2.06 (br dd, J=2.9, 13.4 Hz, 1H), 1.96-1.92 (m, 1H), 1.91-1.78 (m, 6H). MS (ESI) m/z. 553.3 [M+H]+.

### Example 134. Synthesis of Compound 135

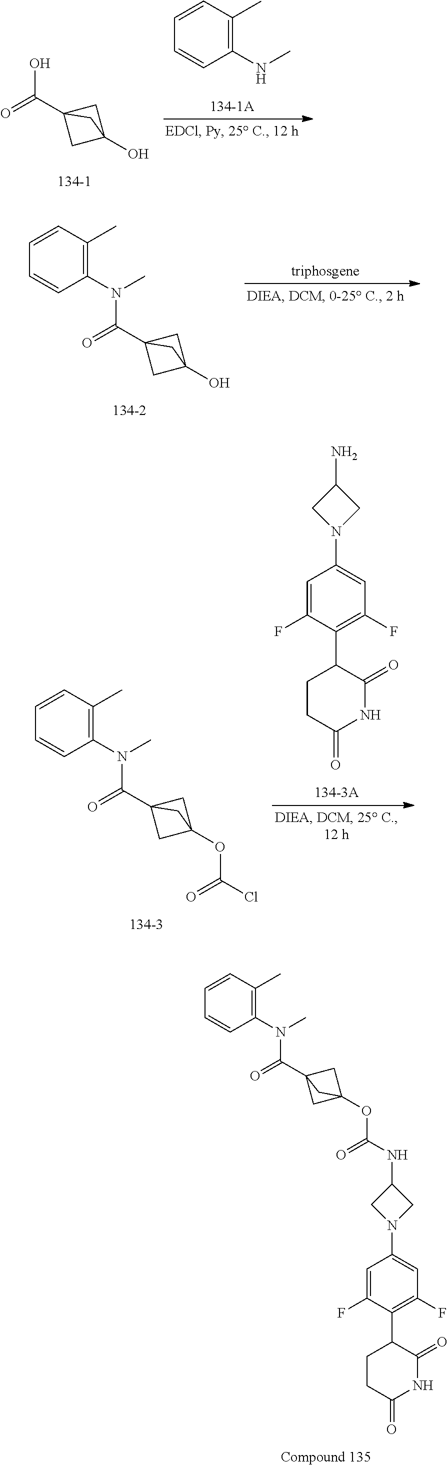

### Step 1. Procedure for Preparation of Compound 2—3-hydroxy-N-methyl-N-(o-tolyl)bicyclo[1.1.1]pentane-1-carboxamide

To a solution of 3-hydroxybicyclo[1.1.1]pentane-1-carboxylic acid (100 mg, 780 umol, 1.00 eq) in pyridine (2.00 mL) was added 1-(3-dimethylaminopropyl)-3-ethylcarbodiimide hydrochloride (224 mg, 1.17 mmol, 1.50 eq) and N, 2-dimethylaniline (141 mg, 1.17 mmol, 144 uL, 1.50 eq). The mixture was stirred at 25° C. for 12 h. The reaction mixture was diluted with water (10.0 mL) and exacted with ethyl acetate (2×10.0 mL). The organic phase was washed with brine (5.00 mL), dried over sodium sulfate, filtered and concentrated under reduced pressure to give a residue. The residue was purified by column chromatography (SiO2, petroleum ether/ethyl acetate=10/1 to ethyl acetate/petroleum ether=3/1) to afford 3-hydroxy-N-methyl-N-(o-tolyl)bicyclo[1.1.1]pentane-1-carboxamide (100 mg, 432 umol, 55% yield) as an white solid.

1H NMR (400 MHz, DMSO-d6) δ=7.37-7.25 (m, 3H), 7.22-7.15 (m, 1H), 6.07 (s, 1H), 3.04 (s, 3H), 2.16 (s, 3H), 1.60-1.55 (m, 3H), 1.50-1.46 (m, 3H).

### Step 2. Procedure for Preparation of Compound 3—3-(methyl(o-tolyl)carbamoyl)bicyclo[1.1.1]pentan-1-yl carbonochloridate

To a solution of 3-hydroxy-N-methyl-N-(o-tolyl)bicyclo[1.1.1]pentane-1-carboxamide (50.0 mg, 216 umol, 1.00 eq) in dichloromethane (2.00 mL) was added N,N-diisopropylethylamine (83.8 mg, 648 umol, 112 uL, 3.00 eq) and triphosgene (76.9 mg, 259 umol, 1.20 eq) at 0° C. The mixture was stirred at 25° C. for 2 h. The mixture was concentrated under reduced pressure to give 3-(methyl(o-tolyl)carbamoyl)bicyclo[1.1.1]pentan-1-yl carbonochloridate (60.0 mg, 204 umol, 94% yield) as yellow oil.

### Step 3. Procedure for Preparation of 3-(methyl(o-tolyl)carbamoyl)bicyclo[1.1.1]pentan-1-yl (1-(4-(2,6-dioxopiperidin-3-yl)-3,5-difluorophenyl)azetidin-3-yl)carbamate

To a solution of 3-(4-(3-aminoazetidin-1-yl)-2,6-difluorophenyl)piperidine-2,6-dione (73.2 mg, 187 umol, 1.00 eq, methanesulfonic acid) in dichloromethane (1.00 mL) was added N,N-diisopropylethylamine (48.4 mg, 374 umol, 65.2 uL, 2.00 eq) and 3-(methyl(o-tolyl)carbamoyl)bicyclo[1.1.1]pentan-1-yl carbonochloridate (55.0 mg, 187 umol, 1.00 eq). The mixture was stirred at 25° C. for 12 h. The reaction mixture was concentrated under reduced pressure to give a residue. The residue was purified by reverse phase chromatography (C18, 40 g, condition: water/acetonitrile=100:0 to 0:100, 0.1% formic acid) and lyophilized to afford 3-(methyl(o-tolyl)carbamoyl)bicyclo[1.1.1]pentan-1-yl (1-(4-(2,6-dioxopiperidin-3-yl)-3,5-difluorophenyl)azetidin-3-yl)carbamate (36.8 mg, 66.0 umol, 35.2% yield, 99.0% purity) as an white solid.

1H NMR (400 MHz, DMSO-d6) δ=10.85 (s, 1H), 7.83 (br d, J=7.3 Hz, 1H), 7.40-7.32 (m, 2H), 7.32-7.26 (m, 1H), 7.25-7.19 (m, 1H), 6.12 (br d, J=11.1 Hz, 2H), 4.38-4.23 (m, 1H), 4.03 (br t, J=7.9 Hz, 3H), 3.55 (br t, J=6.8 Hz, 2H), 3.06 (s, 3H), 2.83-2.72 (m, 1H), 2.63-2.54 (m, 1H), 2.17 (s, 3H), 2.11-2.03 (m, 1H), 1.97-1.90 (m, 1H), 1.90-1.84 (m, 3H), 1.81-1.74 (m, 3H). MS (ESI) m/z 553.2 [M+H]+.

### Example 135. Synthesis of Compound 136

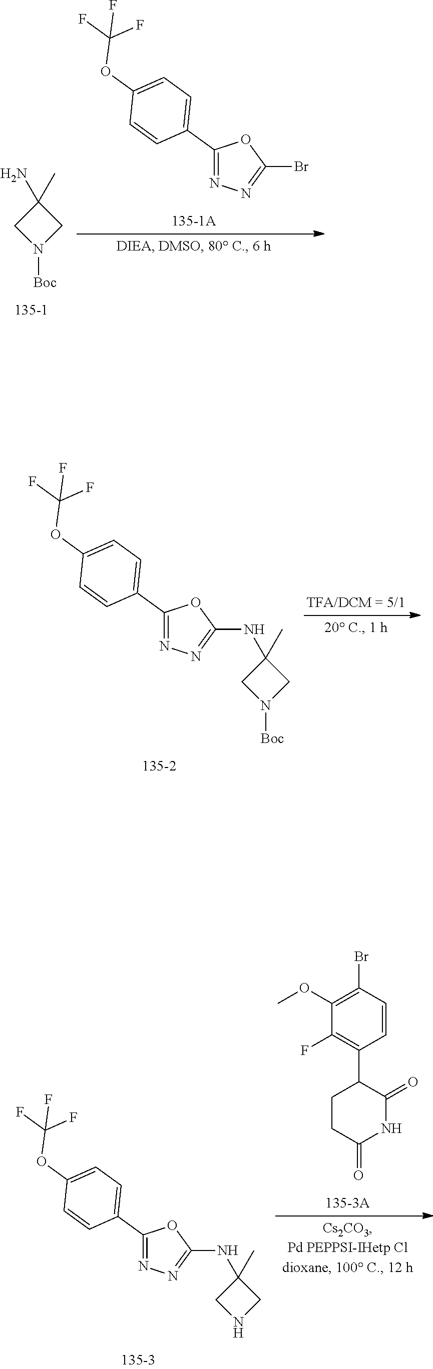 [SMILES: CC1(N)CN(C(=O)OC(C)(C)C)C1 ; FC(F)(F)Oc1ccc(-c2nnc(Br)o2)cc1 ; CC1(Nc2nnc(-c3ccc(OC(F)(F)F)cc3)o2)CN(C(=O)OC(C)(C)C)C1 ; CC1(Nc2nnc(-c3ccc(OC(F)(F)F)cc3)o2)CNC1 ; COc1c(Br)ccc(C2CCC(=O)NC2=O)c1F]

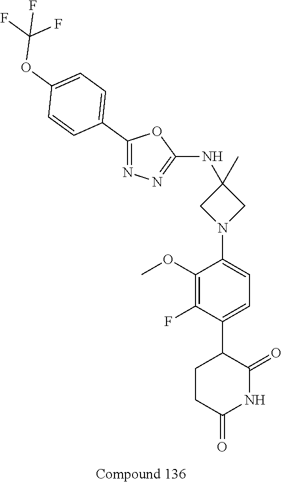 [SMILES: COc1c(N2CC(C)(Nc3nnc(-c4ccc(OC(F)(F)F)cc4)o3)C2)ccc(C2CCC(=O)NC2=O)c1F]

### Step 1. Procedure for Preparation of Compound 2—tert-butyl 3-methyl-3-((5-(4-(trifluoromethoxy)phenyl)-1,3,4-oxadiazol-2-yl)amino)azetidine-1-carboxylate

To a solution of tert-butyl 3-amino-3-methylazetidine-1-carboxylate (100 mg, 536 umol, 1.00 eq) in dimethylsulfoxide (2.00 mL) was added N,N-diisopropylethylamine (138 mg, 1.07 mmol, 187 uL, 2.00 eq) and 2-bromo-5-(4-(trifluoromethoxy)phenyl)-1,3,4-oxadiazole (174 mg, 563 umol, 1.05 eq). The mixture was stirred at 80° C. for 6 h. The reaction mixture was concentrated under reduced pressure to give a residue. The residue was purified by column chromatography (SiO2, petroleum ether/ethyl acetate=100/1 to 3/1) to afford tert-butyl 3-methyl-3-((5-(4-(trifluoromethoxy)phenyl)-1,3,4-oxadiazol-2-yl)amino)azetidine-1-carboxylate (200 mg, 482 umol, 89% yield) as a yellow solid.

1H NMR (400 MHz, CDCl3) δ=7.91 (d, J=8.9 Hz, 2H), 7.29 (d, J=8.1 Hz, 2H), 4.18 (d, J=9.0 Hz, 2H), 3.90 (d, J=8.9 Hz, 2H), 1.72 (br s, 3H), 1.43 (s, 9H).

### Step 2. Procedure for Preparation of Compound 3—N-(3-methylazetidin-3-yl)-5-(4-(trifluoromethoxy)phenyl)-1,3,4-oxadiazol-2-amine

To a solution of tert-butyl 3-methyl-3-((5-(4-(trifluoromethoxy)phenyl)-1,3,4-oxadiazol-2-yl)amino)azetidine-1-carboxylate (100 mg, 241 umol, 1.00 eq) in dichloromethane (2.50 mL) was added trifluoroacetic acid (770 mg, 6.75 mmol, 0.500 mL, 27.9 eq). The mixture was stirred at 20° C. for 1 h. The reaction mixture was concentrated under reduced pressure to give N-(3-methylazetidin-3-yl)-5-(4-(trifluoromethoxy)phenyl)-1,3,4-oxadiazol-2-amine (70.0 mg, 222 umol, 92% yield) as yellow oil. MS (ESI) m/z 315.0 [M+H]+

### Step 3. Procedure for Preparation of 3-(2-fluoro-3-methoxy-4-(3-methyl-3-((5-(4-(trifluoromethoxy)phenyl)-1,3,4-oxadiazol-2-yl)amino)azetidin-1-yl)phenyl)piperidine-2,6-dione

A mixture of N-(3-methylazetidin-3-yl)-5-(4-(trifluoromethoxy)phenyl)-1,3,4-oxadiazol-2-amine (70.0 mg, 222 umol, 1.00 eq), 3-(4-bromo-2-fluoro-3-methoxyphenyl)piperidine-2,6-dione (70.4 mg, 222 umol, 1.00 eq), cesium carbonate (217 mg, 668 umol, 3.00 eq), 1,3-bis[2,6-bis(1-propylbutyl)phenyl]-4,5-dichloro-2H-imidazol-1-ium-2-ide;3-chloropyridine;dichloropalladium (21.6 mg, 22.2 umol, 0.100 eq) in dioxane (2.00 mL) was degassed and purged with nitrogen atmosphere for 3 times. The mixture was stirred at 100° C. for 12 h under nitrogen atmosphere. The reaction mixture was concentrated under reduced pressure to give a residue. The residue was purified by prep-HPLC (column: Phenomenex luna C18 150*25 mm*10 um; mobile phase: [water (formic acid)-acetonitrile]; B %: 38%-68%, 10 min) and lyophilized to afford 3-(2-fluoro-3-methoxy-4-(3-methyl-3-((5-(4-(trifluoromethoxy)phenyl)-1,3,4-oxadiazol-2-yl)amino)azetidin-1-yl)phenyl)piperidine-2,6-dione (8.70 mg, 15.6 umol, 3.50% yield, 98.5% purity) as an white solid.

1H NMR (400 MHz, DMSO-d6) δ=10.92-10.69 (m, 1H), 8.48 (s, 1H), 7.91 (d, J=8.9 Hz, 2H), 7.54 (br d, J=8.1 Hz, 2H), 6.82 (t, J=8.1 Hz, 1H), 6.27 (d, J=8.8 Hz, 1H), 4.09 (d, J=7.8 Hz, 2H), 3.93-3.85 (m, 3H), 3.71 (s, 3H), 2.77-2.70 (m, 1H), 2.67 (br d, J=4.6 Hz, 1H), 2.18 (br s, 1H), 2.01-1.93 (m, 1H), 1.69 (s, 3H). MS (ESI) m/z 550.3 [M+H]⁺.

### Example 136. Synthesis of Compound 137

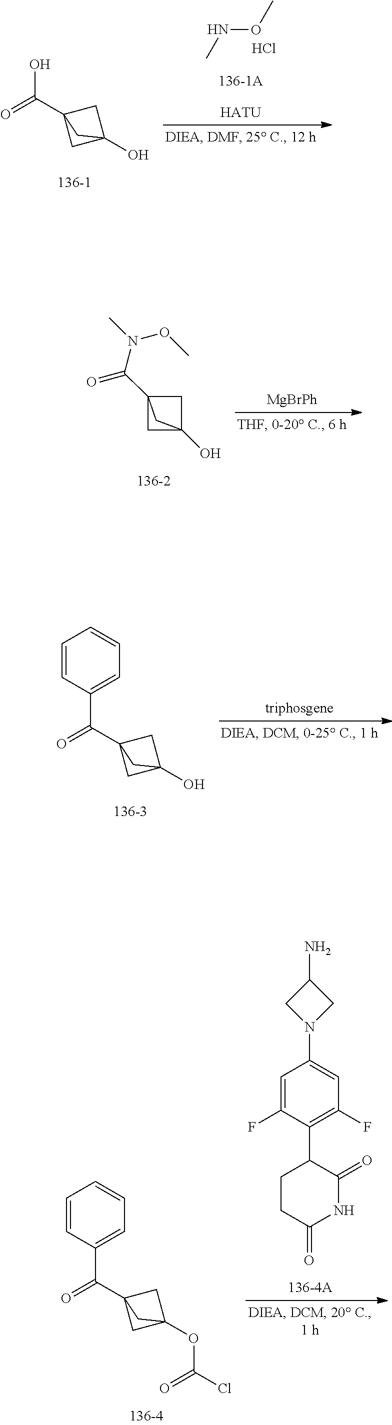

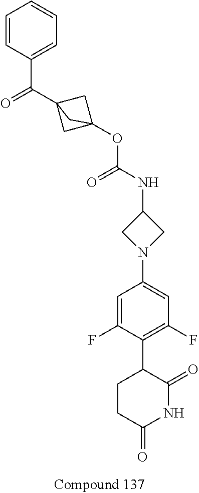 [SMILES: O=C1CCC(c2c(F)cc(N3CC(NC(=O)OC45CC(C(=O)c6ccccc6)(C4)C5)C3)cc2F)C(=O)N1]

### Step 1. Procedure for Preparation of Compound 2—3-hydroxy-N-methoxy-N-methylbicyclo[1.1.1]pentane-1-carboxamide

To a solution of 3-hydroxybicyclo[1.1.1]pentane-1-carboxylic acid (100 mg, 780 umol, 1.00 eq) in dimethyl formamide (1.00 mL) was added o-(7-azabenzotriazol-1-yl)-N,N,N′,N′-tetramethyluronium hexafluorophosphate (356.12 mg, 936.58 umol, 1.2 eq), N,N-diisopropylethylamine (403 mg, 3.12 mmol, 544 uL, 4.00 eq) at 25° C. to afford mixture A. To a solution of N,O-dimethylhydroxylamine (91.4 mg, 937 umol, 1.20 eq, hydrochloride) in dimethyl formamide (1.00 mL) was added N,N-diisopropylethylamine (202 mg, 1.56 mmol, 272 uL, 2.00 eq) to give mixture B. The mixture B was added into the mixture A at 25° C. The mixture was stirred at 25° C. for 12 h. The reaction mixture was concentrated under reduced pressure to give a residue. The residue was purified by column chromatography (SiO2, petroleum ether/ethyl acetate=3/1 to 1/1) to afford 3-hydroxy-N-methoxy-N-methylbicyclo[1.1.1]pentane-1-carboxamide (50.0 mg, 292 umol, 37.42% yield) as colorless oil.

1H NMR (400 MHz, CDCl3-d) δ=3.67 (s, 3H), 3.20 (s, 3H), 2.30-2.23 (m, 6H)

### Step 2. Procedure for Preparation of Compound 3—(3-hydroxybicyclo[1.1.1]pentan-1-yl)(phenyl)methanone

A solution of 3-hydroxy-N-methoxy-N-methylbicyclo[1.1.1]pentane-1-carboxamide (210 mg, 1.23 mmol, 1.00 eq) in tetrahydrofuran (10.0 mL) was degassed and purged with nitrogen atmosphere. The resulting clear solution was cooled to 0° C. And then a solution of phenylmagnesium bromide (3 M, 1.23 mL, 3.00 eq) was added dropwise to the mixture via syringe. After 20 min, the mixture was allowed to warm to 25° C. for 6 h. The reaction mixture was quenched by addition ammonium chloride (10.0 mL) at 0° C. and extracted with ethyl acetate 20 mL (10 mL*2). The combined organic layers were dried over sodium sulfate, filtered and concentrated under reduced pressure to give a residue. The residue was purified by column chromatography (SiO2, petroleum ether/ethyl acetate=3/1) to afford (3-hydroxybicyclo[1.1.1]pentan-1-yl)(phenyl)methanone (160 mg, 850 umol, 69.0% yield) as light yellow oil.

1H NMR (400 MHz, CDCl3-d) δ=7.88 (d, J=7.6 Hz, 2H), 7.51-7.46 (m, 1H), 7.42-7.35 (m, 2H), 2.38 (s, 6H)

### Step 3. Procedure for Preparation of Compound 4—3-benzoylbicyclo[1.1.1]pentan-1-yl carbonochloridate

To a solution of (3-hydroxybicyclo[1.1.1]pentan-1-yl)(phenyl)methanone (60.0 mg, 319 umol, 1.00 eq) in dichloromethane (2.00 mL) was added triphosgene (151 mg, 510 umol, 1.60 eq) and N,N-diisopropylethylamine (82.4 mg, 638 umol, 111 uL, 2.00 eq) at 0° C. The mixture was stirred at 25° C. for 1 h. The reaction mixture was filtered and concentrated under reduced pressure to afford 3-benzoylbicyclo[1.1.1]pentan-1-yl carbonochloridate (70.0 mg, 279 umol, 88% yield) as a yellow solid.

### Step 4. Procedure for Preparation of Compound 4—1-(methyl(p-tolyl)carbamoyl)azetidin-3-yl (1-(4-(2,6-dioxopiperidin-3-yl)-3,5-difluorophenyl)azetidin-3-yl)carbamate

To a solution of 3-(4-(3-aminoazetidin-1-yl)-2,6-difluorophenyl)piperidine-2,6-dione (100 mg, 256 umol, 1.00 eq, methanesulfonic acid) in dichloromethane (3.00 mL) was added N,N-diisopropylethylamine (33.0 mg, 256 umol, 44.5 uL, 1.00 eq) and 3-benzoylbicyclo[1.1.1]pentan-1-yl carbonochloridate (70 mg, 279 umol, 1.09 eq). The mixture was stirred at 20° C. for 1 h. The reaction mixture was concentrated under reduced pressure to give a residue. The residue was purified by purified by reverse phase chromatography (C18, 40 g; condition: water/acetonitrile=100:0 to 0:100, 0.1% formic) and lyophilized to afford 1-(methyl(p-tolyl)carbamoyl)azetidin-3-yl (1-(4-(2,6-dioxopiperidin-3-yl)-3,5-difluorophenyl)azetidin-3-yl)carbamate (11.29 mg, 21.72 umol, 8.50% yield, 98% purity) as a white solid.

1H NMR (400 MHz, DMSO-d6) δ=10.86 (s, 1H), 8.03 (d, J=7.4 Hz, 1H), 7.96 (d, J=7.4 Hz, 2H), 7.69-7.62 (m, 1H), 7.58-7.51 (m, 2H), 6.16 (d, J=11.0 Hz, 2H), 4.50-4.36 (m, 1H), 4.11 (t, J=7.7 Hz, 2H), 4.04 (br dd, J=5.2, 12.8 Hz, 1H), 3.70-3.60 (m, 2H), 2.84-2.73 (m, 1H), 2.59 (s, 6H), 2.55-2.54 (m, 1H), 2.15-2.03 (m, 1H), 2.01-1.90 (m, 1H). MS (ESI) m/z 510.3 [M+H]+.

### Example 137. Synthesis of Compound 138

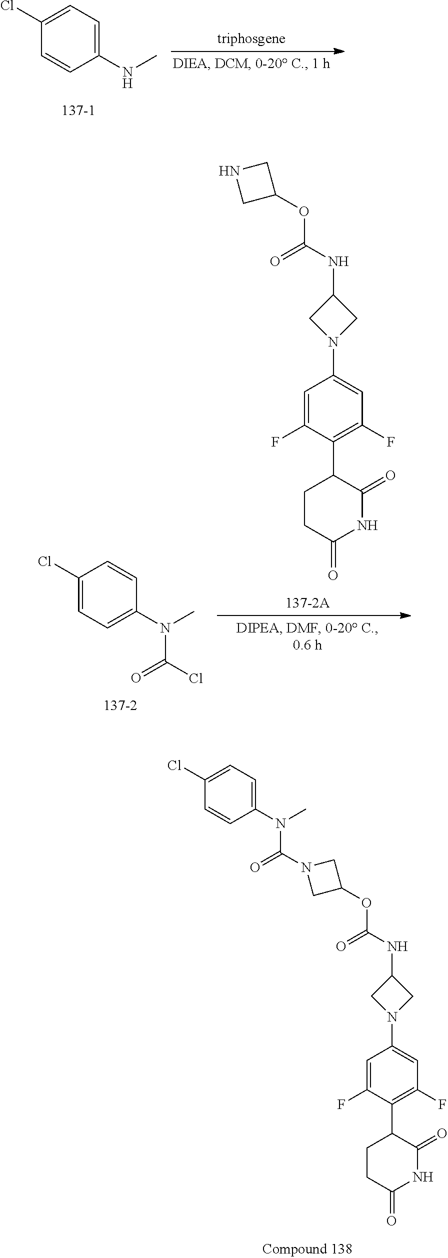

### Step 1. Procedure for Preparation of Compound 2—(4-chlorophenyl)(methyl)carbamic chloride

To a solution of 4-chloro-N-methylaniline (100 mg, 706 umol, 85.5 uL, 1.00 eq) in dichloromethane (1.00 mL) was added N,N-diisopropylethylamine (183 mg, 1.41 mmol, 246 μL, 2.00 eq) and triphosgene (210 mg, 706 umol, 1.00 eq) at 0° C. The mixture was stirred at 20° C. for 1 h. The reaction mixture was concentrated under reduced pressure to afford (4-chlorophenyl)(methyl)carbamic chloride (144 mg, 706 umol, 99% yield) as yellow oil.

### Step 2. Procedure for Preparation of 1-((4-chlorophenyl)(methyl)carbamoyl)azetidin-3-yl (1-(4-(2,6-dioxopiperidin-3-yl)-3,5-difluorophenyl)azetidin-3-yl)carbamate

To a solution of azetidin-3-yl (1-(4-(2,6-dioxopiperidin-3-yl)-3,5-difluorophenyl)azetidin-3-yl)carbamate (239 mg, 606 umol, 1.00 eq) in dichloromethane (3.00 mL) was added N,N-diisopropylethylamine (157 mg, 1.21 mmol, 211 uL, 2.00 eq) and (4-chlorophenyl)(methyl)carbamic chloride (136 mg, 667 umol, 1.10 eq) at 0° C. The mixture was stirred at 20° C. for 0.5 h. The reaction was filtered. The filtrate was purified by reversed-phase HPLC (C18, 80 g; condition: water/acetonitrile=1/0 to 0/1, 0.1% formic acid) and lyophilized to afford 1-((4-chlorophenyl)(methyl)carbamoyl)azetidin-3-yl (1-(4-(2,6-dioxopiperidin-3-yl)-3,5-difluorophenyl)azetidin-3-yl)carbamate (59.83 mg, 105.4 umol, 17% yield, 99% purity) as an off-white solid.

1H NMR (400 MHz, DMSO-d6) δ=10.85 (s, 1H), 8.05 (br d, J=7.0 Hz, 1H), 7.49-7.41 (m, 2H), 7.29 (d, J=8.6 Hz, 2H), 6.13 (d, J=11.0 Hz, 2H), 4.88-4.79 (m, 1H), 4.43-4.29 (m, 1H), 4.11-4.04 (m, 2H), 4.04-3.98 (m, 1H), 3.79 (br dd, J=6.9, 9.1 Hz, 2H), 3.60 (br t, J=6.8 Hz, 2H), 3.40-3.36 (m, 2H), 3.12 (s, 3H), 2.81-2.72 (m, 1H), 2.45-2.37 (m, 1H), 2.09-2.00 (m, 1H), 1.97-1.89 (m, 1H). MS (ESI) m/z 562.2 [M+H]⁺.

### Example 138. Synthesis of Compound 139

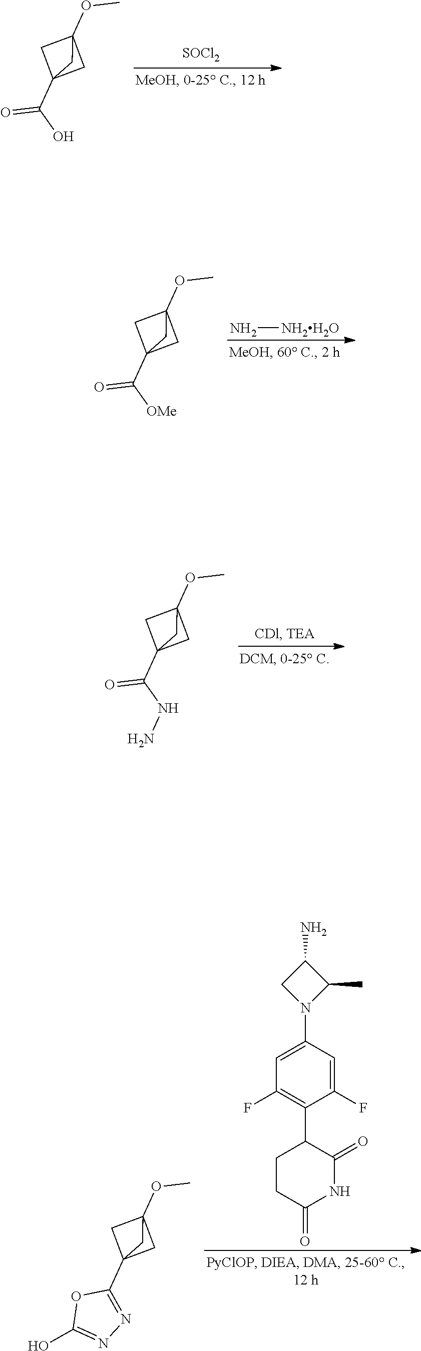

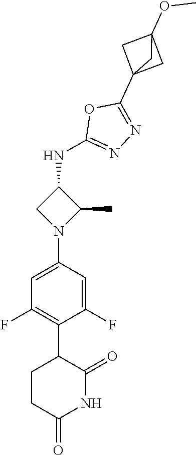 [SMILES: COC12CC(c3nnc(N[C@H]4CN(c5cc(F)c(C6CCC(=O)NC6=O)c(F)c5)[C@@H]4C)o3)(C1)C2]

### Step 1. Procedure for Preparation of methyl 3-methoxybicyclo[1.1.1]pentane-1-carboxylate

To a solution of 3-methoxybicyclo[1.1.1]pentane-1-carboxylic acid (500 mg, 3.52 mmol, 1.00 eq) in methanol (5.00 mL) was added thionyl chloride (837 mg, 7.03 mmol, 511 μL, 2.00 eq) at 0° C. The mixture was stirred at 25° C. for 12 hr. The reaction mixture was concentrated under reduced pressure to afford methyl 3-methoxybicyclo[1.1.1]pentane-1-carboxylate (548 mg, crude) as colorless liquid. ¹H NMR (400 MHz, DMSO-d₆) δ=3.61 (s, 3H), 3.20 (s, 3H), 2.10 (s, 6H)

### Step 2. Procedure for Preparation of 6-(trifluoromethoxy)nicotinohydrazide

To a solution of methyl 3-methoxybicyclo[1.1.1]pentane-1-carboxylate (548 mg, 3.51 mmol, 1.00 eq) in methanol (6.00 mL) was added hydrazine monohydrate (2.07 g, 35.1 mmol, 2.00 mL, 85% purity, 10.0 eq). The mixture was stirred at 60° C. for 2 hr. The reaction mixture was concentrated under reduced pressure to afford 3-methoxybicyclo[1.1.1]pentane-1-carbohydrazide (548 mg, crude) as a white solid.

### Step 3. Procedure for Preparation of 5-(3-methoxybicyclo[1.1.1]pentan-1-yl)-1,3,4-oxadiazol-2-ol

To a solution of 3-methoxybicyclo[1.1.1]pentane-1-carbohydrazide (548 mg, 3.51 mmol, 1.00 eq) in dichloromethane (5.00 mL) was added 1,1′-carbonyldiimidazole (683 mg, 4.21 mmol, 1.20 eq) at 0° C. and triethylamine (710 mg, 7.02 mmol, 977 μL, 2.00 eq). The mixture was stirred at 25° C. for 12 hr. The reaction mixture was concentrated under reduced pressure to give a residue. The residue was purified by prep-HPLC (formic acid condition; column: Phenomenex luna C18 150*40 mm*15 um; mobile phase: [water(formic acid)-acetonitrile]; gradient: 8%-38% B over 15 min) and lyophilized to afford 5-(3-methoxybicyclo[1.1.1]pentan-1-yl)-1,3,4-oxadiazol-2-ol (120 mg, 659 μmol, 19% yield) as brown oil. ¹H NMR (400 MHz, DMSO-d₆) δ=12.25 (br s, 1H), 3.23 (s, 3H), 2.20 (s, 6H)

### Step 4. Procedure for Preparation of 3-(2,6-difluoro-4-((2R,3S)-3-((5-(3-methoxybicyclo[1.1.1]pentan-1-yl)-, 3,4-oxadiazol-2-yl)amino)-2-methylazetidin-1-yl)phenyl)piperidine-2,6-dione

To a solution of 5-(3-methoxybicyclo[1.1.1]pentan-1-yl)-1,3,4-oxadiazol-2-ol (50.0 mg, 274 μmol, 1.00 eq) and 3-(4-((2R,3S)-3-amino-2-methylazetidin-1-yl)-2,6-difluorophenyl)piperidine-2,6-dione (84.9 mg, 274 μmol, 1.00 eq) in N,N-dimethylacetamide (1.00 mL) was added chloro(tripyrrolidin-1-yl)phosphonium;hexafluorophosphate (116 mg, 274 μmol, 1.00 eq) and N,N-diisopropylethylamine (70.9 mg, 549 μmol, 95.6 μL, 2.00 eq). The mixture was stirred at 25° C. for 0.5 hr. Then the reaction mixture was stirred at 60° C. for 11.5 h. The reaction mixture was concentrated under reduced pressure to give a residue. The residue was purified by prep-HPLC (neutral condition; column: Waters Xbridge 150*25 mm*5 um; mobile phase: [water(ammonium bicarbonate)-acetonitrile]; gradient: 25%-55% B over 9 min) and lyophilized to afford 3-(2,6-difluoro-4-((2R,3S)-3-((5-(3-methoxybicyclo[1.1.1]pentan-1-yl)-1,3,4-oxadiazol-2-yl)amino)-2-methylazetidin-1-yl)phenyl)piperidine-2,6-dione (11.83 mg, 24.74 μmol, 9.01% yield, 99% purity) as a white solid. ¹H NMR (400 MHz, DMSO-d₆) δ=10.87 (br s, 1H), 8.13 (d, J=7.2 Hz, 1H), 6.22 (d, J=11.2 Hz, 2H), 4.21 (t, J=7.4 Hz, 1H), 4.08-3.93 (m, 3H), 3.48 (br t, J=6.6 Hz, 1H), 3.24 (s, 3H), 2.85-2.71 (m, 1H), 2.54-2.51 (m, 1H), 2.21 (s, 6H), 2.13-2.02 (m, 1H), 1.99-1.90 (m, 1H), 1.46 (d, J=5.8 Hz, 3H)

### Example 139. Synthesis of Compound 140

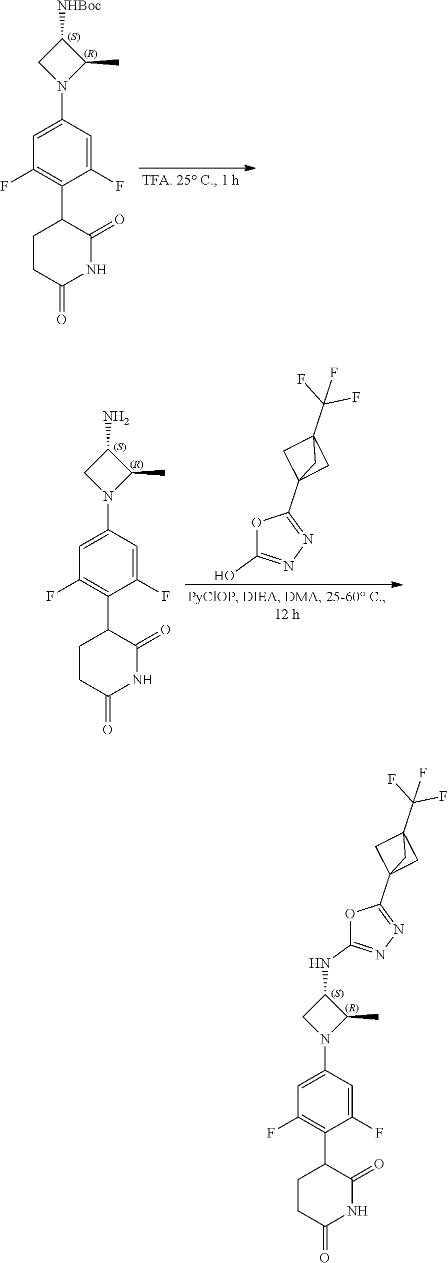

### Step 1. Procedure for preparation of 3-(4-((2R,3S)-3-amino-2-methylazetidin-1-yl)-2,6-difluorophenyl)piperidine-2,6-dione

To a solution of tert-butyl ((2R,3S)-1-(4-(2,6-dioxopiperidin-3-yl)-3,5-difluorophenyl)-2-methylazetidin-3-yl)carbamate (800 mg, 1.95 mmol, 1.00 eq) in trifluoroacetic anhydride (4.00 mL). The mixture was stirred at 25° C. for 1 h. The reaction mixture was filtered and concentrated under reduced pressure to give a residue. The residue was purified by prep-HPLC (column: Phenomenex luna C18 150*40 mm*15 um; mobile phase: [water(formic acid)-acetonitrile]; gradient: 0%-28% B over 15 min) to afford 3-(4-((2R,3S)-3-amino-2-methylazetidin-1-yl)-2,6-difluorophenyl)piperidine-2,6-dione (600 mg, 1.94 mmol, 99% yield) as a white solid. ¹H NMR (400 MHz, DMSO-d₆) δ=10.93 (s, 1H), 6.31 (d, J=11.0 Hz, 2H), 4.19 (br t, J=7.8 Hz, 1H), 4.11-4.05 (m, 2H), 3.70 (br d, J=6.6 Hz, 1H), 3.61-3.51 (m, 3H), 2.86-2.79 (m, 1H), 2.58 (s, 1H), 2.12 (br dd, J=3.2, 12.8 Hz, 1H), 2.01-1.95 (m, 1H), 1.47 (d, J=6.2 Hz, 3H)

### Step 2. Procedure for Preparation of 3-(2,6-difluoro-4-((2R,3S)-2-methyl-3-((5-(3-(trifluoromethyl)bicyclo[1.1.1]pentan-1-yl)-1,3,4-oxadiazol-2-yl)amino)azetidin-1-yl)phenyl)piperidine-2,6-dion

To a solution of 3-(4-((2R,3S)-3-amino-2-methylazetidin-1-yl)-2,6-difluorophenyl)piperidine-2,6-dione (95.0 mg, 307 μmol, 1.00 eq) and 5-(3-(trifluoromethyl)bicyclo[1.1.1]pentan-1-yl)-1,3,4-oxadiazol-2-ol (60.9 mg, 276 μmol, 0.900 eq) in N,N-dimethylacetamide (1.50 mL) were added N,N-diisopropylethylamine (198 mg, 1.54 mmol, 267 μL, 5.00 eq) and chloro(tripyrrolidin-1-yl)phosphonium;hexafluorophosphate (194 mg, 461 μmol, 1.50 eq) at 25° C. After 1 h, the mixture was stirred at 60° C. for 11 h. The reaction mixture was filtered and concentrated under reduced pressure to give a residue. The residue was purified by prep-HPLC (column: Waters Xbridge 150*25 mm*5 um; mobile phase: [water(ammoniumhydrogencarbonate)-acetonitrile]; gradient: 26%-56% B over 9 min) to afford 3-(2,6-difluoro-4-((2R,3S)-2-methyl-3-((5-(3-(trifluoromethyl)bicyclo[1.1.1]pentan-1-yl)-1,3,4-oxadiazol-2-yl)amino)azetidin-1-yl)phenyl)piperidine-2,6-dione (11.01 mg, 21.31 μmol, 6.94% yield, 99% purity) as a white solid. ¹H NMR (400 MHz, DMSO-d₆) δ=10.87 (br s, 1H), 8.23 (d, J=7.2 Hz, 1H), 6.23 (d, J=11.2 Hz, 2H), 4.22 (t, J=7.4 Hz, 1H), 4.09-4.02 (m, 2H), 4.00-3.96 (m, 1H), 3.49 (br s, 1H), 2.83-2.76 (m, 1H), 2.55 (br s, 1H), 2.40 (s, 6H), 2.14-2.05 (m, 1H), 1.99-1.93 (m, 1H), 1.47 (d, J=6.0 Hz, 3H)

### Example 140. Synthesis of Compound 141

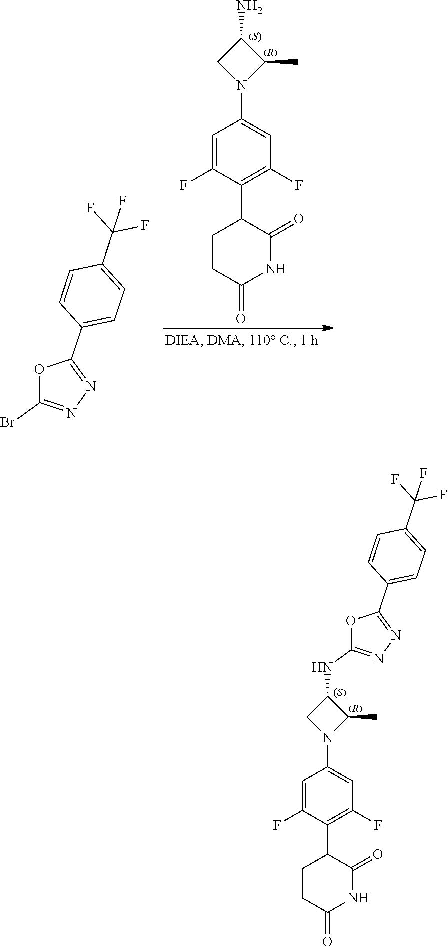

To a solution of 2-bromo-5-(4-(trifluoromethyl)phenyl)-1,3,4-oxadiazole (80.0 mg, 273 μmol, 1.00 eq) in N,N-dimethylacetamide (1.00 mL) was added N,N-diisopropylethylamine (70.6 mg, 546 μmol, 95.1 μL, 2.00 eq) and 3-(4-((2R,3S)-3-amino-2-methylazetidin-1-yl)-2,6-difluorophenyl)piperidine-2,6-dione (84.4 mg, 273 μmol, 1.00 eq). The mixture was stirred at 110° C. for 1 hr. The reaction mixture was concentrated under reduced pressure to give a residue. The residue was purified by prep-HPLC (formic acid condition; column: Phenomenex luna C18 150*25 mm*10 um; mobile phase: [water(formic acid)-acetonitrile]; gradient: 45%-75% B over 9 min) and lyophilized to afford 3-(2,6-difluoro-4-((2R,3S)-2-methyl-3-((5-(4-(trifluoromethyl)phenyl)-1,3,4-oxadiazol-2-yl)amino)azetidin-1-yl)phenyl)piperidine-2,6-dione e (67.95 mg, 129.01 μmol, 47.26% yield, 99% purity) was obtained as a white solid. ¹H NMR (400 MHz, DMSO-d₆) δ=10.88 (s, 1H), 8.57 (d, J=7.4 Hz, 1H), 8.04-7.98 (m, 2H), 7.91 (d, J=8.6 Hz, 2H), 6.25 (d, J=11.0 Hz, 2H), 4.33-4.23 (m, 1H), 4.21-4.11 (m, 1H), 4.10-4.01 (m, 2H), 3.65-3.53 (m, 1H), 2.86-2.72 (m, 1H), 2.52 (br d, J=3.2 Hz, 1H), 2.16-2.02 (m, 1H), 2.01-1.90 (m, 1H), 1.52 (d, J=6.0 Hz, 3H)

### Example 141. Synthesis of Compound 142

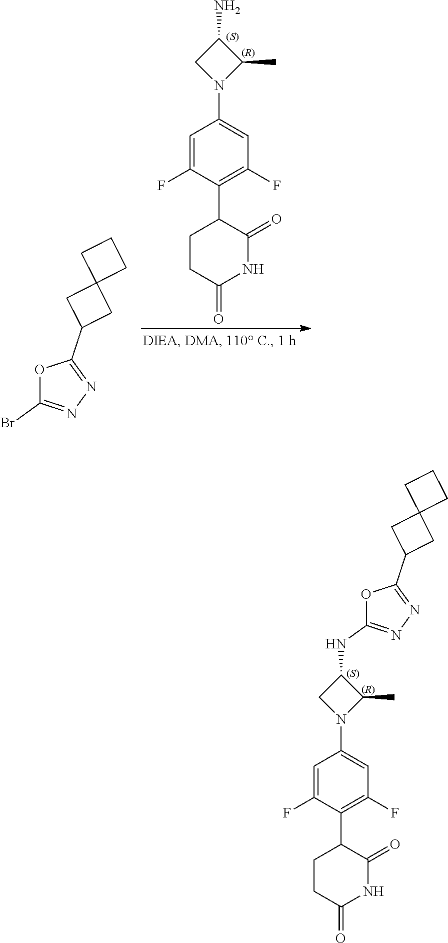

To a solution of 2-bromo-5-(spiro[3.3]heptan-2-yl)-1,3,4-oxadiazole (62.9 mg, 259 μmol, 1.00 eq) in N,N-dimethylacetamide (1.00 mL) were added N,N-diisopropylethylamine (100 mg, 776 μmol, 135 μL, 3.00 eq) and 3-(4-((2R,3S)-3-amino-2-methylazetidin-1-yl)-2,6-difluorophenyl)piperidine-2,6-dione (80.0 mg, 259 μmol, 1.00 eq). The mixture was stirred at 110° C. for 1 h. The reaction mixture was concentrated under reduced pressure to give a residue. The residue was purified by Prep-HPLC (column: Waters Xbridge 150*25 mm*5 um; mobile phase: [water(ammonium bicarbonate)-acetonitrile]; gradient: 38%-68% B over 9 min) and lyophilized to afford 3-(2,6-difluoro-4-((2R,3S)-2-methyl-3-((5-(spiro[3.3]heptan-2-yl)-1,3,4-oxadiazol-2-yl)amino)azetidin-1-yl)phenyl)piperidine-2,6-dione (24.07 mg, 50.03 μmol, 19.3% yield, 98% purity) as an off-white solid. ¹H NMR (400 MHz, DMSO-d₆) δ=10.88 (s, 1H), 8.01 (d, J=7.0 Hz, 1H), 6.23 (d, J=11.0 Hz, 2H), 4.27-4.16 (m, 1H), 4.09-3.95 (m, 3H), 3.54-3.46 (m, 1H), 3.42-3.37 (m, 1H), 2.84-2.74 (m, 1H), 2.49 (br s, 1H), 2.38-2.32 (m, 2H), 2.24-2.15 (m, 2H), 2.12-2.03 (m, 3H), 1.99-1.89 (m, 3H), 1.82-1.74 (m, 2H), 1.52-1.43 (m, 3H).

### Example 142. Synthesis of Compound 143

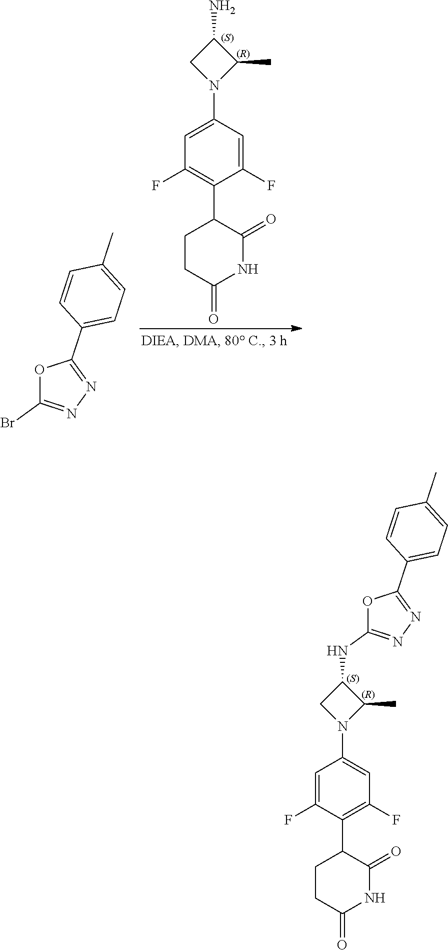

To a solution of 2-bromo-5-(p-tolyl)-1,3,4-oxadiazole (61.8 mg, 259 μmol, 1.00 eq) and 3-(4-((2R,3S)-3-amino-2-methylazetidin-1-yl)-2,6-difluorophenyl)piperidine-2,6-dione (80.0 mg, 259 μmol, 1.00 eq) in dimethylformamide (1.00 mL) were added N,N-diisopropylethylamine (83.6 mg, 647 μmol, 113 μL, 2.50 eq). The mixture was stirred at 80° C. for 3 h. The reaction mixture was filtered and concentrated under reduced pressure to give a residue. The residue was purified by prep-HPLC (column: Phenomenex luna C18 150*25 mm*10 um; mobile phase: [water(formic acid)-acetonitrile]; gradient: 40%-70% B over 9 min) to afford 3-(2,6-difluoro-4-((2R,3S)-2-methyl-3-((5-(p-tolyl)-1,3,4-oxadiazol-2-yl)amino)azetidin-1-yl)phenyl)piperidine-2,6-dione (33.7 mg, 71.4 μmol, 28% yield, 99% purity) as a white solid. ¹H NMR (400 MHz, DMSO-d₆) δ=10.89 (s, 1H), 8.36 (d, J=7.2 Hz, 1H), 7.70 (d, J=8.0 Hz, 2H), 7.35 (br d, J=8.0 Hz, 2H), 6.25 (br d, J=11.2 Hz, 2H), 4.30-4.24 (m, 1H), 4.17-4.11 (m, 1H), 4.09-4.03 (m, 2H), 3.57 (br t, J=6.8 Hz, 1H), 2.84-2.75 (m, 1H), 2.53 (br s, 1H), 2.37 (s, 3H), 2.15-2.04 (m, 1H), 2.01-1.93 (m, 1H), 1.52 (d, J=6.0 Hz, 3H)

### Example 143. Synthesis of Compound 144

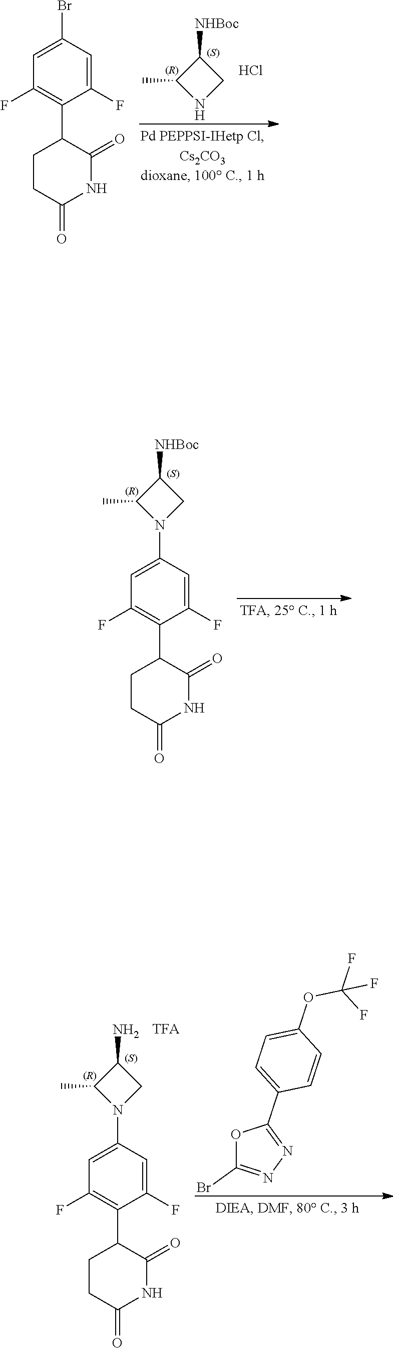

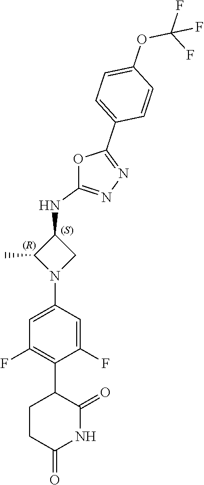

### Step 1. Procedure for Preparation of tert-butyl ((2R,3S)-1-(4-(2,6-dioxopiperidin-3-yl)-3,5-difluorophenyl)-2-methylazetidin-3-yl)carbamate

To a solution of 3-(4-bromo-2,6-difluorophenyl)piperidine-2,6-dione (1.37 g, 4.49 mmol, 1.00 eq) in dioxane (8.00 mL) was added tert-butyl ((2R,3S)-2-methylazetidin-3-yl)carbamate (1.00 g, 4.49 mmol, 1.00 eq, hydrochloride), cesium carbonate (4.39 g, 13.5 mmol, 3.00 eq) and 1,3-bis[2,6-bis(1-propylbutyl)phenyl]-4,5-dichloro-2H-imidazol-1-ium-2-ide;3-chloropyridine;dichloropalladium (218 mg, 225 μmol, 0.0500 eq) under nitrogen atmosphere. The mixture was stirred at 100° C. for 1 h under nitrogen atmosphere. Two batches were combined to filter and the filtrate was concentrated under reduced pressure to give a residue. The residue was purified by column chromatography (SiO₂, petroleum ether/ethyl acetate=5/1 to 1/1) to afford tert-butyl ((2R,3S)-1-(4-(2,6-dioxopiperidin-3-yl)-3,5-difluorophenyl)-2-methylazetidin-3-yl) carbamate (3.50 g, 7.61 mmol, 84% yield, 89% purity) as a yellow solid. ¹H NMR (400 MHz, DMSO-d₆) δ=10.85 (s, 1H), 7.41 (br d, J=7.0 Hz, 1H), 6.17 (d, J=11.1 Hz, 2H), 4.12-4.03 (m, 2H), 3.97-3.89 (m, 1H), 3.87-3.80 (m, 1H), 3.38 (br t, J=6.8 Hz, 1H), 2.84-2.72 (m, 1H), 2.52 (br s, 1H), 2.13-2.02 (m, 1H), 1.97-1.90 (m, 1H), 1.40 (br s, 3H), 1.38 (s, 9H).

### Step 2. Procedure for Preparation of 3-(4-((2R,3S)-3-amino-2-methylazetidin-1-yl)-2,6-difluorophenyl)piperidine-2,6-dione

A solution of tert-butyl ((2R,3S)-1-(4-(2,6-dioxopiperidin-3-yl)-3,5-difluorophenyl)-2-methylazetidin-3-yl) carbamate (1.50 g, 3.66 mmol, 1.00 eq) in trifluoroacetic acid (15.0 mL). The mixture was stirred at 25° C. for 1 h. The mixture was concentrated under reduced pressure to give a residue. The residue was purified by reverse phase chromatography (C18, 40 g; condition: water/acetonitrile=100/0 to 0/100, 0.1% formic acid) and lyophilized to afford 3-(4-((2R,3S)-3-amino-2-methylazetidin-1-yl)-2,6-difluorophenyl)piperidine-2,6-dione (1.10 g, 2.52 mmol, 68% yield, 97% purity, trifluoroacetate) as a white solid. ¹H NMR (400 MHz, DMSO-d₆) δ=10.87 (s, 1H), 8.27 (br s, 3H), 6.28 (d, J=11.0 Hz, 2H), 4.16 (t, J=8.0 Hz, 1H), 4.12-4.02 (m, 2H), 3.74-3.67 (m, 1H), 3.60-3.53 (m, 1H), 2.84-2.73 (m, 1H), 2.53 (br s, 1H), 2.10 (br d, J=3.1 Hz, 1H), 1.98-1.89 (m, 1H), 1.44 (d, J=5.9 Hz, 3H).

### Step 3. Procedure for Preparation of 3-(2,6-difluoro-4-((2R,3S)-2-methyl-3-((5-(4-(trifluoromethoxy)phenyl)-1,3,4-oxadiazol-2-yl)amino)azetidin-1-yl)phenyl)piperidine-2,6-dione

To a solution of 3-(4-((2R,3S)-3-amino-2-methylazetidin-1-yl)-2,6-difluorophenyl)piperidine-2,6-dione (1.10 g, 2.60 mmol, 1.00 eq, trifluoroacetate) in dimethylformamide (10.0 mL) was added N,N-diisopropylethylamine (1.01 g, 7.80 mmol, 1.36 mL, 3.00 eq) and 2-bromo-5-(4-(trifluoromethoxy)phenyl)-1,3,4-oxadiazole (883 mg, 2.86 mmol, 1.10 eq). The mixture was stirred at 80° C. for 3 h under nitrogen atmosphere. The mixture was concentrated under reduced pressure to give a residue. The residue was purified by column chromatography (SiO₂, petroleum ether/ethyl acetate=10/1 to 1/1) and lyophilized to afford 3-(2,6-difluoro-4-((2R,3S)-2-methyl-3-((5-(4-(trifluoromethoxy)phenyl)-1,3,4-oxadiazol-2-yl)amino)azetidin-1-yl)phenyl)piperidine-2,6-dione (1.032 g, 1.90 mmol, 73.16% yield, 99% purity) as a white solid. ¹H NMR (400 MHz, DMSO-d₆) δ=10.86 (s, 1H), 8.47 (d, J=7.1 Hz, 1H), 7.93 (d, J=8.8 Hz, 2H), 7.54 (d, J=8.4 Hz, 2H), 6.25 (d, J=11.1 Hz, 2H), 4.31-4.24 (m, 1H), 4.18-4.10 (m, 1H), 4.09-4.02 (m, 2H), 3.58 (br t, J=6.6 Hz, 1H), 2.84-2.72 (m, 1H), 2.53 (br s, 1H), 2.13-2.04 (m, 1H), 2.00-1.91 (m, 1H), 1.52 (d, J=6.1 Hz, 3H).

### Example 144. HTRF assay

Compound activity was monitored in a Homogenous Time-Resolved Fluorescence (HTRF) assay using 1-[5-({2-[2-(2-{[2-(2,6-dioxopiperidin-3-yl)-1,3-dioxo-2,3-dihydro-1H-isoindol-4-yl]oxy}acetamido)ethoxy]ethyl}carbamoyl)pentyl]-3,3-dimethyl-2-[(1E,3E)-5-[(2E)-1,3,3-trimethyl-5-sulfo-2,3-dihydro-1H-indol-2-ylidene]penta-1,3-dien-1-yl]-3H-indol-1-ium-5-sulfonate as a fluorescent probe. Biochemical assays were conducted in Greiner white 384 well HiBase plates (Cat. No 784075-25) in 10 μL total volume. A one pot detection solution of CRBN-DDB1 (2.5 nM), Anti-His Terbium Cryptate Gold (1×, PerkinElmer Cat. #: 61HI2TLB), and Cy5-Thalidomide (100 nM, Tenova Cat.: T52461) was prepared in 20 mM HEPES, 20 mM NaCl, 0.2 mM TCEP, 0.2 mM EDTA, and 0.005% Tween20 was dispensed to each assay plate. Compounds were stored in dry, ambient temperatures at 10 mM. A 10-point, 1:3 dilution series was prepared from 10 mM stock concentrations in Echo-compatible LDV plates. 10 nL of each compound dilution series was dispensed into assays wells using an Echo 650 (Labcyte inc. USA). 10 nL of 10 mM Lenalidomide was transferred into the active-control wells for the assay and 10 nL of DMSO was transferred into the neutral-control wells. The assay was then allowed to incubate for 30 min at ambient temperature after transferring compound. Plate measurements were taken on a Pherastar FSX (BMG Labtech, Germany) using the HTRF Red filter (Ex. 337 nm, em1: 620 nm, em2: 665 nm) (Flashes: 50, Integration time: 60-400 us, Z-height: 10 mm, Ratio-multiplier: 10,000). The HTRF signal was then subsequently normalized to the neutral and active controls. Analysis and IC50 values were derived using KNIME analytics (KNIME Zurich) transformation and fitting within Collaborative Drug Discovery (Collaborative Drug Discovery USA). Ki was derived from the geometric mean of the IC50 values using the Cheng-Prustoff transformation.

### Example 145. NanoBiT Assay

A HEK293 clonal line with a CRISPR knock-in C-terminal HiBiT tag on CDK2 and stably expressing LgBiT protein was obtained from Promega (Madison, WI). Cells were plated at 5000 cells per well using Multiflo (BioTek) in 384-well white solid bottom plates (Corning, 3570BC) in 25 ul volume in DMEM media (DMEM, high glucose, HEPES, no phenol red (ThermoFisher Scientific, 21063029)) containing 10% FBS (Corning, 35-075-CV), 1% Peniciliin/Streptomycin ((ThermoFisher Scientific, 15140-122), and 1% Endurazine (Nano-Glo Endurazine Live Cell Substrate (Promega, N2571)). Cells were incubated for 16 hours at 37 C, 5% CO2. 75 nL of a compound at 30 μM were dosed into the plate using an Echo® 650 liquid handler (Labcyte). Cells were incubated at 37° C., 5% CO2 for 24 hours and then signal was read on a Pherastar FSX using “LUM plus” optic module.

Analysis was performed in Scinamic (Scinamic, Cambridge, MA). Luminescence response (R) was calculated by the formula: response=100*(S−N)/(P−N) where S is the signal of the well, N and P the mean negative and positive control values respectively of the same plate. The luminescence response was then fitted in Scinamic using a 3-parameter agonist logistic fit (hillslope=1, EC50>0, top/bottom unconstrained).

### Table 2. HTRF and NanoBiT Data

For CDK2 NanoBiT: According to the code, A represents a Dₘₐₓ value of ≤10%, B represents a Dₘₐₓ value of >10% and ≤50%, C represents a Dₘₐₓ value of >50% and ≤80%, D represents Dₘₐₓ value of >80%. For HTRF: According to the code, E represents a Ki value of ≤0.1 μM, F represents a Ki value >0.1 μM and ≤1 μM, G represents an Ki value >1 μM.

| Compound No. | CDK2 NanoBiT Dmax (%) | CRBN HTRF Ki |
|---|---|---|
| 1 | D | E |
| 2 | C | F |
| 3 | D | E |
| 4 | D | E |
| 5 | C | E |
| 6 | C | E |
| 7 | B | E |
| 8 | D | E |
| 9 | D | E |
| 10 | D | E |
| 11 | C | E |
| 12 | C | E |
| 13 | C | E |
| 14 | B | E |
| 15 | C | E |
| 16 | B | E |
| 17 | C | E |
| 18 | B | E |
| 19 | B | E |
| 20 | B | E |
| 21 | B | E |
| 22 | C | F |
| 23 | C | E |
| 24 | B | E |
| 25 | B | E |
| 26 | C | E |
| 27 | B | E |
| 28 | D | E |
| 29 | C | E |
| 30 | C | E |
| 31 | C | E |
| 32 | C | E |
| 33 | C | E |
| 34 | C | F |
| 35 | D | F |
| 36 | C | E |
| 37 | C | F |
| 38 | D | E |
| 39 | C | E |
| 40 | C | E |
| 41 | B | E |
| 42 | B | E |
| 43 | C | E |
| 44 | D | F |
| 45 | B | E |
| 46 | C | E |
| 47 | D | E |
| 48 | D | E |
| 49 | D | F |
| 50 | D | F |
| 51 | D | E |
| 52 | D | E |
| 53 | D | E |
| 54 | C | E |
| 55 | C | F |
| 56 | D | E |
| 57 | C | F |
| 58 | C | E |
| 59 | D | E |
| 60 | D | E |
| 61 | D | F |
| 62 | D | F |
| 63 | D | F |
| 64 | D | E |
| 65 | D | E |
| 66 | D | E |
| 67 | D | E |
| 68 | D | F |
| 69 | C | E |
| 70 | C | E |
| 71 | C | E |
| 72 | D | E |
| 73 | C | E |
| 74 | C | E |
| 75 | C | E |
| 76 | B | E |
| 77 | B | E |
| 78 | C | E |
| 79 | C | E |
| 80 | C | E |
| 81 | D | E |
| 82 | D | E |
| 83 | C | E |
| 84 | B | E |
| 85 | C | — |
| 86 | B | F |
| 87 | C | F |
| 88 | C | E |
| 89 | C | G |
| 90 | D | F |
| 91 | D | F |
| 92 | C | E |
| 93 | D | F |
| 94 | D | F |
| 95 | D | E |
| 96 | D | E |
| 97 | D | F |
| 98 | D | E |
| 99 | D | F |
| 100 | C | E |
| 101 | C | E |
| 102 | D | E |
| 103 | D | E |
| 104 | D | E |
| 105 | C | F |
| 106 | C | E |
| 107 | D | E |
| 108 | D | E |
| 109 | B | E |
| 110 | B | E |
| 111 | C | E |
| 112 | B | E |
| 113 | B | E |
| 114 | B | E |
| 115 | B | E |
| 116 | B | E |
| 117 | B | E |
| 118 | — | E |
| 119 | — | E |
| 120 | B | E |
| 121 | B | E |
| 122 | B | E |
| 123 | B | E |
| 124 | B | E |
| 125 | C | E |
| 126 | B | E |
| 127 | B | E |
| 128 | C | E |
| 129 | C | E |
| 130 | C | E |
| 131 | C | E |
| 132 | C | E |
| 133 | C | E |
| 134 | C | E |
| 135 | C | F |
| 136 | C | E |
| 137 | C | E |
| 138 | C | E |
| 139 | C | F |
| 140 | C | F |
| 141 | C | F |
| 142 | C | E |
| 143 | C | F |
| 144 | C | E |

### EQUIVALENTS

While specific embodiments have been discussed, the above specification is illustrative and not restrictive. Many variations of the embodiments will become apparent to those skilled in the art upon review of this specification. The full scope of what is disclosed should be determined by reference to the claims, along with their full scope of equivalents, and the specification, along with such variations.

What is claimed is:

1. A compound of Formula (I): 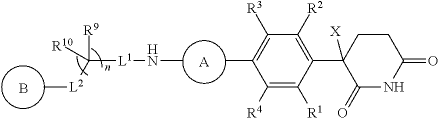
or a pharmaceutically acceptable salt thereof,
wherein:
X is selected from H and deuterium;
L¹ is selected from the group consisting of: 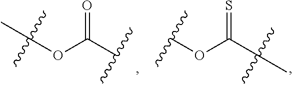 [SMILES: *OC(*)=O ; *OC(*)=S]
and 5-6 membered heteroaryl;
L² is selected from a bond and 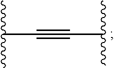 [SMILES: *C#C*]
each of R¹, R², R³, and R⁴ is independently selected from the group consisting of hydrogen, halogen, C₁₋₆alkoxy, cyano, hydroxy, C₃₋₆ cycloalkyl, and C₁₋₆alkyl;
ring A is selected from C₃₋₆ cycloalkyl and 3 to 6 membered heterocyclyl, wherein each of C₃₋₆cycloalkyl and 3 to 6 membered heterocyclyl is optionally substituted with one or more occurrences of R⁵;
each occurrence of R⁵ is independently selected from the group consisting of hydrogen, C₁₋₆ alkyl, hydroxy, and oxo, wherein C₁₋₆ alkyl is optionally substituted with one or more occurrences of halogen;
ring B is selected from the group consisting of C₃₋₁₂cycloalkyl, 3 to 10 membered heterocyclyl, aryl, and heteroaryl, wherein each of C₃₋₁₂cycloalkyl, 3 to 10 membered heterocyclyl, aryl, and heteroaryl is optionally substituted with one or more occurrences of R⁶;
each occurrence of R⁶ is independently selected from the group consisting of halogen, cyano, C₁₋₆alkoxy, C₁₋₆alkyl, —C(O)R⁷, —C(O)NR⁷R⁸, —S(O)₂R⁷, pyridine, 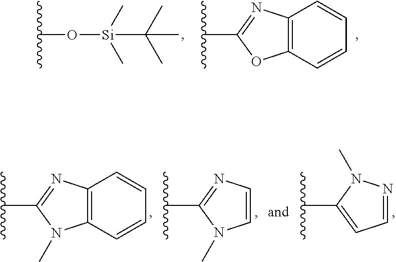
wherein each C₁₋₆alkyl, C₁₋₆ alkoxy, and pyridine is optionally substituted with one or more occurrences of a substitutent selected from C₁₋₆alkyl and halogen;
each occurrence of R⁷ is independently selected from the group consisting of C₁₋₆alkyl, phenyl, cyclopropane, an N-linked C₃₋₉ heterocycloalkyl, an N-linked heteroaryl, 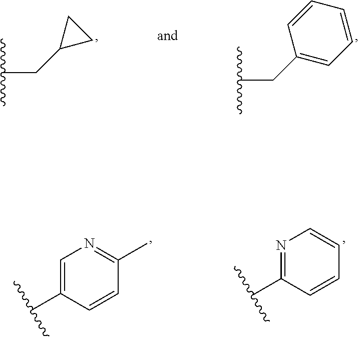
wherein R⁷ is optionally substituted with one or more occurrences of a substituent selected from the group consisting of C₁₋₆alkyl, halogen, cyano, trifluoro(methoxy)methane, and C₁₋₆alkoxy (e.g., methoxy);
each occurrence of R⁸, R⁹, and R¹⁰ is independently selected from hydrogen, deuterium, C₁₋₆alkyl, and deuterated C₁₋₆alkyl (e.g., —CD₃); and
n is an integer selected from the group consisting of 0, 1, 2, and 3.

2. The compound of claim 1, wherein ring A is selected from the group consisting of:

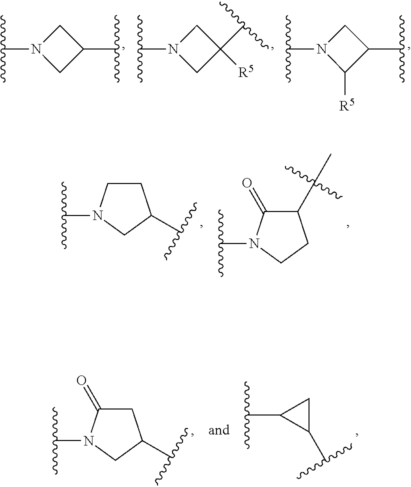

3. The compound of claim 1 or 2, ring B is selected from the group consisting of C₃₋₁₂ cycloalkyl, 3 to 10 membered heterocyclyl, and aryl, wherein each of C₃₋₁₂cycloalkyl, 3 to 10 membered heterocyclyl, and aryl is substituted with one or more occurrences of R₆.

4. The compound of claim 1 or 2, wherein ring B is selected from the group consisting of:

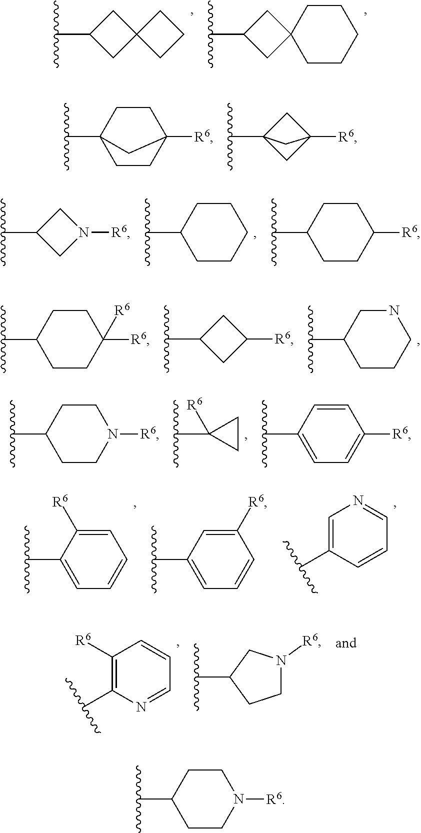

5. The compound of claim 1, wherein L¹ is selected from the group consisting of:

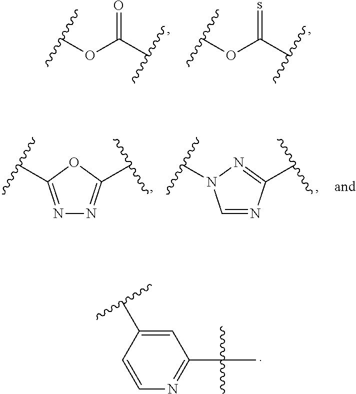

6. The compound of claim 1, wherein the compound is a compound of Formula (I-A):

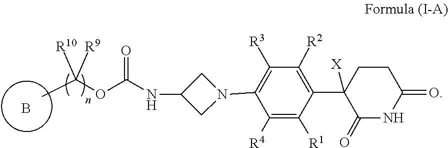

7. The compound of claim 1, wherein the compound is a compound of Formula (I-B):

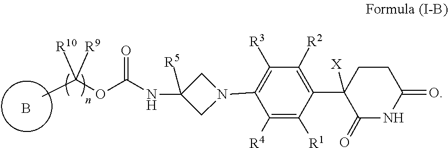

8. The compound of claim 1, wherein the compound is a compound of Formula (I-C):

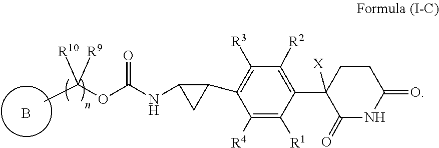

9. The compound of claim 1, wherein the compound is a compound of Formula (I-D):

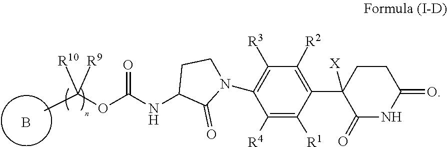

10. The compound of claim 1, wherein the compound is a compound of Formula (I-E):

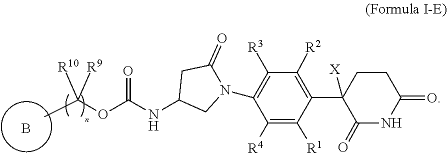

11. The compound of claim 1, wherein the compound is a compound of Formula (I-F):

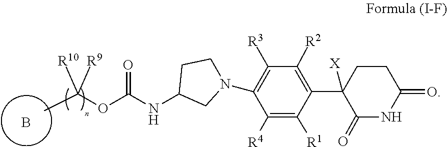

12. The compound of claim 1, wherein the compound is a compound of Formula (I-G):

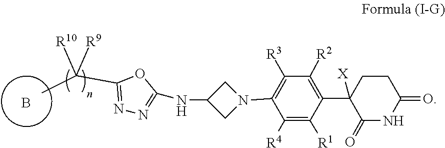

13. The compound of claim 1, wherein the compound is a compound of Formula (I-H):

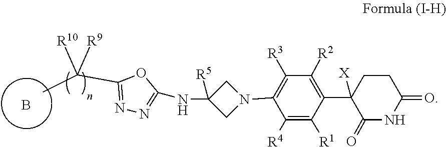

14. The compound of claim 1, wherein the compound is a compound of Formula (I-I):

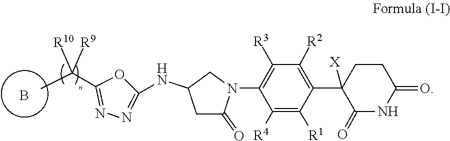

15. The compound of claim 1, wherein the compound is a compound of Formula (I-J):

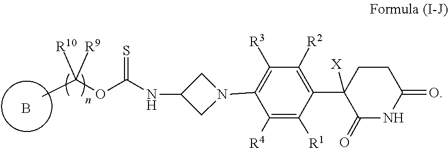

16. The compound of claim 1, wherein the compound is a compound of Formula (I-K):

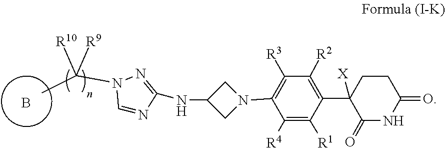

17. The compound of claim 1, wherein the compound is a compound of Formula (I-L):

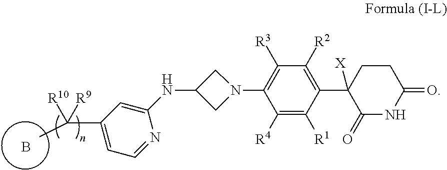

18. The compound of claim 1, wherein the compound is a compound of Formula (I-J-0):

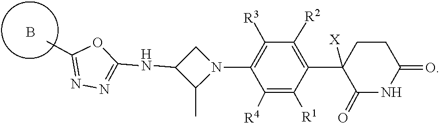

19. The compound of claim 1, wherein the compound is a compound of Formula (I-I-1-2):

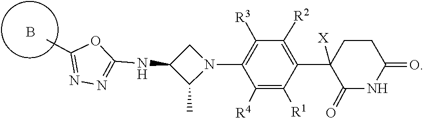

20. The compound of any one of claims 1-19, wherein X is H.

21. The compound of any one of claims 1-20, wherein L² is a bond.

22. The compound of any one of claims 1-21, wherein R¹, R², R³, and R⁴ are H.

23. The compound of any one of claims 1-21, wherein R¹ is fluoro, R² is fluoro, R³ is H, and R⁴ is H.

24. The compound of any one of claims 1-23, wherein R⁹ and R¹⁰ are H.

25. The compound of any one of claims 1-24, wherein n is 3.

26. The compound of any one of claims 1-24, wherein n is 1.

27. The compound of any one of claims 1-24, wherein n is 0.

28. The compound of any one of claims 1-27, wherein R⁶ is selected from the group consisting of Cl, F, —CN, —CH₃, —CF₃, —CH(CH₃)₂, —OCH₃, —OC(CH₃)₃, —OCF₃, —O—Si(CH₃)₂C(CH₃)₃, —C(O)R⁷, R⁶ is —C(O)NR⁷R⁸, and —S(O)₂R⁷.

29. The compound of any one of claims 1-28, wherein R⁷ is selected from the group consisting of methyl, benzene, cyclopropane 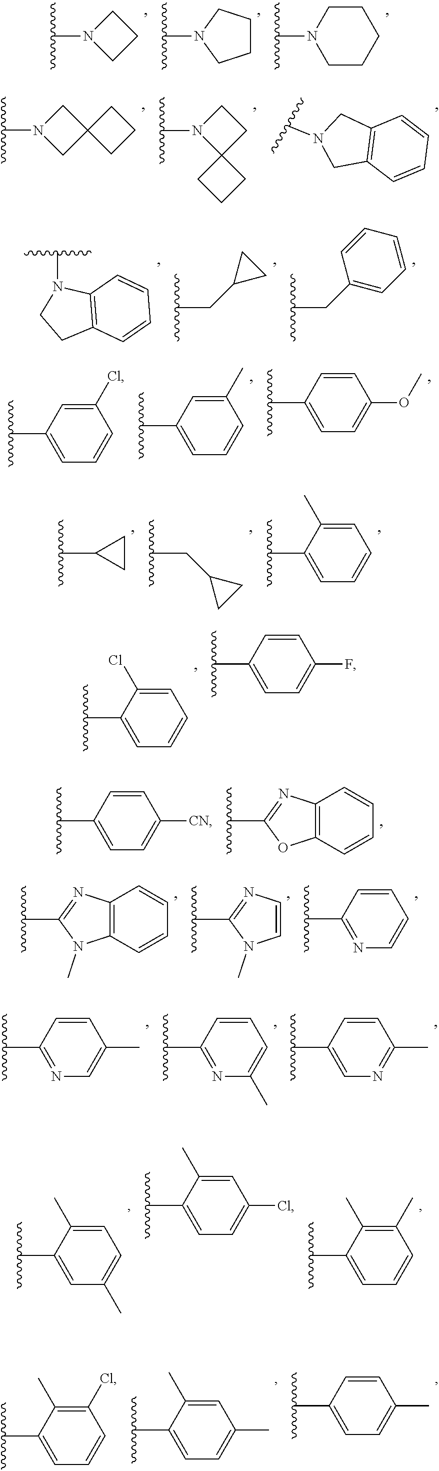 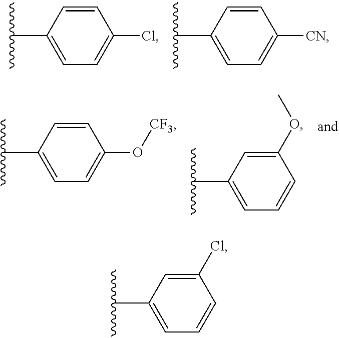
wherein each of 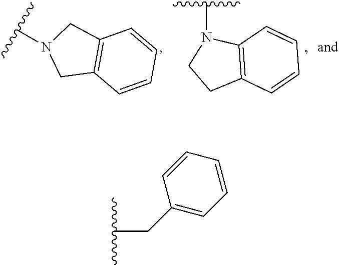
is optionally substituted with one or two occurrences selected from the group consisting of methyl, flourine, chlorine, cyano, and methoxy.

30. The compound of any one of claims 1-29, wherein each occurrence of R⁸, R⁹, and R¹⁰ is independently hydrogen, CH₃, or CD₃.

31. A compound selected from the group consisting of: 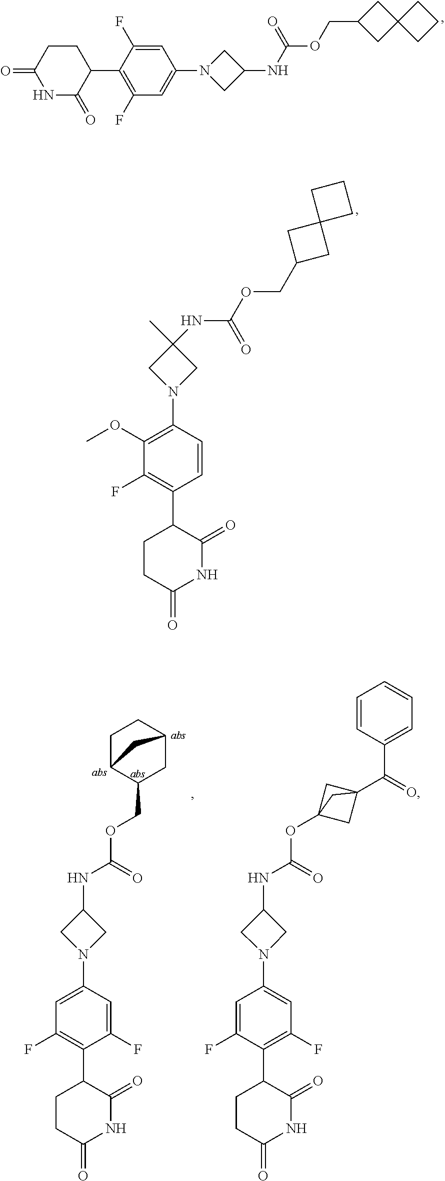 [SMILES: O=C1CCC(c2c(F)cc(N3CC(NC(=O)OCC4CC5(CCC5)C4)C3)cc2F)C(=O)N1 ; COc1c(N2CC(C)(NC(=O)OCC3CC4(CCC4)C3)C2)ccc(C2CCC(=O)NC2=O)c1F ; O=C1CCC(c2c(F)cc(N3CC(NC(=O)OC[C@@H]4C[C@H]5CC[C@@H]4C5)C3)cc2F)C(=O)N1 ; O=C1CCC(c2c(F)cc(N3CC(NC(=O)OC45CC(C(=O)c6ccccc6)(C4)C5)C3)cc2F)C(=O)N1] 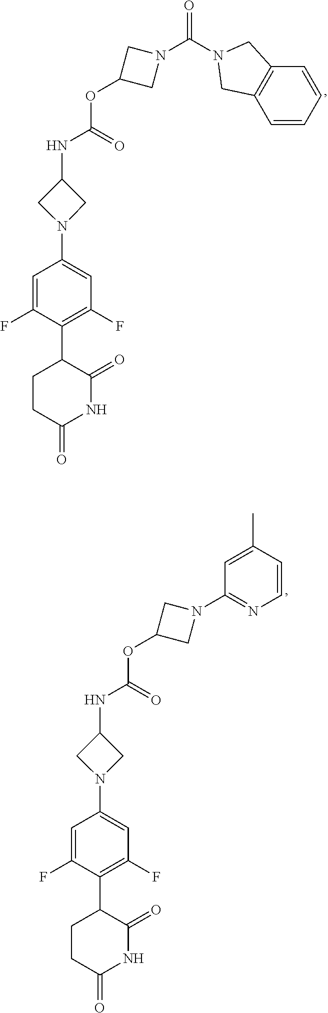 [SMILES: O=C1CCC(c2c(F)cc(N3CC(NC(=O)OC4CN(C(=O)N5Cc6ccccc6C5)C4)C3)cc2F)C(=O)N1 ; Cc1ccnc(N2CC(OC(=O)NC3CN(c4cc(F)c(C5CCC(=O)NC5=O)c(F)c4)C3)C2)c1] 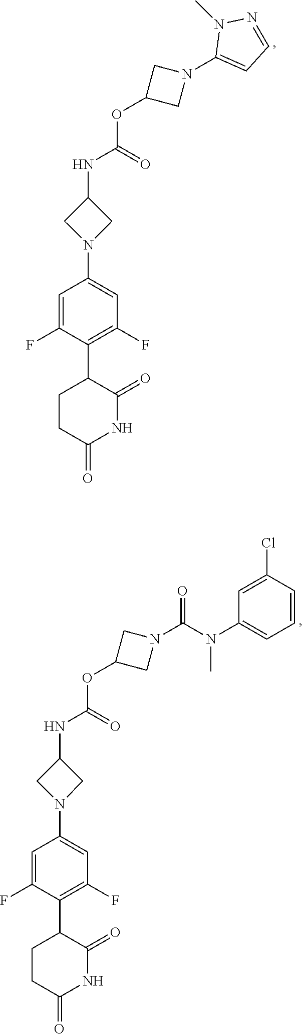 [SMILES: Cn1nccc1N1CC(OC(=O)NC2CN(c3cc(F)c(C4CCC(=O)NC4=O)c(F)c3)C2)C1 ; CN(C(=O)N1CC(OC(=O)NC2CN(c3cc(F)c(C4CCC(=O)NC4=O)c(F)c3)C2)C1)c1cccc(Cl)c1] 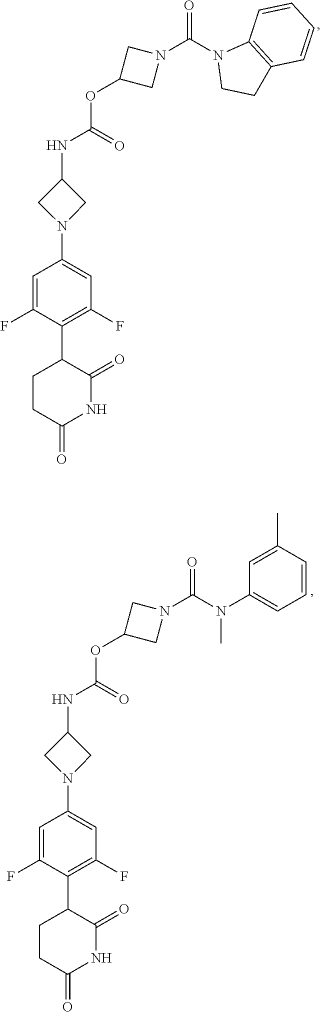 [SMILES: O=C1CCC(c2c(F)cc(N3CC(NC(=O)OC4CN(C(=O)N5CCc6ccccc65)C4)C3)cc2F)C(=O)N1 ; Cc1cccc(N(C)C(=O)N2CC(OC(=O)NC3CN(c4cc(F)c(C5CCC(=O)NC5=O)c(F)c4)C3)C2)c1] 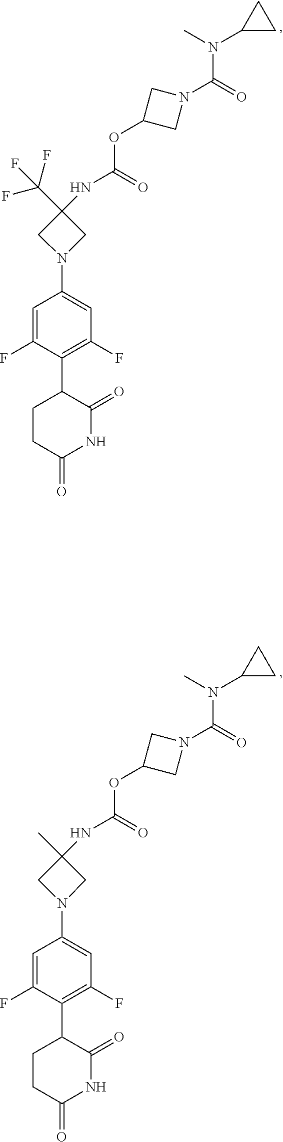 [SMILES: CN(C(=O)N1CC(OC(=O)NC2(C(F)(F)F)CN(c3cc(F)c(C4CCC(=O)NC4=O)c(F)c3)C2)C1)C1CC1 ; CN(C(=O)N1CC(OC(=O)NC2(C)CN(c3cc(F)c(C4CCC(=O)NC4=O)c(F)c3)C2)C1)C1CC1] 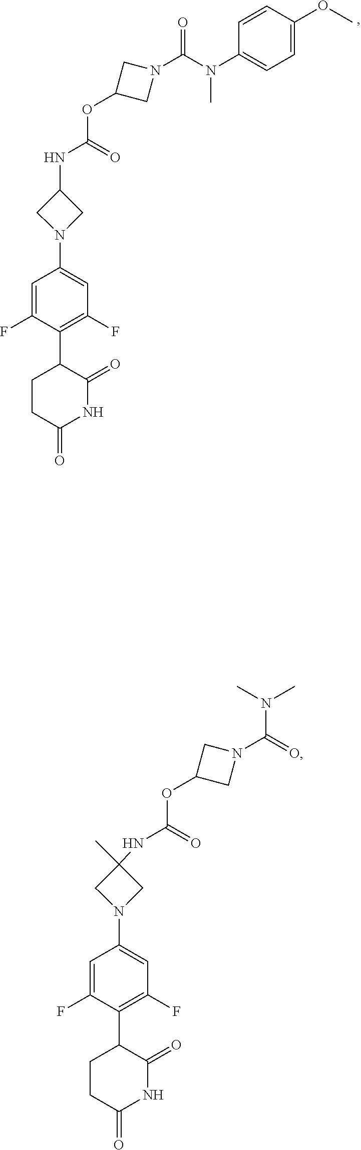 [SMILES: COc1ccc(N(C)C(=O)N2CC(OC(=O)NC3CN(c4cc(F)c(C5CCC(=O)NC5=O)c(F)c4)C3)C2)cc1 ; CN(C)C(=O)N1CC(OC(=O)NC2(C)CN(c3cc(F)c(C4CCC(=O)NC4=O)c(F)c3)C2)C1] 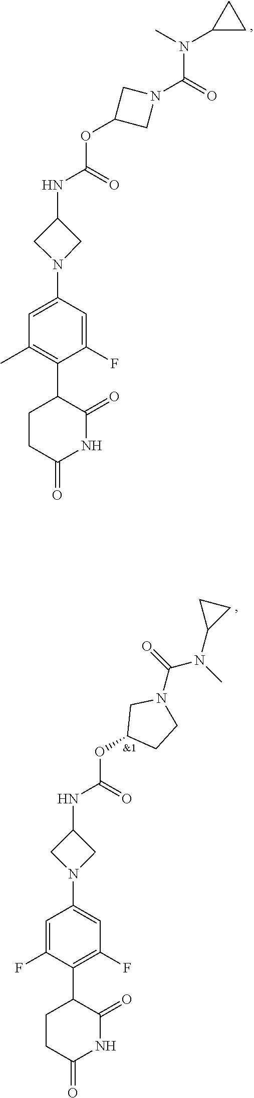 [SMILES: Cc1cc(N2CC(NC(=O)OC3CN(C(=O)N(C)C4CC4)C3)C2)cc(F)c1C1CCC(=O)NC1=O ; CN(C(=O)N1CC[C@H](OC(=O)NC2CN(c3cc(F)c(C4CCC(=O)NC4=O)c(F)c3)C2)C1)C1CC1] 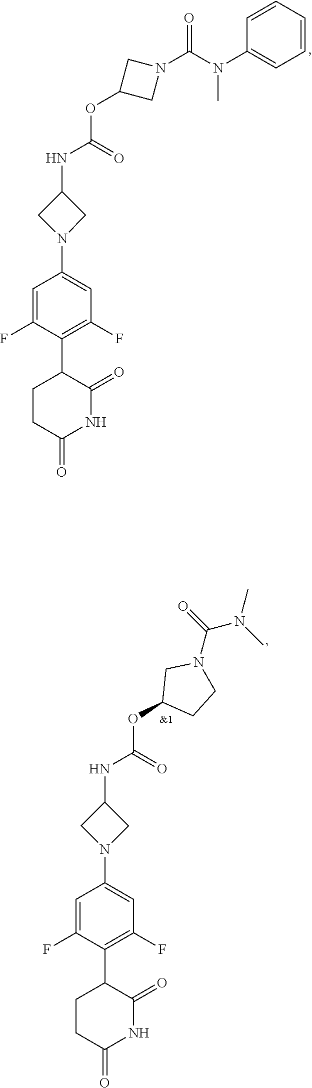 [SMILES: CN(C(=O)N1CC(OC(=O)NC2CN(c3cc(F)c(C4CCC(=O)NC4=O)c(F)c3)C2)C1)c1ccccc1 ; CN(C)C(=O)N1CC[C@@H](OC(=O)NC2CN(c3cc(F)c(C4CCC(=O)NC4=O)c(F)c3)C2)C1] 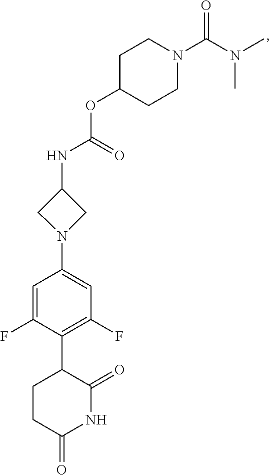 [SMILES: CN(C)C(=O)N1CCC(OC(=O)NC2CN(c3cc(F)c(C4CCC(=O)NC4=O)c(F)c3)C2)CC1] 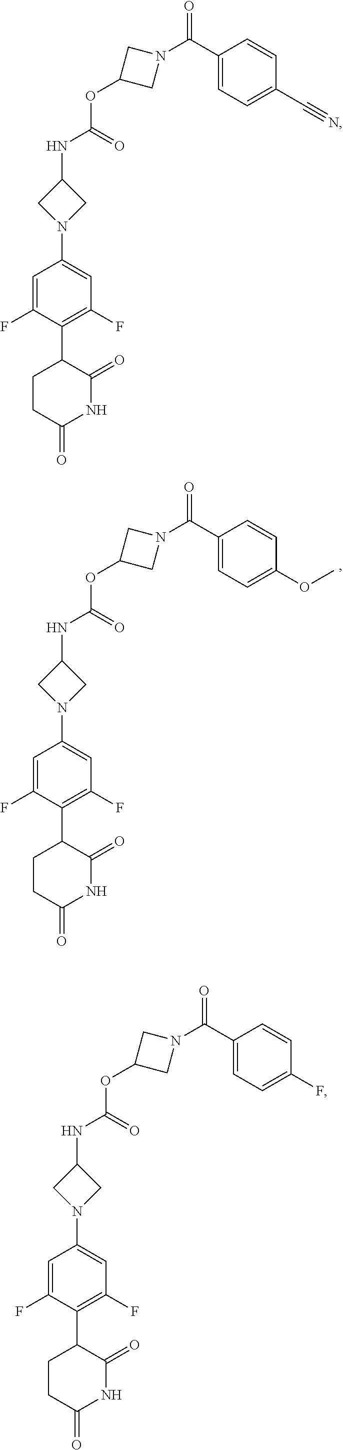 [SMILES: N#Cc1ccc(C(=O)N2CC(OC(=O)NC3CN(c4cc(F)c(C5CCC(=O)NC5=O)c(F)c4)C3)C2)cc1 ; COc1ccc(C(=O)N2CC(OC(=O)NC3CN(c4cc(F)c(C5CCC(=O)NC5=O)c(F)c4)C3)C2)cc1 ; O=C1CCC(c2c(F)cc(N3CC(NC(=O)OC4CN(C(=O)c5ccc(F)cc5)C4)C3)cc2F)C(=O)N1] 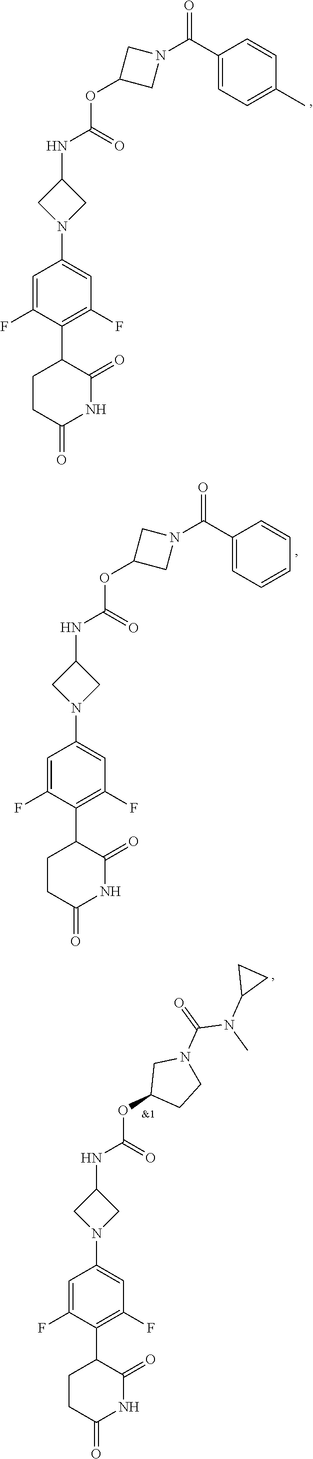 [SMILES: Cc1ccc(C(=O)N2CC(OC(=O)NC3CN(c4cc(F)c(C5CCC(=O)NC5=O)c(F)c4)C3)C2)cc1 ; O=C1CCC(c2c(F)cc(N3CC(NC(=O)OC4CN(C(=O)c5ccccc5)C4)C3)cc2F)C(=O)N1 ; CN(C(=O)N1CC[C@@H](OC(=O)NC2CN(c3cc(F)c(C4CCC(=O)NC4=O)c(F)c3)C2)C1)C1CC1] 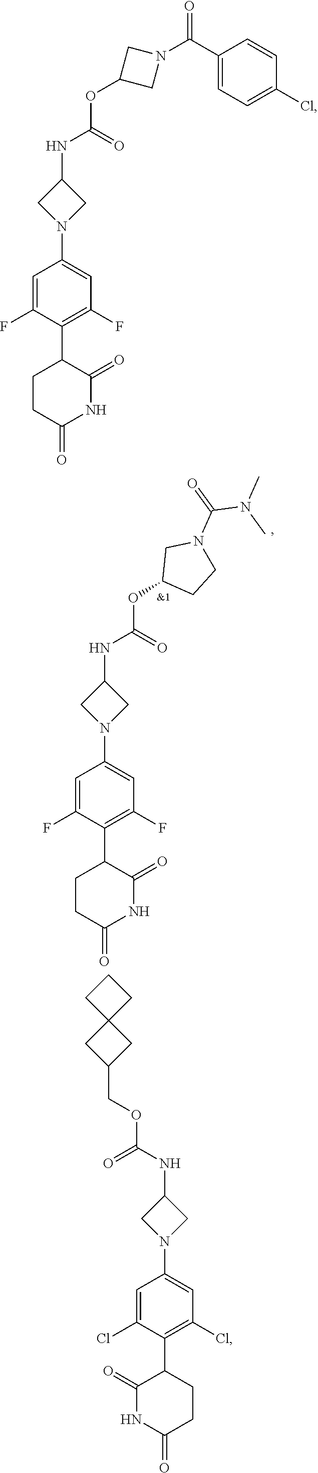 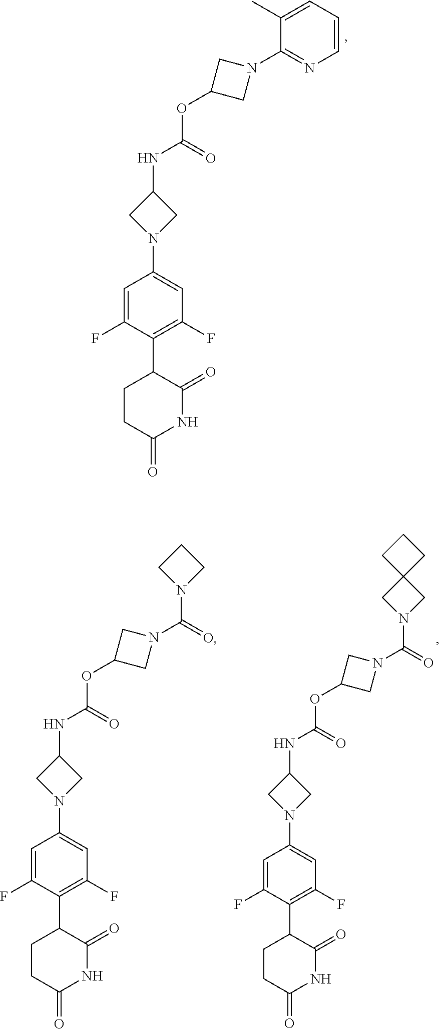 [SMILES: Cc1cccnc1N1CC(OC(=O)NC2CN(c3cc(F)c(C4CCC(=O)NC4=O)c(F)c3)C2)C1 ; O=C1CCC(c2c(F)cc(N3CC(NC(=O)OC4CN(C(=O)N5CCC5)C4)C3)cc2F)C(=O)N1 ; O=C1CCC(c2c(F)cc(N3CC(NC(=O)OC4CN(C(=O)N5CC6(CCC6)C5)C4)C3)cc2F)C(=O)N1] 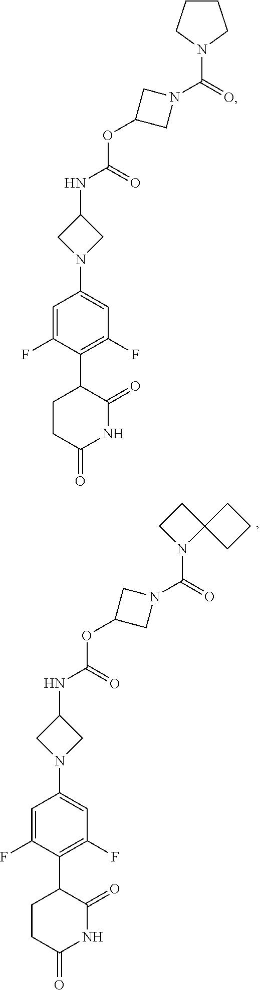 [SMILES: O=C1CCC(c2c(F)cc(N3CC(NC(=O)OC4CN(C(=O)N5CCCC5)C4)C3)cc2F)C(=O)N1 ; O=C1CCC(c2c(F)cc(N3CC(NC(=O)OC4CN(C(=O)N5CCC56CCC6)C4)C3)cc2F)C(=O)N1] 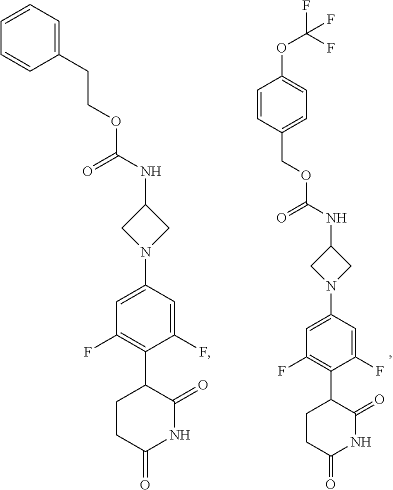 [SMILES: O=C1CCC(c2c(F)cc(N3CC(NC(=O)OCCc4ccccc4)C3)cc2F)C(=O)N1 ; O=C1CCC(c2c(F)cc(N3CC(NC(=O)OCc4ccc(OC(F)(F)F)cc4)C3)cc2F)C(=O)N1] 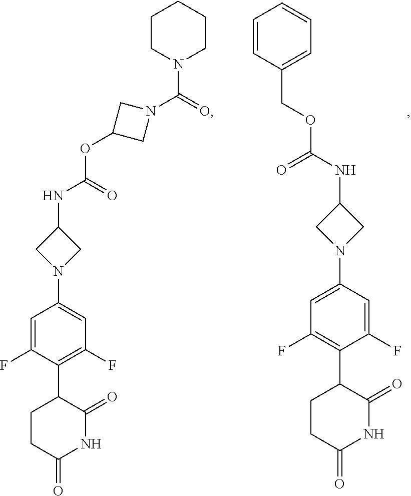 [SMILES: O=C1CCC(c2c(F)cc(N3CC(NC(=O)OC4CN(C(=O)N5CCCCC5)C4)C3)cc2F)C(=O)N1 ; O=C1CCC(c2c(F)cc(N3CC(NC(=O)OCc4ccccc4)C3)cc2F)C(=O)N1] 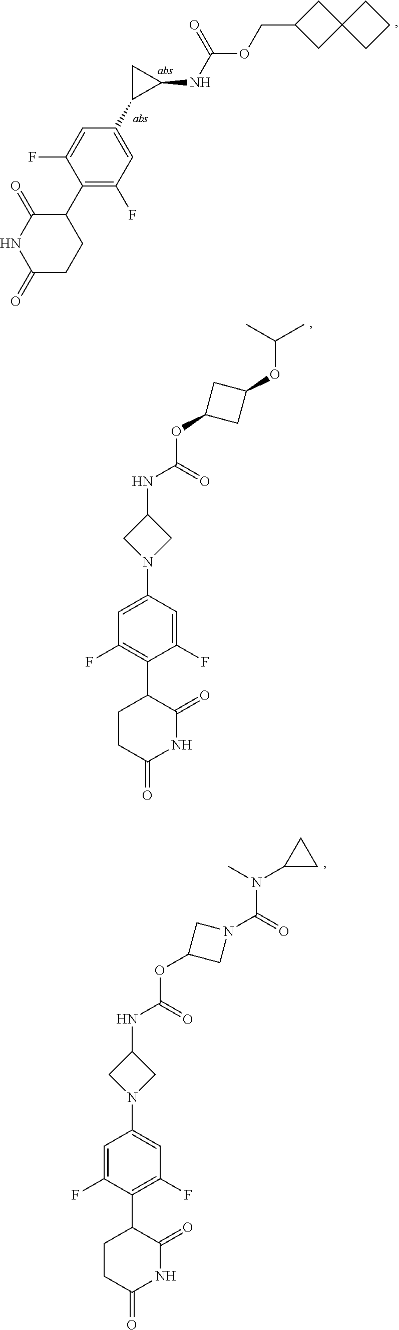 [SMILES: O=C1CCC(c2c(F)cc([C@@H]3C[C@H]3NC(=O)OCC3CC4(CCC4)C3)cc2F)C(=O)N1 ; CC(C)O[C@H]1C[C@@H](OC(=O)NC2CN(c3cc(F)c(C4CCC(=O)NC4=O)c(F)c3)C2)C1 ; CN(C(=O)N1CC(OC(=O)NC2CN(c3cc(F)c(C4CCC(=O)NC4=O)c(F)c3)C2)C1)C1CC1] 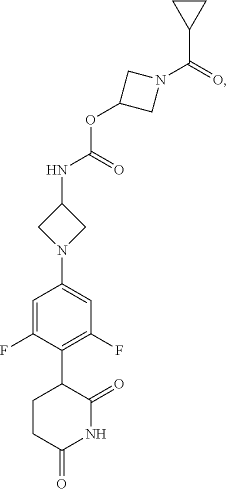 [SMILES: O=C1CCC(c2c(F)cc(N3CC(NC(=O)OC4CN(C(=O)C5CC5)C4)C3)cc2F)C(=O)N1] 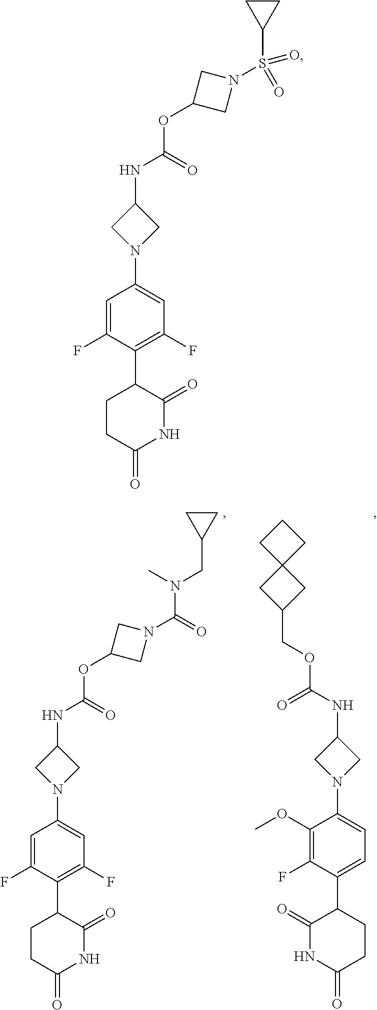 [SMILES: O=C1CCC(c2c(F)cc(N3CC(NC(=O)OC4CN(S(=O)(=O)C5CC5)C4)C3)cc2F)C(=O)N1 ; CN(CC1CC1)C(=O)N1CC(OC(=O)NC2CN(c3cc(F)c(C4CCC(=O)NC4=O)c(F)c3)C2)C1 ; COc1c(N2CC(NC(=O)OCC3CC4(CCC4)C3)C2)ccc(C2CCC(=O)NC2=O)c1F] 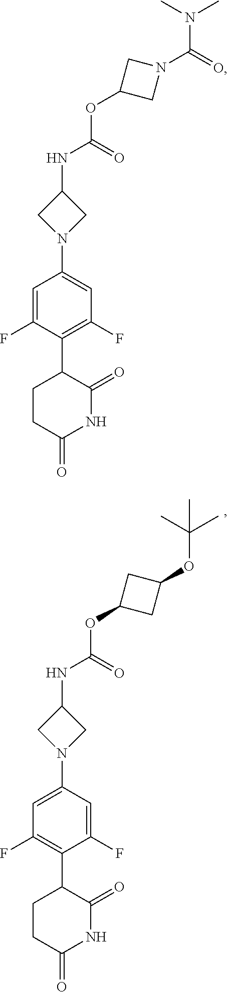 [SMILES: CN(C)C(=O)N1CC(OC(=O)NC2CN(c3cc(F)c(C4CCC(=O)NC4=O)c(F)c3)C2)C1 ; CC(C)(C)O[C@H]1C[C@@H](OC(=O)NC2CN(c3cc(F)c(C4CCC(=O)NC4=O)c(F)c3)C2)C1] 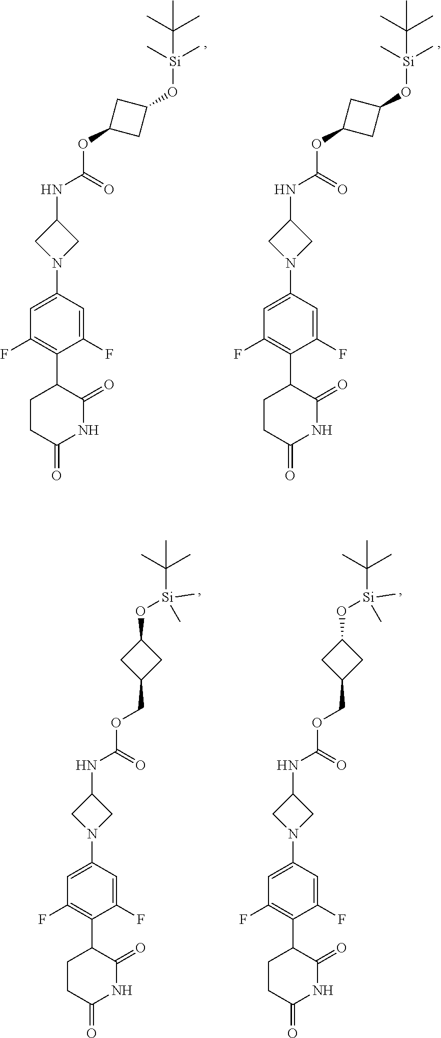 [SMILES: CC(C)(C)[Si](C)(C)O[C@H]1C[C@H](OC(=O)NC2CN(c3cc(F)c(C4CCC(=O)NC4=O)c(F)c3)C2)C1 ; CC(C)(C)[Si](C)(C)O[C@H]1C[C@@H](OC(=O)NC2CN(c3cc(F)c(C4CCC(=O)NC4=O)c(F)c3)C2)C1 ; CC(C)(C)[Si](C)(C)O[C@H]1C[C@@H](COC(=O)NC2CN(c3cc(F)c(C4CCC(=O)NC4=O)c(F)c3)C2)C1 ; CC(C)(C)[Si](C)(C)O[C@H]1C[C@H](COC(=O)NC2CN(c3cc(F)c(C4CCC(=O)NC4=O)c(F)c3)C2)C1] 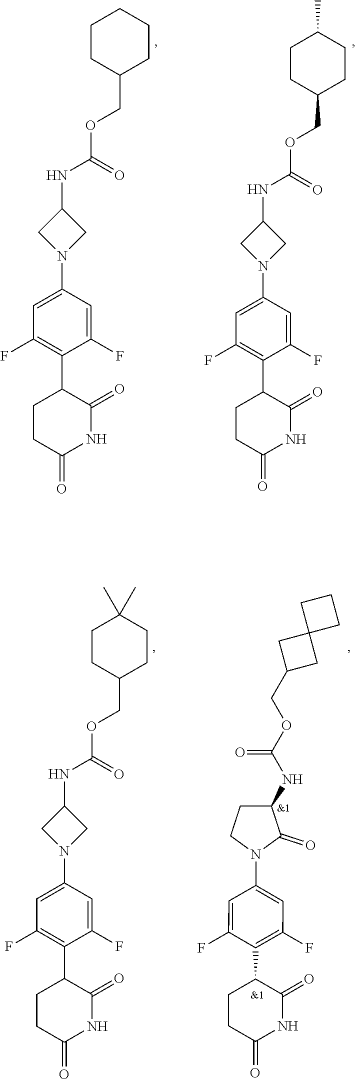 [SMILES: O=C1CCC(c2c(F)cc(N3CC(NC(=O)OCC4CCCCC4)C3)cc2F)C(=O)N1 ; C[C@H]1CC[C@H](COC(=O)NC2CN(c3cc(F)c(C4CCC(=O)NC4=O)c(F)c3)C2)CC1 ; CC1(C)CCC(COC(=O)NC2CN(c3cc(F)c(C4CCC(=O)NC4=O)c(F)c3)C2)CC1 ; O=C1CC[C@H](c2c(F)cc(N3CC[C@@H](NC(=O)OCC4CC5(CCC5)C4)C3=O)cc2F)C(=O)N1] 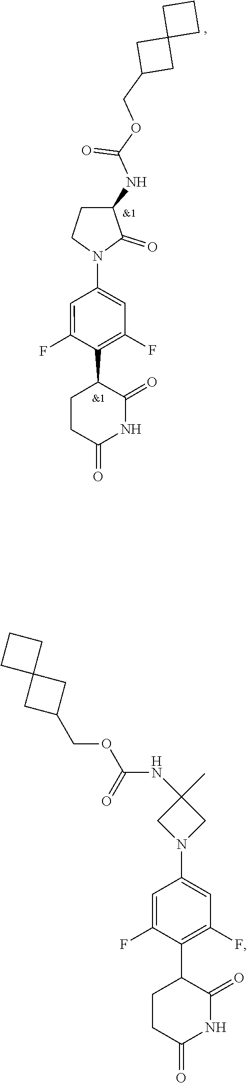 [SMILES: O=C1CC[C@@H](c2c(F)cc(N3CC[C@@H](NC(=O)OCC4CC5(CCC5)C4)C3=O)cc2F)C(=O)N1 ; CC1(NC(=O)OCC2CC3(CCC3)C2)CN(c2cc(F)c(C3CCC(=O)NC3=O)c(F)c2)C1] 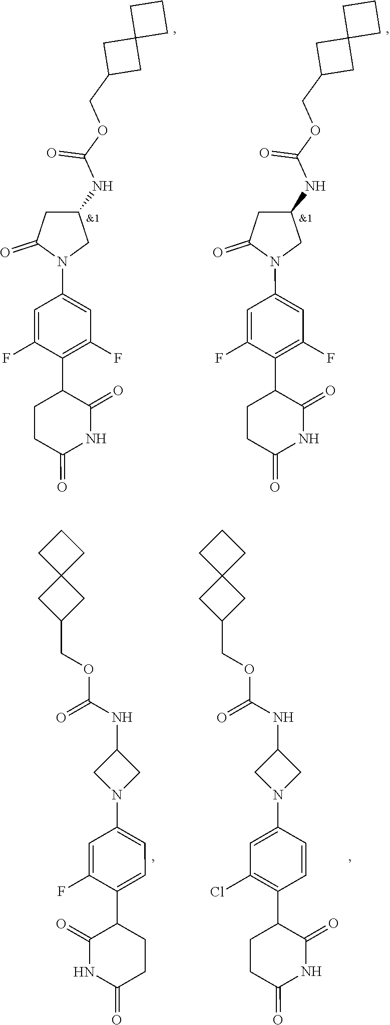 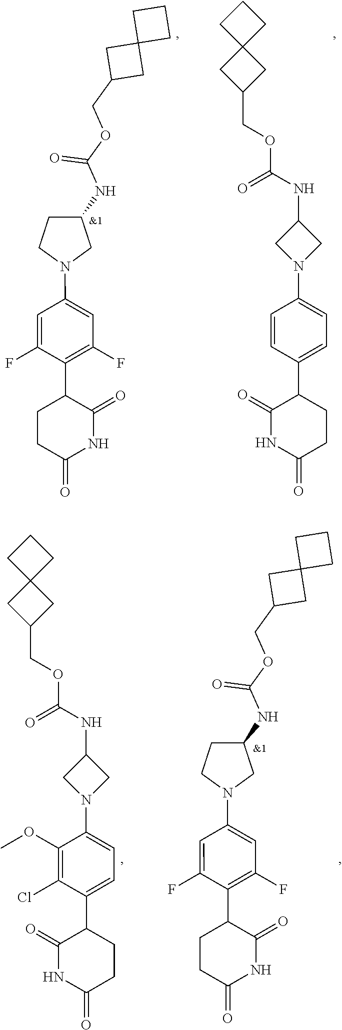 [SMILES: O=C1CCC(c2c(F)cc(N3CC[C@H](NC(=O)OCC4CC5(CCC5)C4)C3)cc2F)C(=O)N1 ; O=C1CCC(c2ccc(N3CC(NC(=O)OCC4CC5(CCC5)C4)C3)cc2)C(=O)N1 ; COc1c(N2CC(NC(=O)OCC3CC4(CCC4)C3)C2)ccc(C2CCC(=O)NC2=O)c1Cl ; O=C1CCC(c2c(F)cc(N3CC[C@@H](NC(=O)OCC4CC5(CCC5)C4)C3)cc2F)C(=O)N1] 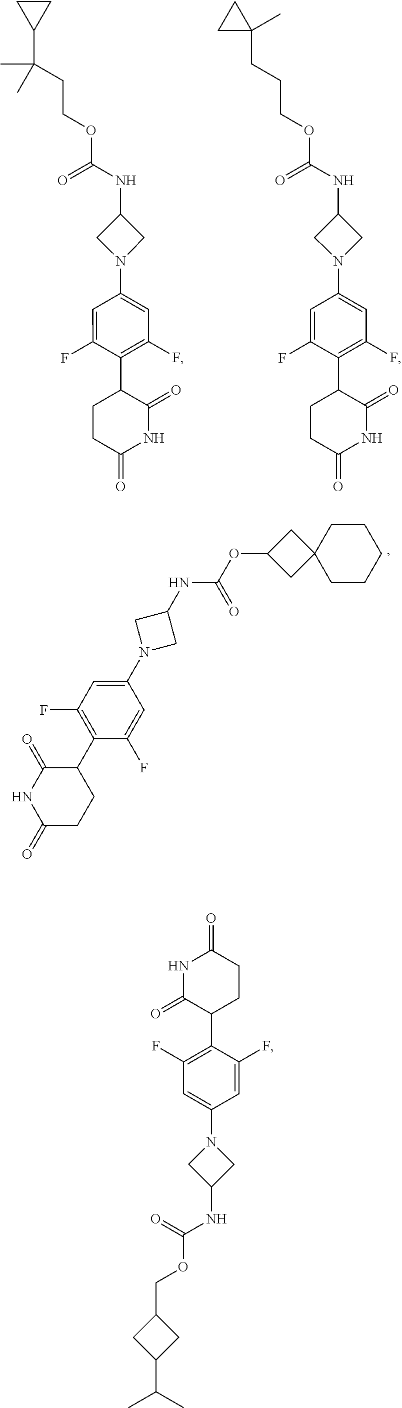 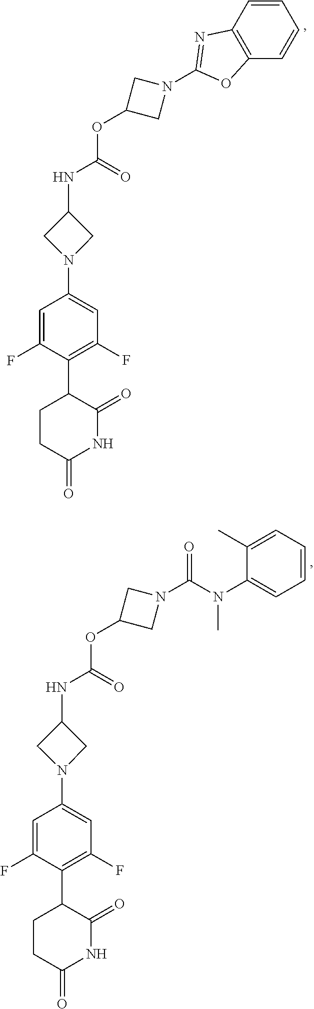 [SMILES: O=C1CCC(c2c(F)cc(N3CC(NC(=O)OC4CN(c5nc6ccccc6o5)C4)C3)cc2F)C(=O)N1 ; Cc1ccccc1N(C)C(=O)N1CC(OC(=O)NC2CN(c3cc(F)c(C4CCC(=O)NC4=O)c(F)c3)C2)C1] 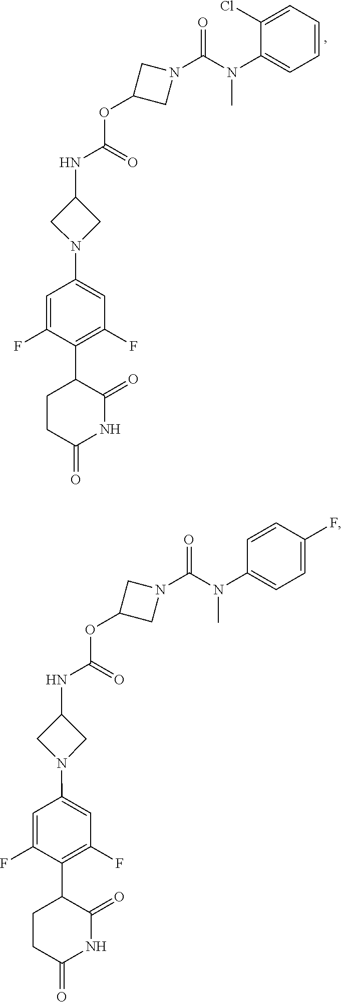 [SMILES: CN(C(=O)N1CC(OC(=O)NC2CN(c3cc(F)c(C4CCC(=O)NC4=O)c(F)c3)C2)C1)c1ccccc1Cl ; CN(C(=O)N1CC(OC(=O)NC2CN(c3cc(F)c(C4CCC(=O)NC4=O)c(F)c3)C2)C1)c1ccc(F)cc1] 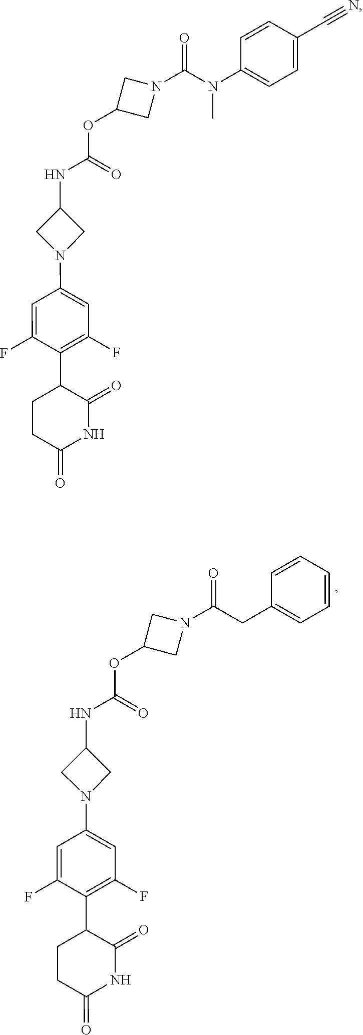 [SMILES: CN(C(=O)N1CC(OC(=O)NC2CN(c3cc(F)c(C4CCC(=O)NC4=O)c(F)c3)C2)C1)c1ccc(C#N)cc1 ; O=C1CCC(c2c(F)cc(N3CC(NC(=O)OC4CN(C(=O)Cc5ccccc5)C4)C3)cc2F)C(=O)N1] 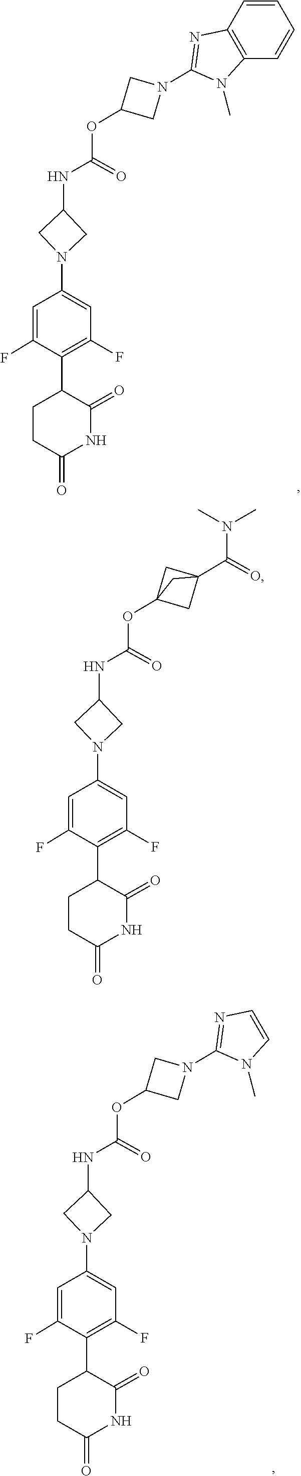 [SMILES: Cn1c(N2CC(OC(=O)NC3CN(c4cc(F)c(C5CCC(=O)NC5=O)c(F)c4)C3)C2)nc2ccccc21 ; CN(C)C(=O)C12CC(OC(=O)NC3CN(c4cc(F)c(C5CCC(=O)NC5=O)c(F)c4)C3)(C1)C2 ; Cn1ccnc1N1CC(OC(=O)NC2CN(c3cc(F)c(C4CCC(=O)NC4=O)c(F)c3)C2)C1] 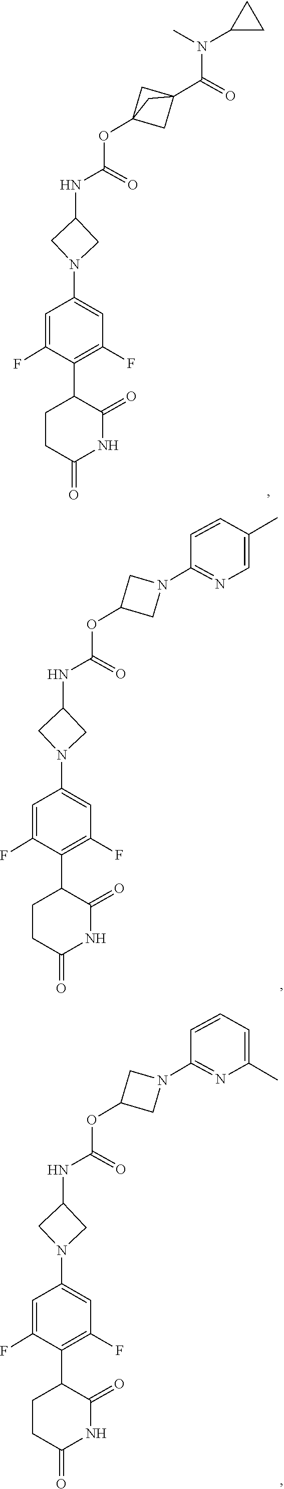 [SMILES: CN(C(=O)C12CC(OC(=O)NC3CN(c4cc(F)c(C5CCC(=O)NC5=O)c(F)c4)C3)(C1)C2)C1CC1 ; Cc1ccc(N2CC(OC(=O)NC3CN(c4cc(F)c(C5CCC(=O)NC5=O)c(F)c4)C3)C2)nc1 ; Cc1cccc(N2CC(OC(=O)NC3CN(c4cc(F)c(C5CCC(=O)NC5=O)c(F)c4)C3)C2)n1] 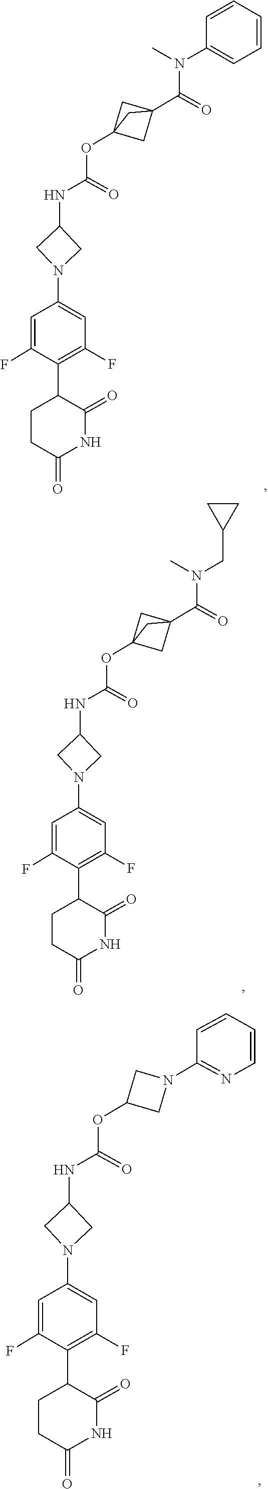 [SMILES: CN(C(=O)C12CC(OC(=O)NC3CN(c4cc(F)c(C5CCC(=O)NC5=O)c(F)c4)C3)(C1)C2)c1ccccc1 ; CN(CC1CC1)C(=O)C12CC(OC(=O)NC3CN(c4cc(F)c(C5CCC(=O)NC5=O)c(F)c4)C3)(C1)C2 ; O=C1CCC(c2c(F)cc(N3CC(NC(=O)OC4CN(c5ccccn5)C4)C3)cc2F)C(=O)N1] 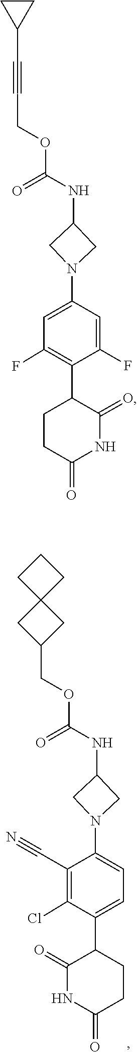 [SMILES: O=C1CCC(c2c(F)cc(N3CC(NC(=O)OCC#CC4CC4)C3)cc2F)C(=O)N1 ; N#Cc1c(N2CC(NC(=O)OCC3CC4(CCC4)C3)C2)ccc(C2CCC(=O)NC2=O)c1Cl] 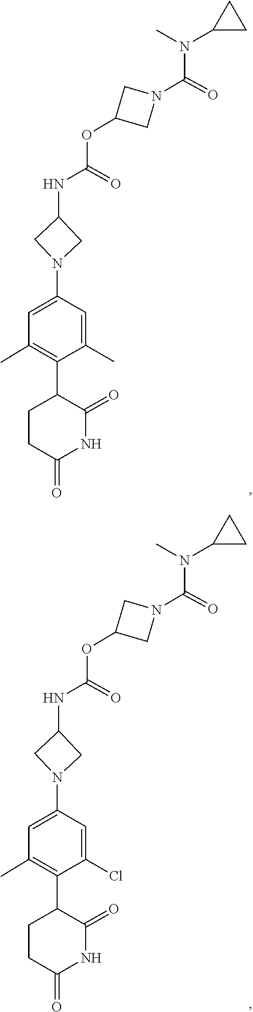 [SMILES: Cc1cc(N2CC(NC(=O)OC3CN(C(=O)N(C)C4CC4)C3)C2)cc(C)c1C1CCC(=O)NC1=O ; Cc1cc(N2CC(NC(=O)OC3CN(C(=O)N(C)C4CC4)C3)C2)cc(Cl)c1C1CCC(=O)NC1=O] 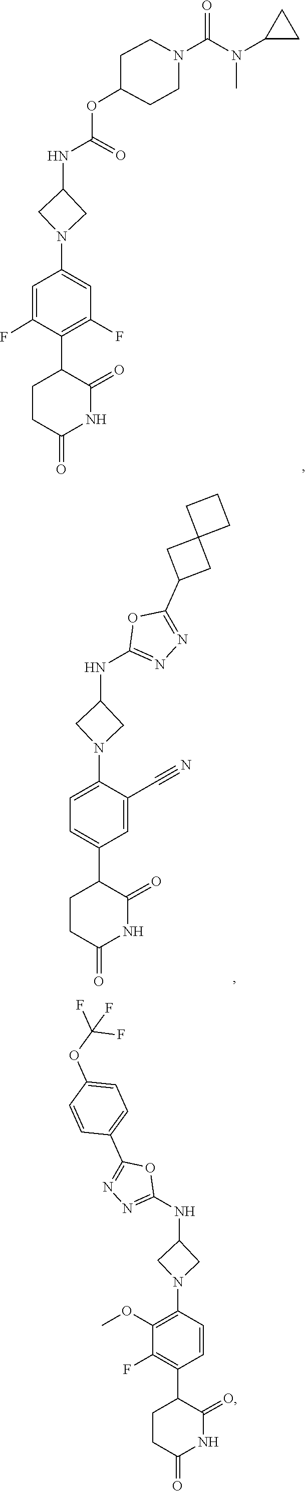 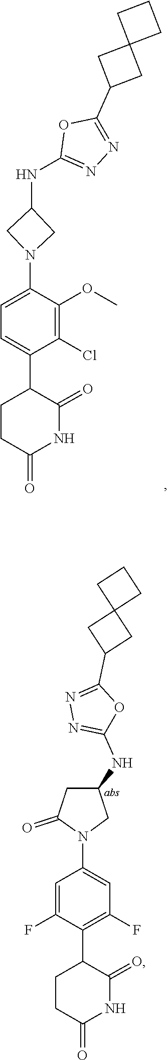 [SMILES: COc1c(N2CC(Nc3nnc(C4CC5(CCC5)C4)o3)C2)ccc(C2CCC(=O)NC2=O)c1Cl ; O=C1CCC(c2c(F)cc(N3C[C@H](Nc4nnc(C5CC6(CCC6)C5)o4)CC3=O)cc2F)C(=O)N1] 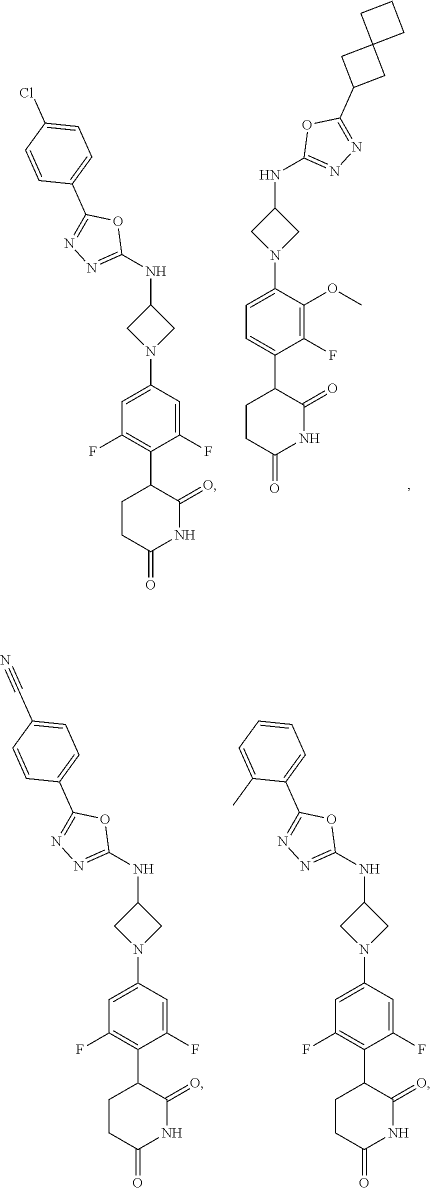 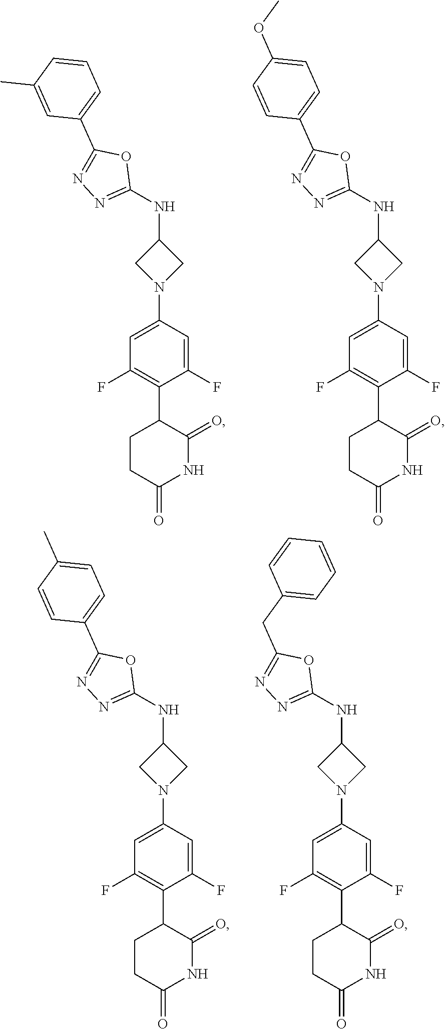 [SMILES: Cc1cccc(-c2nnc(NC3CN(c4cc(F)c(C5CCC(=O)NC5=O)c(F)c4)C3)o2)c1 ; COc1ccc(-c2nnc(NC3CN(c4cc(F)c(C5CCC(=O)NC5=O)c(F)c4)C3)o2)cc1 ; Cc1ccc(-c2nnc(NC3CN(c4cc(F)c(C5CCC(=O)NC5=O)c(F)c4)C3)o2)cc1 ; O=C1CCC(c2c(F)cc(N3CC(Nc4nnc(Cc5ccccc5)o4)C3)cc2F)C(=O)N1] 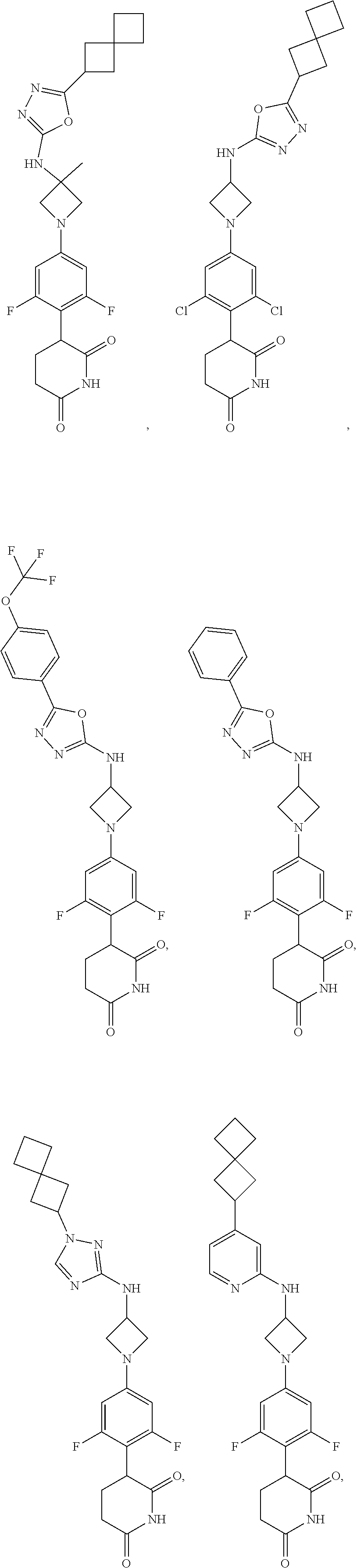 [SMILES: CC1(Nc2nnc(C3CC4(CCC4)C3)o2)CN(c2cc(F)c(C3CCC(=O)NC3=O)c(F)c2)C1 ; O=C1CCC(c2c(Cl)cc(N3CC(Nc4nnc(C5CC6(CCC6)C5)o4)C3)cc2Cl)C(=O)N1 ; O=C1CCC(c2c(F)cc(N3CC(Nc4nnc(-c5ccc(OC(F)(F)F)cc5)o4)C3)cc2F)C(=O)N1 ; O=C1CCC(c2c(F)cc(N3CC(Nc4nnc(-c5ccccc5)o4)C3)cc2F)C(=O)N1 ; O=C1CCC(c2c(F)cc(N3CC(Nc4ncn(C5CC6(CCC6)C5)n4)C3)cc2F)C(=O)N1 ; O=C1CCC(c2c(F)cc(N3CC(Nc4cc(C5CC6(CCC6)C5)ccn4)C3)cc2F)C(=O)N1] 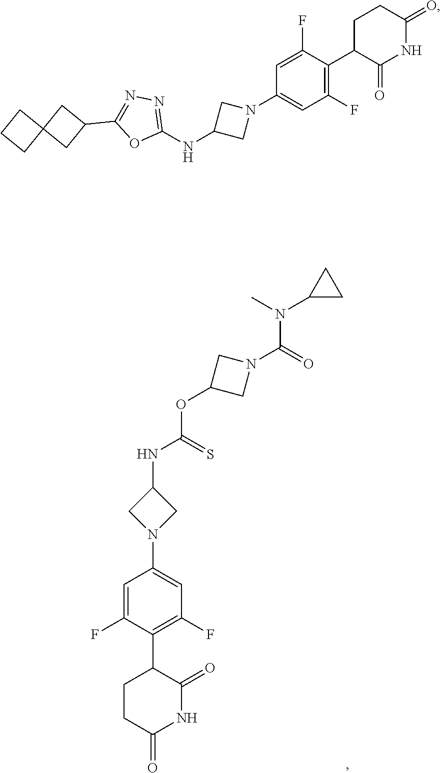 [SMILES: O=C1CCC(c2c(F)cc(N3CC(Nc4nnc(C5CC6(CCC6)C5)o4)C3)cc2F)C(=O)N1 ; CN(C(=O)N1CC(OC(=S)NC2CN(c3cc(F)c(C4CCC(=O)NC4=O)c(F)c3)C2)C1)C1CC1] 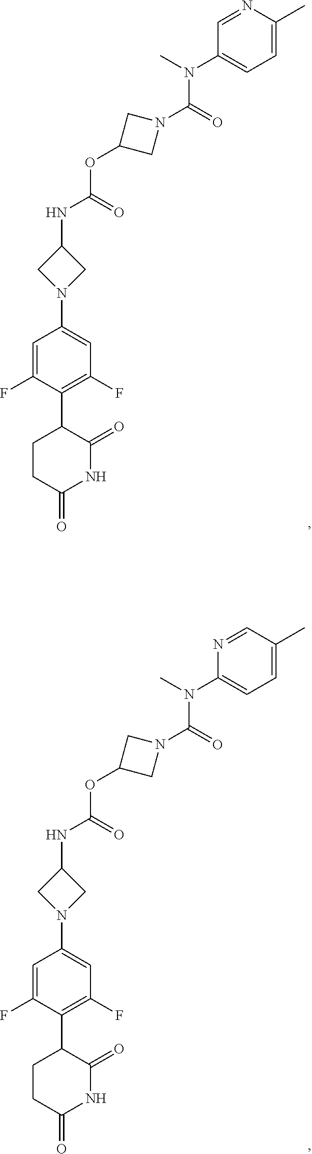 [SMILES: Cc1ccc(N(C)C(=O)N2CC(OC(=O)NC3CN(c4cc(F)c(C5CCC(=O)NC5=O)c(F)c4)C3)C2)cn1 ; Cc1ccc(N(C)C(=O)N2CC(OC(=O)NC3CN(c4cc(F)c(C5CCC(=O)NC5=O)c(F)c4)C3)C2)nc1] 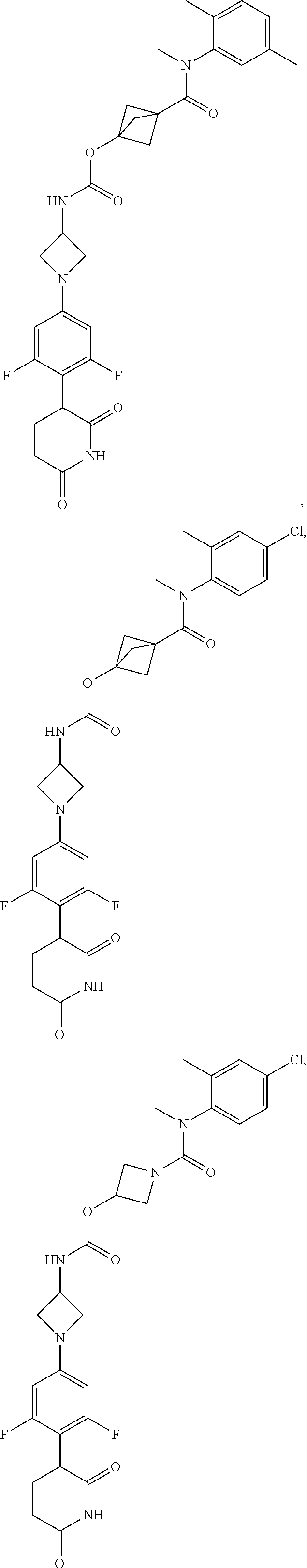 [SMILES: Cc1ccc(C)c(N(C)C(=O)C23CC(OC(=O)NC4CN(c5cc(F)c(C6CCC(=O)NC6=O)c(F)c5)C4)(C2)C3)c1 ; Cc1cc(Cl)ccc1N(C)C(=O)C12CC(OC(=O)NC3CN(c4cc(F)c(C5CCC(=O)NC5=O)c(F)c4)C3)(C1)C2 ; Cc1cc(Cl)ccc1N(C)C(=O)N1CC(OC(=O)NC2CN(c3cc(F)c(C4CCC(=O)NC4=O)c(F)c3)C2)C1] 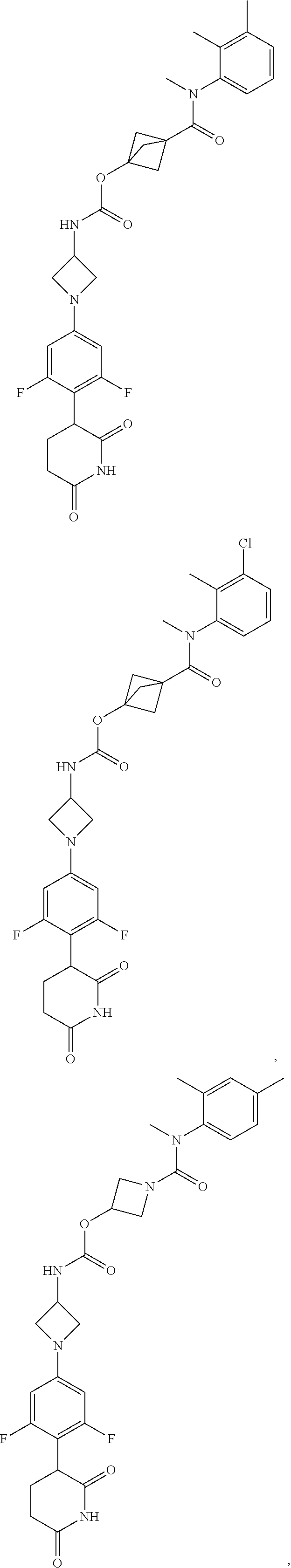 [SMILES: Cc1cccc(N(C)C(=O)C23CC(OC(=O)NC4CN(c5cc(F)c(C6CCC(=O)NC6=O)c(F)c5)C4)(C2)C3)c1C ; Cc1c(Cl)cccc1N(C)C(=O)C12CC(OC(=O)NC3CN(c4cc(F)c(C5CCC(=O)NC5=O)c(F)c4)C3)(C1)C2 ; Cc1ccc(N(C)C(=O)N2CC(OC(=O)NC3CN(c4cc(F)c(C5CCC(=O)NC5=O)c(F)c4)C3)C2)c(C)c1] 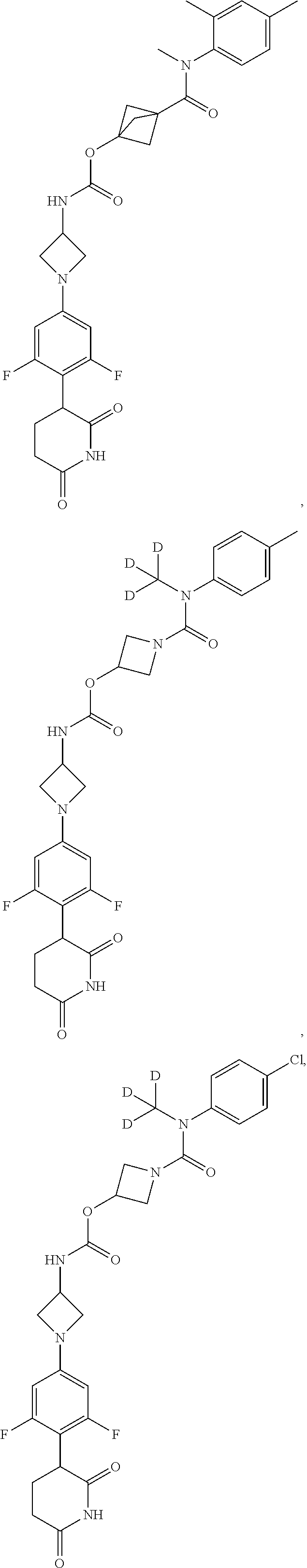 [SMILES: Cc1ccc(N(C)C(=O)C23CC(OC(=O)NC4CN(c5cc(F)c(C6CCC(=O)NC6=O)c(F)c5)C4)(C2)C3)c(C)c1 ; [2H]C([2H])([2H])N(C(=O)N1CC(OC(=O)NC2CN(c3cc(F)c(C4CCC(=O)NC4=O)c(F)c3)C2)C1)c1ccc(C)cc1 ; [2H]C([2H])([2H])N(C(=O)N1CC(OC(=O)NC2CN(c3cc(F)c(C4CCC(=O)NC4=O)c(F)c3)C2)C1)c1ccc(Cl)cc1] 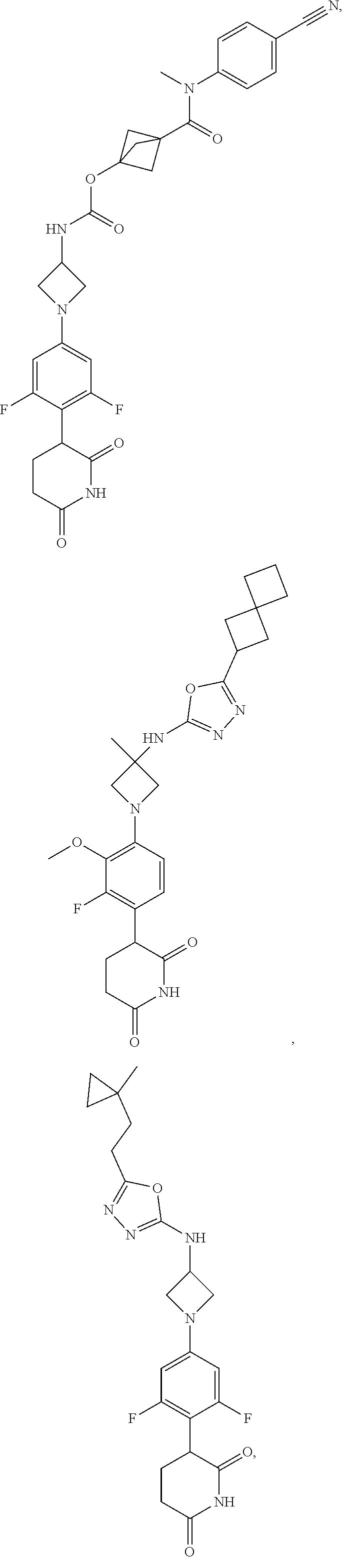 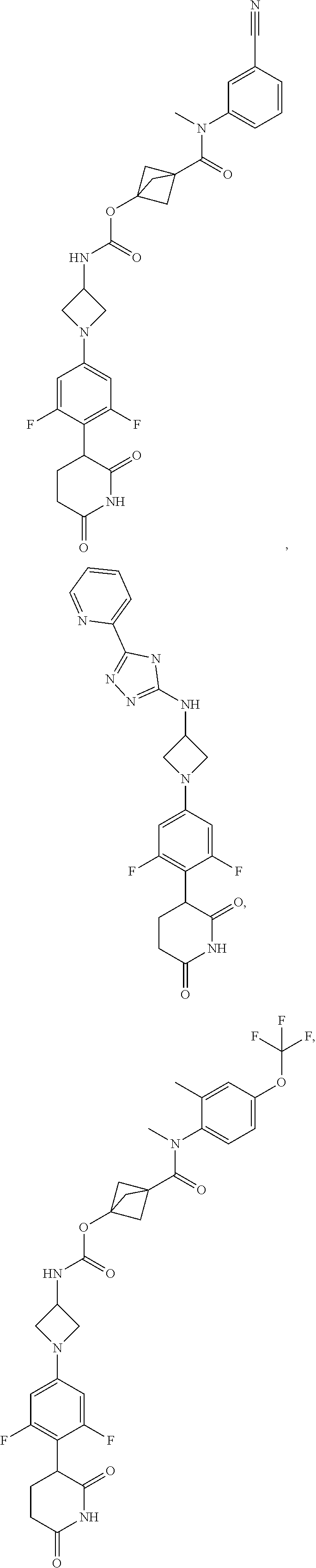 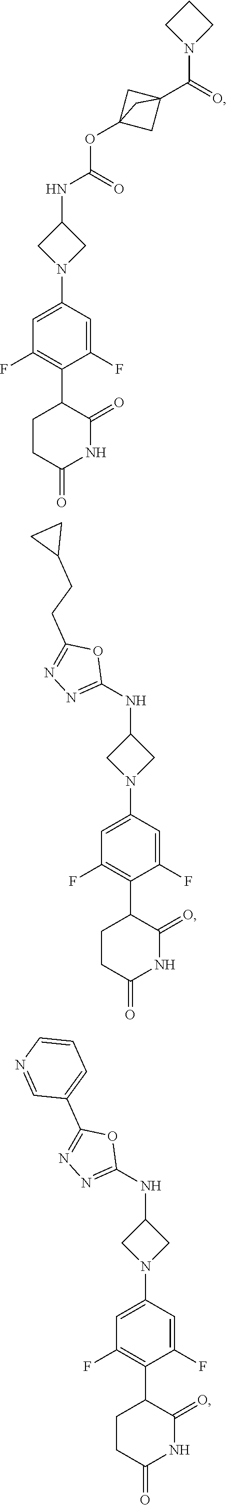 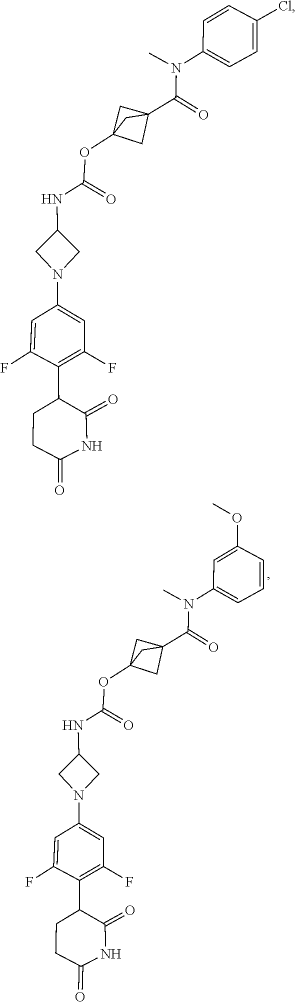 [SMILES: CN(C(=O)C12CC(OC(=O)NC3CN(c4cc(F)c(C5CCC(=O)NC5=O)c(F)c4)C3)(C1)C2)c1ccc(Cl)cc1 ; COc1cccc(N(C)C(=O)C23CC(OC(=O)NC4CN(c5cc(F)c(C6CCC(=O)NC6=O)c(F)c5)C4)(C2)C3)c1] 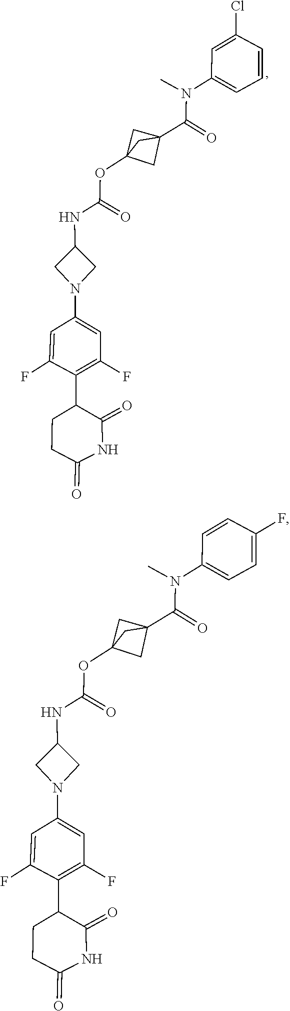 [SMILES: CN(C(=O)C12CC(OC(=O)NC3CN(c4cc(F)c(C5CCC(=O)NC5=O)c(F)c4)C3)(C1)C2)c1cccc(Cl)c1 ; CN(C(=O)C12CC(OC(=O)NC3CN(c4cc(F)c(C5CCC(=O)NC5=O)c(F)c4)C3)(C1)C2)c1ccc(F)cc1] 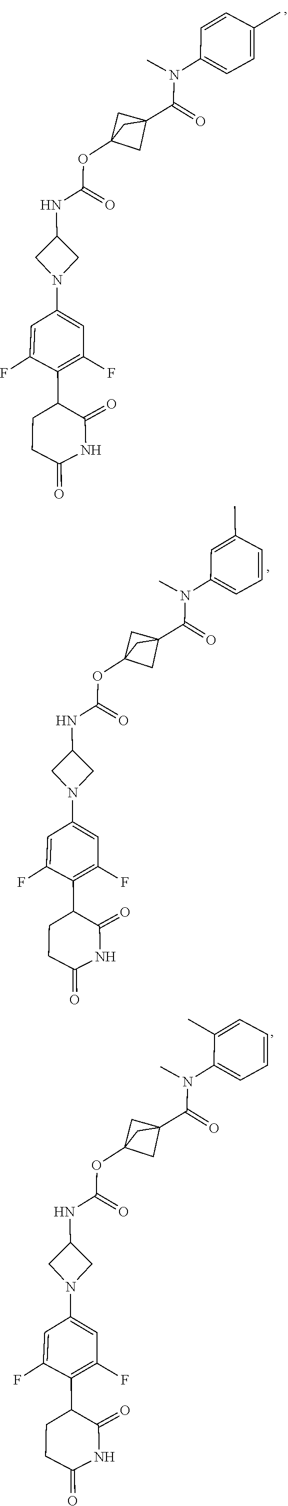 [SMILES: Cc1ccc(N(C)C(=O)C23CC(OC(=O)NC4CN(c5cc(F)c(C6CCC(=O)NC6=O)c(F)c5)C4)(C2)C3)cc1 ; Cc1cccc(N(C)C(=O)C23CC(OC(=O)NC4CN(c5cc(F)c(C6CCC(=O)NC6=O)c(F)c5)C4)(C2)C3)c1 ; Cc1ccccc1N(C)C(=O)C12CC(OC(=O)NC3CN(c4cc(F)c(C5CCC(=O)NC5=O)c(F)c4)C3)(C1)C2] 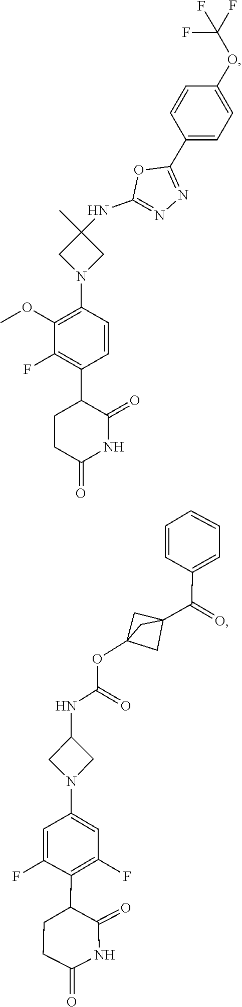 [SMILES: COc1c(N2CC(C)(Nc3nnc(-c4ccc(OC(F)(F)F)cc4)o3)C2)ccc(C2CCC(=O)NC2=O)c1F ; O=C1CCC(c2c(F)cc(N3CC(NC(=O)OC45CC(C(=O)c6ccccc6)(C4)C5)C3)cc2F)C(=O)N1] 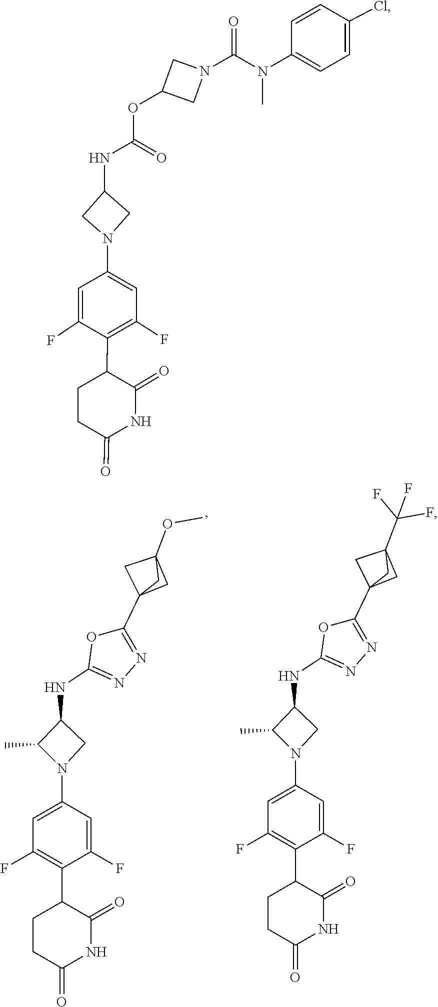 [SMILES: CN(C(=O)N1CC(OC(=O)NC2CN(c3cc(F)c(C4CCC(=O)NC4=O)c(F)c3)C2)C1)c1ccc(Cl)cc1 ; COC12CC(c3nnc(N[C@H]4CN(c5cc(F)c(C6CCC(=O)NC6=O)c(F)c5)[C@@H]4C)o3)(C1)C2 ; C[C@@H]1[C@@H](Nc2nnc(C34CC(C(F)(F)F)(C3)C4)o2)CN1c1cc(F)c(C2CCC(=O)NC2=O)c(F)c1] 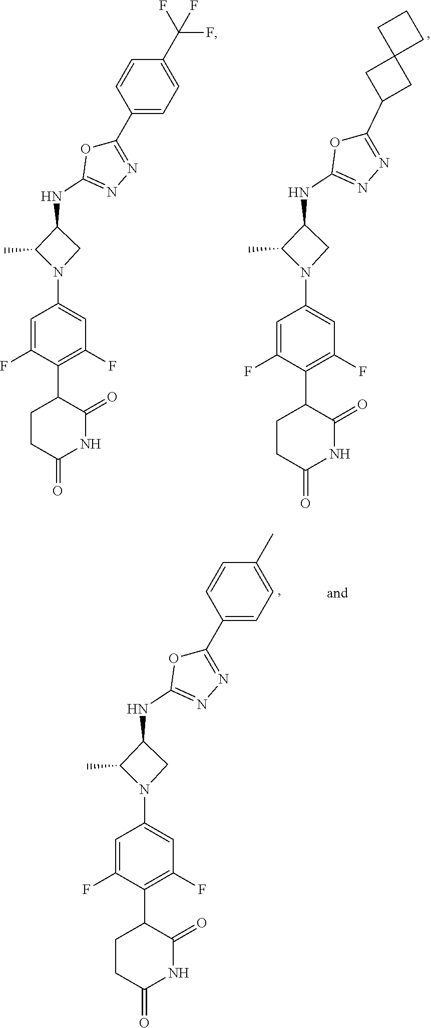 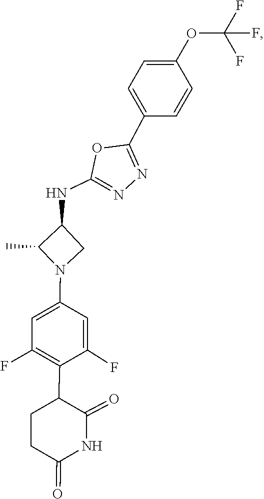 [SMILES: C[C@@H]1[C@@H](Nc2nnc(-c3ccc(OC(F)(F)F)cc3)o2)CN1c1cc(F)c(C2CCC(=O)NC2=O)c(F)c1]
or a pharmaceutically acceptable salt thereof.

32. A pharmaceutical composition comprising the compound of any one of claims 1-31, or pharmaceutically acceptable salt thereof, and a pharmaceutically acceptable excipient.

33. A method of degrading CDK2 in a subject suffering from cancer, comprising administering to the subject an effective amount of the compound of any one of claims 1-31, or pharmaceutically acceptable salt thereof, or the pharmaceutical composition of claim 32.

34. A method of treating cancer in a subject in need thereof, comprising administering to the subject a therapeutically effective amount of the compound of any one of claims 1-31, or pharmaceutically acceptable salt thereof, or the pharmaceutical composition of claim 32.

35. The method of claim 33 or 34, wherein the cancer is breast cancer.

36. The method of claim 35, wherein the breast cancer is triple negative breast cancer or estrogen receptor positive breast cancer.

37. A method of treating a solid tumor in a subject in need thereof, comprising administering to the subject a therapeutically effective amount of the compound of any one of claims 1-31, or pharmaceutically acceptable salt thereof, or the pharmaceutical composition of claim 32.

38. A method of treating a liquid tumor in a subject in need thereof, comprising administering to the subject a therapeutically effective amount of the compound of any one of claims 1-31, or pharmaceutically acceptable salt thereof, or the pharmaceutical composition of claim 32.

39. The method of any one of claims 33-38, further comprising administering to the subject an additional therapeutic agent.
